(19) United States
(12) Patent Application Publication
(10) Pub. No.: US 2024/0366770 A1
(43) Pub. Date: Nov. 7, 2024

(54) DEGRADATION OF BRUTON'S TYROSINE KINASE (BTK) BY CONJUGATION OF BTK INHIBITORS WITH E3 LIGASE LIGAND AND METHODS OF USE

(71) Applicant: BeiGene Switzerland GmbH, Basel (CH)

(72) Inventors: Changxin HUO, Beijing (CN); Hexiang WANG, Beijing (CN); Zhemin CHEN, Beijing (CN); Zhiwei WANG, Beijing (CN); Huaqing LIU, Beijing (CN)

(73) Assignee: BeiGene Switzerland GmbH, Basel (CH)

(21) Appl. No.: 18/758,275

(22) Filed: Jun. 28, 2024

Related U.S. Application Data
(63) Continuation of application No. PCT/CN2022/143835, filed on Dec. 30, 2022.

(30) Foreign Application Priority Data
Dec. 30, 2021 (WO) PCT/CN2021/142803

(51) Int. Cl. A61K 47/55; A61K 47/54

(52) U.S. Cl. CPC A61K 47/55; A61K 47/545

(57) ABSTRACT

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis-targeting chimera (PROTAC) is a novel strategy for selective knockdown of target proteins by small molecules (Sakamoto K M et al., Proc Natl Acad Sci 2001, 98:8554-9.; Sakamoto K. M. et al., Methods Enzymol. 2005; 399:833-847.). PROTAC utilizes the ubiquitin-protease system to target a specific protein and induce its degradation in the cell (Zhou P. et al., Mol Cell. 2000; 6(3):751-756; Neklesa T. K. et al., Pharmacol Ther. 2017; 174:138-144; Lu M. et al., Eur J Med Chem. 2018; 146:251-259). The normal physiological function of the ubiquitin-protease system is responsible for clearing denatured, mutated, or harmful proteins in cells. The normal physiological function of the ubiquitin-protease system is responsible for clearing denatured, mutated, or harmful proteins in cells. The ubiquitin-proteasome system (UPS), also known as the ubiquitin-proteasome pathway (UPP), is a common posttranslational regulation mechanism that is responsible for protein degradation in normal and pathological states (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Ubiquitin, which is highly conserved in eukaryotic cells, is a modifier molecule, composed of 76 amino acids, that covalently binds to and labels target substrates via a cascade of enzymatic reactions involving E1, E2, and E3 enzymes. Subsequently, the modified substrate is recognized by the 26S proteasome complex for ubiquitination-mediated degradation. So far, two E1 enzymes have been discovered, which are termed UBA1 and UBA6. On the other hand, there are about 40 E2 enzymes and more than 600 E3 enzymes that offer the functional diversity to govern the activity of many downstream protein substrates. However, only a limited number of E3 ubiquitin ligases have been successfully hijacked for use by small-molecule PROTAC technology: the Von Hippel-Lindau disease tumor suppressor protein (VHL), the Mouse Double Minute 2 homologue (MDM2), the Cellular Inhibitor of Apoptosis (cIAP), and cereblon (Philipp O. et al., Chem. Biol. 2017, 12, 2570-2578).

Bifunctional compounds composed of a target protein-binding moiety and an E3 ubiquitin ligase-binding moiety have been shown to induce proteasome-mediated degradation of selected proteins. These drug-like molecules offer the possibility of temporal control over protein expression and could be useful as biochemical reagents for the treatment of diseases. In recent years, this newly developed method has been widely used in antitumor studies (Lu J. et al., Chem Biol. 2015; 22(6):755-763; Ottis P. et al., Chem Biol. 2017; 12(4):892-898.; Crews C. M. et al., J Med Chem. 2018; 61(2):403-404; Neklesa T. K. et al., Pharmacol Ther. 2017, 174:138-144.; Cermakova K. et al., Molecules, 2018.23(8).; An S. et al., EBioMedicine, 2018.; Lebraud H. et al., Essays Biochem. 2017; 61(5): 517-527.; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); Shenxin Z. et al., European Journal of Medicinal Chemistry, 2020, doi.org/10.1016/j.ejmech.2020.112981) and has been disclosed or discussed in patent publications, e.g., US20160045607, US20170008904, US20180050021, US20180072711, WO2002020740, WO2014108452, WO2016146985, WO2016149668, WO2016149989, WO2016197032, WO2016197114, WO2017011590, WO2017030814, WO2017079267, WO2017182418, WO2017197036, WO2017197046, WO2017197051, WO2017197056, WO2017201449, WO2017211924, WO2018033556, WO2018071606, US 20200297725, US20200239430, WO 2020142228, WO2020163823, WO2020198711, WO2020201080, et al.

Bruton's tyrosine kinase (Btk) belongs to the Tec tyrosine kinase family (Vetrie et al., Nature 361: 226-233, 1993; Bradshaw, Cell Signal. 22: 1175-84, 2010). Btk is primarily expressed in most hematopoietic cells such as B cells, mast cells and macrophages (Smith et al., J. Immunol. 152: 557-565, 1994) and is localized in bone marrow, spleen and lymph node tissue. Btk plays important roles in B-cell receptor (BCR) and FcR signaling pathways, which involve in B-cell development, differentiation (Khan, Immunol. Res. 23: 147, 2001). Btk is activated by upstream Src-family kinases. Once activated, Btk, in turn, phosphorylates PLC gamma, leading to effects on B-cell function and survival (Humphries et al., J. Biol. Chem. 279: 37651, 2004). These signaling pathways must be precisely regulated. Mutations in the gene encoding Btk cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that Btk deficient mice are resistant to developing collagen-induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, Btk inhibitors can be used to treat autoimmune and/or inflammatory diseases.

Inhibition of BTK has been shown to affect cancer development (B cell malignancies) and cell viability, and improve autoimmune diseases (e.g., rheumatoid arthritis and lupus). Inhibition of BTK has also been reported via alternative strategies, such as through degradation of BTK (Alexandru D. et al., Biochemistry 2018, 57, 26, 3564-3575; Adelajda Z. et al., PNAS 2018 115 (31); Dennis D., et al., Blood, 2019, 133:952-961; Yonghui S. et al., Cell Research, 2018, 28, 779-781; Yonghui S. et al., Leukemia, 2019, Degradation of Bruton's tyrosine kinase mutants by PROTACs for potential treatment of ibrutinib-resistant non-Hodgkin lymphomas) and has been disclosed or discussed in patent publications, e.g. US20190276459, WO2019186343, WO2019186358, WO2019148150, WO2019177902, WO2019127008, and WO2020239103.

There is a need of new BTK inhibitors or degraders which are more potent than known inhibitors of BTK and inhibit BTK via alternative strategies, such as through degradation of BTK. The present application addresses the need.

### SUMMARY OF THE INVENTION

The following aspects or embodiments are parts of the instant invention. However, the instant invention is not limited thereto.

Aspect 1. A compound of Formula (I):

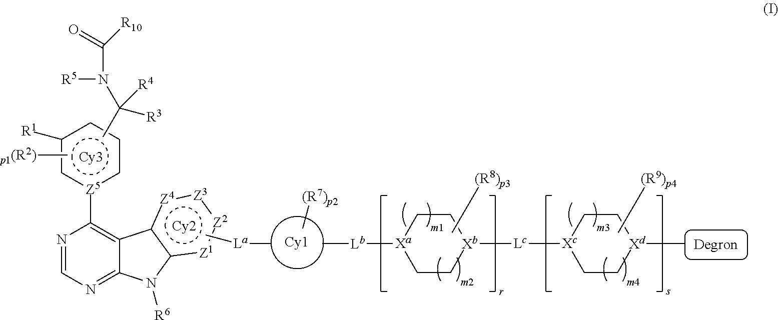

or a pharmaceutically acceptable salt thereof, or a stereoisomer thereof, wherein:
Cy1 is a 4-, 5-, 6-, 7-, 8-, 9-, 10-, 11- or 12-membered ring comprising 0-3 heteroatoms selected from nitrogen, oxygen and sulfur;
Cy2 is a ring fused to the structure

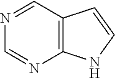

in Formula (I), said ring is an aromatic ring or a non-aromatic ring (preferably aromatic ring);
Cy3 is a saturated ring or unsaturated ring (preferably aromatic ring);
L^{a}, L^{b} and L^{c} are each independently a single bond, —O—, —S—, —C(O)—, —(CR^{c}R^{d})ₙ— or —NR^{c}—;
the [symbol] moiety is an E3 Ubiquitin ligase moiety selected from

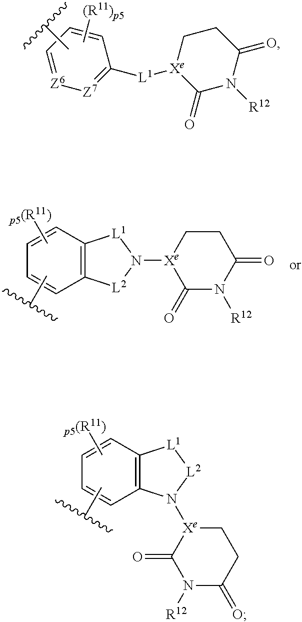

L¹ and L² are each independently a single bond, —O—, —S—, —C(O)—, —(CR^{c}R^{d})ₙ— or —NR^{c}—;
Z¹ is N or CR^{za} if Z¹ is a part of a double bond in the ring (also referred to as ═N— or ═CR^{za}—); or Z¹ is NR^{za} or C(R^{za})₂ if Z¹ is not a part of a double bond in the ring (also referred to as —NR^{za}— or —C(R^{za})₂—);
Z² is N or CR^{za} if Z² is a part of a double bond in the ring (also referred to as ═N— or ═CR^{za}—); or Z² is NR^{za} or C(R^{za})₂ if Z² is not a part of a double bond in the ring (also referred to as —NR^{za}— or —C(R^{za})₂—);
Z³ is N or CR^{za} if Z³ is a part of a double bond in the ring (also referred to as ═N— or ═C(R^{za})—); or Z³ is NR^{za} or C(R^{za})₂ if Z³ is not a part of a double bond in the ring (also referred to as —NR^{za}— or —C(R^{za})₂—);
Z⁴ is N or CR^{za} if Z⁴ is a part of a double bond in the ring (also referred to as ═N— or ═CR^{za}—); or Z⁴ is NR^{za} or C(R^{za})₂ if Z⁴ is not a part of a double bond in the ring (also referred to as —NR^{za}— or —C(R^{za})₂—); provided that not all Z¹, Z², Z³ and Z⁴ are ═N—;
Z⁵ is C if Z⁵ is a part of a double bond in the ring; or Z⁵ is N or CR^{za} if Z⁵ is not a part of a double bond in the ring;
Z⁶ and Z⁷ are each independently CR^{za} or N;
X^{a}, X^{b}, X^{c}, X^{d} and X^{e} are each independently CR¹³ or N;
each of occurrence, R¹, R², R³, R⁴, R⁷, R⁸, R⁹, R¹¹, R¹³ and R^{za} are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
or R¹ and R³, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R⁵, R⁶ and R¹² are each independently hydrogen, —C₁₋₈alkyl, —C₂₋₈alkenyl, —C₂₋₈alkynyl, cycloalkyl, heterocyclyl, aryl or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R¹⁰ is 5- or 6-membered aromatic ring comprising 0-3 heteroatoms selected from nitrogen, oxygen and sulfur; said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with —C₁₋₈alkyl, halogen, hydroxy, -haloC₁₋₈alkyl, —C₁₋₈alkyoxy, cycloalkyl, heterocyclyl, aryl, or heteroaryl;
or R⁵ and R¹⁰, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
m1, m2, m3, m4, p1, p2, p3, p4 and p5 are each independently 0, 1, 2, 3 or 4;
n is 0, 1, 2, 3, 4, 5 or 6;
r is 0 or 1;
s is 0 or 1;
R^{a} and R^{b} are each independently hydrogen, —C₁₋₈alkyl, —C₂₋₈alkenyl, —C₂₋₈alkynyl, cycloalkyl, heterocyclyl, aryl, or heteroaryl; or
R^{a} and R^{b} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂;
R^{c} and R^{d} are each independently hydrogen, —C₁₋₈alkyl, —C₂₋₈alkenyl, —C₂₋₈alkynyl, cycloalkyl, heterocyclyl, aryl, or heteroaryl; or
R^{c} and R^{d} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

In some embodiments, at least one of Z² and Z³ is ═CH— if a part of a double bond in the ring or —CH₂— if not a part of a double bond in the ring, either the ═CH— or —CH₂— moiety provides the position to be linked with the

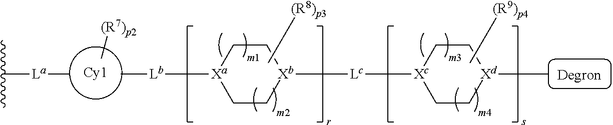

moiety.

Aspect 2. The compound according to Aspect 1, wherein the compound is selected from formula (II) and formula (III),

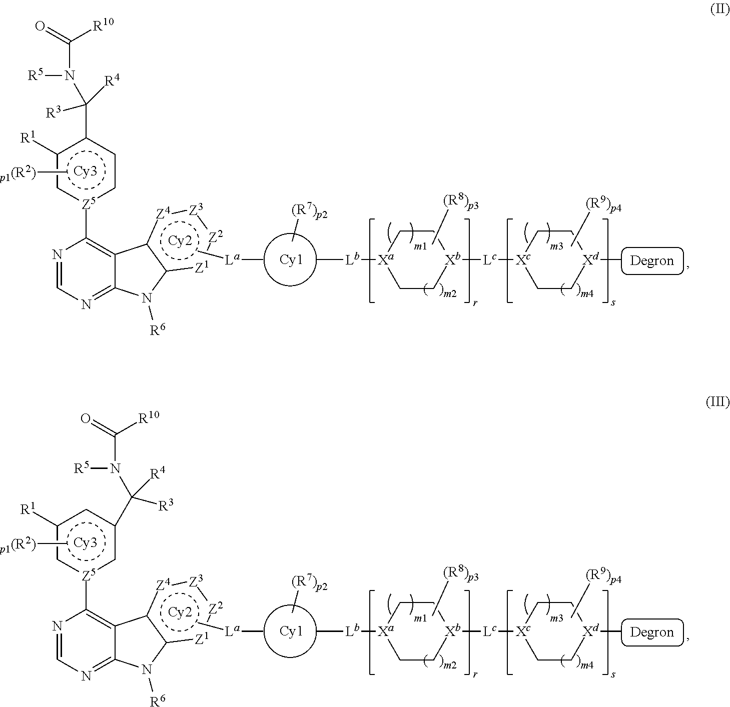

Cy1, Cy2, Cy3, L^{a}, L^{b}, L^{c}, [symbol] , Z¹, Z², Z³, Z⁴, Z⁵, X^{a}, X^{b}, X^{c}, X^{d}, R¹, R², R³, R⁴, R⁵, R⁶, R⁷, R⁸, R⁹, R¹⁰, m1, m2, m3, m4, p1, p2, p3, p4, r and s are defined as claim 1.

Aspect 3. The compound according to Aspect 1, wherein the compound is selected from formula (IV) and formula (V),

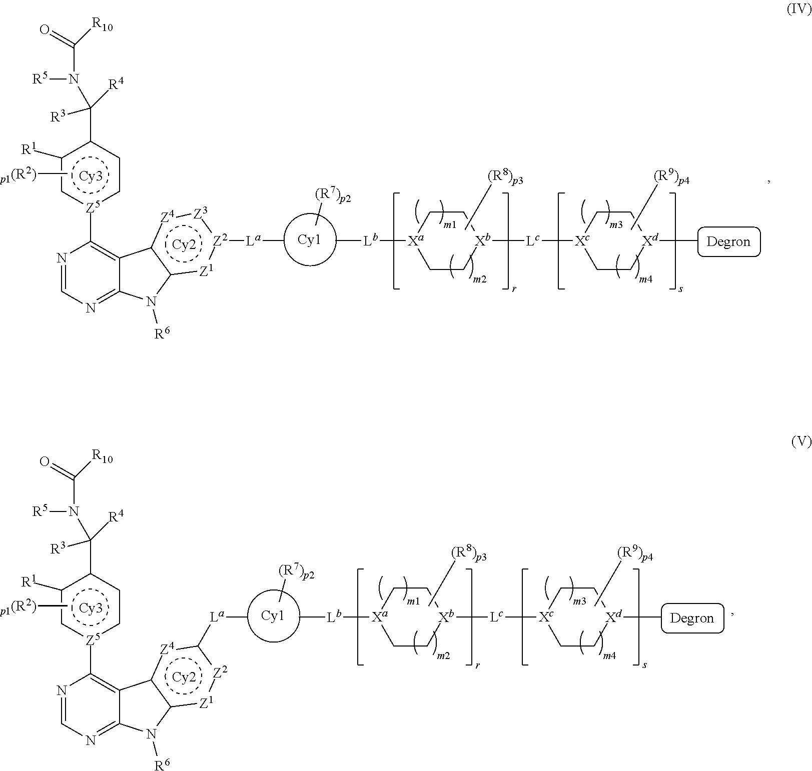

Cy1, Cy2, Cy3, L^{a}, L^{b}, L^{c}, [symbol] , Z¹, Z², Z³, Z⁴, Z⁵, X^{a}, X^{b}, X^{c}, X^{d}, R¹, R², R³, R⁴, R⁵, R⁶, R⁷, R⁸, R⁹, R¹⁰, m1, m2, m3, m4, p1, p2, p3, p4, r and s are defined as claim 1.

Aspect 4. The compound according to Aspect1, wherein Cy1 is

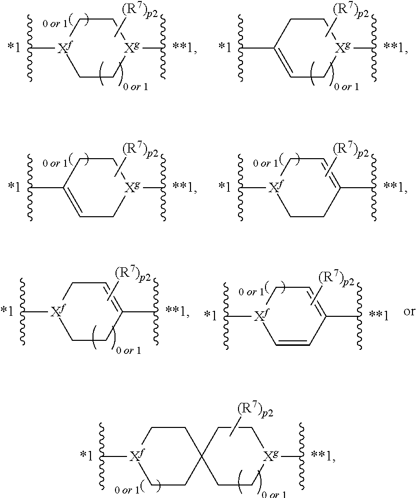

wherein X^{f} and X^{g} are each independently CR¹¹ or N;
1 refers to the position attached to the

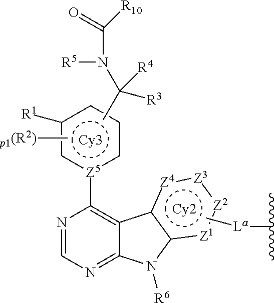

moiety, and **1 refers to the position attached to

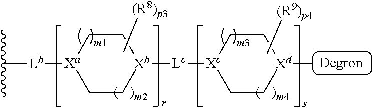

moiety.

Aspect 5. The compound according to Aspect1, wherein Cy1 is

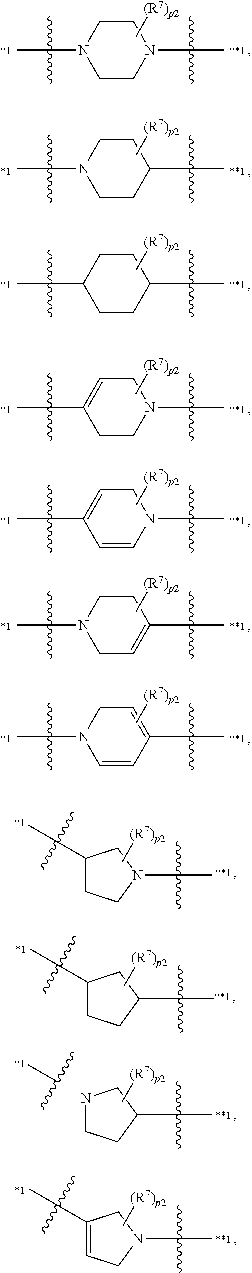

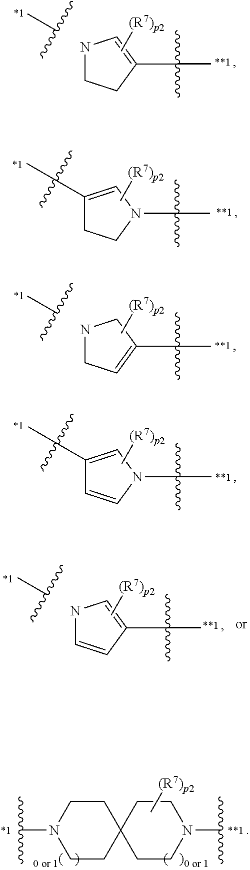

Aspect 6. The compound according to Aspect1, wherein Cy1 is selected from

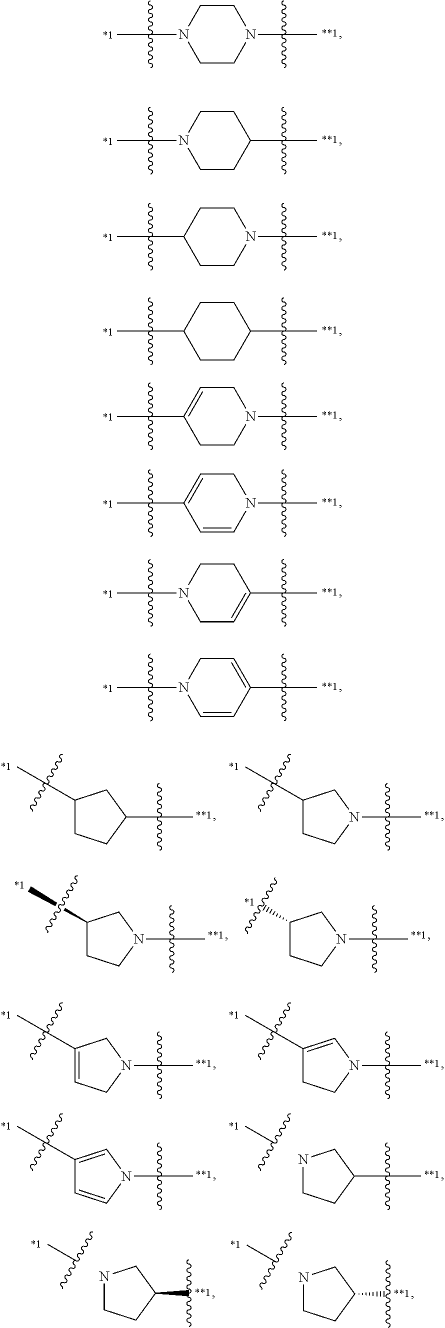

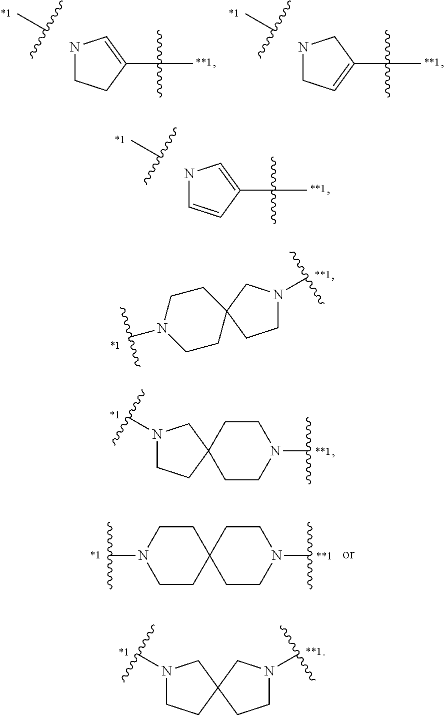

Aspect 7. The compound according to Aspect1, wherein p2 is 0, 1, or 2, and each R⁷ is independently selected hydrogen, —F, —Br, —Cl, —I, —CH₃, —C₂H₅, —C₃H₇, —C₄H₉, —C₅H₁₁, —C₆H₁₃, —C₇H₁₅, —C₈H₁₇, —OCH₃, —OC₂H₅, —OC₃H₇, —OC₄H₉, —OC₅H₁₁, —OC₆H₁₃, —OC₇H₁₅, —OC₈H₁₇, —C_{2- 8}alkenyl, —C₂₋₈ alkynyl, cyclopropyl, cyclobutyl, cyclopentyl, cyclohexyl, cycloheptyl, cyclooctyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —CH₃, —C₂H₅, —C₃H₇, —C₄H₉, —C₅H₁₁, —C₆H₁₃, —C₇H₁₅, —C₈H₁₇, —OCH₃, —OC₂H₅, —OC₃H₇, —OC₄H₉, —OC₅H₁₁, —OC₆H₁₃, —OC₇H₁₅, —OC₈H₁₇, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, is optionally substituted with —F, —Br, —Cl, —I, hydroxy, -haloC₁₋₈alkyl, —C₁₋₈alkyoxy, cycloalkyl, heterocyclyl, aryl, or heteroaryl; R^{a}, R^{b} and R^{c} are defined as claim 1.

Aspect 8. The compound according to Aspect1, wherein p2 is 0, 1, or 2, and each R⁷ is independently selected from hydrogen, —F, —Br, —Cl, —I, —CH₃, —C₂H₅, —C₃H₇, —C₄H₉, —C₅H¹¹, —C₆H₁₃, methoxy, ethoxy, propoxy, butoxy, pentoxy, hexoxy, cyclopropyl, cyclobutyl, cyclopentyl or cyclohexyl.

Aspect 9. The compound according to Aspect1, wherein the [symbol] moiety is selected from

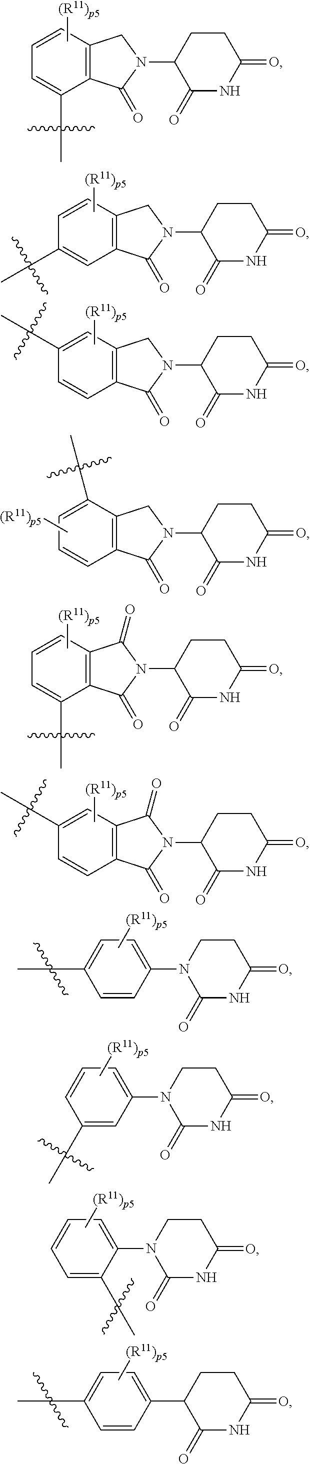

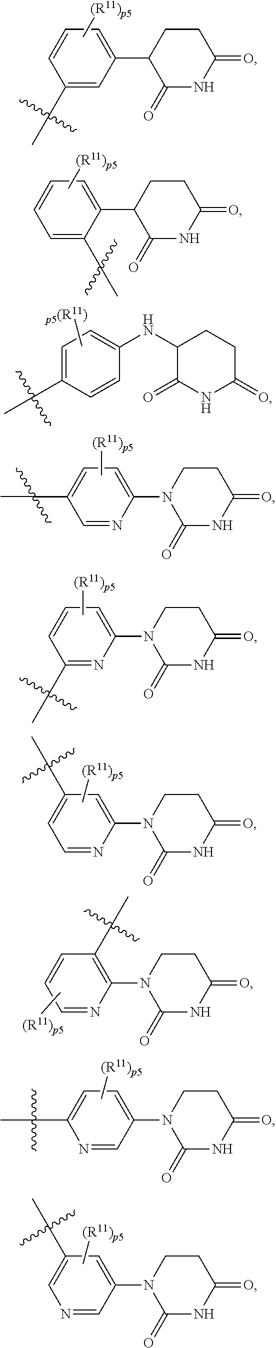

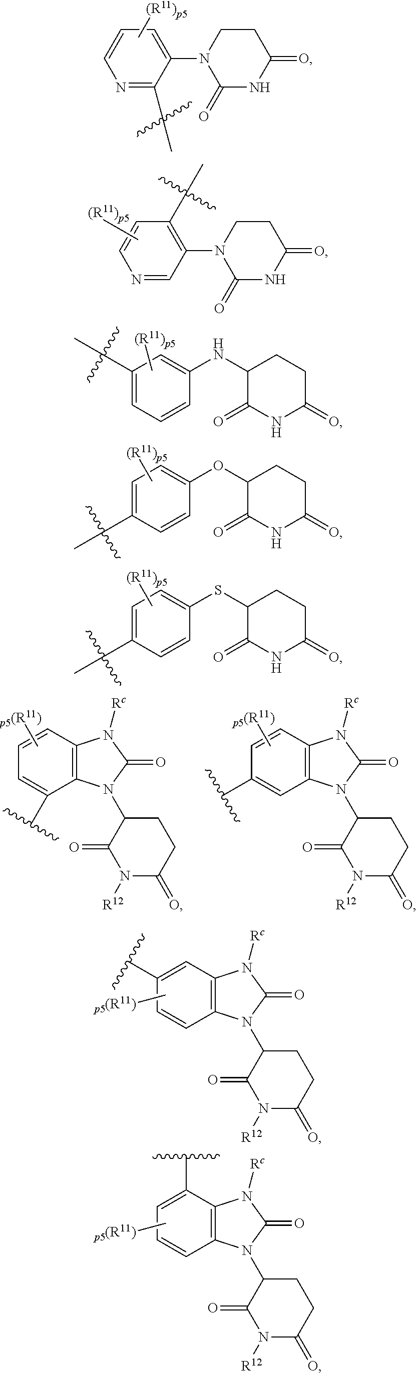

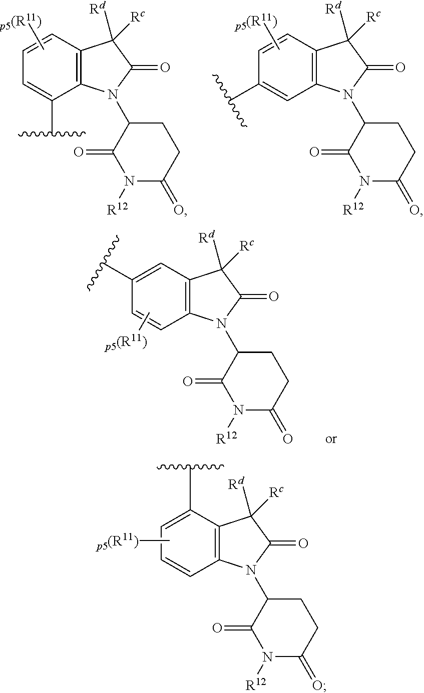

R¹¹ is selected from hydrogen, —F, —Br, —Cl, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl or cyclohexyl;
R^{c} and R^{d} are each independently hydrogen, —CH₃, —C₂H₅, —C₃H₇, —C₄H₉, —C₅H¹¹, —C₆H₁₃, —C₂₋₈alkenyl, —C₂₋₈ alkynyl, cyclopropyl, cyclobutyl, cyclopentyl, cyclohexyl, heterocyclyl, aryl, or heteroaryl; or
R^{c} and R^{d} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂,
and p5 is 0 or 1.

Aspect 10. The compound according to Aspect6, wherein [symbol] the moiety is selected from

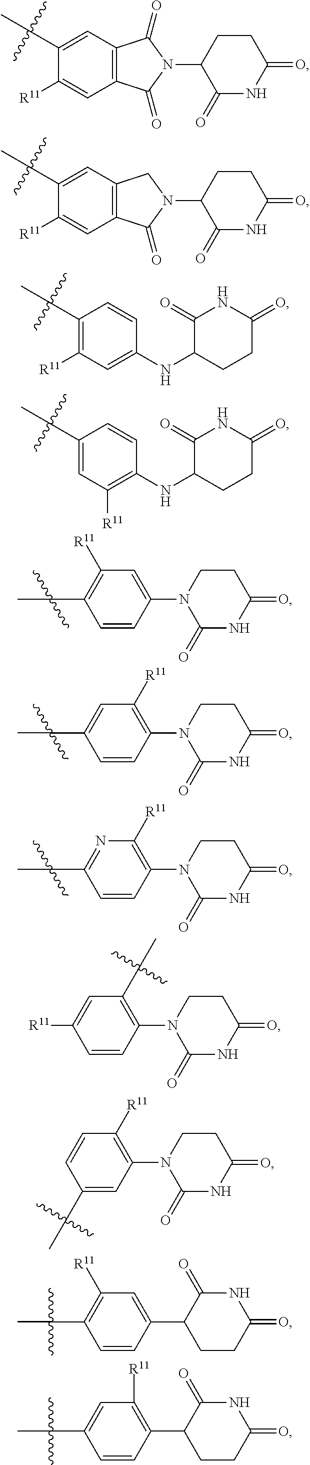

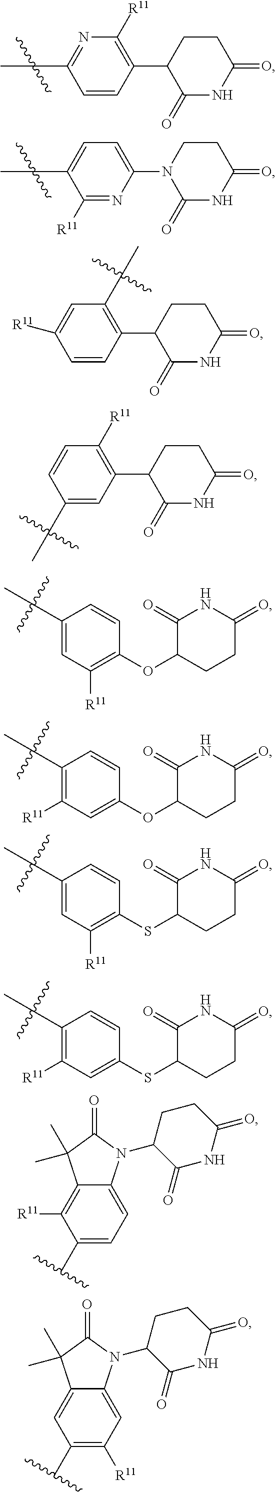

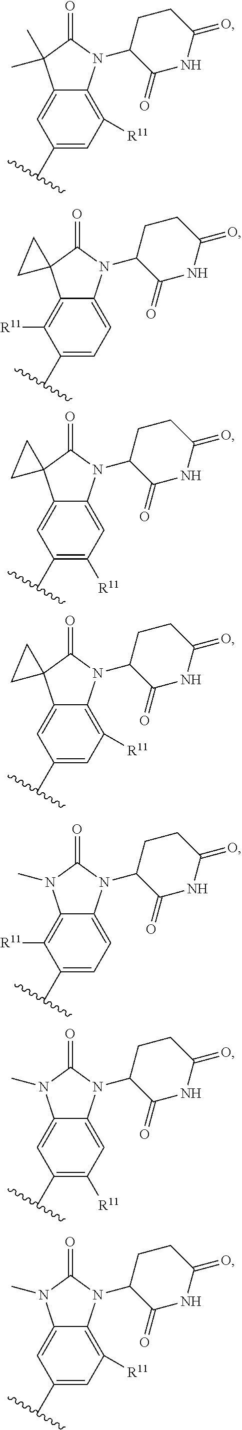

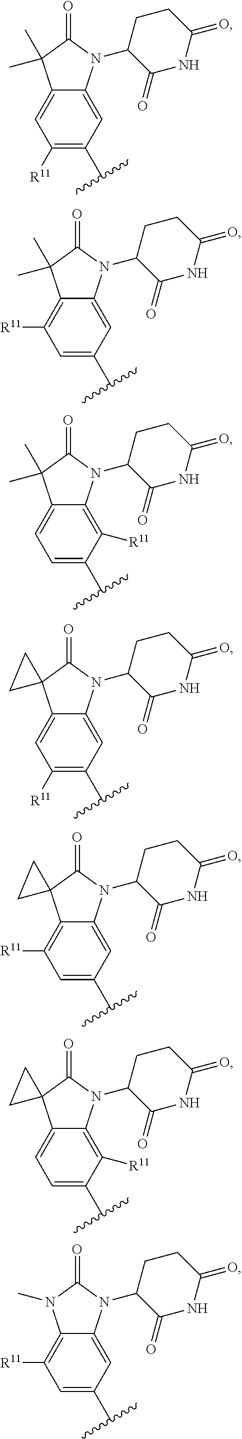

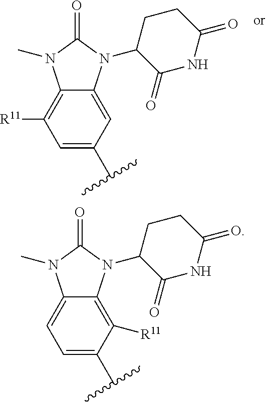

Aspect 11. The compound according to Aspect6, wherein [symbol] the moiety is selected from

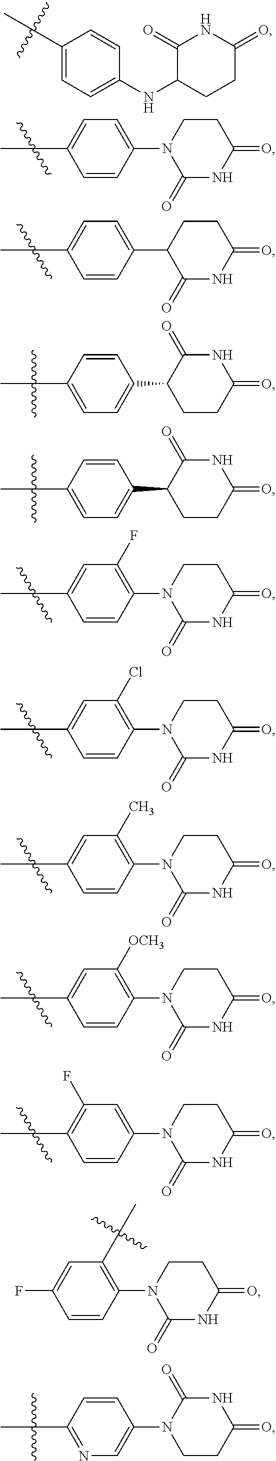

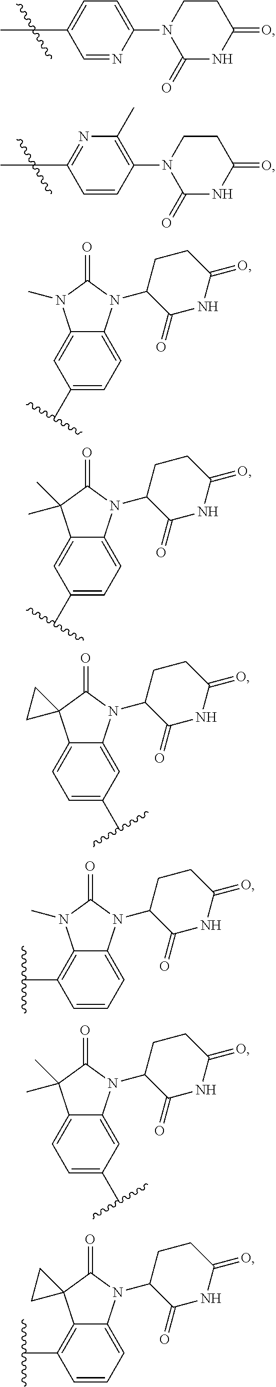

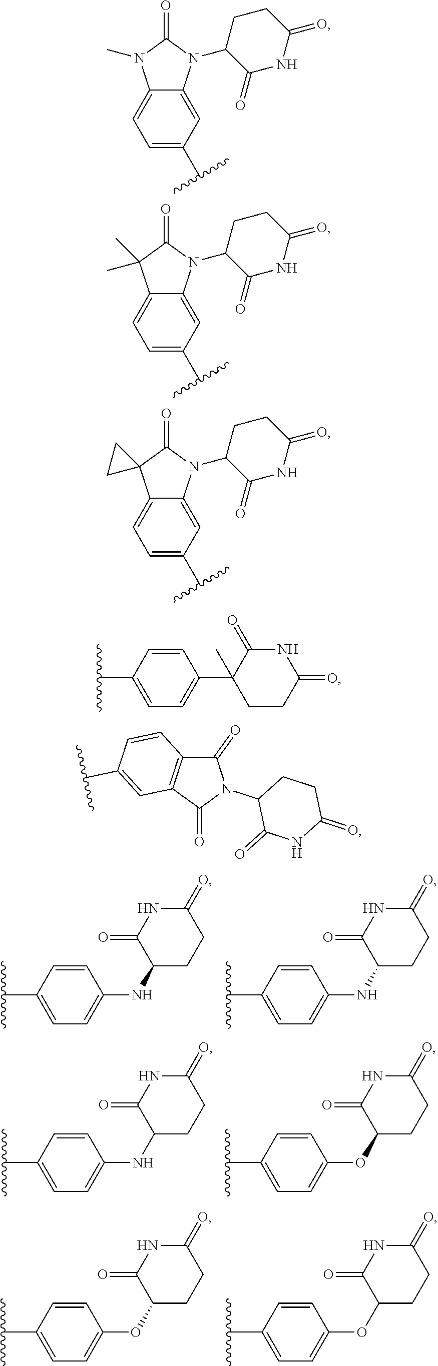

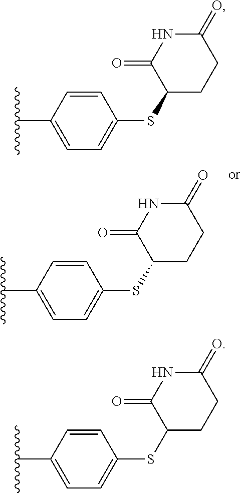 [SMILES: *c1ccc(S[C@@H]2CCC(=O)NC2=O)cc1 ; *c1ccc(S[C@H]2CCC(=O)NC2=O)cc1 ; *c1ccc(SC2CCC(=O)NC2=O)cc1]

Aspect 12. The compound according to Aspect1, wherein L^{a}, L^{b} and L^{c} are each independently selected from a single bond, —O—, —S—, —C(O)—, —(CR^{c}R^{d})ₙ— or —NR^{c}—;
n is 1, 2, 3, 4, 5 or 6;
R^{c} and R^{d} are each independently hydrogen, methyl, ethyl, propyl, butyl, pentyl, hexyl, heptyl, octyl, —C₂₋₈ alkenyl, —C₂₋₈alkynyl, cyclopropyl, cyclobutyl, cyclopentyl or cyclohexyl, cycloheptyl, cyclooctyl, heterocyclyl, aryl, or heteroaryl; or
R^{c} and R^{d} together with the atom(s) to which they are attached, form a 3-, 4-, 5-, 6-, 7-, 8-, 9-, 10-, 11- or 12-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN or —NO₂.

Aspect 13. The compound according to Aspect1, wherein L^{a}, L^{b} and L^{c} are each independently selected from a single bond, —O—, —CO—, —(CH₂)ₙ—, —[CH(CH₃)]ₙ—, —[C(CH₃)ₙ]— or —NH—; n is 1, 2, 3, 4 or 5.

Aspect 14. The compound according to Aspect1, R¹³ is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl.

Aspect 15. The compound according to Aspect1, wherein m1, m2, m3 and m4 are each independently selected from 0 or 1.

Aspect 16. The compound according to Aspect1, wherein p3 and p4 are each independently 0 or 1, R⁸ and R⁹ are each independently selected from hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl.

Aspect 17. The compound according to Aspect1, wherein the

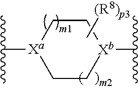

moiety is selected from

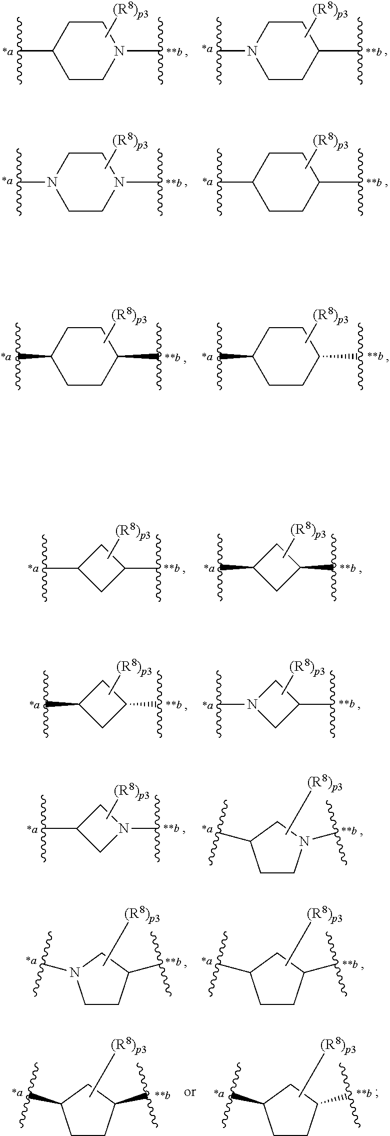

wherein *a refers to the position attached to the

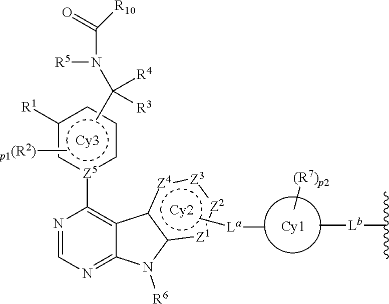

moiety, and **b refers to the position attached to

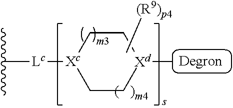

moiety.

Aspect 18. The compound according to Aspect1, wherein the

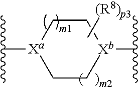

moiety is selected from

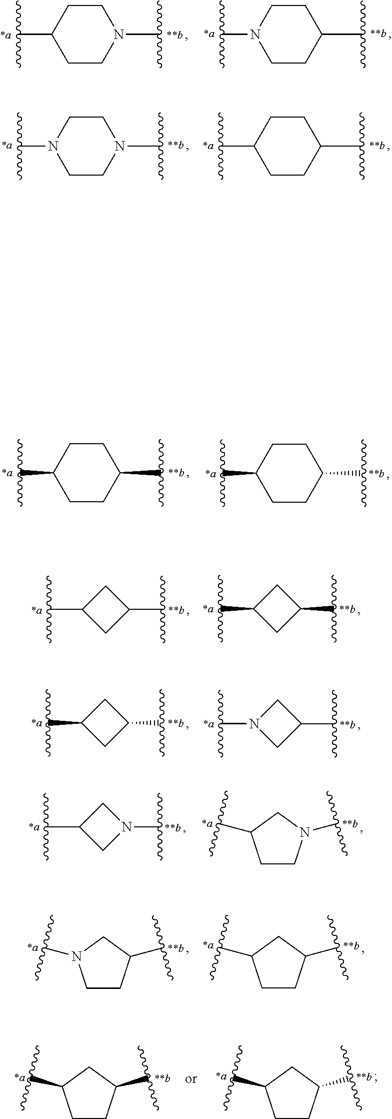

wherein *a refers to the position attached to the

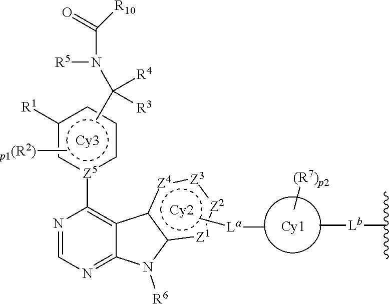

moiety, and **b refers to the position attached to

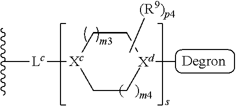

moiety.

Aspect 19. The compound according to Aspect1, wherein the

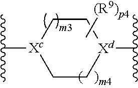

moiety is selected from

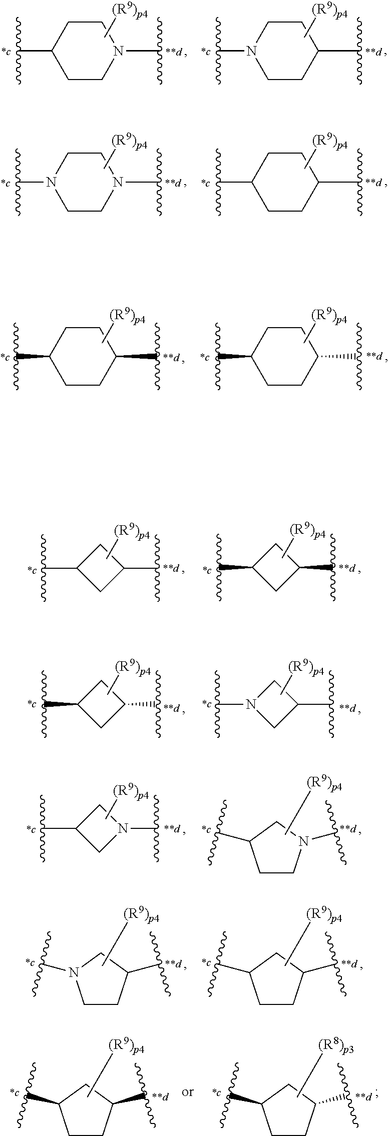

wherein *a refers to the position attached to the

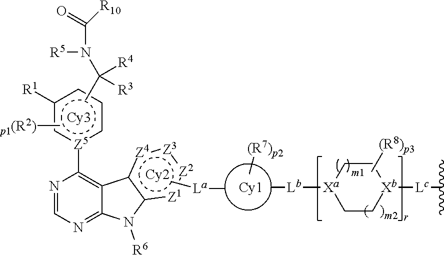

moiety, and **b refers to the position attached to

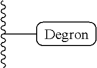

moiety.

Aspect 20. The compound according to Aspect1, wherein the

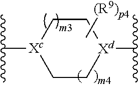

moiety is selected from

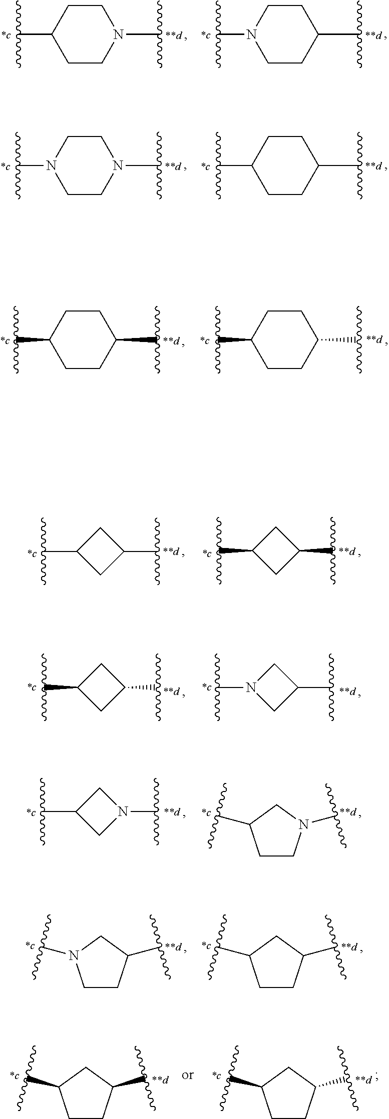

wherein *a refers to the position attached to the

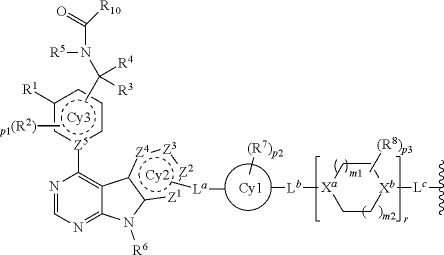

moiety, and **b refers to the position attached to

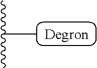

moiety.

Aspect 21. The compound according to Aspect1, wherein the

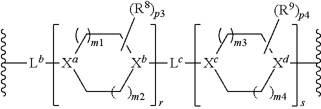

moiety is selected from —CH₂—, —(CH₂)₂—, —(CH₂)₃—, —(CH₂)₄,

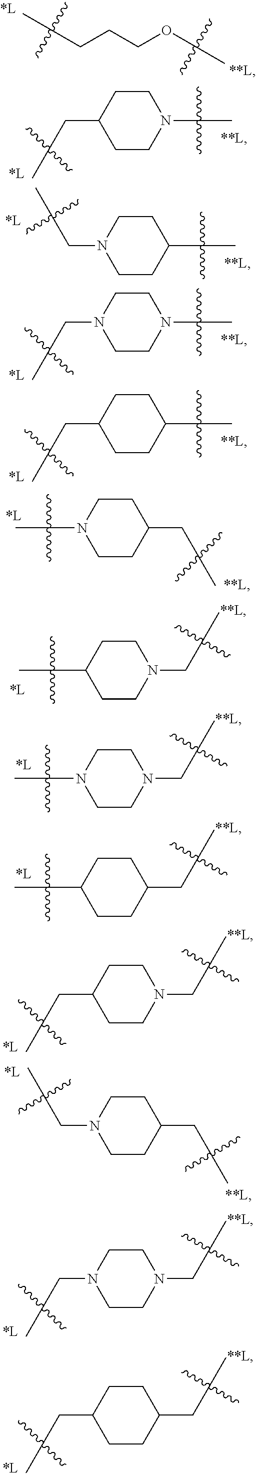

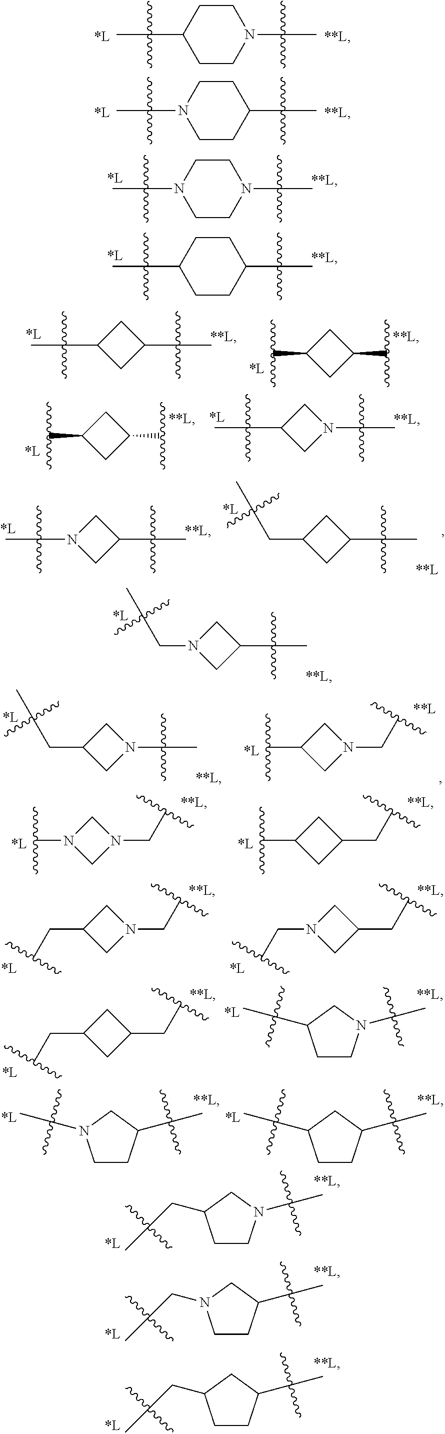

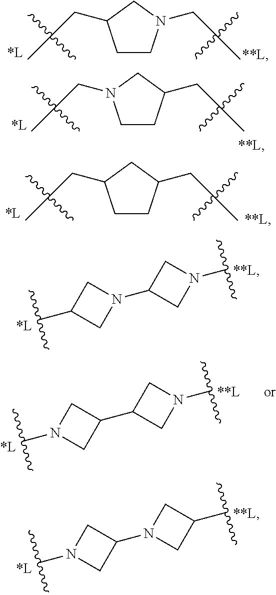

*L refers to the position attached to

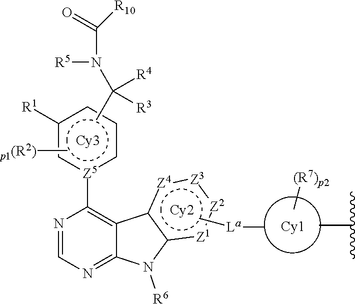

moiety, and **L refers to the position attached

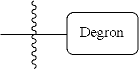

moiety.

Aspect 22. The compound according to Aspect1, wherein Z¹ and Z⁴ are each independently CR^{za} or N; R^{za} is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl, —C₂₋₈alkenyl, —C₂₋₈alkynyl, heterocyclyl, aryl, heteroaryl, —CN.

Aspect 23. The compound according to Aspect1, wherein one of Z² and Z³ is C, and connects to Cy1; and the other one of Z² and Z³ is CR^{za} or N; R^{za} is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl, —C₂₋₈alkenyl, —C₂₋₈alkynyl, heterocyclyl, aryl, heteroaryl, —CN.

Aspect 24. The compound according to Aspect1, wherein at most two of Z¹, Z², Z³ and Z⁴ are N.

Aspect 25. The compound according to Aspect1, wherein at most one of Z¹, Z², Z³ and Z⁴ is N.

Aspect 26. The compound according to Aspect1, wherein R¹ is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃ or —CH₂CH₂CF₃.

Aspect 27. The compound according to Aspect1, wherein R¹ and R³, together with the atom(s) to which they are attached, form a 3-, 4-, 5-, 6-, 7- or 8-membered ring, said ring comprising 0, 1 or 2 additional N atom as ring member(s), said ring is optionally substituted with at least one substituent independently selected from —F, —Cl, —Br, —I, —OH, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or —CH₂OH.

Aspect 28. The compound according to Aspect1, wherein R² is H, —F, —Cl, —Br, —I, methyl, ethyl or propyl, and p1 is 0, 1, or 2.

Aspect 29. The compound according to Aspect1, wherein R³ and R⁴ are each independently —H, methyl, ethyl, propyl, butyl, pentyl, hexyl, cyclopropyl, cyclobutyl, cyclopentyl or cyclohexyl.

Aspect 30. The compound according to Aspect1, wherein the

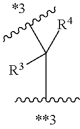

moiety is

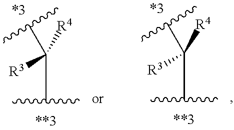

wherein *3 refers to the position attached to the

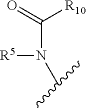

moiety, and **3 refers to the position attached to

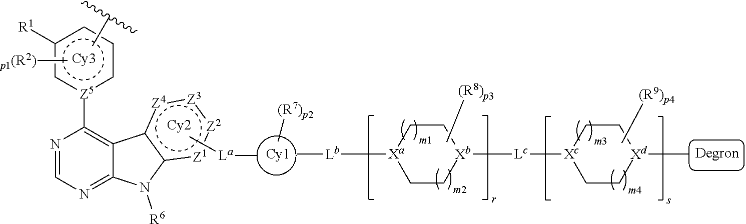

moiety.

Aspect 31. The compound according to Aspect1, wherein R⁵ and R⁶ are each independently —H, methyl, ethyl, propyl.

Aspect 32. The compound according to Aspect1, wherein R¹⁰ is

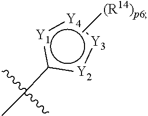

Y¹, Y², Y³ and Y⁴ are selected from CH, O, S or N; R¹⁴ is selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₁₋₈alkoxy, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{a}, R^{b}, and R^{c} are each independently hydrogen, —C₁₋₈alkyl, —C₂₋₈alkenyl, —C₂₋₈alkynyl, cycloalkyl, heterocyclyl, aryl, or heteroaryl; and p6 is 0, 1, 2, 3 or 4.

Aspect 33. The compound according to Aspect25, wherein Y¹ is CH, S, N or O; Y² is CH, S, O or N; Y³ is CH, O, S or N; and Y⁴ is CH, O, S or N.

Aspect 34. The compound according to Aspect25, wherein

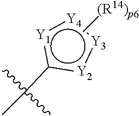

is selected from

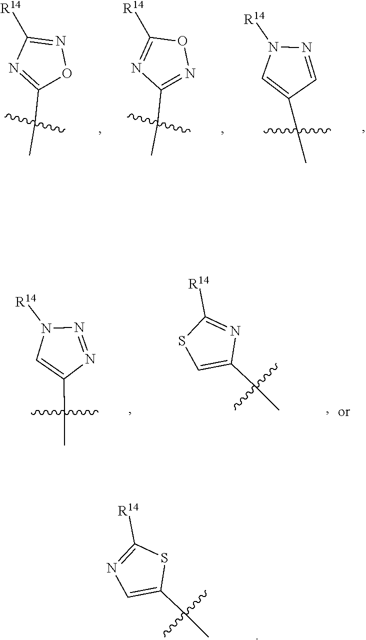

Aspect 35. The compound according to Aspect25, wherein, R¹⁴ is selected from —H, —F, —Cl, —Br, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃, —CH₂CH₂CF₃, each of said —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈ alkynyl is optionally substituted with at least one halogen, hydroxy, -haloC₁₋₈alkyl, —C₁₋₈alkyoxy, cycloalkyl, heterocyclyl, aryl, or heteroaryl.

Aspect 36. The compound according to Aspect25, wherein, R¹⁴ is selected from

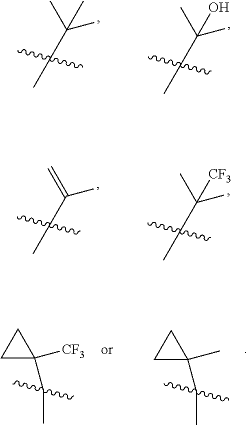

Aspect37. The compound according to Aspect1, wherein the compound is selected from

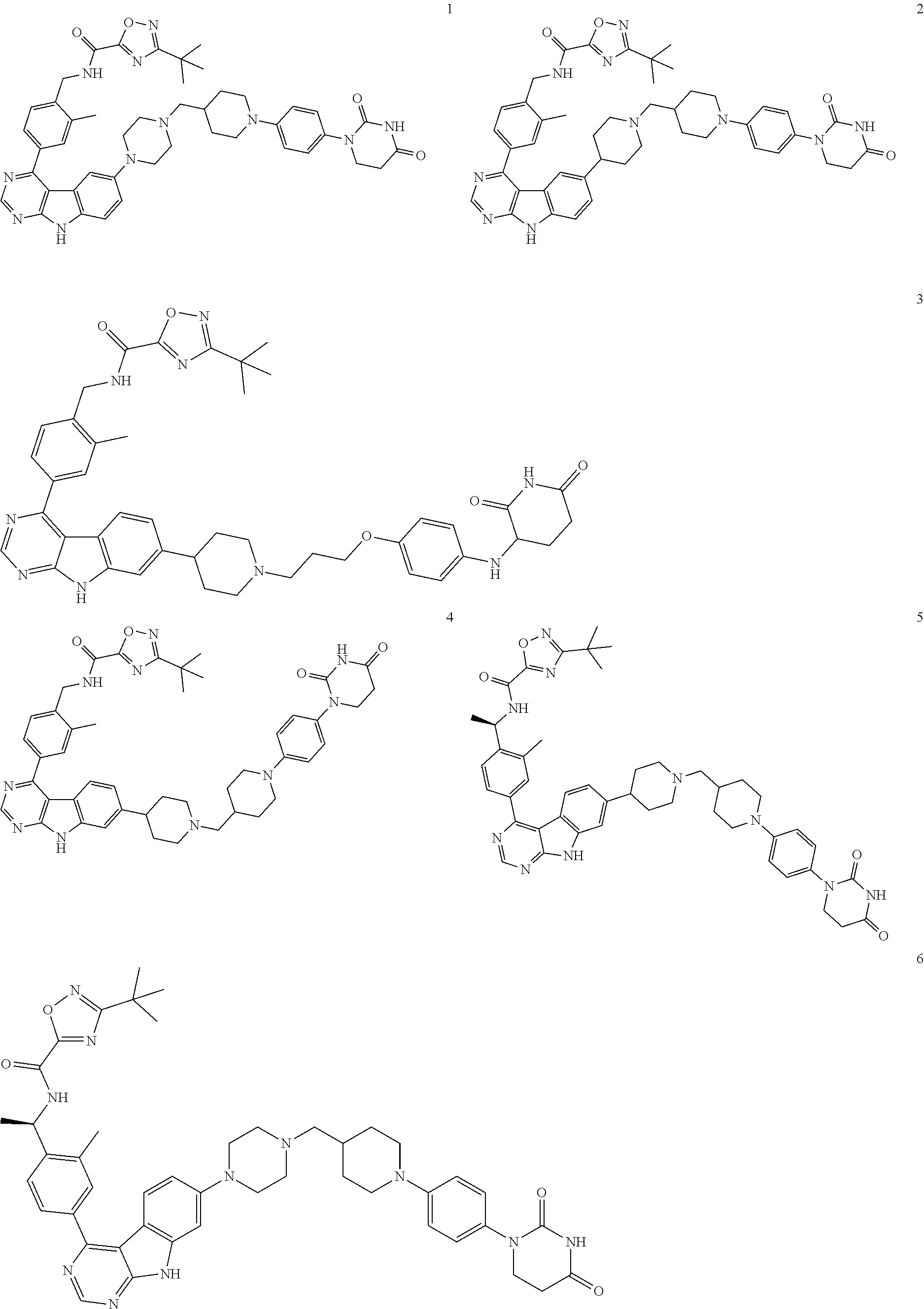

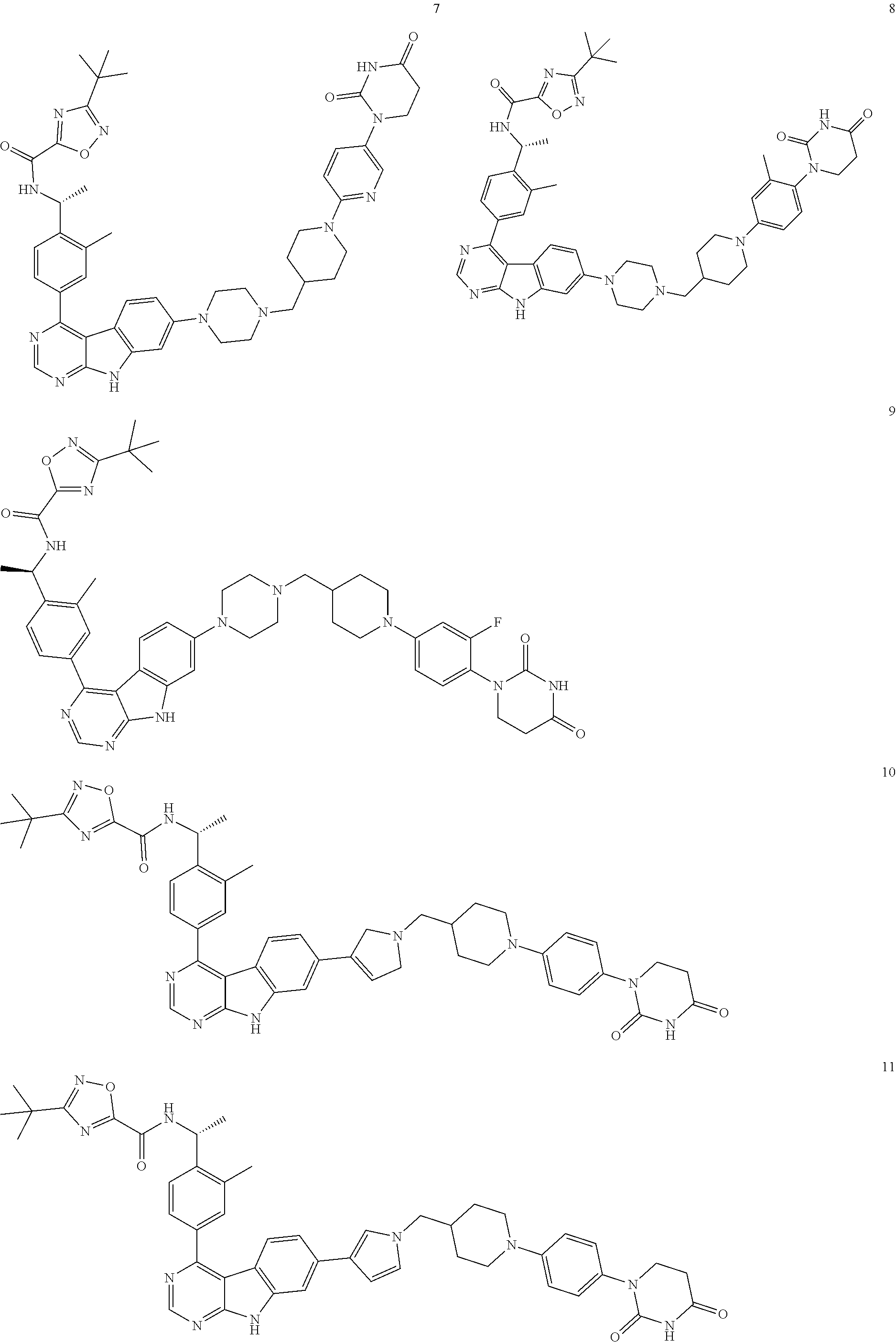

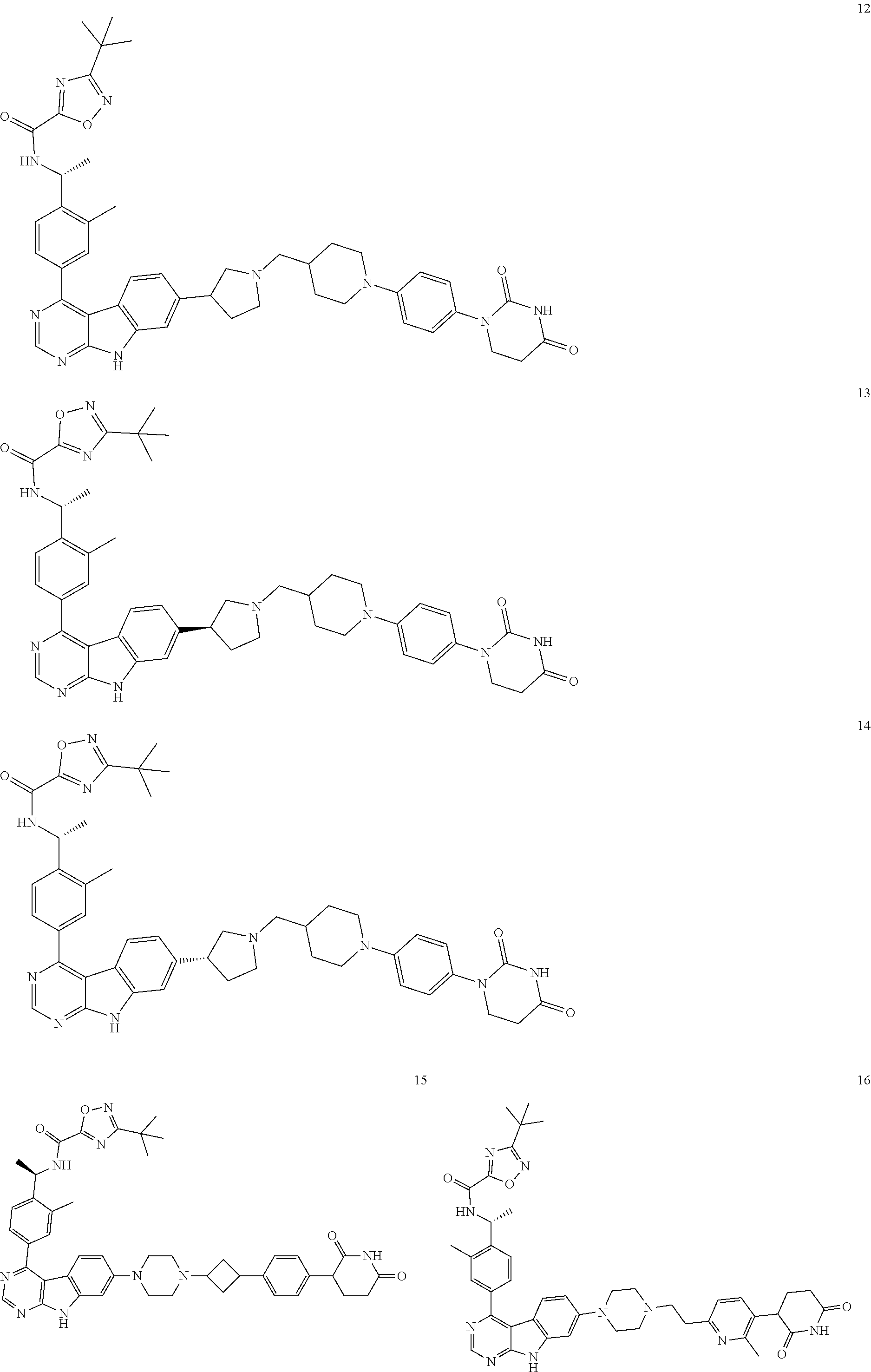

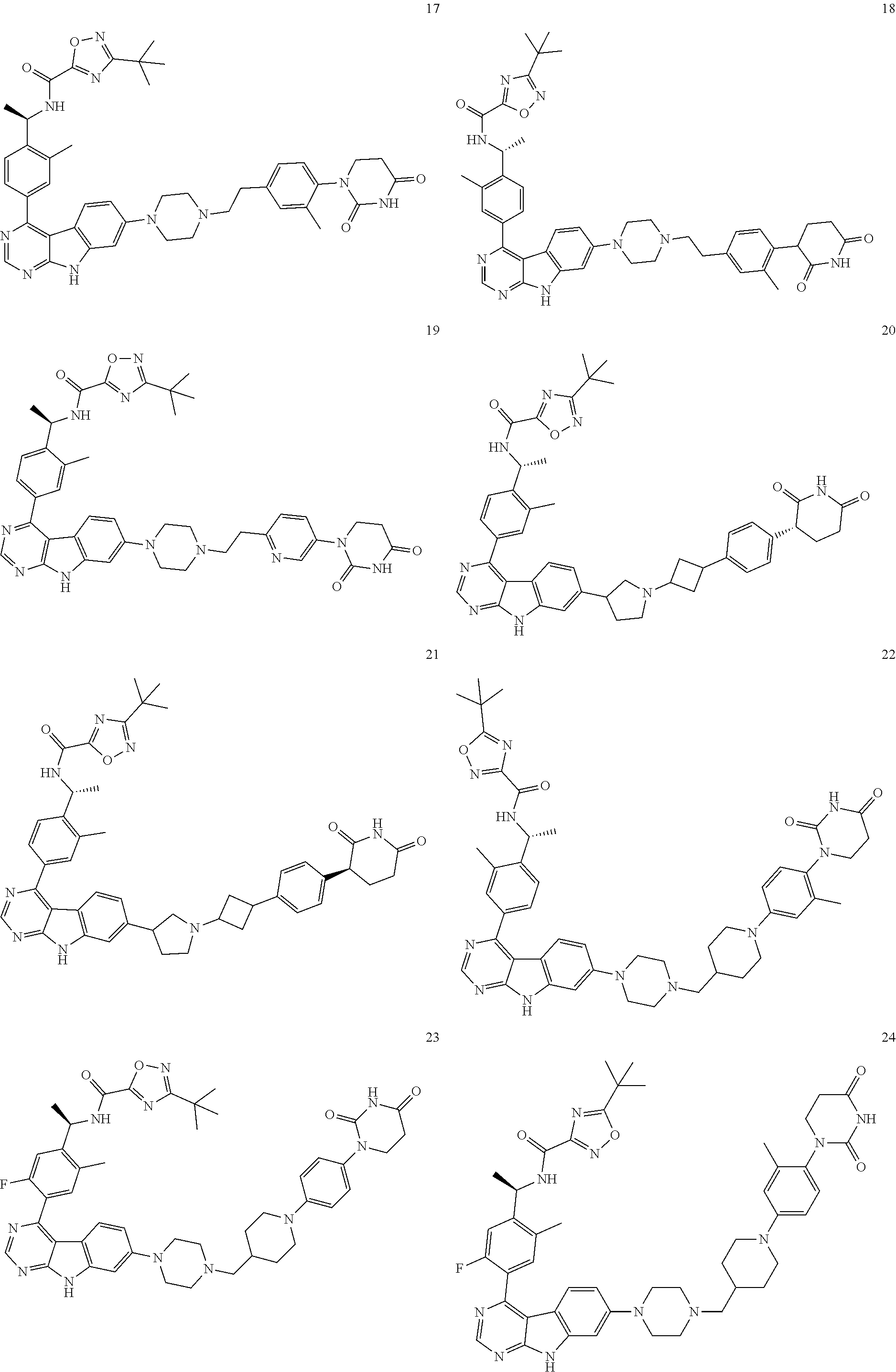

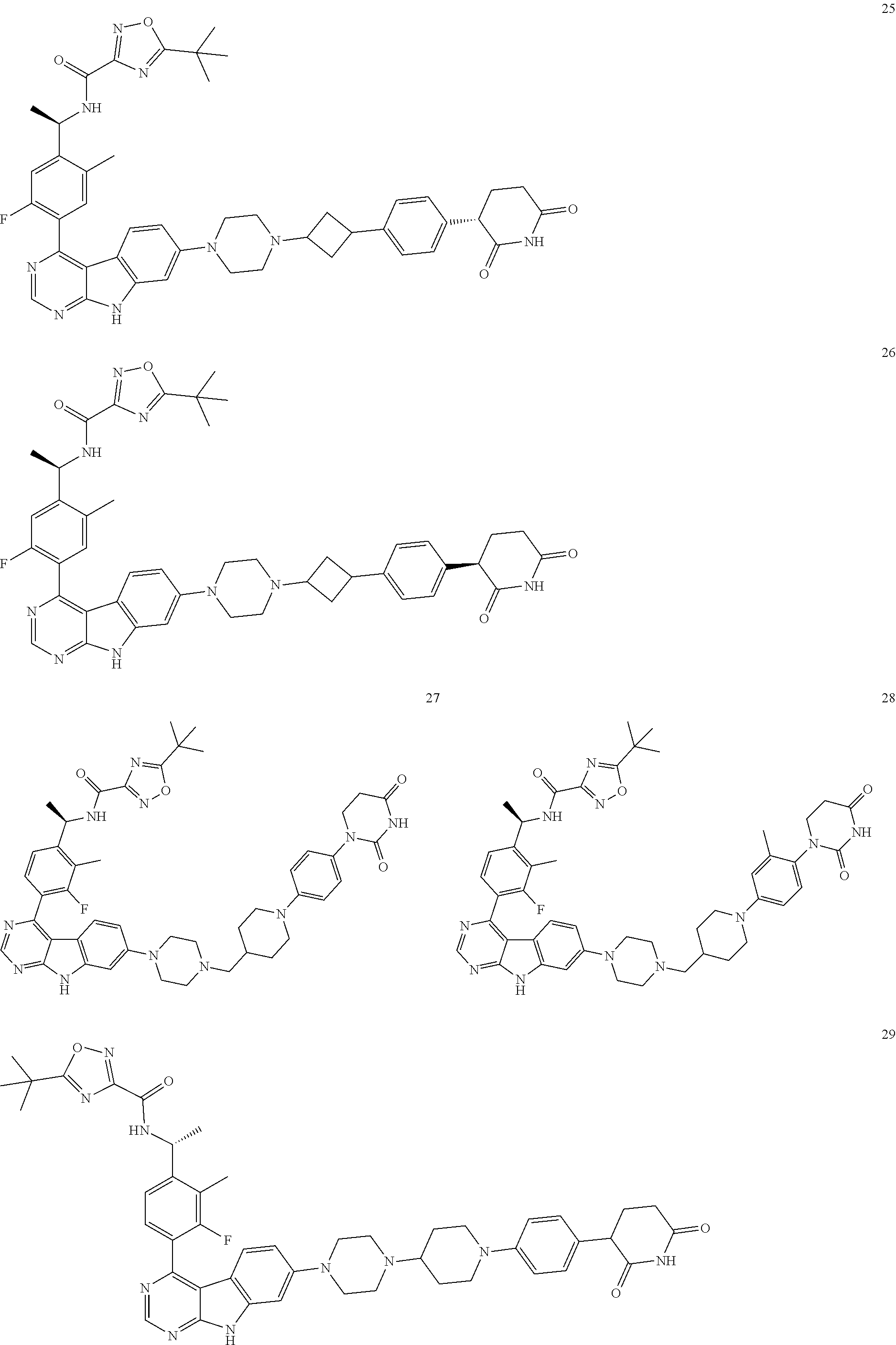

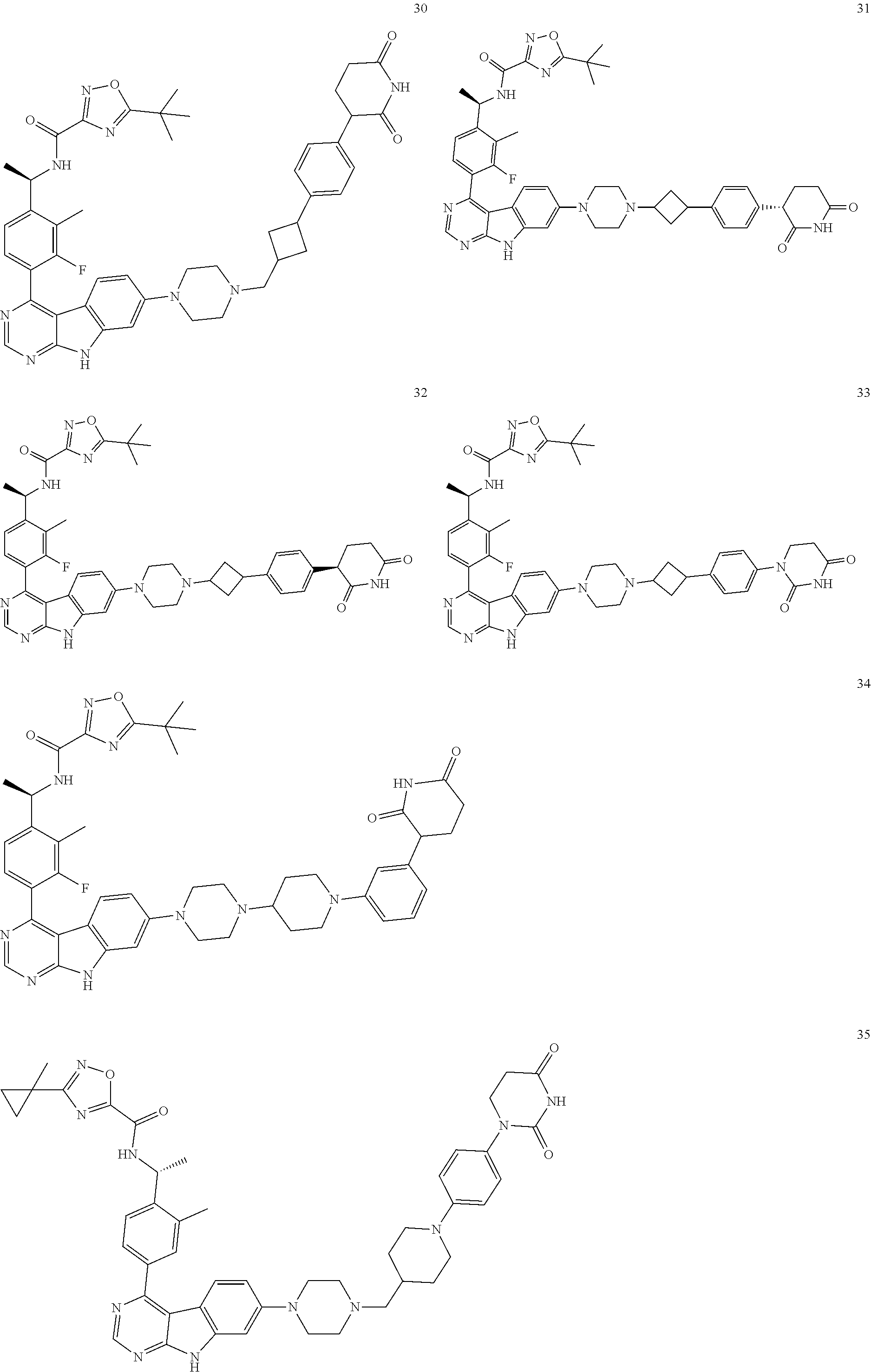

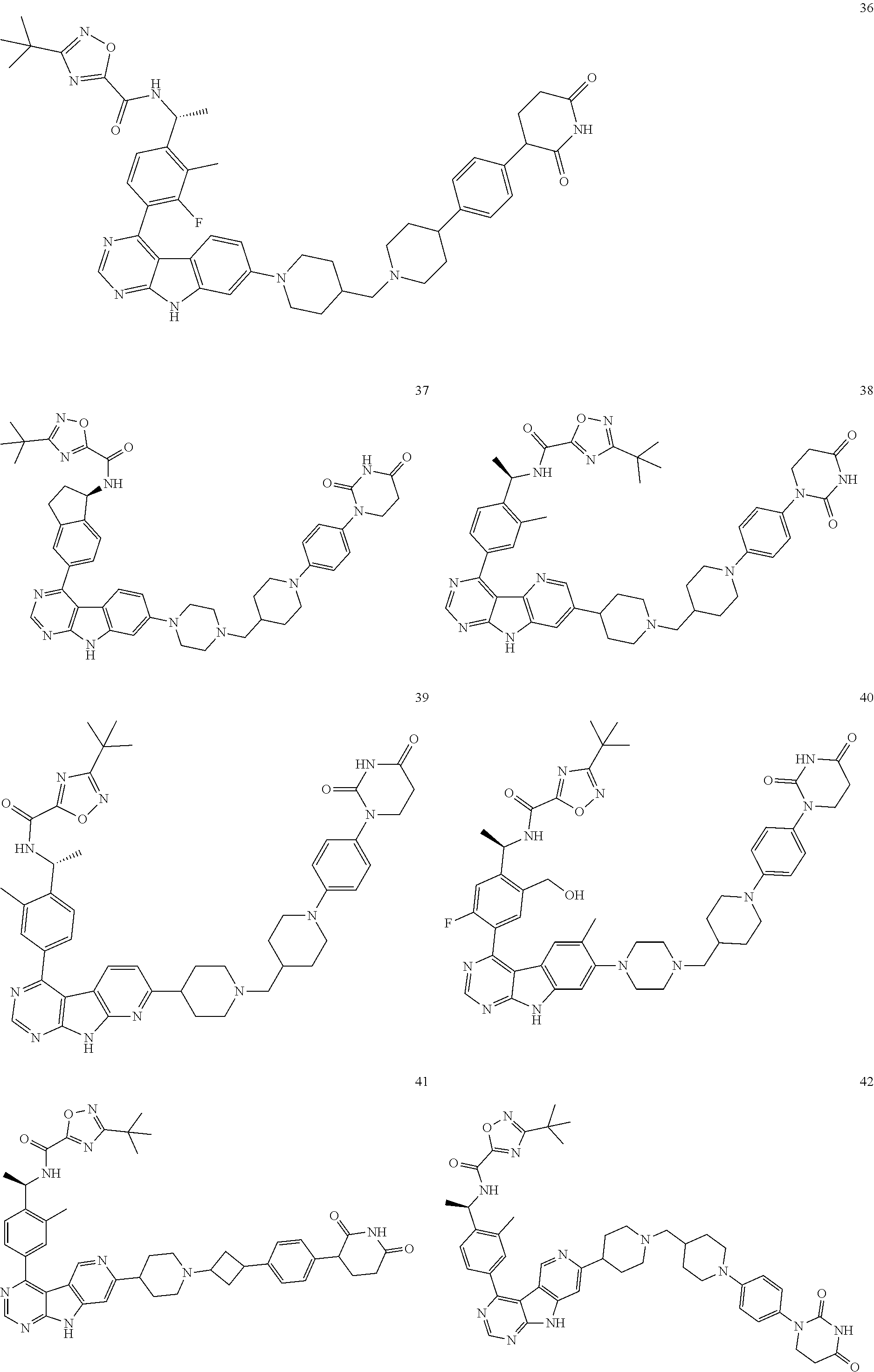

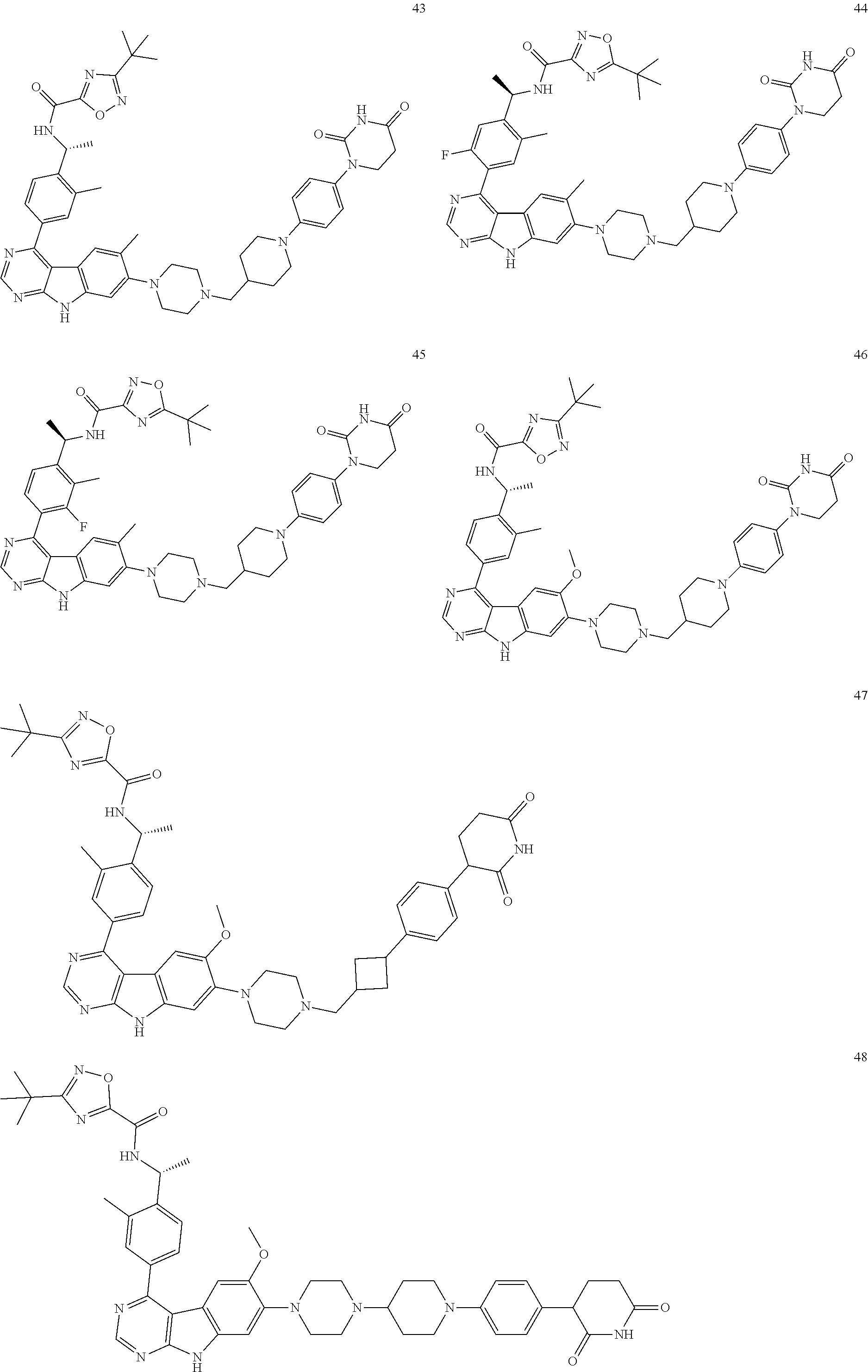

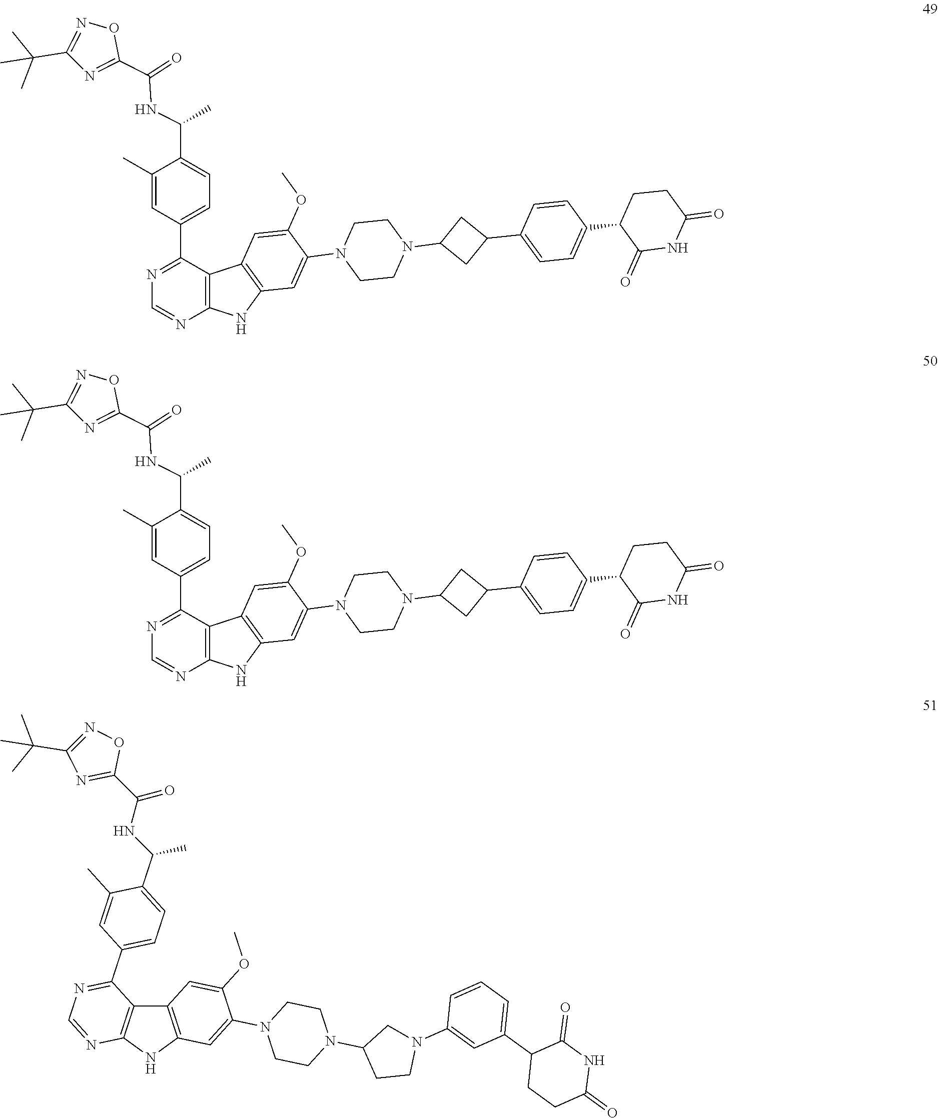 [SMILES: COc1cc2c(cc1N1CCN(C3CC(c4ccc([C@@H]5CCC(=O)NC5=O)cc4)C3)CC1)[nH]c1ncnc(-c3ccc([C@@H](C)NC(=O)c4nc(C(C)(C)C)no4)c(C)c3)c12 ; COc1cc2c(cc1N1CCN(C3CC(c4ccc([C@@H]5CCC(=O)NC5=O)cc4)C3)CC1)[nH]c1ncnc(-c3ccc([C@@H](C)NC(=O)c4nc(C(C)(C)C)no4)c(C)c3)c12 ; COc1cc2c(cc1N1CCN(C3CCN(c4cccc(C5CCC(=O)NC5=O)c4)C3)CC1)[nH]c1ncnc(-c3ccc([C@@H](C)NC(=O)c4nc(C(C)(C)C)no4)c(C)c3)c12]

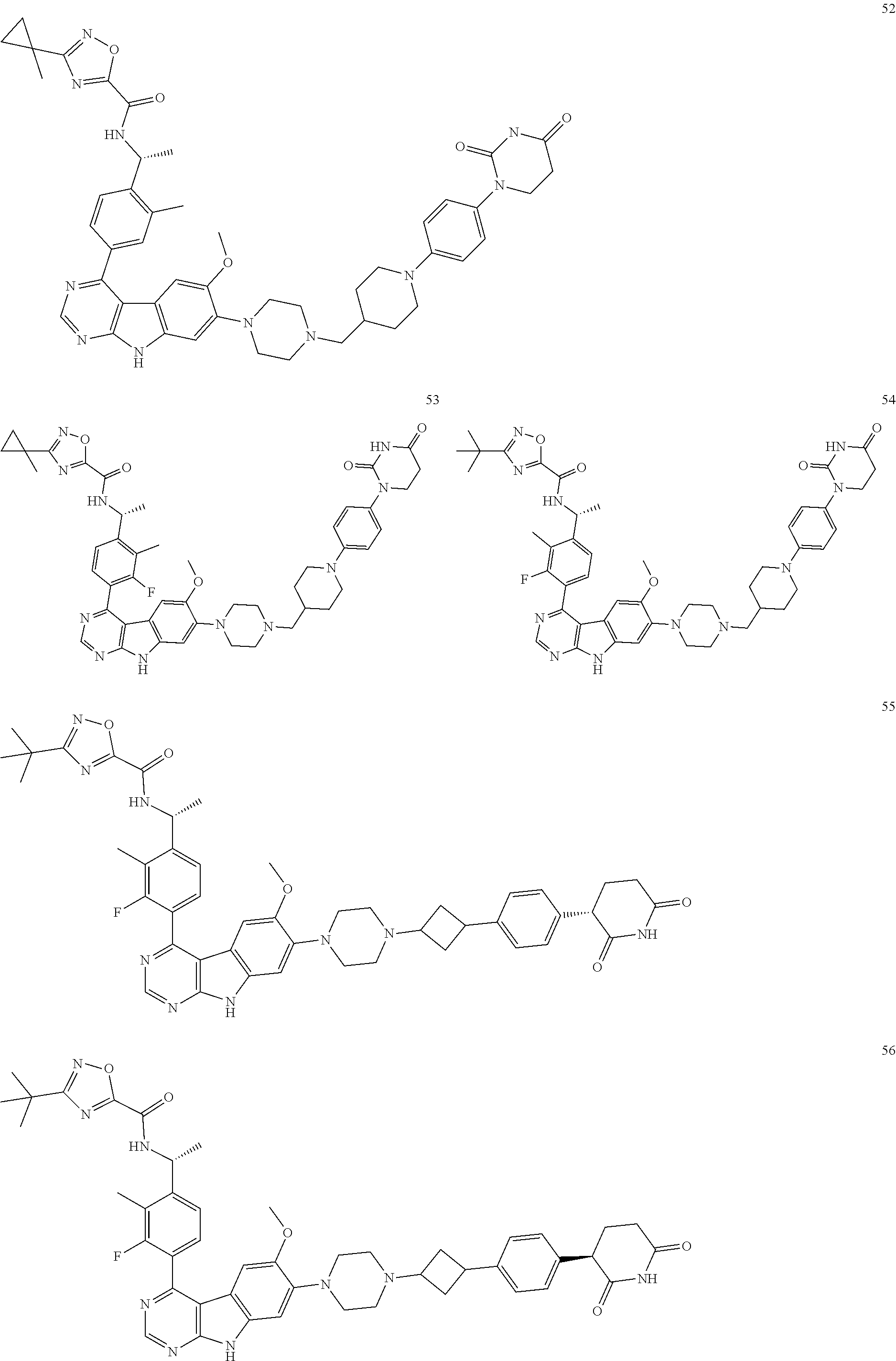

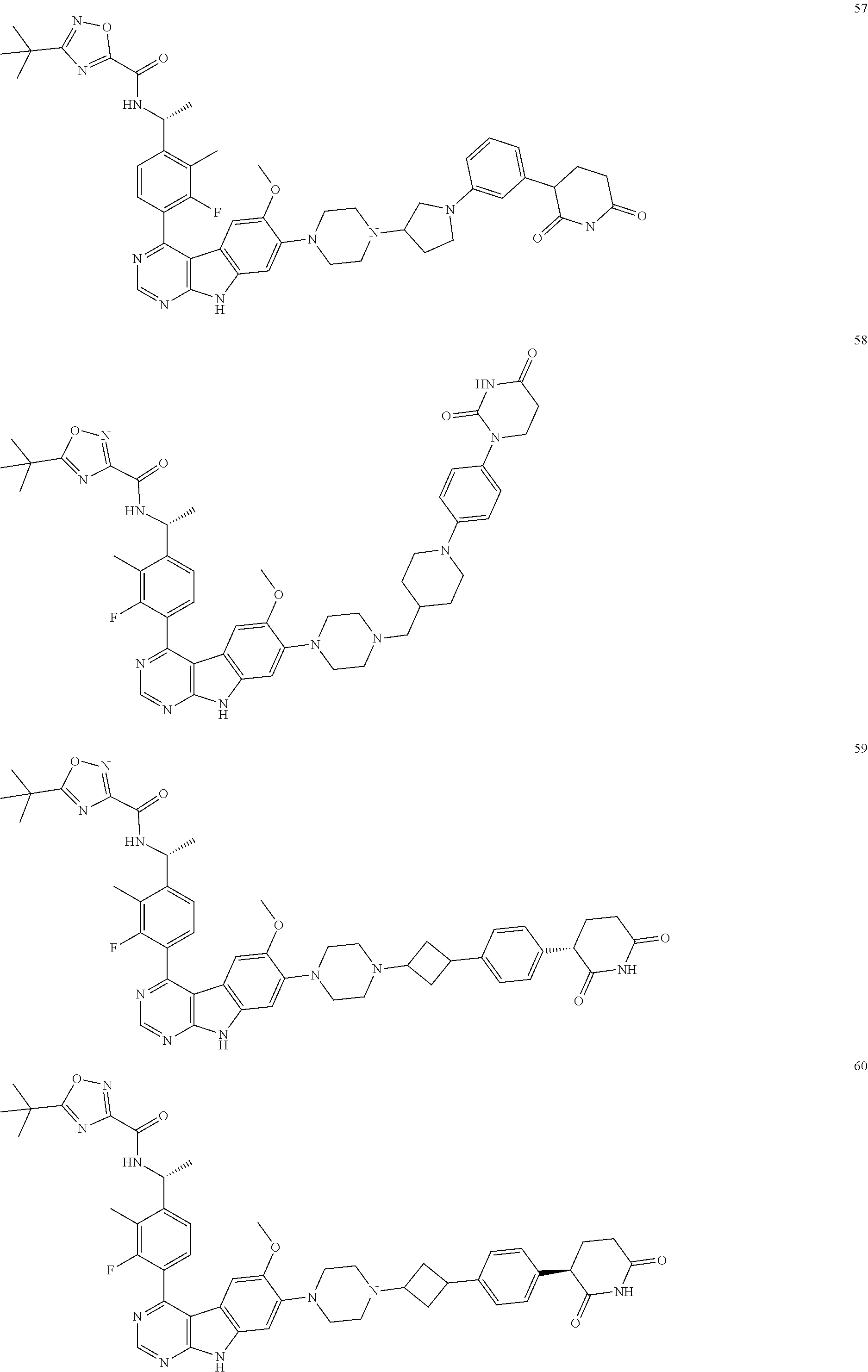

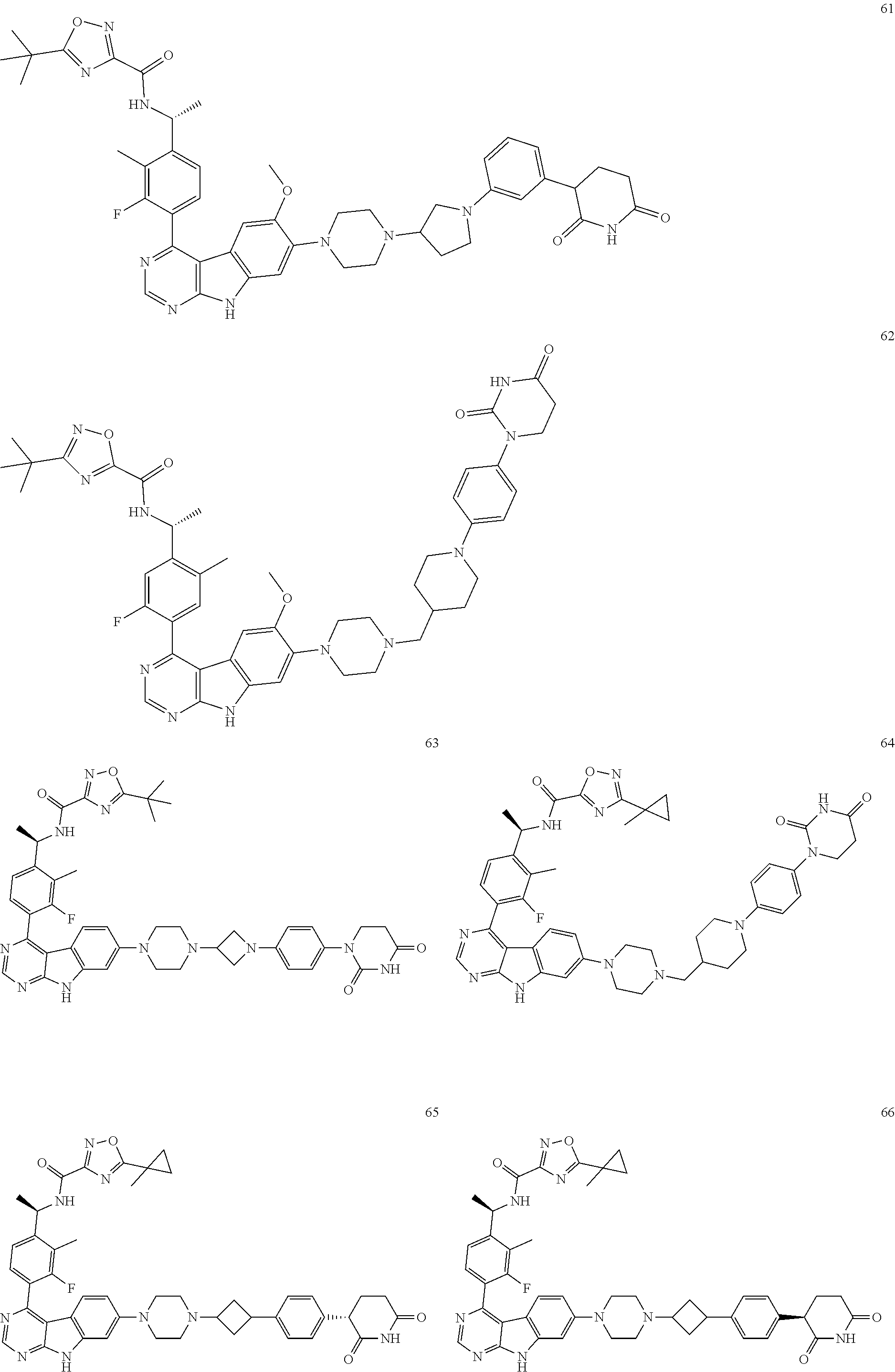

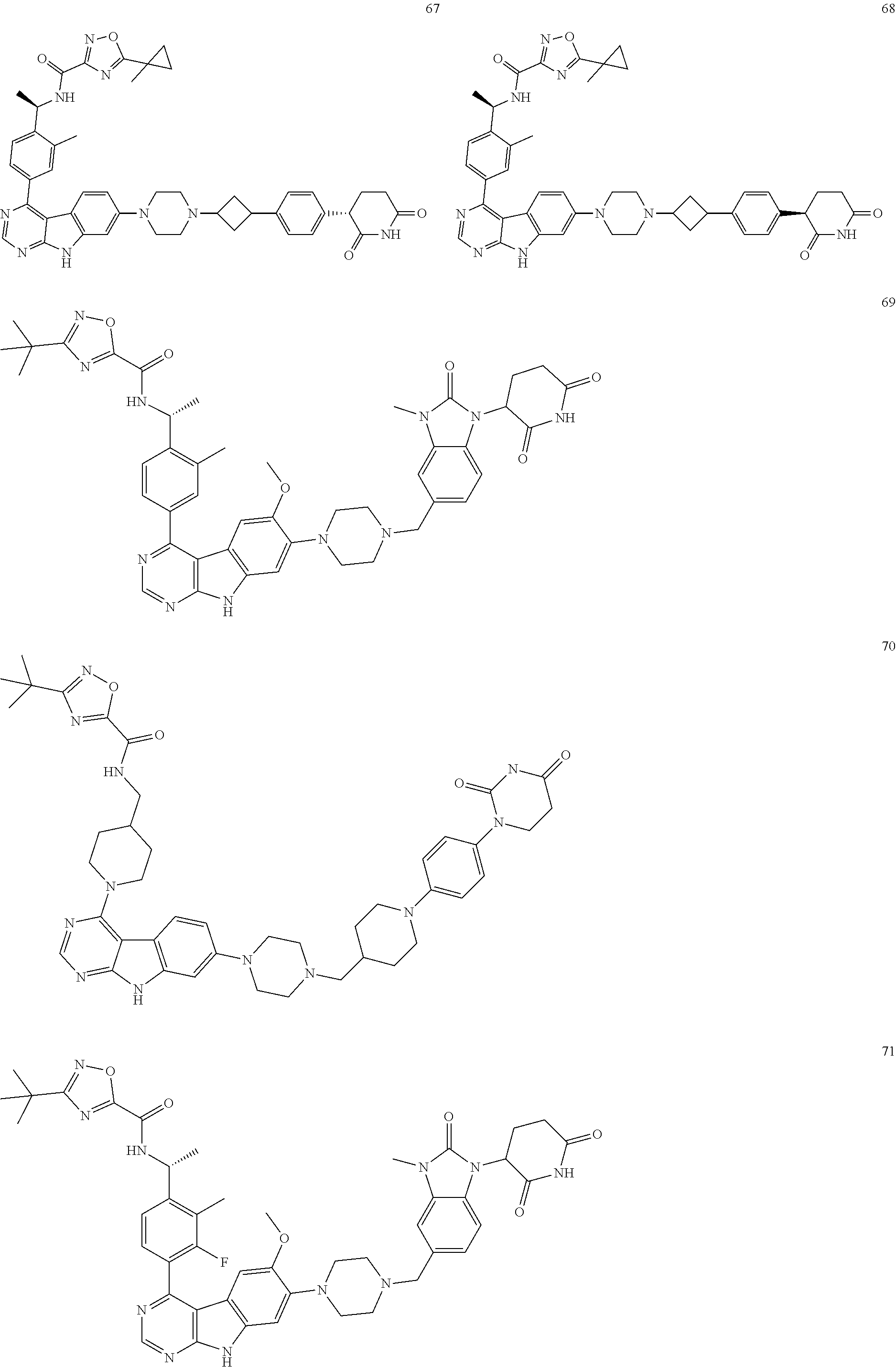

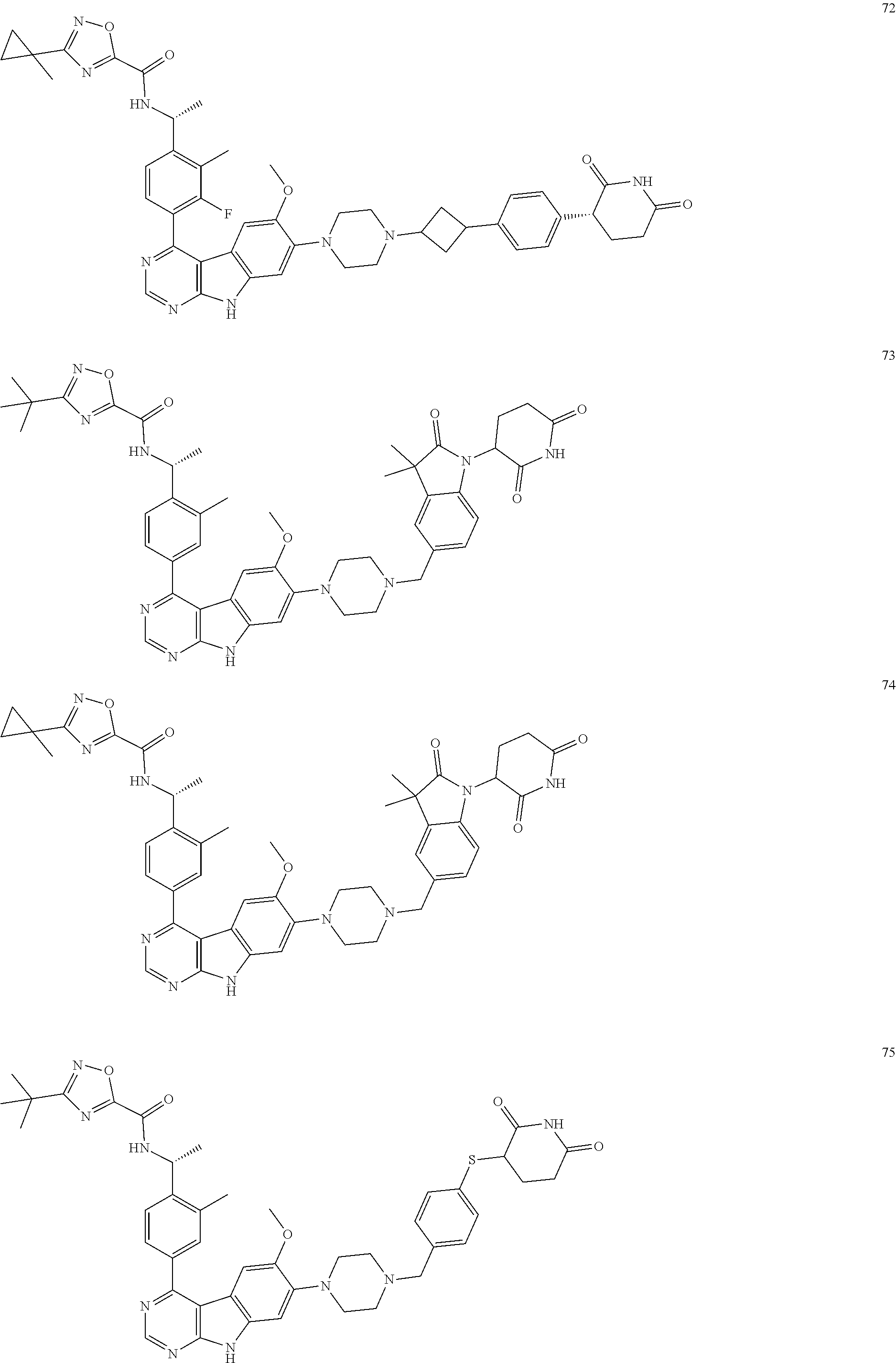 [SMILES: COc1cc2c(cc1N1CCN(C3CC(c4ccc([C@H]5CCC(=O)NC5=O)cc4)C3)CC1)[nH]c1ncnc(-c3ccc([C@@H](C)NC(=O)c4nc(C5(C)CC5)no4)c(C)c3F)c12 ; COc1cc2c(cc1N1CCN(Cc3ccc4c(c3)C(C)(C)C(=O)N4C3CCC(=O)NC3=O)CC1)[nH]c1ncnc(-c3ccc([C@@H](C)NC(=O)c4nc(C(C)(C)C)no4)c(C)c3)c12 ; COc1cc2c(cc1N1CCN(Cc3ccc4c(c3)C(C)(C)C(=O)N4C3CCC(=O)NC3=O)CC1)[nH]c1ncnc(-c3ccc([C@@H](C)NC(=O)c4nc(C5(C)CC5)no4)c(C)c3)c12 ; COc1cc2c(cc1N1CCN(Cc3ccc(SC4CCC(=O)NC4=O)cc3)CC1)[nH]c1ncnc(-c3ccc([C@@H](C)NC(=O)c4nc(C(C)(C)C)no4)c(C)c3)c12]

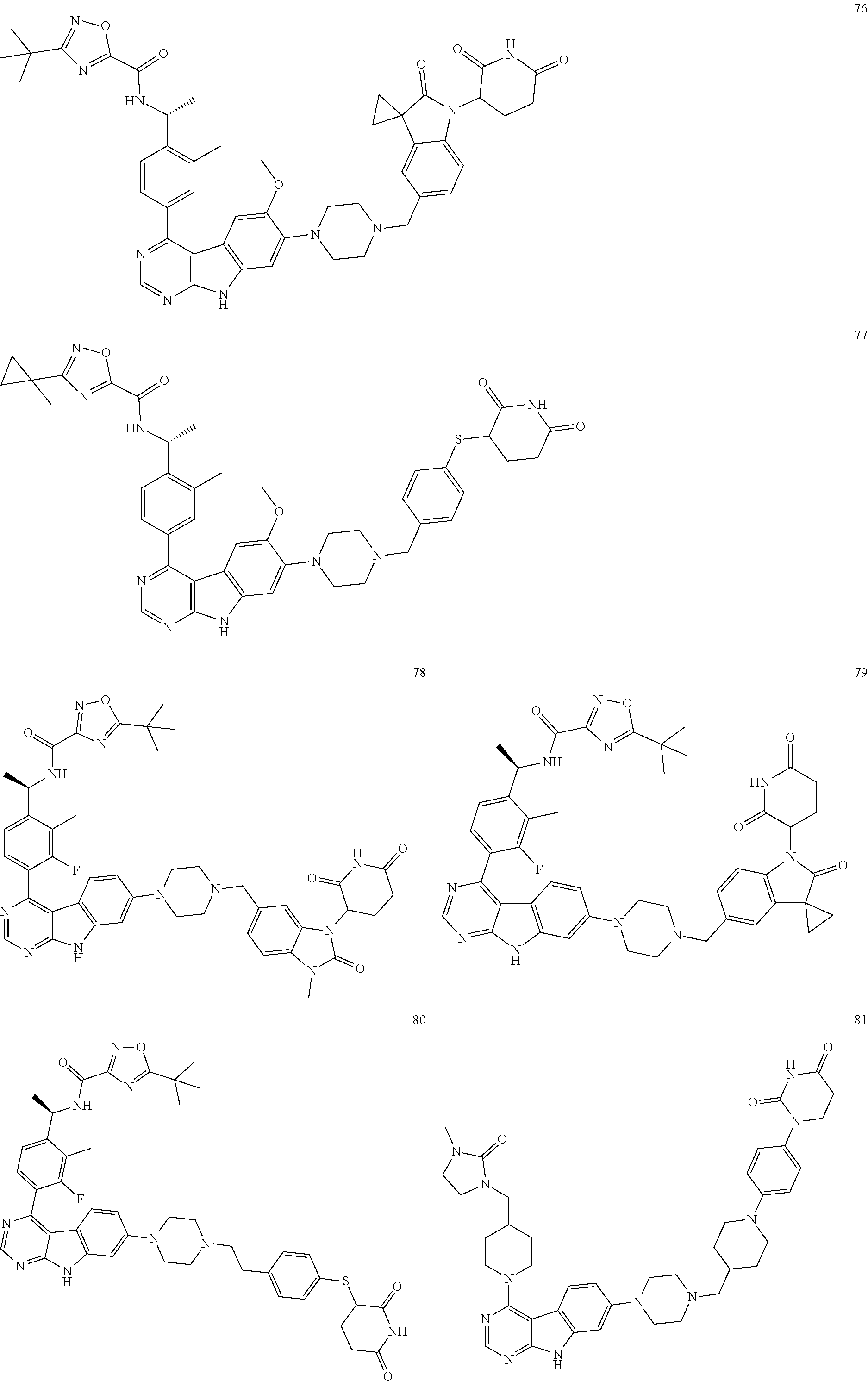

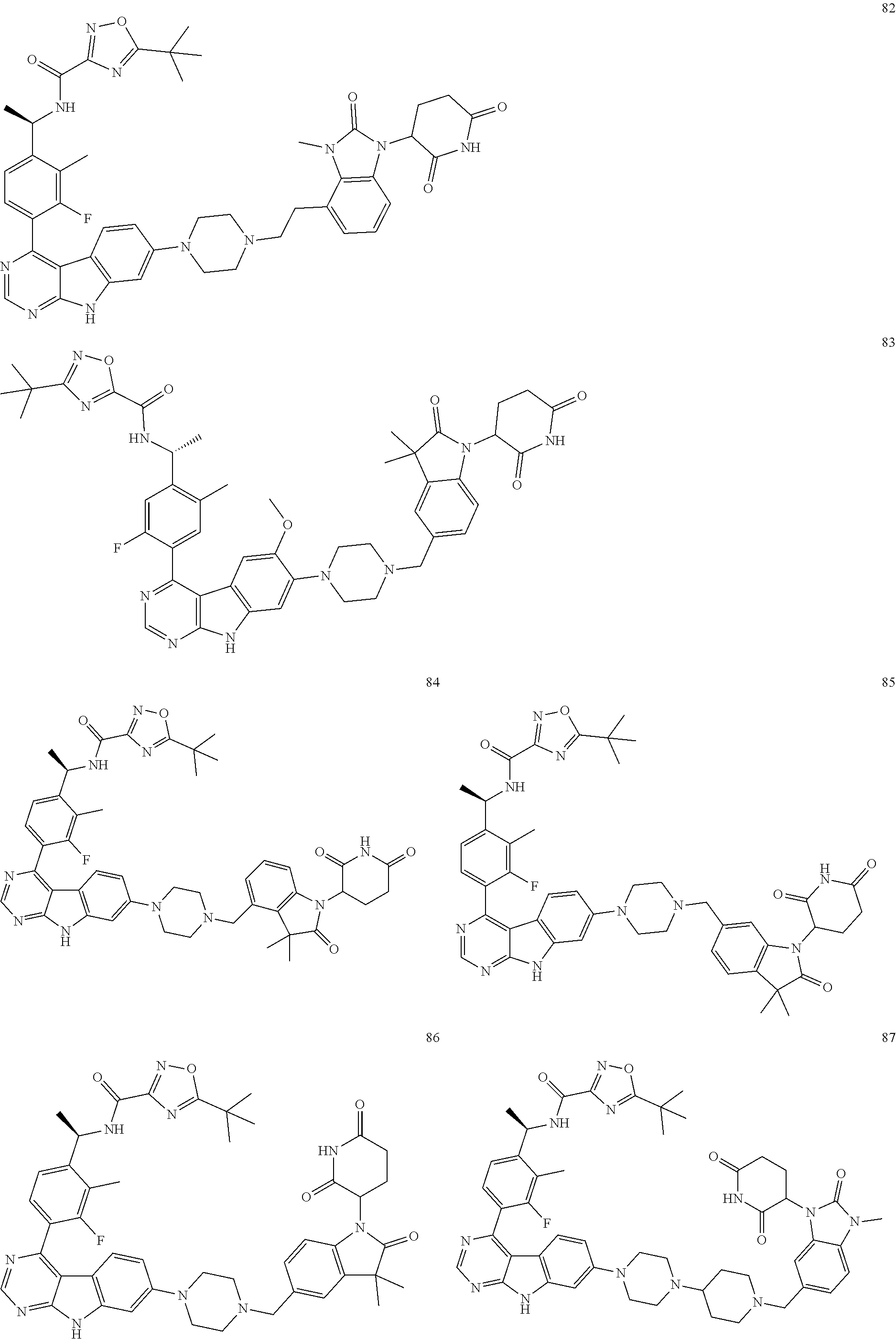

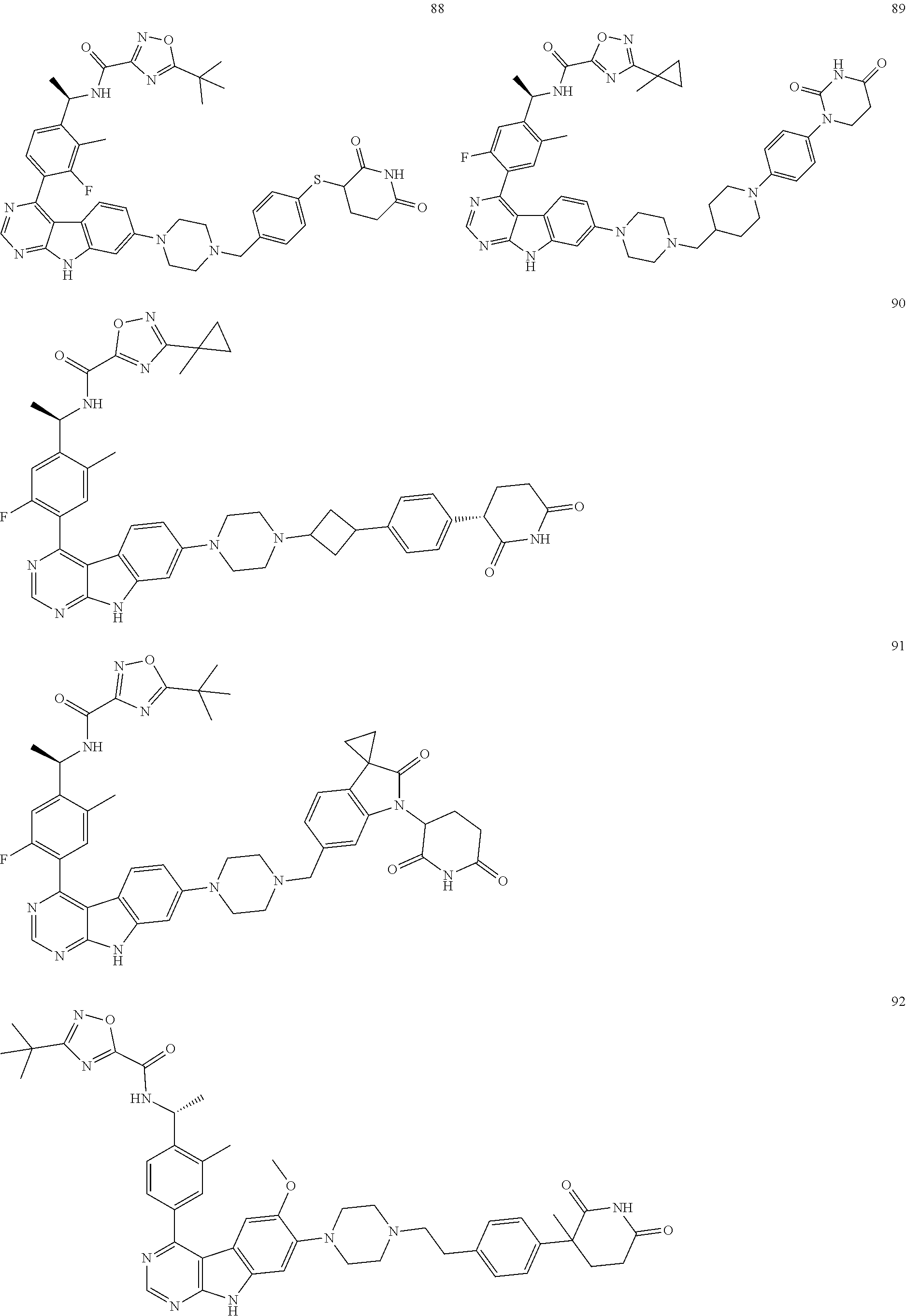

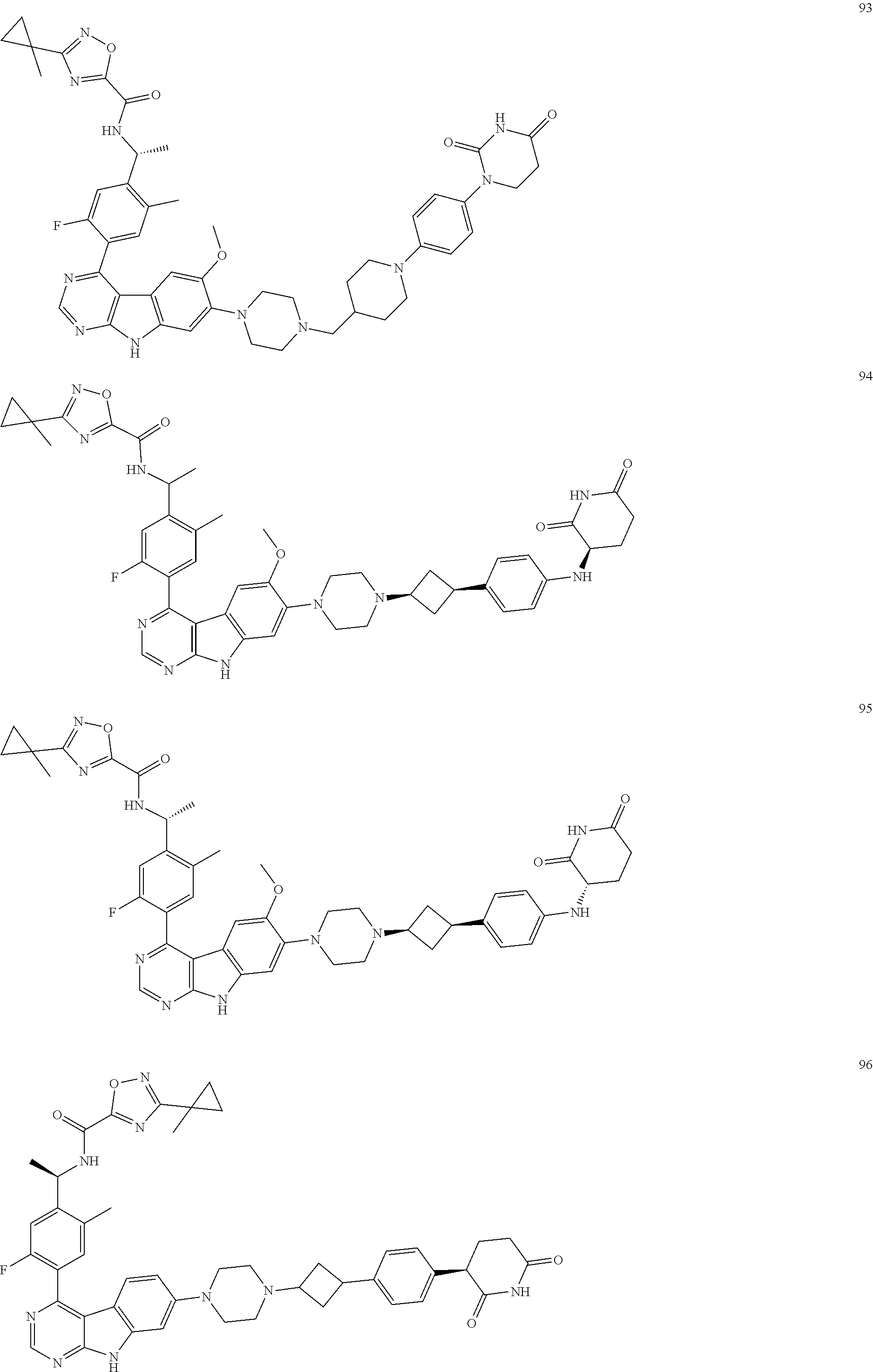

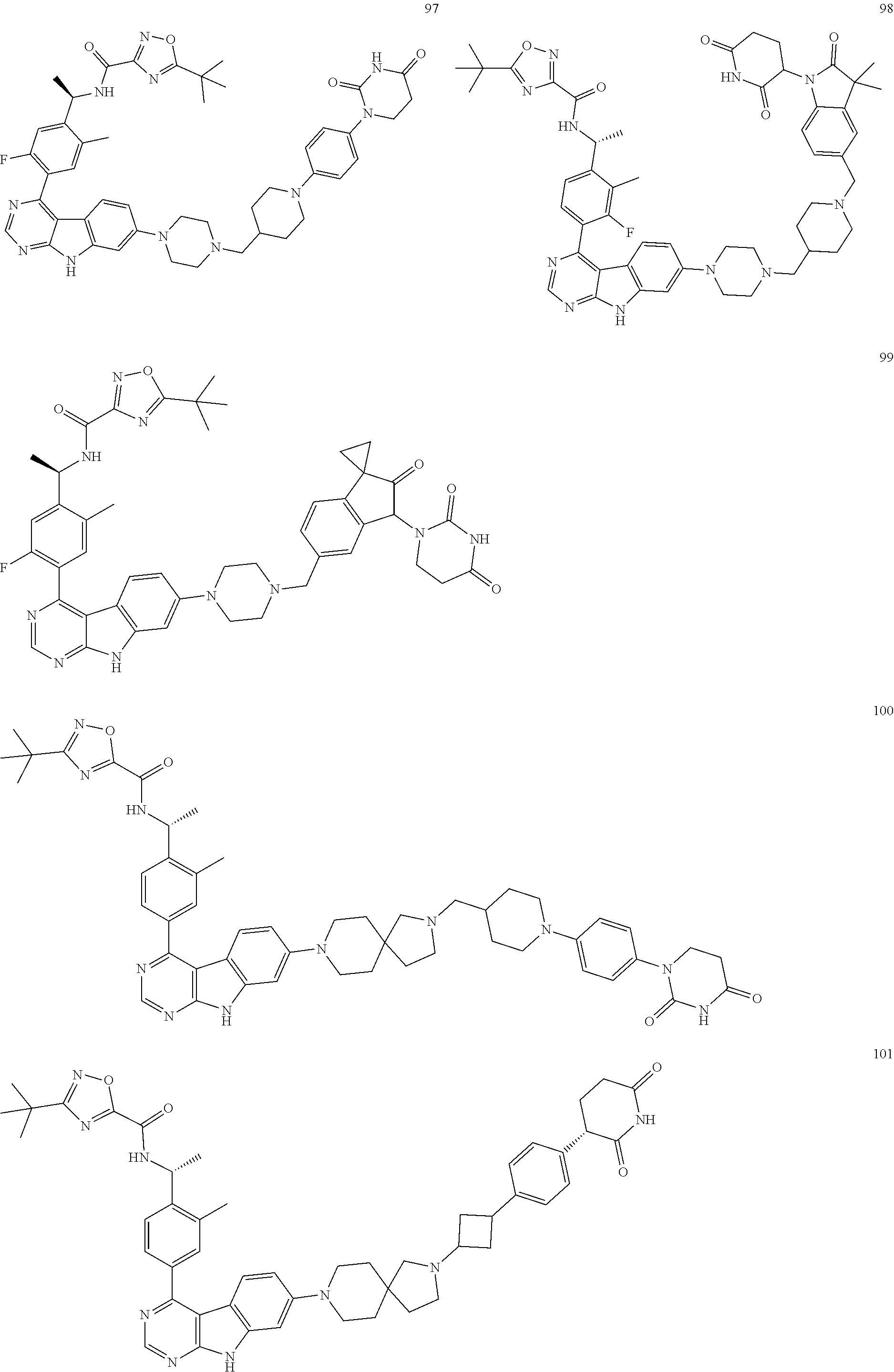

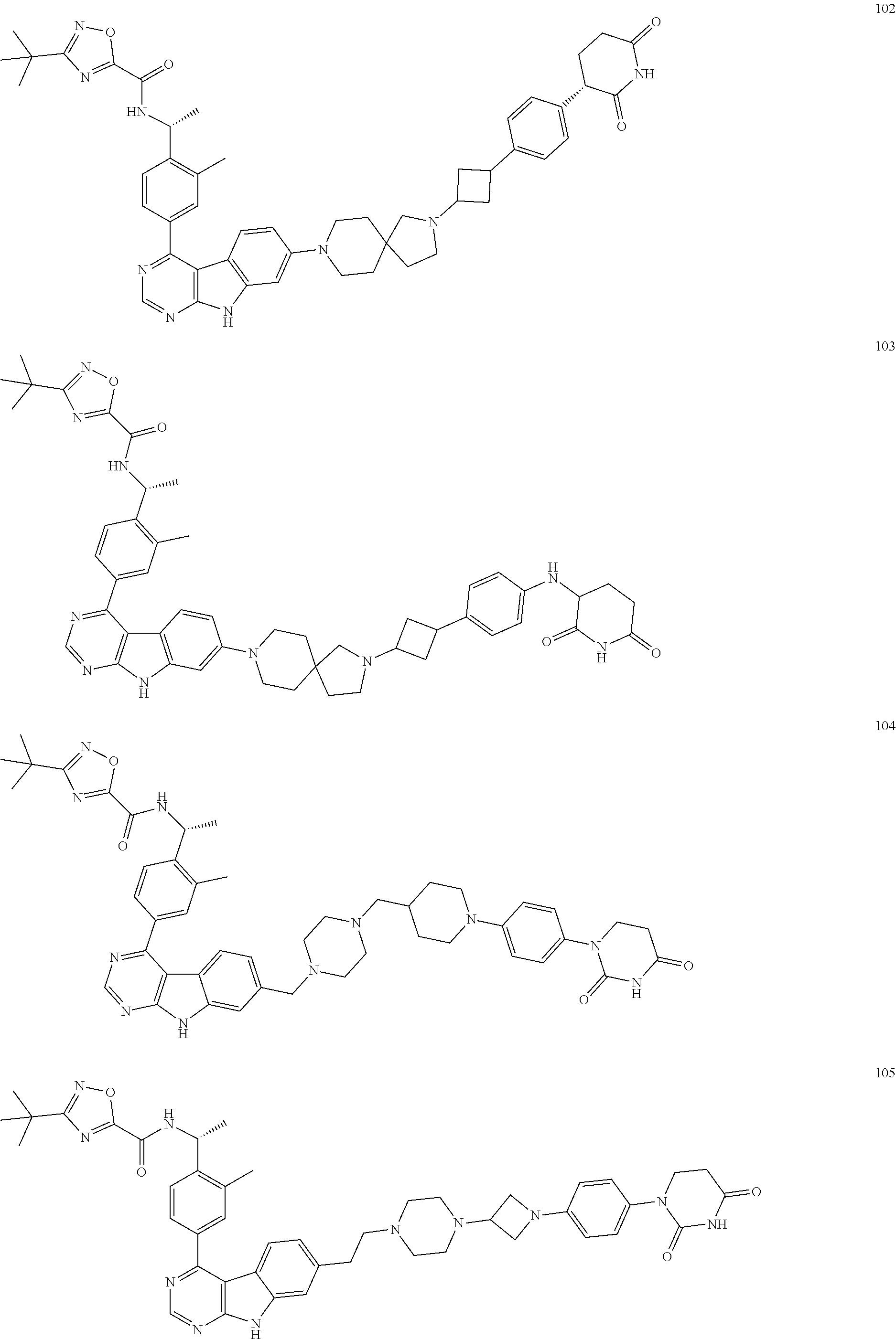 [SMILES: Cc1cc(-c2ncnc3[nH]c4cc(N5CCC6(CC5)CCN(C5CC(c7ccc([C@@H]8CCC(=O)NC8=O)cc7)C5)C6)ccc4c23)ccc1[C@@H](C)NC(=O)c1nc(C(C)(C)C)no1 ; Cc1cc(-c2ncnc3[nH]c4cc(N5CCC6(CC5)CCN(C5CC(c7ccc(NC8CCC(=O)NC8=O)cc7)C5)C6)ccc4c23)ccc1[C@@H](C)NC(=O)c1nc(C(C)(C)C)no1 ; Cc1cc(-c2ncnc3[nH]c4cc(CN5CCN(CC6CCN(c7ccc(N8CCC(=O)NC8=O)cc7)CC6)CC5)ccc4c23)ccc1[C@@H](C)NC(=O)c1nc(C(C)(C)C)no1 ; Cc1cc(-c2ncnc3[nH]c4cc(CCN5CCN(C6CN(c7ccc(N8CCC(=O)NC8=O)cc7)C6)CC5)ccc4c23)ccc1[C@@H](C)NC(=O)c1nc(C(C)(C)C)no1]

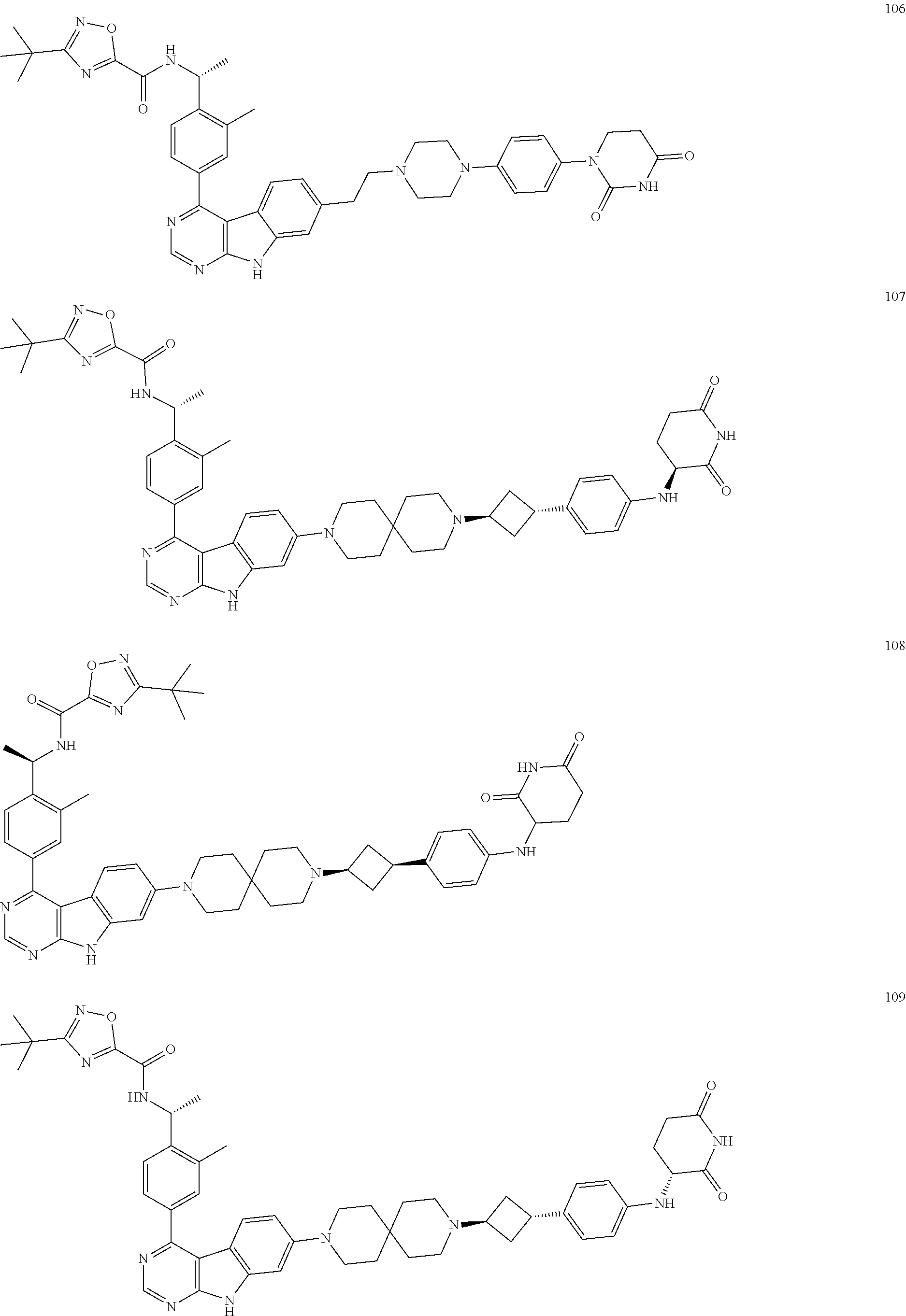

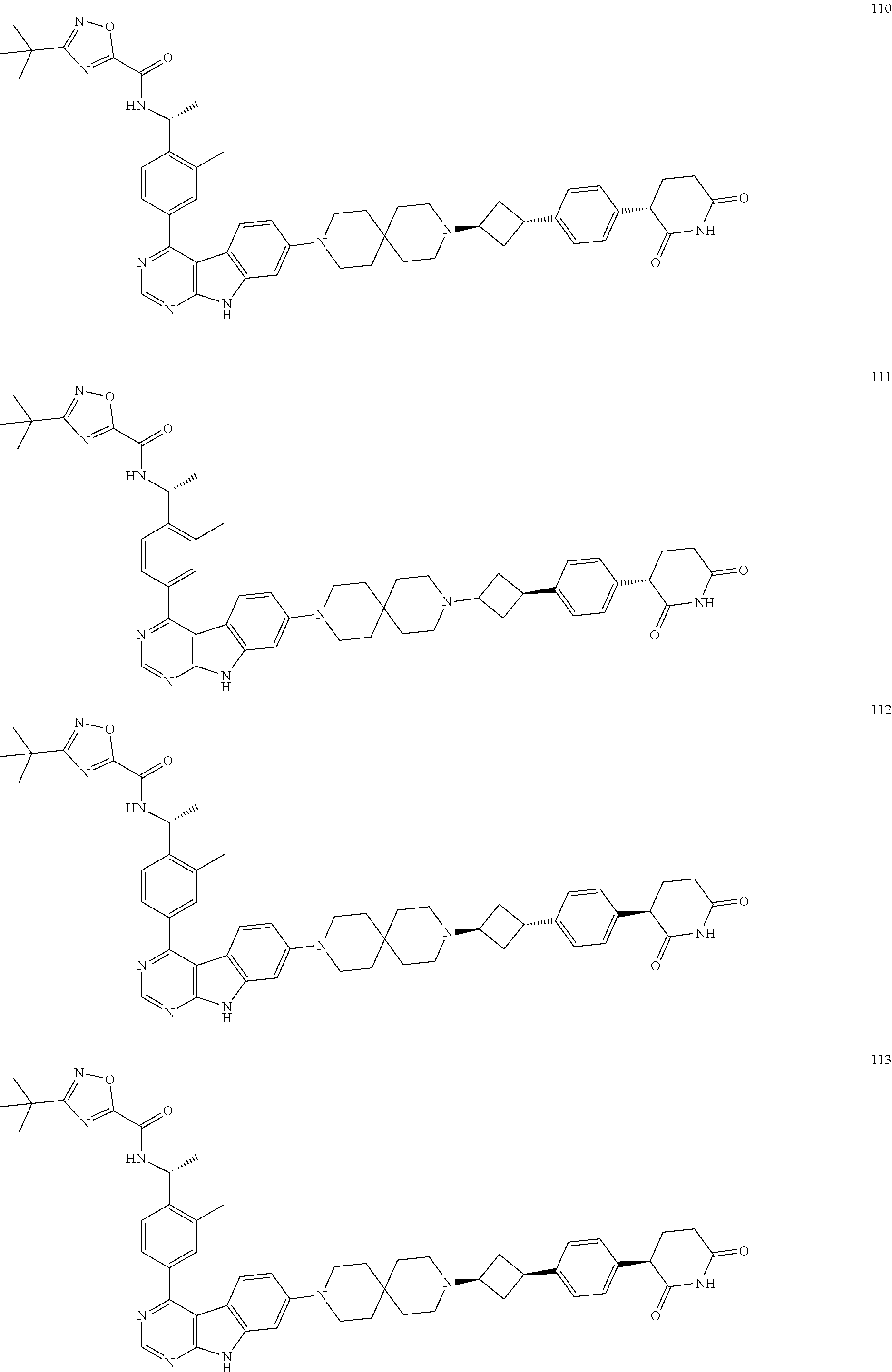

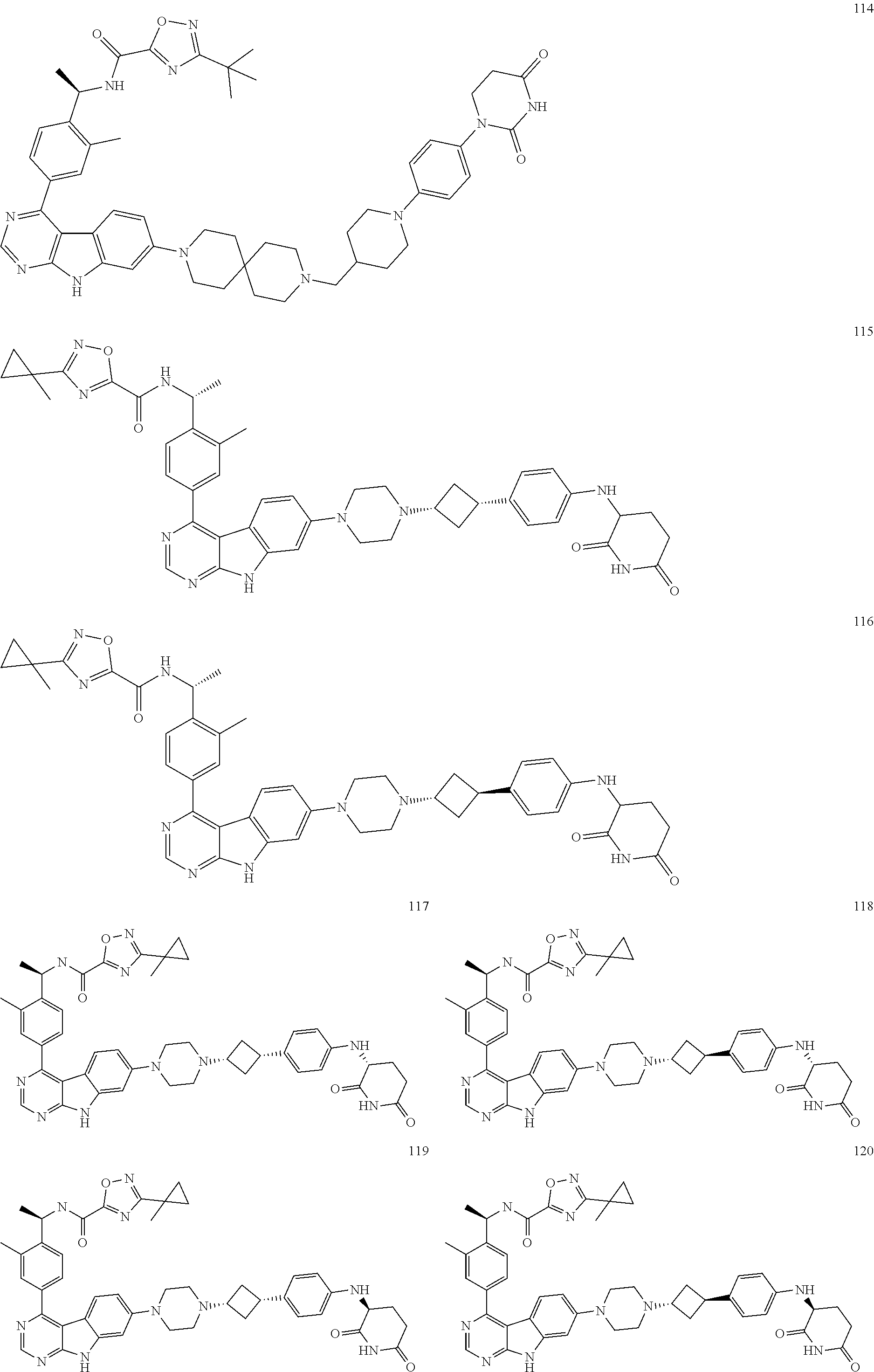

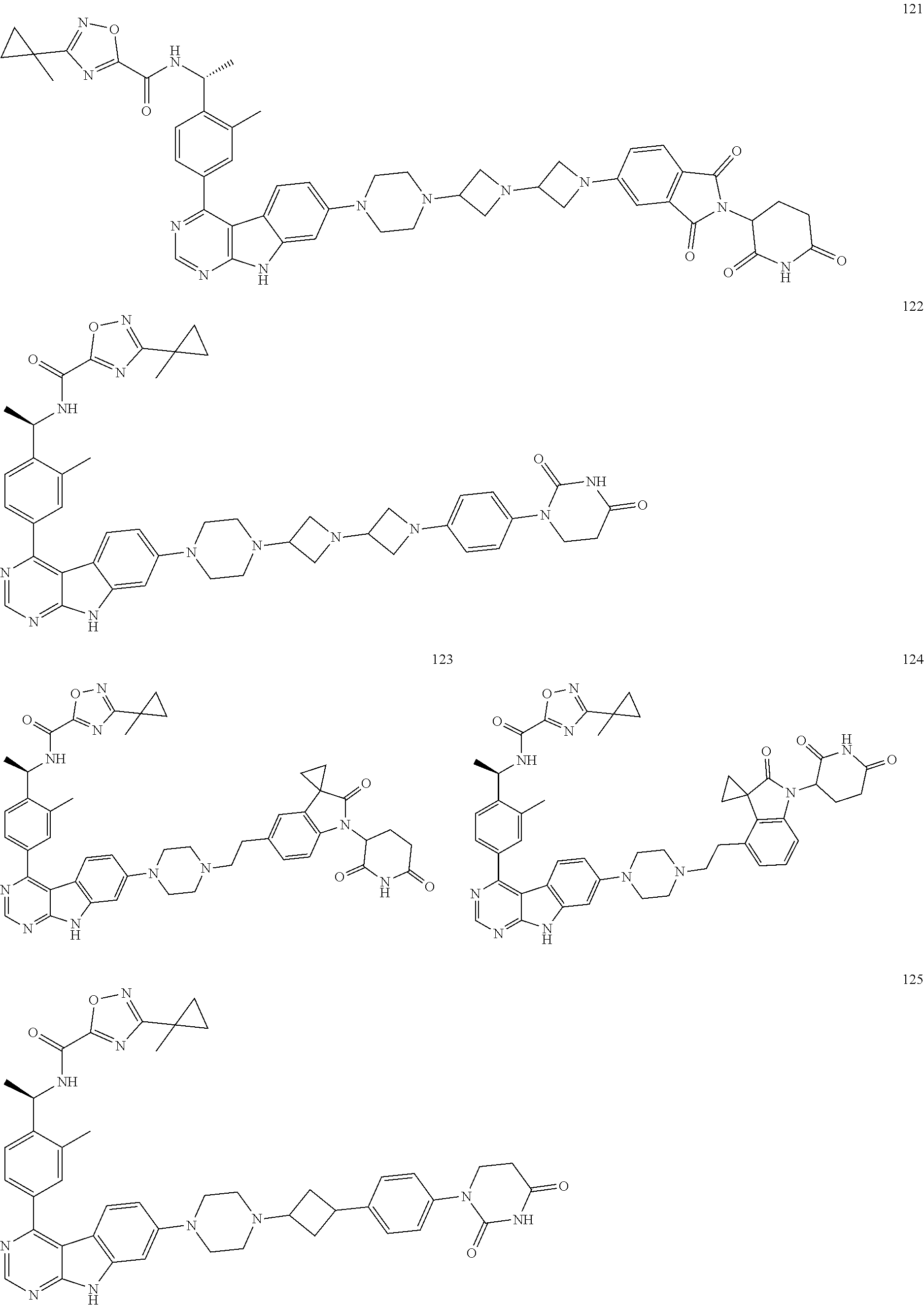

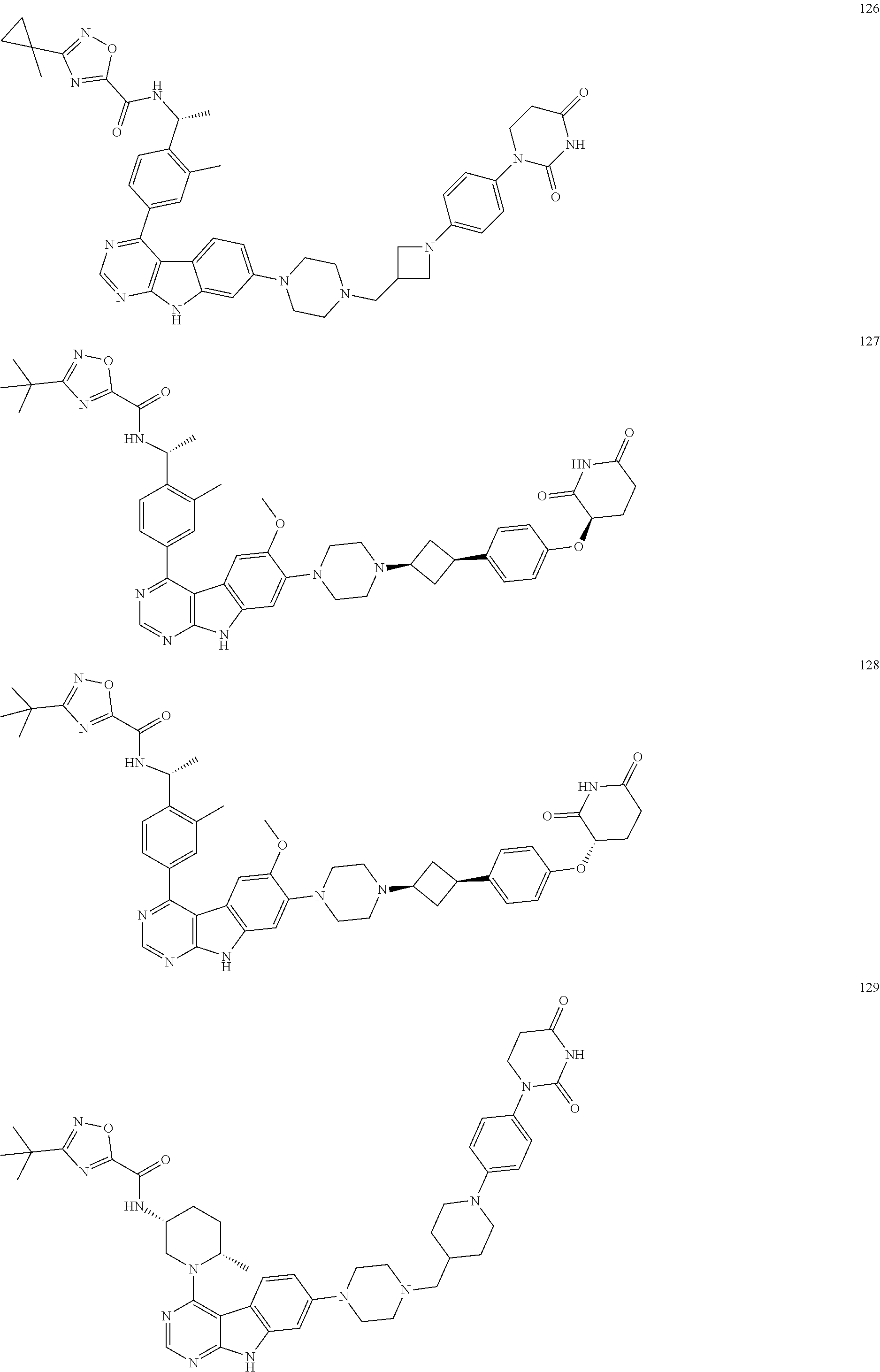 [SMILES: Cc1cc(-c2ncnc3[nH]c4cc(N5CCN(CC6CN(c7ccc(N8CCC(=O)NC8=O)cc7)C6)CC5)ccc4c23)ccc1[C@@H](C)NC(=O)c1nc(C2(C)CC2)no1 ; COc1cc2c(cc1N1CCN([C@H]3C[C@@H](c4ccc(O[C@@H]5CCC(=O)NC5=O)cc4)C3)CC1)[nH]c1ncnc(-c3ccc([C@@H](C)NC(=O)c4nc(C(C)(C)C)no4)c(C)c3)c12 ; COc1cc2c(cc1N1CCN([C@H]3C[C@@H](c4ccc(O[C@H]5CCC(=O)NC5=O)cc4)C3)CC1)[nH]c1ncnc(-c3ccc([C@@H](C)NC(=O)c4nc(C(C)(C)C)no4)c(C)c3)c12 ; C[C@H]1CC[C@@H](NC(=O)c2nc(C(C)(C)C)no2)CN1c1ncnc2[nH]c3cc(N4CCN(CC5CCN(c6ccc(N7CCC(=O)NC7=O)cc6)CC5)CC4)ccc3c12]

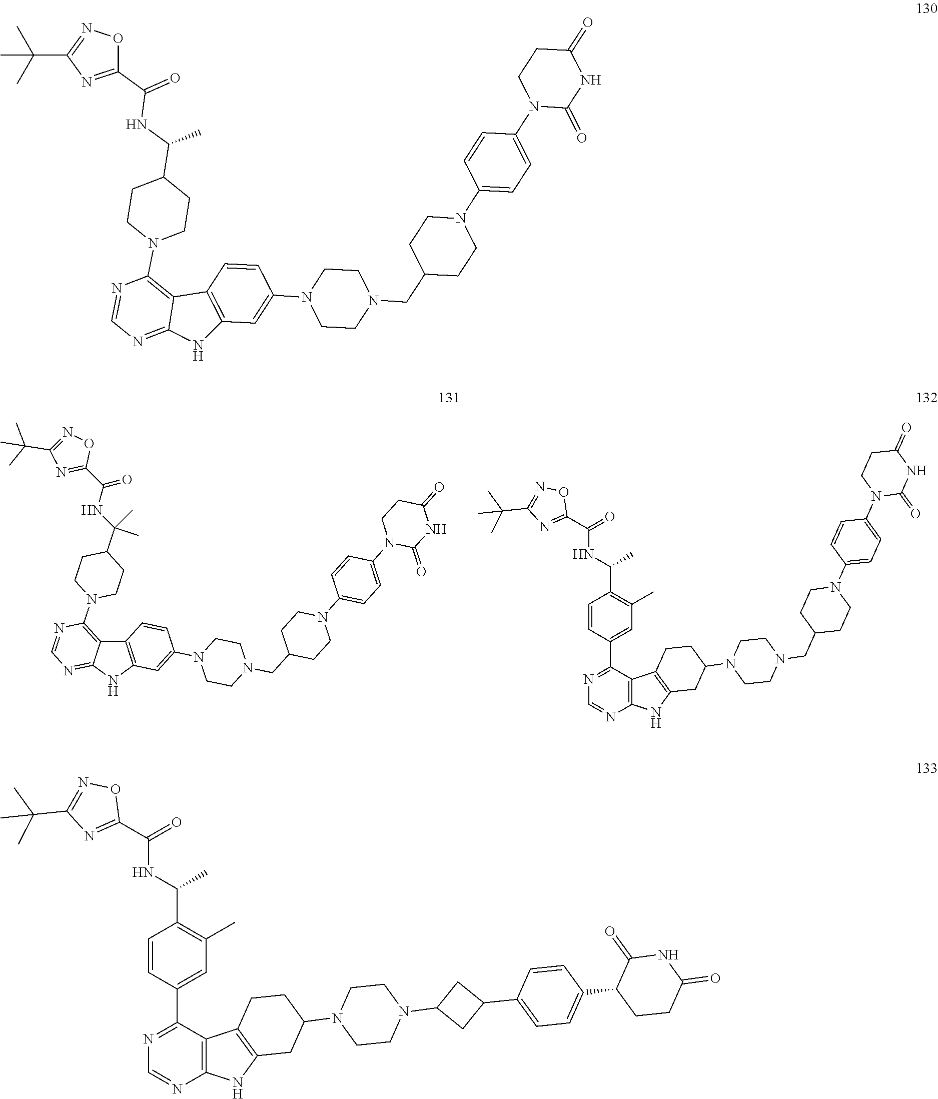 [SMILES: C[C@@H](NC(=O)c1nc(C(C)(C)C)no1)C1CCN(c2ncnc3[nH]c4cc(N5CCN(CC6CCN(c7ccc(N8CCC(=O)NC8=O)cc7)CC6)CC5)ccc4c23)CC1 ; CC(C)(C)c1noc(C(=O)NC(C)(C)C2CCN(c3ncnc4[nH]c5cc(N6CCN(CC7CCN(c8ccc(N9CCC(=O)NC9=O)cc8)CC7)CC6)ccc5c34)CC2)n1 ; Cc1cc(-c2ncnc3[nH]c4c(c23)CCC(N2CCN(CC3CCN(c5ccc(N6CCC(=O)NC6=O)cc5)CC3)CC2)C4)ccc1[C@@H](C)NC(=O)c1nc(C(C)(C)C)no1 ; Cc1cc(-c2ncnc3[nH]c4c(c23)CCC(N2CCN(C3CC(c5ccc([C@H]6CCC(=O)NC6=O)cc5)C3)CC2)C4)ccc1[C@@H](C)NC(=O)c1nc(C(C)(C)C)no1]

Aspect 38. A pharmaceutical composition comprising the compound according to any one of Aspects 1-37, or a pharmaceutically acceptable salt thereof, and at least one pharmaceutically acceptable carrier or excipient.

Aspect 39. A method of inhibiting BTK activity, which comprises administering to an individual the compound according to any one of Aspects 1-37, or a pharmaceutically acceptable salt thereof, including the compound of formula (I) or the specific compounds exemplified herein.

Aspect 40. A method of treating a disease or disorder in a patient comprising administering to the patient a therapeutically effective amount of the compound any one of Aspects 1-37, or a pharmaceutically acceptable salt thereof as an BTK kinase inhibitor, wherein the disease or disorder is associated with inhibition of BTK, preferably, the disease or disorder is cancer.

### DETAILED DESCRIPTION OF THE INVENTION

### Definitions

The following terms have the indicated meaning throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” refers to a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include without limitation to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl ort-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (Cl), bromo (Br) and iodo (I).

The term “haloalkyl” refers to an alkyl group in which one or more hydrogens are replaced by one or more halogen atoms such as fluoro, chloro, bromo, and iodo. Examples of the haloalkyl include without limitation to haloC₁₋₈alkyl, haloC₁₋₆alkyl or halo C₁₋₄alkyl, such as —CF₃, —CH₂Cl, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include without limitation to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include without limitation to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include without limitation to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to cyclopropyl, cyclobutyl, cyclopentyl, and cyclohexyl. Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, preferably cyclohexenyl.

The term “fused cycloalkenyl” refers to a bicyclic cycloalkyl group as defined herein which contain at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond.

The term “fused cycloalkynyl” refers to a bicyclic cycloalkyl group as defined herein which contains at least one triple bond and is formed by two or more rings sharing two adjacent atoms.

The term a “benzo fused cycloalkyl” is a bicyclic fused cycloalkyl in which a 4- to 8-membered monocyclic cycloalkyl ring fused to a benzene ring. For example, a benzo fused cycloalkyl is

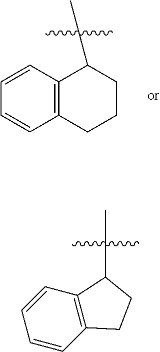 [SMILES: *C1CCCc2ccccc21 ; *C1CCc2ccccc21]

wherein the wavy lines indicate the points of attachment.

The term a “benzo fused cycloalkenyl” is a bicyclic fused cycloalkenyl in which a 4- to 8-membered monocyclic cycloalkenyl ring fused to a benzene ring.

The term a “benzo fused cycloalkynyl” is a bicyclic fused cycloalkynyl in which a 4- to 8-membered monocyclic cycloalkynyl ring fused to a benzene ring.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2, 3-dihydro-1H-indenyl, 1H-indenyl, 1, 2, 3, 4-tetralyl, 1, 4-dihydronaphthyl, etc. Preferred embodiments are 8 to 9 membered fused rings, which refer to cyclic structures containing 8 to 9 ring atoms within the above examples.

The term “aryl” used alone or in combination with other terms refers to a group selected from:
a) 5- and 6-membered carbocyclic aromatic rings, e.g., phenyl;
b) bicyclic ring systems such as 7 to 12 membered bicyclic ring systems, wherein at least one ring is carbocyclic and aromatic, e.g., naphthyl and indanyl; and,
c) tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include without limitation to phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene.

The term “heteroaryl” refers to a group selected from:
a) 5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
b) 7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
c) 11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

Representative examples of bicyclic fused heteroaryl include without limitation to the following groups: benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Aromatic ring” refers to aromatic carbocycles (e.g., the above-mentioned aryl) and aromatic heterocycles (e.g., the above-mentioned heteroaryl).

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur” used herein refer to S, SO or SO₂.

The term “monocyclic heterocyclyl” refers to monocyclic groups in which at least one ring member (e.g., 1-3 heteroatoms, 1 or 2 heteroatom(s)) is a heteroatom selected from nitrogen, oxygen or optionally oxidized sulfur. A heterocycle may be saturated or partially saturated.

Exemplary monocyclic 4 to 9-membered heterocyclyl groups include without limitation to pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1, 2-dithietanyl, 1, 3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1, 4-oxathianyl, 1, 4-dioxepanyl, 1, 4-oxathiepanyl, 1, 4-oxaazepanyl, 1, 4-dithiepanyl, 1, 4-thiazepanyl and 1, 4-diazepanyl, 1, 4-dithianyl, 1, 4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl could be mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered, 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include without limitation to the following groups: 2, 3-dihydrospiro[indene-1, 2′-pyrrolidine] (e.g., 2, 3-dihydrospiro[indene-1, 2′-pyrrolidine]-1′-yl), 1, 3-dihydrospiro[indene-2, 2′-pyrrolidine] (e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3]heptane (e.g., 2-oxa-6-azaspiro[3.3]heptan-6-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1, 7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl could be bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include without limitation to the following groups: octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, ortetrahydropyrazolopyrimidinyl (e.g., 4, 5, 6, 7-tetrahydropyrazolo[1, 5-a]pyrimidin-3-yl).

The term a “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl could be bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include without limitation to the following groups: 2-azabicyclo[2.2.1]heptyl, azabicyclo[3.1.0]hexyl, 2-azabicyclo[2.2.2]octyl and 2-azabicyclo[3.3.2]decyl.

The term “at least one substituent” disclosed herein includes, for example, from 1 to 4, such as from 1 to 3, further as 1 or 2, substituents, provided that the theory of valence is met. For example, “at least one substituent R^{6d}” disclosed herein includes from 1 to 4, such as from 1 to 3, further as 1 or 2, substituents selected from the list of R^{6d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical or chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers and diastereomers can also be separated by the use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller C. H., et al. “Chromatographic resolution of enantiomers: Selective review. “J. Chromatogr, 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W, Ed. Drug Stereochemistry: Analytical Methods and Pharmacology. New York: Marcel Dekker, Inc., 1993.

“Pharmaceutically acceptable salts” refer to those salts which are, within the scope of sound medical judgment, suitable for use in contact with the tissues of humans and animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic agent, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical route to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc., a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, an aromatic, a sweetener, a dye and etc.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₄, C₁₋₆, and the like.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz.

¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time | | |
|---|---|---|
| (min) | A(%) | B(%) |
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time | | |
|---|---|---|
| (min) | A(%) | B(%) |
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time | | |
|---|---|---|
| (min) | A(%) | B(%) |
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on any commercially available column (e.g., 150×21.2 mm ID, 5 pm, Gemini NXC 18, Waters Xselect CSH C18, or Waters Xbridge C18) at a flow rate of 20 ml/min, injection volume of 2 ml, at room temperature and UV Detection at 214 nm and 254 nm. Mobile phase A is ACN (optionally with 0.1% FA); Mobile phase B is water (optionally with 0.1% FA or 0.03% NH₃H₂O). Gradient Table: Mobile Phase A (20%-90%, 30%-90%, 40%-90% or 50%-90%), Time (min): 0-15 min, 0-17 min, or 0-20 min.

In the following examples, the abbreviations below are used:

| | |
|---|---|
| AcOH | Acetic acid |
| AcOK | Potassium acetate |
| Aq | Aqueous |
| ACN | Acetonitrile |
| Brine | Saturated aqueous sodium chloride solution |
| Bn | Benzyl |
| BnBr | Benzyl Bromide |
| Boc | Tert-butyloxycarbonyl |
| (Bpin)₂ | 4,4,4′,4′,5,5,5′,5′-octamethyl- |
| | 2,2′-bi(1,3,2-dioxaborolane) |
| BPO | Dibenzoyl peroxide |
| BINAP | (±)-2,2′-Bis(diphenylphosphino)-1,1′-binaphthalene |
| Cbz | benzyloxycarbonyl |
| CH₂Cl₂ | Dichloromethane |
| CoCl₂ | Cobalt chloride |
| DCE | dichloroethane |
| DMF | N,N-Dimethylformamide |
| Dppf | 1,1″-bis(diphenylphosphino)ferrocene |
| DBU | 1,8-diazabicyclo[5.4.0]undec-7-ene |
| DCM | Dichloromethane |
| DIAD | Diisopropyl azodicarboxylate |
| DIEA or | N,N-diisopropylethylamine |
| DIPEA | |
| DIBAL-H | Diisobutylaluminium hydride |
| DMAP | 4-N,N-dimethylaminopyridine |
| DMF | N,N-dimethylformamide |
| DMSO | Dimethyl sulfoxide |
| EA or EtOAc | Ethyl acetate |
| EtOH | Ethanol |
| Et₂O or ether | Diethyl ether |
| FA | Formic acid |
| g | Grams |
| h or hr | Hour |
| HATU | O-(7-Azabenzotriazol-1-yl)- |
| | N,N,N′,N′-tetramethyluronium |
| | hexafluorophosphate |
| HBTU | 2-(1H-Benzotriazole-1-yl)-1,1,3,3-tetramethylaminium |
| | hexafluorophosphate |
| HCl | Hydrochloric acid |
| Hex | Hexane |
| HPLC | High-performance liquid chromatography |
| IBX | 2-iodoxybenzoic acid |
| IPA | 2-propanol |
| i-PrOH | Isopropyl alcohol |
| LiHMDS | lithium bis(trimethylsilyl)azanide |
| mg | Milligrams |
| mL | Milliliters |
| Mmol | Millimole |
| MeCN | Acetonitrile |
| MeOH | Methanol |
| Min | Minutes |
| ms or MS | Mass spectrum |
| MsCl | methanesulfonyl chloride |
| MsOH | Methanesulfonic acid |
| Na₂SO₄ | Sodium sulfate |
| NBS | N-Bromosuccinimide |
| NMP | N-Methyl pyrrolidone |
| PE | Petroleum ether |
| Ph₃PO | Triphenyl phosphorus oxide |
| PhMe | Toluene |
| PPh₃ | triphenylphosphine |
| PPA | Polyphosphoric acid |
| PIN | pinacol |
| Rt | Retention time |
| R.T. or r.t. | Room temperature |
| Ruphos | 2-Dicyclohexylphosphino-2′,6′-diisopropoxybiphenyl |
| SM | Starting material |
| STAB | Sodium triacetoxyborohydride |
| T₃P | Propylphosphonic anhydride |
| TBAF | Tetra-butyl ammonium fluoride |
| TBSCl | tert-Butyldimethylsilyl chloride |
| TEA | Triethanolamine |
| TFA | Trifluoroacetic acid |
| Tf₂O | Triflic anhydride |
| THF | Tetrahydrofuran |
| TLC | thin layer chromatography |
| TMSOK | trimethyl(potassiooxy)silane |
| Ts | para-toluenesulfonyl |
| Ts Cl | p-Toluenesulfonyl chloride |
| TsOH | 4-toluene sulfonic acid |
| TBS | tert-butyldimethylsilyl |
| μL | Microliters |
| Brettphos | Dicyclohexyl[3,6-dimethoxy-2′,4′,6′- |
| | tris(1-methylethyl)[1,1′-biphenyl]-2-yl]phosphine |
| Pd₂(dba)₂ | Di(dibenzylideneacetone)dipalladium |
| Prep-HPLC | preparative high performance liquid chromatography |
| Prep-TLC | preparative thin layer chromatography |

### Example 1: 3-(tert-butyl)-N-(4-(6-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2-methylbenzyl)-1,2,4-oxadiazole-5-carboxamide

### Step 1: tert-butyl 4-(3-chloro-4-nitrophenyl)piperazine-1-carboxylate

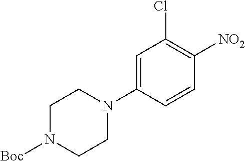 [SMILES: CC(C)(C)OC(=O)N1CCN(c2ccc([N+](=O)[O-])c(Cl)c2)CC1]

A mixture of 2-chloro-4-fluoro-1-nitrobenzene (25 g, 142.4 mmol), tert-butyl piperazine-1-carboxylate (29 g, 156.7 mmol) and K₂CO₃ (59 g, 427.2 mmol) in DMF (250 mL) was stirred at 100° C. for 16 h. The mixture was cooled and water (250 mL) was added. The precipitated solids were collected by filtration and washed with water (2×100 mL). The resulting solid was dried to afford the product (46 g, 95%).

### Step 2: tert-butyl 4-(3-(1-cyano-2-ethoxy-2-oxoethyl)-4-nitrophenyl)piperazine-1-carboxylate

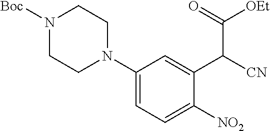 [SMILES: CCOC(=O)C(C#N)c1cc(N2CCN(C(=O)OC(C)(C)C)CC2)ccc1[N+](=O)[O-]]

To a stirring solution of NaH (60% in mineral oil) (6.4 g, 160.9 mmol, 60%) in DMF (250 mL) was added ethyl cyanoacetate (12.4 g, 109.7 mmol) dropwise at 0° C. under N₂ atmosphere. The mixture was stirred for 1 h at room temperature. To the above solution was added tert-butyl 4-(3-chloro-4-nitrophenyl)piperazine-1-carboxylate (25 g, 73.144 mmol). The resulting mixture was stirred for 16 h at 70° C. under N₂ atmosphere. The mixture was cooled and water (200 mL) was added. The resulting mixture was acidified to pH=6 with 1M HCl (aq.) and extracted with DCM (3×250 mL). The combined organic layers were washed with sat. NaCl (aq.) (200 mL) and dried over anhydrous Na₂SO₄. After filtration, the filtrate was concentrated under reduced pressure. The residue was purified by silica gel column chromatography, eluting with hexane/EtOAc (1:1) to afford the product (22 g, 72%). [M+H]⁺=419.2.

### Step 3: ethyl 2-amino-5-(4-(tert-butoxycarbonyl)piperazin-1-yl)-1H-indole-3-carboxylate

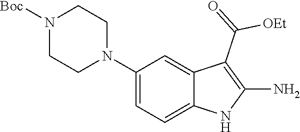 [SMILES: CCOC(=O)c1c(N)[nH]c2ccc(N3CCN(C(=O)OC(C)(C)C)CC3)cc12]

To a solution of tert-butyl 4-(3-(1-cyano-2-ethoxy-2-oxoethyl)-4-nitrophenyl)piperazine-1-carboxylate (10 g, 23.9 mmol) in AcOH (100 mL) was added Zn (7.8 g, 119.5 mmol). The resulting mixture was stirred at room temperature for 3 h. The mixture was filtered and the filter cake was washed with AcOH (2×20 mL). The filtrate was concentrated under reduced pressure. The residue was purified by silica gel column chromatography, eluting with hexane/EtOAc(1:1) to afford the product (7.8 g, 84%). [M+H]⁺=389.1.

### Step 4: tert-butyl 4-(4-oxo-4,9-dihydro-3H-pyrimido[4,5-b]indol-6-yl)piperazine-1-carboxylate

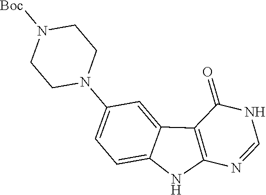 [SMILES: CC(C)(C)OC(=O)N1CCN(c2ccc3[nH]c4nc[nH]c(=O)c4c3c2)CC1]

A solution of ethyl 2-amino-5-(4-(tert-butoxycarbonyl)piperazin-1-yl)-1H-indole-3-carboxylate (6 g, 15.4 mmol) and formamidine (3.2 g, 30.9 mmol) in methyl glycol (60 mL) was stirred for 24 hours at 120° C. The resulting mixture was cooled and concentrated under reduced pressure. The residue was basified to pH=8 with sat. NaHCO₃ (aq.). The resulting mixture was extracted with DCM (3×100 mL). The combined organic layers were washed with sat. NaCl (aq.) (50 mL) and dried over anhydrous Na₂SO₄. After filtration, the filtrate was concentrated under reduced pressure. The residue was purified by silica gel column chromatography, eluted with a gradient of DCM:MeOH 1:0 to 10:1 to afford the product (5 g, 88%). [M+H]⁺=370.2.

### Step 5: 4-chloro-6-(piperazin-1-yl)-9H-pyrimido[4,5-b]indole

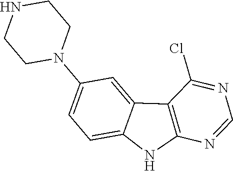 [SMILES: Clc1ncnc2[nH]c3ccc(N4CCNCC4)cc3c12]

A mixture of tert-butyl 4-(4-oxo-4,9-dihydro-3H-pyrimido[4,5-b]indol-6-yl)piperazine-1-carboxylate (5 g, 13.5 mmol) and POCl₃ (10 mL) in dioxane (10 mL) was stirred for 4 hours at 90° C. The mixture was cooled. Water (15 mL) and EA (20 mL) were added, then the mixture was basified to pH=8 with sat. NaHCO₃ (aq.). The resulting mixture was filtered, the filter cake was washed with water (2×10 mL). The filtrate was concentrated under reduced pressure to afford the product (2.4 g, 62%). [M+H]⁺=288.2.

### Step 6: tert-butyl 4-(4-chloro-9H-pyrimido[4,5-b]indol-6-yl)piperazine-1-carboxylate

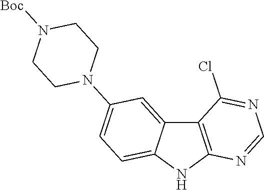 [SMILES: CC(C)(C)OC(=O)N1CCN(c2ccc3[nH]c4ncnc(Cl)c4c3c2)CC1]

To a stirred solution of 4-chloro-6-(piperazin-1-yl)-9H-pyrimido[4,5-b]indole (500 mg, 1.3 mmol) and NaHCO₃ (218 mg, 2.6 mmol) in THF (10 mL) and H₂O (5 mL) was added Boc₂O (305 mg, 1.4 mmol) in portions at 0° C. The resulting mixture was stirred for 16 h at room temperature. The resulting mixture was concentrated under reduced pressure. To the resulting mixture was added water (20 mL) and extracted with EtOAc (3×50 mL). The combined organic layers were washed with sat. NaCl (aq.) (20 mL) and dried over anhydrous Na₂SO₄. After filtration, the filtrate was concentrated under reduced pressure. The residue was purified by silica gel column chromatography, eluted with EA to afford the product (150 mg, 29.8%). [M+H]⁺=388.2.

### Step 7: tert-butyl 4-(4-(4-((3-(tert-butyl)-1,2,4-oxadiazole-5-carboxamido)methyl)-3-methylphenyl)-9H-pyrimido[4,5-b]indol-6-yl)piperazine-1-carboxylate

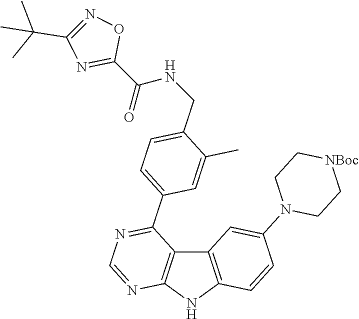 [SMILES: Cc1cc(-c2ncnc3[nH]c4ccc(N5CCN(C(=O)OC(C)(C)C)CC5)cc4c23)ccc1CNC(=O)c1nc(C(C)(C)C)no1]

A mixture of tert-butyl 4-(4-chloro-9H-pyrimido[4,5-b]indol-6-yl)piperazine-1-carboxylate (120 mg, 0.3 mmol), 3-(tert-butyl)-N-(2-methyl-4-(4,4,5,5-tetramethyl-1,3,2-dioxaborolan-2-yl)benzyl)-1,2,4-oxadiazole-5-carboxamide (the compound was obtained through the similar way in US20160096834A1) (161 mg, 0.4 mmol), Pd(dppf)Cl₂ (23 mg, 0.031 mmol) and K₃PO₄ (131 mg, 0.6 mmol) in dioxane (3 mL) and water (0.5 mL) was stirred for 4 hours at 80° C. under N₂ atmosphere. The mixture was cooled. To the resulting mixture was added water (15 mL) and extracted with DCM (3×20 mL). The combined organic layers were washed with sat. NaCl (15 mL) and dried over anhydrous Na₂SO₄. After filtration, the filtrate was concentrated under reduced pressure. The resulting residue was purified by silica gel column chromatography, eluting with DCM/MeOH(10:1) to afford the product (130 mg, 67%). [M+H]⁺=625.0.

### Step 8: 3-(tert-butyl)-N-(2-methyl-4-(6-(piperazin-1-yl)-9H-pyrimido[4,5-b]indol-4-yl)benzyl)-1,2,4-oxadiazole-5-carboxamide

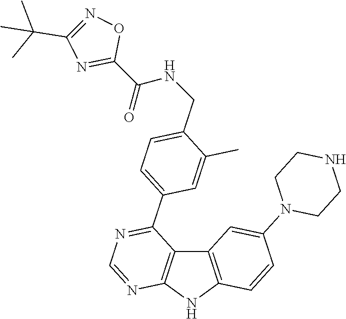 [SMILES: Cc1cc(-c2ncnc3[nH]c4ccc(N5CCNCC5)cc4c23)ccc1CNC(=O)c1nc(C(C)(C)C)no1]

To a stirred solution of tert-butyl 4-(4-(4-((3-(tert-butyl)-1,2,4-oxadiazole-5-carboxamido)methyl)-3-methylphenyl)-9H-pyrimido[4,5-b]indol-6-yl)piperazine-1-carboxylate (130 mg, 0.2 mmol) in DCM (1 mL) was added TFA (1 mL) at room temperature. The resulting solution was stirred for 2 h at room temperature. The resulting mixture was concentrated under vacuum. The residue was purified by reverse phase flash chromatography with the following conditions: column, C18; mobile phase A, 0.1% FA in water, mobile phase B, ACN, 10% to 50% (B %) gradient in 25 min; detector, UV 254 nm to afford the product (53 mg, 45%). ¹H NMR (300 MHz, DMSO) δ 12.26 (s, 1H), 9.96-9.92 (m, 1H), 8.91 (s, 1H), 8.23 (s, 1H), 7.79-7.74 (m, 2H), 7.56-7.47 (m, 2H), 7.34-7.28 (m, 2H), 4.60 (d, J=5.7 Hz 2H), 2.98-3.02 (m, 8H), 2.51-2.49 (m, 3H), 1.43-1.34 (m, 9H); [M+H]⁺=525.2.

### Step 9: 3-(tert-butyl)-N-(4-(6-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2-methylbenzyl)-1,2,4-oxadiazole-5-carboxamide

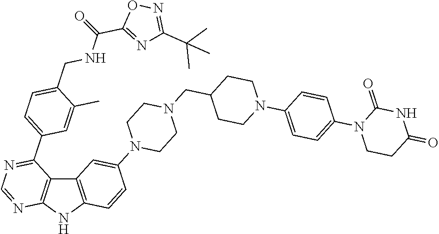 [SMILES: Cc1cc(-c2ncnc3[nH]c4ccc(N5CCN(CC6CCN(c7ccc(N8CCC(=O)NC8=O)cc7)CC6)CC5)cc4c23)ccc1CNC(=O)c1nc(C(C)(C)C)no1]

3-(tert-Butyl)-N-(2-methyl-4-(6-(piperazin-1-yl)-9H-pyrimido[4,5-b]indol-4-yl)benzyl)-1,2,4-oxadiazole-5-carboxamide (50 mg, 0.1 mmol) was dissolved in MeOH/DCM(10:1, 2 mL). 1-(4-(2,4-Dioxotetrahydropyrimidin-1(2H)-yl)phenyl)piperidine-4-carbaldehyde (30 mg, 0.1 mmol) was added to the solution in portions and the resulting mixture was stirred at room temperature for 16 h until LC-MS indicated all the starting material was consumed. The mixture was concentrated and purified with SiO₂-gel column (DCM:MeOH=10:1) to give the titled product (29 mg, 35.8%). ¹H NMR (400 MHz, DMSO) δ 12.21 (s, 1H), 10.26 (s, 1H), 9.95 (s, 1H), 8.90 (s, 1H), 7.77-7.73 (m, 2H), 7.56-7.53 (m, 1H), 7.51-7.43 (m, 1H), 7.34 (s, 1H), 7.30-7.24 (m, 1H), 7.14-7.12 (m, 2H), 7.03-6.90 (m, 2H), 4.59 (s, 2H), 3.69-3.67 (m, 4H), 3.34-3.32 (m, 4H), 3.03-2.97 (m, 4H), 2.48-2.46 (m, 4H), 2.25-2.22 (m, 4H), 2.20-2.19 (m, 2H), 1.85-1.74 (m, 3H), 1.36 (s, 9H), 1.24-1.23 (m, 1H); [M+H]⁺=810.4.

### Example 2: 3-(tert-butyl)-N-(4-(6-(1-((1-(4-(2,4-dioxotetrahydropyrimidin-1(2H)-yl)phenyl)piperidin-4-yl)methyl)piperidin-4-yl)-9H-pyrimido[4,5-b]indol-4-yl)-2-methylbenzyl)-1,2,4-oxadiazole-5-carboxamide

### Step 1: ethyl 2-(5-bromo-2-nitrophenyl)-2-cyanoacetate

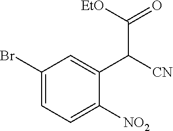 [SMILES: CCOC(=O)C(C#N)c1cc(Br)ccc1[N+](=O)[O-]]

To a stirred solution of NaH (60% in mineral oil, 10.2 g, 253.8 mmol) in DMF (300 mL) was added ethyl cyanoacetate (21.5 g, 190.3 mmol) dropwise at 0° C. under N₂ atmosphere. The mixture was stirred for 1 h at room temperature. To the above solution was added 4-bromo-2-chloro-1-nitrobenzene (44.7 g, 190.3 mmol). The resulting mixture was stirred for 5 h at 70° C. under N₂ atmosphere. The mixture was cooled and water (100 mL) was added. The resulting mixture was acidified to pH=6 with 1 M HCl (aq.) and extracted with DCM (3×250 mL). The combined organic layers were washed with sat. NaCl (aq.) (200 mL) and dried over anhydrous Na₂SO₄. After filtration, the filtrate was concentrated under reduced pressure. The residue was purified by silica gel column chromatography, eluting with a gradient of hexane/EA=10:1 to afford the product (34 g, 85.6%). [M−H]⁻=311.0.

### Step 2: ethyl 2-amino-5-bromo-1H-indole-3-carboxylate

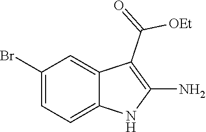 [SMILES: CCOC(=O)c1c(N)[nH]c2ccc(Br)cc12]

To a solution of ethyl 2-(5-bromo-2-nitrophenyl)-2-cyanoacetate (2.00 g, 76.7 mmol) in AcOH (100 mL) was added Zn (3.9 g, 59.7 mmol) in portions at rt for 1 h under N₂ atmosphere. The resulting mixture was stirred at 70° C. for 24 h. The mixture was filtered and the filter cake was washed with AcOH (2×10 mL). The filtrate was concentrated under reduced pressure. To the residue was added water (200 mL). The mixture was basified to pH=8 with sat. NaHCO₃ (aq.) and extracted with EA (3×200 mL). The residue was purified by silica gel column chromatography, eluting with a gradient of hexane:EtOAc 1:1 to afford the product (7.5 g, 34.6%). [M+H]⁺=285.0.

### Step 3: 6-bromo-3,9-dihydro-4H-pyrimido[4,5-b]indol-4-one

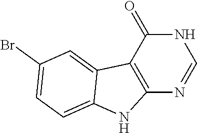 [SMILES: O=c1[nH]cnc2[nH]c3ccc(Br)cc3c12]

A solution of ethyl 2-amino-5-bromo-1H-indole-3-carboxylate (7.5 g, 26.5 mmol) and formamidine (13.8 g, 132.5 mmol) in methyl glycol (75 mL) was stirred for 24 h at 120° C. The resulting mixture was cooled, concentrated and water (100 mL) was added. The precipitated solids were collected by filtration and washed with water (2×20 mL). The resulting solid was dried to afford the product (6.5 g, 92.9%). [M+H]⁺=266.0.

### Step 4: tert-butyl 4-(4-oxo-4,9-dihydro-3H-pyrimido[4,5-b]indol-6-yl)-3,6-dihydropyridine-1(2H)-carboxylate

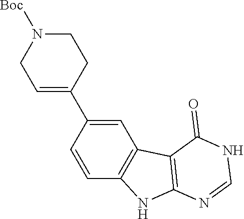 [SMILES: CC(C)(C)OC(=O)N1CC=C(c2ccc3[nH]c4nc[nH]c(=O)c4c3c2)CC1]

A mixture of 6-bromo-3,9-dihydro-4H-pyrimido[4,5-b]indol-4-one (500 mg, 1.893 mmol), tert-butyl 4-(4,4,5,5-tetramethyl-1,3,2-dioxaborolan-2-yl)-3,6-dihydro-2H-pyridine-1-carboxylate (761 mg, 2.461 mmol), Pd(dppf)Cl₂(138 mg, 0.189 mmol) and K₃PO₄(804 mg, 3.787 mmol) in dioxane (5 mL) and H₂O (1 mL) was stirred for 3 h at 90° C. under N₂ atmosphere. The resulting mixture was concentrated under reduced pressure. The residue was purified by silica gel column chromatography, eluted with DCM:MeOH (100:1 to 10:1) to afford the product (366 mg, 52.85%). [M+H]⁺=367.1.

### Step 5: tert-butyl 4-(4-oxo-4,9-dihydro-3H-pyrimido[4,5-b]indol-6-yl)piperidine-1-carboxylate

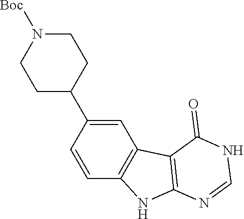 [SMILES: CC(C)(C)OC(=O)N1CCC(c2ccc3[nH]c4nc[nH]c(=O)c4c3c2)CC1]

To a stirred mixture of tert-butyl 4-(4-oxo-4,9-dihydro-3H-pyrimido[4,5-b]indol-6-yl)-3,6-dihydropyridine-1(2H)-carboxylate (1.5 g, 4.1 mmol) in MeOH (15 mL) was added Pd/C (1.7 g, 16.4 mmol). The resulting mixture was stirred for 16 h at rt under H₂ atmosphere. The resulting mixture was filtered and the filter cake was washed with MeOH (2×15 mL). The filtrate was concentrated under reduced pressure. The residue was purified by reverse phase flash chromatography with the following conditions: column, C18; mobile phase A, 10 mM NH₄HCO₃ in water, mobile phase B: ACN; 20% to 60% (B %) gradient in 25 min; detector, UV 254 nm to afford the product (410 mg, 27.18%). [M+H]⁺=369.2.

### Step 6: 4-chloro-6-(piperidin-4-yl)-9H-pyrimido[4,5-b]indole

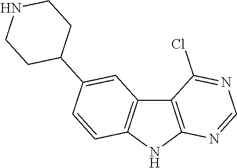 [SMILES: Clc1ncnc2[nH]c3ccc(C4CCNCC4)cc3c12]

To a stirred solution of tert-butyl 4-(4-oxo-4,9-dihydro-3H-pyrimido[4,5-b]indol-6-yl)piperidine-1-carboxylate (200 mg, 2.3 mmol) in dioxane (10 mL) was added POCl₃ (10 mL, 107.3 mmol) dropwise at ambient temperature. The resulting mixture was stirred for 3 h at 100° C. and concentrated. The residue was diluted with toluene (100 mL) and concentrated under reduced pressure. Repeat this work 3 times to afford the product (410 mg, crude). [M+H]⁺=287.1.

### Step 7: tert-butyl 6-(1-(tert-butoxycarbonyl)piperidin-4-yl)-4-chloro-9H-pyrimido[4,5-b]indole-9-carboxylate

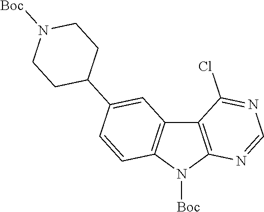 [SMILES: CC(C)(C)OC(=O)N1CCC(c2ccc3c(c2)c2c(Cl)ncnc2n3C(=O)OC(C)(C)C)CC1]

To a stirred solution of 4-chloro-6-(piperidin-4-yl)-9H-pyrimido[4,5-b]indole (410 mg, 1.4 mmol) in NMP (10 mL) was added TEA (723 mg, 7.2 mmol) and Boc₂O (1.2 g, 5.7 mmol) in portions at room temperature under nitrogen atmosphere. The resulting mixture was stirred for 16 h at room temperature. The resulting mixture was diluted with water (50 mL) and extracted with EtOAc (3×50 mL). The combined organic layers were washed with brine (2×30 mL) and dried over anhydrous Na₂SO₄. After filtration, the filtrate was concentrated under reduced pressure. The residue was purified by silica gel column chromatography, eluted with hexane/EtOAc (20:1 to 4:1) to afford the product (200 mg, 28.7%). [M+H]⁺=487.1.

### Step 8: tert-butyl 6-(1-(tert-butoxycarbonyl)piperidin-4-yl)-4-(4-((3-(tert-butyl)-1,2,4-oxadiazole-5-carboxamido)methyl)-3-methylphenyl)-9H-pyrimido[4,5-b]indole-9-carboxylate

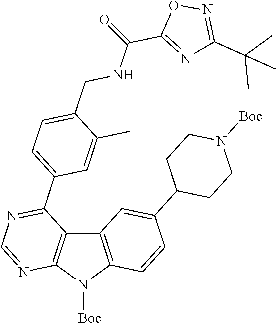 [SMILES: Cc1cc(-c2ncnc3c2c2cc(C4CCN(C(=O)OC(C)(C)C)CC4)ccc2n3C(=O)OC(C)(C)C)ccc1CNC(=O)c1nc(C(C)(C)C)no1]

To a stirred mixture of tert-butyl 6-(1-(tert-butoxycarbonyl)piperidin-4-yl)-4-chloro-9H-pyrimido[4,5-b]indole-9-carboxylate (180 mg, 0.4 mmol), 3-tert-butyl-N-[[2-methyl-4-(4,4,5,5-tetramethyl-1,3,2-dioxaborolan-2-yl)phenyl]methyl]-1,2,4-oxadiazole-5-carboxamide (the compound was obtained through the same way in US20160096834A1) (177 mg, 0.4 mmol) in 1,4-dioxane (5.0 mL) and H₂O (1 mL) were added Pd(dppf)Cl₂ (27 mg, 0.037 mmol) and K₃PO₄ (157 mg, 0.74 mmol) in portions at room temperature. The resulting mixture was stirred for 2 h at 80° C. under nitrogen atmosphere. The resulting mixture was extracted with EtOAc (3×10 mL). The combined organic layers were washed with brine (2×5 mL) and dried over anhydrous Na₂SO₄. After filtration, the filtrate was concentrated under reduced pressure. The residue was purified by reverse phase flash chromatography with the following conditions: column, C18; mobile phase A, 0.1% FA in water, mobile phase B, ACN, 40% to 95% gradient in 25 min; detector, UV 254 nm to afford the product (120 mg, 31.4%). [M+H]⁺=724.5.

### Step 9: 3-(tert-butyl)-N-(2-methyl-4-(6-(piperidin-4-yl)-9H-pyrimido[4,5-b]indol-4-yl)benzyl)-1,2,4-oxadiazole-5-carboxamide

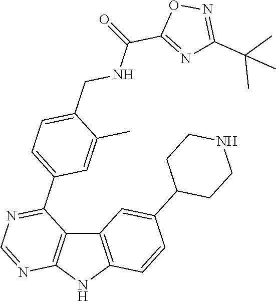 [SMILES: Cc1cc(-c2ncnc3[nH]c4ccc(C5CCNCC5)cc4c23)ccc1CNC(=O)c1nc(C(C)(C)C)no1]

To a stirred solution of tert-butyl 6-(1-(tert-butoxycarbonyl)piperidin-4-yl)-4-(4-((3-(tert-butyl)-1,2,4-oxadiazole-5-carboxamido)methyl)-3-methylphenyl)-9H-pyrimido[4,5-b]indole-9-carboxylate (120 mg, 0.166 mmol) in DCM (2 mL) was added TFA (0.4 mL) at 0° C. The resulting solution was stirred for 1 h at room temperature and concentrated under vacuum. The residue was purified by reverse phase flash chromatography with the following conditions: column, C18; mobile phase A, 0.1% FA in water, mobile phase B, ACN, 10% to 50% gradient in 25 min; detector, UV 254 nm to afford the product (61.3 mg, 64.65%). ¹H NMR (400 MHz, DMSO) δ 12.46 (s, 1H), 10.09 (s, 1H), 8.95 (s, 1H), 8.40 (s, 1H), 7.84-7.72 (m, 3H), 7.55 (dd, J=8.0, 2.4 Hz, 2H), 7.42 (dd, J=8.4, 1.6 Hz, 1H), 4.63 (d, J=4.4 Hz, 2H), 3.22 (d, J=12.0 Hz, 2H), 2.88-2.73 (m, 3H), 2.51 (s, 3H), 1.85 (d, J=13.2 Hz, 2H), 1.64 (q, J=13 Hz, 2H), 1.38 (s, 9H); [M+H]⁺=524.2.

### Step 10: 3-(tert-butyl)-N-(4-(6-(1-((1-(4-(2,4-dioxotetrahydropyrimidin-1(2H)-yl)phenyl)piperidin-4-yl)methyl)piperidin-4-yl)-9H-pyrimido[4,5-b]indol-4-yl)-2-methylbenzyl)-1,2,4-oxadiazole-5-carboxamide

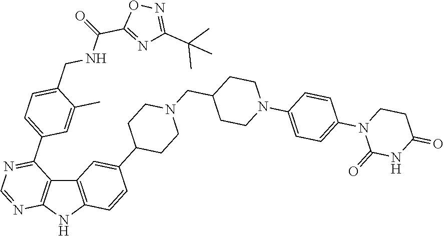 [SMILES: Cc1cc(-c2ncnc3[nH]c4ccc(C5CCN(CC6CCN(c7ccc(N8CCC(=O)NC8=O)cc7)CC6)CC5)cc4c23)ccc1CNC(=O)c1nc(C(C)(C)C)no1]

3-(tert-Butyl)-N-(2-methyl-4-(6-(piperidin-4-yl)-9H-pyrimido[4,5-b]indol-4-yl)benzyl)-1,2,4-oxadiazole-5-carboxamide (50 mg, 0.1 mmol) was dissolved in MeOH/DCM (10:1, 2 mL). 1-(4-(2,4-Dioxotetrahydropyrimidin-1(2H)-yl)phenyl)piperidine-4-carbaldehyde (30.1 mg, 0.1 mmol) was added to the solution in portions and the resulting mixture was stirred at room temperature for 16 h until LC-MS indicated all the starting material was consumed. The mixture was concentrated and purified with SiO₂-gel column (DCM:MeOH=10:1) to give the titled product (29 mg, 35.8%). ¹H NMR (400 MHz, DMSO) δ 12.39 (s, 1H), 11.97 (s, 1H), 10.27 (s, 1H), 9.93 (s, 1H), 8.94 (s, 1H), 7.84 (s, 1H), 7.80-7.78 (m, 2H), 7.58-7.54 (m, 1H), 7.47-7.44 (m, 1H), 7.14-7.12 (m, 2H), 6.96-6.92 (m, 2H), 4.60 (s, 2H), 3.72-3.68 (m, 4H), 3.18-3.05 (m, 1H), 3.05-2.72 (m, 2H), 2.70-2.66 (m, 4H), 2.47 (s, 3H), 2.19-2.17 (m, 2H), 2.15-1.95 (m, 2H), 1.92-1.90 (m, 2H), 1.84-1.78 (m, 3H), 1.75-1.70 (m, 2H), 1.38 (s, 9H), 1.25-1.22 (m, 2H); [M+H]⁺=809.7.

### Example 3: 3-(tert-butyl)-N-(4-(7-(1-(3-(4-((2,6-dioxopiperidin-3-yl)amino)phenoxy)propyl)piperidin-4-yl)-9H-pyrimido[4,5-b]indol-4-yl)-2-methylbenzyl)-1,2,4-oxadiazole-5-carboxamide

### Step 1: ethyl 2-(4-bromo-2-nitrophenyl)-2-cyanoacetate

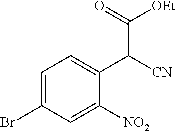 [SMILES: CCOC(=O)C(C#N)c1ccc(Br)cc1[N+](=O)[O-]]

To a stirred mixture of NaH (10.2 g, 253.8 mmol, 60%) in DMF (200 mL) was added ethyl cyanoacetate (15 mL) in DMF (150 mL) dropwise at 0° C. under nitrogen atmosphere. The resulting mixture was stirred for 0.5 h at 0° C. under nitrogen atmosphere. To the above mixture was added 4-bromo-1-chloro-2-nitrobenzene (15 g, 63.4 mmol) at 0° C. The resulting mixture was stirred for additional 4 h at 70° C. The mixture was cooled down to room temperature and acidified to pH 6 with HCl (aq.) (1 M). The resulting mixture was extracted with EtOAc (3×300 mL). The combined organic layers were washed with brine (3×100 mL) and dried over anhydrous Na₂SO₄. After filtration, the filtrate was concentrated under reduced pressure. The residue was purified by silica gel column chromatography, eluted with hexane/EtOAc (1:1) to afford the product (14.5 g, 73%). [M−H]⁻=311.0.

### Step 2: ethyl 2-amino-6-bromo-1H-indole-3-carboxylate

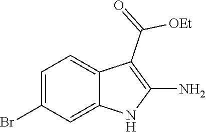 [SMILES: CCOC(=O)c1c(N)[nH]c2cc(Br)ccc12]

A mixture of ethyl 2-(4-bromo-2-nitrophenyl)-2-cyanoacetate (14 g, 44.7 mmol), Fe (25.0 g, 447.1 mmol) and NH₄Cl (2.4 g, 44.7 mmol) in EtOH (140 mL) was stirred for 2 h at 80° C. The mixture was cooled down to room temperature. The resulting mixture was filtered and the filter cake was washed with MeOH (2×100 mL). The filtrate was concentrated under reduced pressure. The resulting mixture was extracted with EtOAc (3×200 mL). The combined organic layers were washed with brine (2×100 mL) and dried over anhydrous Na₂SO₄. After filtration, the filtrate was concentrated under reduced pressure. The residue was purified by silica gel column chromatography, eluted with hexane/EtOAc (1:1) to afford the product (6.7 g, 52.0%). [M+H]⁺=283.0.

### Step 3: 7-bromo-3,9-dihydro-4H-pyrimido[4,5-b]indol-4-one

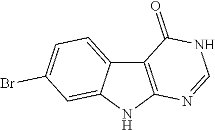 [SMILES: O=c1[nH]cnc2[nH]c3cc(Br)ccc3c12]

A mixture of ethyl 2-amino-6-bromo-1H-indole-3-carboxylate (3 g, 10.6 mmol) and formamidine acetate (4.4 g, 42.4 mmol) in 2-methoxyethanol (30 mL) was stirred for 24 h at 125° C. The mixture was cooled down to room temperature. To the resulting mixture was added water and filtered. The filter cake was washed with water (3×20 mL). The solid was dried under infrared light to give the product (2.5 g, 85%). [M+H]⁺=264.0.

### Step 4: 7-bromo-4-chloro-9H-pyrimido[4,5-b]indole

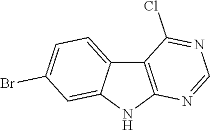 [SMILES: Clc1ncnc2[nH]c3cc(Br)ccc3c12]

A solution of 7-bromo-3,9-dihydro-4H-pyrimido[4,5-b]indol-4-one (2.5 g, 9.467 mmol) in POCl₃ (25 mL) and dioxane (25 mL) was stirred for 1 h at 100° C. The resulting mixture was concentrated under vacuum. The mixture was basified to pH 8 with sat. NaHCO₃ and extracted with EtOAc (3×50 mL). The combined organic layers were washed with brine (50 mL) and dried over anhydrous Na₂SO₄. After filtration, the filtrate was concentrated under reduced pressure. The solid was collected by filtration and washed with diethyl ether (2×30 mL) to afford the product (2.6 g, 97%). [M+H]⁺=282.0.

### Step 5: tert-butyl 4-(4-chloro-9H-pyrimido[4,5-b]indol-7-yl)-3,6-dihydropyridine-1(2H)-carboxylate

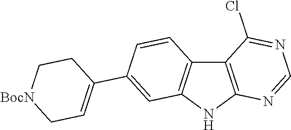 [SMILES: CC(C)(C)OC(=O)N1CC=C(c2ccc3c(c2)[nH]c2ncnc(Cl)c23)CC1]

To a stirred mixture of 7-bromo-4-chloro-9H-pyrimido[4,5-b]indole (2.4 g, 8.5 mmol) and tert-butyl 4-(4,4,5,5-tetramethyl-1,3,2-dioxaborolan-2-yl)-3,6-dihydro-2H-pyridine-1-carboxylate (2.4 g, 7.6 mmol) in dioxane (50 mL) and H₂O (10 mL) were added Pd(dppf)Cl₂ (621 mg, 0.8 mmol) and K₃PO₄ (3.6 g, 17.0 mmol) at room temperature under nitrogen atmosphere. The resulting mixture was stirred for 16 h at 80° C. under nitrogen atmosphere. The mixture was cooled down to room temperature. To the resulting mixture was added water (10 mL) and extracted with EtOAc (3×80 mL). The combined organic layers were washed with brine (10 mL) and dried over anhydrous Na₂SO₄. After filtration, the filtrate was concentrated under reduced pressure. The residue was purified by silica gel column chromatography, eluted with hexane/EtOAc (30%-80%) to afford the product (1.8 g, 55%). [M+H]⁺=385.2.

### Step 6: benzyl (4-bromo-2-methylbenzyl)carbamate

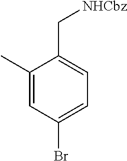 [SMILES: Cc1cc(Br)ccc1CNC(=O)OCc1ccccc1]

To a stirred solution of 1-(4-bromo-2-methylphenyl)methanamine (10 g, 49.980 mmol) in THF (100 mL) and H₂O (100 mL) was added NaOH (2.1 g, 52.479 mmol) at 0° C. To the above solution was added benzyl chloroformate (8.95 g, 52.479 mmol) dropwise at 0° C. The resulting solution was stirred for additional 16 h at room temperature. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by silica gel column chromatography, eluted with hexane/EtOAc (3:1) to afford the product (6.7 g, 40%). [M+H]⁺=335.0.

### Step 7: benzyl (2-methyl-4-(4,4,5,5-tetramethyl-1,3,2-dioxaborolan-2-yl)benzyl)carbamate

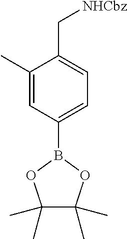

To a stirred solution of benzyl (4-bromo-2-methylbenzyl)carbamate (6.5 g, 19.4 mmol) and bis(pinacolato)diboron (5.9 g, 23.3 mmol) in 1,4-dioxane (65 mL) were added Cs₂CO₃ (12.7 g, 38.9 mmol) and Pd(dppf)Cl₂ (1.4 g, 1.9 mmol) at room temperature under nitrogen atmosphere. The resulting mixture was stirred for 16 h at 80° C. under nitrogen atmosphere. The mixture was cooled down to room temperature. To the resulting mixture was added water (30 mL) and extracted with EtOAc (3×80 mL). The combined organic layers were washed with brine (10 mL) and dried over anhydrous Na₂SO₄. After filtration, the filtrate was concentrated under reduced pressure. The residue was purified by silica gel column chromatography, eluted with hexane/EtOAc(30%-80% EtOAc) to afford the product (5.7 g, 76.0%). [M+H]⁺=382.2.

### Step 8: tert-butyl 4-(4-(4-((((benzyloxy)carbonyl)amino)methyl)-3-methylphenyl)-9H-pyrimido[4,5-b]indol-7-yl)-3,6-dihydropyridine-1(2H)-carboxylate

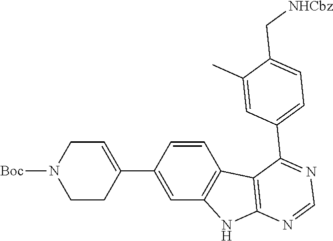 [SMILES: Cc1cc(-c2ncnc3[nH]c4cc(C5=CCN(C(=O)OC(C)(C)C)CC5)ccc4c23)ccc1CNC(=O)OCc1ccccc1]

To a stirred solution of tert-butyl 4-(4-chloro-9H-pyrimido[4,5-b]indol-7-yl)-3,6-dihydropyridine-1(2H)-carboxylate (1.8 g, 4.7 mmol) and benzyl (2-methyl-4-(4,4,5,5-tetramethyl-1,3,2-dioxaborolan-2-yl)benzyl)carbamate (2.1 g, 5.6 mmol) in dioxane (40 mL) and H₂O (8 mL) were added K₃PO₄ (1.0 g, 9.4 mmol) and Pd(dppf)Cl₂ (342 mg, 0.5 mmol) at room temperature under nitrogen atmosphere. The resulting mixture was stirred for 16 h at 80° C. under nitrogen atmosphere. The mixture was cooled down to room temperature. To the resulting mixture was added water (30 mL) and extracted with EtOAc (3×80 mL). The combined organic layers were washed with brine (10 mL) and dried over anhydrous Na₂SO₄. After filtration, the filtrate was concentrated under reduced pressure. The residue was purified by silica gel column chromatography, eluted with DCM/MeOH (0%-10% MeOH) to afford the product (1.6 g, 56%). [M+H]⁺=604.3.

### Step 9: tert-butyl 4-[4-[4-(aminomethyl)-3-methylphenyl]-9H-pyrimido[4,5-b]indol-7-yl]piperidine-1-carboxylate

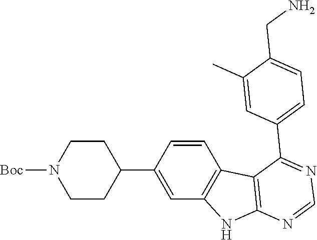 [SMILES: Cc1cc(-c2ncnc3[nH]c4cc(C5CCN(C(=O)OC(C)(C)C)CC5)ccc4c23)ccc1CN]

To a stirred solution of tert-butyl 4-(4-(4-((((benzyloxy)carbonyl)amino)methyl)-3-methylphenyl)-9H-pyrimido[4,5-b]indol-7-yl)-3,6-dihydropyridine-1(2H)-carboxylate (1.6 g, 2.7 mmol) in EtOH (16 mL), EtOAc (7 mL) and DCM (16 mL) was added Pd/C (2.8 g, 26.5 mmol) at room temperature under nitrogen atmosphere. The final reaction mixture was stirred for 48 h at room temperature under H₂ (2 MPa) atmosphere. The resulting mixture was filtered and the filter cake was washed with MeOH (3×80 mL). The filtrate was concentrated under reduced pressure to give the product (1.2 g, 96%). [M+H]⁺=472.4.

### Step 10: tert-butyl 4-(4-(4-((3-(tert-butyl)-1,2,4-oxadiazole-5-carboxamido)methyl)-3-methylphenyl)-9H-pyrimido[4,5-b]indol-7-yl)piperidine-1-carboxylate

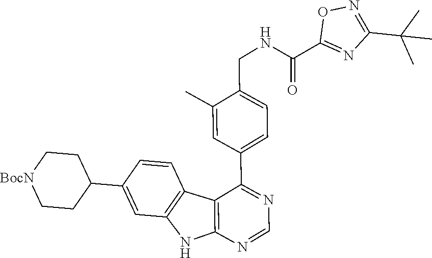 [SMILES: Cc1cc(-c2ncnc3[nH]c4cc(C5CCN(C(=O)OC(C)(C)C)CC5)ccc4c23)ccc1CNC(=O)c1nc(C(C)(C)C)no1]

To a stirred solution of 3-tert-butyl-1,2,4-oxadiazole-5-carboxylic acid (840 mg, 4.9 mmol) and tert-butyl 4-[4-[4-(aminomethyl)-3-methylphenyl]-9H-pyrimido[4,5-b]indol-7-yl]piperidine-1-carboxylate (540 mg, 1.1 mmol) in DCM (8 mL) were added DCC (2.0 g, 9.9 mmol) and DMAP (603 mg, 4.9 mmol) at room temperature. The resulting mixture was stirred for 16 h at room temperature and diluted with water (70 mL). The resulting mixture was extracted with EtOAc (3×120 mL). The combined organic layers were washed with brine (2×100 mL) and dried over anhydrous Na₂SO₄. After filtration, the filtrate was concentrated under reduced pressure. The residue was purified by silica gel column chromatography, eluted with 0-10% (MeOH in DCM) to afford the product (410 mg, 13%). [M+H]⁺=624.3.

### Step 11: 3-(tert-butyl)-N-(2-methyl-4-(7-(piperidin-4-yl)-9H-pyrimido[4,5-b]indol-4-yl)benzyl)-1,2,4-oxadiazole-5-carboxamide

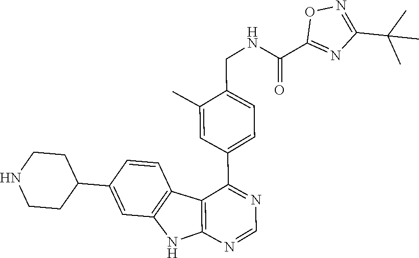 [SMILES: Cc1cc(-c2ncnc3[nH]c4cc(C5CCNCC5)ccc4c23)ccc1CNC(=O)c1nc(C(C)(C)C)no1]

A mixture of tert-butyl 4-(4-(4-((3-(tert-butyl)-1,2,4-oxadiazole-5-carboxamido)methyl)-3-methylphenyl)-9H-pyrimido[4,5-b]indol-7-yl)piperidine-1-carboxylate (410 mg, 0.7 mmol) in TFA (1 mL) and DCM (4 mL) was stirred for 2 h at room temperature. The resulting mixture was concentrated under reduced pressure. The residue was purified by reverse phase flash chromatography with the following conditions: column, C18 silica gel; mobile phase, MeCN in water (0.1% FA), 10% to 50% gradient in 15 min; detector, UV 254 nm to afford the product (171.6 mg, 45%). ¹H NMR (400 MHz, DMSO) δ 12.47 (s, 1H), 9.93 (t, J=6.0 Hz, 1H), 8.95 (s, 1H), 8.29 (s, 1H), 7.86-7.69 (m, 3H), 7.53 (d, J=8.4 Hz, 1H), 7.41 (s, 1H), 7.10 (d, J=8.0 Hz, 1H), 4.61 (d, J=6.0 Hz, 2H), 2.98-2.88 (m, 3H), 2.55 (s, 2H), 2.47 (s, 3H), 1.98-1.89 (m, 2H), 1.84-1.75 (m, 2H), 1.39 (s, 9H); [M+H]⁺=524.3.

### Step 12: 3-(4-((2,6-dioxopiperidin-3-yl)amino)phenoxy)propyl 4-methylbenzenesulfonate

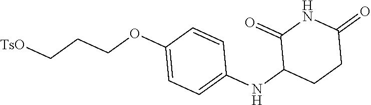 [SMILES: Cc1ccc(S(=O)(=O)OCCCOc2ccc(NC3CCC(=O)NC3=O)cc2)cc1]

3-((4-(3-Hydroxypropoxy)phenyl)amino)piperidine-2,6-dione (100 mg, 0.36 mmol) and DIEA (93 mg, 0.72 mmol) were dissolved in DCM (2 mL). TsCl (138 mg, 0.72 mmol) was added to the solution in portions at 0° C. and the resulting mixture was stirred at room temperature for 16 h until LC-MS indicated all the starting material was consumed. The resulting mixture was slowly added to ice water and extracted with EtOAc for 3 times. The combined organic layer was dried over anhydrous Na₂SO₄, concentrated, and purified with SiO₂-gel column (eluted with DCM/MeOH=95:5) to give the desired product (52 mg, 33%). [M+H]⁺=433.2.

### Step 13: 3-(tert-butyl)-N-(4-(7-(1-(3-(4-((2,6-dioxopiperidin-3-yl)amino)phenoxy)propyl)piperidin-4-yl)-9H-pyrimido[4,5-b]indol-4-yl)-2-methylbenzyl)-1,2,4-oxadiazole-5-carboxamide

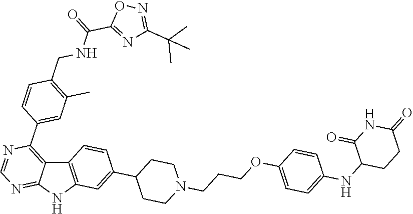 [SMILES: Cc1cc(-c2ncnc3[nH]c4cc(C5CCN(CCCOc6ccc(NC7CCC(=O)NC7=O)cc6)CC5)ccc4c23)ccc1CNC(=O)c1nc(C(C)(C)C)no1]

3-(tert-Butyl)-N-(2-methyl-4-(7-(piperidin-4-yl)-9H-pyrimido[4,5-b]indol-4-yl)benzyl)-1,2,4-oxadiazole-5-carboxamide (52 mg, 0.1 mmol), 3-(4-((2,6-dioxopiperidin-3-yl)amino)phenoxy)propyl 4-methylbenzenesulfonate (50 mg, 0.12 mmol) and DIEA (31 mg, 0.24 mmol) were dissolved in MeCN (5 mL). The resulting mixture was stirred at 80° C. for 16 h until LC-MS indicated all the starting material was consumed. The mixture was concentrated and purified with prep-HPLC to provide the product (6 mg, 7.7%). ¹H NMR (400 MHz, DMSO) δ 12.40 (s, 1H), 10.77 (s, 1H), 9.92 (s, 1H), 8.92 (s, 1H), 8.37-8.35 (m, 2H), 7.78-7.75 (m, 2H), 7.54-7.52 (m, 1H), 7.41 (s, 1H), 7.15-7.12 (m, 1H), 6.73 (d, J=8.0 Hz, 2H), 6.64 (d, J=8.0 Hz, 2H), 5.46-5.44 (m, 1H), 4.61 (s, 2H), 4.25-4.20 (m, 1H), 3.92-3.88 (m, 2H), 3.21-3.15 (m, 2H), 3.15-3.05 (m, 2H), 3.05-2.95 (m, 2H), 2.50 (s, 3H), 2.18-2.08 (m, 2H), 1.93-1.82 (m, 1H), 1.77-1.72 (m, 2H), 1.72-1.66 (m, 3H), 1.38 (s, 9H); [M+H]⁺=784.4.

### Example 4: 3-(tert-butyl)-N-(4-(7-(1-((1-(4-(2,4-dioxotetrahydropyrimidin-1(2H)-yl)phenyl)piperidin-4-yl)methyl)piperidin-4-yl)-9H-pyrimido[4,5-b]indol-4-yl)-2-methylbenzyl)-1,2,4-oxadiazole-5-carboxamide

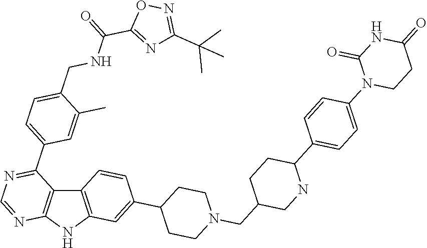 [SMILES: Cc1cc(-c2ncnc3[nH]c4cc(C5CCN(CC6CCC(c7ccc(N8CCC(=O)NC8=O)cc7)NC6)CC5)ccc4c23)ccc1CNC(=O)c1nc(C(C)(C)C)no1]

3-(tert-Butyl)-N-(2-methyl-4-(7-(piperidin-4-yl)-9H-pyrimido[4,5-b]indol-4-yl)benzyl)-1,2,4-oxadiazole-5-carboxamide (100 mg, 0.19 mmol) was dissolved in MeOH/DCM (10:1, 2 mL). 1-(4-(2,4-Dioxotetrahydropyrimidin-1(2H)-yl)phenyl)piperidine-4-carbaldehyde (57.2 mg, 0.19 mmol) was added to the solution in portions and the resulting mixture was stirred at room temperature for 16 h until LC-MS indicated all the starting material was consumed. The mixture was concentrated and purified with SiO₂-gel column (DCM:MeOH=10:1) to give the titled product (73 mg, 47.6%). ¹H NMR (400 MHz, DMSO) δ 12.41 (s, 1H), 11.97 (s, 1H), 10.27 (s, 1H), 9.93 (s, 1H), 8.94 (s, 1H), 7.78-7.74 (m, 2H), 7.53-7.51 (m, 1H), 7.42 (s, 1H), 7.14-7.12 (m, 3H), 6.96-6.92 (m, 2H), 4.60 (s, 2H), 3.72-3.68 (m, 4H), 3.18-3.05 (m, 1H), 3.05-2.72 (m, 2H), 2.70-2.66 (m, 4H), 2.47 (s, 3H), 2.21-2.18 (m, 2H), 2.15-1.95 (m, 2H), 1.92-1.90 (m, 2H), 1.84-1.78 (m, 3H), 1.75-1.70 (m, 2H), 1.38 (s, 9H), 1.25-1.22 (m, 2H); [M+H]⁺=810.4.

### Example 5: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrimido[4,5-b]indol-4-yl)-2-methylphenyl)ethyl)-1,2,4-oxadiazole-5-carboxamide

### Step 1: tert-butyl 4-[4-oxo-3H,9H-pyrimido[4,5-b]indol-7-yl]-3,6-dihydro-2H-pyridine-1-carboxylate

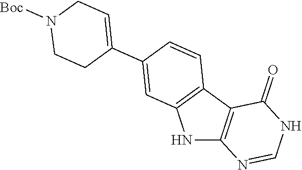 [SMILES: CC(C)(C)OC(=O)N1CC=C(c2ccc3c(c2)[nH]c2nc[nH]c(=O)c23)CC1]

To a stirred mixture of 7-bromo-3,9-dihydro-4H-pyrimido[4,5-b]indol-4-one (16.5 g, 62.5 mmol) and tert-butyl 4-(4,4,5,5-tetramethyl-1,3,2-dioxaborolan-2-yl)-3,6-dihydro-2H-pyridine-1-carboxylate (29.0 g, 93.7 mmol) in dioxane (300 mL) and H₂O (60 mL) were added Pd(dppf)Cl₂ (4.6 g, 6.2 mmol) and K₃PO₄ (26.5 g, 125.0 mmol) at room temperature under nitrogen atmosphere. The resulting mixture was stirred for 16 h at 80° C. under nitrogen atmosphere. The mixture was cooled down to room temperature. To the resulting mixture was added water (100 mL) and extracted with EtOAc (3×300 mL). The combined organic layers were washed with brine (100 mL) and dried over anhydrous Na₂SO₄. After filtration, the filtrate was concentrated under reduced pressure. The residue was purified by silica gel column chromatography, eluted with MeOH in DCM (0-10%) to afford the product (16 g, 69.9%). [M+H]⁺=367.3.

### Step 2: tert-butyl 4-(4-oxo-4,9-dihydro-3H-pyrimido[4,5-b]indol-7-yl)piperidine-1-carboxylate

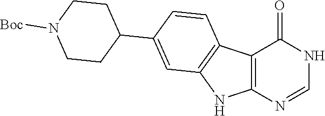 [SMILES: CC(C)(C)OC(=O)N1CCC(c2ccc3c(c2)[nH]c2nc[nH]c(=O)c23)CC1]

To a stirred solution of tert-butyl 4-[4-oxo-3H,9H-pyrimido[4,5-b]indol-7-yl]-3,6-dihydro-2H-pyridine-1-carboxylate (16 g, 43.7 mmol) in DCM (500 mL), EtOAc (250 mL) and EtOH (500 mL) was added Pd/C (30 g) at room temperature under nitrogen atmosphere. The final reaction mixture was stirred for 48 h at room temperature under H₂ (2 MPa) atmosphere. The resulting mixture was filtered and the filter cake was washed with MeOH (3×80 mL). The filtrate was concentrated under reduced pressure to give the product (14 g, 60.9%). [M+H]⁺=369.1.

### Step 3: 4-chloro-7-(piperidin-4-yl)-9H-pyrimido[4,5-b]indole

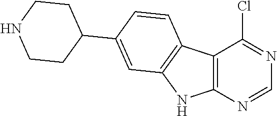 [SMILES: Clc1ncnc2[nH]c3cc(C4CCNCC4)ccc3c12]

A solution of tert-butyl 4-(4-oxo-4,9-dihydro-3H-pyrimido[4,5-b]indol-7-yl)piperidine-1-carboxylate (10 g, 27.1 mmol) in POCl₃ (75.9 mL, 814.3 mmol) and dioxane (200 mL) was stirred for 16 h at 110° C. The resulting mixture was concentrated under vacuum. The mixture was basified to pH=8 with sat. NaHCO₃ (aq.). The product was collected by filtration and washed with water (2×50 mL). The solid was dried under infrared light to give the product (10.5 g, crude). [M+H]⁺=287.2.

### Step 4: tert-butyl 7-(1-(tert-butoxycarbonyl)piperidin-4-yl)-4-chloro-9H-pyrimido[4,5-b]indole-9-carboxylate

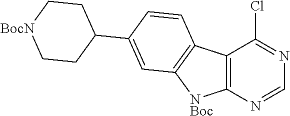 [SMILES: CC(C)(C)OC(=O)N1CCC(c2ccc3c4c(Cl)ncnc4n(C(=O)OC(C)(C)C)c3c2)CC1]

To a stirred solution of 4-chloro-7-(piperidin-4-yl)-9H-pyrimido[4,5-b]indole (10 g, 34.9 mmol) in NMP (40 mL) and DCM (200 mL) were added TEA (24 mL, 239.5 mmol), DMAP (426 mg, 3.5 mmol) and Boc₂O (22 mL, 102.6 mmol) at room temperature under nitrogen atmosphere. The resulting mixture was stirred for 16 h at room temperature and extracted with DCM (3×200 mL). The combined organic layers were washed with brine (2×50 mL) and dried over anhydrous Na₂SO₄. After filtration, the filtrate was concentrated under reduced pressure. The residue was purified by silica gel column chromatography, eluted with EtOAc in petroleum ether (1-10%) to afford the product (1.3 g, 7.6%). [M+H]⁺=487.2.

### Step 5: tert-butyl (R)-7-(1-(tert-butoxycarbonyl)piperidin-4-yl)-4-(4-(1-(3-(tert-butyl)-1,2,4-oxadiazole-5-carboxamido)ethyl)-3-methylphenyl)-9H-pyrimido[4,5-b]indole-9-carboxylate

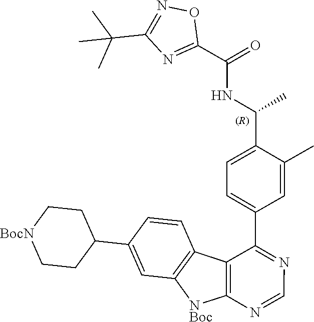

To a stirred mixture of 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (the compound was obtained through the same way in WO2021219070A) (2.0 g, 4.9 mmol) and tert-butyl 7-(1-(tert-butoxycarbonyl)piperidin-4-yl)-4-chloro-9H-pyrimido[4,5-b]indole-9-carboxylate (2 g, 4.1 mmol) in dioxane (20 mL) and H₂O (4 mL) were added Pd(dppf)Cl₂ (300 mg, 0.4 mmol) and K₃PO₄ (1.7 g, 8.2 mmol) at room temperature under nitrogen atmosphere. The resulting mixture was stirred for 16 h at 80° C. under nitrogen atmosphere. The mixture was cooled down to room temperature. To the resulting mixture was added water (20 mL) and extracted with EtOAc (3×50 mL). The combined organic layers were washed with brine (20 mL) and dried over anhydrous Na₂SO₄. After filtration, the filtrate was concentrated under reduced pressure. The residue was purified by silica gel column chromatography, eluted with EtOAc/petroleum ether (0-40%) to afford the product (2.7 g, 89.1%). [M+H]⁺=738.3.

### Step 6: (R)-3-(tert-butyl)-N-(1-(2-methyl-4-(7-(piperidin-4-yl)-9H-pyrimido[4,5-b]indol-4-yl)phenyl)ethyl)-1,2,4-oxadiazole-5-carboxamide

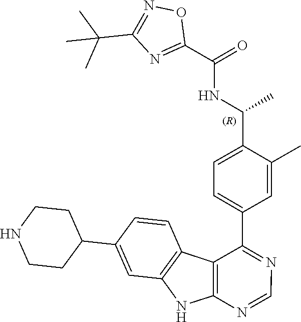

A mixture of tert-butyl (R)-7-(1-(tert-butoxycarbonyl)piperidin-4-yl)-4-(4-(1-(3-(tert-butyl)-1,2,4-oxadiazole-5-carboxamido)ethyl)-3-methylphenyl)-9H-pyrimido[4,5-b]indole-9-carboxylate (2.8 g, 3.8 mmol) in TFA (7 mL) and DCM (28 mL) was stirred for 2 h at room temperature. The resulting mixture was concentrated under reduced pressure. The residue was purified by reverse phase flash chromatography with the following conditions: column, C18 silica gel; mobile phase, MeCN in water (0.1% FA), 10% to 50% gradient in 15 min; detector, UV 254 nm to afford the product (1.7 g, 83.1%). ¹H NMR (300 MHz, DMSO) δ 12.47 (s, 1H), 9.93 (d, J=7.8 Hz, 1H), 8.94 (s, 1H), 7.85-7.66 (m, 4H), 7.40 (s, 1H), 7.11 (d, J=8.4 Hz, 1H), 5.42 (t, J=7.5 Hz, 1H), 3.40 (d, J=12.0 Hz, 2H), 3.10-2.96 (m, 3H), 2.50-2.48 (m, 3H), 2.00 (d, J=13.2 Hz, 2H), 1.92-1.76 (m, 2H), 1.58 (d, J=6.9 Hz, 3H), 1.37 (s, 9H). [M+H]⁺=538.35.

### Step 7: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrimido[4,5-b]indol-4-yl)-2-methylphenyl)ethyl)-1,2,4-oxadiazole-5-carboxamide

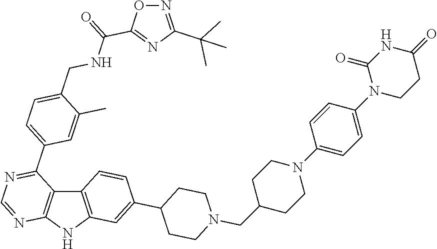 [SMILES: Cc1cc(-c2ncnc3[nH]c4cc(C5CCN(CC6CCN(c7ccc(N8CCC(=O)NC8=O)cc7)CC6)CC5)ccc4c23)ccc1CNC(=O)c1nc(C(C)(C)C)no1]

(R)-3-(tert-Butyl)-N-(1-(2-methyl-4-(7-(piperidin-4-yl)-9H-pyrimido[4,5-b]indol-4-yl)phenyl)ethyl)-1,2,4-oxadiazole-5-carboxamide (100 mg, 0.19 mmol) was dissolved in MeOH/DCM (10:1, 2 mL). 1-(4-(2,4-Dioxotetrahydropyrimidin-1(2H)-yl)phenyl) piperidine-4-carbaldehyde (57.2 mg, 0.19 mmol) was added to the solution in portions and the resulting mixture was stirred at room temperature for 16 h until LC-MS indicated all the starting material was consumed. The mixture was concentrated and purified with SiO₂-gel column (DCM:MeOH=10:1) to give the titled product (98 mg, 42.7%). ¹H NMR (400 MHz, DMSO) δ 12.07 (s, 1H), 10.34 (s, 1H), 10.04 (s, 1H), 9.01 (s, 1H), 7.78-7.56 (m, 4H), 7.37 (s, 1H), 7.14-7.12 (m, 3H), 6.96-6.92 (m, 2H), 5.68 (s, 1H), 3.72-3.68 (m, 4H), 3.18-2.92 (m, 4H), 2.61-2.54 (m, 4H), 2.47 (s, 3H), 2.21-2.18 (m, 2H), 2.15-1.95 (m, 2H), 1.92-1.90 (m, 3H), 1.84-1.78 (m, 2H), 1.78-1.76 (m, 4H), 1.38 (s, 9H), 1.25-1.22 (m, 2H); [M+H]⁺=823.5.

### Example 6: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2-methylphenyl)ethyl)-1,2,4-oxadiazole-5-carboxamide

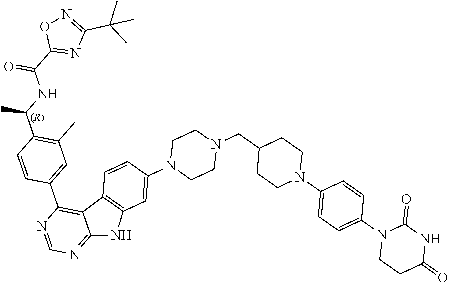

(R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide (the compound was obtained through the similar way in example 5) (100 mg, 0.19 mmol) was dissolved in MeOH/DCM (10:1, 2 mL). 1-(4-(2,4-Dioxotetrahydropyrimidin-1(2H)-yl)phenyl) piperidine-4-carbaldehyde (57.2 mg, 0.19 mmol) was added to the solution in portions and the resulting mixture was stirred at room temperature for 16 h until LC-MS indicated all the starting material was consumed. The mixture was concentrated and purified with SiO₂-gel column (DCM:MeOH=10:1) to give the titled product (58 mg, 37.1%). ¹H NMR (400 MHz, DMSO) δ 12.21 (s, 1H), 10.27 (s, 1H), 9.96 (s, 1H), 8.83 (s, 1H), 7.81-7.71 (m, 4H), 7.14 (d, J=8.0 Hz, 2H), 6.96-6.92 (m, 4H), 5.42 (s, 1H), 3.72-3.68 (m, 4H), 3.18-2.92 (m, 4H), 2.75-2.65 (m, 5H), 2.61-2.55 (m, 2H), 2.47 (s, 3H), 2.21-2.18 (m, 1H), 2.15-1.95 (m, 1H), 1.87-1.68 (m, 3H), 1.60-1.58 (m, 3H), 1.37 (s, 9H), 1.25-1.20 (m, 3H); [M+H]⁺=824.5.

### Example 7: (R)-3-(tert-butyl)-N-(1-(4-(7-(4-((1-(5-(2,4-dioxotetrahydropyrimidin-1(2H)-yl)pyridin-2-yl)piperidin-4-yl)methyl)piperazin-1-yl)-9H-pyrimido[4,5-b]indol-4-yl)-2-methylphenyl)ethyl)-1,2,4-oxadiazole-5-carboxamide

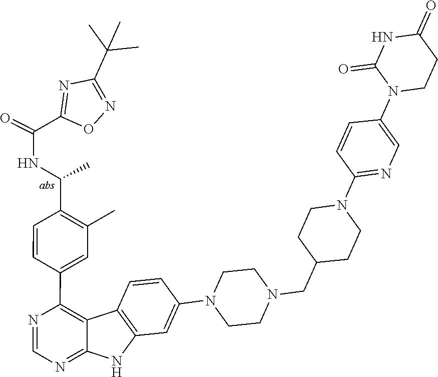 [SMILES: Cc1cc(-c2ncnc3[nH]c4cc(N5CCN(CC6CCN(c7ccc(N8CCC(=O)NC8=O)cn7)CC6)CC5)ccc4c23)ccc1[C@@H](C)NC(=O)c1nc(C(C)(C)C)no1]

A mixture of (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide hydrobromide (200 mg, 0.33 mmol) and 1-(5-(2,4-dioxotetrahydropyrimidin-1(2H)-yl)pyridin-2-yl)piperidine-4-carbaldehyde (110 mg, 0.364 mmol) in MeOH (10 mL) was stirred in a round bottom flask for 30 min at room temperature. Then NaBH(OAc)₃ (140 mg, 0.66 mmol) was added and stirred for 2 h at room temperature. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (5 mg, 1.8%). ¹H NMR (400 MHz, DMSO) δ 12.17 (s, 1H), 10.31 (s, 1H), 9.94 (d, J=7.6 Hz, 1H), 8.80 (s, 1H), 7.97 (d, J=2.8 Hz, 1H), 7.79-7.75 (m, 1H), 7.72 (s, 1H), 7.70-7.64 (m, 2H), 7.42 (dd, J=8.8, 2.8 Hz, 1H), 6.93-6.88 (m, 2H), 6.41 (d, J=9.2 Hz, 1H), 5.44-5.34 (m, 1H), 3.70-3.57 (m, 4H), 3.52-3.49 (m, 1H), 3.27-3.20 (m, 5H), 3.02-2.91 (m, 1H), 2.67 (t, J=6.8 Hz, 2H), 2.54 (s, 5H), 2.60-2.50 (m, 5H), 2.45-2.35 (m, 3H), 2.35-2.20 (m, 2H), 2.18-2.05 (m, 2H), 1.68-1.58 (m, 4H), 1.56 (d, J=7.2 Hz, 4H), 1.35 (s, 9H); [M+H]⁺=825.5.

### Example 8: (R)-3-(tert-butyl)-N-(1-(4-(7-(4-((1-(4-(2,4-dioxotetrahydropyrimidin-1(2H)-yl)-3-methylphenyl)piperidin-4-yl)methyl)piperazin-1-yl)-9H-pyrimido[4,5-b]indol-4-yl)-2-methylphenyl)ethyl)-1,2,4-oxadiazole-5-carboxamide

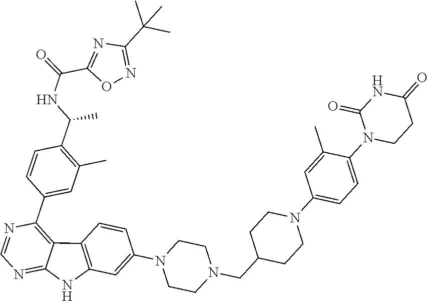 [SMILES: Cc1cc(-c2ncnc3[nH]c4cc(N5CCN(CC6CCN(c7ccc(N8CCC(=O)NC8=O)c(C)c7)CC6)CC5)ccc4c23)ccc1[C@@H](C)NC(=O)c1nc(C(C)(C)C)no1]

A mixture of (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide hydrobromide (200 mg, 0.33 mmol) and 1-(4-(2,4-dioxotetrahydropyrimidin-1(2H)-yl)-3-methylphenyl)piperidine-4-carbaldehyde (115 mg, 0.364 mmol) in MeOH (10 mL) was stirred in a round bottom flask for 30 min at room temperature. Then NaBH(OAc)₃ (140 mg, 0.66 mmol) was added and stirred for 2 h at room temperature. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (105 mg, 38%). ¹H NMR (400 MHz, DMSO) δ 12.18 (s, 1H), 10.24 (s, 2H), 9.94 (d, J=7.6 Hz, 2H), 8.80 (s, 1H), 7.80-7.61 (m, 4H), 7.09-6.86 (m, 3H), 6.85-6.73 (m, 2H), 5.44-5.36 (m, 1H), 3.80-3.55 (m, 4H), 3.47-3.40 (m, 1H), 3.30-3.20 (m, 4H), 2.75-2.60 (m, 5H), 2.55-2.50 (m, 3H), 2.25-2.18 (m, 1H), 2.10 (s, 3H), 1.84-1.75 (m, 1H), 1.75-1.65 (m, 1H), 1.56 (d, J=7.2 Hz, 2H), 1.35 (s, 9H); [M+H]⁺=838.6.

### Example 9: (R)-3-(tert-butyl)-N-(1-(4-(7-(1-((1-(4-(2,4-dioxotetrahydropyrimidin-1(2H)-yl)-3-fluorophenyl)piperidin-4-yl)methyl)piperidin-4-yl)-9H-pyrimido[4,5-b]indol-4-yl)-2-methylphenyl)ethyl)-1,2,4-oxadiazole-5-carboxamide

### Step 1: 1-(4-(2,4-dioxotetrahydropyrimidin-1(2H)-yl)-3-fluorophenyl)piperidine-4-carbaldehyde

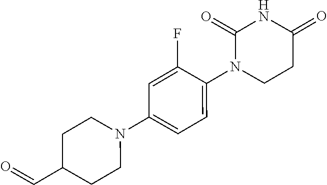 [SMILES: O=CC1CCN(c2ccc(N3CCC(=O)NC3=O)c(F)c2)CC1]

1-(2-fluoro-4-(4-(hydroxymethyl)piperidin-1-yl)phenyl)dihydropyrimidine-2,4(1H,3H)-dione (200 mg, 0.62 mmol) was placed in DMSO (5 mL) and IBX (349 mg, 1.2 mmol) was added to the solution in portions at room temperature. The resulting mixture was stirred at room temperature for 16 h until TLC indicated all the starting material was consumed. The mixture was diluted with water (25 mL) and extracted with EtOAc (25 mL*3). The combined organic layer was washed with brine (50 mL*2), dried over Na₂SO₄ and concentrated to provide the desired product (160 mg, crude) which was used directly without further purification. [M+H]⁺=320.2.

### Step 2: (R)-3-(tert-butyl)-N-(1-(4-(7-(1-((1-(4-(2,4-dioxotetrahydropyrimidin-1(2H)-yl)-3-fluorophenyl)piperidin-4-yl)methyl)piperidin-4-yl)-9H-pyrimido[4,5-b]indol-4-yl)-2-methylphenyl)ethyl)-1,2,4-oxadiazole-5-carboxamide

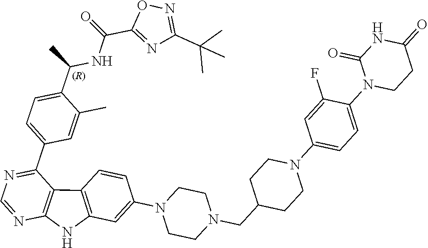

(R)-3-(tert-Butyl)-N-(1-(2-methyl-4-(7-(piperazin-1-yl)-9H-pyrimido[4,5-b]indol-4-yl)phenyl)ethyl)-1,2,4-oxadiazole-5-carboxamide (100 mg, 0.19 mmol), 1-(4-(2,4-dioxotetrahydropyrimidin-1(2H)-yl)-3-fluorophenyl)piperidine-4-carbaldehyde (160 mg, 0.50 mmol) and AcOH (0.02 mL) was dissolved in MeOH/DCM (1:10, 5 mL). The mixture was stirred at room temperature for 1 h, and NaBH(OAc)₃ (201.0 mg, 0.95 mmol) was added to the solution in portions. The resulting mixture was stirred at room temperature for another 1 h until LC-MS indicated all the starting material was consumed. The mixture was concentrated and purified with SiO₂-gel column (DCM:MeOH=10:1) to give the titled product (27.7 mg, 17.3%). ¹H NMR (400 MHz, DMSO) δ 12.25 (s, 1H), 10.45 (s, 1H), 10.01 (d, J=7.7 Hz, 1H), 8.90 (s, 1H), 7.92-7.69 (m, 4H), 7.26 (t, J=8.9 Hz, 1H), 7.00 (d, J=7.3 Hz, 2H), 6.86 (dd, J=19.1, 11.6 Hz, 2H), 5.61-5.36 (m, 1H), 3.83 (d, J=11.8 Hz, 2H), 3.70 (t, J=6.4 Hz, 2H), 3.42 (s, 2H), 3.33 (s, 4H), 2.87-2.72 (m, 5H), 2.62 (s, 3H), 2.31 (s, 2H), 1.86 (t, J=11.4 Hz, 3H), 1.64 (t, J=12.0 Hz, 3H), 1.45 (s, 9H), 1.34-1.20 (m, 3H); [M+H]⁺=842.6.

### Example 10 and 11: (R)-3-(tert-butyl)-N-(1-(4-(7-(1-((1-(4-(2,4-dioxotetrahydropyrimidin-1(2H)-yl)phenyl)piperidin-4-yl)methyl)-2,5-dihydro-1H-pyrrol-3-yl)-9H-pyrimido[4,5-b]indol-4-yl)-2-methylphenyl)ethyl)-1,2,4-oxadiazole-5-carboxamide and (R)-3-(tert-butyl)-N-(1-(4-(7-(1-((1-(4-(2,4-dioxotetrahydropyrimidin-1(2H)-yl)phenyl)piperidin-4-yl)methyl)-1H-pyrrol-3-yl)-9H-pyrimido[4,5-b]indol-4-yl)-2-methylphenyl)ethyl)-1,2,4-oxadiazole-5-carboxamide

### Step 1: ethyl 2-(4-bromo-2-nitrophenyl)-2-cyanoacetate

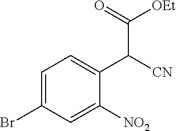 [SMILES: CCOC(=O)C(C#N)c1ccc(Br)cc1[N+](=O)[O-]]

To a stirred mixture of NaH (10.15 g, 253.753 mmol, 60%) in DMF (200 mL) was added ethyl cyanoacetate (15 mL) in DMF (150 mL) dropwise at 0° C. under nitrogen atmosphere. The resulting mixture was stirred for 0.5 h at 0° C. under nitrogen atmosphere. To the above mixture was added 4-bromo-1-chloro-2-nitrobenzene (15 g, 63.438 mmol) at 0° C. The resulting mixture was stirred for additional 4 h at 70° C. The mixture was allowed to cool down to room temperature and acidified to pH 6 with HCl (aq.) (1M). The resulting mixture was extracted with EtOAc (3×300 mL). The combined organic layers were washed with brine (3×100 mL) and dried over anhydrous Na₂SO₄. After filtration, the filtrate was concentrated under reduced pressure. The residue was purified by silica gel column chromatography, eluted with Pet. ether/EtOAc (1:1) to afford the product (14.5 g, 73%). [M−H]⁻=310.9.

### Step 2: ethyl 2-amino-6-bromo-1H-indole-3-carboxylate

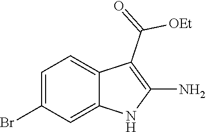 [SMILES: CCOC(=O)c1c(N)[nH]c2cc(Br)ccc12]

A mixture of ethyl 2-(4-bromo-2-nitrophenyl)-2-cyanoacetate (14 g, 44.713 mmol), Fe (24.97 g, 447.131 mmol) and NH₄Cl (2.39 g, 44.713 mmol) in EtOH (140 mL) was stirred for 2 h at 80° C. The mixture was allowed to cool down to room temperature. The resulting mixture was filtered and the filter cake was washed with MeOH (2×100 mL). The filtrate was concentrated under reduced pressure. The resulting mixture was extracted with EtOAc (3×200 mL). The combined organic layers were washed with brine (2×100 mL) and dried over anhydrous Na₂SO₄. After filtration, the filtrate was concentrated under reduced pressure. The residue was purified by silica gel column chromatography, eluted with Pet. ether/EtOAc (1:1) to afford the product (6.7 g, 52%). [M+H]⁺=283.0.

### Step 3: 7-bromo-3,9-dihydro-4H-pyrimido[4,5-b]indol-4-one

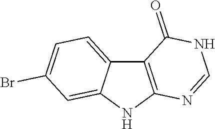 [SMILES: O=c1[nH]cnc2[nH]c3cc(Br)ccc3c12]

A mixture of ethyl 2-amino-6-bromo-1H-indole-3-carboxylate (3 g, 10.596 mmol) and formamidine acetate (4.41 g, 42.384 mmol) in 2-methoxyethanol (30 mL) was stirred for 24 h at 125° C. The mixture was allowed to cool down to room temperature. To the resulting mixture was added water and filtered. The filter cake was washed with water (3×20 mL). The solid was dried under infrared light to afford the product (2.5 g, 85%). [M+H]⁺=264.0.

### Step 4: tert-butyl 3-(4-oxo-4,9-dihydro-3H-pyrimido[4,5-b]indol-7-yl)-2,5-dihydro-1H-pyrrole-1-carboxylate

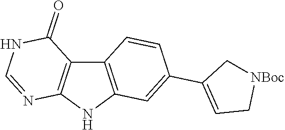 [SMILES: CC(C)(C)OC(=O)N1CC=C(c2ccc3c(c2)[nH]c2nc[nH]c(=O)c23)C1]

To a stirred mixture of 7-bromo-3,9-dihydro-4H-pyrimido[4,5-b]indol-4-one (6.1 g, 23.099 mmol) and tert-butyl 3-(4,4,5,5-tetramethyl-1,3,2-dioxaborolan-2-yl)-2,5-dihydropyrrole-1-carboxylate (8.9 g, 30.015 mmol) in dioxane (45 mL) and H₂O (9 mL) were added Pd(dppf)Cl₂ (1.7 g, 2.310 mmol) and K₃PO₄ (9.8 g, 46.198 mmol) at room temperature under nitrogen atmosphere. The resulting mixture was stirred for 16 h at 80° C. under nitrogen atmosphere. The desired product could be detected by LCMS. The mixture was allowed to cool down to room temperature and concentrated under vacuum. The residue was purified by silica gel column chromatography, eluted with CH₂Cl₂/MeOH (50:1 to 5:1) to afford the product (4.8 g, 59%). [M+H]⁺=353.1.

### Step 5: 4-chloro-7-(2,5-dihydro-1H-pyrrol-3-yl)-9H-pyrimido[4,5-b]indole

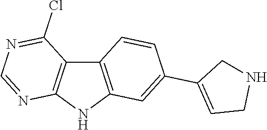 [SMILES: Clc1ncnc2[nH]c3cc(C4=CCNC4)ccc3c12]

A mixture of tert-butyl 3-(4-oxo-4,9-dihydro-3H-pyrimido[4,5-b]indol-7-yl)-2,5-dihydro-1H-pyrrole-1-carboxylate (4.8 g, 13.621 mmol) in POCl₃ (75 mL) was stirred for 16 h at 110° C. The desired product could be detected by LCMS. The resulting mixture was concentrated under vacuum to afford the product (3.7 g, crude). [M+H]⁺=271.1.

### Step 6: tert-butyl 3-(4-chloro-9H-pyrimido[4,5-b]indol-7-yl)-2,5-dihydro-1H-pyrrole-1-carboxylate

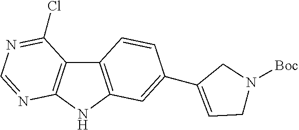 [SMILES: CC(C)(C)OC(=O)N1CC=C(c2ccc3c(c2)[nH]c2ncnc(Cl)c23)C1]

The crude 4-chloro-7-(2,5-dihydro-1H-pyrrol-3-yl)-9H-pyrimido[4,5-b]indole (3.6 g, crude) was neutralized to pH 7 with saturated NaHCO₃ (aq.). To the above mixture were added DMF (80 mL), Boc₂O (29 g, 132.979 mmol) and DMAP (0.16 g, 1.330 mmol) at room temperature. The resulting mixture was stirred for 16 h at room temperature. The desired product could be detected by LCMS. The resulting mixture was concentrated under vacuum to afford the product (2.9 g, 59%). [M+H]⁺=371.1.

### Step 7: tert-butyl 7-(1-(tert-butoxycarbonyl)-2,5-dihydro-1H-pyrrol-3-yl)-4-chloro-9H-pyrimido[4,5-b]indole-9-carboxylate

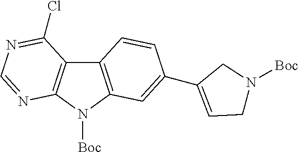 [SMILES: CC(C)(C)OC(=O)N1CC=C(c2ccc3c4c(Cl)ncnc4n(C(=O)OC(C)(C)C)c3c2)C1]

To a stirred mixture of tert-butyl 3-(4-chloro-9H-pyrimido[4,5-b]indol-7-yl)-2,5-dihydro-1H-pyrrole-1-carboxylate (2.9 g, 7.820 mmol) and DIEA (4 g, 31.280 mmol) in DMF (60 mL) were added DMAP (0.10 g, 0.782 mmol) and Boc₂O (8.5 g, 39.084 mmol) in portions at room temperature. The resulting mixture was stirred for 16 h at room temperature. The desired product could be detected by LCMS. The resulting mixture was extracted with EtOAc. The combined organic layers were washed with brine and dried over anhydrous Na₂SO₄. After filtration, the filtrate was concentrated under reduced pressure. The residue was purified by silica gel column chromatography, eluted with CH₂Cl₂/EtOAc (25:1 to 5:1) to afford the product (2.2 g, 61%). ¹H NMR (400 MHz, CDCl₃) δ 8.97 (s, 1H), 8.38 (d, J=8.3 Hz, 2H), 7.62 (s, 1H), 6.35 (s, 1H), 4.62 (s, 2H), 4.41 (d, J=15.9 Hz, 2H), 1.82 (s, 9H), 1.55 (s, 9H); [M+H]⁺=471.1.

### Step 8: tert-butyl (R)-7-(1-(tert-butoxycarbonyl)-2,5-dihydro-1H-pyrrol-3-yl)-4-(4-(1-(3-(tert-butyl)-1,2,4-oxadiazole-5-carboxamido)ethyl)-3-methylphenyl)-9H-pyrimido[4,5-b]indole-9-carboxylate

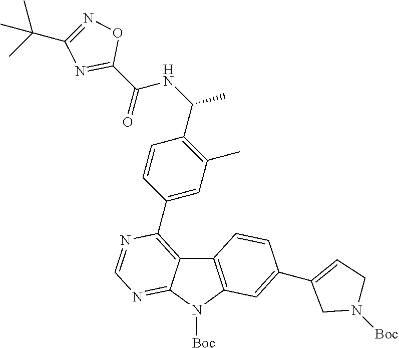 [SMILES: Cc1cc(-c2ncnc3c2c2ccc(C4=CCN(C(=O)OC(C)(C)C)C4)cc2n3C(=O)OC(C)(C)C)ccc1[C@@H](C)NC(=O)c1nc(C(C)(C)C)no1]

A mixture of tert-butyl 7-(1-(tert-butoxycarbonyl)-2,5-dihydro-1H-pyrrol-3-yl)-4-chloro-9H-pyrimido[4,5-b]indole-9-carboxylate (200 mg, 0.425 mmol), (R)-3-(tert-butyl)-N-(1-(2-methyl-4-(4,4,5,5-tetramethyl-1,3,2-dioxaborolan-2-yl)phenyl)ethyl)-1,2,4-oxadiazole-5-carboxamide (174 mg, 0.425 mmol), Pd(dppf)Cl₂ (15.5 mg, 0.021 mmol) and K₂CO₃ (95 mg, 0.68 mmol) in 1,4-dioxane (10 mL) and H₂O (2 mL) was stirred in a round bottom flask at 90° C. overnight. The mixture was concentrated in vacuum to afford the crude product, which was further purified with silica gel column chromatography (PE:EA=100%: 0%˜0%: 100%) to give the product (174 mg, 56%). [M+H]⁺=722.0.

### Step 9: (R)-3-(tert-butyl)-N-(1-(4-(7-(2,5-dihydro-1H-pyrrol-3-yl)-9H-pyrimido[4,5-b]indol-4-yl)-2-methylphenyl)ethyl)-1,2,4-oxadiazole-5-carboxamide hydrochloride

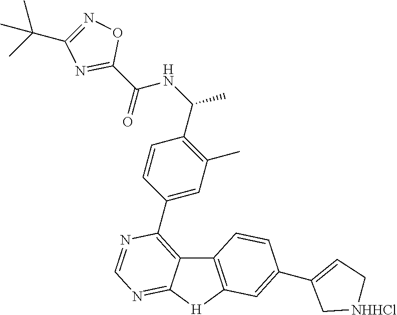

A solution of tert-butyl (R)-7-(1-(tert-butoxycarbonyl)-2,5-dihydro-1H-pyrrol-3-yl)-4-(4-(1-(3-(tert-butyl)-1,2,4-oxadiazole-5-carboxamido)ethyl)-3-methylphenyl)-9H-pyrimido[4,5-b]indole-9-carboxylate (174 mg, 0.241 mmol) in 4 M HCl in dioxane (10 mL) was stirred in a round bottom flask at room temperature for 2 hours. The mixture was then stirred at room temperature overnight. The mixture was evaporated in vacuum to afford the product (220 mg, crude), which was used for next step without further purification.

### Step 10: (R)-3-(tert-butyl)-N-(1-(4-(7-(1-((1-(4-(2,4-dioxotetrahydropyrimidin-1(2H)-yl)phenyl)piperidin-4-yl)methyl)-2,5-dihydro-1H-pyrrol-3-yl)-9H-pyrimido[4,5-b]indol-4-yl)-2-methylphenyl)ethyl)-1,2,4-oxadiazole-5-carboxamide and (R)-3-(tert-butyl)-N-(1-(4-(7-(1-((1-(4-(2,4-dioxotetrahydropyrimidin-1(2H)-yl)phenyl)piperidin-4-yl)methyl)-1H-pyrrol-3-yl)-9H-pyrimido[4,5-b]indol-4-yl)-2-methylphenyl)ethyl)-1,2,4-oxadiazole-5-carboxamide

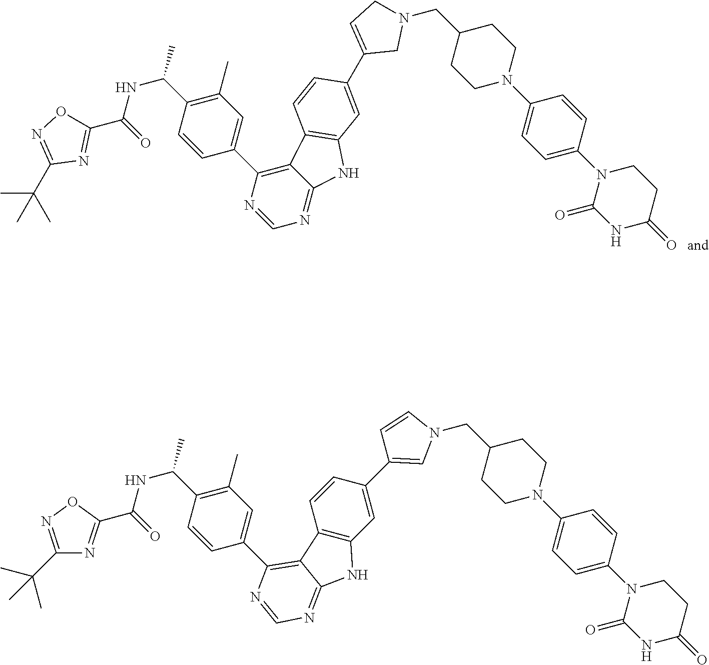 [SMILES: Cc1cc(-c2ncnc3[nH]c4cc(C5=CCN(CC6CCN(c7ccc(N8CCC(=O)NC8=O)cc7)CC6)C5)ccc4c23)ccc1[C@@H](C)NC(=O)c1nc(C(C)(C)C)no1 ; "and" ; Cc1cc(-c2ncnc3[nH]c4cc(-c5ccn(CC6CCN(c7ccc(N8CCC(=O)NC8=O)cc7)CC6)c5)ccc4c23)ccc1[C@@H](C)NC(=O)c1nc(C(C)(C)C)no1]

To a solution of (R)-3-(tert-butyl)-N-(1-(4-(7-(2,5-dihydro-1H-pyrrol-3-yl)-9H-pyrimido[4,5-b]indol-4-yl)-2-methylphenyl)ethyl)-1,2,4-oxadiazole-5-carboxamide hydrochloride (220 mg, crude) in dichloromethane (6 mL) and MeOH (6 mL), NaOAc (104 mg, 1.269 mmol) was added. The mixture was stirred at room temperature for 5 mins. Then 1-(4-(2,4-dioxotetrahydropyrimidin-1(2H)-yl)phenyl)piperidine-4-carbaldehyde (191 mg, 0.635 mmol) and HOAc (0.06 mL) were added. The mixture was stirred at room temperature overnight. Then NaBH(OAc)₃ (447 mg, 2.11 mmol) was added and stirred at room temperature for 2 hours. Then the mixture was concentrated in vacuum, and the residue was purified with silica gel column chromatography (DCM:MeOH=100%:0%˜90%:10% gradient elution) to give the crude product, which was further purified by prep-HPLC to afford Example 10 (9.45 mg, 2.7%) and Example 11 (3.4 mg, 1%). Example 10: ¹H NMR (500 MHz, DMSO) δ 12.49 (s, 1H), 10.27 (s, 1H), 9.97 (d, J=5 Hz, 1H), 8.95 (s, 1H), 7.83-7.68 (m, 4H), 7.48 (s, 1H), 7.40 (d, J=10 Hz, 1H), 7.14 (d, J=10 Hz, 2H), 6.94 (d, J=10 Hz, 2H), 6.44 (s, 1H), 5.49-5.39 (m, 1H), 3.87 (s, 2H), 3.75-3.66 (m, 4H), 3.64 (s, 2H), 2.72-2.63 (m, 4H), 2.58 (d, J=5.0 Hz, 2H), 2.53-2.51 (m, 3H), 1.87 (d, J=10 Hz, 2H), 1.59 (d, J=5.0 Hz, 3H), 1.37 (s, 9H), 1.30-1.21 (m, 2H); [M+H]⁺=807.7. Example 11: ¹H NMR (500 MHz, DMSO) δ 12.37 (s, 1H), 10.27 (s, 1H), 9.97 (d, J=5.0 Hz, 1H), 8.90 (s, 1H), 7.85-7.70 (m, 4H), 7.61 (s, 1H), 7.41 (dd, J=10.0, 1.5 Hz, 1H), 7.37-7.32 (m, 1H), 7.12 (d, J=10.0 Hz, 2H), 6.92 (d, J=10.0 Hz, 2H), 6.85 (t, J=2.5 Hz, 1H), 6.49 (d, J=2.0 Hz, 1H), 5.48-5.40 (m, 1H), 3.85 (d, J=10.0 Hz, 2H), 3.77-3.63 (m, 4H), 2.70-2.60 (m, 4H), 2.55-2.50 (m, 4H), 1.91 (s, 1H), 1.59 (d, J=10.0 Hz, 5H), 1.38 (s, 9H), 1.34-1.28 (m, 2H); [M+H]⁺=805.6.

### Example 12: 3-(tert-butyl)-N-((1R)-1-(4-(7-(1-((1-(4-(2,4-dioxotetrahydropyrimidin-1(2H)-yl)phenyl)piperidin-4-yl)methyl)pyrrolidin-3-yl)-9H-pyrimido[4,5-b]indol-4-yl)-2-methylphenyl)ethyl)-1,2,4-oxadiazole-5-carboxamide

### Step 1: tert-butyl 3-(4-oxo-4,9-dihydro-3H-pyrimido[4,5-b]indol-7-yl)-2,5-dihydro-1H-pyrrole-1-carboxylate

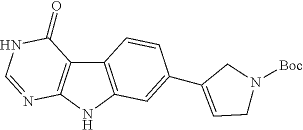 [SMILES: CC(C)(C)OC(=O)N1CC=C(c2ccc3c(c2)[nH]c2nc[nH]c(=O)c23)C1]

To a stirred mixture of 7-bromo-3,9-dihydro-4H-pyrimido[4,5-b]indol-4-one (6.1 g, 23.1 mmol) and tert-butyl 3-(4,4,5,5-tetramethyl-1,3,2-dioxaborolan-2-yl)-2,5-dihydropyrrole-1-carboxylate (8.9 g, 30.0 mmol) in dioxane (45 mL) and H₂O (9 mL) were added Pd(dppf)Cl₂ (1.7 g, 2.31 mmol) and K₃PO₄ (9.8 g, 46.20 mmol) at room temperature under nitrogen atmosphere. The resulting mixture was stirred for 16 h at 80° C. under nitrogen atmosphere. Desired product could be detected by LCMS. The mixture was allowed to cool down to room temperature. The resulting mixture was concentrated under vacuum. The residue was purified by silica gel column chromatography, eluted with CH₂Cl₂/MeOH (50:1 to 5:1) to afford the product (4.8 g, 59%). [M+H]⁺=353.1.

### Step 2: tert-butyl 3-(4-oxo-4,9-dihydro-3H-pyrimido[4,5-b]indol-7-yl)pyrrolidine-1-carboxylate

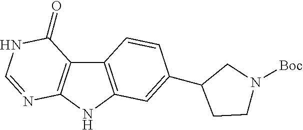 [SMILES: CC(C)(C)OC(=O)N1CCC(c2ccc3c(c2)[nH]c2nc[nH]c(=O)c23)C1]

To a stirred solution of tert-butyl 3-(4-oxo-4,9-dihydro-3H-pyrimido[4,5-b]indol-7-yl)-2,5-dihydro-1H-pyrrole-1-carboxylate (7 g, 19.8 mmol) in MeOH (140 mL), EtOAc (80 mL) and DCM (80 mL) were added Pd/C (2.1 g, 19.8 mmol) and Pd(OH)₂/C (2.79 g, 19.9 mmol). The resulting mixture was stirred for 7 days at 50° C. under hydrogen atmosphere in 20 atm. The mixture was allowed to cool down to rt. The resulting mixture was filtered and the filter cake was washed with MeOH (3×50 mL). The filtrate was concentrated under reduced pressure to afford the product (5.3 g, 75%). [M+H]⁺=355.2.

### Step 3: 4-chloro-7-(pyrrolidin-3-yl)-9H-pyrimido[4,5-b]indole

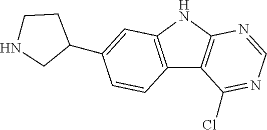 [SMILES: Clc1ncnc2[nH]c3cc(C4CCNC4)ccc3c12]

A mixture of tert-butyl 3-(4-oxo-4,9-dihydro-3H-pyrimido[4,5-b]indol-7-yl)pyrrolidine-1-carboxylate (5.1 g, 14.39 mmol) in POCl₃ (100 mL) was stirred for 2 h at 110° C. The resulting mixture was concentrated under vacuum and diluted with toluene. The resulting mixture was concentrated under vacuum to afford the crude product (7.4 g, 98%), which was used in the next step directly without further purification. [M+H]⁺=273.0.

### Step 4: tert-butyl 3-(4-chloro-9H-pyrimido[4,5-b]indol-7-yl)pyrrolidine-1-carboxylate

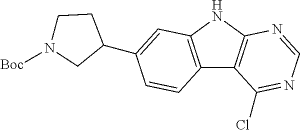 [SMILES: CC(C)(C)OC(=O)N1CCC(c2ccc3c(c2)[nH]c2ncnc(Cl)c23)C1]

The crude 4-chloro-7-(pyrrolidin-3-yl)-9H-pyrimido[4,5-b]indole (7.4 g, 27.132 mmol) was basified by saturated NaHCO₃ (aq.) to pH=8. Then DMF (75 mL), Boc₂O (59.2 g, 271.3 mmol) and DMAP (0.33 g, 2.70 mmol) were added at room temperature. The resulting mixture was stirred for additional 16 h at room temperature. The resulting mixture was diluted with water (500 mL) and extracted with EtOAc (3×200 mL). The combined organic layers were washed with brine (2×100 mL) and dried over anhydrous Na₂SO₄. After filtration, the filtrate was concentrated under reduced pressure to afford the product (3 g, 29.66%), which was used in the next step directly without further purification. [M+H]⁺=373.0.

### Step 5: tert-butyl 7-(1-(tert-butoxycarbonyl)pyrrolidin-3-yl)-4-chloro-9H-pyrimido[4,5-b]indole-9-carboxylate

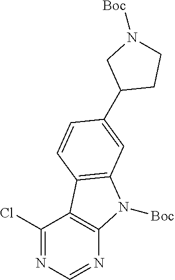 [SMILES: CC(C)(C)OC(=O)N1CCC(c2ccc3c4c(Cl)ncnc4n(C(=O)OC(C)(C)C)c3c2)C1]

A solution of tert-butyl 3-(4-chloro-9H-pyrimido[4,5-b]indol-7-yl)pyrrolidine-1-carboxylate (3 g, 11.0 mmo) and in DCM (100 mL) was stirred for 5 min at room temperature. To the above mixture was added Boc₂O (14.4 g, 65.98 mmol) and DMAP (4.5 mg, 0.037 mmol) at room temperature. The resulting mixture was stirred for additional 16 h. The resulting mixture was concentrated under reduced pressure. The residue was purified by silica gel column chromatography, eluted with PE/EtOAc (2:1) to afford the product (2 g, 38%). [M+H]⁺=473.2.

### Step 6: tert-butyl 7-(1-(tert-butoxycarbonyl)pyrrolidin-3-yl)-4-(4-((R)-1-(3-(tert-butyl)-1,2,4-oxadiazole-5-carboxamido)ethyl)-3-methylphenyl)-9H-pyrimido[4,5-b]indole-9-carboxylate

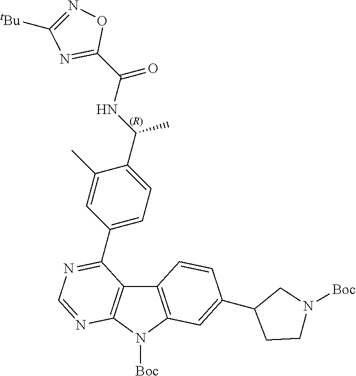

To a stirred solution of tert-butyl 7-(1-(tert-butoxycarbonyl)pyrrolidin-3-yl)-4-chloro-9H-pyrimido[4,5-b]indole-9-carboxylate (2 g, 4.23 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (2.1 g, 5.08 mmol) in dioxane (20 mL) and H₂O (4 mL) were added K₃PO₄ (1.8 g, 8.48 mmol) and Pd(dppf)Cl₂ (0.3 g, 0.42 mmol) in portions at room temperature. The resulting mixture was stirred for 2 h at 80° C. under nitrogen atmosphere and concentrated under vacuum. The residue was purified by silica gel column chromatography, eluted with PE/EtOAc (15:1 to 3:1) to afford the product (1.2 g, 40%). [M+H]⁺=724.3.

### Step 7: 3-(tert-butyl)-N-((1R)-1-(2-methyl-4-(7-(pyrrolidin-3-yl)-9H-pyrimido[4,5-b]indol-4-yl)phenyl)ethyl)-1,2,4-oxadiazole-5-carboxamide formate

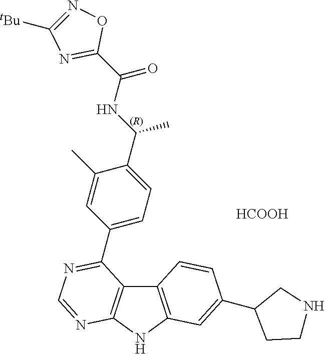

A solution of tert-butyl 7-(1-(tert-butoxycarbonyl)pyrrolidin-3-yl)-4-(4-((R)-1-(3-(tert-butyl)-1,2,4-oxadiazole-5-carboxamido)ethyl)-3-methylphenyl)-9H-pyrimido[4,5-b]indole-9-carboxylate (1.1 g, 1.50 mmol) and TFA (3 mL, 40.39 mmol) in DCM (9 mL) was stirred for 1 h at room temperature. The resulting mixture was concentrated under vacuum. The residue was purified by reverse phase flash chromatography with the following conditions: Column: Spherical C18, 20-40 um, 330 g; Mobile Phase A: Water (0.5% FA); Mobile Phase B: ACN; Flow rate: 80 mL/min; Gradient: 5%-5% B, 10 min, 50% B-95% B gradient in 30 min; Detector: 220 nm. The fractions containing the desired product were collected at 85% B and concentrated under reduced pressure to afford the product (390.3 mg, 46%). [M+H]⁺=524.3.

### Step 8: 3-(tert-butyl)-N-((1R)-1-(4-(7-(1-((1-(4-(2,4-dioxotetrahydropyrimidin-1(2H)-yl)phenyl)piperidin-4-yl)methyl)pyrrolidin-3-yl)-9H-pyrimido[4,5-b]indol-4-yl)-2-methylphenyl)ethyl)-1,2,4-oxadiazole-5-carboxamide

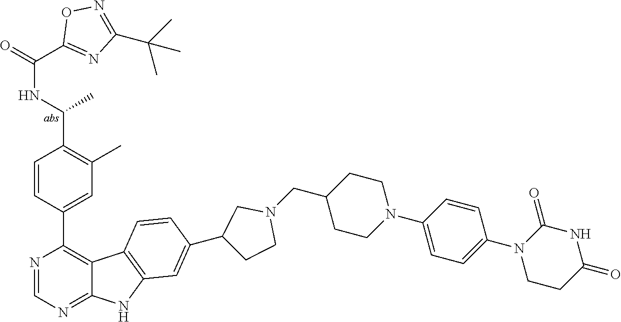 [SMILES: Cc1cc(-c2ncnc3[nH]c4cc(C5CCN(CC6CCN(c7ccc(N8CCC(=O)NC8=O)cc7)CC6)C5)ccc4c23)ccc1[C@@H](C)NC(=O)c1nc(C(C)(C)C)no1]

A mixture of 3-(tert-butyl)-N-((1R)-1-(2-methyl-4-(7-(pyrrolidin-3-yl)-9H-pyrimido[4,5-b]indol-4-yl)phenyl)ethyl)-1,2,4-oxadiazole-5-carboxamide formate (50 mg, 0.096 mmol), 1-(4-(2,4-dioxotetrahydropyrimidin-1(2H)-yl)phenyl)piperidine-4-carbaldehyde (34.5 mg, 0.115 mmol) and AcOH (0.02 mL) in MeOH (5 mL) and DCM (5 mL) was stirred at rt overnight. Then, STAB (40.5 mg, 0.19 mmol) was added to the mixture above and stirred at rt for 5 h. The mixture was diluted with water (50 mL) and extracted with DCM (3×100 mL). The combined organic layers were dried over Na₂SO₄, filtered and concentrated under vacuum. The residue was purified by silica gel column chromatography, eluted with MeOH/DCM (1:7) to afford product (51.0 mg, 66.0%). ¹H NMR (400 MHz, DMSO) δ 12.42 (s, 1H), 10.26 (s, 1H), 9.55 (d, J=4.0 Hz, 1H), 8.93 (s, 1H), 7.83-7.75 (m, 3H), 7.73-7.70 (m, 1H), 7.52 (s, 1H), 7.18 (d, J=4.0 Hz, 1H), 7.13 (d, J=8.0 Hz, 2H), 6.93 (d, J=8.0 Hz, 2H), 5.46-5.39 (m, 1H), 3.72-3.65 (m, 4H), 3.54-3.43 (m, 1H), 2.96-2.59 (m, 8H), 2.51 (s, 3H), 2.39-2.27 (m, 2H), 1.89-1.81 (m, 3H), 1.70-1.62 (m, 1H), 1.58 (d, J=4.0 Hz, 3H), 1.37 (s, 9H), 1.32-1.20 (m, 3H); [M+H]⁺=809.4.

### Example 13 and 14: 3-(tert-butyl)-N—((R)-1-(4-(7-((S)-1-((1-(4-(2,4-dioxotetrahydropyrimidin-1(2H)-yl)phenyl)piperidin-4-yl)methyl)pyrrolidin-3-yl)-9H-pyrimido[4,5-b]indol-4-yl)-2-methylphenyl)ethyl)-1,2,4-oxadiazole-5-carboxamide and 3-(tert-butyl)-N—((R)-1-(4-(7-((R)-1-((1-(4-(2,4-dioxotetrahydropyrimidin-1(2H)-yl)phenyl)piperidin-4-yl)methyl)pyrrolidin-3-yl)-9H-pyrimido[4, 5-b]indol-4-yl)-2-methylphenyl)ethyl)-1,2,4-oxadiazole-5-carboxamide

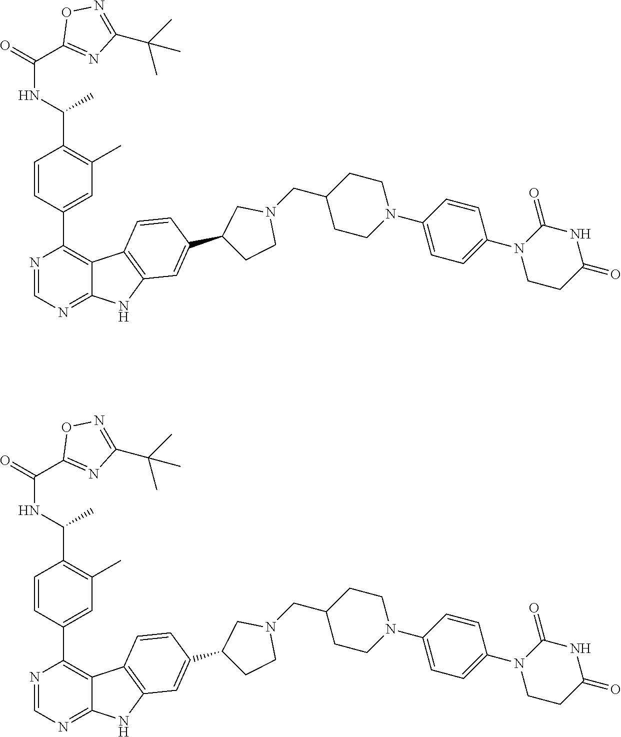 [SMILES: Cc1cc(-c2ncnc3[nH]c4cc([C@@H]5CCN(CC6CCN(c7ccc(N8CCC(=O)NC8=O)cc7)CC6)C5)ccc4c23)ccc1[C@@H](C)NC(=O)c1nc(C(C)(C)C)no1 ; Cc1cc(-c2ncnc3[nH]c4cc([C@H]5CCN(CC6CCN(c7ccc(N8CCC(=O)NC8=O)cc7)CC6)C5)ccc4c23)ccc1[C@@H](C)NC(=O)c1nc(C(C)(C)C)no1]

A racemic compound of 3-(tert-butyl)-N-((1R)-1-(4-(7-(1-((1-(4-(2,4-dioxotetrahydropyrimidin-1(2H)-yl)phenyl)piperidin-4-yl)methyl)pyrrolidin-3-yl)-9H-pyrimido[4,5-b]indol-4-yl)-2-methylphenyl)ethyl)-1,2,4-oxadiazole-5-carboxamide (48 mg) was separated by PREP_CHIRAL_HPLC with following conditions: Column: CHIRAL ART Amylose-SA, 2*25 cm, 5 um; Mobile Phase A: Hexane:DCM=3:1 (0.5% 2 M NH₃-MeOH), Mobile Phase B: IPA; Flow rate: 20 mL/min; Gradient: 25 B to 25 B in 15 min; Detector: 220/254 nm; RT1:11.095 min; RT2:13.144 min; Injection Volumn: 0.6 m1; Number Of Runs: 4; This resulted in Example 13 (15.9 mg) and Example 14 (15.5 mg). ¹H NMR of Example 13 (400 MHz, DMSO) δ 12.39 (s, 1H), 10.24 (s, 1H), 9.92 (d, J=8.0 Hz, 1H), 8.93 (s, 1H), 7.88-7.68 (m, 4H), 7.53 (s, 1H), 7.21-7.19 (m, 3H), 6.93 (d, J=8.0 Hz, 2H), 5.51-5.40 (m, 1H), 3.70 (t, J=6.7 Hz, 4H), 3.59-3.42 (m, 1H), 3.01-2.90 (m, 1H), 2.80-2.61 (m, 7H), 2.50-2.34 (m, 5H), 1.93-1.76 (m, 3H), 1.70-1.55 (m, 4H), 1.38 (s, 9H), 1.30-1.18 (m, 3H); [M+H]⁺=809.4. ¹H NMR of Example 14 (400 MHz, DMSO) δ 12.39 (s, 1H), 10.24 (s, 1H), 9.92 (d, J=8.0 Hz, 1H), 8.93 (s, 1H), 7.87-7.68 (m, 3H), 7.53 (s, 1H), 7.21-7.10 (m, 3H), 6.93 (d, J=8.6 Hz, 2H), 5.51-5.40 (m, 1H), 3.70 (t, J=6.7 Hz, 3H), 3.51-3.40 (m, 1H), 2.99-2.90 (m, 1H), 2.80-2.60 (m, 10H), 2.35 (s, 3H), 1.93-1.76 (m, 3H), 1.77-1.60 (m, 4H), 1.38 (s, 9H), 1.32-1.17 (m, 3H); [M+H]⁺=809.4.

### Example 15: 3-(tert-butyl)-N-((1R)-1-(4-(7-(4-(3-(4-(2,6-dioxopiperidin-3-yl)phenyl)cyclobutyl)piperazin-1-yl)-9H-pyrimido[4,5-b]indol-4-yl)-2-methylphenyl)ethyl)-1,2,4-oxadiazole-5-carboxamide

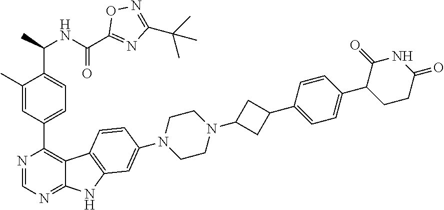 [SMILES: Cc1cc(-c2ncnc3[nH]c4cc(N5CCN(C6CC(c7ccc(C8CCC(=O)NC8=O)cc7)C6)CC5)ccc4c23)ccc1[C@@H](C)NC(=O)c1nc(C(C)(C)C)no1]

To a solution of (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide hydrobromide (0.4 g, 0.6 mmol) and 3-(4-(3-oxocyclobutyl)phenyl)piperidine-2,6-dione (154 mg, 0.6 mmol) in DCM/EtOH (30 mL) was added NaOAc (148 mg, 1.8 mmol). After stirring for 1 hour, NaBH(OAc)₃ (381 mg, 1.8 mmol) was added. The reaction mixture was stirred for another 18 hours. The solvent was evaporated and purified by pre-TLC with DCM/MeOH (10:1) to give the product (33.0 mg, 7.1%). ¹H NMR (400 MHz, DMSO) δ 12.18 (s, 1H), 10.82 (s, 1H), 9.94 (d, J=8.0 Hz, 1H), 8.82 (s, 1H), 7.85-7.65 (m, 4H), 7.21-7.14 (m, 4H), 6.94 (s, 2H), 5.41 (t, J=7.6 Hz, 1H), 3.82 (dd, J=4.8 Hz, 11.2 Hz, 1H), 3.26-3.15 (m, 5H), 2.76-2.63 (m, 3H), 2.50 (brs, 10H), 2.25-2.16 (m, 1H), 2.03 (brs, 1H), 1.91 (brs, 1H), 1.58 (d, J=6.8 Hz, 3H), 1.37 (s, 9H); [M+H]⁺=780.7.

### Example 16: 3-(tert-butyl)-N-((1R)-1-(4-(7-(4-(2-(5-(2,6-dioxopiperidin-3-yl)-6-methylpyridin-2-yl)ethyl)piperazin-1-yl)-9H-pyrimido[4,5-b]indol-4-yl)-2-methylphenyl)ethyl)-1,2,4-oxadiazole-5-carboxamide

### Step 1: diethyl 2-(5-bromo-6-methylpyridin-2-yl)malonate

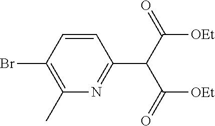 [SMILES: CCOC(=O)C(C(=O)OCC)c1ccc(Br)c(C)n1]

A mixture of 3,6-dibromo-2-methylpyridine (10 g, 40 mmol), diethyl malonate (12.8 g, 80 mmol), CuI (1.53 g, 8 mmol), 2-picolinic acid (1.97 g, 16 mmol) and Cs₂CO₃ (52.17 g, 160 mmol) in 1,4-dioxane (100 mL) was stirred in a round bottom flask at 100° C. for 16 hours. The reaction was quenched by addition of brine and extracted with DCM. The DCM layer was evaporated in vacuum and purified with silica gel column chromatography to give the product (18.69 g, crude).

### Step 2: 2-(5-bromo-6-methylpyridin-2-yl)acetic acid

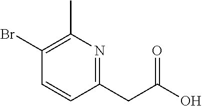

To a solution of diethyl 2-(5-bromo-6-methylpyridin-2-yl)malonate (18.69 g, 57 mmol) in MeOH/H₂O (80 mL/40 mL) was added NaOH (6.37 g, 160 mmol) at 25° C. The mixture was stirred at 25° C. for 3 h, then concentrated in vacuo. The residue was dissolved in water and then adjusted to pH=3˜4 with 2 N HCl/H₂O. The solution was purified by reverse phase chromatography to give the product (12 g, 92%).

### Step 3: 2-(5-bromo-6-methylpyridin-2-yl)ethan-1-ol

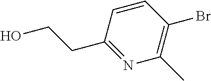

To a solution of 2-(5-bromo-6-methylpyridin-2-yl)acetic acid (11 g, 57 mmol) in THF (200 mL) was added BH₃·THF (1.0 M, 70 mL) at 0° C. The mixture was stirred at 25° C. for 16 h. The reaction was quenched by addition of MeOH (70 mL), then concentrated in vacuo. The residue was dissolved in sat. NaHCO₃ and extracted with DCM. The DCM layer was evaporated in vacuum to afford the crude product, which was further purified with silica gel column chromatography to give the product (4.9 g, 47%). [M+H]⁺=216.1.

### Step 4: 2-(2′,6′-bis(benzyloxy)-2-methyl-[3,3′-bipyridin]-6-yl)ethan-1-ol

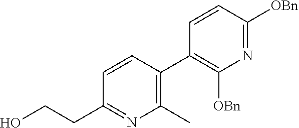 [SMILES: Cc1nc(CCO)ccc1-c1ccc(OCc2ccccc2)nc1OCc1ccccc1]

A mixture of 2-(5-bromo-6-methylpyridin-2-yl)ethan-1-ol (4.9 g, 22.7 mmol), 2,6-bis(benzyloxy)-3-(4,4,5,5-tetramethyl-1,3-dioxolan-2-yl)pyridine (9 g, 21.5 mmol), Pd(PPh₃)Cl₂ (0.5 g, 0.71 mmol) and Na₂CO₃ (5 g, 47.2 mmol) in 1,4-dioxane (80 mL) and H₂O (20 mL) was stirred in a round bottom flask at 100° C. overnight. The mixture was evaporated in vacuum to afford the crude product, which was further purified with silica gel column chromatography to give the product (11 g, crude). [M+H]⁺=427.4.

### Step 5: 3-(6-(2-hydroxyethyl)-2-methylpyridin-3-yl)piperidine-2,6-dione

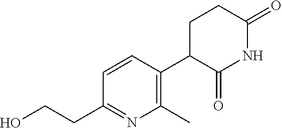 [SMILES: Cc1nc(CCO)ccc1C1CCC(=O)NC1=O]

Under H₂ (4 atm), a mixture of 2-(2′,6′-bis(benzyloxy)-2-methyl-[3,3′-bipyridin]-6-yl)ethan-1-ol (8 g, 18.8 mmol) and 10% Pd/C (2.6 g) in MeOH (70.0 mL) was stirred for 16 hours at 40° C. The mixture was filtered through a pad of Celite and washed with MeOH (40.0 mL). The filtrate was concentrated under vacuum to obtain the product (3.4 g, 73%). [M+H]⁺=249.1.

### Step 6: 2-(5-(2,6-dioxopiperidin-3-yl)-6-methylpyridin-2-yl)ethyl methanesulfonate

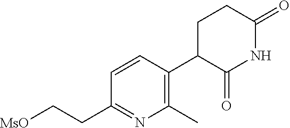 [SMILES: Cc1nc(CCOS(C)(=O)=O)ccc1C1CCC(=O)NC1=O]

To a solution of 3-(6-(2-hydroxyethyl)-2-methylpyridin-3-yl)piperidine-2,6-dione (240 mg, 0.97 mmol) and Et₃N (1 mL, 7.22 mmol) in DCM (20 mL) was added MsCl (420 mg, 3.66 mmol). The mixture was allowed to stir at 25° C. for 2 hours. The mixture was evaporated in vacuum to afford the crude product, which was further purified with silica gel column chromatography to give the product (170 mg, 54%). [M+H]⁺=327.3.

### Step 7: 3-(tert-butyl)-N-((1R)-1-(4-(7-(4-(2-(5-(2,6-dioxopiperidin-3-yl)-6-methylpyridin-2-yl)ethyl)piperazin-1-yl)-9H-pyrimido[4,5-b]indol-4-yl)-2-methylphenyl)ethyl)-1,2,4-oxadiazole-5-carboxamide

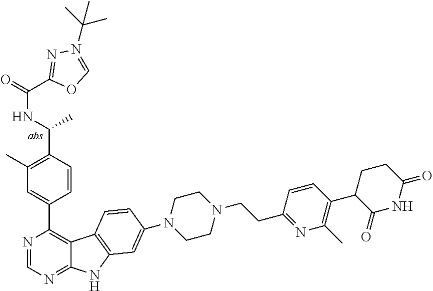

A mixture of (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide (50 mg, 0.093 mmol), 2-(5-(2,6-dioxopiperidin-3-yl)-6-methylpyridin-2-yl)ethyl methanesulfonate (80 mg, 0.245 mmol), KI (300 mg, 1.8 mmol) and DIPEA (100 mg, 0.78 mmol) in MeCN (20 mL) was stirred in a round bottom flask under reflux for 16 hours. Then the mixture was evaporated in vacuum to afford the crude product, which was purified with silica gel column chromatography (DCM:MeOH=100:0˜100:10) to give the product (10 mg, 14%). ¹H NMR (400 MHz, DMSO) δ 12.18 (s, 1H), 10.87 (s, 1H), 9.90-9.97 (m, 1H), 8.80 (s, 1H), 7.63-7.80 (m, 4H), 7.43 (s, 1H), 7.11 (s, 1H), 6.91 (s, 2H), 5.40 (s, 1H), 3.99-4.09 (m, 1H), 3.18-3.28 (m, 6H), 2.87 (s, 2H), 2.57-2.76 (m, 6H), 2.41 (s, 3H), 2.13-2.34 (m, 3H), 1.88-2.00 (m, 2H), 1.52-1.59 (m, 3H), 1.35 (s, 9H); [M+H]⁺=769.6.

### Example 17: (R)-3-(tert-butyl)-N-(1-(4-(7-(4-(4-(2,4-dioxotetrahydropyrimidin-1(2H)-yl)-3-methylphenethyl)piperazin-1-yl)-9H-pyrimido[4,5-b]indol-4-yl)-2-methylphenyl)ethyl)-1,2,4-oxadiazole-5-carboxamide

### Step 1: 2-(4-(2,6-dioxopiperidin-3-yl)phenyl)acetaldehyde

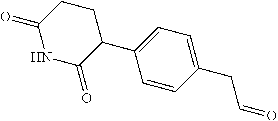 [SMILES: O=CCc1ccc(C2CCC(=O)NC2=O)cc1]

A mixture of 3-(4-(2-hydroxyethyl)phenyl)piperidine-2,6-dione (200 mg, 0.8 mmol) and IBX (451 mg, 1.6 mmol) in DMSO (5 mL) was stirred in a round bottom flask at room temperature overnight. The reaction was quenched with water and the mixture was extracted with EtOAc and washed with saturated aqueous NaCl and twice with saturated aqueous NaHCO₃. The organic layer was dried over anhydrous Na₂SO₄ and evaporated in vacuum to afford the product (150 mg, 75%). [M+H]⁺=232.3.

### Step 2: (R)-3-(tert-butyl)-N-(1-(4-(7-(4-(4-(2,4-dioxotetrahydropyrimidin-1(2H)-yl)-3-methylphenethyl)piperazin-1-yl)-9H-pyrimido[4,5-b]indol-4-yl)-2-methylphenyl)ethyl)-1,2,4-oxadiazole-5-carboxamide

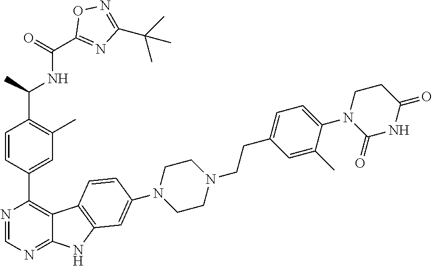 [SMILES: Cc1cc(-c2ncnc3[nH]c4cc(N5CCN(CCc6ccc(N7CCC(=O)NC7=O)c(C)c6)CC5)ccc4c23)ccc1[C@@H](C)NC(=O)c1nc(C(C)(C)C)no1]

A mixture of (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide hydrobromide (200 mg, 0.33 mmol) and 2-(4-(2,6-dioxopiperidin-3-yl)phenyl)acetaldehyde (90 mg, 0.364 mmol) in MeOH (10 mL) was stirred in a round bottom flask for 30 min at room temperature. Then NaBH(OAc)₃ (140 mg, 0.66 mmol) was added and stirred for 2 h at room temperature. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (50 mg, 19.7%). ¹H NMR (400 MHz, DMSO) δ 12.20 (s, 1H), 10.33 (s, 1H), 9.95 (s, 1H), 8.83 (s, 1H), 7.78-7.65 (m, 4H), 7.30-7.10 (m, 3H), 7.05-6.90 (m, 2H), 5.42 (s, 1H), 3.78-3.70 (m, 1H), 3.56-3.40 (m, 2H), 3.30-3.20 (m, 5H), 2.80-2.60 (m, 9H), 2.16 (s, 3H), 1.60-1.50 (m, 3H), 1.37 (s, 9H); [M+H]⁺=769.5.

### Example 18: 3-(tert-butyl)-N-((1R)-1-(4-(7-(4-(4-(2,6-dioxopiperidin-3-yl)-3-methylphenethyl)piperazin-1-yl)-9H-pyrimido[4,5-b]indol-4-yl)-2-methylphenyl)ethyl)-1,2,4-oxadiazole-5-carboxamide

### Step 1: 2-(4-(2,6-dioxopiperidin-3-yl)-3-methylphenyl)acetaldehyde

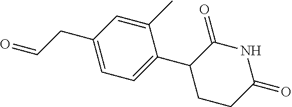 [SMILES: Cc1cc(CC=O)ccc1C1CCC(=O)NC1=O]

3-(4-(2-Hydroxyethyl)-2-methylphenyl)piperidine-2,6-dione (300 mg, 1.2 mmol) was placed in DMSO (5 mL), and IBX (672 mg, 2.4 mmol) was added to the solution in portions at room temperature. The resulting mixture was stirred at room temperature for 16 h until TLC indicated all the starting material was consumed. The mixture was diluted with water (25 mL) and extracted with EtOAc (25 mL*3). The combined organic layer was washed with brine (50 mL*2), dried over Na₂SO₄ and concentrated to provide the desired product (460 mg, crude) which was used directly without further purification. [M+H]⁺=246.2.

### Step 2: 3-(tert-butyl)-N-((1R)-1-(4-(7-(4-(4-(2,6-dioxopiperidin-3-yl)-3-methylphenethyl)piperazin-1-yl)-9H-pyrimido[4,5-b]indol-4-yl)-2-methylphenyl)ethyl)-1,2,4-oxadiazole-5-carboxamide

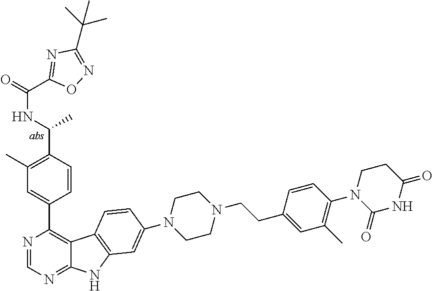 [SMILES: Cc1cc(-c2ncnc3[nH]c4cc(N5CCN(CCc6ccc(N7CCC(=O)NC7=O)c(C)c6)CC5)ccc4c23)ccc1[C@@H](C)NC(=O)c1nc(C(C)(C)C)no1]

(R)-3-(tert-Butyl)-N-(1-(2-methyl-4-(7-(piperazin-1-yl)-9H-pyrimido[4,5-b]indol-4-yl)phenyl)ethyl)-1,2,4-oxadiazole-5-carboxamide (100 mg, 0.19 mmol), 2-(4-(2,6-dioxopiperidin-3-yl)-3-methylphenyl)acetaldehyde (460 mg, crude) and AcOH (0.02 mL) was dissolved in MeOH/DCM (1:10, 5 mL). The mixture was stirred at room temperature for 1 h and NaBH(OAc)₃ (201.0 mg, 0.95 mmol) was added to the solution in portions. The resulting mixture was stirred at room temperature for another 1 h until LC-MS indicated all the starting material was consumed. The mixture was concentrated and purified with SiO₂-gel column (DCM:MeOH=10:1) to give the titled product (63.3 mg, 43.4%). ¹H NMR (400 MHz, DMSO) δ 12.18 (s, 1H), 10.83 (s, 1H), 9.95 (d, J=7.3 Hz, 1H), 8.83 (s, 1H), 7.78 (s, 1H), 7.70 (d, J=7.7 Hz, 3H), 7.15-7.08 (m, 2H), 6.94 (s, 3H), 5.42 (s, 1H), 4.00 (d, J=7.8 Hz, 2H), 3.26 (s, 4H), 3.00 (s, 2H), 2.73 (s, 4H), 2.62 (s, 4H), 2.49-2.48 (m, 3H), 2.25 (s, 3H), 2.15 (s, 1H), 1.98-1.94 (m, 2H), 1.58 (d, J=5.9 Hz, 3H), 1.37 (s, 9H); [M+H]⁺=768.6.

### Example 19: (R)-3-(tert-butyl)-N-(1-(4-(7-(4-(2-(5-(2,4-dioxotetrahydropyrimidin-1(2H)-yl)pyridin-2-yl)ethyl)piperazin-1-yl)-9H-pyrimido[4,5-b]indol-4-yl)-2-methylphenyl)ethyl)-1,2,4-oxadiazole-5-carboxamide

### Step 1: 2-(5-bromopyridin-2-yl)ethan-1-ol

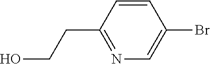

To a solution of 2-(5-bromopyridin-2-yl)acetic acid (4.3 g, 20 mmol) in THF (20 mL) was added BH₃/THF (1 N, 60 mL) at 0-10° C. The reaction mixture was stirred at 20-30° C. for 18 hours. After quenching the reaction with MeOH, HCl (2 mL) and HOAc (5 mL) was added to mixture and stirred for 30 min. The solvent was evaporated. To the residue was added EA and washed with NaHCO₃ (50 mL) solution and brine (50 mL). The organic phase was separated, concentrated and used for next step directly.

### Step 2: 5-bromo-2-(2-((tert-butyldimethylsilyl)oxy)ethyl)pyridine

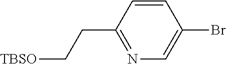 [SMILES: CC(C)(C)[Si](C)(C)OCCc1ccc(Br)cn1]

To a solution of 2-(5-bromopyridin-2-yl)ethan-1-ol (6.0 g, 30 mmol) in DMF (50 mL) was added imidazole (6.1 g, 90 mmol) and TBSCl (5.4 g, 36 mmol). The reaction mixture was stirred at 20-30° C. for 18 hours. After quenching the reaction with H₂O, the reaction mixture was extracted with EA. The organic phase was washed with brine (100 mL*2), concentrated and purified by chromatography with PE/EA (100:1 to 5:1) to give the product (8 g, 84.4%). ¹H NMR (400 MHz, CDCl₃) δ 8.63 (s, 1H), 7.75 (d, J=8.0 Hz, 1H), 7.17 (d, J=8.0 Hz, 1H), 4.00 (t, J=6.0 Hz, 2H), 2.99 (t, J=6.0 Hz, 2H), 0.90 (s, 9H), 0.02 (s, 6H).

### Step 3: (6-(2-((tert-butyldimethylsilyl)oxy)ethyl)pyridin-3-yl)boronic acid

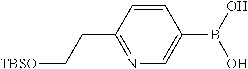 [SMILES: CC(C)(C)[Si](C)(C)OCCc1ccc(B(O)O)cn1]

To a solution of 5-bromo-2-(2-((tert-butyldimethylsilyl)oxy)ethyl)pyridine (1.0 g, 3.2 mmol) and triisopropyl borate (0.77 g, 4.1 mmol) in THF (20 mL) was added BuLi (3 mL, 4.8 mmol) dropwise at −60-−70° C. After stirring for 10 min at 20-30° C., the reaction was quenched with NH₄Cl solution (30 mL). The organic phase was evaporated and extracted with EA (30 mL). The organic phase was separated and washed with brine (30 mL). The mixture was concentrated and used for next step directly.

### Step 4: 1-(6-(2-hydroxyethyl)pyridin-3-yl)-3-((2-(trimethylsilyl)ethoxy)methyl)pyrimidine-2,4(1H,3H)-dione

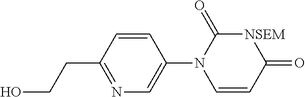 [SMILES: C[Si](C)(C)CCOCn1c(=O)ccn(-c2ccc(CCO)nc2)c1=O]

A solution of (6-(2-((tert-butyldimethylsilyl)oxy)ethyl)pyridin-3-yl)boronic acid (6.0 g, 21.3 mmol), Cu(OAc)₂ (7.7 g, 42.3 mmol), molecular sieve (6.0 g), pyridine (8.5 g, 107.6 mmol) and 3-((2-(trimethylsilyl)ethoxy)methyl)pyrimidine-2,4(1H,3H)-dione (0.77 g, 21.5 mmol) in DMA (50 mL) was stirred at 80° C. for 2 hours under 02 (1 atm). The reaction was quenched with H₂O (100 mL) and extracted with EA (100 mL). The solid was filtrated off. The organic phase was separated and washed with brine (200 mL*3). The solvent was evaporated and purified by chromatography with PE/EA (100:1 to 1:1) followed by DCM/MeOH (100:1 to 10:1). The crude product was used for next step. [M+H]⁺=364.2.

### Step 5: 1-(6-(2-hydroxyethyl)pyridin-3-yl)-3-((2-(trimethylsilyl)ethoxy)methyl)dihydropyrimidine-2,4(1H,3H)-dione

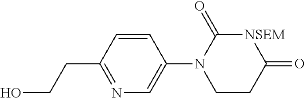 [SMILES: C[Si](C)(C)CCOCN1C(=O)CCN(c2ccc(CCO)nc2)C1=O]

A solution of 1-(6-(2-hydroxyethyl)pyridin-3-yl)-3-((2-(trimethylsilyl)ethoxy)methyl)pyrimidine-2,4(1H,3H)-dione (1.0 g, 2.7 mmol) and Pd/C (0.1 g) in MeOH was stirred at room temperature for 18 hours under H₂ (1 atm). After the solid was filtered off, the filtrate was concentrated and used for next step directly.

### Step 6: 2-(5-(2,4-dioxotetrahydropyrimidin-1(2H)-yl)pyridin-2-yl)ethyl methanesulfonate

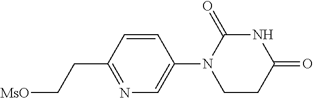 [SMILES: CS(=O)(=O)OCCc1ccc(N2CCC(=O)NC2=O)cn1]

A solution of 1-(6-(2-hydroxyethyl)pyridin-3-yl)-3-((2-(trimethylsilyl)ethoxy)methyl)dihydropyrimidine-2,4(1H,3H)-dione (0.2 g, 0.5 mmol), MsCl (0.17 g, 1.5 mmol) and DIEA (0.32 g, 2.5 mmol) in DCM was stirred at room temperature for 3 hours. The solvent was evaporated and used for next step directly.

### Step 7: (R)-3-(tert-butyl)-N-(1-(4-(7-(4-(2-(5-(2,4-dioxotetrahydropyrimidin-1(2H)-yl)pyridin-2-yl)ethyl)piperazin-1-yl)-9H-pyrimido[4,5-b]indol-4-yl)-2-methylphenyl)ethyl)-1,2,4-oxadiazole-5-carboxamide

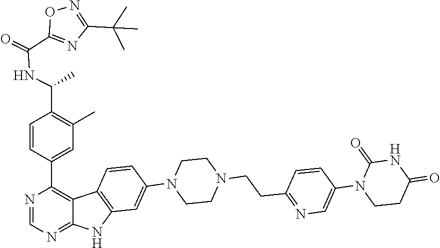 [SMILES: Cc1cc(-c2ncnc3[nH]c4cc(N5CCN(CCc6ccc(N7CCC(=O)NC7=O)cn6)CC5)ccc4c23)ccc1[C@@H](C)NC(=O)c1nc(C(C)(C)C)no1]

A solution of 2-(5-(2,4-dioxotetrahydropyrimidin-1(2H)-yl)pyridin-2-yl)ethyl methanesulfonate (80 mg, 0.18 mmol), (R)-3-(tert-butyl)-N-(1-(2-methyl-4-(7-(piperazin-1-yl)-9H-pyrimido[4,5-b]indol-4-yl)phenyl)ethyl)-1,2,4-oxadiazole-5-carboxamide (100 mg, 0.18 mmol), DIEA (93 mg, 0.7 mmol) and KI (179 mg, 1.1 mmol) in ACN (30 mL) was stirred at 80-85° C. for 18 hours. The solvent was evaporated. The residue was diluted with H₂O (30 mL) and extracted with EA (30 mL*2). The organic phase was combined and purified by pre-TLC with PE/EA. The product was dissolved in DCM (5 mL). A solution of HCl in dioxane (4 N, 20 mL) was added. The mixture was stirred for 1 hour. After evaporating the solvent, the residue was dissolved in MeOH (10 mL). A solution of NH₄₀H (4 drops) was added and the mixture was stirred for 30 min. The solvent was evaporated and purified by pre-TLC with DCM/MeOH˜10:1 to give the product (21.7 mg, 16%). ¹H NMR (400 MHz, DMSO) δ 12.19 (s, 1H), 10.47 (s, 1H), 9.93 (d, J=8.0 Hz, 1H), 8.82 (s, 1H), 8.48 (s, 1H), 7.91-7.67 (m, 5H), 7.37 (d, J=8.0 Hz, 1H), 6.94 (s, 2H), 5.42 (t, J=7.2 Hz, 1H), 3.81 (t, J=6.8 Hz, 2H), 3.26 (s, 4H), 2.96 (s, 2H), 2.73 (t, J=6.4 Hz, 4H), 2.64 (s, 4H), 2.50 (s, 3H), 1.58 (d, J=6.8 Hz, 3H), 1.37 (s, 9H); [M+H]⁺=756.7.

### Example 20: 3-(tert-butyl)-N-((1R)-1-(4-(7-(1-(3-(4-((R)-2,6-dioxopiperidin-3-yl)phenyl)cyclobutyl)pyrrolidin-3-yl)-9H-pyrimido[4,5-b]indol-4-yl)-2-methylphenyl)ethyl)-1,2,4-oxadiazole-5-carboxamide

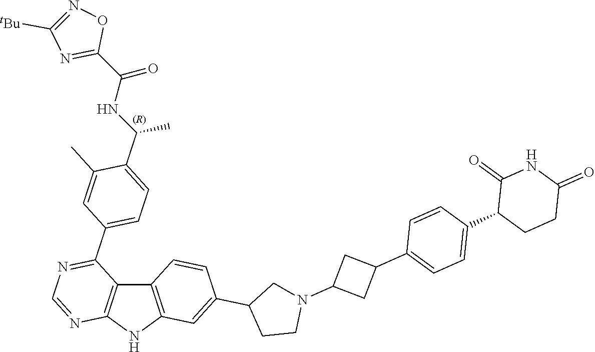

A mixture of 3-(tert-butyl)-N-((1R)-1-(2-methyl-4-(7-(pyrrolidin-3-yl)-9H-pyrimido[4,5-b]indol-4-yl)phenyl)ethyl)-1,2,4-oxadiazole-5-carboxamide (60 mg, 0.115 mmol) and (R)-3-(4-(3-oxocyclobutyl)phenyl)piperidine-2,6-dione (35.4 mg, 0.137 mmol) in DCE (10 mL) was stirred at 80° C. for 16 h under a nitrogen atmosphere. Then, NaBH₃CN (14.5 mg, 0.23 mmol) was added to the mixture above at rt. The mixture was stirred at rt for 5 h. Then the mixture was diluted with water (50 mL) and extracted with DCM (3×100 mL). The combined organic layers were dried over Na₂SO₄, filtered and concentrated under vacuum. The residue was purified by Prep-TLC (DCM/MeOH=9:1) to afford the product (13.07 mg, 14.9%). ¹H NMR (500 MHz, DMSO) δ 12.41 (s, 1H), 10.83 (s, 1H), 9.94 (d, J=5.0 Hz, 1H), 8.93 (s, 1H), 7.86-7.69 (m, 4H), 7.56-7.50 (m, 1H), 7.30-7.12 (m, 5H), 5.47-5.37 (m, 1H), 3.87-3.79 (m, 1H), 3.67-3.47 (m, 2H), 3.23-3.05 (m, 3H), 2.95-2.74 (m, 3H), 2.72-2.60 (m, 3H), 2.52 (s, 3H), 2.40-2.33 (m, 1H), 2.28-2.13 (m, 2H), 2.08-1.99 (m, 2H), 1.95-1.82 (m, 1H), 1.58 (d, J=5.0 Hz, 3H), 1.37 (s, 9H); [M+H]⁺=765.4.

### Example 21: 3-(tert-butyl)-N-((1R)-1-(4-(7-(1-(3-(4-((S)-2,6-dioxopiperidin-3-yl)phenyl)cyclobutyl)pyrrolidin-3-yl)-9H-pyrimido[4,5-b]indol-4-yl)-2-methylphenyl)ethyl)-1,2,4-oxadiazole-5-carboxamide

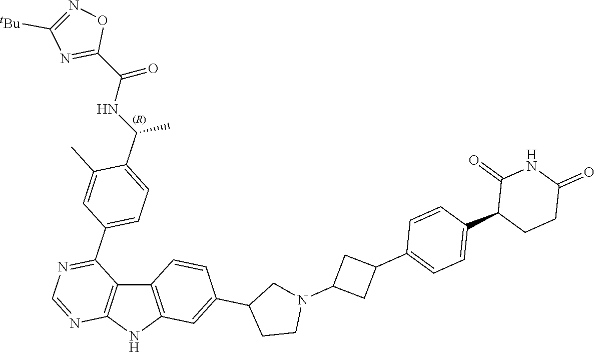

The titled compound was synthesized in the procedures similar to Example 20. ¹H NMR (500 MHz, DMSO) δ 12.51 (s, 1H), 10.84 (s, 1H), 9.96 (d, J=10.0 Hz, 1H), 8.95 (s, 1H), 7.86-7.70 (m, 4H), 7.54 (s, 1H), 7.31-7.16 (m, 5H), 5.47-5.38 (m, 1H), 3.90-3.78 (m, 2H), 3.68-3.56 (m, 2H), 3.32-3.18 (m, 4H), 2.74-2.56 (m, 6H), 2.52 (s, 3H), 2.24-2.14 (m, 2H), 2.07-1.98 (m, 2H), 1.58 (d, J=5.0 Hz, 3H), 1.37 (s, 9H); [M+H]⁺=765.4.

### Example 22: (R)-5-(tert-butyl)-N-(1-(4-(7-(4-((1-(4-(2,4-dioxotetrahydropyrimidin-1(2H)-yl)-3-methylphenyl)piperidin-4-yl)methyl)piperazin-1-yl)-9H-pyrimido[4,5-b]indol-4-yl)-2-methylphenyl)ethyl)-1,2,4-oxadiazole-3-carboxamide

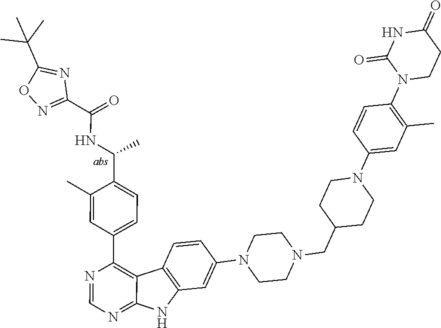 [SMILES: Cc1cc(-c2ncnc3[nH]c4cc(N5CCN(CC6CCN(c7ccc(N8CCC(=O)NC8=O)c(C)c7)CC6)CC5)ccc4c23)ccc1[C@@H](C)NC(=O)c1noc(C(C)(C)C)n1]

A mixture of (R)-5-(tert-butyl)-N-(1-(2-methyl-4-(7-(piperazin-1-yl)-9H-pyrimido[4,5-b]indol-4-yl)phenyl)ethyl)-1,2,4-oxadiazole-3-carboxamide hydrobromide (the compound was obtained through the similar way in example 5) (100 mg, 0.161 mmol) and 1-(4-(2,4-dioxotetrahydropyrimidin-1(2H)-yl)-3-methylphenyl)piperidine-4-carbaldehyde (43.7 mg, 0.178 mmol) in MeOH (10 mL) was stirred in a round bottom flask for 30 min at room temperature. Then NaBH(OAc)₃ (68.3 mg, 0.322 mmol) was added and stirred for 2 h at room temperature. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (20 mg, 14.8%). ¹H NMR (400 MHz, DMSO) δ 12.17 (s, 1H), 10.24 (s, 1H), 9.58-9.48 (m, 1H), 7.91-7.59 (m, 4H), 7.09-6.73 (m, 5H), 5.48-5.40 (m, 1H), 3.80-3.60 (m, 3H), 3.50-3.40 (m, 1H), 3.28-3.20 (m, 4H), 2.80-2.60 (m, 4H), 2.28-2.20 (m, 2H), 2.12 (s, 3H), 1.95-1.65 (m, 3H), 1.56 (d, J=6.8 Hz, 3H), 1.43 (s, 9H), 1.30-1.15 (m, 3H); [M+H]⁺=838.8.

### Example 23: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-5-fluoro-2-methylphenyl)ethyl)-1,2,4-oxadiazole-5-carboxamide

### Step 1: benzyl 4-(4-chloro-3-nitrophenyl)piperazine-1-carboxylate

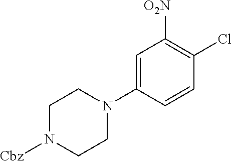 [SMILES: O=C(OCc1ccccc1)N1CCN(c2ccc(Cl)c([N+](=O)[O-])c2)CC1]

A mixture of 1-chloro-4-fluoro-2-nitrobenzene (30 g, 170.901 mmol), benzyl piperazine-1-carboxylate (45 g, 205.081 mmol) and K₂CO₃ (35 g, 256.352 mmol) in DMSO (600 mL) was stirred at 100° C. for 16 h. The mixture was allowed to cool down to room temperature. The resulting mixture was diluted with EtOAc (1000 mL) and washed with water (3×500 mL). The organic layer was washed with brine (300 mL) and dried over anhydrous Na₂SO₄. After filtration, the filtrate was concentrated under reduced pressure. The residue was purified by silica gel column chromatography, eluted with EtOAc in Pet. ether (10%-30%) to afford the product (10 g, 16%). [M+H]⁺=376.1.

### Step 2: benzyl 4-(4-(1-cyano-2-ethoxy-2-oxoethyl)-3-nitrophenyl)piperazine-1-carboxylate

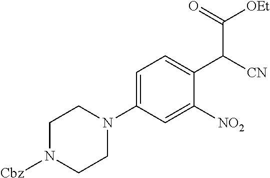 [SMILES: CCOC(=O)C(C#N)c1ccc(N2CCN(C(=O)OCc3ccccc3)CC2)cc1[N+](=O)[O-]]

To a stirred mixture of Cs₂CO₃ (33 g, 101.115 mmol) in DMF (100 mL) was added ethyl cyanoacetate (8 mL, 71.342 mmol) in DMF (50 mL) dropwise at 0° C. under nitrogen atmosphere. The resulting mixture was stirred for 0.5 h at room temperature under nitrogen atmosphere. To the above mixture was added benzyl 4-(4-chloro-3-nitrophenyl)piperazine-1-carboxylate (9.5 g, 25.279 mmol) at 0° C. The resulting mixture was stirred for additional 16 h at 70° C. The mixture was allowed to cool down to room temperature. The mixture was acidified to pH 6 with HCl (1 M). The resulting mixture was extracted with EtOAc (3×300 mL). The combined organic layers were washed with brine (100 mL) and dried over anhydrous Na₂SO₄. After filtration, the filtrate was concentrated under reduced pressure. The residue was purified by silica gel column chromatography, eluted with PE/EtOAc (1:1) to afford the product (8.4 g, 73%). [M+H]⁺=453.2.

### Step 3: ethyl 2-amino-6-(4-((benzyloxy)carbonyl)piperazin-1-yl)-1H-indole-3-carboxylate

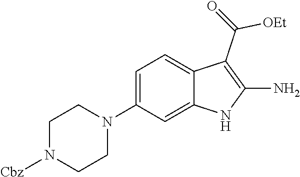 [SMILES: CCOC(=O)c1c(N)[nH]c2cc(N3CCN(C(=O)OCc4ccccc4)CC3)ccc12]

To a stirred mixture of benzyl 4-(4-(1-cyano-2-ethoxy-2-oxoethyl)-3-nitrophenyl)piperazine-1-carboxylate (8.5 g, 18.786 mmol) in EtOH (85 mL) were added Fe (8.4 g, 150.287 mmol) and NH₄Cl (2 g, 37.572 mmol) at room temperature. The resulting mixture was stirred for 16 h at 80° C. The resulting mixture was filtered and the filter cake was washed with DCM/CH₃OH (10:1, 200 mL). The filtrate was concentrated under reduced pressure. The residue was purified by silica gel column chromatography, eluted with PE/EtOAc (1:5) to afford the product (4.2 g, 53%). [M+H]⁺=423.2.

### Step 4: benzyl 4-(4-oxo-4,9-dihydro-3H-pyrimido[4,5-b]indol-7-yl)piperazine-1-carboxylate

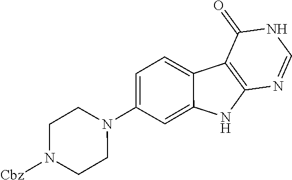 [SMILES: O=C(OCc1ccccc1)N1CCN(c2ccc3c(c2)[nH]c2nc[nH]c(=O)c23)CC1]

To a stirred solution of ethyl 2-amino-6-(4-((benzyloxy)carbonyl)piperazin-1-yl)-1H-indole-3-carboxylate (4 g, 9.468 mmol) in 2-methoxyethan-1-ol (40 mL) was added formamidine acetate (3.9 g, 0.038 mmol) at room temperature. The resulting mixture was stirred for 16 h at 125° C. The mixture was allowed to cool down to room temperature. The reaction was quenched with water (50 mL) at room temperature. The precipitated solids were collected by filtration and washed with water (3×20 mL). The solid was dried under infrared light to afford the product (3.8 g, 99%). [M+H]⁺=404.2.

### Step 5: benzyl 4-(4-chloro-9H-pyrimido[4,5-b]indol-7-yl)piperazine-1-carboxylate

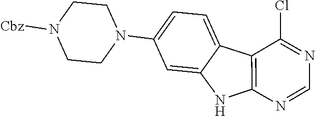 [SMILES: O=C(OCc1ccccc1)N1CCN(c2ccc3c(c2)[nH]c2ncnc(Cl)c23)CC1]

To a stirred mixture of benzyl 4-(4-oxo-4,9-dihydro-3H-pyrimido[4,5-b]indol-7-yl)piperazine-1-carboxylate (2 g, 4.957 mmol) in dioxane (20 mL) was added POCl₃ (4.6 mL, 30.136 mmol) at room temperature. The resulting mixture was stirred for 3 h at 90° C. and concentrated under reduced pressure. The resulting mixture was diluted with ethyl acetate (50 mL) and basified to pH 8 with saturated NaHCO₃ (aq., 25 mL). The resulting mixture was extracted with EtOAc (3×60 mL). The combined organic layers were washed with brine (10 mL) and dried over anhydrous Na₂SO₄. After filtration, the filtrate was concentrated under reduced pressure. The residue was purified by silica gel column chromatography, eluted with PE/EtOAc (1:1) to afford the product (1.2 g, 57%). [M+H]⁺=422.0.

### Step 6: benzyl (R)-4-(4-(4-(1-(3-(tert-butyl)-1,2,4-oxadiazole-5-carboxamido)ethyl)-2-fluoro-5-methylphenyl)-9H-pyrimido[4,5-b]indol-7-yl)piperazine-1-carboxylate

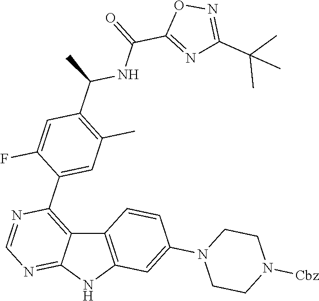 [SMILES: Cc1cc(-c2ncnc3[nH]c4cc(N5CCN(C(=O)OCc6ccccc6)CC5)ccc4c23)c(F)cc1[C@@H](C)NC(=O)c1nc(C(C)(C)C)no1]

A mixture of benzyl 4-(4-chloro-9H-pyrimido[4,5-b]indol-7-yl)piperazine-1-carboxylate (405 mg, 0.965 mmol), (R)-3-(tert-butyl)-N-(1-(5-fluoro-2-methyl-4-(4,4,5,5-tetramethyl-1,3,2-dioxaborolan-2-yl)phenyl)ethyl)-1,2,4-oxadiazole-5-carboxamide (the compound was obtained through the same way in WO2021219070A) (415 mg, 0.965 mmol), Pd(dppf)Cl₂ (80 mg, 0.097 mmol) and K₂CO₃ (555 mg, 1.74 mmol) in 1,4-dioxane (15 mL) and H₂O (3 mL) was stirred at 95° C. overnight. The mixture was evaporated in vacuum to afford the crude product, which was further purified with silica gel column chromatography (PE:EA=100%: 0%˜0%: 100% gradient elution) to give the product (290 mg, 43%). [M+H]⁺=691.4.

### Step 7: (R)-3-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-5-carboxamide

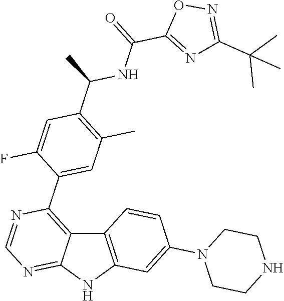 [SMILES: Cc1cc(-c2ncnc3[nH]c4cc(N5CCNCC5)ccc4c23)c(F)cc1[C@@H](C)NC(=O)c1nc(C(C)(C)C)no1]

A solution of benzyl (R)-4-(4-(4-(1-(3-(tert-butyl)-1,2,4-oxadiazole-5-carboxamido)ethyl)-2-fluoro-5-methylphenyl)-9H-pyrimido[4,5-b]indol-7-yl)piperazine-1-carboxylate (290 mg, 0.420 mmol) in trifluoroacetic acid (10 mL) was stirred at 85° C. for 2 hours. The mixture was evaporated in vacuum to afford the product (520 mg, crude), which was used in next step without further purification. [M+H]⁺=557.0.

### Step 8: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-5-fluoro-2-methylphenyl)ethyl)-1,2,4-oxadiazole-5-carboxamide

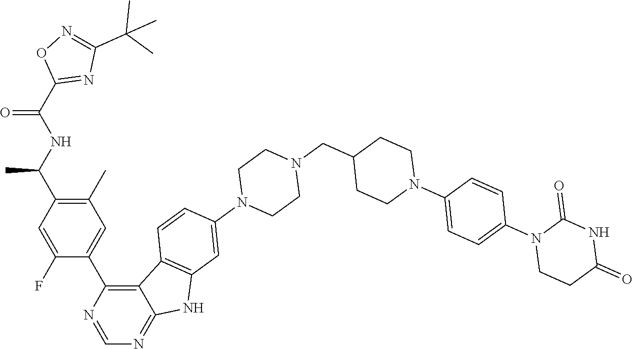 [SMILES: Cc1cc(-c2ncnc3[nH]c4cc(N5CCN(CC6CCN(c7ccc(N8CCC(=O)NC8=O)cc7)CC6)CC5)ccc4c23)c(F)cc1[C@@H](C)NC(=O)c1nc(C(C)(C)C)no1]

To a solution of (R)-3-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-5-carboxamide (120 mg, crude) in dichloromethane (4 mL) and MeOH (4 mL), NaOAc (53 mg, 0.636 mmol) was added. The mixture was stirred at room temperature for 5 mins. Then 1-(4-(2,4-dioxotetrahydropyrimidin-1(2H)-yl)phenyl)piperidine-4-carbaldehyde (the compound was obtained through the same way in WO2021219070A) (76.5 mg, 0.254 mmol) and HOAc (0.06 mL) were added. The mixture was stirred at room temperature overnight. Then NaBH(OAc)₃ (224 mg, 0.9 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (15.03 mg, 8%). ¹H NMR (500 MHz, DMSO) δ 12.21 (s, 1H), 10.25 (s, 1H), 9.53 (d, J=10.0 Hz, 1H), 8.85 (s, 1H), 7.57-7.50 (m, 2H), 7.26 (d, J=10.0 Hz, 1H), 7.13 (d, J=10.0 Hz, 2H), 7.01-6.87 (m, 4H), 5.46-5.34 (m, 1H), 3.73-3.65 (m, 4H), 3.29 (s, 1H), 3.25 (s, 4H), 2.71-2.66 (m, 4H), 2.65 (s, 1H), 2.58-2.52 (m, 5H), 2.45 (s, 3H), 2.24 (d, J=10.0 Hz, 1H), 1.82 (d, J=10.0 Hz, 2H), 1.72 (s, 1H), 1.56 (d, J=5.0 Hz, 3H), 1.44 (s, 9H); [M+H]⁺=842.6.

### Example 24: (R)-5-(tert-butyl)-N-(1-(4-(7-(4-((1-(4-(2,4-dioxotetrahydropyrimidin-1(2H)-yl)-3-methylphenyl)piperidin-4-yl)methyl)piperazin-1-yl)-9H-pyrimido[4,5-b]indol-4-yl)-5-fluoro-2-methylphenyl)ethyl)-1,2,4-oxadiazole-3-carboxamide

### Step 1: benzyl (R)-4-(4-(4-(1-(5-(tert-butyl)-1,2,4-oxadiazole-3-carboxamido)ethyl)-2-fluoro-5-methylphenyl)-9H-pyrimido[4,5-b]indol-7-yl)piperazine-1-carboxylate

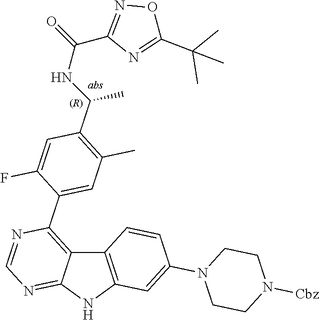

A mixture of benzyl 4-(4-chloro-9H-pyrimido[4,5-b]indol-7-yl)piperazine-1-carboxylate (400 mg, 0.95 mmol), (R)-5-(tert-butyl)-N-(1-(5-fluoro-2-methyl-4-(4,4,5,5-tetramethyl-1,3,2-dioxaborolan-2-yl)phenyl)ethyl)-1,2,4-oxadiazole-3-carboxamide (the compound was obtained through the same way in WO2021219070A) (500 mg, 1.16 mmol), Pd(dppf)Cl₂ (100 mg, 0.13 mmol) and Cs₂CO₃ (772 mg, 2.37 mmol) in 1,4-dioxane (20 mL) and water (4 mL) was stirred in a round bottom flask at 105° C. overnight. The mixture was evaporated in vacuum to afford the crude product, which was further purified with silica gel column chromatography (PE:EA=100:0˜0:100 gradient elution) to give the titled product (110 mg, 17%). [M+H]⁺=691.3.

### Step 2: (R)-5-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-3-carboxamide

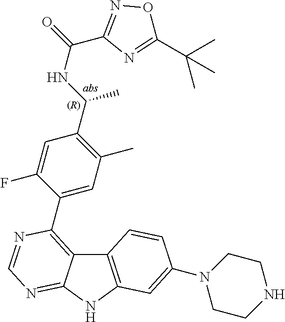

To a solution of benzyl (R)-4-(4-(4-(1-(5-(tert-butyl)-1,2,4-oxadiazole-3-carboxamido)ethyl)-2-fluoro-5-methylphenyl)-9H-pyrimido[4,5-b]indol-7-yl)piperazine-1-carboxylate (110 mg, 0.17 mmol) in MeOH (2 mL) was added 33% HBr in CH₃COOH (4 mL) at 0° C. The mixture was stirred at 25° C. for 18 hours. The mixture was evaporated in vacuum to afford the crude product, which was further purified with C18 column chromatography (0.1% FA in water:ACN=85:15˜45:55 gradient elution) to give the titled product (35 mg, 37%). [M+H]⁺=557.3.

### Step 3: (R)-5-(tert-butyl)-N-(1-(4-(7-(4-((1-(4-(2,4-dioxotetrahydropyrimidin-1(2H)-yl)-3-methylphenyl)piperidin-4-yl)methyl)piperazin-1-yl)-9H-pyrimido[4,5-b]indol-4-yl)-5-fluoro-2-methylphenyl)ethyl)-1,2,4-oxadiazole-3-carboxamide

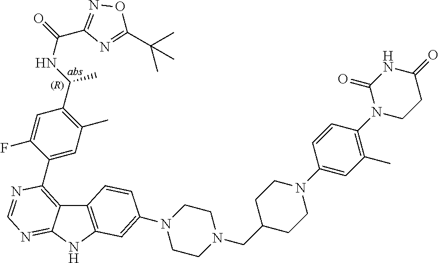

A mixture of (R)-5-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-3-carboxamide (35 mg, 0.05 mmol) and 1-(4-(2,4-dioxotetrahydropyrimidin-1(2H)-yl)-3-methylphenyl)piperidine-4-carbaldehyde (24 mg, 0.075 mmol) in dichloromethane (10 mL) and MeOH (2 mL) was stirred in a round bottom flask at room temperature for 1 hour. To the mixture was added NaBH(OAc)₃ (106 mg, 0.50 mmol) and stirred in a round bottom flask at room temperature overnight. Then the mixture was evaporated in vacuum to afford the crude product, which was purified with pre-TLC (DCM:MeOH=7:1) to give the product (26 mg, 61%). ¹H NMR (400 MHz, DMSO) δ 12.29 (s, 1H), 10.25 (s, 1H), 9.57 (d, J=7.6 Hz, 1H), 8.85 (s, 1H), 7.60-7.46 (m, 2H), 7.27 (d, J=8.4 Hz, 1H), 7.04 (d, J=8.8 Hz, 1H), 6.98-6.87 (m, 2H), 6.82 (s, 1H), 6.77 (d, J=8.4 Hz, 1H), 5.48-5.34 (m, 1H), 3.75-3.62 (m, 3H), 3.50-3.43 (m, 1H), 3.25 (s, 4H), 2.73-2.61 (m, 4H), 2.58-2.53 (m, 2H), 2.46 (s, 3H), 2.29-2.16 (m, 2H), 2.10 (s, 3H), 1.94-1.89 (m, 2H), 1.89-1.76 (m, 2H), 1.76-1.66 (m, 1H), 1.61-1.53 (m, 3H), 1.44 (s, 9H), 1.25-1.19 (m, 2H); [M+H]⁺=856.7.

### Example 25 and 26: 3-(tert-butyl)-N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-1,2,4-oxadiazole-5-carboxamide and 3-(tert-butyl)-N—((R)-1-(4-(7-(4-(3-(4-((R)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-1,2,4-oxadiazole-5-carboxamide

### Step 1: 2,6-bis(benzyloxy)-3-(4,4,5,5-tetramethyl-1,3,2-dioxaborolan-2-yl)pyridine

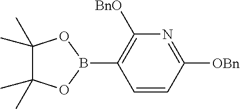 [SMILES: CC1(C)OB(c2ccc(OCc3ccccc3)nc2OCc2ccccc2)OC1(C)C]

To a solution of 2,6-bis(benzyloxy)-3-bromopyridine (40.00 g, 108.036 mmol) and 4,4,5,5-tetramethyl-2-(tetramethyl-1,3,2-dioxaborolan-2-yl)-1,3,2-dioxaborolane (41.15 g, 162.054 mmol) in dioxane (800.00 mL) were added KOAc (31.81 g, 324.109 mmol) and Pd(dppf)Cl₂ (6.32 g, 8.643 mmol). After stirring for 16 h at 100° C. under a nitrogen atmosphere, the resulting mixture was concentrated under reduced pressure. The reaction mixture was used in next step. [M+H]⁺=418.1.

### Step 2: 3-(4-(2,6-bis(benzyloxy)pyridin-3-yl)phenyl)cyclobutan-1-one

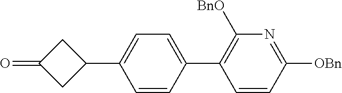 [SMILES: O=C1CC(c2ccc(-c3ccc(OCc4ccccc4)nc3OCc3ccccc3)cc2)C1]

To a solution of 3-(4-bromophenyl) cyclobutan-1-one (22.50 g, 99.962 mmol) and 2,6-bis(benzyloxy)-3-(4,4,5,5-tetramethyl-1,3,2-dioxaborolan-2-yl)pyridine (37.54 g, 89.966 mmol) in dioxane (200 mL) and water (40 mL) were added K₂CO₃ (16.58 g, 119.955 mmol) and Pd(dppf)Cl₂ (7.31 g, 9.996 mmol). After stirring for 16 h at 90° C. under a nitrogen atmosphere, the resulting mixture was concentrated under reduced pressure. The residue was purified by silica gel column chromatography, eluted with 1-20% EtOAc in PE to afford the product (30.1 g, 38.89%). [M+H]⁺=436.1.

### Step 3: 3-(4-(5,8-dioxaspiro[3.4]octan-2-yl)phenyl)-2,6-bis(benzyloxy)pyridine

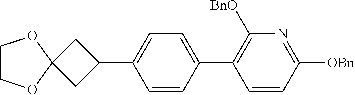 [SMILES: c1ccc(COc2ccc(-c3ccc(C4CC5(C4)OCCO5)cc3)c(OCc3ccccc3)n2)cc1]

To a solution of 3-(4-(2,6-bis(benzyloxy)pyridin-3-yl)phenyl)cyclobutan-1-one (26.00 g, 59.698 mmol) in toluene (500.00 mL) were added 4-methylbenzene-1-sulfonic acid hydrate (1.14 g, 5.970 mmol) and 2-dihydroxyethane (14.82 g, 238.793 mmol) under nitrogen atmosphere. The mixture was refluxed for 16 h through a Dean-Stark to separate the water. The resulting mixture was concentrated under reduced pressure. The residue was dissolved in EtOAc (500 mL). The combined organic layers were washed with aqueous NaHCO₃ (3×200 mL) and dried over anhydrous Na₂SO₄. After filtration, the filtrate was concentrated under reduced pressure. The residue was purified by silica gel column chromatography, eluted with 1˜40% EtOAc in PE to afford the product (25.2 g, 88.02%). [M+H]⁺=480.1.

### Step 4: 3-(4-(5,8-dioxaspiro[3.4]octan-2-yl)phenyl)piperidine-2,6-dione

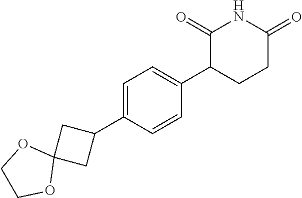 [SMILES: O=C1CCC(c2ccc(C3CC4(C3)OCCO4)cc2)C(=O)N1]

To a solution of 3-(4-(5,8-dioxaspiro[3.4]octan-2-yl)phenyl)-2,6-bis(benzyloxy)pyridine (25.00 g, 52.129 mmol) in THF (600.00 mL) and EtOH (200.00 mL) was added Pd/C (5.55 g) under nitrogen atmosphere. The mixture was stirred for 16 h under hydrogen atmosphere using a hydrogen tire. The resulting mixture was filtered through a Celite pad and the filter cake was washed with MeOH (3×20 mL). The filtrate was concentrated under reduced pressure. The resulting mixture was concentrated under reduced pressure. The residue was purified by silica gel column chromatography, eluted with 10-40% EtOAc in PE to afford the product (14.4 g, 91.67%). [M+H]⁺=302.2.

### Step 5: 3-(4-(3-oxocyclobutyl)phenyl)piperidine-2,6-dione

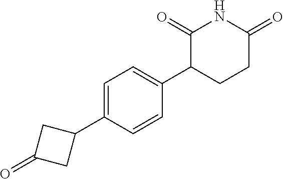 [SMILES: O=C1CC(c2ccc(C3CCC(=O)NC3=O)cc2)C1]

To a stirred solution of 3-(4-(5,8-dioxaspiro[3.4]octan-2-yl)phenyl)piperidine-2,6-dione (13.5 g, 44.8 mmol) in THF (100 mL) and water (80 mL) was added 2 M HCl (20 mL) at room temperature. The resulting mixture was stirred for 4 h at 55° C. under nitrogen atmosphere. The desired product could be detected by LCMS. The reaction was quenched with aqueous NaHCO₃ (10 mL) at 0° C. The resulting mixture was extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 10-60% EtOAc in PE to afford the product (8.2 g, 72%). [M+H]⁺=258.1.

### Step 6: (R)-3-(4-(3-oxocyclobutyl)phenyl)piperidine-2,6-dione and (S)-3-(4-(3-oxocyclobutyl)phenyl)piperidine-2,6-dione

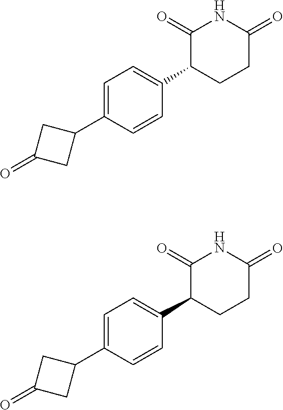 [SMILES: O=C1CC(c2ccc([C@H]3CCC(=O)NC3=O)cc2)C1 ; O=C1CC(c2ccc([C@@H]3CCC(=O)NC3=O)cc2)C1]

3-(4-(3-oxocyclobutyl)phenyl)piperidine-2,6-dione (8.20 g, 31.9 mmol) was separated by Prep-Chiral-SFC with the following conditions: Column: CHIRALPAK IH, 5*25 cm, 5 um; Mobile Phase A: CO₂, Mobile Phase B: MeOH; Flow rate: 200 mL/min; Gradient: 50% B; Column Temperature: Back Pressure: 100 bar; Detector: 220 nm; RT₁: 3.99 min; RT₂: 5.53 min; Injection Volumn: 5 mL; Number Of Runs: 30; (3R)-3-[4-(3-oxocyclobutyl)phenyl]piperidine-2,6-dione (3.30 g, 40%) was obtained at 3.99 min. ¹H NMR (400 MHz, DMSO) δ 10.84 (s, 1H), 7.36-7.32 (m, 2H), 7.21-7.19 (m, 2H), 3.86 (dd, J=11.6, 5.2 Hz, 1H), 3.84-3.83 (m, 1H), 3.47-3.40 (m, 2H), 3.34-3.18 (m, 2H), 2.70-2.62 (m, 1H), 2.52-2.47 (m, 1H), 2.20-2.17 (m, 1H), 2.05-2.01 (m, 1H); [M+H]⁺=258.1. (3S)-3-[4-(3-oxocyclobutyl)phenyl]piperidine-2,6-dione (3.23 g, 40%) was obtained at 5.53 min. ¹H NMR (400 MHz, DMSO) δ 10.84 (s, 1H), 7.38-7.31 (m, 2H), 7.23-7.13 (m, 2H), 3.85 (dd, J=11.6, 4.8 Hz, 1H), 3.71-3.58 (m, 1H), 3.50-3.37 (m, 2H), 3.28-3.21 (m, 2H), 2.70-2.62 (m, 1H), 2.52-2.47 (m, 1H), 2.22-2.18 (m, 1H), 2.05-2.01 (m, 1H); [M+H]⁺=258.1.

### Step 7: 3-(tert-butyl)-N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-1,2,4-oxadiazole-5-carboxamide

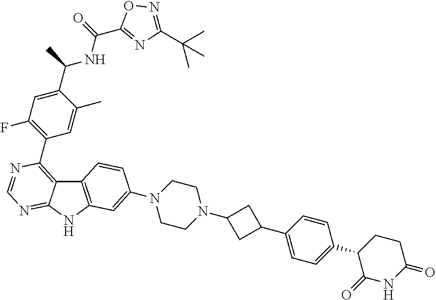 [SMILES: Cc1cc(-c2ncnc3[nH]c4cc(N5CCN(C6CC(c7ccc([C@@H]8CCC(=O)NC8=O)cc7)C6)CC5)ccc4c23)c(F)cc1[C@@H](C)NC(=O)c1nc(C(C)(C)C)no1]

To a solution of (R)-3-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-5-carboxamide (120 mg, crude) in dichloromethane (4 mL) and MeOH (4 mL), NaOAc (53 mg, 0.636 mmol) was added. The mixture was stirred at room temperature for 5 mins. Then (R)-3-(4-(3-oxocyclobutyl)phenyl)piperidine-2,6-dione (81.7 mg, 0.318 mmol) and HOAc (0.06 mL) were added. The mixture was stirred at room temperature overnight. Then NaBH(OAc)₃ (224.7 mg, 1.06 mmol) was added and stirred at room temperature overnight. Then NaBH₃CN (135 mg, 0.636 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (17.7 mg, 10%). ¹H NMR (500 MHz, DMSO) δ 12.20 (s, 1H), 10.81 (s, 1H), 9.53 (d, J=10.0 Hz, 1H), 8.86 (s, 1H), 7.56-7.50 (m, 2H), 7.31-7.24 (m, 2H), 7.23-7.19 (m, 2H), 7.18-7.12 (m, 2H), 6.93 (s, 2H), 5.46-5.35 (m, 1H), 3.85-3.77 (m, 1H), 3.30-3.21 (m, 4H), 3.15 (s, 1H), 2.75 (s, 1H), 2.70-2.60 (m, 2H), 2.49-2.42 (m, 9H), 2.23-2.12 (m, 2H), 2.06-1.98 (m, 1H), 1.94-1.84 (m, 2H), 1.56 (d, J=5.0 Hz, 3H), 1.43 (d, J=9.4 Hz, 9H); [M+H]⁺=798.6.

### Step 8: 3-(tert-butyl)-N—((R)-1-(4-(7-(4-(3-(4-((R)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-1,2,4-oxadiazole-5-carboxamide

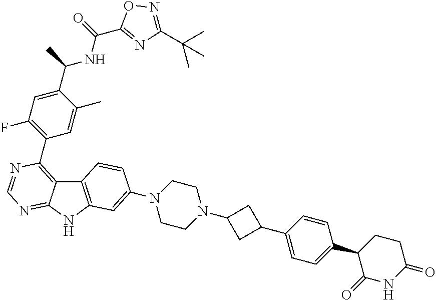 [SMILES: Cc1cc(-c2ncnc3[nH]c4cc(N5CCN(C6CC(c7ccc([C@H]8CCC(=O)NC8=O)cc7)C6)CC5)ccc4c23)c(F)cc1[C@@H](C)NC(=O)c1nc(C(C)(C)C)no1]

To a solution of (R)-3-(tert-butyl)-N-(1-(5-fluoro-2-methyl-4-(7-(piperazin-1-yl)-9H-pyrimido[4,5-b]indol-4-yl)phenyl)ethyl)-1,2,4-oxadiazole-5-carboxamide (120 mg, crude) in dichloromethane (4 mL) and MeOH (4 mL), NaOAc (53 mg, 0.636 mmol) was added. The mixture was stirred at room temperature for 5 mins. Then (S)-3-(4-(3-oxocyclobutyl)phenyl)piperidine-2,6-dione (81.7 mg, 0.318 mmol) and HOAc (0.06 mL) were added. The mixture was stirred at room temperature overnight. Then NaBH(OAc)₃ (224.7 mg, 1.06 mmol) was added and stirred at room temperature overnight. Then NaBH₃CN (135 mg, 0.636 mmol) was added and stirred at room temperature overnight. The reaction mixture was concentrated in vacuum, and the residue was purified by silica gel column chromatography (DCM:MeOH=100%: 0% 90%: 10% gradient elution) to give the crude product, which was further purified by prep-TLC (DCM:MeOH=10:1) to afford the desired product (6.75 mg, 4%). ¹H NMR (500 MHz, DMSO) δ 12.20 (s, 1H), 10.82 (s, 1H), 9.53 (d, J=10.0 Hz, 1H), 8.86 (s, 1H), 7.62-7.50 (m, 2H), 7.25-7.4 (m, 2H), 7.20-7.09 (m, 4H), 6.93 (s, 2H), 5.44-5.31 (m, 1H), 3.90-3.74 (m, 1H), 3.25 (s, 3H), 3.15 (s, 1H), 2.75 (s, 1H), 2.68-2.59 (m, 2H), 2.49-2.42 (m, 8H), 2.17 (d, J=7.7 Hz, 2H), 2.06-1.97 (m, 2H), 1.96-1.85 (m, 1H), 1.56 (d, J=7.0 Hz, 4H), 1.44 (s, 10H); [M+H]⁺=798.5.

### Example 27: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-3-fluoro-2-methylphenyl)ethyl)-1,2,4-oxadiazole-3-carboxamide

### Step 1: benzyl (R)-4-(4-(4-(1-(5-(tert-butyl)-1,2,4-oxadiazole-3-carboxamido)ethyl)-2-fluoro-3-methylphenyl)-9H-pyrimido[4,5-b]indol-7-yl)piperazine-1-carboxylate

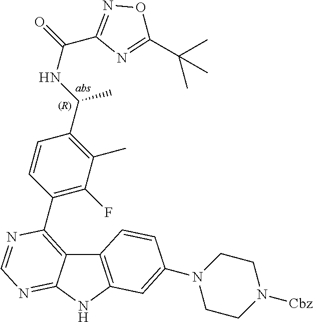

A mixture of benzyl 4-(4-chloro-9H-pyrimido[4,5-b]indol-7-yl)piperazine-1-carboxylate (420 mg, 1.0 mmol), (R)-5-(tert-butyl)-N-(1-(3-fluoro-2-methyl-4-(4,4,5,5-tetramethyl-1,3,2-dioxaborolan-2-yl)phenyl)ethyl)-1,2,4-oxadiazole-3-carboxamide (540 mg, 1.25 mmol), Pd(dppf)Cl₂ (73 mg, 0.10 mmol) and Cs₂CO₃ (1000 mg, 3.07 mmol) in 1,4-dioxane (20 mL) and water (5 mL) was stirred in a round bottom flask at 105° C. overnight. The mixture was evaporated in vacuum to afford the crude product, which was further purified with silica gel column chromatography (PE:EA=30:70˜0:100 gradient elution) to give the titled product (298 mg, 43%). [M+H]⁺=691.3.

### Step 2: (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide

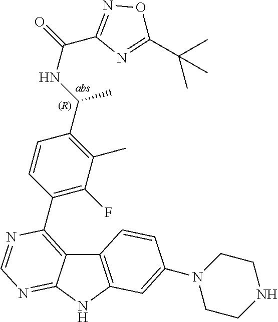

To a solution of benzyl (R)-4-(4-(4-(1-(5-(tert-butyl)-1,2,4-oxadiazole-3-carboxamido)ethyl)-2-fluoro-3-methylphenyl)-9H-pyrimido[4,5-b]indol-7-yl)piperazine-1-carboxylate (298 mg, 0.43 mmol) in MeOH (10 mL) was added 33% HBr in CH₃COOH (8 mL) at 0° C. The mixture was stirred at 25° C. for 18 h. The mixture was evaporated in vacuum to afford the crude product, which was further purified with C18 column chromatography (0.1% FA in water:ACN=75:25˜45:55 gradient elution) to give the titled product (50 mg, 21%). [M+H]⁺=557.3.

### Step 3: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-3-fluoro-2-methylphenyl)ethyl)-1,2,4-oxadiazole-3-carboxamide

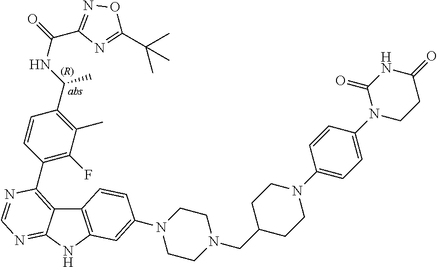

A mixture of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (25 mg, 0.045 mmol) and 1-(4-(2,4-dioxotetrahydropyrimidin-1(2H)-yl)phenyl)piperidine-4-carbaldehyde (24 mg, 0.079 mmol) in dichloromethane (10 mL) and MeOH (2 mL) was stirred in a round bottom flask at room temperature for 1 hour. To the mixture was added NaBH(OAc)₃ (106 mg, 0.50 mmol) and stirred in a round bottom flask at room temperature overnight. Then the mixture was evaporated in vacuum to afford the crude product, which was purified with pre-TLC (DCM:MeOH=9:1) to give the product (26 mg, 61%). ¹H NMR (400 MHz, DMSO) δ 12.21 (s, 1H), 10.25 (s, 1H), 9.75-9.45 (m, 1H), 8.85 (s, 1H), 7.62-7.45 (m, 2H), 7.29-7.05 (m, 3H), 6.97-6.88 (m, 4H), 5.52-5.32 (m, 1H), 3.76-3.64 (m, 5H), 3.28-3.17 (m, 4H), 2.78-2.61 (m, 5H), 2.40 (s, 4H), 2.27-2.14 (m, 2H), 1.88-1.65 (m, 4H), 1.61-1.51 (m, 3H), 1.43 (s, 9H), 1.31-1.14 (m, 3H); [M+H]⁺=842.8.

### Example 28: (R)-5-(tert-butyl)-N-(1-(4-(7-(4-((1-(4-(2,4-dioxotetrahydropyrimidin-1(2H)-yl)-3-methylphenyl)piperidin-4-yl)methyl)piperazin-1-yl)-9H-pyrimido[4,5-b]indol-4-yl)-3-fluoro-2-methylphenyl)ethyl)-1,2,4-oxadiazole-3-carboxamide

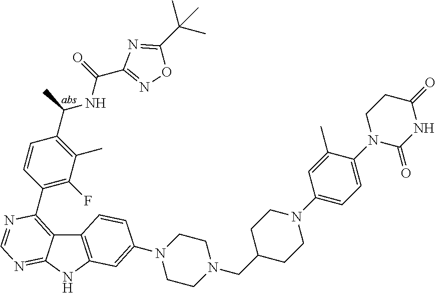 [SMILES: Cc1cc(N2CCC(CN3CCN(c4ccc5c(c4)[nH]c4ncnc(-c6ccc([C@@H](C)NC(=O)c7noc(C(C)(C)C)n7)c(C)c6F)c45)CC3)CC2)ccc1N1CCC(=O)NC1=O]

The titled compound was synthesized in the procedures similar to Example 27. ¹H NMR (400 MHz, DMSO) δ 12.26 (s, 1H), 10.23 (s, 1H), 9.61 (d, J=7.6 Hz, 1H), 8.85 (s, 1H), 7.59-7.45 (m, 2H), 7.27-7.18 (m, 1H), 7.04 (d, J=8.8 Hz, 1H), 6.98-6.88 (m, 2H), 6.86-6.75 (m, 2H), 5.50-5.39 (m, 1H), 3.74-3.62 (m, 4H), 3.27-3.18 (m, 5H), 2.75-2.64 (m, 4H), 2.40 (s, 3H), 2.27-2.19 (m, 2H), 2.15-2.07 (m, 3H), 1.83-1.74 (m, 6H), 1.62-1.53 (m, 3H), 1.43 (s, 9H), 1.28-1.16 (m, 4H); [M+H]⁺=856.8.

### Example 29: 5-(tert-butyl)-N-((1R)-1-(4-(7-(4-(1-(4-(2,6-dioxopiperidin-3-yl)phenyl)piperidin-4-yl)piperazin-1-yl)-9H-pyrimido[4,5-b]indol-4-yl)-3-fluoro-2-methylphenyl)ethyl)-1,2,4-oxadiazole-3-carboxamide

### Step 1: 2,6-bis(benzyloxy)-3-(4-bromophenyl)pyridine

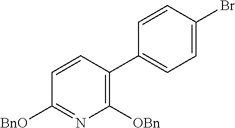 [SMILES: Brc1ccc(-c2ccc(OCc3ccccc3)nc2OCc2ccccc2)cc1]

To a solution of 2,6-bis(benzyloxy)-3-(4,4,5,5-tetramethyl-1,3,2-dioxaborolan-2-yl)pyridine (36 g, 86.3 mmol), 1-bromo-4-iodobenzene (24.3 g, 86.3 mmol) and K₂CO₃ (23.8 g, 172.6 mmol) dissolved in dioxane (500 mL) and water (100 mL) was added Pd(dppf)Cl₂ (6.32 g, 8.63 mmol). The mixture was stirred at 80° C. for 16 h and cooled down to rt. The mixture was washed with water and extracted with EtOAc. The organic layers were combined, dried over Na₂SO₄, filtered and concentrated to dryness. The residue was purified by silica gel column chromatography (PE:EtOAc=100/1) to afford the product (30 g, 78%). [M+H]⁺=446.1.

### Step 2: 8-(4-(2,6-bis(benzyloxy)pyridin-3-yl)phenyl)-1,4-dioxa-8-azaspiro[4.5]decane

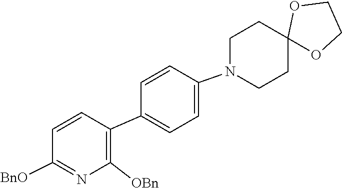 [SMILES: c1ccc(COc2ccc(-c3ccc(N4CCC5(CC4)OCCO5)cc3)c(OCc3ccccc3)n2)cc1]

To a solution of 2,6-bis(benzyloxy)-3-(4-bromophenyl)pyridine (0.89 g, 2.0 mmol), 1,4-dioxa-8-azaspiro[4.5]decane (0.29 g, 2.0 mmol), Cs₂CO₃ (1.3 g, 4.0 mmol) and Xantphos (0.23 g, 0.4 mmol) dissolved in dioxane (25 mL) was added Pd₂dba₃ (0.18 g, 0.2 mmol). The mixture was stirred at 90° C. for 16 h and cooled down to rt. The solution was washed with water and extracted with EtOAc. The organic layer was combined, dried over Na₂SO₄, filtered and concentrated to dryness. The residue was purified by silica gel column chromatography (PE:EtOAc=4/1) to afford the product (300 mg, 30%). [M+H]⁺=509.3.

### Step 3: 1-(4-(2,6-bis(benzyloxy)pyridin-3-yl)phenyl)piperidin-4-one

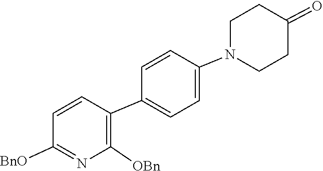 [SMILES: O=C1CCN(c2ccc(-c3ccc(OCc4ccccc4)nc3OCc3ccccc3)cc2)CC1]

To a solution of 8-(4-(2,6-bis(benzyloxy)pyridin-3-yl)phenyl)-1,4-dioxa-8-azaspiro[4.5]decane (300 mg, 0.59 mmol) in H₂O (5 mL) was added HCl/dioxane (4 N, 5 mL) dropwise. The mixture was stirred at 60° C. for 16 h. The mixture was washed by water and extracted with DCM. The organic layers were combined and dried over Na₂SO₄. The mixture was filtered and the filtrate was concentrated in vacuum to afford the product (0.2 g, 73%). [M+H]⁺=465.2.

### Step 4: 3-(4-(4-oxopiperidin-1-yl)phenyl)piperidine-2,6-dione

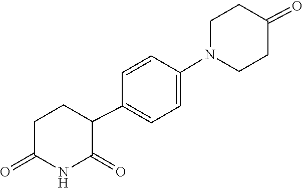 [SMILES: O=C1CCN(c2ccc(C3CCC(=O)NC3=O)cc2)CC1]

To a mixture of 1-(4-(2,6-bis(benzyloxy)pyridin-3-yl)phenyl)piperidin-4-one (0.2 g, 0.43 mmol) dissolved in THF/MeOH was added Pd/C (40 mg, 20% ow/w). The reaction was stirred at rt for 16 h under hydrogen atmosphere. The mixture was filtered by Celite pad. The filtrate was concentrated in vacuum to obtain the product (105 mg, 85%). [M+H]⁺=287.3.

### Step 5: 5-(tert-butyl)-N-((1R)-1-(4-(7-(4-(1-(4-(2,6-dioxopiperidin-3-yl)phenyl)piperidin-4-yl)piperazin-1-yl)-9H-pyrimido[4,5-b]indol-4-yl)-3-fluoro-2-methylphenyl)ethyl)-1,2, A-oxadiazole-3-carboxamide

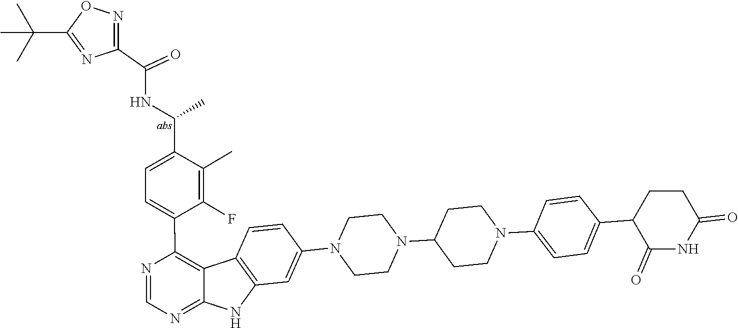 [SMILES: Cc1c([C@@H](C)NC(=O)c2noc(C(C)(C)C)n2)ccc(-c2ncnc3[nH]c4cc(N5CCN(C6CCN(c7ccc(C8CCC(=O)NC8=O)cc7)CC6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (44.3 mg, 0.540 mmol) was added. The mixture was stirred at room temperature for 5 mins. Then 3-(4-(4-oxopiperidin-1-yl)phenyl)piperidine-2,6-dione (61.7 mg, 0.216 mmol) and HOAc (0.06 mL) were added. The mixture was stirred at room temperature overnight. Then NaBH(OAc)₃ (191 mg, 0.9 mmol) was added and stirred at room temperature overnight. Then NaBH₃CN (113 mg, 1.8 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (20.16 mg, 14%). ¹H NMR (500 MHz, DMSO) δ 12.27 (d, J=70.0 Hz, 1H), 10.78 (s, 1H), 9.62 (s, 1H), 8.87 (d, J=20.0 Hz, 1H), 7.60-7.45 (m, 2H), 7.24 (d, J=30.0 Hz, 1H), 7.12-6.99 (m, 3H), 6.99-6.86 (m, 3H), 5.51-5.40 (m, 1H), 3.99 (d, J=10.0 Hz, 1H), 3.89 (d, J=10.0 Hz, 1H), 3.80-3.65 (m, 3H), 3.26-3.07 (m, 4H), 2.74-2.60 (m, 5H), 2.54 (s, 1H), 2.48-2.43 (m, 1H), 2.40 (s, 3H), 2.23-2.09 (m, 2H), 2.05-1.95 (m, 1H), 1.92-1.84 (m, 1H), 1.75 (s, 1H), 1.56 (d, J=5.0 Hz, 3H), 1.43 (s, 9H); [M+H]⁺=827.6.

### Example 30: 5-(tert-butyl)-N-((1R)-1-(4-(7-(4-((3-(4-(2,6-dioxopiperidin-3-yl)phenyl)cyclobutyl)methyl)piperazin-1-yl)-9H-pyrimido[4,5-b]indol-4-yl)-3-fluoro-2-methylphenyl)ethyl)-1,2,4-oxadiazole-3-carboxamide

### Step 1: (3-(4-bromophenyl)cyclobutyl)methanol

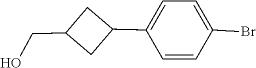 [SMILES: OCC1CC(c2ccc(Br)cc2)C1]

To a solution of 3-(4-bromophenyl)cyclobutane-1-carboxylic acid (510 mg, 2 mmol) dissolved in THF was dropwise added BH₃ (2.6 mL, 1 N in THF) at 0° C. The mixture was stirred at rt for 16 h. The reaction was quenched by 1 N HCl at 0° C. and extracted with EtOAc. The organic layers were combined and dried over Na₂SO₄. The mixture was filtered. The filtrate was concentrated to afford the product (470 mg, 98%). ¹H NMR (400 MHz, DMSO) δ 7.48 (t, J=9.2 Hz, 2H), 7.21 (dd, J=25.4, 10.6 Hz, 2H), 6.52 (s, 1H), 3.38 (d, J=16.9 Hz, 4H), 2.41-2.26 (m, 2H), 2.14 (dd, J=21.7, 10.3 Hz, 1H), 1.85-1.74 (m, 1H).

### Step 2: (3-(4-(2,6-bis(benzyloxy)pyridin-3-yl)phenyl)cyclobutyl)methanol

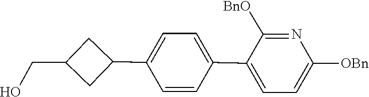 [SMILES: OCC1CC(c2ccc(-c3ccc(OCc4ccccc4)nc3OCc3ccccc3)cc2)C1]

To a mixture of (3-(4-bromophenyl)cyclobutyl)methanol (470 mg, 1.95 mmol), 2,6-bis(benzyloxy)-3-(4,4,5,5-tetramethyl-1,3,2-dioxaborolan-2-yl)pyridine (813 mg, 1.95 mmol) and Pd(PPh₃)₄ (116 mg, 0.1 mmol) dissolved in dioxane/H₂O was added Na₂CO₃ (413 mg, 3.9 mmol). The mixture was stirred at 105° C. overnight. The solvent was removed by reduced pressure. The mixture was washed by water and extracted with EtOAc. The organic layers were combined, dried over Na₂SO₄ and concentrated. The residue was purified by silica gel column chromatography (PE:EtOAc=4/1) to obtain the product (600 mg, 68%). [M+H]⁺=452.1.

### Step 3: 3-(4-(3-(hydroxymethyl)cyclobutyl)phenyl)piperidine-2,6-dione

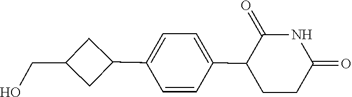 [SMILES: O=C1CCC(c2ccc(C3CC(CO)C3)cc2)C(=O)N1]

To a solution of (3-(4-(2,6-bis(benzyloxy)pyridin-3-yl)phenyl)cyclobutyl)methanol (600 mg, 1.33 mmol) dissolved in THF/MeOH was added Pd/C (60 mg, 10% ow/w). The reaction was stirred at rt for 16 h under hydrogen atmosphere. The mixture was filtered by Celite pad. The filtrate was concentrated in vacuum to afford the product (340 mg, 94%). [M+H]⁺=274.2.

### Step 4: 3-(4-(2,6-dioxopiperidin-3-yl)phenyl)cyclobutane-1-carbaldehyde

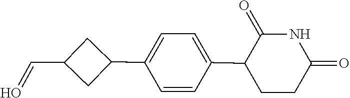 [SMILES: O=CC1CC(c2ccc(C3CCC(=O)NC3=O)cc2)C1]

To a mixture of 3-(4-(3-(hydroxymethyl)cyclobutyl)phenyl)piperidine-2,6-dione (340 mg, 1.24 mmol) dissolved in DMSO was added IBX (444 mg, 1.59 mmol). The mixture was stirred at room temperature for 4 h. Then the mixture was washed by water and extracted with DCM. The organic layers were combined and washed with brine. The mixture was dried over Na₂SO₄ and filtered. The filtrate was concentrated in vacuum. The residue was purified by silica gel column chromatography to afford the product (302 mg, 90%). [M+H]⁺=272.3.

### Step 5: 5-(tert-butyl)-N-((1R)-1-(4-(7-(4-((3-(4-(2,6-dioxopiperidin-3-yl)phenyl)cyclobutyl)methyl)piperazin-1-yl)-9H-pyrimido[4,5-b]indol-4-yl)-3-fluoro-2-methylphenyl)ethyl)-1,2,4-oxadiazole-3-carboxamide

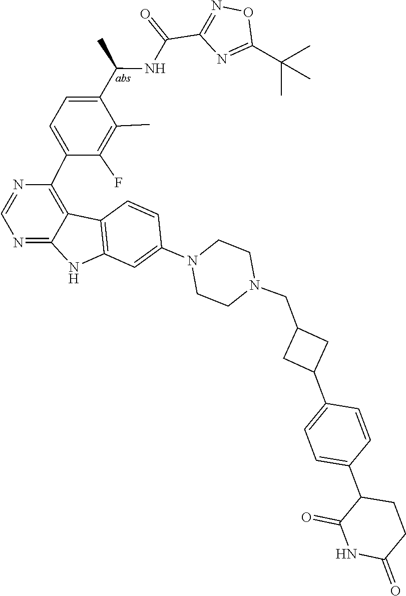 [SMILES: Cc1c([C@@H](C)NC(=O)c2noc(C(C)(C)C)n2)ccc(-c2ncnc3[nH]c4cc(N5CCN(CC6CC(c7ccc(C8CCC(=O)NC8=O)cc7)C6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (44.3 mg, 0.540 mmol) was added. The mixture was stirred at room temperature for 5 mins. Then 3-(4-(2,6-dioxopiperidin-3-yl)phenyl)cyclobutane-1-carbaldehyde (58.8 mg, 0.216 mmol) and HOAc (0.06 mL) were added. The mixture was stirred at room temperature overnight. Then NaBH(OAc)₃ (191 mg, 0.9 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-TLC (DCM:MeOH=10:1) to afford the desired product (28.04 mg, 19%). ¹H NMR (500 MHz, DMSO) δ 12.27 (d, J=70.0 Hz, 1H), 10.82 (s, 1H), 9.61 (s, 1H), 8.89 (s, 1H), 7.65-7.43 (m, 2H), 7.29-7.06 (m, 5H), 7.05-6.87 (d, J=47.3 Hz, 2H), 5.51-5.34 (m, 1H), 3.94 (s, 1H), 3.82 (d, J=5.0 Hz, 1H), 3.63-3.49 (m, 1H), 3.48-3.39 (s, 1H), 3.30 (s, 3H), 3.25-3.09 (m, 4H), 2.72-2.62 (m, 1H), 2.54 (s, 1H), 2.48-2.43 (m, 2H), 2.40 (s, 3H), 2.23-2.13 (m, 1H), 2.05-1.97 (m, 1H), 1.95-1.87 (s, 1H), 1.75 (s, 1H), 1.56 (d, J=5.0 Hz, 3H), 1.43 (s, 9H); [M+H]⁺=812.3.

### Example 31: 5-(tert-butyl)-N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-3-fluoro-2-methylphenyl)ethyl)-1,2,4-oxadiazole-3-carboxamide

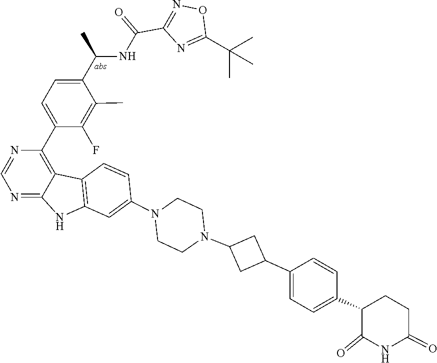 [SMILES: Cc1c([C@@H](C)NC(=O)c2noc(C(C)(C)C)n2)ccc(-c2ncnc3[nH]c4cc(N5CCN(C6CC(c7ccc([C@@H]8CCC(=O)NC8=O)cc7)C6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (44 mg, 0.540 mmol) was added. The mixture was stirred at room temperature for 5 mins. Then (S)-3-(4-(3-oxocyclobutyl)phenyl)piperidine-2,6-dione (46 mg, 0.180 mmol) and HOAc (0.06 mL) were added. The mixture was stirred at room temperature overnight. Then NaBH(OAc)₃ (190 mg, 0.9 mmol) was added and stirred at room temperature overnight. Then NaBH₃CN (226 mg, 3.6 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (55.99 mg, 39%). ¹H NMR (500 MHz, DMSO) δ 12.27 (d, J=70.0 Hz, 1H), 10.83 (s, 1H), 9.61 (d, J=5.0 Hz, 1H), 8.87 (d, J=15.0 Hz, 1H), 7.64-7.46 (m, 2H), 7.37 (s, 1H), 7.33-7.13 (m, 4H), 6.98 (d, J=45.0 Hz, 2H), 5.48-5.41 (m, 1H), 3.98 (d, J=10.0 Hz, 1H), 3.89-3.69 (m, 2H), 3.50 (s, 1H), 3.28-3.14 (m, 4H), 3.07 (s, 1H), 2.74-2.61 (m, 2H), 2.47 (s, 3H), 2.40 (s, 3H), 2.19 (d, J=10.0 Hz, 1H), 2.06-1.99 (m, 1H), 1.93-1.83 (m, 1H), 1.57 (d, J=5.0 Hz, 3H), 1.43 (s, 9H); [M+H]⁺=796.5.

### Example 32: 5-(tert-butyl)-N—((R)-1-(4-(7-(4-(3-(4-((R)-2,6-dioxopiperidin-3-yl)phenyl)cyclobutyl)piperazin-1-yl)-9H-pyrimido[4,5-b]indol-4-yl)-3-fluoro-2-methylphenyl)ethyl)-1,2,4-oxadiazole-3-carboxamide

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (44 mg, 0.540 mmol) was added. The mixture was stirred at room temperature for 5 mins. Then (R)-3-(4-(3-oxocyclobutyl)phenyl)piperidine-2,6-dione (46 mg, 0.180 mmol) and HOAc (0.06 mL) were added. The mixture was stirred at room temperature overnight. Then NaBH(OAc)₃ (190 mg, 0.9 mmol) was added and stirred at room temperature overnight. Then NaBH₃CN (226 mg, 3.6 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (50.86 mg, 35%). ¹H NMR (500 MHz, DMSO) δ 12.27 (d, J=70.0 Hz, 1H), 10.83 (s, 1H), 9.61 (d, J=10.0 Hz, 1H), 8.87 (d, J=20.0 Hz, 1H), 7.61-7.42 (m, 2H), 7.37 (s, 1H), 7.28-7.12 (s, 4H), 7.05-6.88 (m, 2H), 5.46-5.33 (m, 1H), 3.97 (s, 1H), 3.88-33.69 (m, 2H), 3.50 (s, 1H), 3.25 (s, 3H), 3.08 (s, 2H), 2.69-2.61 (m, 2H), 2.46-2.43 (m, 3H), 2.40 (s, 3H), 2.18 (d, J=5.0 Hz, 1H), 2.05-1.97 (m, 1H), 1.90-1.82 (m, 1H), 1.56 (d, J=5.0 Hz, 3H), 1.43 (s, 9H); [M+H]⁺=796.6.

### Example 33: (R)-5-(tert-butyl)-N-(1-(4-(7-(4-(3-(4-(2,4-dioxotetrahydropyrimidin-1(2H)-yl)phenyl)cyclobutyl)piperazin-1-yl)-9H-pyrimido[4,5-b]indol-4-yl)-3-fluoro-2-methylphenyl)ethyl)-1,2,4-oxadiazole-3-carboxamide

### Step 1: 1-(4-chlorobenzoyl)pyrimidine-2,4(1H,3H)-dione

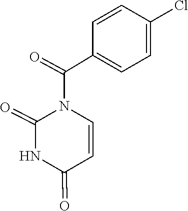 [SMILES: O=C(c1ccc(Cl)cc1)n1ccc(=O)[nH]c1=O]

To a suspension of pyrimidine-2,4(1H,3H)-dione (9 g, 0.080 mol) in DMF (60 mL), pyridine (12.9 mL) and 4-chlorobenzoyl chloride (11.1 mL, 0.081 mol) were added. The mixture was stirred at room temperature overnight. Then the mixture was filtered. The residue was washed with PE:EA=10:1, and filtered to give the product (15.16 g, 82%). [M+Na]⁺=273.0.

### Step 2: 3-((2-(trimethylsilyl)ethoxy)methyl)pyrimidine-2,4(1H,3H)-dione

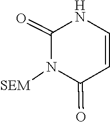 [SMILES: C[Si](C)(C)CCOCn1c(=O)cc[nH]c1=O]

To a suspension of 1-(4-chlorobenzoyl)pyrimidine-2,4(1H,3H)-dione (15 g, 0.060 mol) in DMF (60 mL), NaH (3 g, 0.075 mol) and 2-(trimethylsilyl)ethoxymethyl chloride (12 g, 0.072 mol) were added at 0° C. The mixture was stirred at room temperature overnight. The resulting solution was extracted with EtOAc (2×200.0 mL) and the organic layers were combined. The organic layer was dried over anhydrous sodium sulfate and concentrated under vacuum. The residue was dissolved in MeOH (200 mL), and K₂CO₃ (22.3 g, 0.162 mol) was added. The mixture was stirred at room temperature for 2 hours and concentrated in vacuo. Saturated aqueous NH₄Cl solution was added, and the product was extracted with EtOAc (2×200.0 mL). The combined organic fractions were dried over Na₂SO₄, filtered, and concentrated in vacuo. The crude product was purified by flash chromatography (PE:EA=100%:0%˜80%:20%) to give the product (10.9 g, 75%). [M+Na]⁺=265.0.

### Step 3: 3-(4-(4,4,5,5-tetramethyl-1,3,2-dioxaborolan-2-yl)phenyl)cyclobutan-1-one

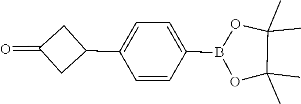 [SMILES: CC1(C)OB(c2ccc(C3CC(=O)C3)cc2)OC1(C)C]

A mixture of 3-(4-bromophenyl)cyclobutan-1-one (1 g, 0.0044 mol) and 4,4,4′,4′,5,5,5′,5′-octamethyl-2,2′-bi(1,3,2-dioxaborolane) (1.69 g, 0.0066 mol), Pd(dppf)Cl₂ (0.16 g, 0.00022 mol) and K₂CO₃ (0.86 g, 0.0088 mol) in 1,4-dioxane (20 mL) was stirred in a round bottom flask at 93° C. overnight. The mixture was evaporated in vacuum to afford the crude product, which was further purified with silica gel column chromatography (PE:EA=100:0˜75%: 25% gradient elution) to give the product (5 g, 70%). [M+H]+=273.2.

### Step 4: (4-(3-oxocyclobutyl)phenyl)boronic acid

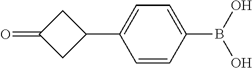 [SMILES: O=C1CC(c2ccc(B(O)O)cc2)C1]

To a solution of 3-(4-(4,4,5,5-tetramethyl-1,3,2-dioxaborolan-2-yl)phenyl)cyclobutan-1-one (1 g, 0.00368 mol) in THF (16 mL) and water (4 mL), NaIO₄ (2.3 g, 0.0110 mol) was added. The mixture was stirred at room temperature for 30 mins. Then HCl (2 M, 2 mL) was added. The mixture was stirred at room temperature overnight. The resulting solution was extracted with DCM (2×20 mL) and the organic layers were combined. The organic layer was dried over anhydrous sodium sulfate and concentrated under vacuum. The residue was purified by silica gel column chromatography (DCM:MeOH=100%: 0%˜90%: 10% gradient elution) to give the product (0.5 g, 72%). [M+H]⁺=191.0.

### Step 5: 1-(4-(3-oxocyclobutyl)phenyl)-3-((2-(trimethylsilyl)ethoxy)methyl)pyrimidine-2,4(1H,3H)-dione

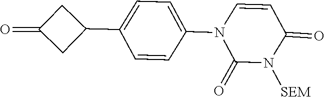 [SMILES: C[Si](C)(C)CCOCn1c(=O)ccn(-c2ccc(C3CC(=O)C3)cc2)c1=O]

A mixture of (4-(3-oxocyclobutyl)phenyl)boronic acid (1.78 g, 0.00937 mol), 3-((2-(trimethylsilyl)ethoxy)methyl)pyrimidine-2,4(1H,3H)-dione (3.4 g, 0.0140 mol), Cu(OAc)₂ (5.1 g, 0.028 mol), pyridine (3.7 g, 0.0468 mol) and 4A MS (2 g) in DMA (50 mL) was stirred in a round bottom flask at 80° C. for 3 hours. The mixture was filtered, and the filtration was extracted with EA (2×100 mL). The organic layers were combined, dried over anhydrous sodium sulfate and concentrated under vacuum. The residue was purified by silica gel column chromatography (DCM:MeOH=100%: 0%) to give the product (3.27 g, crude). [M+Na]⁺=409.2.

### Step 6: 1-(4-(3-oxocyclobutyl)phenyl)pyrimidine-2,4(1H,3H)-dione

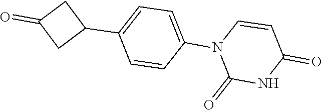 [SMILES: O=C1CC(c2ccc(-n3ccc(=O)[nH]c3=O)cc2)C1]

To a solution of 1-(4-(3-oxocyclobutyl)phenyl)-3-((2-(trimethylsilyl)ethoxy)methyl)pyrimidine-2,4(1H,3H)-dione (3.2 g, 0.0083 mol) in DCM (10 mL), TFA (10 mL) was added. The mixture was stirred at room temperature for 2 hours. Then the mixture was concentrated in vacuo and diluted with MeOH (20 mL). NH₃ (7M in MeOH, 3 mL) was added. The mixture was stirred at room temperature for 1 hour and concentrated in vacuo. The residue was purified by silica gel column chromatography (DCM:MeOH=100%: 0%˜90%:10% gradient elution) to give the product (1.1 g, 50%). [M+H]⁺=257.0.

### Step 7: 1-(4-(3-oxocyclobutyl)phenyl)dihydropyrimidine-2,4(1H,3H)-dione

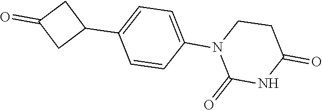 [SMILES: O=C1CC(c2ccc(N3CCC(=O)NC3=O)cc2)C1]

To a solution of 1-(4-(3-oxocyclobutyl)phenyl)pyrimidine-2,4(1H,3H)-dione (300 mg, 1.167 mmol) in THF (10 mL), Pd/C (50 mg, 10%) was added. The mixture was stirred at room temperature overnight under H₂. Then the mixture was filtered, and the filtration was concentrated in vacuo. The residue was purified by silica gel column chromatography (DCM:MeOH=100%: 0%˜90%:10% gradient elution) to give the product (40 mg, 13%). [M+H]⁺=259.1.

### Step 13: (R)-5-(tert-butyl)-N-(1-(4-(7-(4-(3-(4-(2,4-dioxotetrahydropyrimidin-1(2H)-yl)phenyl)cyclobutyl)piperazin-1-yl)-9H-pyrimido[4,5-b]indol-4-yl)-3-fluoro-2-methylphenyl)ethyl)-1,2,4-oxadiazole-3-carboxamide

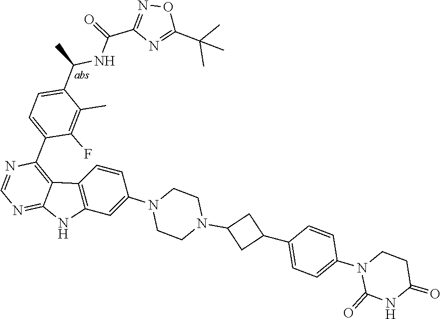 [SMILES: Cc1c([C@@H](C)NC(=O)c2noc(C(C)(C)C)n2)ccc(-c2ncnc3[nH]c4cc(N5CCN(C6CC(c7ccc(N8CCC(=O)NC8=O)cc7)C6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (70 mg, crude) in dichloromethane (5 mL) and MeOH (5 mL), NaOAc (31 mg, 0.375 mmol) was added. The mixture was stirred at room temperature for 5 mins. Then 1-(4-(3-oxocyclobutyl)phenyl)dihydropyrimidine-2,4(1H,3H)-dione (40 mg, 0.125 mmol) and HOAc (0.06 mL) were added. The mixture was stirred at room temperature overnight. Then NaBH(OAc)₃ (133 mg, 0.630 mmol) was added and stirred at room temperature overnight. Then NaBH₃CN (80 mg, 1.25 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-TLC (DCM:MeOH=10:1) to afford the desired product (7.3 mg, 5%). ¹H NMR (500 MHz, DMSO) δ 12.19 (s, 1H), 10.33 (s, 1H), 9.59 (d, J=5.0 Hz, 1H), 8.85 (s, 1H), 7.58-7.48 (m, 2H), 7.38-7.16 (m, 5H), 6.94 (s, 2H), 5.48-5.39 (m, 1H), 3.76 (t, J=5.0 Hz, 2H), 3.3-3.12 (m, 6H), 2.75 (s, 1H), 2.69 (d, J=5.0 Hz, 3H), 2.62-2.52 (m, 4H), 2.48-2.44 (m, 1H), 2.40 (s, 3H), 1.89 (s, 1H), 1.56 (d, J=6.8 Hz, 3H), 1.43 (s, 9H); [M+H]⁺=799.6.

### Example 34: 5-(tert-butyl)-N-((1R)-1-(4-(7-(4-(1-(3-(2,6-dioxopiperidin-3-yl)phenyl)piperidin-4-yl)piperazin-1-yl)-9H-pyrimido[4,5-b]indol-4-yl)-3-fluoro-2-methylphenyl)ethyl)-1,2,4-oxadiazole-3-carboxamide

### Step 1: 8-(3-bromophenyl)-1,4-dioxa-8-azaspiro[4.5]decane

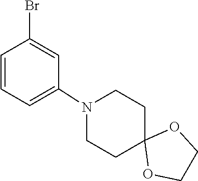 [SMILES: Brc1cccc(N2CCC3(CC2)OCCO3)c1]

To a solution of 1-bromo-3-iodobenzene (2.83 g, 10 mmol), 1,4-dioxa-8-azaspiro[4.5]decane (1.43 g, 10 mmol), CuI (0.389 g, 2 mmol) and L-proline (0.23 g, 2 mmol) dissolved in NMP was added K₂CO₃ (2.76 g, 20 mmol). The mixture was stirred at 110° C. for 16 h. The mixture was cooled down to rt. The mixture was washed with water and extracted with EtOAc. The organic layers were combined and washed with brine. The mixture was dried over Na₂SO₄, filtered and concentrated to dryness. The residue was purified by silica gel column chromatography (PE:EtOAc=4/1) to afford the product (1.01 g, 33%). [M+H]⁺=298.1.

### Step 2: 1-(3-bromophenyl)piperidin-4-one

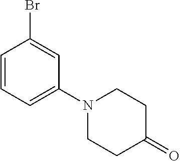 [SMILES: O=C1CCN(c2cccc(Br)c2)CC1]

To a solution of 8-(3-bromophenyl)-1,4-dioxa-8-azaspiro[4.5]decane (1.01 g, 3.4 mmol) and 1,4-dioxa-8-azaspiro[4.5]decane (0.29 g, 2.0 mmol) in H₂O was dropwise added HCl (4 M, 6.8 mL, 27.2 mmol). The mixture was stirred at 50° C. for 16 h. The mixture was cooled down to rt. The solution was washed with water and extracted with EtOAc. The organic layer was combined, dried over Na₂SO₄, filtered and concentrated to afford the product (822 mg, 96%). [M+H]⁺=254.1.

### Step 3: 1-(3-(2,6-bis(benzyloxy)pyridin-3-yl)phenyl)piperidin-4-one

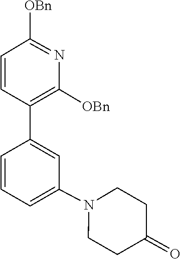 [SMILES: O=C1CCN(c2cccc(-c3ccc(OCc4ccccc4)nc3OCc3ccccc3)c2)CC1]

To a solution of 1-(3-bromophenyl)piperidin-4-one (254 mg, 1 mmol), 2,6-bis(benzyloxy)-3-(4,4,5,5-tetramethyl-1,3,2-dioxaborolan-2-yl)pyridine (419 mg, 1 mmol) and Pd(PPh₃)₄ (116 mg, 0.1 mmol) dissolved in dioxane/H₂O was added K₂CO₃ (276 mg, 2 mmol). The mixture was stirred at 105° C. overnight. The mixture was washed with water and extracted with EtOAc. The organic layers were combined, dried over Na₂SO₄, filtered and concentrated to dryness. The residue was purified by silica gel column chromatography (PE:EtOAc=4/1) to afford the product (0.28 g, 61%). [M+H]⁺=465.2.

### Step 4: 3-(3-(4-oxopiperidin-1-yl)phenyl)piperidine-2,6-dione

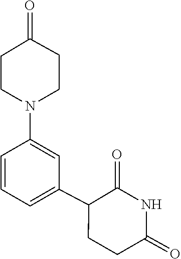 [SMILES: O=C1CCN(c2cccc(C3CCC(=O)NC3=O)c2)CC1]

To a mixture of 1-(3-(2,6-bis(benzyloxy)pyridin-3-yl)phenyl)piperidin-4-one (0.28 g, 0.61 mmol) dissolved in THF/MeOH was added Pd/C (30 mg, 10% w/w). The reaction was stirred at rt for 16 h under hydrogen atmosphere. The mixture was filtered by Celite pad. The filtrate was concentrated in vacuum to obtain the product (168 mg, 97%). [M+H]⁺=287.3.

### Step 5: 5-(tert-butyl)-N-((1R)-1-(4-(7-(4-(1-(3-(2,6-dioxopiperidin-3-yl)phenyl)piperidin-4-yl)piperazin-1-yl)-9H-pyrimido[4,5-b]indol-4-yl)-3-fluoro-2-methylphenyl)ethyl)-1,2,4-oxadiazole-3-carboxamide

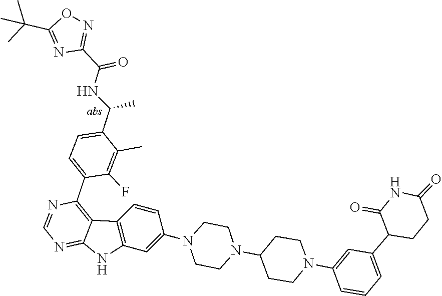 [SMILES: Cc1c([C@@H](C)NC(=O)c2noc(C(C)(C)C)n2)ccc(-c2ncnc3[nH]c4cc(N5CCN(C6CCN(c7cccc(C8CCC(=O)NC8=O)c7)CC6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (67.8 mg, crude) and 3-(3-(4-oxopiperidin-1-yl)phenyl)piperidine-2,6-dione (35 mg, 0.122 mmol) in dichloromethane (5 mL) and MeOH (5 mL), NaOAc (30 mg, 0.366 mmol) was added. The mixture was stirred at room temperature for 5 mins. Then HOAc (0.06 mL) was added. The mixture was stirred at 30° C. overnight. Then NaBH(OAc)₃ (129 mg, 0.610 mmol) was added and stirred at room temperature overnight. Then NaBH₃CN (78 mg, 1.22 mmol) was added and stirred at 30° C. overnight. The reaction mixture was concentrated in vacuum, and the residue was purified by silica gel column chromatography (DCM:MeOH=100%: 0% 90%: 10% gradient elution) to give the crude product, which was further purified by prep-TLC (DCM:MeOH=10:1) to afford the desired product (16.31 mg, 16%). ¹H NMR (500 MHz, DMSO) δ 12.34 (s, 1H), 10.81 (s, 1H), 9.62 (s, 1H), 9.52 (s, 1H), 8.90 (s, 1H), 7.60-7.45 (m, 2H), 7.26 (d, J=5.0 Hz, 1H), 7.21-7.13 (m, 2H), 7.11-7.05 (m, 1H), 7.03 (s, 1H), 7.00-6.95 (m, 1H), 6.91-6.79 (m, 2H), 6.64 (d, J=10.0 Hz, 1H), 5.49-5.38 (m, 1H), 4.00 (d, J=10.0 Hz, 2H), 3.90 (d, J=10.0 Hz, 2H), 3.80-3.74 (m, 1H), 3.72-3.64 (d, J=10.0 Hz, 2H), 3.27-3.21 (m, 2H), 3.13-3.02 (m, 2H), 2.77-2.69 (m, 2H), 2.67-2.59 (m, 1H), 2.41 (s, 3H), 2.26-2.12 (m, 3H), 2.06-1.96 (m, 1H), 1.78-1.68 (m, 2H), 1.57 (d, J=5.0 Hz, 3H), 1.44 (s, 9H); [M+H]⁺=827.5.

### Example 35: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: benzyl(R)-4-(4-(3-methyl-4-(1-(3-(1-methylcyclopropyl)-1,2,4-oxadiazole-5-carboxamido)ethyl)phenyl)-9H-pyrimido[4,5-b]indol-7-yl)piperazine-1l-carboxylate

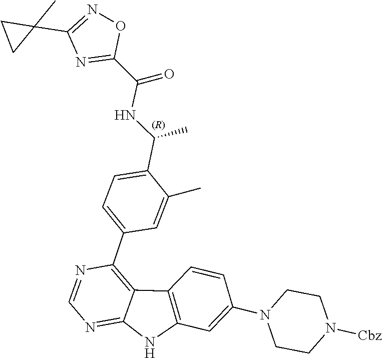

A mixture of benzyl 4-(4-chloro-9H-pyrimido[4,5-b]indol-7-yl)piperazine-1-carboxylate (0.2 g, 0.47 mmol), (R)—N-(1-(2-methyl-4-(4,4,5,5-tetramethyl-1,3,2-dioxaborolan-2-yl)phenyl)ethyl)-3-(1-methylcyclopropyl)-1,2,4-oxadiazole-5-carboxamide (the compound was obtained through the same way in WO2021219070A) (191 mg, 0.47 mmol), Pd(dppf)Cl₂ (38 mg, 0.047 mmol) and Cs₂CO₃ (0.305 g, 0.94 mmol) in 1,4-dioxane (20 mL) and H₂O (40 mL) was stirred in a round bottom flask at 90° C. overnight. The mixture was evaporated in vacuum to afford the crude product, which was further purified with silica gel column chromatography (PE:EA=100:0˜3:1 gradient elution) to give the product (0.2 g, 63.5%). [M+H]⁺=671.8.

### Step 2: (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide trifluoroacetate

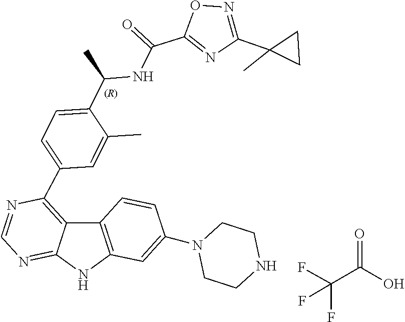

A mixture of benzyl (R)-4-(4-(3-methyl-4-(1-(3-(1-methylcyclopropyl)-1,2,4-oxadiazole-5-carboxamido)ethyl)phenyl)-9H-pyrimido[4,5-b]indol-7-yl)piperazine-1-carboxylate (0.2 g, 0.3 mmol) and trifluoroacetic acid (20 mL) in dichloromethane (20 mL) was stirred in a round bottom flask at room temperature overnight. The mixture was evaporated in vacuum to afford the crude product (0.13 g, 67%), which was used for next step without further purification. [M+H]⁺=537.6.

### Step 3: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

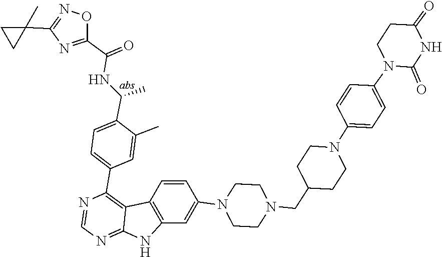 [SMILES: Cc1cc(-c2ncnc3[nH]c4cc(N5CCN(CC6CCN(c7ccc(N8CCC(=O)NC8=O)cc7)CC6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide trifluoroacetate (130 mg, 0.243 mmol) and 1-(4-(2,4-dioxotetrahydropyrimidin-1(2H)-yl)phenyl)piperidine-4-carbaldehyde (467.52 mg, 0.291 mmol) in MeOH (10 mL) was stirred in a round bottom flask for 30 min at room temperature. Then NaBH(OAc)₃ (103 mg, 0.485 mmol) was added and stirred for 2 h at room temperature. The reaction was quenched with water and the mixture was washed once with saturated aqueous NaHCO₃, then extracted with DCM. The organic layer was dried over anhydrous Na₂SO₄, and evaporated in vacuum to afford the crude product, which was further purified with pre-HPLC to give the product (100 mg, 65%). ¹H NMR (400 MHz, DMSO) δ 12.19 (s, 1H), 10.26 (s, 1H), 9.88 (d, J=7.2 Hz, 1H), 8.90-8.80 (m, 1H), 7.90-7.60 (m, 4H), 7.20-7.10 (m, 1H), 7.0-6.88 (m, 4H), 5.45-5.35 (m, 1H), 3.97-3.85 (m, 1H), 3.80-3.60 (m, 5H), 3.45-3.35 (m, 1H), 3.30-3.05 (m, 4H), 2.80-2.60 (m, 4H), 2.30-2.20 (m, 1H), 2.14-1.68 (m, 3H), 1.56 (d, J=6.4 Hz, 3H), 1.49 (s, 3H), 1.40-1.15 (m, 4H), 1.05-0.95 (m, 2H); [M+H]⁺=822.8.

### Example 36: 5-(tert-butyl)-N-((1R)-1-(4-(7-(4-((4-(4-(2,6-dioxopiperidin-3-yl)phenyl)piperidin-1-yl)methyl)piperidin-1-yl)-9H-pyrimido[4,5-b]indol-4-yl)-3-fluoro-2-methylphenyl)ethyl)-1,2,4-oxadiazole-3-carboxamide

### Step 1: 7-bromo-4-((2-(trimethylsilyl)ethoxy)methoxy)-9-((2-(trimethylsilyl)ethoxy)methyl)-9H-pyrimido[4,5-b]indole

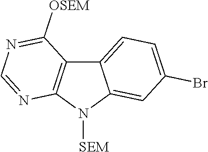

To a stirred mixture of 7-bromo-3,9-dihydro-4H-pyrimido[45-b]indol-4-one (20.0 g, 75.734 mmol) and t-BuONa (18.2 g, 189.335 mmol) in DMF (400 mL) was added SEM-Cl (40 mL, 227.218 mmol) dropwise at 0° C. under nitrogen atmosphere. The resulting mixture was stirred for 3 h at room temperature. The reaction was quenched with water at room temperature. The resulting mixture was extracted with EtOAc (2×500 mL). The combined organic layers were washed with brine (3×500 mL) and dried over anhydrous Na₂SO₄. After filtration, the filtrate was concentrated under reduced pressure. The residue was purified by silica gel column chromatography, eluted with PE/EtOAc (10:1 to 5:1) to afford the product (18.1 g, 45%). [M+H]⁺=524.1.

### Step 2: tert-butyl 4-vinylpiperidine-1-carboxylate

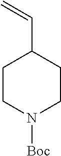

To a stirred mixture of methyltriphenylphosphanium bromide (26.8 g, 75.020 mmol) in THF (80 mL) was added t-BuOK (9.2 g, 82.522 mmol) in portions at 0° C. under nitrogen atmosphere. The resulting mixture was stirred for 1 h at room temperature under nitrogen atmosphere. To the above mixture was added tert-butyl 4-formylpiperidine-1-carboxylate (8.0 g, 37.510 mmol) in THF (80 mL) dropwise at 0° C. The resulting mixture was stirred for additional 3 h at room temperature. The reaction was quenched with water at 0° C. and concentrated under reduced pressure. The resulting mixture was extracted with EtOAc (2×500 mL). The combined organic layers were washed with brine (500 mL) and dried over anhydrous Na₂SO₄. After filtration, the filtrate was concentrated under reduced pressure. The residue was purified by silica gel column chromatography, eluted with PE/EtOAc (10:1 to 4:1) to afford the product (7 g, 88%).

### Step 3: 4-vinylpiperidine hydrochloride

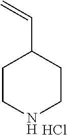

To a stirred solution of tert-butyl 4-vinylpiperidine-1-carboxylate (9.5 g, 44.959 mmol) in DCM (90 mL) was added HCl in 1,4-dioxane (4 M) (90 mL) dropwise at room temperature. The resulting mixture was stirred for 1 h at room temperature and concentrated under reduced pressure. To the resulting mixture was added MTBE (100 mL) and filtered. The filter cake was washed with MTBE (3×50 mL). This resulted in the product (6 g, 90%).

### Step 4: 4-((2-(trimethylsilyl)ethoxy)methoxy)-9-((2-(trimethylsilyl)ethoxy)methyl)-7-(4-vinylpiperidin-1-yl)-9H-pyrimido[4,5-b]indole

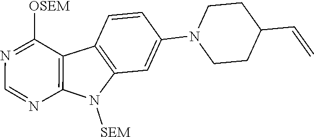

To a stirred mixture of 7-bromo-4-((2-(trimethylsilyl)ethoxy)methoxy)-9-((2-(trimethylsilyl)ethoxy)methyl)-9H-pyrimido[4,5-b]indole (9.2 g, 17.537 mmol) and 4-vinylpiperidine hydrochloride (3.8 g, 26.278 mmol) in dioxane (180 mL) were added Cs₂CO₃ (17.1 g, 52.606 mmol), XPhos (1.2 g, 2.622 mmol) and XPhos Pd G₃ (3.3 g, 3.946 mmol) at room temperature under nitrogen atmosphere. The resulting mixture was stirred overnight at 100° C. under nitrogen atmosphere and allowed to cool down to room temperature. The resulting mixture was filtered, and the filter cake was washed with EtOAc (3×100 mL). The filtrate was concentrated under reduced pressure. The residue was purified by silica gel column chromatography, eluted with PE/EtOAc (10:1 to 4:1) to afford the product (4.5 g, 46%). [M+H]⁺=555.5.

### Step 5: 7-(4-vinylpiperidin-1-yl)-3,9-dihydro-4H-pyrimido[4,5-b]indol-4-one

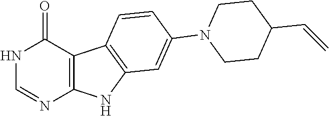 [SMILES: C=CC1CCN(c2ccc3c(c2)[nH]c2nc[nH]c(=O)c23)CC1]

A solution of 4-((2-(trimethylsilyl)ethoxy)methoxy)-9-((2-(trimethylsilyl)ethoxy)methyl)-7-(4-vinylpiperidin-1-yl)-9H-pyrimido[4,5-b]indole (2.5 g, 4.505 mmol) in TFA (30 mL) was stirred for 2 h at room temperature. The resulting mixture was concentrated under reduced pressure. To the residue was added DCM (50 mL), MeOH (10 mL) and basified to pH 8 with NH₃·H₂O. The resulting mixture was stirred for 24 h at room temperature and concentrated under reduced pressure. The resulting mixture was added water (50 mL) and filtered. The filter cake was washed with water (3×20 mL). The solid was dried under infrared light to give the product (1.1 g, 82%). [M+H]⁺=295.1.

### Step 6: 4-chloro-7-(4-vinylpiperidin-1-yl)-9H-pyrimido[4,5-b]indole

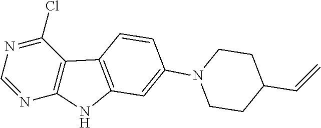 [SMILES: C=CC1CCN(c2ccc3c(c2)[nH]c2ncnc(Cl)c23)CC1]

A solution of 7-(4-vinylpiperidin-1-yl)-3,9-dihydro-4H-pyrimido[4,5-b]indol-4-one (1.1 g, 3.737 mmol) in POCl₃ (20 mL) was stirred for 2 h at 110° C. The mixture was allowed to cool down to room temperature and concentrated under reduced pressure. To the residue was added EtOAc (50 mL) and basified to pH 8 with saturated NaHCO₃ (aq.). The resulting mixture was extracted with EtOAc (3×100 mL). The combined organic layers were washed with brine (100 mL) and dried over anhydrous Na₂SO₄. After filtration, the filtrate was concentrated under reduced pressure to afford the product (1.1 g, 94%). [M+H]⁺=313.1.

### Step 7: tert-butyl 4-chloro-7-(4-vinylpiperidin-1-yl)-9H-pyrimido[4,5-b]indole-9-carboxylate

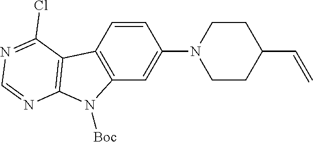 [SMILES: C=CC1CCN(c2ccc3c4c(Cl)ncnc4n(C(=O)OC(C)(C)C)c3c2)CC1]

To a stirred solution of 4-chloro-7-(4-vinylpiperidin-1-yl)-9H-pyrimido[4,5-b]indole (1.1 g, 3.517 mmol), DIEA (1.2 mL, 7.062 mmol) and DMAP (42.96 mg, 0.352 mmol) in DMF (20 mL) was added Boc₂O (1.15 g, 5.28 mmol) at 0° C. The resulting mixture was stirred for 16 h at room temperature and diluted with water (30 mL). The resulting mixture was extracted with EtOAc (3×50 mL). The combined organic layers were washed with brine (50 mL) and dried over anhydrous Na₂SO₄. After filtration, the filtrate was concentrated under reduced pressure. The residue was purified by silica gel column chromatography, eluted with PE/EtOAc (10:1 to 5:1) to afford the product (1.1 g, 82%). [M+H]⁺=413.1.

### Step 8: tert-butyl 4-chloro-7-(4-formylpiperidin-1-yl)-9H-pyrimido[4,5-b]indole-9-carboxylate

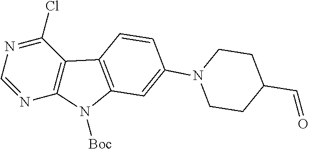 [SMILES: CC(C)(C)OC(=O)n1c2cc(N3CCC(C=O)CC3)ccc2c2c(Cl)ncnc21]

To a stirred mixture of tert-butyl 4-chloro-7-(4-vinylpiperidin-1-yl)-9H-pyrimido[4,5-b]indole-9-carboxylate (1.1 g, 2.616 mmol) and NaIO₄ (2.2 g, 10.473 mmol) in THF (20 mL) and H₂O (5 mL) was added K₂OsO₄·2H₂O (19 mg, 0.052 mmol) at room temperature. The resulting mixture was stirred for 1 h at room temperature. The reaction was quenched with saturated Na₂S₂O₃ (aq.) at room temperature. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by silica gel column chromatography, eluted with PE/EtOAc (10:1 to 2:1) to afford the product (215 mg, 20%). ¹H NMR (400 MHz, CDCl₃) δ 9.77 (s, 1H), 8.87 (s, 1H), 8.25 (d, J=8.7 Hz, 1H), 7.96 (d, J=24.0 Hz, 1H), 7.20 (s, 1H), 3.91-3.74 (m, 2H), 3.15 (t, J=11.4 Hz, 2H), 2.56 (s, 1H), 2.17 (s, 2H), 1.97-1.91 (m, 2H), 1.79 (s, 9H); [M+H]⁺=415.1.

### Step 9: (R)-5-(tert-butyl)-N-(1-(3-fluoro-4-(7-(4-formylpiperidin-1-yl)-9H-pyrimido[4,5-b]indol-4-yl)-2-methylphenyl)ethyl)-1,2,4-oxadiazole-3-carboxamide

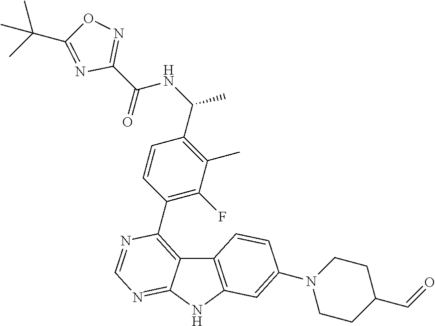 [SMILES: Cc1c([C@@H](C)NC(=O)c2noc(C(C)(C)C)n2)ccc(-c2ncnc3[nH]c4cc(N5CCC(C=O)CC5)ccc4c23)c1F]

A mixture of tert-butyl 4-chloro-7-(4-formylpiperidin-1-yl)-9H-pyrimido[4,5-b]indole-9-carboxylate (100 mg, 0.241 mmol), (R)-5-(tert-butyl)-N-(1-(3-fluoro-2-methyl-4-(4,4,5,5-tetramethyl-1,3,2-dioxaborolan-2-yl)phenyl)ethyl)-1,2,4-oxadiazole-3-carboxamide (103.5 mg, 0.241 mmol), Pd(dppf)Cl₂ (8.8 mg, 0.012 mmol) and K₂CO₃ (53 mg, 0.386 mmol) in 1,4-dioxane (10 mL) and water (2 mL) was stirred at 93° C. overnight. The mixture was concentrated in vacuum, and the residue was purified with silica gel column chromatography (PE:EA=100%: 0%˜0%: 100% gradient elution) to give the titled product (20 mg, 14%). [M+H]⁺=584.0.

### Step 10: tert-butyl 4-(4-(4,4,5,5-tetramethyl-1,3,2-dioxaborolan-2-yl)phenyl)piperidine-1-carboxylate

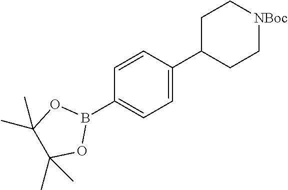 [SMILES: CC(C)(C)OC(=O)N1CCC(c2ccc(B3OC(C)(C)C(C)(C)O3)cc2)CC1]

tert-Butyl 4-(4-bromophenyl)piperidine-1-carboxylate (10 g, 29.5 mmol), (Bpin)₂ (11.3 g, 44.3 mmol), Pd(dppf)Cl₂ (3.2 g, 4.4 mmol), KOAc (5.8 g, 59 mmol) were dissolved in dioxane (400 mL). The resulting mixture was then heated under reflux for 2 h until LC-MS indicated all the starting material was consumed. The mixture was cooled to room temperature, filtered off the solid and concentrated to afford the product (12 g, crude), which was used directly without further purification. [M+H]⁺=388.3.

### Step 11: tert-butyl 4-(4-(2,6-bis(benzyloxy)pyridin-3-yl)phenyl)piperidine-1-carboxylate

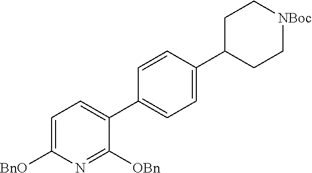 [SMILES: CC(C)(C)OC(=O)N1CCC(c2ccc(-c3ccc(OCc4ccccc4)nc3OCc3ccccc3)cc2)CC1]

Tert-butyl 4-(4-(4,4,5,5-tetramethyl-1,3,2-dioxaborolan-2-yl)phenyl)piperidine-1-carboxylate (12 g, crude), Pd(dppf)Cl₂ (3.2 g, 4.4 mmol), 2,6-bis(benzyloxy)-3-bromopyridine (16.2 g, 44.0 mmol), Cs₂CO₃ (28.7 g, 88 mmol) were placed in dioxane/water (300 mL, 10:1). The mixture was stirred at 100° C. overnight until LC-MS indicated all the starting material was consumed. The reaction was cooled to room temperature and filtered off the solid. The filtrate was concentrated and purified with SiO₂-gel column (eluted with EtOAc/hexane=1:1) to give the desired product (5 g, 30.8% over 2 steps). [M+H]⁺=551.3.

### Step 12: tert-butyl 4-(4-(2,6-dioxopiperidin-3-yl)phenyl)piperidine-1-carboxylate

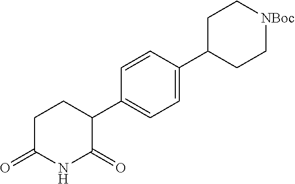 [SMILES: CC(C)(C)OC(=O)N1CCC(c2ccc(C3CCC(=O)NC3=O)cc2)CC1]

Tert-butyl 4-(4-(2,6-bis(benzyloxy)pyridin-3-yl)phenyl)piperidine-1-carboxylate (5 g, 9.1 mmol) was dissolved in MeOH (50 mL), and Pd/C (10%, w/w, 0.5 g) was added to the solution in one portion. The resulting mixture was stirred under H₂ atmosphere (1 atm) overnight until LC-MS indicated all the starting material was consumed. The mixture was filtered and concentrated to give the desired product (1.9 g, 56.1%). [M+H]⁺=373.1.

### Step 13: 3-(4-(piperidin-4-yl)phenyl)piperidine-2,6-dione hydrochloride

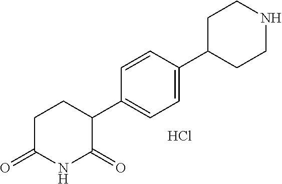 [SMILES: O=C1CCC(c2ccc(C3CCNCC3)cc2)C(=O)N1 ; Cl]

tert-Butyl 4-(4-(2,6-dioxopiperidin-3-yl)phenyl)piperidine-1-carboxylate (1.9 g, 5.1 mmol) was dissolved in HCl-dioxane (4 M, 20 mL) and the mixture was stirred at room temperature for 2 h until LC-MS indicated all the starting material was consumed. The mixture was concentrated and slurred with MTBE to afford the desired product. (1.6 g, 100%). [M+H]⁺=273.2.

### Step 14: 5-(tert-butyl)-N-((1R)-1-(4-(7-(4-((4-(4-(2,6-dioxopiperidin-3-yl)phenyl)piperidin-1-yl)methyl)piperidin-1-yl)-9H-pyrimido[4,5-b]indol-4-yl)-3-fluoro-2-methylphenyl)ethyl)-1,2,4-oxadiazole-3-carboxamide

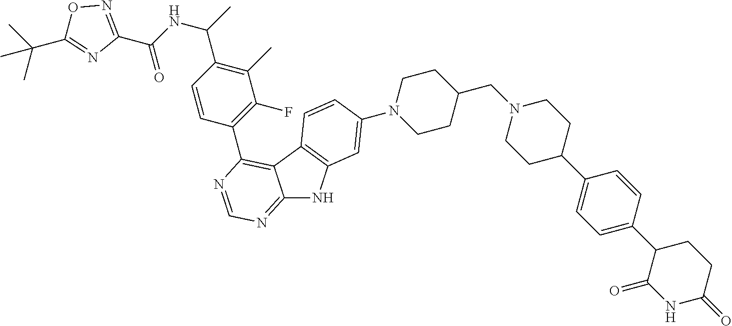 [SMILES: Cc1c(C(C)NC(=O)c2noc(C(C)(C)C)n2)ccc(-c2ncnc3[nH]c4cc(N5CCC(CN6CCC(c7ccc(C8CCC(=O)NC8=O)cc7)CC6)CC5)ccc4c23)c1F]

A mixture of (R)-5-(tert-butyl)-N-(1-(3-fluoro-4-(7-(4-formylpiperidin-1-yl)-9H-pyrimido[4,5-b]indol-4-yl)-2-methylphenyl)ethyl)-1,2,4-oxadiazole-3-carboxamide (20 mg, 0.0343 mmol), 3-(4-(piperidin-4-yl)phenyl)piperidine-2,6-dione hydrochloride (18.7 mg, 0.0686 mmol) and NaOAc (8.4 mg, 0.103 mmol) in DCM/MeOH (3 mL/3 mL) was stirred at room temperature for 30 mins. Then HOAc (0.04 mL) was added. The mixture was stirred at room temperature overnight. Then NaBH(OAc)₃ (36 mg, 0.172 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give crude product, which was further purified by prep-TLC (DCM:MeOH=10:1) to afford the desired product (8.09 mg, 28%). ¹H NMR (500 MHz, DMSO) δ 12.19 (s, 1H), 10.84 (s, 1H), 9.62 (d, J=10.0 Hz, 1H), 8.85 (s, 1H), 7.61-7.45 (m, 2H), 7.30-7.05 (m, 6H), 6.93 (s, 2H), 5.53-5.37 (m, 1H), 3.83 (d, J=10.0 Hz, 4H), 3.62 (s, 1H), 3.31 (s, 2H), 3.05 (s, 3H), 2.81 (s, 3H), 2.71-2.63 (m, 2H), 2.47-2.45 (m, 2H), 2.40 (s, 3H), 2.22-2.14 (m, 2H), 2.07-1.98 (m, 3H), 1.87 (s, 2H), 1.56 (d, J=5.0 Hz, 3H), 1.43 (s, 9H); [M+H]⁺=840.6.

### Example 37: (R)-3-(tert-butyl)-N-(5-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2,3-dihydro-1H-inden-1-yl)-1,2,4-oxadiazole-5-carboxamide

### Step 1: (R)—N-(5-bromo-2,3-dihydro-1H-inden-1-yl)-3-(tert-butyl)-1,2,4-oxadiazole-5-carboxamide

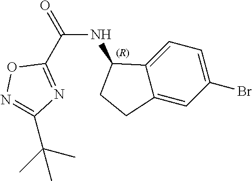

To a suspension of sodium 3-(tert-butyl)-1,2,4-oxadiazole-5-carboxylate (500 mg, 2.01 mmol) in DCM (40 mL) was added DMF (0.06 mL). Then (COCl)₂ (5.6 mL, 1 M in DCM, 5.6 mmol) was added dropwise. The mixture was stirred at rt for 1.5 hours. Then the mixture was filtered, and the filtration was concentrated in vacuo. The residue was diluted with DCM (10 mL). To a solution of (R)-5-bromo-2,3-dihydro-1H-inden-1-amine (540 mg, 2.8 mmol) in DCM (20 mL), triethylamine (1.01 g, 10.05 mmol) was added. Then the mixture above was added dropwise and stirred at room temperature overnight. The mixture was evaporated in vacuum to afford the crude product, which was further purified with silica gel column chromatography (PE:EA=100%: 0%˜50%: 50% gradient elution) to give the product (500 mg, 54%). [M+Na]⁺=386.1.

### Step 2: (R)-3-(tert-butyl)-N-(5-(4,4,5,5-tetramethyl-1,3,2-dioxaborolan-2-yl)-2,3-dihydro-1H-inden-1-yl)-1,2,4-oxadiazole-5-carboxamide

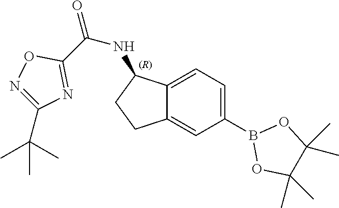

A mixture of (R)—N-(5-bromo-2,3-dihydro-1H-inden-1-yl)-3-(tert-butyl)-1,2,4-oxadiazole-5-carboxamide (180 mg, 0.467 mmol) and 4,4,4′,4′,5,5,5′,5′-octamethyl-2,2′-bi(1,3,2-dioxaborolane) (178 mg, 0.701 mmol), Pd(dppf)Cl₂ (34 mg, 0.0467 mmol) and KOAc (91.5 mg, 0.934 mmol) in 1,4-dioxane (10 mL) was stirred at 90° C. overnight under nitrogen. The mixture was evaporated in vacuum to afford the crude product, which was further purified with silica gel column chromatography (PE:EA=100%: 0%˜0%: 100% gradient elution) to give the product (280 mg, crude). [M+H]⁺=412.0.

### Step 3: benzyl (R)-4-(4-(1-(3-(tert-butyl)-1,2,4-oxadiazole-5-carboxamido)-2,3-dihydro-1H-inden-5-yl)-9H-pyrimido[4,5-b]indol-7-yl)piperazine-1-carboxylate

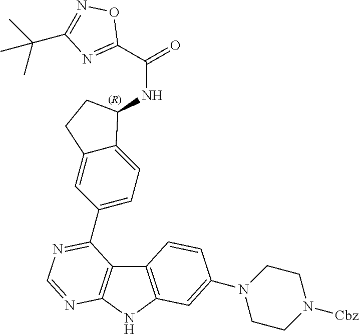

A mixture of benzyl 4-(4-chloro-9H-pyrimido[4,5-b]indol-7-yl)piperazine-1-carboxylate (286 mg, 0.679 mmol), (R)-3-(tert-butyl)-N-(5-(4,4,5,5-tetramethyl-1,3,2-dioxaborolan-2-yl)-2,3-dihydro-1H-inden-1-yl)-1,2,4-oxadiazole-5-carboxamide (280 mg, crude), Pd(dppf)Cl₂ (50 mg, 0.068 mmol) and KOAc (150 mg, 1.08 mmol) in 1,4-dioxane (10 mL) and water (2 mL) was stirred at 90° C. overnight under nitrogen. The mixture was evaporated in vacuum to afford the crude product, which was further purified with silica gel column chromatography (PE:EA=100%: 0%˜0%: 100% gradient elution) to give the titled product (120 mg, 26%). [M+H]⁺=671.0.

### Step 4: (R)-3-(tert-butyl)-N-(5-(7-(piperazin-1-yl)-9H-pyrimido[4,5-b]indol-4-yl)-2,3-dihydro-1H-inden-1-yl)-1,2,4-oxadiazole-5-carboxamide

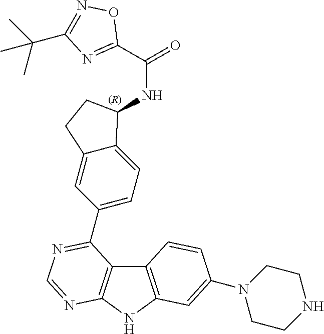

A solution of benzyl (R)-4-(4-(1-(3-(tert-butyl)-1,2,4-oxadiazole-5-carboxamido)-2,3-dihydro-1H-inden-5-yl)-9H-pyrimido[4,5-b]indol-7-yl)piperazine-1-carboxylate (120 mg, 0.223 mmol) in trifluoroacetic acid (10 mL) was stirred at 85° C. for 2 hours. The mixture was evaporated in vacuum to afford the crude product (100 mg, crude), which was used in next step without further purification. [M+H]⁺=537.0.

### Step 5: (R)-3-(tert-butyl)-N-(5-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-2,3-dihydro-1H-inden-1-vi)-1,2,4-oxadiazole-5-carboxamide

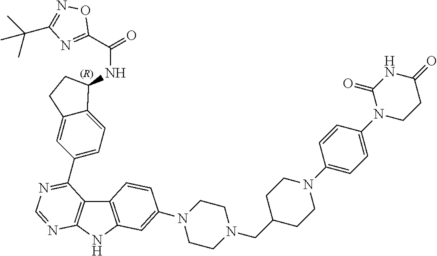

To a solution of (R)-3-(tert-butyl)-N-(5-(7-(piperazin-1-yl)-9H-pyrimido[4,5-b]indol-4-yl)-2,3-dihydro-1H-inden-1-yl)-1,2,4-oxadiazole-5-carboxamide (100 mg, crude) in dichloromethane (5 mL) and MeOH (5 mL), NaOAc (47 mg, 0.558 mmol) was added. The mixture was stirred at room temperature for 5 mins. Then 1-(4-(2,4-dioxotetrahydropyrimidin-1(2H)-yl)phenyl)piperidine-4-carbaldehyde (67.4 mg, 0.223 mmol) and HOAc (0.06 mL) were added. The mixture was stirred at room temperature overnight. Then NaBH(OAc)₃ (197 mg, 0.93 mmol) was added and stirred at room temperature for 2 hours. The reaction mixture was concentrated in vacuum, and the residue was purified by silica gel column chromatography (DCM:MeOH=100%:0%˜90%:10% gradient elution) to give the product (50.27 mg, 32%). ¹H NMR (500 MHz, DMSO) δ 12.17 (s, 1H), 10.25 (s, 1H), 9.86 (d, J=5.0 Hz, 1H), 8.83 (s, 1H), 8.83 (s, 1H), 7.82 (s, 1H), 7.78 (d, J=10.0 Hz, 1H), 7.69 (d, J=10.0 Hz, 1H), 7.50 (d, J=10.0 Hz, 1H), 7.13 (d, J=10.0 Hz, 2H), 6.96-6.90 (m, 4H), 5.65 (q, J=10.0 Hz, 1H), 3.73-3.65 (m, 4H), 3.25 (s, 4H), 3.20-3.12 (m, 1H), 3.04-2.95 (m, 1H), 2.71-2.62 (m, 4H), 2.54 (s, 5H), 2.23 (dd, J=20.0 Hz, 10.0 Hz, 3H), 1.82 (d, J=10.0 Hz, 2H), 1.72 (s, 1H), 1.37 (s, 9H), 1.23 (dd, J=20.0 Hz, 10.0 Hz, 2H); [M+H]⁺=822.6.

### Example 38: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrido[2′,3′:4,5]pyrrolo[2,3-d]pyrimidin-4-yl)-2-methylphenyl)ethyl)-1,2,4-oxadiazole-5-carboxamide

### Step 1: ethyl 2-(5-bromo-3-nitropyridin-2-yl)-2-cyanoacetate

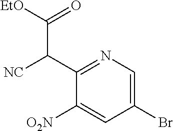 [SMILES: CCOC(=O)C(C#N)c1ncc(Br)cc1[N+](=O)[O-]]

To a stirred solution of ethyl cyanoacetate (24.3 g, 214.790 mmol) in t-BuOH (60 mL) and i-PrOH (100 mL, 0.17 mmol) was added t-BuOK (42.5 g, 227.43 mmol) in portions at room temperature under nitrogen atmosphere. The resulting mixture was stirred for 0.5 h at room temperature under nitrogen atmosphere. Then to the above mixture was added 5-bromo-2-chloro-3-nitropyridine (30 g, 126.35 mmol) in portions at room temperature. The resulting mixture was stirred for additional 7 h at 100° C. and concentrated under reduced pressure. The resulting mixture was diluted with 1 M HCl (100 mL) and extracted with EA (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 1˜30^{%} EA in petroleum ether to afford the product (24 g, 60%). [M−H]⁻=312.0.

### Step 2: ethyl 2-amino-6-bromo-1H-pyrrolo[3,2-b]pyridine-3-carboxylate

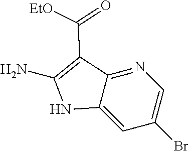 [SMILES: CCOC(=O)c1c(N)[nH]c2cc(Br)cnc12]

To a stirred solution of ethyl 2-(5-bromo-3-nitropyridin-2-yl)-2-cyanoacetate (18 g, 57.31 mmol) in AcOH (200 mL) was added Fe (16 g, 286.54 mmol) in portions at room temperature. The resulting mixture was stirred for 2 h at room temperature. The resulting mixture was filtered, and the filter cake was washed with MeOH (3×50 mL). The filtrate was concentrated under reduced pressure. The resulting mixture was extracted with EA (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 1˜100% EA in PE afford the product (20 g, 92%). [M+H]⁺=284.1.

### Step 3: 7-bromo-3,9-dihydro-4H-pyrido[2′,3′:4,5]pyrrolo[2,3-d]pyrimidin-4-one

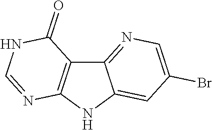 [SMILES: O=c1[nH]cnc2[nH]c3cc(Br)cnc3c12]

A solution of ethyl 2-amino-6-bromo-1H-pyrrolo[3,2-b]pyridine-3-carboxylate (27 g, 95.03 mmol) and formamidine acetate (29.3 g, 281.58 mmol) in 2-methoxyethanol (300 mL) was stirred overnight at 125° C. The mixture was allowed to cool down to room temperature. The resulting mixture was diluted with water (300 mL). The precipitated solid was collected by filtration. The resulting solid was dried under infrared light to afford the product (9 g, 35%). [M+H]⁺=264.9.

### Step 4: tert-butyl 4-(4-oxo-4,9-dihydro-3H-pyrido[2′,3′:4,5]pyrrolo[2,3-d]pyrimidin-7-yl)-3,6-dihydropyridine-1(2H)-carboxylate

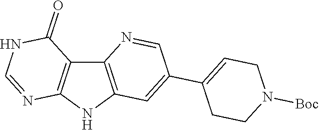 [SMILES: CC(C)(C)OC(=O)N1CC=C(c2cnc3c(c2)[nH]c2nc[nH]c(=O)c23)CC1]

To a stirred solution of 7-bromo-3,9-dihydro-4H-pyrido[2′,3′:4,5]pyrrolo[2,3-d]pyrimidin-4-one (8 g, 30.18 mmol) and tert-butyl 4-(4,4,5,5-tetramethyl-1,3,2-dioxaborolan-2-yl)-3,6-dihydropyridine-1(2H)-carboxylate (13.3 g, 45.26 mmol) in dioxane (120 mL) and H₂O (24 mL) were added K₃PO₄ (12.8 g, 60.36 mmol) and Pd(dppf)Cl₂ (2.2 g, 3.02 mmol). The resulting mixture was stirred overnight at 80° C. under nitrogen atmosphere. The residue was purified by silica gel column chromatography, eluted with CH₂Cl₂/MeOH (5:1, 0.1% TEA) to afford the product (7 g, 63%). [M+H]⁺=368.1.

### Step 5: tert-butyl 4-(4-oxo-4,9-dihydro-3H-pyrido[2′,3′:4,5]pyrrolo[2,3-d]pyrimidin-7-yl)piperidine-1-carboxylate

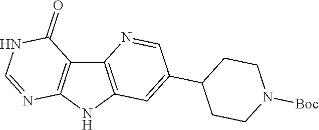 [SMILES: CC(C)(C)OC(=O)N1CCC(c2cnc3c(c2)[nH]c2nc[nH]c(=O)c23)CC1]

To a stirred solution of tert-butyl 4-(4-oxo-4,9-dihydro-3H-pyrido[2′,3′:4,5]pyrrolo[2,3-d]pyrimidin-7-yl)-3,6-dihydropyridine-1(2H)-carboxylate (10.7 g, 29.12 mmol) in DMF (30 mL), EtOH (120 mL) and EA (120 mL) were added Pd/C (5 g) and Pd(OH)₂/C (5.00 g). The resulting mixture was stirred for 16 h at 50° C. under hydrogen atmosphere at 20 atm. The mixture was allowed to cool down to room temperature. The residue was purified by silica gel column chromatography, eluted with DCM/MeOH (5:1) to afford the product (5.7 g, 53%). [M+H]⁺=370.2.

### Step 6: 4-chloro-7-(piperidin-4-yl)-9H-pyrido[2′,3′:4,5]pyrrolo[2,3-d]pyrimidine

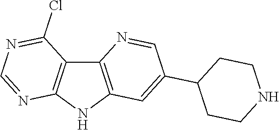 [SMILES: Clc1ncnc2[nH]c3cc(C4CCNCC4)cnc3c12]

A solution of tert-butyl 4-(4-oxo-4,9-dihydro-3H-pyrido[2′,3′:4,5]pyrrolo[2,3-d]pyrimidin-7-yl)piperidine-1-carboxylate (1.60 g, 4.33 mmol) in POCl₃ (30.00 mL, 321.84 mmol) was stirred for 2 h at 110° C. The resulting mixture was concentrated under vacuum. The residue was dissolved in methylbenzene (3×15 mL). The resulting mixture was concentrated under vacuum. The crude product was used for the next step without further purification. [M+H]⁺=288.1.

### Step 7: tert-butyl 4-(4-chloro-9H-pyrido[2′,3′:4,5]pyrrolo[2,3-d]pyrimidin-7-yl)piperidine-1-carboxylate

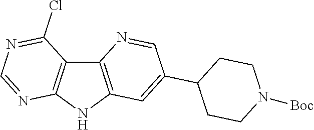 [SMILES: CC(C)(C)OC(=O)N1CCC(c2cnc3c(c2)[nH]c2ncnc(Cl)c23)CC1]

A solution of 4-chloro-7-(piperidin-4-yl)-9H-pyrido[2′,3′:4,5]pyrrolo[2,3-d]pyrimidine (1.8 g, 6.26 mmol) in DMF (50 mL, 646.09 mmol) was stirred for 5 min at room temperature. The mixture was neutralized to pH 7 with saturated NaHCO₃ (aq.). To the above mixture was added Boc₂O (13.6 g, 62.54 mmol) at room temperature. The resulting mixture was stirred for additional 16 h at room temperature. The resulting mixture was extracted with EA (2×200 mL). The combined organic layers were washed with brine (2×100 mL) and dried over anhydrous Na₂SO₄. After filtration, the filtrate was concentrated under reduced pressure. The residue was purified by silica gel column chromatography, eluted with PE/EA (1:1) to afford the product (1 g, 41%). [M+H]⁺=388.1.

### Step 8: tert-butyl 7-(1-(tert-butoxycarbonyl)piperidin-4-yl)-4-chloro-9H-pyrido[2′,3′:4,5]pyrrolo[2,3-d]pyrimidine-9-carboxylate

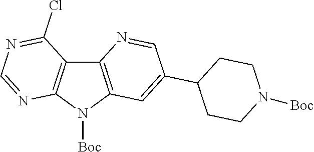 [SMILES: CC(C)(C)OC(=O)N1CCC(c2cnc3c4c(Cl)ncnc4n(C(=O)OC(C)(C)C)c3c2)CC1]

To a stirred solution of tert-butyl 4-(4-chloro-9H-pyrido[2′,3′:4,5]pyrrolo[2,3-d]pyrimidin-7-yl)piperidine-1-carboxylate (1 g, 2.58 mmol) and DIEA (2.94 mL, 16.88 mmol) in DCM (30 mL) were added DMF (9 mL) and Boc₂O (1 g, 5.18 mmol) at room temperature. The resulting mixture was stirred for 16 h at room temperature and concentrated under vacuum. The residue was purified by silica gel column chromatography, eluted with PE/EA (3:1) afford the product (166 mg, 13%). [M+H]⁺=488.2.

### Step 9: tert-butyl (R)-7-(1-(tert-butoxycarbonyl)piperidin-4-yl)-4-(4-(1-(3-(tert-butyl)-1,2,4-oxadiazole-5-carboxamido)ethyl)-3-methylphenyl)-9H-pyrido[2′,3′:4,5]pyrrolo[2,3-d]pyrimidine-9-carboxylate

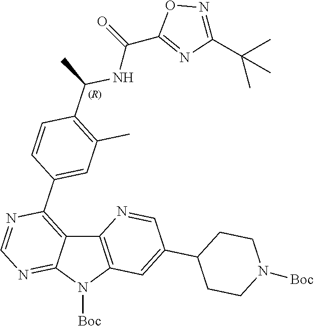

To a stirred solution of tert-butyl 7-(1-(tert-butoxycarbonyl)piperidin-4-yl)-4-chloro-9H-pyrido[2′,3′:4,5]pyrrolo[2,3-d]pyrimidine-9-carboxylate (166 mg, 0.34 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (169 mg, 0.41 mmol) in dioxane (5 mL) and H₂O (1 mL) were added K₃PO₄ (144 mg, 0.68 mmol) and Pd(dppf)Cl₂ (25 mg, 0.034 mmol) at room temperature. The resulting mixture was stirred for 2 h at 80° C. under nitrogen atmosphere and concentrated under vacuum. The residue was purified by silica gel column chromatography, eluted with CH₂Cl₂/EA (3:1) to afford the product (87 mg, 34%). [M+H]⁺=739.5.

### Step 10: (R)-3-(tert-butyl)-N-(1-(2-methyl-4-(7-(piperidin-4-yl)-9H-pyrido[2′,3′:4,5]pyrrolo[2,3-d]pyrimidin-4-yl)phenyl)ethyl)-1,2,4-oxadiazole-5-carboxamide

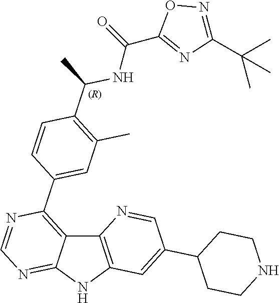

To a stirred solution of tert-butyl (R)-7-(1-(tert-butoxycarbonyl)piperidin-4-yl)-4-(4-(1-(3-(tert-butyl)-1,2,4-oxadiazole-5-carboxamido)ethyl)-3-methylphenyl)-9H-pyrido[2′,3′:4,5]pyrrolo[2,3-d]pyrimidine-9-carboxylate (87 mg, 0.12 mmol) in DCM (5 mL) was added TFA (1 mL) dropwise at room temperature. The resulting mixture was stirred for 1 h at room temperature and concentrated under vacuum. The residue was purified by reverse phase flash chromatography with the following conditions: Column: Spherical C18, 20-40 um, 40 g; Mobile Phase A: Water (plus 5 mM FA); Mobile Phase B: ACN; Flow rate: 80 mL/min; Gradient: 5%-5% B, 10 min, 10% B-30% B gradient in 30 min; Detector: 220 nm. The fractions containing the desired product were collected at 29% B and concentrated under reduced pressure to afford the product (31 mg, 49%). ¹H NMR (400 MHz, DMSO) δ 9.95 (d, J=8.0 Hz, 1H), 9.04 (s, 1H), 8.61 (d, J=8.4 Hz, 1H), 8.53 (d, J=2.0 Hz, 1H), 8.47 (d, J=2.0 Hz, 1H), 8.36 (s, 1H), 7.78 (d, J=2.0 Hz, 1H), 7.69 (d, J=8.4 Hz, 1H), 5.42 (t, J=7.2 Hz, 1H), 3.22 (d, J=12.0 Hz, 2H), 2.99 (t, J=12.0 Hz, 1H), 2.81 (t, J=12.0 Hz, 2H), 2.53 (s, 3H), 1.91 (d, J=12.4 Hz, 2H), 1.78 (d, J=12.4 Hz, 2H), 1.57 (d, J=6.8 Hz, 3H), 1.38 (s, 9H), 1.24 (s, 1H); [M+H]⁺=538.9.

### Step 12: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrido[2′,3′:4,5]pyrrolo[2,3-d]pyrimidin-4-yl)-2-methylphenyl)ethyl)-1,2,4-oxadiazole-5-carboxamide

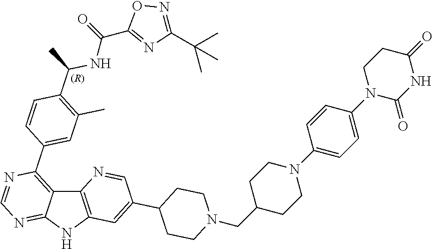

To a mixture of (R)-3-(tert-butyl)-N-(1-(2-methyl-4-(7-(piperidin-4-yl)-9H-pyrido[2′,3′:4,5]pyrrolo[2,3-d]pyrimidin-4-yl)phenyl)ethyl)-1,2,4-oxadiazole-5-carboxamide (7 mg, 0.013 mmol) in dichloromethane (10 mL) and MeOH (2 mL) was added 1-(4-(2,4-dioxotetrahydropyrimidin-1(2H)-yl)phenyl)piperidine-4-carbaldehyde (7 mg, 0.023 mmol). Then the mixture was stirred in a round bottom flask at room temperature for 1 hour. To the mixture was added NaBH(OAc)₃ (14 mg, 0.065 mmol) and stirred at room temperature overnight. Then the mixture was evaporated in vacuum to afford the crude product, which was purified with Pre-TLC (DCM:MeOH=9:1) to give the product (2 mg, 18%). ¹H NMR (400 MHz, DMSO) δ 12.79 (s, 1H), 11.06-10.36 (m, 1H), 10.27 (s, 1H), 9.95 (d, J=7.6 Hz, 1H), 9.04 (s, 1H), 8.69-8.51 (m, 2H), 8.46 (s, 1H), 7.87 (s, 1H), 7.70 (d, J=8.0 Hz, 1H), 7.15 (d, J=8.8 Hz, 2H), 6.96 (d, J=8.4 Hz, 2H), 5.53-5.28 (m, 1H), 3.78-3.60 (m, 5H), 3.42-3.35 (m, 1H), 3.19-2.95 (m, 4H), 2.77-2.64 (m, 4H), 2.58-2.51 (m, 6H), 2.13-1.95 (m, 4H), 1.57 (d, J=6.8 Hz, 3H), 1.43-1.31 (m, 9H), 1.23 (s, 2H); [M+H]⁺=824.7.

### Example 39: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrido[3′,2′:4,5]pyrrolo[2,3-d]pyrimidin-4-yl)-2-methylphenyl)ethyl)-1,2,4-oxadiazole-5-carboxamide

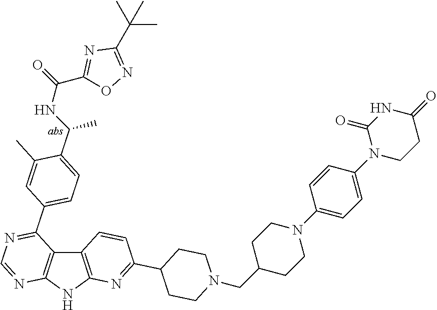 [SMILES: Cc1cc(-c2ncnc3[nH]c4nc(C5CCN(CC6CCN(c7ccc(N8CCC(=O)NC8=O)cc7)CC6)CC5)ccc4c23)ccc1[C@@H](C)NC(=O)c1nc(C(C)(C)C)no1]

The titled compound was synthesized in the procedures similar to Example 38. ¹H NMR (400 MHz, DMSO) δ 12.93 (s, 1H), 10.25 (s, 1H), 9.94 (d, J=7.6 Hz, 1H), 8.14 (d, J=7.6 Hz, 1H), 7.84 (d, J=7.6 Hz, 1H), 7.79 (s, 1H), 7.73 (d, J=7.6 Hz, 1H), 7.23 (d, J=8.0 Hz, 1H), 7.13 (d, J=8.4 Hz, 2H), 6.93 (d, J=8.4 Hz, 2H), 5.49-5.37 (m, 1H), 3.76-3.62 (m, 4H), 3.00-2.92 (m, 2H), 2.79 (brs, 1H), 2.73-2.60 (m, 4H), 2.26-2.16 (m, 2H), 2.09-1.96 (m, 2H), 1.94-1.76 (m, 6H), 1.70 (brs, 1H), 1.58 (d, J=6.8 Hz, 3H), 1.37 (s, 9H), 1.27-1.20 (m, 2H); [M+H]⁺=824.7.

### Example 40: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-5-fluoro-2-(hydroxymethyl)phenyl)ethyl)-1,2,4-oxadiazole-5-carboxamide

### Step 1: benzyl (R)-4-(4-(4-(1-(3-(tert-butyl)-1,2,4-oxadiazole-5-carboxamido)ethyl)-5-(((tert-butyldiphenylsilyl)oxy)methyl)-2-fluorophenyl)-6-methyl-9H-pyrimido[4,5-b]indol-7-yl)piperazine-1-carboxylate

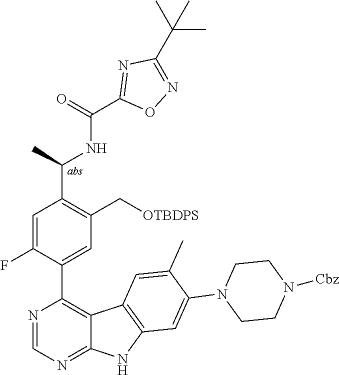

A mixture of benzyl 4-(4-chloro-6-methyl-9H-pyrimido[4,5-b]indol-7-yl)piperazine-1-carboxylate (169.3 mg, 0.39 mmol), (R)-3-(tert-butyl)-N-(1-(2-(((tert-butyldiphenylsilyl)oxy)methyl)-5-fluoro-4-(4,4,5,5-tetramethyl-1,3,2-dioxaborolan-2-yl)phenyl)ethyl)-1,2,4-oxadiazole-5-carboxamide (the compound was obtained through the same way in WO2021219070A) (320 mg, 0.47 mmol), Pd(PPh₃)₄ (45 mg, 0.04 mmol) and K₂CO₃ (107.4 mg, 0.78 mmol) in dioxane (10 mL) and H₂O (2 mL) was stirred at 95° C. for 16 h under a nitrogen atmopshere. The mixture was concentrated under vacuum. The residue was purified by silica gel column chromatography, eluted with EA/PE (2:1) to afford the product (260 mg, 69.7%). [M+H]⁺=959.5.

### Step 2: (R)-3-(tert-butyl)-N-(1-(5-fluoro-2-(hydroxymethyl)-4-(6-methyl-7-(piperazin-1-yl)-9H-pyrimido[4,5-b]indol-4-yl)phenyl)ethyl)-1,2,4-oxadiazole-5-carboxamide trifluoroacetate

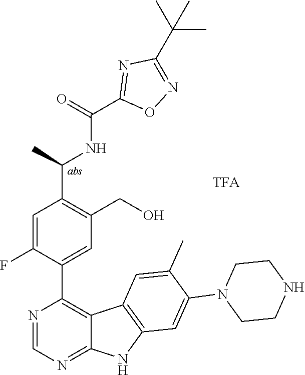 [SMILES: Cc1cc2c(cc1N1CCNCC1)[nH]c1ncnc(-c3cc(CO)c([C@@H](C)NC(=O)c4nc(C(C)(C)C)no4)cc3F)c12]

A mixture of benzyl (R)-4-(4-(4-(1-(3-(tert-butyl)-1,2,4-oxadiazole-5-carboxamido)ethyl)-5-(((tert-butyldiphenylsilyl)oxy)methyl)-2-fluorophenyl)-6-methyl-9H-pyrimido[4,5-b]indol-7-yl)piperazine-1-carboxylate (100 mg, 0.104 mmol) in TFA (5 mL) was refluxed for 2 h. The mixture was concentrated under vacuum to afford the product (90 mg, crude), which was used in the next step directly. [M+H]⁺=587.3.

### Step 3: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-5-fluoro-2-(hydroxymethyl)phenyl)ethyl)-1,2,4-oxadiazole-5-carboxamide

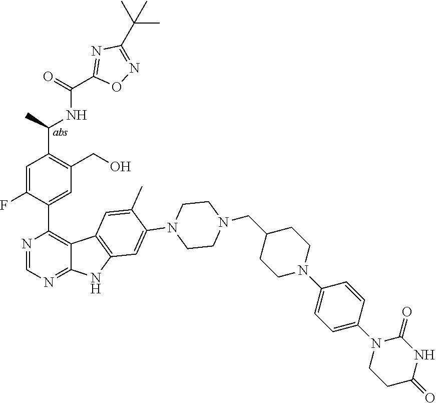 [SMILES: Cc1cc2c(cc1N1CCN(CC3CCN(c4ccc(N5CCC(=O)NC5=O)cc4)CC3)CC1)[nH]c1ncnc(-c3cc(CO)c([C@@H](C)NC(=O)c4nc(C(C)(C)C)no4)cc3F)c12]

A mixture of (R)-3-(tert-butyl)-N-(1-(5-fluoro-2-(hydroxymethyl)-4-(6-methyl-7-(piperazin-1-yl)-9H-pyrimido[4,5-b]indol-4-yl)phenyl)ethyl)-1,2,4-oxadiazole-5-carboxamide trifluoroacetate (90 mg, 0.15 mmol), 1-(4-(2,4-dioxotetrahydropyrimidin-1(2H)-yl)phenyl)piperidine-4-carbaldehyde (91 mg, 0.20 mmol) and AcOH (0.02 mL) in MeOH (5 mL) and DCM (5 mL) was stirred at rt for 16 h. Then, STAB (63.6 mg, 0.3 mmol) was added to the mixture above. The mixture was stirred at rt for 5 h. The mixture was diluted with water (50 mL) and extracted with DCM (3×100 mL). The combined organic layers were dried over Na₂SO₄, filtered and concentrated under vacuum. The residue was purified by silica gel column chromatography, eluted with MeOH/DCM (1:9) to afford the product (59.48 mg, 44.4%). ¹H NMR (400 MHz, DMSO) δ 12.30 (s, 1H), 10.26 (s, 1H), 9.93 (d, J=8.0 Hz, 1H), 8.91 (s, 1H), 7.75 (d, J=8.0 Hz, 1H), 7.64 (d, J=12.0 Hz, 1H), 7.23-7.10 (m, 4H), 6.93 (d, J=8.0 Hz, 2H), 5.51-5.42 (m, 1H), 5.41-5.35 (m, 1H), 4.88-4.78 (m, 1H), 4.76-4.65 (m, 1H), 3.75-3.65 (m, 4H), 3.00-2.88 (m, 4H), 2.74-2.61 (m, 4H), 2.61-2.53 (m, 3H), 2.30-2.23 (m, 2H), 2.20 (s, 3H), 1.86-1.78 (m, 2H), 1.76-1.67 (m, 1H), 1.61 (d, J=4.0 Hz, 3H), 1.37 (s, 9H), 1.31-1.18 (m, 3H); [M+H]⁺=872.4.

### Example 41: 3-(tert-butyl)-N-((1R)-1-(4-(7-(1-(3-(4-(2,6-dioxopiperidin-3-yl)phenyl)cyclobutyl)piperidin-4-yl)-9H-pyrido[3′,4′:4,5]pyrrolo[2,3-d]pyrimidin-4-yl)-2-methylphenyl)ethyl)-1,2,4-oxadiazole-5-carboxamide

### Step 1: 3-(4-(2,6-bis(benzyloxy)pyridin-3-yl)phenyl)cyclobutan-1-one

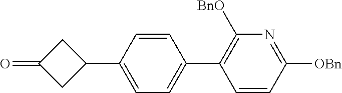 [SMILES: O=C1CC(c2ccc(-c3ccc(OCc4ccccc4)nc3OCc3ccccc3)cc2)C1]

To a solution of 3-(4-bromophenyl)cyclobutan-1-one (4.0 g, 17.7 mmol) in dioxane/H₂O (50 mL) were added 2,6-bis(benzyloxy)-3-(4,4,5,5-tetramethyl-1,3,2-dioxaborolan-2-yl)pyridine (7.4 g, 17.7 mmol), K₂CO₃ (7.3 g, 53 mmol) and Pd(dppf)Cl₂.DCM (1.4 g, 1.77 mmol). The reaction was stirred at 100° C. for 6 hours. The solvent was evaporated and extracted with EA (50 mL*2). The organic phase was combined and purified by flash chromatography with PE/EA (100:1 to 3:1) to give the product (3.2 g, 41.6%). ¹H NMR (400 MHz, CDCl₃) δ 7.60 (d, J=8.0 Hz, 1H), 7.56 (d, J=8.0 Hz, 2H), 7.48-7.20 (m, 12H), 6.48 (d, J=8.0 Hz, 1H), 5.44 (s, 2H), 5.37 (s, 2H), 3.78-3.64 (m, 1H), 3.55-3.48 (m, 2H), 3.32-3.26 (m, 2H).

### Step 2: 3-(4-(3-oxocyclobutyl)phenyl)piperidine-2,6-dione

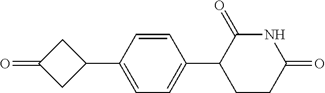 [SMILES: O=C1CC(c2ccc(C3CCC(=O)NC3=O)cc2)C1]

To a solution of 3-(4-(2,6-bis(benzyloxy)pyridin-3-yl)phenyl)cyclobutan-1-one (0.5 g, 1.1 mmol) in THF (30 mL) was added Pd/C (50 mg). The reaction mixture was stirred at 60° C. under H₂ (1 atm) for 18 hours. After filtration, the filtrate was concentrated and used for next step directly.

### Step 3: 3-(tert-butyl)-N-((1R)-1-(4-(7-(1-(3-(4-(2,6-dioxopiperidin-3-yl)phenyl)cyclobutyl)piperidin-4-yl)-9H-pyrido[3′,4′:4,5]pyrrolo[2,3-d]pyrimidin-4-yl)-2-methylphenyl)ethyl)-1,2,4-oxadiazole-5-carboxamide

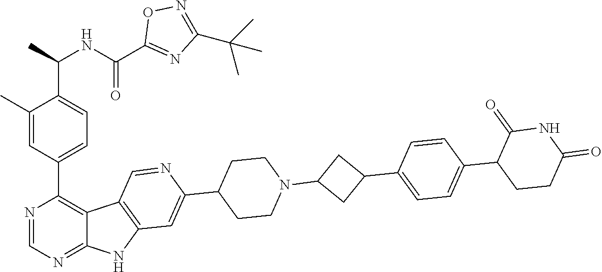 [SMILES: Cc1cc(-c2ncnc3[nH]c4cc(C5CCN(C6CC(c7ccc(C8CCC(=O)NC8=O)cc7)C6)CC5)ncc4c23)ccc1[C@@H](C)NC(=O)c1nc(C(C)(C)C)no1]

To a solution of (R)-3-(tert-butyl)-N-(1-(2-methyl-4-(7-(piperidin-4-yl)-9H-pyrido[3′,4′:4,5]pyrrolo[2,3-d]pyrimidin-4-yl)phenyl)ethyl)-1,2,4-oxadiazole-5-carboxamide (the compound was obtained through the similar way in example 38) (160 mg, 0.3 mmol) and 3-(4-(3-oxocyclobutyl)phenyl)piperidine-2,6-dione (77 mg, 0.3 mmol) in DCM/EtOH (20 mL) was added NaOAc (74 mg, 0.9 mmol). After stirring for 3 hours, NaBH(OAc)₃ (191 mg, 0.9 mmol) was added. The reaction mixture was stirred for another 3 hours. The solvent was evaporated and purified by pre-TLC with DCM/MeOH (10:1) to give the product (48.7 mg, 20.8%). ¹H NMR (400 MHz, DMSO) δ 11.22 (brs, 1H), 10.83 (s, 1H), 9.99 (d, J=8.0 Hz, 1H), 9.17 (s, 1H), 9.00 (s, 1H), 7.99-7.82 (m, 2H), 7.77 (d, J=8.0 Hz, 1H), 7.67 (brs, 1H), 7.42 (d, J=8.4 Hz, 2H), 7.20 (t, J=7.6 Hz, 2H), 5.51-5.29 (m, 1H), 3.85-3.83 (m, 1H), 3.66-3.17 (m, 18H), 2.66 (d, J=10.74 Hz, 3H), 2.55 (s, 3H), 2.50 (s, 3H), 2.22 (brs, 5H), 2.04 (brs, 1H), 1.59 (d, J=6.4 Hz, 3H), 1.37 (s, 9H); [M+H]⁺=780.7.

### Example 42: (R)-3-(tert-butyl)-N-(1-(4-(7-(1-((1-(4-(2,4-dioxotetrahydropyrimidin-1(2H)-yl)phenyl)piperidin-4-yl)methyl)piperidin-4-yl)-9H-pyrido[3′,4′:4,5]pyrrolo[2,3-d]pyrimidin-4-yl)-2-methylphenyl)ethyl)-1,2,4-oxadiazole-5-carboxamide

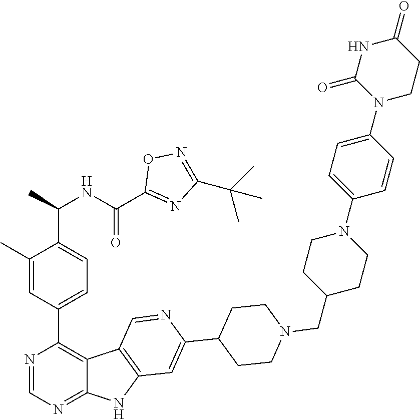 [SMILES: Cc1cc(-c2ncnc3[nH]c4cc(C5CCN(CC6CCN(c7ccc(N8CCC(=O)NC8=O)cc7)CC6)CC5)ncc4c23)ccc1[C@@H](C)NC(=O)c1nc(C(C)(C)C)no1]

To a solution of (R)-3-(tert-butyl)-N-(1-(2-methyl-4-(7-(piperidin-4-yl)-9H-pyrido[3′,4′:4,5]pyrrolo[2,3-d]pyrimidin-4-yl)phenyl)ethyl)-1,2,4-oxadiazole-5-carboxamide (90 mg, 0.17 mmol) and 1-(4-(2,4-dioxotetrahydropyrimidin-1(2H)-yl) phenyl)piperidine-4-carbaldehyde (50 mg, 0.17 mmol) in DCM/EtOH (30 mL) was added NaOAc (42 mg, 0.51 mmol). After stirring for 0.5 hour, NaBH(OAc)₃ (108 mg, 0.51 mmol) was added. The reaction mixture was stirred for another 18 hours. The solvent was evaporated and purified by pre-TLC with DCM/MeOH (10:1) to give the product (85.2 mg, 60.8%). ¹H NMR (400 MHz, DMSO) δ 10.30 (s, 1H), 9.99 (d, J=7.2 Hz, 2H), 9.17 (s, 1H), 9.00 (s, 1H), 8.01-7.82 (m, 2H), 7.77 (d, J=8.0 Hz, 1H), 7.66 (brs, 1H), 7.30-6.80 (m, 4H), 5.50-5.31 (m, 1H), 3.81-3.65 (m, 8H), 3.25-3.08 (m, 5H), 2.69 (t, J=6.8 Hz, 2H), 2.55 (s, 3H), 2.33 (brs, 3H), 2.23-1.89 (m, 6H), 1.59 (d, J=6.4 Hz, 3H), 1.37 (s, 9H); [M+H]⁺=824.7.

### Example 43: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-2-methylphenyl)ethyl)-1,2,4-oxadiazole-5-carboxamide

### Step 1: benzyl 4-(4-chloro-2-methylphenyl)piperazine-1-carboxylate

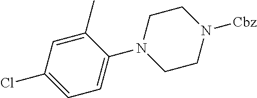 [SMILES: Cc1cc(Cl)ccc1N1CCN(C(=O)OCc2ccccc2)CC1]

To a stirred mixture of 1-bromo-4-chloro-2-methylbenzene (50 g, 243.3 mmol) and benzyl piperazine-1-carboxylate (64.3 g, 292 mmol) in toluene (1 L) were added t-BuONa (46.77 g, 486.6 mmol), BINAP (30.3 g, 48.7 mmol) and Pd₂(dba)₃ (22.3 g, 24.3 mmol) at room temperature under nitrogen atmosphere. The resulting mixture was stirred for 2 h at 95° C. under nitrogen atmosphere. The reaction was quenched with water at room temperature. The resulting mixture was extracted with EtOAc (3×1 L). The combined organic layers were washed with brine (2×800 mL) and dried over anhydrous Na₂SO₄. After filtration, the filtrate was concentrated under reduced pressure. The residue was purified by silica gel column chromatography, eluted with EtOAc in PE (0-30%) to afford the product (51.3 g, 61.1%). [M+H]⁺=345.1.

### Step 2: 1-(4-chloro-2-methyl-5-nitrophenyl)piperazine

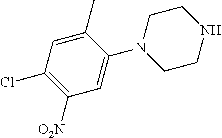 [SMILES: Cc1cc(Cl)c([N+](=O)[O-])cc1N1CCNCC1]

To a stirred solution of benzyl 4-(4-chloro-2-methylphenyl)piperazine-1-carboxylate (15 g, 43.5 mmol) in conc. H₂SO₄ (60 mL) was added conc. HNO₃ (27.6 mL) dropwise at 0° C. under nitrogen atmosphere. The resulting mixture was stirred for 0.5 h at 0° C. The reaction was quenched with Na₂CO₃ at 0° C. The resulting mixture was extracted with EtOAc (3×1 L). The combined organic layers were washed with brine (2×800 mL) and dried over anhydrous Na₂SO₄. After filtration, the filtrate was concentrated under reduced pressure to afford the product (26.5 g, crude). [M+H]⁺=256.2.

### Step 3: benzyl 4-(4-chloro-2-methyl-5-nitrophenyl)piperazine-1-carboxylate

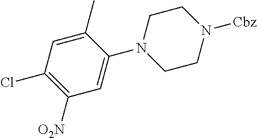 [SMILES: Cc1cc(Cl)c([N+](=O)[O-])cc1N1CCN(C(=O)OCc2ccccc2)CC1]

To a stirred solution of 1-(4-chloro-2-methyl-5-nitrophenyl)piperazine (26.5 g, 103.6 mmol) in DCM (280 mL) were added Cbz-Cl (35.4 g, 207.3 mmol) and DIEA (72.2 mL, 558.7 mmol) at room temperature. The resulting mixture was stirred for 16 h at room temperature and concentrated under reduced pressure. The residue was purified by silica gel column chromatography, eluted with EtOAc in PE (0-30%) to afford the product (36.6 g, 57.8%). [M+H]⁺=391.1.

### Step 4: benzyl 4-(4-(1-cyano-2-ethoxy-2-oxoethyl)-2-methyl-5-nitrophenyl)piperazine-1-carboxylate

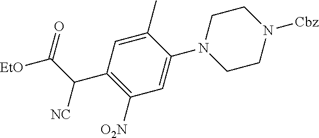 [SMILES: CCOC(=O)C(C#N)c1cc(C)c(N2CCN(C(=O)OCc3ccccc3)CC2)cc1[N+](=O)[O-]]

To a stirred mixture of Cs₂CO₃ (91.8 g, 281.6 mmol) in DMF (200 mL) was added ethyl cyanoacetate (15.0 mL, 132.5 mmol) in DMF (100 mL) dropwise at room temperature under nitrogen atmosphere. The resulting mixture was stirred for 0.5 h at room temperature under nitrogen atmosphere. To the above mixture was added benzyl 4-(4-chloro-2-methyl-5-nitrophenyl)piperazine-1-carboxylate (36.6 g, 93.9 mmol) in DMF (100 mL) at 0° C. The resulting mixture was stirred for 16 h at 70° C. The mixture was allowed to cool down to room temperature. The mixture was acidified to pH=6 with HCl (aq.) (1 M). The resulting mixture was extracted with EtOAc (3×800 mL). The combined organic layers were washed with brine (3×300 mL) and dried over anhydrous Na₂SO₄. After filtration, the filtrate was concentrated under reduced pressure. The residue was purified by silica gel column chromatography, eluted with EtOAc in PE (0-30%) to afford the product (52.3 g, crude). [M+H]⁺=467.3.

### Step 5: ethyl 2-amino-6-(4-((benzyloxy)carbonyl)piperazin-1-yl)-5-methyl-1H-indole-3-carboxylate

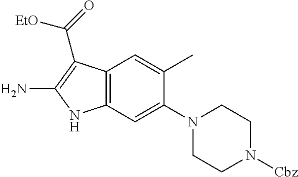 [SMILES: CCOC(=O)c1c(N)[nH]c2cc(N3CCN(C(=O)OCc4ccccc4)CC3)c(C)cc12]

A mixture of benzyl 4-(4-(1-cyano-2-ethoxy-2-oxoethyl)-2-methyl-5-nitrophenyl)piperazine-1-carboxylate (52.3 g, 112.1 mmol), NH₄Cl (18.0 g, 336.4 mmol) and Fe (62.6 g, 1121.1 mmol) in EtOH (600 mL) and H₂O (12 mL) was stirred for 32 h at 80° C. The mixture was allowed to cool down to room temperature and filtered. The filtrate was concentrated under reduced pressure. The residue was purified by silica gel column chromatography, eluted with EtOAc in PE (1%-30%) to afford the product (6.8 g, 13.9%). [M+H]⁺=437.1.

### Step 6: benzyl 4-(6-methyl-4-oxo-4,9-dihydro-3H-pyrimido[4,5-b]indol-7-yl)piperazine-1-carboxylate

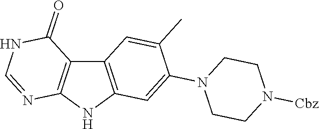 [SMILES: Cc1cc2c(cc1N1CCN(C(=O)OCc3ccccc3)CC1)[nH]c1nc[nH]c(=O)c12]

A mixture of ethyl 2-amino-6-(4-((benzyloxy)carbonyl)piperazin-1-yl)-5-methyl-1H-indole-3-carboxylate (6.8 g, 15.6 mmol) and formamidine acetate (16.2 g, 155.8 mmol) in 2-methoxyethan-1-ol (68 mL) was stirred for 24 h at 125° C. The mixture was allowed to cool down to room temperature. To the resulting mixture was added water and filtered. The filter cake was washed with water (3×200 mL). The solid was dried under infrared light to give the product (5.4 g, 83%). [M+H]⁺=418.2.

### Step 7: benzyl 4-(4-chloro-6-methyl-9H-pyrimido[4,5-b]indol-7-yl)piperazine-1-carboxylate

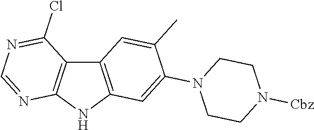 [SMILES: Cc1cc2c(cc1N1CCN(C(=O)OCc3ccccc3)CC1)[nH]c1ncnc(Cl)c12]

A solution of benzyl 4-(6-methyl-4-oxo-4,9-dihydro-3H-pyrimido[4,5-b]indol-7-yl)piperazine-1-carboxylate (3.4 g, 8.2 mmol) in POCl₃ (7.6 mL, 49.5 mmol) and dioxane (34 mL) was stirred for 2 h at 90° C. The resulting mixture was concentrated under reduced pressure and extracted with EtOAc (3×300 mL). The combined organic layers were washed with brine (2×80 mL) and dried over anhydrous Na₂SO₄. After filtration, the filtrate was concentrated under reduced pressure. The residue was purified by silica gel column chromatography, eluted with MeOH in DCM (0-5%) to afford the product (1.5 g, 42.2%). [M+H]⁺=436.2.

### Step 8: benzyl(R)-4-(4-(4-(1-(3-(tert-butyl)-1,2,4-oxadiazole-5-carboxamido)ethyl)-3-methylphenyl)-6-methyl-9H-pyrimido[4,5-b]indol-7-yl)piperazine-1-carboxylate

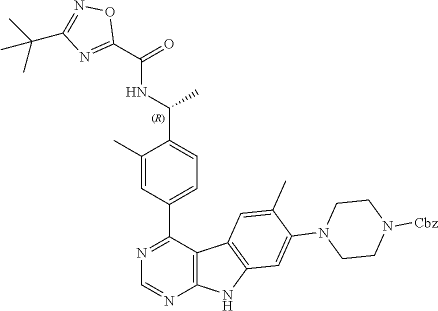

To a stirred mixture of 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (2.3 g, 5.5 mmol) and benzyl 4-(4-chloro-6-methyl-9H-pyrimido[4,5-b]indol-7-yl)piperazine-1-carboxylate (2 g, 4.6 mmol) in DMF (40 mL) and H₂O (8 mL) were added Pd(dppf)Cl₂ (335 mg, 0.46 mmol) and K₃PO₄ (1.95 g, 9.2 mmol) at room temperature under nitrogen atmosphere. The resulting mixture was stirred for 16 h at 80° C. under nitrogen atmosphere. The mixture was allowed to cool down to room temperature and extracted with EtOAc (3×300 mL). The combined organic layers were washed with brine (2×80 mL) and dried over anhydrous Na₂SO₄. After filtration, the filtrate was concentrated under reduced pressure. The residue was purified by silica gel column chromatography, eluted with EtOAc in PE (20%-90%) to afford the product. [M+H]⁺=687.4

### Step 9: (R)-3-(tert-butyl)-N-(1-(2-methyl-4-(6-methyl-7-(piperazin-1-yl)-9H-pyrimido[4,5-b]indol-4-yl)phenyl)ethyl)-1,2,4-oxadiazole-5-carboxamide

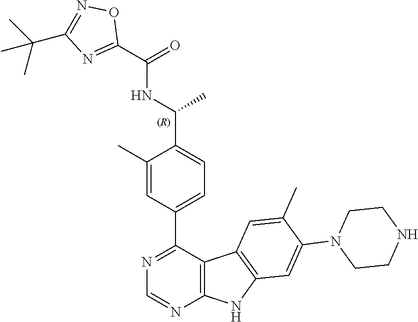

A solution of benzyl (R)-4-(4-(4-(1-(3-(tert-butyl)-1,2,4-oxadiazole-5-carboxamido)ethyl)-3-methylphenyl)-6-methyl-9H-pyrimido[4,5-b]indol-7-yl)piperazine-1-carboxylate (1 g, 1.5 mmol) in TFA (100 mL) was refluxed for 16 h under nitrogen atmosphere. The resulting mixture was concentrated under reduced pressure. The residue was purified by reverse phase flash chromatography with the following conditions: column, C18; mobile phase, MeCN in water (0.1% FA), 5% to 40% gradient in 20 min; detector, UV 254 nm to afford the product (1.4 g, 99.27%). [M+H]⁺=553.3.

### Step 10: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-2-methylphenyl)ethyl)-1,2,4-oxadiazole-5-carboxamide

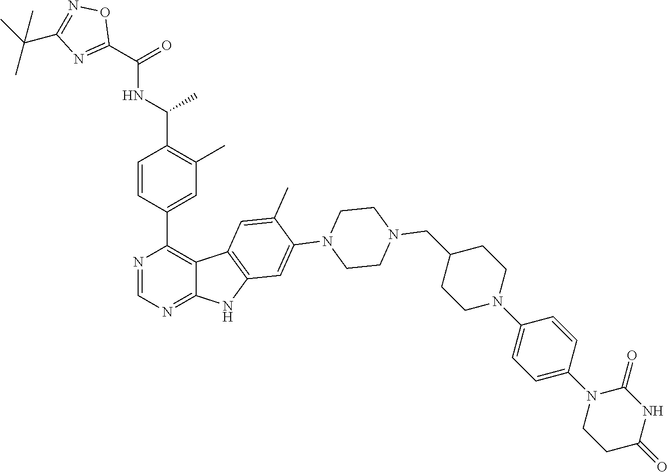 [SMILES: Cc1cc(-c2ncnc3[nH]c4cc(N5CCN(CC6CCN(c7ccc(N8CCC(=O)NC8=O)cc7)CC6)CC5)c(C)cc4c23)ccc1[C@@H](C)NC(=O)c1nc(C(C)(C)C)no1]

A mixture of (R)-3-(tert-butyl)-N-(1-(2-methyl-4-(6-methyl-7-(piperazin-1-yl)-9H-pyrimido[4,5-b]indol-4-yl)phenyl)ethyl)-1,2,4-oxadiazole-5-carboxamide (200 mg, 0.36 mmol), 1-(4-(2,4-dioxotetrahydropyrimidin-1(2H)-yl)phenyl)piperidine-4-carbaldehyde (120 mg, 0.39 mmol) and AcOH (0.02 mL) in MeOH (10 mL) and DCM (10 mL) was stirred at rt overnight. Then, STAB (154 mg, 0.72 mmol) was added to the mixture above and stirred at rt for 5 h. The mixture was diluted with water (100 mL) and extracted with DCM (3×100 mL). The combined organic layers were dried over Na₂SO₄, filtered and concentrated under vacuum. The residue was purified by silica gel column chromatography, eluted with MeOH/DCM (1:7) to afford the product (164.9 mg, 54.3%). ¹H NMR (400 MHz, DMSO) δ 12.24 (s, 1H), 10.26 (s, 1H), 9.43 (d, J=8.0 Hz, 1H), 8.86 (s, 1H), 7.84-7.69 (m, 3H), 7.65 (s, 1H), 7.18-7.09 (m, 3H), 6.93 (d, J=12.0 Hz, 2H), 5.48-5.39 (m, 1H), 3.75-3.64 (m, 4H), 3.03-2.87 (m, 4H), 2.76-2.61 (m, 5H), 2.60-2.54 (m, 3H), 2.52 (s, 3H), 2.26 (s, 5H), 1.83 (d, J=8.0 Hz, 2H), 1.77-1.66 (m, 1H), 1.59 (d, J=8.0 Hz, 3H), 1.37 (s, 9H), 1.31-1.19 (m, 2H); [M+H]⁺=838.4.

### Example 44: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-5-fluoro-2-methylphenyl)ethyl)-1,2,4-oxadiazole-3-carboxamide

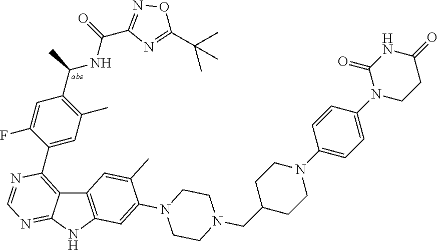 [SMILES: Cc1cc(-c2ncnc3[nH]c4cc(N5CCN(CC6CCN(c7ccc(N8CCC(=O)NC8=O)cc7)CC6)CC5)c(C)cc4c23)c(F)cc1[C@@H](C)NC(=O)c1noc(C(C)(C)C)n1]

The titled compound was synthesized in the procedures similar to Example 43. ¹H NMR (400 MHz, DMSO) δ 12.30 (s, 1H), 11.97 (s, 1H), 10.26 (s, 1H), 9.54 (d, J=8.0 Hz, 1H), 8.90 (s, 1H), 7.62-7.46 (m, 2H), 7.25-7.09 (m, 4H), 6.93 (d, J=8.0 Hz, 1H), 5.48-5.34 (m, 1H), 3.82-3.64 (m, 4H), 3.02-2.87 (m, 4H), 2.75-2.61 (m, 5H), 2.61-2.53 (m, 3H), 2.47 (s, 3H), 2.31-2.17 (m, 5H), 1.87-1.79 (m, 2H), 1.77-1.67 (m, 1H), 1.58 (d, J=4.0 Hz, 3H), 1.44 (s, 9H), 1.30-1.19 (m, 2H); [M+H]⁺=856.4.

### Example 45: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-3-fluoro-2-methylphenyl)ethyl)-1,2,4-oxadiazole-3-carboxamide

### Step 1: (R)—N-(1-(4-bromo-3-fluoro-2-methylphenyl)ethyl)-5-(tert-butyl)-1,2,4-oxadiazole-3-carboxamide

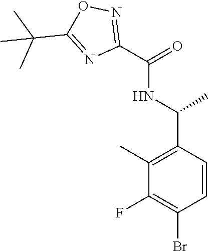 [SMILES: Cc1c([C@@H](C)NC(=O)c2noc(C(C)(C)C)n2)ccc(Br)c1F]

To a stirred solution of (1R)-1-(4-bromo-2,3-dimethylphenyl)ethanamine hydrochloride (2.50 g, 9.5 mmol) and sodium 5-tert-butyl-1,2,4-oxadiazole-3-carboxylate (2.18 g, 11.3 mmol) in DMF (30 mL) was added DIEA (4.94 mL, 38.2 mmol) dropwise at room temperature. The resulting mixture was stirred for 5 min at room temperature. To the above mixture was added T3P (17.98 g, 28.251 mmol) dropwise at room temperature. The resulting mixture was stirred for 2 h. The reaction was quenched with water (50 mL). The resulting mixture was extracted with EtOAc (2×100 mL). The combined organic layers were washed with brine (1×50 mL) and dried over anhydrous Na₂SO₄. After filtration, the filtrate was concentrated under reduced pressure. The residue was purified by silica gel column chromatography, eluted with PE/EtOAc (2:1) to afford the product (2 g, 55%). [M+H]⁺=386.1.

### Step 2: (R)-5-(tert-butyl)-N-(1-(3-fluoro-2-methyl-4-(4,4,5,5-tetramethyl-1,3,2-dioxaborolan-2-yl)phenyl)ethyl)-1,2,4-oxadiazole-3-carboxamide

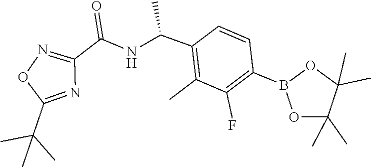 [SMILES: Cc1c([C@@H](C)NC(=O)c2noc(C(C)(C)C)n2)ccc(B2OC(C)(C)C(C)(C)O2)c1F]

To a stirred solution of (R)—N-(1-(4-bromo-3-fluoro-2-methylphenyl)ethyl)-5-(tert-butyl)-1,2,4-oxadiazole-3-carboxamide (800 mg, 2.08 mmol) and bis(pinacolato)diboron (793.05 mg, 3.12 mmol) in 1,4-dioxane (20 mL) were added KOAc (612.99 mg, 6.246 mmol) and Pd(dppf)Cl₂ (152.34 mg, 0.21 mmol) at room temperature. The resulting mixture was stirred for 16 h at 100° C. The mixture was allowed to cool down to room temperature. The reaction was quenched with water (20 mL). The resulting mixture was extracted with EtOAc (3×20 mL). The combined organic layers were washed with brine (1×20 mL) and dried over anhydrous Na₂SO₄. After filtration, the filtrate was concentrated under reduced pressure. The residue was purified by reverse phase flash chromatography with the following conditions: column, C18 silica gel; mobile phase, ACN in water (0.1% NH₄HCO₃), 50% to 80% gradient in 20 min; UV 254 nm to afford the product (75.4 mg, 9%). [M+H]⁺=432.2.

### Step 3: benzyl (R)-4-(4-(4-(1-(5-(tert-butyl)-1,2,4-oxadiazole-3-carboxamido)ethyl)-2-fluoro-3-methylphenyl)-6-methyl-9H-pyrimido[4,5-b]indol-7-yl)piperazine-1-carboxylate

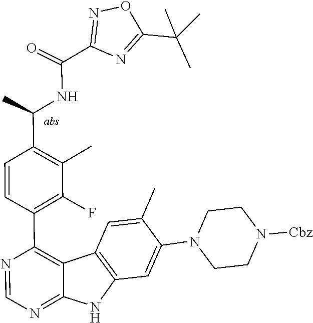 [SMILES: Cc1cc2c(cc1N1CCN(C(=O)OCc3ccccc3)CC1)[nH]c1ncnc(-c3ccc([C@@H](C)NC(=O)c4noc(C(C)(C)C)n4)c(C)c3F)c12]

A mixture of benzyl 4-(4-chloro-6-methyl-9H-pyrimido[4,5-b]indol-7-yl)piperazine-1-carboxylate (250 mg, 0.57 mmol), (R)-5-(tert-butyl)-N-(1-(3-fluoro-2-methyl-4-(4,4,5,5-tetramethyl-1,3,2-dioxaborolan-2-yl)phenyl)ethyl)-1,2,4-oxadiazole-3-carboxamide (296.1 mg, 0.68 mmol), Pd(dppf)Cl₂ (41.7 mg, 0.057 mmol), K₃PO₄ (243.7 mg, 1.15 mmol) in dioxane (10 mL) and H₂O (2 mL) was stirred at 80° C. overnight under a nitrogen atmosphere. The mixture was concentrated under vacuum. The residue was purified by silica gel column chromatography, eluted with EtOAc to afford the product (140 mg, 34.6%). [M+H]⁺=705.3.

### Step 4: (R)-5-(tert-butyl)-N-(1-(3-fluoro-2-methyl-4-(6-methyl-7-(piperazin-1-yl)-9H-pyrimido[4,5-b]indol-4-yl)phenyl)ethyl)-1,2,4-oxadiazole-3-carboxamide

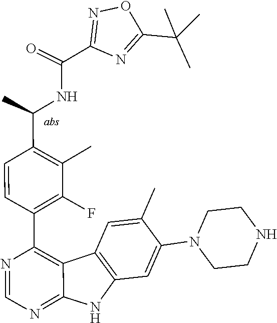 [SMILES: Cc1cc2c(cc1N1CCNCC1)[nH]c1ncnc(-c3ccc([C@@H](C)NC(=O)c4noc(C(C)(C)C)n4)c(C)c3F)c12]

A mixture of benzyl (R)-4-(4-(4-(1-(5-(tert-butyl)-1,2,4-oxadiazole-3-carboxamido)ethyl)-2-fluoro-3-methylphenyl)-6-methyl-9H-pyrimido[4,5-b]indol-7-yl)piperazine-1-carboxylate (140 mg, 0.19 mmol) in TFA (5 mL) was refluxed for 2 h. The mixture was concentrated under vacuum to afford the product (120 mg, crude), which was used in the next step directly. [M+H]⁺=571.3.

### Step 5: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methyl-9H-pyrimido[4,5-b]indol-4-yl)-3-fluoro-2-methylphenyl)ethyl)-1,2,4-oxadiazole-3-carboxamide

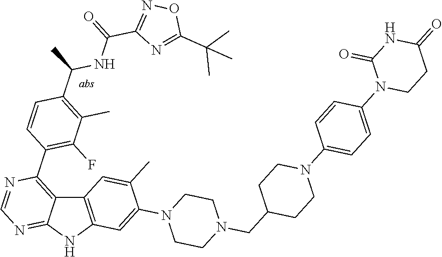 [SMILES: Cc1cc2c(cc1N1CCN(CC3CCN(c4ccc(N5CCC(=O)NC5=O)cc4)CC3)CC1)[nH]c1ncnc(-c3ccc([C@@H](C)NC(=O)c4noc(C(C)(C)C)n4)c(C)c3F)c12]

A mixture of (R)-5-(tert-butyl)-N-(1-(3-fluoro-2-methyl-4-(6-methyl-7-(piperazin-1-yl)-9H-pyrimido[4,5-b]indol-4-yl)phenyl)ethyl)-1,2,4-oxadiazole-3-carboxamide (214 mg, 0.37 mmol), 1-(4-(2,4-dioxotetrahydropyrimidin-1(2H)-yl)phenyl)piperidine-4-carbaldehyde (136 mg, 0.45 mmol) and AcOH (0.02 mL) in MeOH (10 mL) and DCM (10 mL) was stirred at rt overnight. Then, STAB (156.8 mg, 0.74 mmol) was added to the mixture above and stirred at rt for 5 h. The mixture was diluted with water (100 mL) and extracted with DCM (3×100 mL). The combined organic layers were dried over Na₂SO₄, filtered and concentrated under vacuum. The residue was purified by silica gel column chromatography, eluted with MeOH/DCM (1:7) to afford product (164.5 mg, 51.2%). ¹H NMR (400 MHz, DMSO) δ 12.29 (s, 1H), 10.27 (s, 1H), 9.61 (d, J=8.0 Hz, 1H), 8.90 (s, 1H), 7.60-7.50 (m, 2H), 7.19-7.09 (m, 4H), 6.93 (d, J=8.0 Hz, 2H), 5.52-5.41 (m, 1H), 3.75-3.64 (m, 4H), 3.01-2.87 (m, 4H), 2.74-2.61 (m, 5H), 2.60-2.51 (m, 2H), 2.41 (s, 3H), 2.30-2.18 (m, 5H), 1.92-1.88 (m, 2H), 1.87-1.78 (m, 2H), 1.76-1.66 (m, 1H), 1.58 (d, J=8.0 Hz, 2H), 1.43 (s, 9H), 1.30-1.17 (m, 2H); [M+H]⁺=856.4.

### Example 46: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-2-methylphenyl)ethyl)-1,2,4-oxadiazole-5-carboxamide

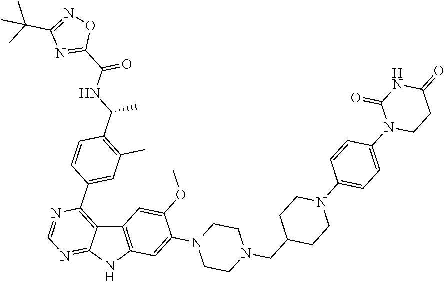 [SMILES: COc1cc2c(cc1N1CCN(CC3CCN(c4ccc(N5CCC(=O)NC5=O)cc4)CC3)CC1)[nH]c1ncnc(-c3ccc([C@@H](C)NC(=O)c4nc(C(C)(C)C)no4)c(C)c3)c12]

A mixture of (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide (130 mg, 0.23 mmol), 1-(4-(2,4-dioxotetrahydropyrimidin-1(2H)-yl)phenyl)piperidine-4-carbaldehyde (82.7 mg, 0.27 mmol) and AcOH (0.02 mL) in MeOH (5 mL) and DCM (5 mL) was stirred at rt overnight. Then, STAB (97 mg, 0.46 mmol) was added to the mixture above and stirred at rt for 5 h. The mixture was diluted with water (50 mL) and extracted with DCM (3×100 mL). The combined organic layers were dried over Na₂SO₄, filtered and concentrated under vacuum. The residue was purified by silica gel column chromatography, eluted with MeOH/DCM (1:7) to afford product (100.13 mg, 51.2%). ¹H NMR (400 MHz, DMSO) δ 12.16 (s, 1H), 10.26 (s, 1H), 9.94 (d, J=8.0 Hz, 1H), 8.85 (s, 1H), 7.83-7.68 (m, 3H), 7.28 (s, 1H), 7.13 (d, J=12.0 Hz, 2H), 7.02 (s, 1H), 6.93 (d, J=8.0 Hz, 2H), 5.45-5.35 (m, 1H), 3.76-3.62 (m, 7H), 3.15-3.03 (m, 4H), 2.74-2.61 (m, 5H), 2.60-2.54 (m, 6H), 2.30-2.18 (m, 2H), 1.88-1.78 (m, 2H), 1.77-1.66 (m, 1H), 1.57 (d, J=8.0 Hz, 3H), 1.37 (s, 9H), 1.31-1.16 (m, 2H); [M+H]⁺=854.4.

### Example 47: 3-(tert-butyl)-N-((1R)-1-(4-(7-(4-((3-(4-(2,6-dioxopiperidin-3-yl)phenyl)cyclobutyl)methyl)piperazin-1-yl)-6-methoxy-9H-pyrimido[4,5-b]indol-4-yl)-2-methylphenyl)ethyl)-1,2,4-oxadiazole-5-carboxamide

### Step 1: benzyl 4-(4-chloro-2-methoxyphenyl)piperazine-1-carboxylate

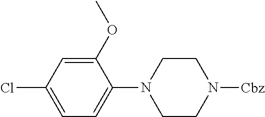 [SMILES: COc1cc(Cl)ccc1N1CCN(C(=O)OCc2ccccc2)CC1]

To a solution of 1-bromo-4-chloro-2-methoxybenzene (50.00 g, 225.7 mmol) and benzyl piperazine-1-carboxylate (59.67 g, 270.9 mmol) in toluene (1.0 L) were added BINAP (28.11 g, 45.2 mmol), Pd₂(dba)₃ (20.67 g, 22.6 mmol) and t-BuONa (43.39 g, 451.5 mmol). After stirring for 2 h at 95° C. under a nitrogen atmosphere, the resulting mixture was filtered. The filter cake was washed with EA (3×200 mL). The filtrate was concentrated under reduced pressure. The residue was purified by silica gel column chromatography, eluted with PE/EtOAc (20:1 to 3:1) to afford the product (50 g, 61.4%). [M+H]⁺=361.1.

### Step 2: 1-(4-chloro-2-methoxy-5-nitrophenyl)piperazine

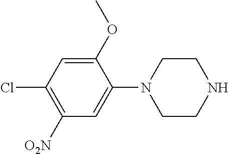 [SMILES: COc1cc(Cl)c([N+](=O)[O-])cc1N1CCNCC1]

To a stirred solution of benzyl 4-(4-chloro-2-methoxyphenyl)piperazine-1-carboxylate (40.0 g, 110.8 mmol) in conc. H₂SO₄ (200.0 mL, 3.75 mol) was added guanidine nitrate (16.24 g, 133.0 mmol) in portions at 0° C. under nitrogen atmosphere. The resulting mixture was stirred for 2 h at 0° C. under nitrogen atmosphere. The reaction was quenched by addition of sat. Na₂CO₃ (aq.) at 0° C. The resulting mixture was extracted with EtOAc (6×500 mL) and DCM (10% CH₃OH in DCM, 3×500 mL). The combined organic layers were washed with brine (100 mL) and dried over anhydrous Na₂SO₄. After filtration, the filtrate was concentrated under reduced pressure to give the product (30 g, crude). [M+H]⁺=272.1.

### Step 3: benzyl 4-(4-chloro-2-methoxy-5-nitrophenyl)piperazine-1-carboxylate

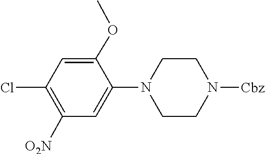 [SMILES: COc1cc(Cl)c([N+](=O)[O-])cc1N1CCN(C(=O)OCc2ccccc2)CC1]

To a stirred solution of 1-(4-chloro-2-methoxy-5-nitrophenyl)piperazine (37.0 g, 136.2 mmol) and DIEA (71.16 mL, 408.5 mmol) in DCM (400.0 mL) was added Cbz-Cl (46.46 g, 272.4 mmol) dropwise at 0° C. under nitrogen atmosphere. The resulting mixture was stirred for 16 h at room temperature and concentrated under reduced pressure. The residue was purified by silica gel column chromatography, eluted with PE/EtOAc (15:1 to 3:1) to afford the product (26.5 g, 47.9%). [M+H]⁺=406.1.

### Step 4: benzyl 4-(4-(1-cyano-2-ethoxy-2-oxoethyl)-2-methoxy-5-nitrophenyl)piperazine-1-carboxylate

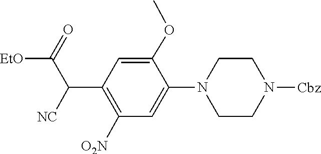 [SMILES: CCOC(=O)C(C#N)c1cc(OC)c(N2CCN(C(=O)OCc3ccccc3)CC2)cc1[N+](=O)[O-]]

To a stirred mixture of Cs₂CO₃ (63.82 g, 195.9 mmol) in DMF (500.0 mL) was added ethyl cyanoacetate (8.86 g, 78.3 mmol) dropwise at 0° C. under nitrogen atmosphere. The resulting mixture was stirred for 0.5 h at 0° C. To the above mixture was added a solution of benzyl 4-(4-chloro-2-methoxy-5-nitrophenyl)piperazine-1-carboxylate (26.50 g, 65.3 mmol) in DMF (30.0 mL) dropwise at 0° C. The resulting mixture was stirred for additional 16 h at 80° C. The mixture was filtered, and the filter cake was washed with EtOAc (3×500 mL). The solution was acidified to pH 5-6 with HCl (aq., 1.0 M). The resulting solution was extracted with EtOAc (3×500.0 mL). The combined organic layers were washed with brine (500.0 mL) and dried over anhydrous Na₂SO₄. After filtration, the filtrate was concentrated under reduced pressure. The residue was purified by silica gel column chromatography, eluted with PE/EA (10:1 to 1:1) to afford the product (28.2 g, 89.5%). [M+H]⁺=483.1.

### Step 5: ethyl 2-amino-6-(4-((benzyloxy)carbonyl)piperazin-1-yl)-5-methoxy-1H-indole-3-carboxylate

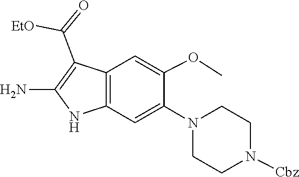 [SMILES: CCOC(=O)c1c(N)[nH]c2cc(N3CCN(C(=O)OCc4ccccc4)CC3)c(OC)cc12]

To a stirred solution of benzyl 4-(4-(1-cyano-2-ethoxy-2-oxoethyl)-2-methoxy-5-nitrophenyl)piperazine-1-carboxylate (28.2 g, 58.4 mmol) in AcOH (282.0 mL) was added Fe (22.85 g, 0.41 mmol) at room temperature. The resulting mixture was stirred for 16 h at room temperature. The reaction was monitored by LCMS and concentrated under reduced pressure. The residue was purified by silica gel column chromatography, eluted with CH₂Cl₂/MeOH (20:1) to afford the product (24.3 g, 91.9%). [M+H]⁺=453.1.

### Step 6: benzyl 4-(6-methoxy-4-oxo-4,9-dihydro-3H-pyrimido[4,5-b]indol-7-yl)piperazine-1-carboxylate

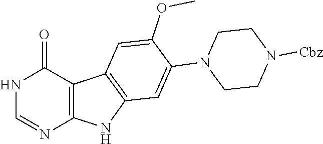 [SMILES: COc1cc2c(cc1N1CCN(C(=O)OCc3ccccc3)CC1)[nH]c1nc[nH]c(=O)c12]

To a stirred solution of ethyl 2-amino-6-(4-((benzyloxy)carbonyl)piperazin-1-yl)-5-methoxy-1H-indole-3-carboxylate (33.1 g, 73.1 mmol) in 2-methoxyethan-1-ol (330.0 mL) was added acetic acid methanimidamide (76.1 g, 731.0 mmol) in portions at room temperature under air atmosphere. The resulting mixture was stirred for 16 h at 125° C. under nitrogen atmosphere. The mixture was cooled to room temperature and diluted with water (800.0 mL). The precipitated solids were collected by filtration and washed with water (2×50.0 mL). The resulting solid was dried under infrared light to give the product (28 g, 88.3%). [M+H]⁺=434.1.

### Step 7: benzyl 4-(4-chloro-6-methoxy-9H-pyrimido[4,5-b]indol-7-yl)piperazine-1-carboxylate

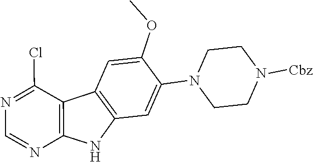 [SMILES: COc1cc2c(cc1N1CCN(C(=O)OCc3ccccc3)CC1)[nH]c1ncnc(Cl)c12]

To a stirred solution of benzyl 4-(6-methoxy-4-oxo-4,9-dihydro-3H-pyrimido[4,5-b]indol-7-yl)piperazine-1-carboxylate (28.0 g, 64.6 mmol) in dioxane (560.0 mL) was added POCl₃ (78.27 mL, 0.84 mol) at room temperature under air atmosphere. The resulting mixture was stirred for 6 h at 90° C. under nitrogen atmosphere. The mixture was cooled to room temperature and concentrated under reduced pressure. The residue was basified to pH 7 with saturated NaHCO₃ (aq.). The resulting mixture was extracted with EtOAc (2×1.0 L) and dried over anhydrous Na₂SO₄. After filtration, the filtrate was concentrated under reduced pressure. The precipitated solids were collected by filtration and washed with diethyl ether (3×100.0 mL) to give the product (30 g, 96.0%). ¹H NMR (400 MHz, DMSO) δ 12.52 (s, 1H), 8.66 (s, 1H), 7.68 (s, 1H), 7.42-7.29 (m, 5H), 7.07 (s, 1H), 5.13 (s, 2H), 3.93 (s, 3H), 3.61 (s, 4H), 3.09 (t, J=5.1 Hz, 4H); [M+H]⁺=452.3.

### Step 8: benzyl (R)-4-(4-(4-(1-(3-(tert-butyl)-1,2,4-oxadiazole-5-carboxamido)ethyl)-3-methylphenyl)-6-methoxy-9H-pyrimido[4,5-b]indol-7-yl)piperazine-1-carboxylate

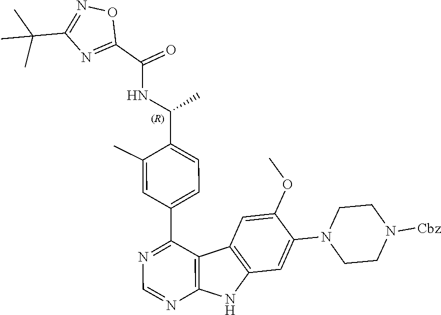

To a stirred solution of benzyl 4-(4-chloro-6-methoxy-9H-pyrimido[4,5-b]indol-7-yl)piperazine-1-carboxylate (1.1 g, 2.4 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (1.21 g, 2.9 mmol) in DMF/H₂O (5/1, 20 mL) were added K₃PO₄ (1.04 g, 4.9 mmol) and Pd(dppf)Cl₂ (0.18 g, 0.24 mmol) at room temperature under nitrogen atmosphere. The resulting mixture was stirred overnight at 90° C. under nitrogen atmosphere. The mixture was cooled to room temperature and diluted with water (250.0 mL). The resulting mixture was extracted with EtOAc (3×300.0 mL). The combined organic layers were washed with water (1×300.0 mL) and dried over anhydrous Na₂SO₄. After filtration, the filtrate was concentrated under reduced pressure. The residue was purified by silica gel column chromatography, eluted with CH₂Cl₂/MeOH (20:1) to afford the product (850 mg, 49.6%). [M+H]⁺=703.4.

### Step 9: (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide formate

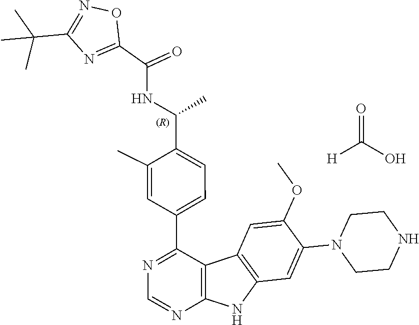

A solution of benzyl (R)-4-(4-(4-(1-(3-(tert-butyl)-1,2,4-oxadiazole-5-carboxamido)ethyl)-3-methylphenyl)-6-methoxy-9H-pyrimido[4,5-b]indol-7-yl)piperazine-1-carboxylate (0.7 g, 0.996 mmol) in TFA (65.0 mL) was stirred overnight at 60° C. under nitrogen atmosphere. The resulting mixture was concentrated under reduced pressure. The residue was purified by reverse phase flash chromatography with the following conditions: column, silica gel; mobile phase, 0.10% formic acid in water, 10% to 50% gradient in 30 min; Detector, UV 254 nm. This resulted the desired product (530 mg, 86.6%). ¹H NMR (400 MHz, DMSO) δ 12.25 (s, 1H), 9.97 (d, J=7.6 Hz, 1H), 8.88 (s, 1H), 8.24 (s, 1H), 7.83-7.75 (m, 2H), 7.73 (d, J=7.9 Hz, 1H), 7.30 (s, 1H), 7.05 (s, 1H), 5.44-5.34 (m, 1H) 3.67 (s, 3H), 3.19 (s, 8H), 2.53 (s, 3H), 1.58 (d, J=7.0 Hz, 3H), 1.37 (s, 9H); [M+H]⁺=569.1.

### Step 10: 3-(tert-butyl)-N-((1R)-1-(4-(7-(4-((3-(4-(2,6-dioxopiperidin-3-yl)phenyl)cyclobutyl)methyl)piperazin-1-yl)-6-methoxy-9H-pyrimido[4,5-b]indol-4-yl)-2-methylphenyl)ethyl)-1,2,4-oxadiazole-5-carboxamide

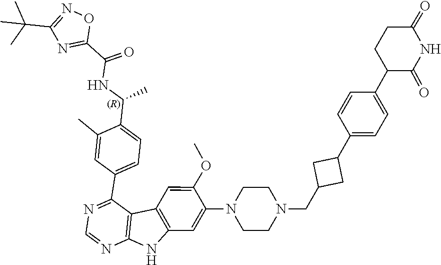

A stirred solution of (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide formate (0.1 g, 0.163 mmol), 3-(4-(2,6-dioxopiperidin-3-yl)phenyl)cyclobutane-1-carbaldehyde (50 mg, 0.184 mmol) and AcOH (0.5 mL) in DCM/MeOH(10.0 m L/2.5 mL) was stirred for 16 h at room temperature, then NaBH(OAc)₃ (173 mg, 0.815 mmol) was added and stirred at room temperature for 1 h. The resulting mixture was diluted with water (20.0 mL) and extracted with DCM (3×15.0 mL). The combined organic layers were concentrated under reduced pressure. The residue was purified by silica gel column chromatography, eluted with CH₂Cl₂/MeOH (10:1) to afford the product (66 mg, 49.2%). ¹H NMR (500 MHz, DMSO) δ 12.15 (s, 1H), 10.82 (s, 1H), 9.95 (d, J=7.7 Hz, 1H), 8.85 (s, 1H), 7.82-7.75 (m, 2H), 7.72 (d, J=8.0 Hz, 1H), 7.20 (ddd, J=24.4, 17.9, 9.9 Hz, 5H), 7.01 (s, 1H), 5.39 (q, J=6.9 Hz, 1H), 3.87-3.76 (m, 1H), 3.65 (s, 3H), 3.42-3.33 (m, 1H), 3.07 (s, 4H), 2.73-2.51 (m, 9H), 2.50-2.40 (m, 4H), 2.25-2.11 (m, 2H), 2.02 (dt, J=13.2, 4.6 Hz, 1H), 1.81-1.69 (m, 1H), 1.57 (d, J=6.9 Hz, 3H), 1.37 (s, 9H); [M+H]⁺=824.9.

### Example 48: 3-(tert-butyl)-N-((1R)-1-(4-(7-(4-(4-(4-(2,6-dioxopiperidin-3-yl)phenyl)cyclohexyl)piperazin-1-yl)-6-methoxy-9H-pyrimido[4,5-b]indol-4-yl)-2-methylphenyl)ethyl)-1,2,4-oxadiazole-5-carboxamide

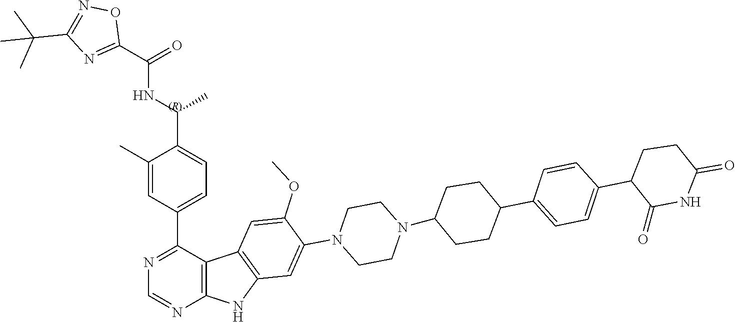

The titled compound was synthesized in the procedures similar to Example 47. ¹H NMR (500 MHz, DMSO) δ 12.31 (s, 1H), 10.84 (s, 1H), 10.01 (d, J=7.6 Hz, 1H), 8.93 (s, 1H), 7.88-7.80 (m, 2H), 7.78 (d, J=7.9 Hz, 1H), 7.35 (s, 1H), 7.12 (d, J=7.7 Hz, 3H), 7.00 (d, J=6.0 Hz, 2H), 5.45 (q, J=7.0 Hz, 1H), 4.02-3.84 (m, 2H), 3.84-3.76 (m, 2H), 3.77-3.66 (m, 5H), 3.46 (d, J=26.8 Hz, 2H), 3.36-3.26 (m, 2H), 3.25-3.09 (m, 3H), 2.84-2.65 (m, 4H), 2.59 (d, J=5.0 Hz, 3H), 2.23 (ddd, J=23.8, 15.8, 4.9 Hz, 3H), 2.11-2.02 (m, 1H), 1.92-1.78 (m, 1H), 1.63 (d, J=6.9 Hz, 3H), 1.43 (s, 9H); [M+H]⁺=839.6.

### Example 49: 3-(tert-butyl)-N—((R)-1-(4-(7-(4-(3-(4-((S)-2,6-dioxopiperidin-3-yl)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide

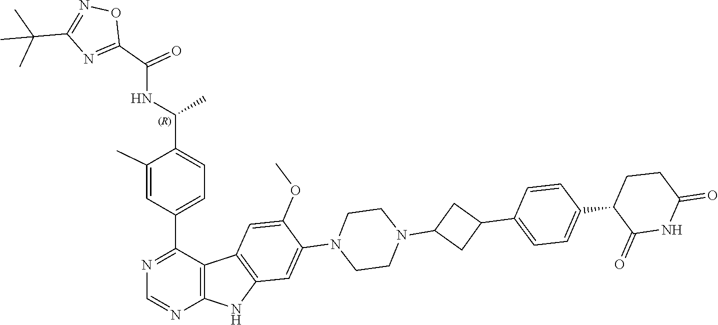

The titled compound was synthesized in the procedures similar to Example 47. ¹H NMR (500 MHz, DMSO) δ 12.15 (s, 1H), 10.83 (s, 1H), 9.95 (d, J=7.7 Hz, 1H), 8.85 (s, 1H), 7.84-7.75 (m, 2H), 7.72 (d, J=7.9 Hz, 1H), 7.28 (s, 1H), 7.25-7.12 (m, 4H), 7.02 (s, 1H), 5.39 (q, J=7.0 Hz, 1H), 3.82 (dd, J=11.3, 4.8 Hz, 1H), 3.66 (s, 3H), 3.20-2.99 (m, 5H), 2.84-2.73 (m, 1H), 2.67 (ddd, J=17.0, 13.4, 7.6 Hz, 1H), 2.53 (s, 4H), 2.46 (t, J=11.7 Hz, 6H), 2.18 (dt, J=20.2, 9.9 Hz, 1H), 2.07-1.98 (m, 1H), 1.95-1.82 (m, 2H), 1.58 (d, J=6.9 Hz, 3H), 1.37 (s, 9H); [M+H]⁺=810.6.

### Example 50: 3-(tert-butyl)-N—((R)-1-(4-(7-(4-(3-(4-((R)-2,6-dioxopiperidin-3-yl)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide

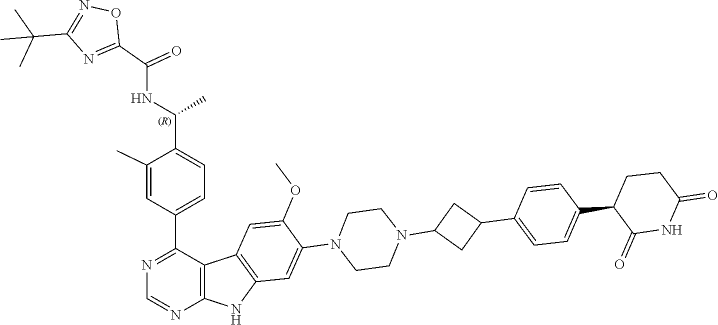

The titled compound was synthesized in the procedures similar to Example 47. ¹H NMR (500 MHz, DMSO) δ 12.16 (s, 1H), 10.83 (s, 1H), 9.96 (d, J=7.7 Hz, 1H), 8.85 (s, 1H), 7.83-7.76 (m, 2H), 7.72 (d, J=8.0 Hz, 1H), 7.28 (s, 1H), 7.19 (dd, J=28.3, 7.4 Hz, 4H), 7.02 (s, 1H), 5.43-5.36 (m, 1H), 3.82 (dd, J=11.5, 4.9 Hz, 1H), 3.66 (s, 3H), 3.20-3.04 (m, 4H), 2.78 (dt, J=15.0, 7.6 Hz, 1H), 2.71-2.61 (m, 1H), 2.53 (s, 4H), 2.47 (d, J=4.3 Hz, 6H), 2.22-2.11 (m, 1H), 2.06-1.99 (m, 1H), 1.95-1.83 (m, 2H), 1.57 (d, J=6.9 Hz, 3H), 1.37 (s, 9H); [M+H]⁺=810.6.

### Example 51: 3-(tert-butyl)-N-((1R)-1-(4-(7-(4-(1-(3-(2,6-dioxopiperidin-3-yl)phenyl)pyrrolidin-3-yl)piperazin-1-yl)-6-methoxy-9H-pyrimido[4,5-b]indol-4-yl)-2-methylphenyl)ethyl)-1,2,4-oxadiazole-5-carboxamide

### Step 1: 1-(3-(2,6-bis(benzyloxy)pyridin-3-yl)phenyl)pyrrolidin-3-ol

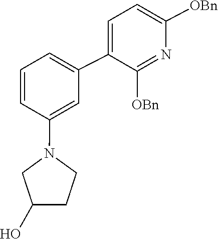 [SMILES: OC1CCN(c2cccc(-c3ccc(OCc4ccccc4)nc3OCc3ccccc3)c2)C1]

To a stirred mixture of 2,6-bis(benzyloxy)-3-(3-bromophenyl)pyridine (4.1 g, 9.21 mmol) and pyrrolidin-3-ol hydrochloride (1.36 g, 11.06 mmol) in DME (80.0 mL) were added BINAP (573 mg, 0.921 mmol), Pd₂(dba)₃ (422 mg, 0.46 mmol) and t-BuONa (2.65 g, 27.63 mmol). After stirring for 6 h at 100° C. under a nitrogen atmosphere, the resulting mixture was concentrated under reduced pressure. The residue was purified by silica gel column chromatography, eluted with PE/EtOAc (10:1 to 3:1) to afford the product (3.2 g, 76.9%). [M+H]⁺=453.3.

### Step 2: 3-(3-(3-hydroxypyrrolidin-1-yl)phenyl)piperidine-2,6-dione

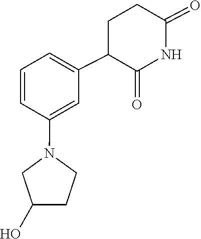 [SMILES: O=C1CCC(c2cccc(N3CCC(O)C3)c2)C(=O)N1]

A solution of 1-(3-(2,6-bis(benzyloxy)pyridin-3-yl)phenyl)pyrrolidin-3-ol (2.9 g, 33.606 mmol) and Pd/C (2.9 g) in methanol (80.0 mL) was stirred for 16 h at room temperature under hydrogen atmosphere. The mixture was filtered and concentrated under reduced pressure to afford the product (1.7 g, 97%). [M+H]⁺=275.2.

### Step 3: 3-(3-(3-oxopyrrolidin-1-yl)phenyl)piperidine-2,6-dione

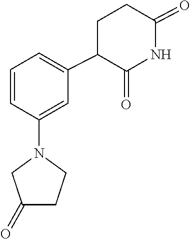 [SMILES: O=C1CCN(c2cccc(C3CCC(=O)NC3=O)c2)C1]

To a stirred mixture of DMSO (1.48 g, 19 mmol) in DCM (10.0 mL) was added oxalyl chloride (9.5 mL, 9.5 mmol) at −78° C. After stirring for 1 h at −78° C. under a nitrogen atmosphere, 3-(3-(3-hydroxypyrrolidin-1-yl)phenyl)piperidine-2,6-dione (1.3 g, 4.74 mmol) in DCM (10.0 mL) was added at −78° C. The resulting mixture was stirred for 1 h at −78° C., then TEA (2.4 g, 23.7 mmol) was added and stirred for 30 min at room temperature. The resulting mixture was extracted with DCM (3×50.0 mL) and H₂O (3×50.0 mL). The combined organic layers were washed with brine (50 mL) and dried over anhydrous Na₂SO₄. After filtration, the filtrate was concentrated under reduced pressure. The residue was purified by silica gel column chromatography, eluted with DCM/MeOH (20:1) to afford the product (1.1 g, 84.6%). [M+H]⁺=273.

### Step 4: 3-(tert-butyl)-N-((1R)-1-(4-(7-(4-(1-(3-(2,6-dioxopiperidin-3-yl)phenyl)pyrrolidin-3-yl)piperazin-1-yl)-6-methoxy-9H-pyrimido[4,5-b]indol-4-yl)-2-methylphenyl)ethyl)-1,2,4-oxadiazole-5-carboxamide

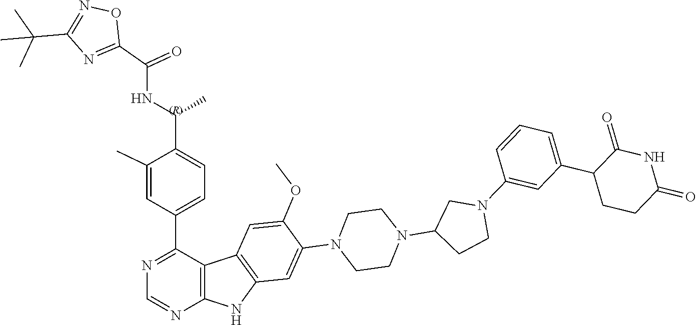

A solution of (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide formate (120 mg, 0.195 mmol), 3-(3-(3-oxopyrrolidin-1-yl)phenyl)piperidine-2,6-dione (106 mg, 0.39 mmol) and HOAc (1.0 mL) in MeOH (3.0 mL) and DCM (12.0 mL) was stirred for 72 h at 35° C., then NaBH(OAc)₃ (413.4 mg, 1.95 mmol) was added and stirred at room temperature for 24 h. The resulting mixture was diluted with water (20.0 mL) and extracted with DCM (3×15.0 mL). The combined organic layers were concentrated under reduced pressure. The residue was purified by silica gel column chromatography, eluted with CH₂Cl₂/MeOH (20:1) and prep-HPLC to afford the product (10 mg, 6.2%). ¹H NMR (500 MHz, MeOD) δ 8.80 (s, 1H), 7.76-7.69 (m, 3H), 7.24 (s, 1H), 7.21-7.14 (m, 2H), 6.57-6.49 (m, 2H), 6.47 (s, 1H), 5.53 (q, J=7.0 Hz, 1H), 3.80 (dd, J=9.3, 5.6 Hz, 1H), 3.68 (s, 3H), 3.58 (t, J=7.5 Hz, 1H), 3.47 (dd, J=18.0, 9.4 Hz, 1H), 3.24-3.12 (m, 6H), 2.95-2.77 (m, 4H), 2.73-2.55 (m, 6H), 2.38-2.32 (m, 1H), 2.30-2.18 (m, 2H), 2.06-1.95 (m, 1H), 1.66 (d, J=7.0 Hz, 3H), 1.42 (s, 9H); [M+H]⁺=825.6.

### Example 52: (R)—N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-2-methylphenyl)ethyl)-3-(1-methylcyclopropyl)-1,2,4-oxadiazole-5-carboxamide

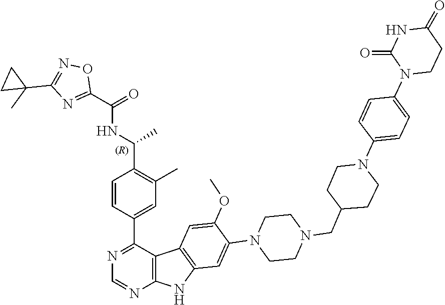

The titled compound was synthesized in the procedures similar to Example 47. ¹H NMR (500 MHz, DMSO) δ 12.10 (s, 1H), 10.19 (s, 1H), 9.80 (d, J=7.7 Hz, 1H), 8.78 (s, 1H), 7.74-7.68 (m, 2H), 7.63 (d, J=7.9 Hz, 1H), 7.20 (s, 1H), 7.07 (d, J=8.9 Hz, 2H), 6.95 (s, 1H), 6.86 (d, J=9.0 Hz, 2H), 5.30 (q, J=7.0 Hz, 1H), 3.62 (dd, J=11.9, 5.2 Hz, 4H), 3.58 (s, 3H), 3.09-2.93 (m, 4H), 2.60 (dd, J=16.5, 9.6 Hz, 4H), 2.51-2.46 (m, 4H), 2.45 (s, 3H), 2.17 (d, J=7.0 Hz, 2H), 1.83 (s, 2H), 1.75 (d, J=11.4 Hz, 2H), 1.70-1.60 (m, 1H), 1.49 (d, J=6.9 Hz, 3H), 1.42 (s, 3H), 1.19-1.11 (m, 6H), 0.92 (d, J=2.5 Hz, 2H); [M+H]⁺=852.6.

### Example 53: (R)—N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-3-fluoro-2-methylphenyl)ethyl)-3-(1-methylcyclopropyl)-1,2,4-oxadiazole-5-carboxamide

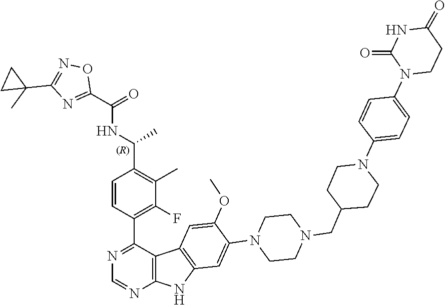

The titled compound was synthesized in the procedures similar to Example 47. ¹H NMR (500 MHz, DMSO) δ 12.18 (s, 1H), 10.25 (s, 1H), 9.93 (d, J=6.9 Hz, 1H), 8.88 (s, 1H), 7.62-7.45 (m, 2H), 7.13 (d, J=7.4 Hz, 2H), 7.01 (s, 1H), 6.93 (d, J=7.7 Hz, 2H), 6.72 (s, 1H), 5.43-5.31 (m, 1H), 3.74-3.64 (m, 4H), 3.56 (s, 3H), 3.13-3.02 (m, 4H), 2.71-2.63 (m, 4H), 2.41 (s, 4H), 2.29-2.19 (m, 2H), 1.87-1.77 (m, 2H), 1.75-1.66 (m, 1H), 1.57 (d, J=5.5 Hz, 3H), 1.49 (s, 3H), 1.30-1.16 (m, 4H), 1.11 (s, 3H), 1.04-0.94 (m, 2H); [M+H]⁺=870.5.

### Example 54: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-3-fluoro-2-methylphenyl)ethyl)-1,2,4-oxadiazole-5-carboxamide

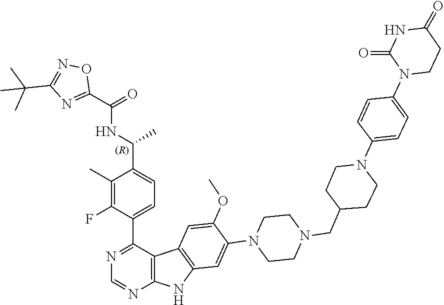

The titled compound was synthesized in the procedures similar to Example 47. ¹H NMR (500 MHz, DMSO) δ 12.19 (s, 1H), 10.26 (s, 1H), 10.01 (d, J=7.5 Hz, 1H), 8.89 (s, 1H), 7.56 (q, J=8.1 Hz, 2H), 7.13 (d, J=8.9 Hz, 2H), 7.01 (s, 1H), 6.93 (d, J=9.0 Hz, 2H), 6.72 (s, 1H), 5.40 (q, J=7.0 Hz, 1H), 3.69 (t, J=6.7 Hz, 4H), 3.56 (s, 3H), 3.19-2.95 (m, 4H), 2.67 (dd, J=17.5, 10.5 Hz, 4H), 2.53 (dd, J=31.1, 14.9 Hz, 4H), 2.42 (d, J=1.4 Hz, 3H), 2.23 (d, J=6.7 Hz, 2H), 1.82 (d, J=11.9 Hz, 2H), 1.76-1.65 (m, 1H), 1.58 (d, J=6.9 Hz, 3H), 1.37 (s, 9H), 1.23 (dd, J=21.3, 11.3 Hz, 2H); [M+H]⁺=872.6.

### Example 55: 3-(tert-butyl)-N—((R)-1-(4-(7-(4-(3-(4-((S)-2,6-dioxopiperidin-3-yl)phenyl)cyclobutyl)piperazin-1-yl)-6-methoxy-9H-pyrimido[4,5-b]indol-4-yl)-3-fluoro-2-methylphenyl)ethyl)-1,2,4-oxadiazole-5-carboxamide

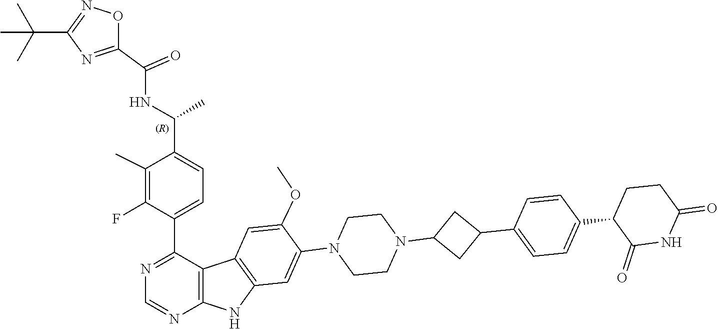

The titled compound was synthesized in the procedures similar to Example 47. ¹H NMR (500 MHz, DMSO) δ 12.17 (s, 1H), 10.82 (s, 1H), 10.00 (d, J=7.1 Hz, 1H), 8.89 (s, 1H), 7.61-7.50 (m, 2H), 7.24-7.13 (m, 4H), 7.02 (s, 1H), 6.73 (s, 1H), 5.49-5.32 (m, 1H), 3.82 (dd, J=11.7, 4.8 Hz, 1H), 3.56 (s, 3H), 3.10 (ddd, J=19.2, 16.1, 10.6 Hz, 4H), 2.78 (dt, J=16.0, 11.4 Hz, 1H), 2.71-2.62 (m, 1H), 2.61-2.51 (m, 3H), 2.50-2.34 (m, 8H), 2.23-2.13 (m, 1H), 2.07-1.99 (m, 1H), 1.93-1.84 (m, 2H), 1.59 (d, J=6.5 Hz, 3H), 1.38 (d, J=1.2 Hz, 9H); [M+H]⁺=828.5.

### Example 56: 3-(tert-butyl)-N—((R)-1-(4-(7-(4-(3-(4-((R)-2,6-dioxopiperidin-3-yl)phenyl)cyclobutyl)piperazin-1-yl)-6-methoxy-9H-pyrimido[4,5-b]indol-4-yl)-3-fluoro-2-methylphenyl)ethyl)-1,2,4-oxadiazole-5-carboxamide

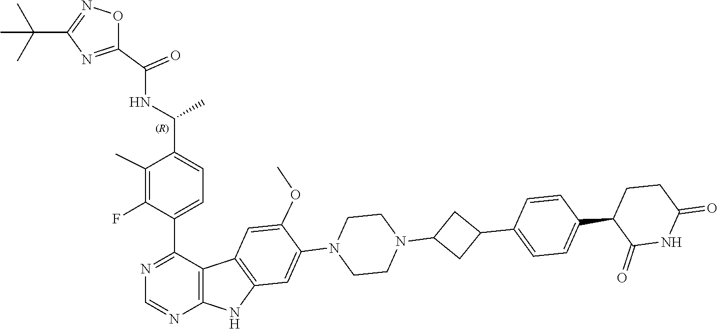

The titled compound was synthesized in the procedures similar to Example 47. ¹H NMR (500 MHz, DMSO) δ 12.17 (s, 1H), 10.82 (s, 1H), 10.00 (d, J=7.4 Hz, 1H), 8.89 (s, 1H), 7.56 (q, J=8.0 Hz, 2H), 7.23-7.13 (m, 4H), 7.01 (s, 1H), 6.73 (s, 1H), 5.44-5.36 (m, 1H), 3.82 (dd, J=11.4, 4.8 Hz, 1H), 3.56 (s, 3H), 3.16-3.11 (m, 4H), 2.85-2.73 (m, 1H), 2.65-2.61 (m, 1H), 2.60-2.52 (m, 3H), 2.50-2.40 (m, 8H), 2.23-2.13 (m, 1H), 2.06-1.99 (m, 1H), 1.92-1.83 (m, 2H), 1.58 (d, J=6.9 Hz, 3H), 1.37 (s, 9H); [M+H]⁺=828.5.

### Example 57: 3-(tert-butyl)-N-((1R)-1-(4-(7-(4-(1-(3-(2,6-dioxopiperidin-3-yl)phenyl)pyrrolidin-3-yl)piperazin-1-yl)-6-methoxy-9H-pyrimido[4,5-b]indol-4-yl)-3-fluoro-2-methylphenyl)ethyl)-1,2,4-oxadiazole-5-carboxamide

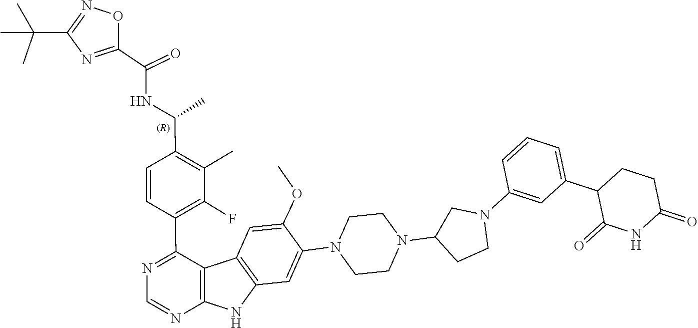

The titled compound was synthesized in the procedures similar to Example 47. ¹H NMR (500 MHz, DMSO) δ 12.17 (s, 1H), 10.78 (s, 1H), 10.00 (d, J=7.5 Hz, 1H), 8.89 (s, 1H), 7.56 (q, J=8.1 Hz, 2H), 7.11 (t, J=7.8 Hz, 1H), 7.02 (s, 1H), 6.73 (s, 1H), 6.45 (t, J=6.9 Hz, 2H), 6.41 (s, 1H), 5.40 (q, J=6.8 Hz, 1H), 3.73 (dd, J=10.9, 4.9 Hz, 1H), 3.57 (s, 3H), 3.49 (dd, J=15.7, 7.3 Hz, 1H), 3.36 (dd, J=14.0, 7.8 Hz, 1H), 3.29 (s, 1H), 3.23 (ddd, J=12.9, 9.7, 3.9 Hz, 1H), 3.16-3.03 (m, 4H), 3.00 (dd, J=15.1, 7.3 Hz, 1H), 2.73-2.59 (m, 5H), 2.47-2.39 (m, 4H), 2.27-2.15 (m, 2H), 2.09-2.00 (m, 1H), 1.91-1.82 (m, 1H), 1.58 (d, J=7.0 Hz, 3H), 1.38 (s, 9H); [M+H]⁺=843.5.

### Example 58: (R)-5-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-3-fluoro-2-methylphenyl)ethyl)-1,2,4-oxadiazole-3-carboxamide

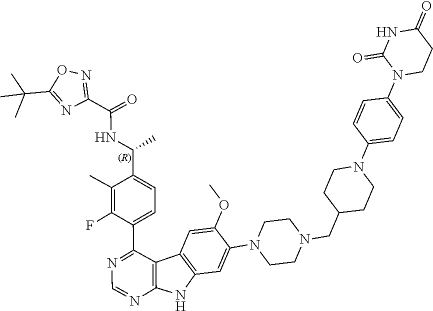

The titled compound was synthesized in the procedures similar to Example 47. ¹H NMR (500 MHz, DMSO) δ 12.19 (s, 1H), 10.27 (s, 1H), 9.62 (d, J=7.3 Hz, 1H), 8.89 (s, 1H), 7.58-7.49 (m, 2H), 7.13 (d, J=8.4 Hz, 2H), 7.01 (s, 1H), 6.93 (d, J=8.5 Hz, 2H), 6.73 (s, 1H), 5.50-5.33 (m, 1H), 3.73-3.65 (m, 4H), 3.57 (s, 3H), 3.15-2.98 (m, 4H), 2.67 (dd, J=17.0, 9.8 Hz, 4H), 2.64-2.57 (m, 4H), 2.42 (s, 3H), 2.23 (d, J=5.9 Hz, 2H), 1.82 (d, J=10.6 Hz, 2H), 1.76-1.66 (m, 1H), 1.56 (d, J=6.7 Hz, 3H), 1.43 (s, 2H), 1.23 (dd, J=22.2, 11.2 Hz, 2H); [M+H]⁺=872.6.

### Example 59: 5-(tert-butyl)-N—((R)-1-(4-(7-(4-(3-(4-((S)-2,6-dioxopiperidin-3-yl)phenyl)cyclobutyl)piperazin-1-yl)-6-methoxy-9H-pyrimido[4,5-b]indol-4-yl)-3-fluoro-2-methylphenyl)ethyl)-1,2,4-oxadiazole-3-carboxamide

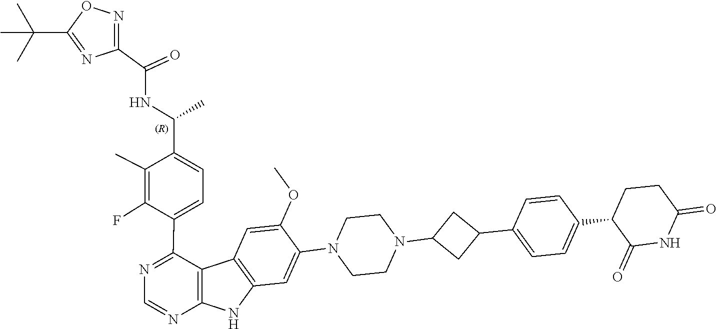

The titled compound was synthesized in the procedures similar to Example 47. ¹H NMR (500 MHz, DMSO) δ 12.17 (s, 1H), 10.82 (s, 1H), 9.60 (d, J=7.6 Hz, 1H), 8.89 (s, 1H), 7.58-7.51 (m, 2H), 7.24-7.13 (m, 4H), 7.02 (s, 1H), 6.73 (s, 1H), 5.45-5.37 (m, 1H), 3.82 (dd, J=11.2, 4.6 Hz, 1H), 3.57 (s, 3H), 3.19-3.04 (m, 4H), 2.83-2.74 (m, 1H), 2.69-2.62 (m, 1H), 2.60-2.50 (m, 3H), 2.48-2.35 (m, 8H), 2.23-2.13 (m, 1H), 2.06-1.99 (m, 1H), 1.91-1.81 (m, 2H), 1.56 (d, J=6.9 Hz, 3H), 1.43 (s, 9H); [M+H]⁺=828.6.

### Example 60: 5-(tert-butyl)-N—((R)-1-(4-(7-(4-(3-(4-((R)-2,6-dioxopiperidin-3-yl)phenyl)cyclobutyl)piperazin-1-yl)-6-methoxy-9H-pyrimido[4,5-b]indol-4-yl)-3-fluoro-2-methylphenyl)ethyl)-1,2,4-oxadiazole-3-carboxamide

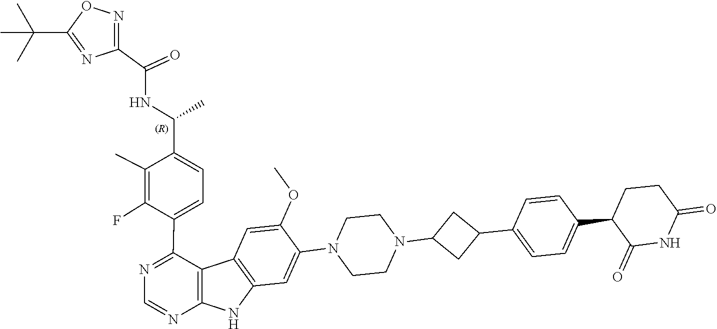

The titled compound was synthesized in the procedures similar to Example 47. ¹H NMR (500 MHz, DMSO) δ 12.17 (s, 1H), 10.82 (s, 1H), 9.60 (d, J=7.6 Hz, 1H), 8.89 (s, 1H), 7.58-7.51 (m, 2H), 7.25-7.13 (m, 4H), 7.01 (s, 1H), 6.73 (s, 1H), 5.44-5.37 (m, 1H), 3.82 (dd, J=11.2, 4.4 Hz, 1H), 3.57 (s, 3H), 3.19-3.04 (m, 4H), 2.83-2.75 (m, 1H), 2.68-2.63 (m, 2H), 2.60-2.56 (m, 3H), 2.48-2.35 (m, 8H), 2.21-2.13 (m, 1H), 2.07-1.99 (m, 1H), 1.91-1.82 (m, 2H), 1.56 (d, J=6.9 Hz, 3H), 1.43 (s, 9H); [M+H]⁺=828.7.

### Example 61: 5-(tert-butyl)-N-((1R)-1-(4-(7-(4-(1-(3-(2,6-dioxopiperidin-3-yl)phenyl)pyrrolidin-3-yl)piperazin-1-yl)-6-methoxy-9H-pyrimido[4,5-b]indol-4-yl)-3-fluoro-2-methylphenyl)ethyl)-1,2,4-oxadiazole-3-carboxamide

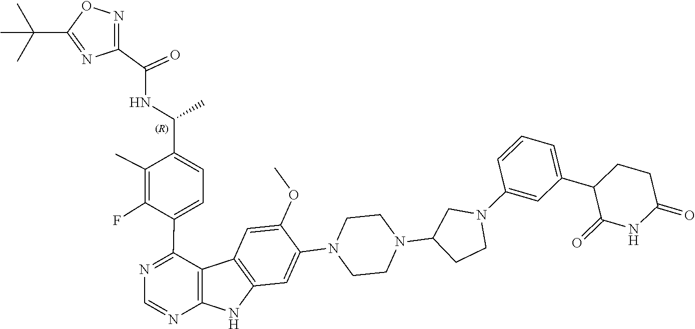

The titled compound was synthesized in the procedures similar to Example 47. ¹H NMR (500 MHz, MeOD) δ 8.84 (s, 1H), 7.53 (s, 2H), 7.17 (t, J=7.9 Hz, 2H), 6.82 (s, 1H), 6.57-6.51 (m, 2H), 6.47 (s, 1H), 5.54 (dd, J=13.0, 6.2 Hz, 1H), 3.80 (dd, J=9.3, 5.6 Hz, 1H), 3.68-3.55 (m, 4H), 3.51-3.43 (m, 1H), 3.27-3.10 (m, 6H), 2.93-2.76 (m, 4H), 2.71-2.57 (m, 3H), 2.51 (d, J=1.9 Hz, 3H), 2.36 (dt, J=12.0, 6.2 Hz, 1H), 2.29-2.19 (m, 2H), 1.99 (d, J=18.9, 9.4 Hz, 1H), 1.65 (d, J=7.0 Hz, 3H), 1.49 (s, 9H); [M+H]⁺=843.6.

### Example 62: (R)-3-(tert-butyl)-N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-5-fluoro-2-methylphenyl)ethyl)-1,2,4-oxadiazole-5-carboxamide

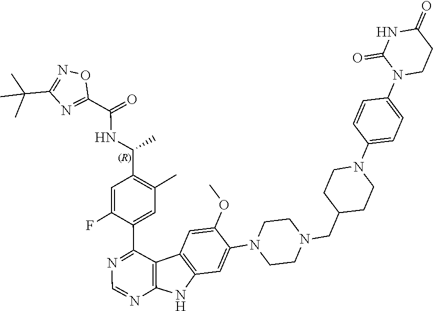

The titled compound was synthesized in the procedures similar to Example 47. ¹H NMR (500 MHz, DMSO) δ 12.12 (s, 1H), 10.19 (s, 1H), 9.48 (d, J=7.8 Hz, 1H), 8.82 (d, J=5.2 Hz, 1H), 7.48 (dd, J=9.3, 3.6 Hz, 2H), 7.06 (d, J=9.0 Hz, 2H), 6.95 (s, 1H), 6.86 (d, J=9.1 Hz, 2H), 6.72 (d, J=1.7 Hz, 1H), 5.30 (q, J=7.1 Hz, 1H), 3.62 (dd, J=12.8, 6.0 Hz, 4H), 3.49 (s, 3H), 3.00 (s, 4H), 2.62-2.56 (m, 5H), 2.44 (dt, J=3.6, 1.8 Hz, 3H), 2.41 (s, 3H), 2.17 (d, J=7.1 Hz, 2H), 1.75 (d, J=11.4 Hz, 2H), 1.68-1.59 (m, 1H), 1.47 (d, J=7.0 Hz, 3H), 1.37 (s, 9H), 1.19-1.12 (m, 2H); [M+H]⁺=872.5.

### Example 63: (R)-5-(tert-butyl)-N-(1-(4-(7-(4-(1-(4-(2,4-dioxotetrahydropyrimidin-1(2H)-yl)phenyl)azetidin-3-yl)piperazin-1-yl)-9H-pyrimido[4,5-b]indol-4-yl)-3-fluoro-2-methylphenyl)ethyl)-1,2,4-oxadiazole-3-carboxamide

### Step 1: 3,3′-((4-hydroxyphenyl)azanediyl)dipropionic acid

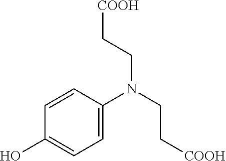 [SMILES: O=C(O)CCN(CCC(=O)O)c1ccc(O)cc1]

To a solution of 4-aminophenol (250 g, 2.29 mol) and acrylic acid (363 g, 5.04 mol) in water (1.25 L) was stirred at 80-85° C. for 16 hrs. The reaction mixture was filtered and the filter cake was washed with H₂O (2.00 L). The filtrate was concentrated in vacuum to give the titled product. (552 g, 95.1%). ¹H NMR (400 MHz, DMSO) δ 12.18 (brs, 2H), 8.67 (brs, 1H), 6.48-6.88 (m, 4H), 3.38 (s, 5H), 2.36 (t, J=7.06 Hz, 4H).

### Step 2: 1-(4-hydroxyphenyl)dihydropyrimidine-2,4(1H,3H)-dione

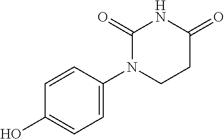 [SMILES: O=C1CCN(c2ccc(O)cc2)C(=O)N1]

To a solution of 3,3′-((4-hydroxyphenyl)azanediyl)dipropionic acid (300 g, 1.18 mol) in AcOH (1.50 L) was added urea (106 g, 1.76 mol) at 20-30° C. After addition, the mixture was stirred at 120-130° C. for 12 hrs. LCMS showed the starting material was consumed completely and the desired product was observed. 10.0% HCl (1.00 L) was added to the reactor at 120-130° C. After addition, the mixture was stirred at 120-130° C. for 1 hr. The reaction mixture was cooled to 10-20° C. and filtered. The filter cake was rinsed with petroleum ether (2.00 L) to give the titled product (185 g, crude). ¹H NMR (400 MHz, DMSO) δ 10.3 (s, 1H), 9.45 (s, 1H), 7.00-7.26 (m, 2H), 6.67-6.86 (m, 2H), 3.67 (t, J=6.72 Hz, 2H), 2.67 (t, J=6.72 Hz, 2H).

### Step 3: 1-(4-((tert-butyldimethylsilyl)oxy)phenyl)dihydropyrimidine-2,4(1H,3H)-dione

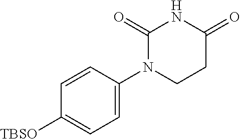 [SMILES: CC(C)(C)[Si](C)(C)Oc1ccc(N2CCC(=O)NC2=O)cc1]

To a solution of 1-(4-hydroxyphenyl)dihydropyrimidine-2,4(1H,3H)-dione (80.0 g, 0.380 mol) and imidazole (52.8 g, 0.770 mol) in DCM (400 mL) was added TBSCl (64.3 g, 0.420 mol) at 10-15° C. Then the mixture was stirred at 25° C. for 12 hrs. The reaction mixture was partitioned between DCM (400 mL) and water (500 mL). The aqueous layer was extracted with DCM (200 mL). The combined organic layers was washed with brine (200 mL), dried with anhydrous Na₂SO₄, and concentrated in vacuo to give the titled product (120 g, 96.1%). ¹H NMR (400 MHz, DMSO) δ 10.31 (s, 1H), 7.07-7.40 (m, 2H), 6.67-6.95 (m, 2H), 3.72 (t, J=6.73 Hz, 2H), 2.68 (t, J=6.73 Hz, 2H), 0.95 (s, 9H), 0.20 (s, 6H).

### Step 4: 1-(4-((tert-butyldimethylsilyl)oxy)phenyl)-3-((2-(trimethylsilyl)ethoxy)methyl)dihydropyrimidine-2,4(1H,3H)-dione

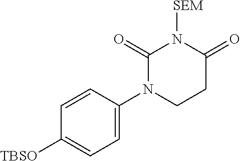 [SMILES: CC(C)(C)[Si](C)(C)Oc1ccc(N2CCC(=O)N(COCC[Si](C)(C)C)C2=O)cc1]

To a solution of 1-(4-((tert-butyldimethylsilyl)oxy)phenyl)dihydropyrimidine-2,4(1H,3H)-dione (85.0 g, 0.260 mol) and DIEA (137 g, 1.06 mol) in ACN (425 mL) was added SEMCl (88.4 g, 0.530 mol) at 20-25° C. Then the mixture was stirred at 80-85° C. for 12 hrs. TLC (petroleum ether:ethyl acetate=1:1, R_{f} (Starting material)=0.6, R_{f} (product)=0.9) indicated starting material was consumed. The reaction mixture was cooled to 20-25° C. and added into water (500 mL). The mixture was filtered and concentrated in reduced pressure at 45° C. The residue was purified by activated carbon, then adsorption by silica gel to give the product (124 g, 68.6%). ¹H NMR (400 MHz, CDCl₃) δ 7.03-7.21 (m, 2H), 6.85 (d, J=8.60 Hz, 2H), 5.29 (s, 2H), 3.78 (t, J=6.62 Hz, 2H), 3.61-3.72 (m, 2H), 2.89 (t, J=6.62 Hz, 2H), 0.98 (s, 9H), 0.72-0.93 (m, 1H), 0.20 (s, 5H), 0.03-0.04 (m, 10H).

### Step 5: 1-(4-hydroxyphenyl)-3-((2-(trimethylsilyl)ethoxy)methyl)dihydropyrimidine-2,4(1H,3H)-dione

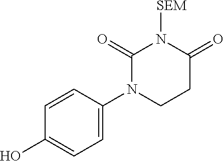 [SMILES: C[Si](C)(C)CCOCN1C(=O)CCN(c2ccc(O)cc2)C1=O]

To a solution of 1-(4-((tert-butyldimethylsilyl)oxy)phenyl)-3-((2-(trimethylsilyl)ethoxy)methyl)dihydropyrimidine-2,4(1H,3H)-dione (124 g, 0.270 mol) and NH₄F (20.3 g, 0.550 mol) in MeOH (620 mL) was stirred at 15-20° C. for 1 hr. The mixture was filtered and concentrated in reduced pressure at 45° C. The crude product was triturated with petroleum ether:ethyl acetate=10:1 (200 mL) at 15° C. for 45 mins, to give the target product (65.0 g, 68.6%). ¹H NMR (400 MHz, DMSO) δ 6.96-7.15 (m, 2H), 6.63-6.84 (m, 2H), 5.31 (s, 2H), 5.93 (s, 1H), 3.76 (t, J=6.80 Hz, 2H), 3.66-3.73 (m, 2H), 2.90 (t, J=6.58 Hz, 2H), 0.87-1.16 (m, 2H), 0.02 (s, 9H).

### Step 6: 4-(2,4-dioxo-3-((2-(trimethylsilyl)ethoxy)methyl)tetrahydropyrimidin-1(2H)-yl)phenyl trifluoromethanesulfonate

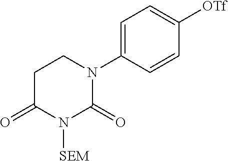 [SMILES: C[Si](C)(C)CCOCN1C(=O)CCN(c2ccc(OS(=O)(=O)C(F)(F)F)cc2)C1=O]

To a mixture of 1-(4-hydroxyphenyl)-3-((2-(trimethylsilyl)ethoxy)methyl)dihydropyrimidine-2,4(1H,3H)-dione (4 g, 11.9 mmol) and pyridine (1.88 g, 23.8 mmol) in DCM (30 mL) was added Tf₂O (4.03 g, 14.28 mmol) dropwise at 0° C. The mixture was stirred at rt for 3 h. The mixture was diluted with water (100 mL) and extracted with DCM (3×100 mL). The combined organic layers were dried over Na₂SO₄, filtered and concentrated under vacuum. The residue was purified by silica gel column chromatography, eluted with EA/PE (2:3) to afford the product (5.1 g, 91.5%). [M+H]⁺=469.1.

### Step 7: 1-(4-(3-hydroxyazetidin-1-yl)phenyl)-3-((2-(trimethylsilyl)ethoxy)methyl)dihydropyrimidine-2,4(1H,3H)-dione

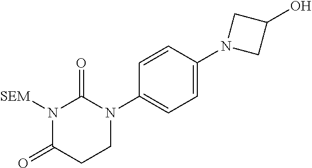 [SMILES: C[Si](C)(C)CCOCN1C(=O)CCN(c2ccc(N3CC(O)C3)cc2)C1=O]

A mixture of 4-(2,4-dioxo-3-((2-(trimethylsilyl)ethoxy)methyl)tetrahydropyrimidin-1(2H)-yl)phenyl trifluoromethanesulfonate (1.25 g, 0.00267 mol), azetidin-3-ol hydrochloride (582 mg, 0.00534 mol), Pd₂(dba)₃ (250 mg, 0.00267 mol), Ruphos (125 mg 0.00027 mol) and Cs₂CO₃ (2.6 g, 0.00810 mol) in 1,4-dioxane (20 mL) was stirred in a round bottom flask at 100° C. overnight under nitrogen. The mixture was evaporated in vacuum to afford the crude product, which was further purified with silica gel column chromatography (PE:EA=100:0˜0:100 gradient elution) to give the titled product (600 mg, 57%). [M+H]⁺=392.3.

### Step 8: 1-(4-(3-oxoazetidin-1-yl)phenyl)-3-((2-(trimethylsilyl)ethoxy)methyl)dihydropyrimidine-2,4(1H,3H)-dione

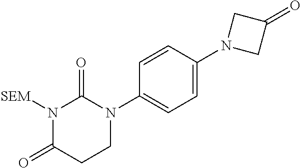 [SMILES: C[Si](C)(C)CCOCN1C(=O)CCN(c2ccc(N3CC(=O)C3)cc2)C1=O]

To a solution of (COCl)₂ (2.76 mL, 1.38 mmol, 1 M in DCM) in DCM (10 mL), DMSO (430 mg, 5.52 mmol, dissolved in 2 mL DCM) was added dropwise at −60° C. The mixture was stirred at −60° C. for 1 hour. Then 1-(4-(3-hydroxyazetidin-1-yl)phenyl)-3-((2-(trimethylsilyl)ethoxy)methyl)dihydropyrimidine-2,4(1H,3H)-dione (540 mg, 1.38 mmol, dissolved in 2 mL DCM) was added. The mixture was stirred at −60° C. for 1 hour. Then triethylamine (836 mg, 8.28 mmol) was added, and the mixture was stirred from −60° C. to room temperature. The mixture was evaporated in vacuum to afford the crude product, which was further purified with silica gel column chromatography (PE:EA=100:0˜90:10 gradient elution) to give the titled product (400 mg, 74%). [M+H]⁺=390.2.

### Step 9: (R)-5-(tert-butyl)-N-(1-(4-(7-(4-(1-(4-(2,4-dioxotetrahydropyrimidin-1(2H)-yl)phenyl)azetidin-3-yl)piperazin-1-yl)-9H-pyrimido[4,5-b]indol-4-yl)-3-fluoro-2-methylphenyl)ethyl)-1,2,4-oxadiazole-3-carboxamide

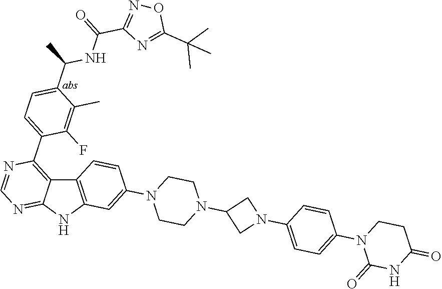 [SMILES: Cc1c([C@@H](C)NC(=O)c2noc(C(C)(C)C)n2)ccc(-c2ncnc3[nH]c4cc(N5CCN(C6CN(c7ccc(N8CCC(=O)NC8=O)cc7)C6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (4 mL) and MeOH (4 mL), NaOAc (44.3 mg, 0.540 mmol) was added. The mixture was stirred at room temperature for 5 mins. Then 1-(4-(3-oxoazetidin-1-yl)phenyl)-3-((2-(trimethylsilyl)ethoxy)methyl)dihydropyrimidine-2,4(1H,3H)-dione (84 mg, 0.216 mmol) and HOAc (0.06 mL) were added. The mixture was stirred at room temperature overnight. Then NaBH(OAc)₃ (190 mg, 0.9 mmol) was added and stirred at room temperature overnight. Then NaBH₃CN (114 mg, 1.8 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was diluted with DCM (3 mL), and TFA (3 mL) was added. The mixture was stirred at room temperature for 2 hours, and the mixture was concentrated in vacuo. The residue was dissolved in DCM (3 mL), and NH₃·H₂O (0.5 mL) was added. The mixture was stirred for 30 mins, concentrated under vacuum, and further purified by prep-HPLC to afford the desired product (2.48 mg, 1.6%). ¹H NMR (500 MHz, DMSO) δ 12.19 (s, 1H), 10.23 (s, 1H), 9.60 (d, J=10.0 Hz, 1H), 8.85 (s, 1H), 7.59-7.45 (m, 2H), 7.22 (s, 1H), 7.11 (d, J=10.0 Hz, 2H), 6.93 (s, 2H), 6.45 (d, J=5.0 Hz, 2H), 5.50-5.38 (m, 1H), 3.95 (s, 2H), 3.67 (s, 4H), 3.28-3.24 (m, 6H), 2.72-2.64 (m, 3H), 2.42-2.35 (m, 5H), 1.60-1.55 (m, 3H), 1.43 (s, 9H); [M+H]⁺=800.5.

### Example 64: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-3-fluoro-2-methylphenyl)ethyl)-3-(1-methylcyclopropyl)-1,2,4-oxadiazole-5-carboxamide

### Step 1: 1-(4-bromo-3-fluoro-2-methylphenyl)ethan-1-one

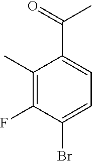

To a stirred solution of 1-bromo-2-fluoro-4-iodo-3-methylbenzene (25.00 g, 79.384 mmol) and tributyl(1-ethoxyethenyl)stannane (34.40 g, 95.261 mmol) in DMF (500 mL) was added Pd(PPh₃)₂Cl₂ (5.57 g, 7.938 mmol) at room temperature. The resulting mixture was stirred overnight at 80° C. under nitrogen atmosphere. The mixture was allowed to cool down to room temperature. The resulting mixture was diluted with water (800 mL) and extracted with PE (3×600 mL). The organic layer was washed with 2×200 mL of brine. The resulting mixture was concentrated under reduced pressure and diluted with THF (600 mL). The mixture was acidified to pH 3 with HCl (aq.). The resulting mixture was stirred for 1 h at room temperature, extracted with EtOAc (3×500 mL) and washed with 2×500 mL of brine. The organic layer was dried over anhydrous Na₂SO₄. After filtration, the filtrate was concentrated under reduced pressure. The residue was purified by silica gel column chromatography, eluted with PE/EtOAc (10:1) to afford 1-(4-bromo-3-fluoro-2-methylphenyl)ethanone (16.65 g, 90.8%). ¹H NMR (400 MHz, CDCl₃) δ 7.48 (ddd, J=8.4, 6.5, 0.7 Hz, 1H), 7.35 (dd, J=8.4, 1.3 Hz, 1H), 2.58 (s, 3H), 2.46 (d, J=2.7 Hz, 3H).

### Step 2: (R,E)-N-(1-(4-bromo-3-fluoro-2-methylphenyl)ethylidene)-2-methylpropane-2-sulfinamide

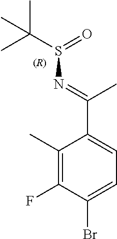

To a stirred solution of 1-(4-bromo-3-fluoro-2-methylphenyl)ethan-1-one (30.00 g, 129.834 mmol) and Ti(Oi-Pr)₄ (184.50 g, 649.171 mmol) in THF (600 mL) was added (R)-2-methylpropane-2-sulfinamide (39.34 g, 324.585 mmol) in portions at room temperature. The resulting mixture was stirred for 4 days at 80° C. The mixture was allowed to cool down to 15° C. The reaction was quenched with ice/water (400 mL) and EA (600 mL) at 15° C. The resulting mixture was filtered and the filter cake was washed with EtOAc (3×2 L). The filtrate was concentrated under reduced pressure. The resulting mixture was diluted with EtOAc (600 mL). The organic layer was washed with 2×300 mL of brine. The residue was purified by silica gel column chromatography, eluted with hexane/EtOAc (20:1) to afford the product (40.0 g, 83.8%). ¹H NMR (400 MHz, CDCl₃) δ 7.50-7.38 (m, 1H), 7.05-6.96 (m, 1H), 2.70 (s, 3H), 2.36 (d, J=2.7 Hz, 3H), 1.31 (s, 9H); [M+H]⁺=336.0.

### Step 3: (R)—N—((R)-1-(4-bromo-3-fluoro-2-methylphenyl)ethyl)-2-methylpropane-2-sulfinamide

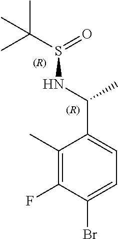

To a stirred solution of (R,E)-N-(1-(4-bromo-3-fluoro-2-methylphenyl)ethylidene)-2-methylpropane-2-sulfinamide (40.00 g, 119.671 mmol) in THF/MeOH (1/1, 800 mL) was added NaBH₄ (4.53 g, 119.671 mmol) in portions at −60° C. under nitrogen atmosphere. The resulting mixture was stirred overnight at room temperature under nitrogen atmosphere. The reaction was quenched with sat. NH₄Cl (aq.) at 0° C. The aqueous layer was extracted with CH₂Cl₂ (3×300 mL). The organic layer was washed with 2×200 mL of brine and dried over anhydrous Na₂SO₄. After filtration, the filtrate was concentrated under reduced pressure. The residue was purified by silica gel column chromatography, eluted with PE/EtOAc (50:1 to 10:1) to afford the product (27.3 g, 67.9%). ¹H NMR (400 MHz, CDCl₃) δ 7.43-7.35 (m, 1H), 7.08 (dd, J=8.4, 1.3 Hz, 1H), 4.80 (qd, J=6.7, 3.5 Hz, 1H), 3.29 (d, J=2.8 Hz, 1H), 2.35 (d, J=2.4 Hz, 3H), 1.51 (d, J=6.7 Hz, 3H), 1.22 (s, 9H); [M+H]⁺ 336.0.

### Step 4: (R)—N—((R)-1-(3-fluoro-2-methyl-4-(4,4,5,5-tetramethyl-1,3,2-dioxaborolan-2-yl)phenyl)ethyl)-2-methylpropane-2-sulfinamide

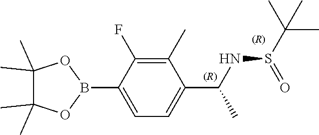

To a stirred mixture of (R)—N—((R)-1-(4-bromo-3-fluoro-2-methylphenyl)ethyl)-2-methylpropane-2-sulfinamide (14.00 g, 41.634 mmol) and 4,4,5,5-tetramethyl-2-(tetramethyl-1,3,2-dioxaborolan-2-yl)-1,3,2-dioxaborolane (21.15 g, 83.269 mmol) in 1,4-dioxane (280 mL) were added KOAc (8.17 g, 83.269 mmol) and Pd(dppf)Cl₂ (1.83 g, 2.498 mmol) in portions at room temperature. The resulting mixture was stirred overnight at 80° C. under nitrogen atmosphere and concentrated under vacuum. The residue was purified by silica gel column chromatography, eluted with PE/EtOAc (3:1) to afford the product (11.95 g, 74.89%). ¹H NMR (400 MHz, DMSO) δ 7.50-7.38 (m, 1H), 7.24 (d, J=7.8 Hz, 1H), 5.40 (d, J=5.2 Hz, 1H), 4.68-4.55 (m, 1H), 2.19 (d, J=2.3 Hz, 3H), 1.42 (d, J=6.7 Hz, 3H), 1.28 (s, 12H), 1.09 (s, 9H); [M+H]⁺=384.1.

### Step 5: (R)-1-(3-fluoro-2-methyl-4-(4,4,5,5-tetramethyl-1,3,2-dioxaborolan-2-yl)phenyl)ethan-1-amine hydrochloride

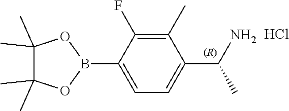

To a stirred solution of (R)—N—((R)-1-(3-fluoro-2-methyl-4-(4,4,5,5-tetramethyl-1,3,2-dioxaborolan-2-yl)phenyl)ethyl)-2-methylpropane-2-sulfinamide (21.70 g, 56.609 mmol) in DCM (200 mL) was added 4 M HCl in 1,4-dioxane (200.00 mL) dropwise at room temperature. The resulting mixture was stirred for 1 h at room temperature. The resulting mixture was concentrated under reduced pressure and diluted with MTBE (450 mL). The precipitated solids were collected by filtration and washed with MTBE (2×200 mL) and Et₂O (2×300 mL) to give the product (12.2 g, 68.50%). ¹H NMR (400 MHz, DMSO) δ 8.63 (s, 3H), 7.52 (dd, J=7.8, 6.0 Hz, 1H), 7.43 (d, J=7.9 Hz, 1H), 4.57 (q, J=6.7 Hz, 1H), 2.23 (d, J=2.2 Hz, 3H), 1.46 (d, J=6.8 Hz, 3H), 1.29 (s, 12H); [M+H]⁺=280.1.

### Step 6: (R)—N-(1-(3-fluoro-2-methyl-4-(4,4,5,5-tetramethyl-1,3,2-dioxaborolan-2-yl)phenyl)ethyl)-3-(1-methylcyclopropyl)-1,2,4-oxadiazole-5-carboxamide

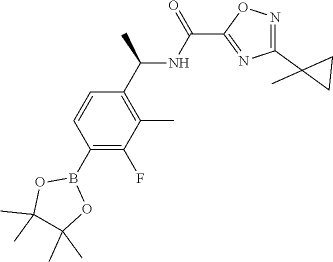 [SMILES: Cc1c([C@@H](C)NC(=O)c2nc(C3(C)CC3)no2)ccc(B2OC(C)(C)C(C)(C)O2)c1F]

To a solution of (R)-1-(3-fluoro-2-methyl-4-(4,4,5,5-tetramethyl-1,3,2-dioxaborolan-2-yl)phenyl)ethan-1-amine hydrochloride (1.5 g, 4.7 mmol) in DCM (20 mL), NH₃ (7 M in MeOH, 3 mL) was added. The mixture was stirred at room temperature for 20 mins and extracted with DCM (2*20 mL). The organic layer was combined, dried over Na₂SO₄, and concentrated in vacuo. The residue was diluted with EtOH (10 mL) and ethyl 3-(1-methylcyclopropyl)-1,2,4-oxadiazole-5-carboxylate (0.924 g, 0.0047 mol) was added. The mixture was stirred under reflux overnight. The mixture was concentrated in vacuo, and the residue was purified with silica gel column chromatography (PE:EA=100:0˜0:100 gradient elution) to give the titled product (950 mg, 48%). [M+H]⁺=430.2.

### Step 6: benzyl (R)-4-(4-(2-fluoro-3-methyl-4-(1-(3-(1-methylcyclopropyl)-1,2,4-oxadiazole-5-carboxamido)ethyl)phenyl)-9H-pyrimido[4,5-b]indol-7-yl)piperazine-1-carboxylate

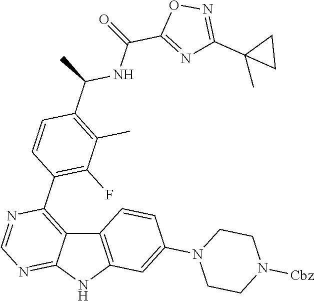 [SMILES: Cc1c([C@@H](C)NC(=O)c2nc(C3(C)CC3)no2)ccc(-c2ncnc3[nH]c4cc(N5CCN(C(=O)OCc6ccccc6)CC5)ccc4c23)c1F]

A mixture of benzyl 4-(4-chloro-9H-pyrimido[4,5-b]indol-7-yl)piperazine-1-carboxylate (930 mg, 2.21 mmol), (R)—N-(1-(3-fluoro-2-methyl-4-(4,4,5,5-tetramethyl-1,3,2-dioxaborolan-2-yl)phenyl)ethyl)-3-(1-methylcyclopropyl)-1,2,4-oxadiazole-5-carboxamide (950 mg, 2.21 mmol), Pd(dppf)Cl₂ (160 mg, 0.22 mmol) and Cs₂CO₃ (1.29 g, 3.99 mmol) in 1,4-dioxane (20 mL) and water (4 mL) was stirred at 93° C. overnight. The mixture was evaporated in vacuum to afford the crude product, which was further purified with silica gel column chromatography (PE:EA=100:0˜0:100 gradient elution) to give the titled product (830 mg, 54%). [M+H]⁺=689.6.

### Step 7: (R)—N-(1-(3-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide

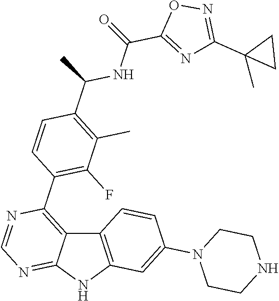 [SMILES: Cc1c([C@@H](C)NC(=O)c2nc(C3(C)CC3)no2)ccc(-c2ncnc3[nH]c4cc(N5CCNCC5)ccc4c23)c1F]

A mixture of benzyl (R)-4-(4-(2-fluoro-3-methyl-4-(1-(3-(1-methylcyclopropyl)-1,2,4-oxadiazole-5-carboxamido)ethyl)phenyl)-9H-pyrimido[4,5-b]indol-7-yl)piperazine-1-carboxylate (830 mg, 1.2 mmol) in trifluoroacetic acid (10 mL) was stirred at 85° C. under reflux for 2 hours. The mixture was evaporated in vacuum to afford the product (1.8 g, crude), which was used in next step without further purification. [M+H]⁺=555.6.

### Step 8: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-3-fluoro-2-methylphenyl)ethyl)-3-(1-methylcyclopropyl)-1,2,4-oxadiazole-5-carboxamide

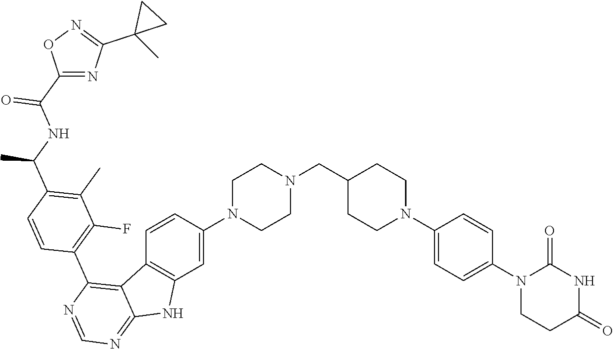 [SMILES: Cc1c([C@@H](C)NC(=O)c2nc(C3(C)CC3)no2)ccc(-c2ncnc3[nH]c4cc(N5CCN(CC6CCN(c7ccc(N8CCC(=O)NC8=O)cc7)CC6)CC5)ccc4c23)c1F]

To a solution of (R)—N-(1-(3-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (140 mg, crude) in dichloromethane (4 mL) and MeOH (4 mL), NaOAc (62 mg, 0.758 mmol) was added. The mixture was stirred at room temperature for 5 mins. Then 1-(4-(2,4-dioxotetrahydropyrimidin-1(2H)-yl)phenyl)piperidine-4-carbaldehyde (76 mg, 0.252 mmol) and HOAc (0.06 mL) were added. The mixture was stirred at room temperature overnight. Then NaBH(OAc)₃ (267 mg, 1.264 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give crude product, which was further purified by prep-TLC (DCM:MeOH=10:1) to afford the desired product (43.91 mg, 20%). ¹H NMR (500 MHz, DMSO) δ 12.20 (s, 1H), 10.25 (s, 1H), 9.92 (d, J=5.0 Hz, 1H), 8.85 (s, 1H), 7.60-7.46 (m, 2H), 7.21 (d, J=5.0 Hz, 1H), 7.13 (d, J=5.0 Hz, 2H), 6.93 (d, J=5.0 Hz, 4H), 5.51-5.19 (m, 1H), 3.73-3.60 (m, 4H), 3.24 (s, 5H), 2.75-2.61 (m, 5H), 2.57-2.51 (m, 3H), 2.39 (s, 4H), 2.23 (s, 2H), 1.88-1.66 (m, 3H), 1.57 (d, J=10.0 Hz, 3H), 1.49 (s, 3H), 1.21 (s, 2H), 0.99 (s, 2H); [M+H]⁺=840.6.

### Example 65: N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-3-fluoro-2-methylphenyl)ethyl)-3-(1-methylcyclopropyl)-1,2,4-oxadiazole-5-carboxamide

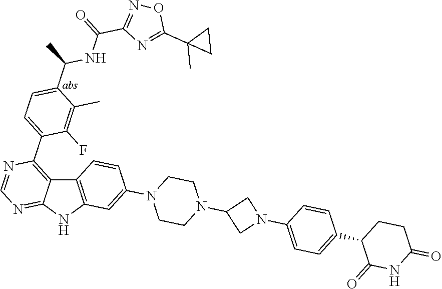 [SMILES: Cc1c([C@@H](C)NC(=O)c2noc(C3(C)CC3)n2)ccc(-c2ncnc3[nH]c4cc(N5CCN(C6CN(c7ccc([C@@H]8CCC(=O)NC8=O)cc7)C6)CC5)ccc4c23)c1F]

To a solution of (R)—N-(1-(3-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (140 mg, crude) in dichloromethane (4 mL) and MeOH (4 mL), NaOAc (62 mg, 0.758 mmol) was added. The mixture was stirred at room temperature for 5 mins. Then (R)-3-(4-(3-oxocyclobutyl)phenyl)piperidine-2,6-dione (64 mg, 0.252 mmol) and HOAc (0.06 mL) was added. The mixture was stirred at room temperature overnight. Then NaBH(OAc)₃ (267 mg, 1.264 mmol) was added and stirred at room temperature overnight. Then NaBH₃CN (164 mg, 2.52 mmol) was added and stirred at room temperature overnight. The reaction mixture was concentrated in vacuum, and the residue was purified by silica gel column chromatography (DCM:MeOH=100%: 0%˜90%: 10% gradient elution) to give crude product, which was further purified by prep-TLC (DCM:MeOH=10:1) to afford the desired product (23.98 mg, 11%). ¹H NMR (500 MHz, DMSO) δ 12.19 (s, 1H), 10.81 (s, 1H), 9.92 (d, J=10.0 Hz, 1H), 8.85 (s, 1H), 7.58-7.47 (m, 2H), 7.32-7.10 (m, 5H), 6.96-6.87 (m, 2H), 5.50-5.29 (m, 1H), 3.82 (dd, J=10.0, 5.0 Hz, 1H), 3.30-3.21 (m, 5H), 3.20-3.08 (m, 1H), 2.69-2.60 (m, 3H), 2.59-2.54 (m, 3H), 2.41-2.34 (m, 4H), 2.25-2.12 (m, 2H), 2.07-1.99 (m, 1H), 1.90 (d, J=10.0 Hz, 1H), 1.57 (d, J=6.9 Hz, 3H), 1.49 (s, 3H), 1.24-1.18 (m, 2H), 0.99 (d, J=2.6 Hz, 2H); [M+H]⁺=796.5.

### Example 66: N—((R)-1-(4-(7-(4-(3-(4-((R)-2,6-dioxopiperidin-3-yl)phenyl)cyclobutyl)piperazin-1-yl)-9H-pyrimido[4,5-b]indol-4-yl)-3-fluoro-2-methylphenyl)ethyl)-3-(1-methylcyclopropyl)-1,2,4-oxadiazole-5-carboxamide

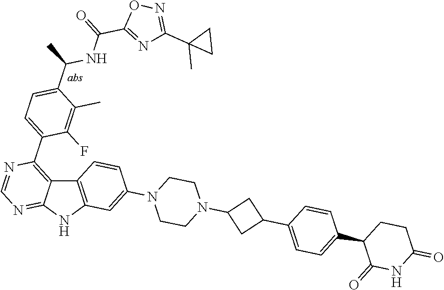 [SMILES: Cc1c([C@@H](C)NC(=O)c2nc(C3(C)CC3)no2)ccc(-c2ncnc3[nH]c4cc(N5CCN(C6CC(c7ccc([C@H]8CCC(=O)NC8=O)cc7)C6)CC5)ccc4c23)c1F]

To a solution of (R)—N-(1-(3-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (140 mg, crude) in dichloromethane (4 mL) and MeOH (4 mL), NaOAc (62 mg, 0.758 mmol) was added. The mixture was stirred at room temperature for 5 mins. Then (S)-3-(4-(3-oxocyclobutyl)phenyl)piperidine-2,6-dione (64 mg, 0.252 mmol) and HOAc (0.06 mL) was added. The mixture was stirred at room temperature overnight. Then NaBH(OAc)₃ (267 mg, 1.264 mmol) was added and stirred at room temperature overnight. Then NaBH₃CN (164 mg, 2.52 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (15.95 mg, 8%). ¹H NMR (500 MHz, DMSO) δ 12.19 (s, 1H), 10.81 (s, 1H), 9.92 (d, J=10.0 Hz, 1H), 8.85 (s, 1H), 7.58-7.44 (m, 2H), 7.30-7.09 (m, 5H), 6.98-6.84 (m, 2H), 5.42 (dd, J=14.3, 7.1 Hz, 1H), 3.82 (dd, J=10.0, 5.0 Hz, 1H), 3.25 (s, 5H), 3.19-3.09 (m, 1H), 2.80-2.72 (m, 1H), 2.70-2.59 (m, 1H), 2.48-2.45 (m, 4H), 2.41-2.34 (m, 5H), 2.23-2.13 (m, 2H), 2.05-1.98 (m, 1H), 1.94-1.85 (m, 1H), 1.57 (d, J=6.9 Hz, 3H), 1.49 (s, 3H), 1.23-1.18 (m, 2H), 0.99 (d, J=2.5 Hz, 2H); [M+H]⁺=796.5.

### Example 67: N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

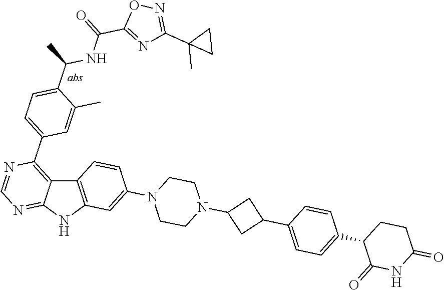 [SMILES: Cc1cc(-c2ncnc3[nH]c4cc(N5CCN(C6CC(c7ccc([C@@H]8CCC(=O)NC8=O)cc7)C6)CC5)ccc4c23)ccc1[C@@H](C)NC(=O)c1nc(C2(C)CC2)no1]

To a solution of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (120 mg, crude) in dichloromethane (4 mL) and MeOH (4 mL), NaOAc (55 mg, 0.672 mmol) was added. The mixture was stirred at room temperature for 5 mins. Then (R)-3-(4-(3-oxocyclobutyl)phenyl)piperidine-2,6-dione (69 mg, 0.268 mmol) and HOAc (0.06 mL) was added. The mixture was stirred at room temperature overnight. Then NaBH(OAc)₃ (237 mg, 1.12 mmol) was added and stirred at room temperature overnight. Then NaBH₃CN (143 mg, 2.24 mmol) was added and stirred at room temperature overnight. The reaction mixture was concentrated in vacuum, and the residue was purified by silica gel column chromatography (DCM:MeOH=100%: 0% 90%: 10% gradient elution) to give crude product, which was further purified by prep-HPLC to afford the desired product (40.14 mg, 23%). ¹H NMR (400 MHz, DMSO) δ 12.76 (s, 1H), 10.35 (s, 1H), 9.99 (s, 1H), 8.88 (s, 1H), 8.15 (s, 2H), 8.06 (d, J=6.8 Hz, 2H), 7.55 (s, 1H), 7.45 (s, 3H), 7.21 (d, J=7.2 Hz, 2H), 7.01 (d, J=7.8 Hz, 2H), 4.64 (s, 2H), 3.77 (d, J=5.6 Hz, 4H), 3.41 (s, 2H), 3.04 (s, 2H), 2.76-2.74 (m, 4H), 2.53-2.42 (m, 3H), 2.28 (s, 3H), 2.08 (s, 2H), 1.93-1.70 (m, 8H), 1.45 (s, 9H), 1.35-1.20 (m, 3H); [M+H]⁺=778.6.

### Example 68: N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

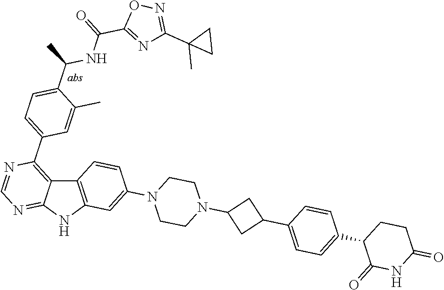 [SMILES: Cc1cc(-c2ncnc3[nH]c4cc(N5CCN(C6CC(c7ccc([C@@H]8CCC(=O)NC8=O)cc7)C6)CC5)ccc4c23)ccc1[C@@H](C)NC(=O)c1nc(C2(C)CC2)no1]

To a solution of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (120 mg, crude) in dichloromethane (4 mL) and MeOH (4 mL), NaOAc (55 mg, 0.672 mmol) was added. The mixture was stirred at room temperature for 5 mins. Then (S)-3-(4-(3-oxocyclobutyl)phenyl)piperidine-2,6-dione (69 mg, 0.268 mmol) and HOAc (0.06 mL) was added. The mixture was stirred at room temperature overnight. Then NaBH(OAc)₃ (237 mg, 1.12 mmol) was added and stirred at room temperature overnight. Then NaBH₃CN (143 mg, 2.24 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-HPLC to afford the desired product (42 mg, 24%). ¹H NMR (500 MHz, DMSO) δ 12.09 (s, 1H), 10.75 (s, 1H), 9.79 (d, J=10.0 Hz, 1H), 8.75 (s, 1H), 7.76-7.54 (m, 4H), 7.23-7.01 (m, 4H), 6.87 (s, 2H), 5.38-5.28 (m, 1H), 3.75 (dd, J=10.0, 5.0 Hz, 1H), 3.19 (s, 5H), 3.10-3.04 (m, 2H), 2.73-2.64 (m, 1H), 2.63-2.58 (m, 2H), 2.29 (s, 3H), 2.15-2.03 (m, 3H), 2.00-1.90 (m, 2H), 1.86-1.75 (m, 3H), 1.49 (d, J=6.9 Hz, 3H), 1.42 (s, 3H), 1.15-1.12 (m, 1H), 0.92 (d, J=2.6 Hz, 2H); [M+H]⁺=778.6.

### Example 69: 3-(tert-butyl)-N-((1R)-1-(4-(7-(4-((1-(2,6-dioxopiperidin-3-yl)-3-methyl-2-oxo-2,3-dihydro-1H-benzo[d]imidazol-5-yl)methyl)piperazin-1-yl)-6-methoxy-9H-pyrimido[4,5-b]indol-4-yl)-2-methylphenyl)ethyl)-1,2,4-oxadiazole-5-carboxamide

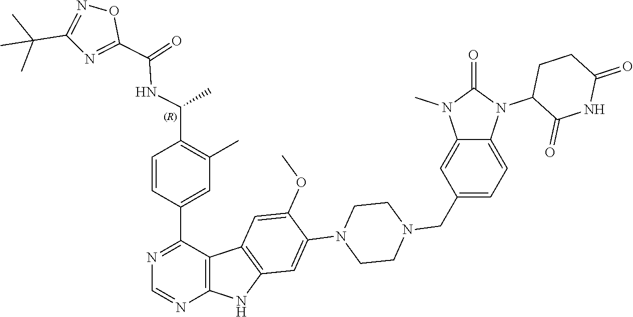

The titled compound was synthesized in the procedures similar to Example 47. ¹H NMR (500 MHz, DMSO) δ 12.14 (s, 1H), 11.10 (s, 1H), 9.93 (d, J=7.7 Hz, 1H), 8.85 (s, 1H), 7.81-7.75 (m, 2H), 7.72 (d, J=8.0 Hz, 1H), 7.27 (s, 1H), 7.17 (s, 1H), 7.08 (s, 1H), 7.04 (s, 1H), 7.01 (s, 1H), 5.43-5.33 (m, 2H), 3.65 (s, 3H), 3.57 (s, 1H), 3.36 (s, 3H), 3.29 (s, 2H), 3.16-3.00 (m, 3H), 2.96-2.86 (m, 1H), 2.75-2.70 (m, 1H), 2.65-2.54 (m, 4H), 2.52 (s, 3H), 2.05-1.99 (m, 1H), 1.57 (d, J=6.9 Hz, 3H), 1.37 (s, 9H); [M+H]⁺=840.5.

### Example 70: 3-(tert-butyl)-N-((1-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)piperidin-4-yl)methyl)-1,2,4-oxadiazole-5-carboxamide

### Step 1: tert-butyl 4-((3-(tert-butyl)-1,2,4-oxadiazole-5-carboxamido)methyl)piperidine-1-carboxylate

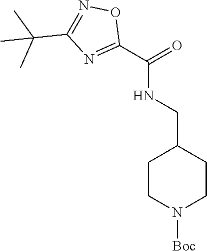 [SMILES: CC(C)(C)OC(=O)N1CCC(CNC(=O)c2nc(C(C)(C)C)no2)CC1]

To a suspension of sodium 3-(tert-butyl)-1,2,4-oxadiazole-5-carboxylate (1 g, 0.00520 mol) in DCM (20 mL), DMF (0.04 mL) was added. Then (COCl)₂ (10 mL, 0.010 mol, 1 M in DCM) was added dropwise slowly at 0° C. The mixture was stirred at room temperature for 3 hours. Then the mixture was concentrated in vacuo and diluted with DCM (10 mL). To a solution of tert-butyl 4-(aminomethyl)piperidine-1-carboxylate (1.11 g, 0.00520 mol) in DCM (40 mL), the mixture described above was added. Then TEA (2.6 g, 0.026 mmol) was added slowly. The mixture was stirred at room temperature overnight. The mixture was evaporated in vacuum to afford the crude product, which was further purified with silica gel column chromatography (PE:EA=100:0˜50:50 gradient elution) to give the titled product (940 mg, 50%). [M+Na]⁺=389.3.

### Step 2: 3-(tert-butyl)-N-(piperidin-4-ylmethyl)-1,2,4-oxadiazole-5-carboxamide hydrochloride

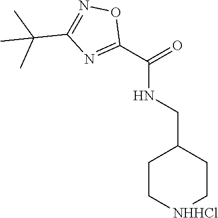

A mixture of tert-butyl 4-((3-(tert-butyl)-1,2,4-oxadiazole-5-carboxamido)methyl)piperidine-1-carboxylate (940 mg, 2.57 mmol) in 4 M HCl in dioxane (10 mL) was stirred at room temperature for 2.5 hours. Then the mixture was concentrated in vacuo to give the product (850 mg, crude), which was used in next step without further purification. [M+H]⁺=267.2.

### Step 3: benzyl 4-(4-(4-((3-(tert-butyl)-1,2,4-oxadiazole-5-carboxamido)methyl)piperidin-1-yl)-9H-pyrimido[4,5-b]indol-7-yl)piperazine-1-carboxylate

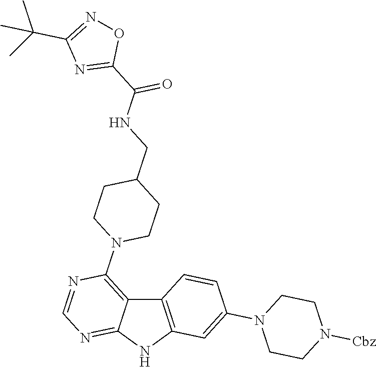 [SMILES: CC(C)(C)c1noc(C(=O)NCC2CCN(c3ncnc4[nH]c5cc(N6CCN(C(=O)OCc7ccccc7)CC6)ccc5c34)CC2)n1]

A mixture of 3-(tert-butyl)-N-(piperidin-4-ylmethyl)-1,2,4-oxadiazole-5-carboxamide hydrochloride (100 mg, 0.376 mmol), benzyl 4-(4-chloro-9H-pyrimido[4,5-b]indol-7-yl)piperazine-1-carboxylate (105 mg, 0.250 mmol) and DIEA (161 mg, 1.25 mmol) in i-PrOH (10 mL) was stirred under reflux overnight. Then the mixture was allowed to cool and concentrated in vacuo. The residue was purified by silica gel column chromatography (PE:EA=100%:0%˜0%:100%) to give the target product (80 mg, 50%). [M+H]⁺=652.2.

### Step 4: 3-(tert-butyl)-N-((1-(7-(piperazin-1-yl)-9H-pyrimido[4,5-b]indol-4-yl)piperidin-4-yl)methyl)-1,2,4-oxadiazole-5-carboxamide

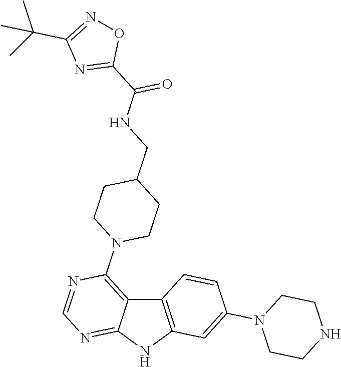 [SMILES: CC(C)(C)c1noc(C(=O)NCC2CCN(c3ncnc4[nH]c5cc(N6CCNCC6)ccc5c34)CC2)n1]

A solution of benzyl 4-(4-(4-((3-(tert-butyl)-1,2,4-oxadiazole-5-carboxamido)methyl)piperidin-1-yl)-9H-pyrimido[4,5-b]indol-7-yl)piperazine-1-carboxylate (80 mg, 0.123 mmol) in TFA (10 mL) was stirred under reflux for 3 hours. Then the mixture was concentrated in vacuo to give the product (143 mg, crude), which was used in next step without further purification. [M+H]⁺=518.4.

### Step 5: 3-(tert-butyl)-N-((1-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)piperidin-4-yl)methyl)-1,2,4-oxadiazole-5-carboxamide

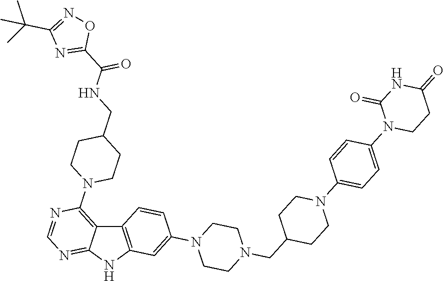 [SMILES: CC(C)(C)c1noc(C(=O)NCC2CCN(c3ncnc4[nH]c5cc(N6CCN(CC7CCN(c8ccc(N9CCC(=O)NC9=O)cc8)CC7)CC6)ccc5c34)CC2)n1]

To a solution of 3-(tert-butyl)-N-((1-(7-(piperazin-1-yl)-9H-pyrimido[4,5-b]indol-4-yl)piperidin-4-yl)methyl)-1,2,4-oxadiazole-5-carboxamide (140 mg, crude) in dichloromethane (4 mL) and MeOH (4 mL), NaOAc (66 mg, 0.810 mmol) was added. The mixture was stirred at room temperature for 5 mins. Then 1-(4-(2,4-dioxotetrahydropyrimidin-1(2H)-yl)phenyl)piperidine-4-carbaldehyde (105 mg, 0.352 mmol) and HOAc (0.06 mL) was added. The mixture was stirred at room temperature overnight. Then NaBH(OAc)₃ (286 mg, 1.35 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (77.95 mg, 36%). ¹H NMR (500 MHz, DMSO) δ 11.82 (s, 1H), 10.25 (s, 1H), 9.42 (t, J=5.0 Hz, 1H), 8.36 (s, 1H), 7.53 (d, J=5.0 Hz, 1H), 7.14 (d, J=10.0 Hz, 2H), 7.01 (d, J=5.0 Hz, 1H), 6.93 (d, J=5.0 Hz, 3H), 4.21 (d, J=15.0 Hz, 2H), 3.69 (t, J=6.5 Hz, 4H), 3.31-3.13 (m, 7H), 2.98 (t, J=11.0 Hz, 2H), 2.73-2.65 (m, 4H), 2.55 (s, 3H), 2.51 (s, 2H), 2.50-2.48 (m, 2H), 2.24 (s, 1H), 2.00-1.68 (m, 7H), 1.46-1.38 (m, 2H), 1.36 (s, 9H), 1.28-1.21 (m, 2H). [M+H]⁺=803.6.

### Example 71: 3-(tert-butyl)-N-((1R)-1-(4-(7-(4-((1-(2,6-dioxopiperidin-3-yl)-3-methyl-2-oxo-2,3-dihydro-1H-benzo[d]imidazol-5-yl)methyl)piperazin-1-yl)-6-methoxy-9H-pyrimido[4,5-b]indol-4-yl)-3-fluoro-2-methylphenyl)ethyl)-1,2,4-oxadiazole-5-carboxamide

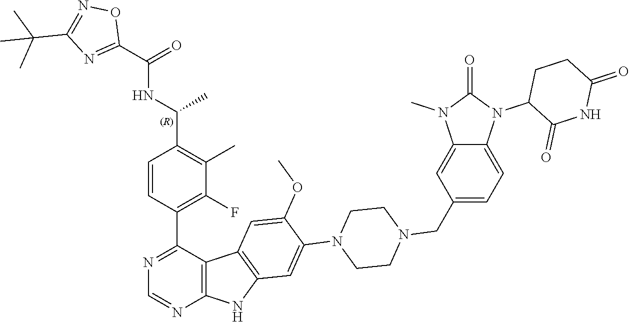

The titled compound was synthesized in the procedures similar to Example 47. ¹H NMR (500 MHz, DMSO) δ 12.19 (s, 1H), 11.10 (s, 1H), 10.00 (d, J=7.5 Hz, 1H), 8.88 (s, 1H), 8.38 (s, 3H), 7.55 (q, J=8.1 Hz, 2H), 7.15 (s, 1H), 7.07 (d, J=8.1 Hz, 1H), 7.01 (t, J=8.4 Hz, 2H), 6.71 (s, 1H), 5.46-5.31 (m, 2H), 3.59-3.52 (m, 4H), 3.35-3.26 (m, 5H), 3.06 (s, 3H), 2.95-2.86 (m, 1H), 2.74-2.67 (m, 1H), 2.66-2.53 (m, 4H), 2.41 (d, J=1.7 Hz, 3H), 2.05-1.99 (m, 1H), 1.58 (d, J=7.0 Hz, 3H), 1.37 (s, 9H); [M+H]⁺=858.6.

### Example 72: N—((R)-1-(4-(7-(4-(3-(4-((R)-2,6-dioxopiperidin-3-yl)phenyl)cyclobutyl)piperazin-1-yl)-6-methoxy-9H-pyrimido[4,5-b]indol-4-yl)-3-fluoro-2-methylphenyl)ethyl)-3-(1-methylcyclopropyl)-1,2,4-oxadiazole-5-carboxamide

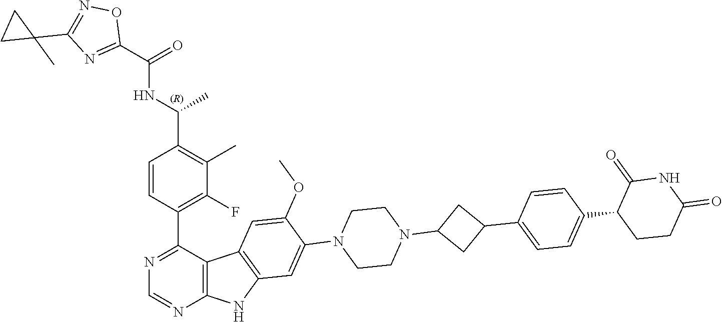

The titled compound was synthesized in the procedures similar to Example 47. ¹H NMR (500 MHz, DMSO) δ 12.17 (s, 1H), 10.81 (s, 1H), 9.93 (d, J=7.6 Hz, 1H), 8.88 (s, 1H), 7.57-7.48 (m, 2H), 7.22-7.13 (m, 4H), 7.01 (s, 1H), 6.72 (s, 1H), 5.38 (q, J=8.9 Hz, 1H), 3.82 (dd, J=11.2, 4.9 Hz, 1H), 3.56 (s, 3H), 3.15-3.07 (m, 4H), 2.78-2.74 (m, 1H), 2.70-2.62 (m, 2H), 2.40 (s, 3H), 2.17 (dd, J=18.9, 10.2 Hz, 2H), 2.06-1.99 (m, 1H), 1.87 (d, J=19.0 Hz, 5H), 1.75 (s, 3H), 1.57 (d, J=6.9 Hz, 3H), 1.49 (s, 3H), 1.20 (d, J=2.3 Hz, 2H), 0.99 (d, J=2.3 Hz, 2H); [M+H]⁺=826.4.

### Example 73: 3-(tert-butyl)-N-((1R)-1-(4-(7-(4-((1-(2,6-dioxopiperidin-3-yl)-3,3-dimethyl-2-oxoindolin-5-yl)methyl)piperazin-1-yl)-6-methoxy-9H-pyrimido[4,5-b]indol-4-yl)-2-methylphenyl)ethyl)-1,2,4-oxadiazole-5-carboxamide

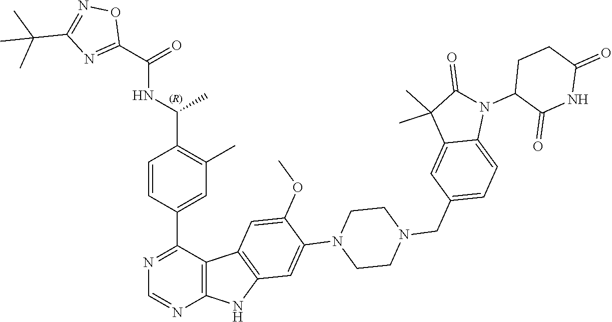

The titled compound was synthesized in the procedures similar to Example 47. ¹H NMR (500 MHz, DMSO) δ 12.33 (s, 1H), 11.15 (s, 1H), 10.01 (d, J=7.6 Hz, 1H), 8.92 (s, 1H), 7.88-7.75 (m, 4H), 7.34 (s, 2H), 7.10 (s, 2H), 5.46-5.42 (m, 1H), 5.37-5.32 (m, 1H), 4.45-4.38 (m, 1H), 3.71 (s, 4H), 3.61-3.51 (m, 1H), 3.45-3.34 (m, 1H), 3.21-3.16 (m, 4H), 3.03-2.88 (m, 1H), 2.75-2.58 (m, 7H), 2.03 (d, J=5.5 Hz, 1H), 1.63 (d, J=6.8 Hz, 3H), 1.40 (d, J=25.5 Hz, 15H); [M+H]⁺=853.5.

### Example 74: N-((1R)-1-(4-(7-(4-((1-(2,6-dioxopiperidin-3-yl)-3,3-dimethyl-2-oxoindolin-5-yl)methyl)piperazin-1-yl)-6-methoxy-9H-pyrimido[4,5-b]indol-4-yl)-2-methylphenyl)ethyl)-3-(1-methylcyclopropyl)-1,2,4-oxadiazole-5-carboxamide

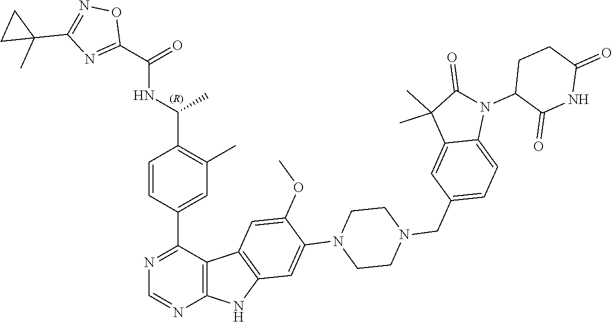

The titled compound was synthesized in the procedures similar to Example 47. ¹H NMR (500 MHz, DMSO) δ 12.18 (s, 1H), 11.08 (s, 1H), 9.87 (d, J=7.6 Hz, 1H), 8.85 (s, 1H), 7.80-7.74 (m, 2H), 7.70 (d, J=7.9 Hz, 1H), 7.34 (s, 1H), 7.27 (s, 1H), 7.20 (s, 1H), 7.02 (s, 1H), 6.96 (s, 1H), 5.37 (q, J=7.0 Hz, 1H), 5.24 (s, 1H), 3.65 (s, 3H), 3.51 (s, 2H), 3.13-3.07 (m, 4H), 2.89 (t, J=13.9 Hz, 1H), 2.74-2.53 (m, 7H), 1.97 (d, J=6.0 Hz, 1H), 1.56 (d, J=6.9 Hz, 3H), 1.49 (s, 3H), 1.32 (s, 6H), 1.25-1.18 (m, 4H), 0.99 (d, J=2.4 Hz, 2H); [M+H]⁺=851.6.

### Example 75: 3-(tert-butyl)-N-((1R)-1-(4-(7-(4-(4-((2,6-dioxopiperidin-3-yl)thio)benzyl)piperazin-1-yl)-6-methoxy-9H-pyrimido[4,5-b]indol-4-yl)-2-methylphenyl)ethyl)-1,2,4-oxadiazole-5-carboxamide

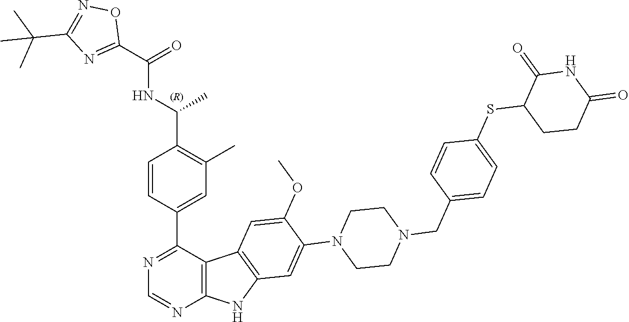

The titled compound was synthesized in the procedures similar to Example 47. ¹H NMR (500 MHz, DMSO) δ 12.12 (s, 1H), 10.88 (s, 1H), 9.87 (s, 1H), 8.79 (s, 1H), 7.81-7.61 (m, 3H), 7.58-7.35 (m, 3H), 7.34-7.12 (m, 3H), 6.95 (s, 1H), δ 5.32 (s, 1H), 4.49-4.14 (m, 2H), 3.75-3.33 (m, 8H), 3.11-2.81 (m, 4H), 2.71-2.48 (m, 4H), 2.26-2.11 (m, 1H), 2.00-1.82 (m, 1H), 1.51 (d, J=1.8 Hz, 3H), 1.30 (s, 9H); [M+H]⁺=802.5.

### Example 75: 3-(tert-butyl)-N-((1R)-1-(4-(7-(4-((1′-(2,6-dioxopiperidin-3-yl)-2′-oxospiro[cyclopropane-1,3′-indolin]-5′-yl)methyl)piperazin-1-yl)-6-methoxy-9H-pyrimido[4,5-b]indol-4-yl)-2-methylphenyl)ethyl)-1,2,4-oxadiazole-5-carboxamide

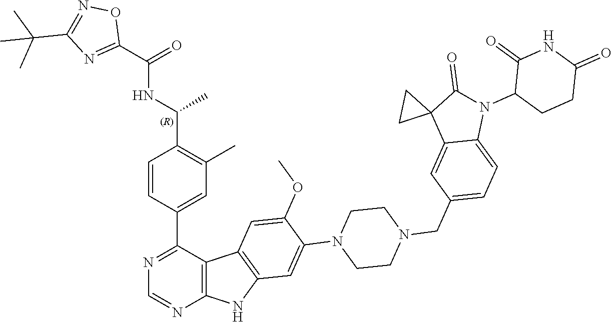

The titled compound was synthesized in the procedures similar to Example 47. ¹H NMR (500 MHz, DMSO) δ 12.15 (d, J=48.1 Hz, 1H), 11.03 (s, 1H), 9.88 (d, J=7.7 Hz, 1H), 8.79 (s, 1H), 7.75-7.68 (m, 2H), 7.65 (d, J=8.0 Hz, 1H), 7.46-7.30 (m, 1H), 7.21 (s, 1H), 7.15-7.01 (m, 1H), 7.00-6.87 (m, 2H), 5.40-5.23 (m, 2H), 4.26 (s, 1H), 3.64-3.48 (m, 4H), 3.46-3.38 (m, 1H), 3.36-3.29 (m, 1H), 3.18-3.07 (m, 2H), 3.06-2.94 (m, 2H), 2.91-2.78 (m, 1H), 2.67-2.51 (m, 2H), 2.46 (s, 3H), 1.97-1.85 (m, 1H), 1.67-1.54 (m, 4H), 1.51 (d, J=6.9 Hz, 3H), 1.30 (s, 9H), 1.17 (d, J=4.0 Hz, 2H); [M+H]⁺=851.5.

### Example 77: N-((1R)-1-(4-(7-(4-(4-((2,6-dioxopiperidin-3-yl)thio)benzyl)piperazin-1-yl)-6-methoxy-9H-pyrimido[4,5-b]indol-4-yl)-2-methylphenyl)ethyl)-3-(1-methylcyclopropyl)-1,2,4-oxadiazole-5-carboxamide

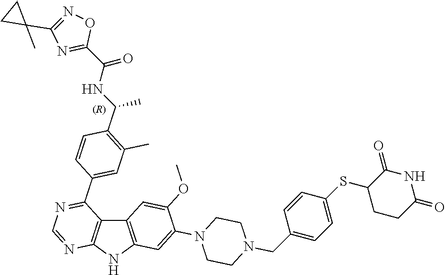

A stirred mixture of (R)—N-(1-(4-(6-methoxy-7-(piperazin-1-yl)-9H-pyrimido[4,5-b]indol-4-yl)-2-methylphenyl)ethyl)-3-(1-methylcyclopropyl)-1,2,4-oxadiazole-5-carboxamide 2,2,2-trifluoroacetic acid (the compound was obtained through the similar way in example 64) (0.1 g, 0.147 mmol), 3-((4-(hydroxymethyl)phenyl)thio)piperidine-2,6-dione (the compound was obtained through the same way in WO2019140387A1) (73.8 mg, 0.294 mmol) and (cyanomethyl)trimethylphosphonium iodide (107 mg, 441 mmol) in MeCN (10.0 mL) was stirred for 16 h at 100° C. under a nitrogen atmosphere. The resulting mixture was cooled and filtered. The residue was purified by prep-TLC (DCM/MeOH=7:1) to afford the product (10.0 mg, 8.5%). ¹H NMR (500 MHz, DMSO) δ 12.18 (d, J=34.6 Hz, 1H), 10.94 (d, J=30.2 Hz, 1H), 9.86 (d, J=7.6 Hz, 1H), 8.86 (d, J=17.3 Hz, 1H), 7.77 (d, J=12.1 Hz, 2H), 7.69 (d, J=7.9 Hz, 1H), 7.59 (s, 1H), 7.53 (d, J=8.1 Hz, 1H), 7.47 (d, J=8.0 Hz, 1H), 7.34 (d, J=7.6 Hz, 1H), 7.27 (d, J=14.6 Hz, 1H), 7.02 (d, J=17.9 Hz, 1H), 5.40-5.31 (m, 1H), 4.48-4.43 (m, 1H), 3.63-3.54 (m, 5H), 3.48-3.44 (m, 2H), 3.12-3.06 (m, 1H), 2.99-2.96 (m, 1H), 2.59-2.55 (m, 3H), 2.33-2.18 (m, 1H), 1.96-1.90 (m, 1H), 1.56 (d, J=6.9 Hz, 3H), 1.27-1.21 (m, 11H), 0.99 (s, 2H); [M+H]⁺=800.5.

### Example 78: 5-(tert-butyl)-N-((1R)-1-(4-(7-(4-((3-(2,6-dioxopiperidin-3-yl)-1-methyl-2-oxo-2,3-dihydro-1H-benzo[d]imidazol-5-yl)methyl)piperazin-1-yl)-9H-pyrimido[4,5-b]indol-4-yl)-3-fluoro-2-methylphenyl)ethyl)-1,2,4-oxadiazole-3-carboxamide

### Step 1: 4-bromo-N-methyl-2-nitroaniline

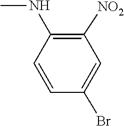

Into a 250 mL 3-necked round-bottom flask were added 4-bromo-1-fluoro-2-nitrobenzene (50.0 g, 0.227 mol), CH₃NH₂HCl (46.0 g, 0.681 mol) and DIEA (117.5 g, 0.908 mol) at room temperature. The resulting mixture was stirred for 6 h at 80° C. The mixture was allowed to cool down to room temperature and concentrated under reduced pressure. The residue was dissolved in water (500 mL). The resulting mixture was extracted with EtOAc (3×300 mL). The combined organic layers were washed with brine (2×200 mL) and dried over anhydrous Na₂SO₄. After filtration, the filtrate was concentrated under reduced pressure to afford the product (60.0 g, crude). [M+H]⁺=231.0.

### Step 2: 4-bromo-N1-methylbenzene-1,2-diamine

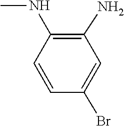

To a mixture of 4-bromo-N-methyl-2-nitroaniline (65.7 g, 0.285 mol) in AcOH (328 mL), H₂O (160 mL) and EtOAc (328 mL) was added Fe (64.0 g, 1.14 mol) at ambient temperature. The resulting mixture was stirred for 4 h at 80° C. The mixture was allowed to cool down to room temperature. The resulting mixture was filtered and the filtrate was concentrated under reduced pressure. Then the residue was diluted with H₂O (100 mL) and extracted with EtOAc (3×300 mL). The combined organic layers were washed with aq. NaHCO₃ (3×200 mL) and brine (300 mL) and dried over anhydrous Na₂SO₄. After filtration, the filtrate was concentrated under reduced pressure. The residue was purified by silica gel column chromatography, eluted with PE/EtOAc (5:1) to afford the product (35.6 g, 59%). [M+H]⁺=200.1.

### Step 3: 5-bromo-1-methyl-1,3-dihydro-2H-benzo[d]imidazol-2-one

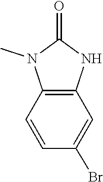

To a stirred solution of 4-bromo-N1-methylbenzene-1,2-diamine (33.6 g, 167.108 mmol) in ACN (404 mL) was added CDI (54.2 g, 0.334 mmol) in portions at room temperature under nitrogen atmosphere. The resulting mixture was stirred overnight at 80° C. and concentrated under reduced pressure. The resulting mixture was diluted with water (300 mL). The resulting mixture was filtered and the filter cake was washed with water (3×200 mL) and dried under reduced pressure to afford the product (30 g, 79%). [M+H]⁺=227.1.

### Step 4: 3-(6-bromo-3-methyl-2-oxo-2,3-dihydro-1H-benzo[d]imidazol-1-yl)-1-(4-methoxybenzyl)piperidine-2,6-dione

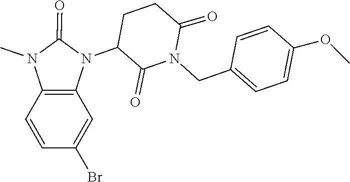

To a stirred solution of 5-bromo-1-methyl-1,3-dihydro-2H-benzo[d]imidazol-2-one (5.0 g, 22.021 mmol) in THF (100 mL) was added t-BuOK (3.0 g, 26.735 mmol) in portions at 0° C. under nitrogen atmosphere. The resulting mixture was stirred for 30 min at 0° C. To the above mixture was added 1-[(4-methoxyphenyl)methyl]-2,6-dioxopiperidin-3-yl trifluoromethanesulfonate (8.5 g, 22.291 mmol) in portions at 0° C. The resulting mixture was stirred for additional 1 h at room temperature. The reaction was quenched with sat. NH₄Cl (aq.) (40 mL) at 0° C. and diluted with water (500 mL). The resulting mixture was extracted with EtOAc (3×200 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EtOAc (1:1) to afford the product (6.8 g, 67%). [M+H]⁺=458.0.

### Step 5: 3-(6-bromo-3-methyl-2-oxo-2,3-dihydro-1H-benzo[d]imidazol-1-yl)piperidine-2,6-dione

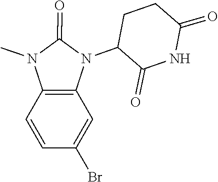 [SMILES: Cn1c(=O)n(C2CCC(=O)NC2=O)c2cc(Br)ccc21]

To a stirred solution of 3-(6-bromo-3-methyl-2-oxo-2,3-dihydro-1H-benzo[d]imidazol-1-yl)-1-(4-methoxybenzyl)piperidine-2,6-dione (6.2 g, 13.593 mmol) in toluene (40 mL) was added CH₃SO₃H (20 mL, 208.104 mmol) in portions at 15° C. under nitrogen atmosphere. The resulting mixture was stirred for 3 h at 120° C. The mixture was allowed to cool down to room temperature and concentrated under reduced pressure. The residue was poured into ice/water (30 mL) and extracted with EtOAc (3×20 mL). The combined organic layer was washed with brine (10 mL) and dried over anhydrous Na₂SO₄. After filtration, the filtrate was concentrated under reduced pressure. The residue was triturated with EtOAc (10 mL) and filtered. The filtrate cake was collected and dried under reduced pressure to afford the product (2.7 g, 53%). [M+H]⁺=338.1.

### Step 6: 3-(2,6-dioxopiperidin-3-yl)-1-methyl-2-oxo-2,3-dihydro-1H-benzo[d]imidazole-5-carbaldehyde

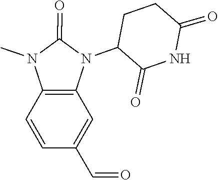 [SMILES: Cn1c(=O)n(C2CCC(=O)NC2=O)c2cc(C=O)ccc21]

To a stirred solution of 3-(6-bromo-3-methyl-2-oxo-2,3-dihydro-1H-benzo[d]imidazol-1-yl)piperidine-2,6-dione (1.0 g, 2.957 mmol), TEA (897 mg, 8.871 mmol) and Et₃SiH (687 mg, 5.914 mmol) in DMA (20 mL, 0.230 mmol) was added Pd(dppf)Cl₂ (325 mg, 0.444 mmol) in portions at room temperature under nitrogen atmosphere. The resulting mixture was stirred for 16 h at 90° C. under CO atmosphere. The mixture was allowed to cool down to room temperature. The residue was dissolved in water (20 mL). The resulting mixture was extracted with EtOAc (2×30 mL). The combined organic layers were washed with brine (3×30 mL) and dried over anhydrous Na₂SO₄. After filtration, the filtrate was concentrated under reduced pressure. The residue product was purified by reverse phase flash chromatography with the following conditions (Column: Spherical C18, 20-40 μm, 330 g; Mobile Phase A: Water (plus 5 mM NH₄HCO₃); Mobile Phase B: ACN; Flow rate: 80 mL/min; Gradient: 5%-5% B, 10 min, 55% B-80% B gradient in 30 min; Detector: 220 nm. The fractions containing the desired product were collected at 75% B to afford the product (266 mg, 28%). ¹H NMR (400 MHz, DMSO) δ 9.92 (s, 1H), 8.52 (s, 1H), 7.80-7.58 (m, 2H), 7.42 (d, J=8.1 Hz, 1H), 7.21-6.99 (m, 1H), 5.51 (dd, J=12.8, 5.3 Hz, 1H), 3.35 (s, 1H), 2.99-2.84 (m, 1H), 2.81-2.58 (m, 2H), 2.16-1.93 (m, 2H); [M+H]+=288.1.

### Step 7: 5-(tert-butyl)-N-((1R)-1-(4-(7-(4-((3-(2,6-dioxopiperidin-3-yl)-1-methyl-2-oxo-2,3-dihydro-1H-benzo[d]imidazol-5-yl)methyl)piperazin-1-yl)-9H-pyrimido[4,5-b]indol-4-yl)-3-fluoro-2-methylphenyl)ethyl)-1,2,4-oxadiazole-3-carboxamide

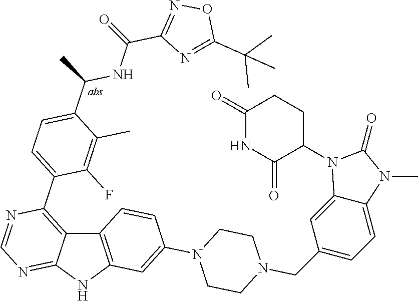 [SMILES: Cc1c([C@@H](C)NC(=O)c2noc(C(C)(C)C)n2)ccc(-c2ncnc3[nH]c4cc(N5CCN(Cc6ccc7c(c6)n(C6CCC(=O)NC6=O)c(=O)n7C)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 3-(2,6-dioxopiperidin-3-yl)-1-methyl-2-oxo-2,3-dihydro-1H-benzo[d]imidazole-5-carbaldehyde (66 mg, 0.230 mmol) and HOAc (0.06 mL) were added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%:0%˜90%:10% gradient elution) to give crude product, which was further purified by prep-HPLC to afford the desired product (23.14 mg, 15%). ¹H NMR (500 MHz, DMSO) δ 12.17 (s, 1H), 11.09 (s, 1H), 9.59 (d, J=10.0 Hz, 1H), 8.84 (s, 1H), 7.56-7.47 (m, 2H), 7.23-7.18 (m, 1H), 7.16-7.10 (m, 2H), 7.07 (d, J=10.0 Hz, 1H), 6.91 (d, J=7.2 Hz, 2H), 5.47-5.34 (m, 2H), 3.53 (s, 1H), 3.33 (s, 2H), 3.29 (s, 1H), 3.24 (s, 4H), 2.95-2.83 (m, 1H), 2.77-2.66 (m, 1H), 2.62 (d, J=15.0 Hz, 1H), 2.53 (s, 5H), 2.39 (s, 3H), 2.05-1.99 (m, 1H), 1.56 (d, J=10.0 Hz, 3H), 1.43 (s, 9H); [M+H]⁺=828.5.

### Example 79: 5-(tert-butyl)-N-((1R)-1-(4-(7-(4-((1′-(2,6-dioxopiperidin-3-yl)-2′-oxospiro[cyclopropane-1,3′-indolin]-5′-yl)methyl)piperazin-1-yl)-9H-pyrimido[4,5-b]indol-4-yl)-3-fluoro-2-methylphenyl)ethyl)-1,2,4-oxadiazole-3-carboxamide

### Step 1: methyl 2-(5-bromo-2-nitrophenyl)acrylate

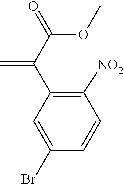 [SMILES: C=C(C(=O)OC)c1cc(Br)ccc1[N+](=O)[O-]]

To a stirred solution of N,N,N-triethylanilinium iodide (13.36 g, 43.784 mmol) and methyl 2-(5-bromo-2-nitrophenyl)acetate (10 g, 36.487 mmol) in toluene (200 mL) were added (CH₂O)ₙ (7.12 g, 237.166 mmol) and K₂CO₃ (10.09 g, 72.974 mmol) at room temperature. The resulting mixture was heated to 80° C. over 40 min. The resulting mixture was stirred for 1 h at 80° C. and concentrated under reduced pressure. The residue was purified by silica gel column chromatography, eluted with EtOAc in petroleum ether (0%-15%) to afford the product (8.7 g, 83.35%). [M−H]⁻=284.0.

### Step 2: methyl 1-(5-bromo-2-nitrophenyl)cyclopropane-1-carboxylate

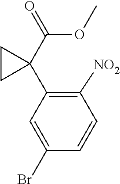 [SMILES: COC(=O)C1(c2cc(Br)ccc2[N+](=O)[O-])CC1]

A mixture of t-BuOK (3.77 g, 33.557 mmol) and trimethyloxosulfonium iodide (7.38 g, 33.557 mmol) in THF (160 mL) was stirred for 0.5 h at 0° C. under nitrogen atmosphere. To the above mixture was added methyl 2-(5-bromo-2-nitrophenyl)acrylate (8 g, 27.964 mmol) in THF (20 mL) dropwise at 0° C. The resulting mixture was stirred for 2 h at room temperature. The reaction was quenched with sat. NH₄Cl (aq.) (20 mL) at 0° C. and diluted with water (1 L). The resulting mixture was extracted with EtOAc (3×200 mL). The combined organic layers were washed with brine (2×100 mL) and dried over anhydrous Na₂SO₄. After filtration, the filtrate was concentrated under reduced pressure. The residue was purified by silica gel column chromatography, eluted with EtOAc in petroleum ether (0%-15%) to afford the product (6.6 g, 78.64%). [M-CH₂+H]=285.9.

### Step 3: 5′-bromospiro[cyclopropane-1,3′-indolin]-2′-one

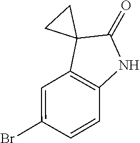 [SMILES: O=C1Nc2ccc(Br)cc2C12CC2]

To a stirred solution of methyl 1-(5-bromo-2-nitrophenyl)cyclopropane-1-carboxylate (6.6 g, 21.992 mmol) in AcOH (66 mL) was added Fe (12.28 g, 219.921 mmol) in portions at room temperature. The resulting mixture was stirred for 2 h at room temperature and concentrated under reduced pressure. The residue was purified by silica gel column chromatography, eluted with EtOAc in petroleum ether (20%-90%) to afford the product (5 g, 95.49%). [M+H]⁺=237.9.

### Step 4: 3-(5′-bromo-2′-oxospiro[cyclopropane-1,3′-indolin]-1′-yl)-1-(4-methoxybenzyl)piperidine-2,6-dione

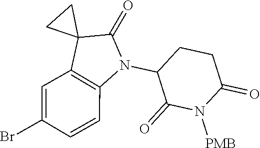

To a stirred mixture of 5′-bromospiro[cyclopropane-1,3′-indolin]-2′-one (5 g, 21.001 mmol) in THF (40 mL) was added t-BuOK (2.83 g, 25.201 mmol) in portions at 0° C. under nitrogen atmosphere. The resulting mixture was stirred for 1 h at room temperature under nitrogen atmosphere. To the above mixture was added 1-[(4-methoxyphenyl)methyl]-2,6-dioxopiperidin-3-yl trifluoromethanesulfonate (9.61 g, 25.201 mmol) in THF (20 mL) at 0° C. The resulting mixture was stirred for 2 h at room temperature. The reaction was quenched with sat. NH₄Cl (aq.) (100 mL) at 0° C. and extracted with EtOAc (3×300 mL). The combined organic layers were washed with brine (2×200 mL) and dried over anhydrous Na₂SO₄. After filtration, the filtrate was concentrated under reduced pressure. The residue was purified by silica gel column chromatography, eluted with EtOAc in petroleum ether (10%-50%) to afford the product (7 g, 71.02%). ¹H NMR (400 MHz, DMSO) δ 7.36-7.28 (m, 2H), 7.25-7.12 (m, 2H), 6.91-6.79 (m, 3H), 5.53-5.45 (m, 1H), 4.87-4.72 (m, 2H), 3.73 (s, 3H), 3.12-2.98 (m, 1H), 2.85-2.75 (m, 1H), 2.74-2.58 (m, 1H), 2.07-1.97 (m, 1H), 1.82-1.65 (m, 2H), 1.67-1.56 (m, 2H); [M+H]⁺=469.0.

### Step 5: 3-(5′-bromo-2′-oxospiro[cyclopropane-1,3′-indolin]-1′-yl)piperidine-2,6-dione

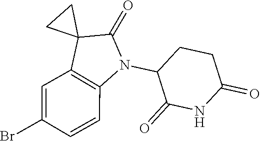 [SMILES: O=C1CCC(N2C(=O)C3(CC3)c3cc(Br)ccc32)C(=O)N1]

A solution of 3-(5′-bromo-2′-oxospiro[cyclopropane-1,3′-indolin]-1′-yl)-1-(4-methoxybenzyl)piperidine-2,6-dione (2 g, 4.261 mmol) and (NH₄)₂Ce(NO₃)₆ (9.38 g, 17.045 mmol) in MeCN (50 mL) and H₂O (10 mL) was stirred for 4 h at room temperature. The reaction was quenched by the addition of sat. NaHSO₃ (aq.) (10 mL) at room temperature. The resulting mixture was diluted with water (80 mL) and extracted with EtOAc (3×100 mL). The combined organic layers were washed with brine (2×50 mL) and dried over anhydrous Na₂SO₄. After filtration, the filtrate was concentrated under reduced pressure. The residue was purified by silica gel column chromatography, eluted with EtOAc in petroleum ether (10%-60%) to afford the product (950 mg, 63.84%). [M+H]⁺=349.0.

### Step 6: 1′-(2,6-dioxopiperidin-3-yl)-2′-oxospiro[cyclopropane-1,3′-indoline]-5′-carbaldehyde

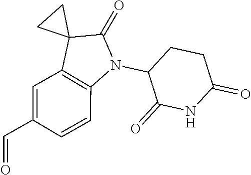 [SMILES: O=Cc1ccc2c(c1)C1(CC1)C(=O)N2C1CCC(=O)NC1=O]

To a stirred solution of 3-(5′-bromo-2′-oxospiro[cyclopropane-1,3′-indolin]-1′-yl)piperidine-2,6-dione (900 mg, 2.577 mmol) and Et₃SiH (1.25 mL, 10.739 mmol) in DMA (36 mL) were added TEA (1.07 mL, 10.621 mmol) and Pd(dppf)Cl₂ (283 mg, 0.387 mmol) at 90° C. under nitrogen atmosphere. The resulting mixture was stirred for 16 h at 90° C. under CO atmosphere. The resulting mixture was purified by reverse phase flash chromatography with the following conditions: column, C18; mobile phase, MeCN in water (0.1% FA), 20% to 50% gradient in 15 min; detector, UV 254 nm to afford the product (311.9 mg, 36.51%). ¹H NMR (400 MHz, DMSO) δ 11.15 (s, 1H), 9.88 (s, 1H), 7.84 (dd, J=8.0, 1.6 Hz, 1H), 7.62 (d, J=1.6 Hz, 1H), 7.27 (d, J=7.6 Hz, 1H), 5.44 (d, J=7.2 Hz, 1H), 2.91 (s, 1H), 2.76-2.59 (m, 2H), 2.09-1.98 (m, 1H), 1.89-1.78 (m, 2H), 1.72-1.59 (m, 2H); [M+H]⁺=299.3.

### Step 7: 5-(tert-butyl)-N-((1R)-1-(4-(7-(4-((1′-(2,6-dioxopiperidin-3-yl)-2′-oxospiro[cyclopropane-1,3′-indolin]-5′-yl)methyl)piperazin-1-yl)-9H-pyrimido[4,5-b]indol-4-yl)-3-fluoro-2-methylphenyl)ethyl)-1,2,4-oxadiazole-3-carboxamide

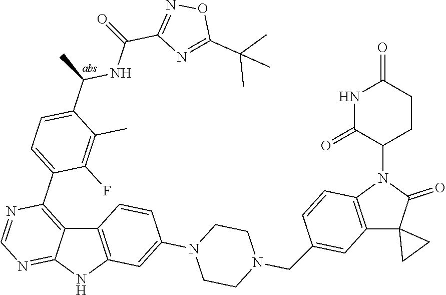 [SMILES: Cc1c([C@@H](C)NC(=O)c2noc(C(C)(C)C)n2)ccc(-c2ncnc3[nH]c4cc(N5CCN(Cc6ccc7c(c6)C6(CC6)C(=O)N7C6CCC(=O)NC6=O)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 1′-(2,6-dioxopiperidin-3-yl)-2′-oxospiro[cyclopropane-1,3′-indoline]-5′-carbaldehyde (53 mg, 0.177 mmol) and HOAc (0.06 mL) were added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-HPLC to afford the desired product (12.44 mg, 8%). ¹H NMR (500 MHz, DMSO) δ 12.18 (s, 1H), 11.08 (s, 1H), 9.59 (d, J=5.0 Hz, 1H), 8.84 (s, 1H), 7.59-7.49 (m, 2H), 7.24-7.14 (m, 2H), 7.05-6.94 (m, 2H), 6.94-6.84 (m, 2H), 5.47-5.41 (m, 1H), 5.33 (s, 1H), 3.47 (s, 2H), 3.29-3.27 (m, 1H), 3.23 (s, 4H), 2.93-2.84 (m, 1H), 2.68-2.58 (m, 2H), 2.51 (s, 2H), 2.47-2.45 (m, 1H), 2.39 (s, 3H), 2.00-1.93 (m, 1H), 1.66 (d, J=3.5 Hz, 2H), 1.55 (d, J=10.0 Hz, 4H), 1.43 (s, 9H); [M+H]⁺=839.5.

### Example 80: 5-(tert-butyl)-N-((1R)-1-(4-(7-(4-(4-((2,6-dioxopiperidin-3-yl)thio)phenethyl)piperazin-1-yl)-9H-pyrimido[4,5-b]indol-4-yl)-3-fluoro-2-methylphenyl)ethyl)-1,2,4-oxadiazole-3-carboxamide

### Step 1: 2-(4-mercaptophenyl)ethan-1-ol

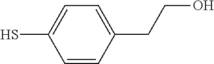

To a stirred solution of 4-mercaptophenylacetic acid (2.5 g, 14.9 mmol) in THF (30 mL) was added B₂H₆-THF (37.3 mL, 37.3 mmol, 1 M in THF) dropwise at 0° C. under nitrogen atmosphere. The resulting mixture was stirred for 2 h at room temperature under nitrogen atmosphere. The reaction was quenched by the addition of sat. NH₄Cl (aq.) (10 mL) and HCl (1 M) (10 mL) at 0° C. The resulting mixture was extracted with EtOAc (3×20 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EtOAc (2:1 to 1:1) to afford the product (1.8 g, 78%). Step 2: 3-((4-(2-hydroxyethyl)phenyl)thio)piperidine-2,6-dione

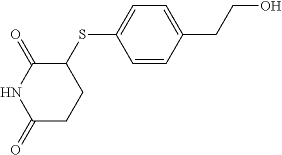 [SMILES: O=C1CCC(Sc2ccc(CCO)cc2)C(=O)N1]

To a stirred solution of 2-(4-mercaptophenyl)ethan-1-ol (1.8 g, 11.7 mmol) and Cs₂CO₃ (5.7 g, 17.5 mmol) in DMF (36 mL) was added 3-bromopiperidine-2,6-dione (2.2 g, 11.7 mmol) in portions at room temperature. The resulting mixture was stirred for 1 h at room temperature under nitrogen atmosphere and diluted with water (100 mL). The resulting mixture was extracted with EtOAc (3×40 mL). The combined organic layers were washed with brine (2×40 mL) and dried over anhydrous Na₂SO₄. After filtration, the filtrate was concentrated under reduced pressure. The residue was purified by silica gel column chromatography, eluted with CH₂Cl₂/EtOAc (2:1 to 1:1) to afford the product (1.1 g, 36%). ¹H NMR (300 MHz, DMSO) δ 10.88 (s, 1H), 7.47-7.35 (m, 2H), 7.26-7.17 (m, 2H), 4.64 (t, J=5.2 Hz, 1H), 4.23 (dd, J=7.8, 4.7 Hz, 1H), 3.60 (td, J=6.9, 5.2 Hz, 2H), 2.71 (t, J=6.9 Hz, 2H), 2.56 (d, J=6.5 Hz, 2H), 2.21-2.17 (m, 1H), 1.99-1.82 (m, 1H); [M−H]⁻=264.0.

### Step 3: 5-(tert-butyl)-N-((1R)-1-(4-(7-(4-(4-((2,6-dioxopiperidin-3-yl)thio)phenethyl)piperazin-1-yl)-9H-pyrimido[4,5-b]indol-4-yl)-3-fluoro-2-methylphenyl)ethyl)-1,2,4-oxadiazole-3-carboxamide

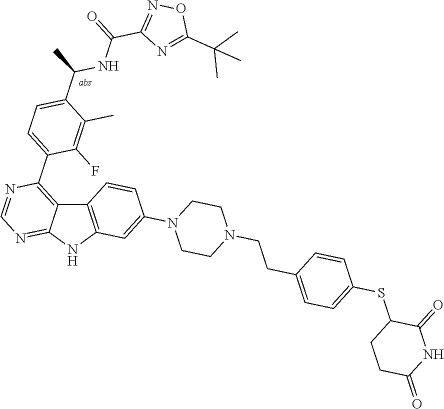 [SMILES: Cc1c([C@@H](C)NC(=O)c2noc(C(C)(C)C)n2)ccc(-c2ncnc3[nH]c4cc(N5CCN(CCc6ccc(SC7CCC(=O)NC7=O)cc6)CC5)ccc4c23)c1F]

A mixture of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (80 mg, 0.144 mmol), 3-((4-(2-hydroxyethyl)phenyl)thio)piperidine-2,6-dione (76 mg, 0.288 mmol), 2-(iodotrimethyl-15-phosphaneyl)acetonitrile (70 mg, 0.288 mmol) and DIEA (37 mg, 0.288 mmol) in ACN (10 mL) was stirred in a sealed vial at 100° C. overnight. After being cooled to room temperature, the mixture was evaporated in vacuum, and purified with silica gel column chromatography (DCM:MeOH=100:0˜90:10 gradient elution) to give the crude product, which was further purified by prep-HPLC to give the product (9.5 mg, 8%). ¹H NMR (500 MHz, DMSO) δ 12.26 (d, J=70.0 Hz, 1H), 10.89 (s, 1H), 9.82-9.56 (m, 1H), 8.87 (d, J=18.4 Hz, 1H), 7.58-7.51 (m, 2H), 7.45 (d, J=30.0 Hz, 2H), 7.27 (s, 3H), 7.07-6.86 (m, 2H), 5.48-5.40 (m, 1H), 4.33-4.21 (m, 1H), 3.98 (s, 1H), 3.68 (s, 1H), 3.43 (s, 1H), 3.24 (s, 3H), 3.16-3.11 (m, 1H), 3.04 (s, 1H), 2.78 (s, 1H), 2.61 (s, 1H), 2.57-2.53 (m, 2H), 2.40 (s, 3H), 2.25-2.16 (m, 1H), 1.96-1.87 (m, 1H), 1.56 (d, J=10.0 Hz, 3H), 1.43 (s, 9H); [M+H]⁺=804.4.

### Example 81: 1-(4-(4-((4-(4-(4-((3-methyl-2-oxoimidazolidin-1-yl)methyl)piperidin-1-yl)-9H-pyrimido[4,5-b]indol-7-yl)piperazin-1-yl)methyl)piperidin-1-yl)phenyl)dihydropyrimidine-2,4(1H,3H)-dione

### Step 1: tert-butyl 4-((3-methyl-2-oxoimidazolidin-1-yl)methyl)piperidine-1-carboxylate

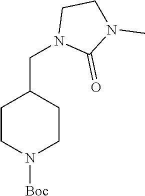 [SMILES: CN1CCN(CC2CCN(C(=O)OC(C)(C)C)CC2)C1=O]

To a solution of 1-methylimidazolidin-2-one (1 g, 10 mmol) in DMF (30 mL), NaH (0.6 g, 15 mmol, 60%) was added in portions at 0° C.˜5° C. The mixture was stirred at 0° C. for 30 mins. Then tert-butyl 4-(bromomethyl)piperidine-1-carboxylate (2.78 g, 10 mmol) was added. The mixture was stirred at room temperature overnight. The mixture was extracted with EA (30 mL *3), washed with brine (50 mL), dried over Na₂SO₄, and concentrated in vacuo to give the titled product (2.3 g, 77%). [M-56]⁺=242.1.

### Step 2: 1-methyl-3-(piperidin-4-ylmethyl)imidazolidin-2-one hydrochloride

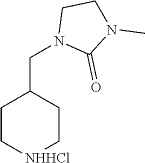

A mixture of tert-butyl 4-((3-methyl-2-oxoimidazolidin-1-yl)methyl)piperidine-1-carboxylate (500 mg, 1.68 mmol) in 4 M HCl in dioxane (10 mL) was stirred at room temperature for 2.5 hours. Then the mixture was concentrated in vacuo to give the product (450 mg, crude), which was used in next step without further purification. [M+H]⁺=198.2.

### Step 3: benzyl 4-(4-(4-((3-methyl-2-oxoimidazolidin-1-yl)methyl)piperidin-1-yl)-9H-pyrimido[4,5-b]indol-7-yl)piperazine-1-carboxylate

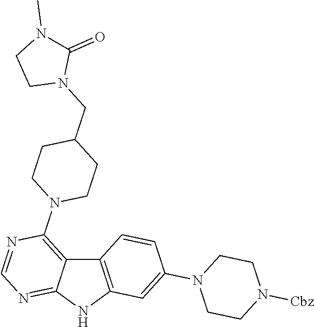 [SMILES: CN1CCN(CC2CCN(c3ncnc4[nH]c5cc(N6CCN(C(=O)OCc7ccccc7)CC6)ccc5c34)CC2)C1=O]

A mixture of benzyl 4-(4-chloro-9H-pyrimido[4,5-b]indol-7-yl)piperazine-1-carboxylate (100 mg, 0.238 mmol), 1-methyl-3-(piperidin-4-ylmethyl)imidazolidin-2-one hydrochloride (220 mg, 1.07 mmol) and DIEA (307 mg, 2.38 mmol) in i-PrOH (10 mL) was stirred at 93° C. overnight. Then the mixture was allowed to cool and concentrated in vacuo. The residue was purified by silica gel column chromatography (PE:EA=100%: 0%˜80%: 20%) to give the product (100 mg, 72%). [M+H]⁺=583.6.

### Step 4: 1-methyl-3-((1-(7-(piperazin-1-yl)-9H-pyrimido[4,5-b]indol-4-yl)piperidin-4-yl)methyl)imidazolidin-2-one

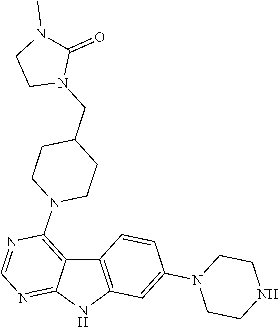 [SMILES: CN1CCN(CC2CCN(c3ncnc4[nH]c5cc(N6CCNCC6)ccc5c34)CC2)C1=O]

A mixture of benzyl 4-(4-(4-((3-methyl-2-oxoimidazolidin-1-yl)methyl)piperidin-1-yl)-9H-pyrimido[4,5-b]indol-7-yl)piperazine-1-carboxylate (100 mg, 0.172 mmol) in TFA (10 mL) was stirred under reflux for 3 hours. Then the mixture was concentrated in vacuo to give the product (80 mg, crude), which was used in next step without further purification. [M+H]⁺=449.4.

### Step 5: 1-(4-(4-((4-(4-(4-((3-methyl-2-oxoimidazolidin-1-yl)methyl)piperidin-1-yl)-9H-pyrimido[4,5-b]indol-7-yl)piperazin-1-yl)methyl)piperidin-1-yl)phenyl)dihydropyrimidine-2,4(1H,3H)-dione

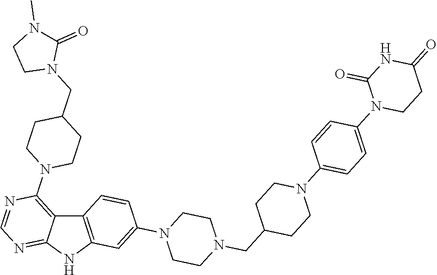 [SMILES: CN1CCN(CC2CCN(c3ncnc4[nH]c5cc(N6CCN(CC7CCN(c8ccc(N9CCC(=O)NC9=O)cc8)CC7)CC6)ccc5c34)CC2)C1=O]

To a solution of 1-methyl-3-((1-(7-(piperazin-1-yl)-9H-pyrimido[4,5-b]indol-4-yl)piperidin-4-yl)methyl)imidazolidin-2-one (80 mg, crude) in dichloromethane (4 mL) and MeOH (4 mL), 1-(4-(2,4-dioxotetrahydropyrimidin-1(2H)-yl)phenyl)piperidine-4-carbaldehyde (70 mg, 0.232 mmol) and HOAc (0.06 mL) were added. The mixture was stirred at room temperature overnight. Then NaBH(OAc)₃ (113 mg, 0.537 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product (15.29 mg, 11%). ¹H NMR (500 MHz, DMSO) δ 12.00-11.75 (m, 1H), 10.26 (s, 1H), 8.37 (s, 1H), 7.54 (s, 1H), 7.14 (d, J=10.0 Hz, 2H), 7.04 (s, 1H), 6.94 (d, J=6.9 Hz, 3H), 4.20 (d, J=10.0 Hz, 2H), 3.77-3.62 (m, 5H), 3.28-3.07 (m, 10H), 3.04 (d, J=10.0 Hz, 2H), 2.98 (t, J=10.0 Hz, 2H), 2.73-2.67 (m, 4H), 2.66 (d, J=5.0 Hz, 3H), 2.55 (s, 2H), 2.24 (s, 1H), 1.85 (s, 2H), 1.79 (d, J=10.0 Hz, 2H), 1.75-1.64 (m, 1H), 1.35 (dd, J=20.0, 10.0 Hz, 3H), 1.24 (s, 2H); [M+H]⁺=734.5.

### Example 82: 5-(tert-butyl)-N-((1R)-1-(4-(7-(4-(2-(1-(2,6-dioxopiperidin-3-yl)-3-methyl-2-oxo-2,3-dihydro-1H-benzo[d]imidazol-4-yl)ethyl)piperazin-1-yl)-9H-pyrimido[4,5-b]indol-4-yl)-3-fluoro-2-methylphenyl)ethyl)-1,2,4-oxadiazole-3-carboxamide

### Step 1: 3-(4-bromo-3-methyl-2-oxo-2,3-dihydro-1H-benzo[d]imidazol-1-yl)piperidine-2,6-dione

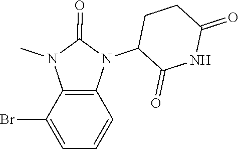 [SMILES: Cn1c(=O)n(C2CCC(=O)NC2=O)c2cccc(Br)c21]

A mixture of 3-(4-bromo-3-methyl-2-oxo-1,3-benzodiazol-1-yl)-1-[(4-methoxyphenyl)methyl]piperidine-2,6-dione (5.9 g, 12.873 mmol) and CH₃SO₃H (17 mL, 257.482 mmol) in toluene (30 mL) was stirred for 2 h at 120° C. under nitrogen atmosphere. The mixture was allowed to cool down to room temperature. The resulting mixture was concentrated under reduced pressure. The reaction was quenched with water/ice at 0° C. The resulting mixture was added EtOAc (50 mL) and filtered and the filter cake was washed with EtOAc (3×10 mL). The filtrate was extracted with EtOAc (3×50 mL). The combined organic layers were washed with brine (100 mL) and dried over anhydrous Na₂SO₄. After filtration, the filtrate was concentrated under reduced pressure. To the resulting mixture was added EtOAc (10 mL) and filtered and the filter cake was washed with EtOAc (3×5 mL). This resulted in the product (3 g, 69%). [M+H]⁺=338.0.

### Step 2: 3-(4-allyl-3-methyl-2-oxo-2,3-dihydro-1H-benzo[d]imidazol-1-yl)piperidine-2,6-dione

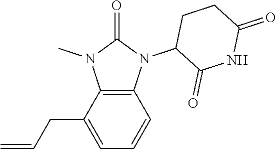 [SMILES: C=CCc1cccc2c1n(C)c(=O)n2C1CCC(=O)NC1=O]

To a stirred mixture of 3-(4-bromo-3-methyl-2-oxo-2,3-dihydro-1H-benzo[d]imidazol-1-yl)piperidine-2,6-dione (2.0 g, 5.914 mmol) and 4,4,5,5-tetramethyl-2-(prop-2-en-1-yl)-1,3,2-dioxaborolane (2.0 g, 11.842 mmol) in DMA (40 mL) were added DBU (1.8 g, 11.824 mmol) and Pd(dppf)Cl₂ (649 mg, 0.887 mmol) at room temperature under nitrogen atmosphere. The resulting mixture was stirred overnight at 90° C. under nitrogen atmosphere. The mixture was allowed to cool down to room temperature. To the resulting mixture was added water (100 mL) and extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with PE/EtOAc (10:1 to 1:1) to afford the product (1.1 g, 57%). [M+H]⁺=300.0.

### Step 3: 2-(1-(2,6-dioxopiperidin-3-yl)-3-methyl-2-oxo-2,3-dihydro-1H-benzo[d]imidazol-4-yl)acetaldehyde

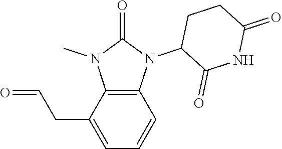 [SMILES: Cn1c(=O)n(C2CCC(=O)NC2=O)c2cccc(CC=O)c21]

To a stirred mixture of 3-(4-allyl-3-methyl-2-oxo-2,3-dihydro-1H-benzo[d]imidazol-1-yl)piperidine-2,6-dione (1.0 g, 3.341 mmol) and NaIO₄ (2.8 g, 13.371 mmol) in dioxane (20 mL) and H₂O (5 mL) was added K₂OsO₄·2H₂O (24 mg, 0.067 mmol) at 0° C. The resulting mixture was stirred for 2 h at room temperature. The reaction was quenched by the addition of sat. Na₂S₂O₃ (aq.) at 0° C. The resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by silica gel column chromatography, eluted with PE/EtOAc (10:1 to 1:1) to afford the product (150 mg, 15%). ¹H NMR (400 MHz, DMSO) δ 11.10 (s, 1H), 9.81 (s, 1H), 7.11-6.97 (m, 2H), 6.88 (d, J=7.6 Hz, 1H), 5.39 (dd, J=12.6, 5.4 Hz, 1H), 4.21 (s, 2H), 3.43 (s, 3H), 2.75-2.87 (m, 1H), 2.79-2.56 (m, 3H); [M+H]⁺=302.1.

### Step 4: 5-(tert-butyl)-N-((1R)-1-(4-(7-(4-(2-(1-(2,6-dioxopiperidin-3-yl)-3-methyl-2-oxo-2,3-dihydro-1H-benzo[d]imidazol-4-yl)ethyl)piperazin-1-yl)-9H-pyrimido[4,5-b]indol-4-yl)-3-fluoro-2-methylphenyl)ethyl)-1,2,4-oxadiazole-3-carboxamide

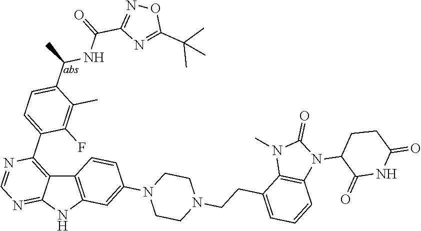

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 2-(1-(2,6-dioxopiperidin-3-yl)-3-methyl-2-oxo-2,3-dihydro-1H-benzo[d]imidazol-4-yl)acetaldehyde (53 mg, 0.177 mmol) and HOAc (0.06 mL) was added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%: 0%˜90%: 10% gradient elution) to give the crude product, which was further purified by prep-HPLC to afford the desired product (14.28 mg, 9%). ¹H NMR (500 MHz, DMSO) δ 12.19 (s, 1H), 11.09 (s, 1H), 9.60 (d, J=5.0 Hz, 1H), 8.85 (s, 1H), 7.64-7.41 (m, 2H), 7.22 (d, J=10.0 Hz, 1H), 7.01-6.92 (m, 5H), 5.51-5.31 (m, 2H), 3.61 (s, 3H), 3.30-3.24 (m, 3H), 3.12 (s, 2H), 2.95-2.85 (m, 1H), 2.76-2.71 (m, 1H), 2.69-2.59 (m, 5H), 2.40 (s, 3H), 2.04-1.96 (m, 1H), 1.57 (d, J=5.0 Hz, 3H), 1.44 (s, 9H); [M+H]⁺=842.5.

### Example 83: 3-(tert-butyl)-N-((1R)-1-(4-(7-(4-((1-(2,6-dioxopiperidin-3-yl)-3,3-dimethyl-2-oxoindolin-5-yl)methyl)piperazin-1-yl)-6-methoxy-9H-pyrimido[4,5-b]indol-4-yl)-5-fluoro-2-methylphenyl)ethyl)-1,2,4-oxadiazole-5-carboxamide

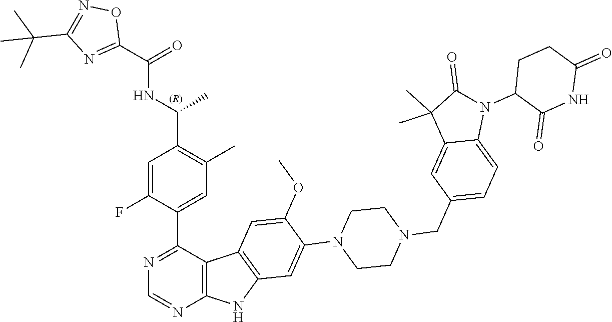

The titled compound was synthesized in the procedures similar to Example 47. ¹H NMR (500 MHz, DMSO) δ 12.17 (s, 1H), 11.07 (s, 1H), 9.54 (d, J=7.8 Hz, 1H), 8.88 (s, 1H), 7.58-7.48 (m, 2H), 7.33 (d, J=1.1 Hz, 1H), 7.18 (d, J=7.9 Hz, 1H), 7.00 (s, 1H), 6.94 (d, J=7.6 Hz, 1H), 6.77 (d, J=1.5 Hz, 1H), 5.40-5.32 (m, 1H), 5.27-5.18 (m, 1H), 3.54 (s, 3H), 3.50 (s, 2H), 3.14-2.99 (m, 4H), 2.89 (dd, J=21.5, 10.6 Hz, 1H), 2.69-2.53 (m, 6H), 2.47 (s, 3H), 2.00-1.92 (m, 1H), 1.54 (d, J=7.0 Hz, 3H), 1.44 (s, 9H), 1.30 (d, J=2.8 Hz, 6H); [M+H]⁺=871.7.

### Example 84: 5-(tert-butyl)-N-((1R)-1-(4-(7-(4-((1-(2,6-dioxopiperidin-3-yl)-3,3-dimethyl-2-oxoindolin-4-yl)methyl)piperazin-1-yl)-9H-pyrimido[4,5-b]indol-4-yl)-3-fluoro-2-methylphenyl)ethyl)-1, 2,4-oxadiazole-3-carboxamide

### Step 1: 3-(4-bromo-3,3-dimethyl-2-oxoindolin-1-yl)-1-(4-methoxy benzyl)piperidine-2,6-dione

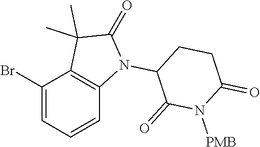

To a stirred solution of 4-bromo-3,3-dimethyl-1H-indol-2-one (2 g, 8.330 mmol) in THF (40 mL) was added t-BuOK (1.1 g, 9.996 mmol) in portions at 0° C. under nitrogen atmosphere. The resulting mixture was stirred for 1 h at 0° C. under nitrogen atmosphere. To the above mixture was added 1-[(4-methoxyphenyl)methyl]-2,6-dioxopiperidin-3-yl trifluoromethanesulfonate (4.1 g, 10.831 mmol) in THF (14 mL) dropwise at 0° C. The resulting mixture was stirred for additional 3 h at room temperature. The desired product could be detected by LCMS. The reaction was quenched with sat. NH₄Cl (aq.) (10 mL) at 0° C. and diluted with water (100 mL). The resulting mixture was extracted with EtOAc (3×150 mL). The combined organic layers were washed with brine and dried over anhydrous Na₂SO₄. After filtration, the filtrate was concentrated under reduced pressure. The residue was purified by silica gel column chromatography, eluted with PE/EtOAc (5:1 to 2:1) to afford the product (2.8 g, 71%). [M+H]⁺=471.1.

### Step 2: 3-(4-bromo-3,3-dimethyl-2-oxoindolin-1-yl)piperidine-2,6-dione

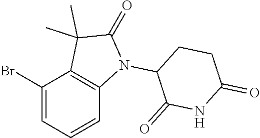 [SMILES: CC1(C)C(=O)N(C2CCC(=O)NC2=O)c2cccc(Br)c21]

A mixture of 3-(4-bromo-3,3-dimethyl-2-oxoindolin-1-yl)-1-(4-methoxybenzyl)piperidine-2,6-dione (2.8 g, 5.940 mmol) and CH₃SO₃H (8 mL, 118.731 mmol) in toluene (16 mL) was stirred for 3 h at 120° C. The desired product could be detected by LCMS. The resulting mixture was concentrated under vacuum and added to ice water. The precipitated solids were collected by filtration and washed with water. The crude product (1.2 g, 57%) was used in the next step directly without further purification. [M+H]⁺=351.0.

### Step 3: 1-(2,6-dioxopiperidin-3-yl)-3,3-dimethyl-2-oxoindoline-4-carbaldehyde

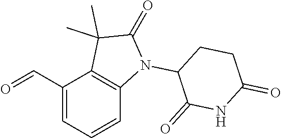 [SMILES: CC1(C)C(=O)N(C2CCC(=O)NC2=O)c2cccc(C=O)c21]

To a stirred solution of 3-(4-bromo-3,3-dimethyl-2-oxoindolin-1-yl)piperidine-2,6-dione (900 mg, 2.563 mmol) and TEA (778 mg, 7.688 mmol) in DMA (30 mL) were added Et₃SiH (894 mg, 7.688 mmol) and Pd(dppf)Cl₂ (281.26 mg, 0.384 mmol) in portions at room temperature. The resulting mixture was stirred for 16 h at 90° C. under CO atmosphere. The desired product could be detected by LCMS. The mixture was neutralized to pH 7 with AcOH and diluted with water (200 mL). The resulting mixture was extracted with EtOAc (3×100 mL). The combined organic layers were washed with brine and dried over anhydrous Na₂SO₄. After filtration, the filtrate was concentrated under reduced pressure. The residue was purified by reverse phase flash chromatography with the following conditions: Column: Spherical C18, 20-40 μm, 330 g; Mobile Phase A: Water (plus 5 mM NH₄HCO₃); Mobile Phase B: ACN; Flow rate: 80 mL/min; Gradient: 5%-5% B, 10 min, 25% B-60% B gradient in 30 min; Detector: 220 nm. The fractions containing the desired product were collected at 56% B and concentrated under reduced pressure to afford the product (121.9 mg, 16%). ¹H NMR (400 MHz, DMSO) δ 11.13 (s, 1H), 10.21 (s, 1H), 7.64 (dd, J=7.9, 1.0 Hz, 1H), 7.53 (t, J=7.8 Hz, 1H), 7.37 (d, J=7.6 Hz, 1H), 5.32 (s, 1H), 2.87 (d, J=16.9 Hz, 1H), 2.74-2.58 (m, 2H), 2.00 (d, J=11.3 Hz, 1H), 1.47 (d, J=2.9 Hz, 6H); [M−H]⁻=299.0.

### Step 4: 5-(tert-butyl)-N-((1R)-1-(4-(7-(4-((1-(2,6-dioxopiperidin-3-yl)-3,3-dimethyl-2-oxoindolin-4-yl)methyl)piperazin-1-yl)-9H-pyrimido[4,5-b]indol-4-yl)-3-fluoro-2-methylphenyl)ethyl)-1,2,4-oxadiazole-3-carboxamide

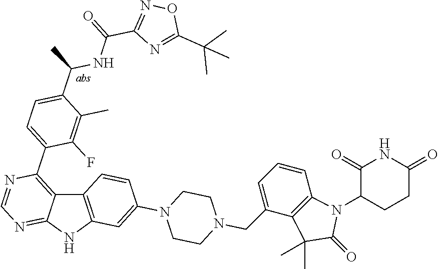 [SMILES: Cc1c([C@@H](C)NC(=O)c2noc(C(C)(C)C)n2)ccc(-c2ncnc3[nH]c4cc(N5CCN(Cc6cccc7c6C(C)(C)C(=O)N7C6CCC(=O)NC6=O)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 1-(2,6-dioxopiperidin-3-yl)-3,3-dimethyl-2-oxoindoline-4-carbaldehyde (53 mg, 0.177 mmol) and HOAc (0.06 mL) was added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-HPLC to afford the desired product (14.28 mg, 9%). ¹H NMR (500 MHz, DMSO) δ 12.19 (s, 1H), 11.07 (s, 1H), 9.59 (d, J=10.0 Hz, 1H), 8.85 (s, 1H), 7.60-7.45 (m, 2H), 7.28-7.10 (m, 3H), 6.97-6.85 (m, 3H), 5.50-5.37 (m, 1H), 5.23 (s, 1H), 3.63 (s, 2H), 3.26 (s, 4H), 2.86 (t, J=15.0 Hz, 1H), 2.69-2.56 (m, 6H), 2.39 (d, J=1.0 Hz, 3H), 1.96 (d, J=5.0 Hz, 1H), 1.56 (d, J=10.0 Hz, 3H), 1.43 (s, 15H); [M+H]⁺=841.7.

### Example 85: 5-(tert-butyl)-N-((1R)-1-(4-(7-(4-((1-(2,6-dioxopiperidin-3-yl)-3,3-dimethyl-2-oxoindolin-6-yl)methyl)piperazin-1-yl)-9H-pyrimido[4,5-b]indol-4-yl)-3-fluoro-2-methylphenyl)ethyl)-1,2,4-oxadiazole-3-carboxamide

### Step 1: 1-(tert-butyl) 3-methyl 2-(4-bromo-2-nitrophenyl)malonate

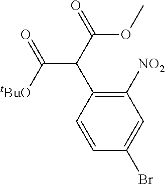 [SMILES: COC(=O)C(C(=O)OC(C)(C)C)c1ccc(Br)cc1[N+](=O)[O-]]

To a stirred solution of 1-tert-butyl 3-methyl propanedioate (30.88 g, 177.275 mmol) and 4-bromo-1-fluoro-2-nitrobenzene (32.50 g, 147.729 mmol) in DMF (200.00 mL) was added Cs₂CO₃ (96.27 g, 295.459 mmol) dropwise at 0° C. under nitrogen atmosphere. The resulting mixture was stirred for 16 h at 70° C. The resulting mixture was filtered and the filter cake was washed with EtOAc (3×500 mL). The solution was acidified to pH 7-8 with HCl (aq., 1 M). The resulting mixture was diluted with water (1.5 L) and extracted with EA (3×1000 mL). The combined organic layers were washed with brine (500 mL) and dried over anhydrous Na₂SO₄. After filtration, the filtrate was concentrated under reduced pressure. The residue was purified by silica gel column chromatography, eluted with PE/EtOAc (40:1) to afford the product (47.1 g, 85.21%). [M+H]⁺=373.9.

### Step 2: methyl 2-(4-bromo-2-nitrophenyl)acetate

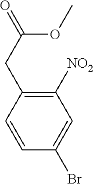

To a stirred solution of 1-(tert-butyl) 3-methyl 2-(4-bromo-2-nitrophenyl)malonate (46.10 g, 123.200 mmol) in toluene (200.00 mL) was added TsOH·H₂O (11.72 g, 61.600 mmol). The mixture was stirred at 110° C. under nitrogen atmosphere for 16 h. The resulting mixture was concentrated under vacuum and extracted with EtOAc (3×200 mL). The combined organic layers were washed with aqueous NaHCO₃ (3×100 mL) and dried over anhydrous Na₂SO₄. After filtration, the filtrate was concentrated under reduced pressure. The residue was purified by silica gel column chromatography, eluted with 1-30% EtOAc in PE to afford the product (26.5 g, 78.48%). [M+H]⁺=273.9.

### Step 3: methyl 2-(4-bromo-2-nitrophenyl)-2-methylpropanoate

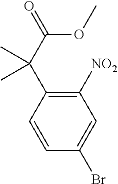 [SMILES: COC(=O)C(C)(C)c1ccc(Br)cc1[N+](=O)[O-]]

To a stirred solution of methyl 2-(4-bromo-2-nitrophenyl)acetate (12.00 g, 43.784 mmol) in ACN (240.00 mL) was added Cs₂CO₃ (28.53 g, 87.569 mmol) at 0° C. under nitrogen atmosphere at ambient temperature. To the above mixture was added CH₃I (31.07 g, 218.922 mmol) dropwise over 20 min. The resulting mixture was stirred for 16 h at 80° C. After cooling down to ambient temperature, the resulting mixture was filtered and the filtrate was washed with EtOAc (3×200 mL). The combined organic layers were concentrated under reduced pressure. The residue was purified by silica gel column chromatography, eluted with 1˜40% EtOAc in PE to afford the product (5.6 g, 42.33%). [M+H]⁺=301.9.

### Step 4: 6-bromo-3,3-dimethylindolin-2-one

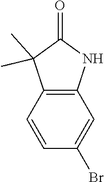

To a stirred solution of methyl 2-(4-bromo-2-nitrophenyl)-2-methylpropanoate (5.50 g, 10.923 mmol, 60%) in AcOH (50.00 mL) was added Fe (2.44 g, 43.692 mmol) at room temperature. The resulting mixture was stirred for 2 h at ambient temperature and concentrated under reduced pressure. The residue was purified by silica gel column chromatography, eluted with 10˜50% EtOAc in PE to afford 6-bromo-3,3-dimethylindolin-2-one (1.95 g, 74.36%). [M+H]⁺=239.9.

### Step 5: 3-(6-bromo-3,3-dimethyl-2-oxoindolin-1-yl)-1-(4-methoxybenzyl)piperidine-2,6-dione

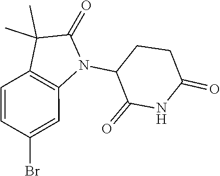 [SMILES: CC1(C)C(=O)N(C2CCC(=O)NC2=O)c2cc(Br)ccc21]

To a stirred mixture of 6-bromo-3,3-dimethylindolin-2-one (1.80 g, 7.497 mmol) in THF (160.00 mL) was added t-BuOK (0.93 g, 8.247 mmol) in portions at 0° C. under nitrogen atmosphere. The resulting mixture was stirred for 1 h at room temperature under nitrogen atmosphere. To the above mixture was added 1-[(4-methoxyphenyl)methyl]-2,6-dioxopiperidin-3-yl trifluoromethanesulfonate (3.14 g, 8.247 mmol) in THF (20 mL) at 0° C. The resulting mixture was stirred for additional 2 h at room temperature. The reaction was quenched with sat. NH₄Cl (aq.) (30 mL) at 0° C. and diluted with water (1 L). The resulting mixture was extracted with EtOAc (3×500 mL). The combined organic layers were washed with brine (2×200 mL) and dried over anhydrous Na₂SO₄. After filtration, the filtrate was concentrated under reduced pressure. The residue was purified by silica gel column chromatography, eluted with PE/EtOAc (1:1) to afford the product (2.6 g, 73.58%). [M+H]⁺=471.1.

### Step 6: 3-(6-bromo-3,3-dimethyl-2-oxoindolin-1-yl)piperidine-2,6-dione

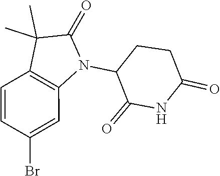 [SMILES: CC1(C)C(=O)N(C2CCC(=O)NC2=O)c2cc(Br)ccc21]

A solution of 3-(6-bromo-3,3-dimethyl-2-oxoindolin-1-yl)-1-(4-methoxybenzyl)piperidine-2,6-dione (2.60 g, 5.516 mmol) and CH₃SO₃H (10.60 g, 110.321 mmol) in toluene (260.00 mL) was stirred for 2 h at 110° C. under nitrogen atmosphere. The resulting mixture was concentrated under reduced pressure and diluted with EtOAc (400 mL). The combined organic layers were washed with brine (3×200 mL) and dried over anhydrous Na₂SO₄. After filtration, the filtrate was concentrated under reduced pressure. The residue was purified by silica gel column chromatography, eluted with 1-50% EtOAc in PE to afford the product (1.4 g, 72.27%). [M+H]⁺=351.05.

### Step 7: 1-(2,6-dioxopiperidin-3-yl)-3,3-dimethyl-2-oxoindoline-6-carbaldehyde

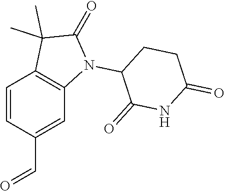 [SMILES: CC1(C)C(=O)N(C2CCC(=O)NC2=O)c2cc(C=O)ccc21]

To a stirred solution of 3-(6-bromo-3,3-dimethyl-2-oxoindolin-1-yl)piperidine-2,6-dione (1.20 g, 3.417 mmol) and Et₃SiH (1.19 g, 10.251 mmol) in DMAc (12.00 mL) were added TEA (1.04 g, 10.251 mmol) and Pd(dppf)Cl₂ (0.38 g, 0.513 mmol) at ambient temperature under nitrogen atmosphere. The resulting mixture was stirred for 16 h at 90° C. under CO atmosphere. The resulting mixture was diluted with water (100 mL) and extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with EtOAc in PE (2:1) to afford the product (110 mg, 10.72%). [M−H]⁻=299.1.

### Step 8: 5-(tert-butyl)-N-((1R)-1-(4-(7-(4-((1-(2,6-dioxopiperidin-3-yl)-3,3-dimethyl-2-oxoindolin-6-yl)methyl)piperazin-1-yl)-9H-pyrimido[4,5-b]indol-4-yl)-3-fluoro-2-methylphenyl)ethyl)-1,2,4-oxadiazole-3-carboxamide

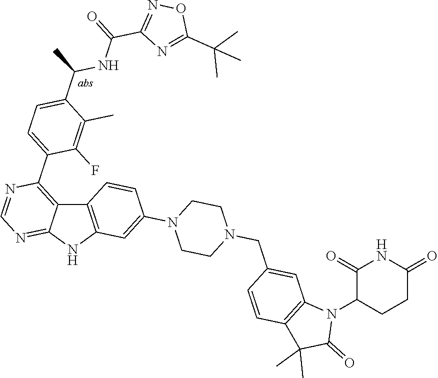 [SMILES: Cc1c([C@@H](C)NC(=O)c2noc(C(C)(C)C)n2)ccc(-c2ncnc3[nH]c4cc(N5CCN(Cc6ccc7c(c6)N(C6CCC(=O)NC6=O)C(=O)C7(C)C)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 1-(2,6-dioxopiperidin-3-yl)-3,3-dimethyl-2-oxoindoline-6-carbaldehyde (53 mg, 0.177 mmol) and HOAc (0.06 mL) were added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-HPLC to afford the desired product (2.53 mg, 1.7%). ¹H NMR (500 MHz, DMSO) δ 12.19 (s, 1H), 11.06 (s, 1H), 9.59 (d, J=10.0 Hz, 1H), 8.84 (s, 1H), 7.64-7.44 (m, 2H), 7.33 (d, J=10.0 Hz, 1H), 7.20 (d, J=5.0 Hz, 1H), 7.03 (d, J=5.0 Hz, 1H), 7.00-6.85 (m, 3H), 5.47-5.35 (m, 1H), 5.23 (s, 1H), 3.62-3.42 (m, 2H), 3.25 (s, 4H), 2.89 (t, J=15.0 Hz, 1H), 2.64-2.53 (m, 6H), 2.39 (s, 3H), 1.97 (s, 1H), 1.56 (d, J=10.0 Hz, 3H), 1.43 (s, 9H), 1.29 (d, J=5.0 Hz, 6H); [M+H]⁺=841.7.

### Example 86: 5-(tert-butyl)-N-((1R)-1-(4-(7-(4-((1-(2,6-dioxopiperidin-3-yl)-3,3-dimethyl-2-oxoindolin-5-yl)methyl)piperazin-1-yl)-9H-pyrimido[4,5-b]indol-4-yl)-3-fluoro-2-methylphenyl)ethyl)-1,2,4-oxadiazole-3-carboxamide

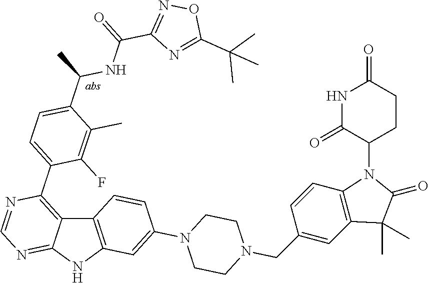 [SMILES: Cc1c([C@@H](C)NC(=O)c2noc(C(C)(C)C)n2)ccc(-c2ncnc3[nH]c4cc(N5CCN(Cc6ccc7c(c6)C(C)(C)C(=O)N7C6CCC(=O)NC6=O)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then 1-(2,6-dioxopiperidin-3-yl)-3,3-dimethyl-2-oxoindoline-5-carbaldehyde (the compound was obtained through the same way in WO2021219070A) (53 mg, 0.177 mmol) and HOAc (0.06 mL) were added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%: 0% 90%: 10% gradient elution) to give the crude product, which was further purified by prep-HPLC to afford the desired product (10.6 mg, 7%). ¹H NMR (500 MHz, DMSO) δ 12.18 (s, 1H), 11.07 (s, 1H), 9.59 (d, J=5.0 Hz, 1H), 8.84 (s, 1H), 7.59-7.39 (m, 2H), 7.34 (d, J=1.0 Hz, 1H), 7.24-7.13 (m, 2H), 7.01-6.84 (m, 3H), 5.44 (q, J=7.0 Hz, 1H), 5.23 (s, 1H), 3.50 (s, 2H), 3.24 (s, 4H), 2.95-2.85 (m, 1H), 2.68-2.52 (m, 6H), 2.38 (d, J=2.0 Hz, 3H), 2.00-1.93 (m, 1H), 1.56 (d, J=10.0 Hz, 3H), 1.43 (s, 9H), 1.30 (d, J=2.5 Hz, 6H); [M+H]⁺=841.7.

### Example 87: 5-(tert-butyl)-N-((1R)-1-(4-(7-(4-(1-((3-(2,6-dioxopiperidin-3-yl)-1-methyl-2-oxo-2,3-dihydro-1H-benzo[d]imidazol-5-yl)methyl)piperidin-4-yl)piperazin-1-yl)-9H-pyrimido[4,5-b]indol-4-yl)-3-fluoro-2-methylphenyl)ethyl)-1,2,4-oxadiazole-3-carboxamide

### Step 1: tert-butyl (R)-4-(4-(44-(1-(5-(tert-butyl)-1,2,4-oxadiazole-3-carboxamido)ethyl)-2-fluoro-3-methylphenyl)-9H-pyrimido[4,5-b]indol-7-yl)piperazin-1-yl)piperidine-1-carboxylate

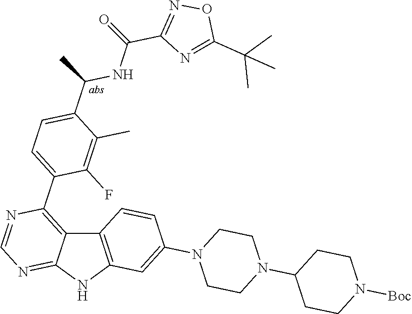 [SMILES: Cc1c([C@@H](C)NC(=O)c2noc(C(C)(C)C)n2)ccc(-c2ncnc3[nH]c4cc(N5CCN(C6CCN(C(=O)OC(C)(C)C)CC6)CC5)ccc4c23)c1F]

To a solution of (R)-5-(tert-butyl)-N-(1-(3-fluoro-2-methyl-4-(7-(piperazin-1-yl)-9H-pyrimido[4,5-b]indol-4-yl)phenyl)ethyl)-1,2,4-oxadiazole-3-carboxamide (100 mg, crude) in dichloromethane (5 mL) and MeOH (5 mL), NaOAc (43.5 mg, 0.531 mmol) was added. The mixture was stirred at room temperature for 5 mins. Then tert-butyl 4-oxopiperidine-1-carboxylate (71 mg, 0.354 mmol) and HOAc (0.06 mL) were added. The mixture was stirred at room temperature overnight. Then NaBH(OAc)₃ (187.6 mg, 0.885 mmol) was added and stirred at room temperature overnight. Then NaBH₃CN (114.8 mg, 1.8 mmol) was added and stirred at room temperature overnight. The reaction mixture was concentrated in vacuum, and the residue was purified by silica gel column chromatography (DCM:MeOH=100%:0%˜90%:10% gradient elution) to give the product (97 mg, 73%). [M+H]⁺=740.5.

### Step 2: (R)-5-(tert-butyl)-N-(1-(3-fluoro-2-methyl-4-(7-(4-(piperidin-4-yl)piperazin-1-yl)-9H-pyrimido[4,5-b]indol-4-yl)phenyl)ethyl)-1,2,4-oxadiazole-3-carboxamide

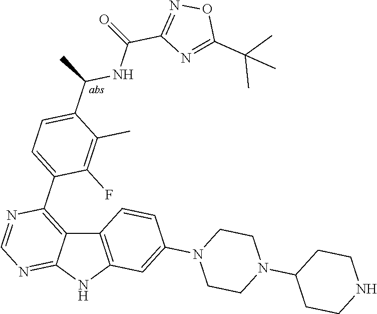 [SMILES: Cc1c([C@@H](C)NC(=O)c2noc(C(C)(C)C)n2)ccc(-c2ncnc3[nH]c4cc(N5CCN(C6CCNCC6)CC5)ccc4c23)c1F]

A mixture of tert-butyl (R)-4-(4-(4-(4-(1-(5-(tert-butyl)-1,2,4-oxadiazole-3-carboxamido)ethyl)-2-fluoro-3-methylphenyl)-9H-pyrimido[4,5-b]indol-7-yl)piperazin-1-yl)piperidine-1-carboxylate (97 mg, 0.131 mmol) in 4 M HCl in 1,4-dioxane (10 mL) was stirred in a round bottom flask at room temperature overnight. The mixture was evaporated in vacuum to afford the product (78 mg, 93%). [M+H]⁺=640.5.

### Step 3: 5-(tert-butyl)-N-((1R)-1-(4-(7-(4-(1-((3-(2,6-dioxopiperidin-3-yl)-1-methyl-2-oxo-2,3-dihydro-1H-benzo[d]imidazol-5-yl)methyl)piperidin-4-yl)piperazin-1-yl)-9H-pyrimido[4,5-b]indol-4-yl)-3-fluoro-2-methylphenyl)ethyl)-1,2,4-oxadiazole-3-carboxamide

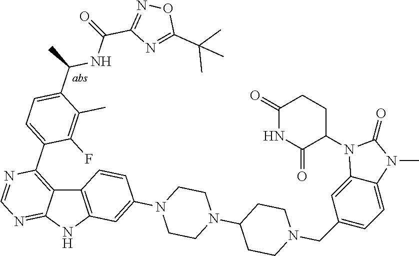 [SMILES: Cc1c([C@@H](C)NC(=O)c2noc(C(C)(C)C)n2)ccc(-c2ncnc3[nH]c4cc(N5CCN(C6CCN(Cc7ccc8c(c7)n(C7CCC(=O)NC7=O)c(=O)n8C)CC6)CC5)ccc4c23)c1F]

To a solution of (R)-5-(tert-butyl)-N-(1-(3-fluoro-2-methyl-4-(7-(4-(piperidin-4-yl)piperazin-1-yl)-9H-pyrimido[4,5-b]indol-4-yl)phenyl)ethyl)-1,2,4-oxadiazole-3-carboxamide (78 mg, 0.122 mmol) in dichloromethane (5 mL) and MeOH (5 mL), NaOAc (30 mg, 0.366 mmol) was added. The mixture was stirred at room temperature for 5 mins. Then 3-(2,6-dioxopiperidin-3-yl)-1-methyl-2-oxo-2,3-dihydro-1H-benzo[d]imidazole-5-carbaldehyde (42 mg, 0.146 mmol) and HOAc (0.06 mL) were added. The mixture was stirred at room temperature overnight. Then NaBH(OAc)₃ (129 mg, 0.610 mmol) was added and stirred at room temperature overnight. Then NaBH₃CN (78 mg, 1.22 mmol) was added and stirred at room temperature overnight. The reaction mixture was concentrated in vacuum, and the residue was purified by silica gel column chromatography (DCM:MeOH=100%:0%˜90%:10% gradient elution) to give the crude product, which was further purified by prep-HPLC to afford the desired product (8.92 mg, 8%). ¹H NMR (500 MHz, DMSO) δ 12.18 (s, 1H), 11.10 (s, 1H), 9.59 (d, J=10.0 Hz, 1H), 8.84 (s, 1H), 8.20 (s, 1H), 7.59-7.39 (m, 2H), 7.24-6.98 (m, 4H), 6.97-6.82 (m, 2H), 5.50-5.24 (m, 2H), 3.45 (s, 2H), 3.30-3.27 (m, 2H), 3.21 (s, 1H), 2.96-2.82 (m, 3H), 2.77-2.60 (m, 6H), 2.54 (s, 2H), 2.39 (d, J=1.7 Hz, 3H), 2.21 (t, J=11.1 Hz, 1H), 2.06-1.98 (m, 1H), 1.92 (t, J=9.8 Hz, 2H), 1.75 (d, J=10.6 Hz, 2H), 1.56 (d, J=6.9 Hz, 3H), 1.43 (s, 11H); [M+H]⁺=911.7.

### Example 88: 5-(tert-butyl)-N-((1R)-1-(4-(7-(4-(4-((2,6-dioxopiperidin-3-yl)thio)benzyl)piperazin-1-yl)-9H-pyrimido[4,5-b]indol-4-yl)-3-fluoro-2-methylphenyl)ethyl)-1,2,4-oxadiazole-3-carboxamide

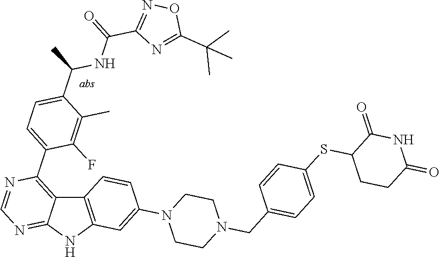 [SMILES: Cc1c([C@@H](C)NC(=O)c2noc(C(C)(C)C)n2)ccc(-c2ncnc3[nH]c4cc(N5CCN(Cc6ccc(SC7CCC(=O)NC7=O)cc6)CC5)ccc4c23)c1F]

The titled compound was synthesized in the procedures similar to Example 80. ¹H NMR (500 MHz, DMSO) δ 12.19 (s, 1H), 10.90 (s, 1H), 9.59 (d, J=10.0 Hz, 1H), 8.85 (s, 1H), 7.60-7.42 (m, 5H), 7.33 (d, J=5.0 Hz, 2H), 7.20 (d, J=81.0 Hz, 1H), 6.95-6.88 (m, 2H), 5.47-5.34 (m, 1H), 4.34-4.24 (m, 1H), 3.65-3.58 (m, 2H), 3.53 (s, 2H), 3.24-3.19 (m, 2H), 3.18-3.09 (m, 2H), 2.58-2.53 (m, 2H), 2.39 (d, J=1.5 Hz, 3H), 2.27-2.16 (m, 2H), 2.00-1.89 (m, 1H), 1.56 (d, J=6.5 Hz, 3H), 1.43 (s, 9H); [M+H]⁺=790.6.

### Example 89: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-5-fluoro-2-methylphenyl)ethyl)-3-(1-methylcyclopropyl)-1,2,4-oxadiazole-5-carboxamide

### Step 1: (S)—N-(4-bromo-5-fluoro-2-methylbenzylidene)-2-methylpropane-2-sulfinamide

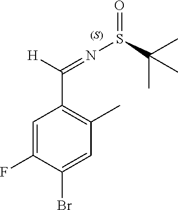

To a stirred mixture of 4-bromo-5-fluoro-2-methylbenzaldehyde (50 g, 230.375 mmol) and Ti(Oi-Pr)₄ (261.9 g, 921.485 mmol) in THF (5 mL) was added (S)-2-methylpropane-2-sulfinamide (36.3 g, 299.505 mmol) in portions at room temperature. The resulting mixture was stirred for 1.5 h at 50° C. The mixture was allowed to cool down to 15° C. The reaction was quenched with sat. NaCl (aq.) (200 mL) and EtOAc (200 mL) at room temperature. The resulting mixture was filtered, and the filter cake was washed with EtOAc (2×200 mL). The filtrate was concentrated under reduced pressure. The resulting mixture was extracted with EtOAc (2×200 mL). The combined organic layers were washed with brine (2×100 mL) and dried over anhydrous Na₂SO₄. After filtration, the filtrate was concentrated under reduced pressure. The residue was purified by silica gel column chromatography, eluted with PE/EtOAc (3:1) to afford the product (68.9 g, 93%). [M+H]⁺=320.0.

### Step 2: (S)—N—((R)-1-(4-bromo-5-fluoro-2-methylphenyl)ethyl)-2-methylpropane-2-sulfinamide

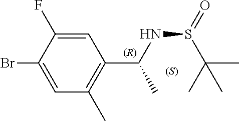

To a stirred solution of (S)—N-(4-bromo-5-fluoro-2-methylbenzylidene)-2-methylpropane-2-sulfinamide (68.9 g, 215.165 mmol) in DCM (690 mL) was added MeMgBr (74 mL, 645.508 mmol) dropwise at −50° C. under nitrogen atmosphere. The resulting mixture was stirred for 16 h at room temperature under nitrogen atmosphere. The reaction was quenched with sat. NH₄Cl at 0° C. The resulting mixture was extracted with EtOAc (3×200 mL). The combined organic layers were washed with brine (2×100 mL) and dried over anhydrous Na₂SO₄. After filtration, the filtrate was concentrated under reduced pressure. The residue was purified by silica gel column chromatography, eluted with PE/EtOAc (1:1) to afford the product (50 g, 68%). [M+H]⁺=335.9.

### Step 3: (S)—N—((R)-1-(5-fluoro-2-methyl-4-(4,4,5,5-tetramethyl-1,3,2-dioxaborolan-2-yl)phenyl)ethyl)-2-methylpropane-2-sulfinamide

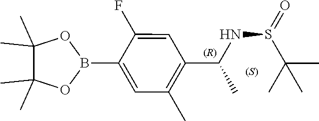

To a stirred mixture of (S)—N—((R)-1-(4-bromo-5-fluoro-2-methylphenyl)ethyl)-2-methylpropane-2-sulfinamide (25 g, 74.347 mmol) and bis(pinacolato)diboron (37.7 g, 148.694 mmol) in dioxane (120 mL) were added Pd(dppf)Cl₂ (5.4 g, 7.435 mmol) and KOAc (14.6 g, 148.694 mmol) at room temperature. The resulting mixture was stirred for 16 h at 80° C. under nitrogen atmosphere. The resulting mixture was allowed to cool down to ambient temperature and diluted with water (800 mL). The resulting mixture was extracted with CH₂Cl₂ (3×300 mL). The combined organic layers were washed with brine (2×100 mL) and dried over anhydrous Na₂SO₄. After filtration, the filtrate was concentrated under reduced pressure. The residue was purified by silica gel column chromatography, eluted with CH₂Cl₂/EtOAc (10:1) to afford the product (19 g, 66%). [M+H]⁺=384.1.

### Step 4: (R)-1-(5-fluoro-2-methyl-4-(4,4,5,5-tetramethyl-1,3,2-dioxaborolan-2-yl)phenyl)ethan-1-amine hydrochloride

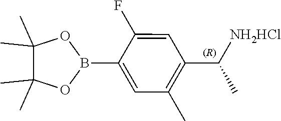

To a stirred solution of (S)—N—((R)-1-(5-fluoro-2-methyl-4-(4,4,5,5-tetramethyl-1,3,2-dioxaborolan-2-yl)phenyl)ethyl)-2-methylpropane-2-sulfinamide (18.9 g, 49.305 mmol) in DCM (160 mL) was added 4 M HCl in 1,4-dioxane (160 mL) dropwise at room temperature. The resulting mixture was stirred for 30 min at room temperature and concentrated under vacuum. The resulting mixture was diluted with MTBE (I50 mL) and stirred for 30 min at room temperature. The resulting mixture was filtered and the filter cake was washed with MTBE (2×60 mL) to afford the product (14.1 g, 91%). ¹H NMR (400 MHz, DMSO) δ 8.49 (s, 3H), 7.48 (d, J=6.2 Hz, 1H), 7.37 (d, J=10.5 Hz, 1H), 4.53 (q, J=6.7 Hz, 1H), 2.32 (s, 3H), 1.45 (d, J=6.8 Hz, 3H), 1.30 (s, 12H); [M+H]⁺=280.3.

### Step 5: (R)—N-(1-(5-fluoro-2-methyl-4-(4,4,5,5-tetramethyl-1,3,2-dioxaborolan-2-yl)phenyl)ethyl)-3-(1-methylcyclopropyl)-1,2,4-oxadiazole-5-carboxamide

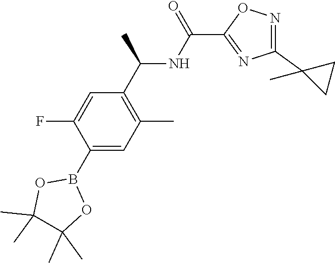 [SMILES: Cc1cc(B2OC(C)(C)C(C)(C)O2)c(F)cc1[C@@H](C)NC(=O)c1nc(C2(C)CC2)no1]

To a solution of (R)-1-(5-fluoro-2-methyl-4-(4,4,5,5-tetramethyl-1,3,2-dioxaborolan-2-yl)phenyl)ethan-1-amine hydrochloride (3.49 g, 0.0129 mmol) in MeOH (20 mL), NH₃ (7 M in MeOH, 3 mL) was added. Then the mixture was concentrated in vacuo. Then the residue was diluted with EtOH (40 mL) and ethyl 3-(1-methylcyclopropyl)-1,2,4-oxadiazole-5-carboxylate (2.8 g, 0.0143 mol) was added. The mixture was stirred at 80° C. overnight. The mixture was concentrated in vacuo, and the residue was purified with silica gel column chromatography (PE:EA=100:0˜70:30 gradient elution) to give the titled product (840 mg, 15%). [M+H]⁺=430.3.

### Step 6: benzyl (R)-4-(4-(2-fluoro-5-methyl-4-(1-(3-(1-methylcyclopropyl)-1,2,4-oxadiazole-5-carboxamido)ethyl)phenyl)-9H-pyrimido[4,5-b]indol-7-yl)piperazine-1-carboxylate

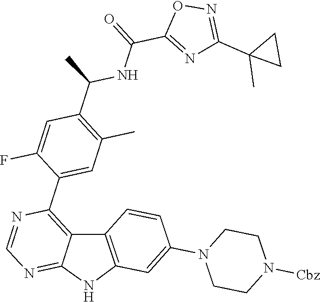 [SMILES: Cc1cc(-c2ncnc3[nH]c4cc(N5CCN(C(=O)OCc6ccccc6)CC5)ccc4c23)c(F)cc1[C@@H](C)NC(=O)c1nc(C2(C)CC2)no1]

A mixture of benzyl 4-(4-chloro-9H-pyrimido[4,5-b]indol-7-yl)piperazine-1-carboxylate (421 mg, 1 mmol), (R)—N-(1-(5-fluoro-2-methyl-4-(4,4,5,5-tetramethyl-1,3,2-dioxaborolan-2-yl)phenyl)ethyl)-3-(1-methylcyclopropyl)-1,2,4-oxadiazole-5-carboxamide (429 mg, 1 mmol), Pd(dppf)Cl₂ (36.5 mg, 0.05 mmol) and K₂CO₃ (276 mg, 2 mmol) in 1,4-dioxane (15 mL) and water (4 mL) was stirred at 93° C. overnight. The mixture was allowed to cool and evaporated in vacuum to afford the crude product, which was further purified with silica gel column chromatography (PE:EA=100:0˜20:80 gradient elution) to give the titled product (350 mg, 50%). [M+H]⁺=689.5.

### Step 7: (R)—N-(1-(5-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide

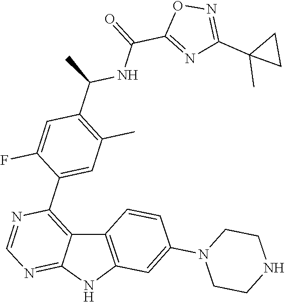 [SMILES: Cc1cc(-c2ncnc3[nH]c4cc(N5CCNCC5)ccc4c23)c(F)cc1[C@@H](C)NC(=O)c1nc(C2(C)CC2)no1]

A mixture of benzyl (R)-4-(4-(2-fluoro-5-methyl-4-(1-(3-(1-methylcyclopropyl)-1,2,4-oxadiazole-5-carboxamido)ethyl)phenyl)-9H-pyrimido[4,5-b]indol-7-yl)piperazine-1-carboxylate (350 mg, 2.17 mmol) in trifluoroacetic acid (8 mL) was stirred at 85° C. for 3 hours. After the reaction was cooled to ambient temperature, the mixture was concentrated in vacuum. The mixture was diluted with DCM, and NH₃ (7 M in MeOH) (2 mL) was added. The mixture was stirred at room temperature for 5 mins and concentrated in vacuo to afford the product (1.3 g, crude), which was used in next step without further purification. [M+H]⁺=555.5.

### Step 8: (R)—N-(1-(4-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-5-fluoro-2-methylphenyl)ethyl)-3-(1-methylcyclopropyl)-1,2,4-oxadiazole-5-carboxamide

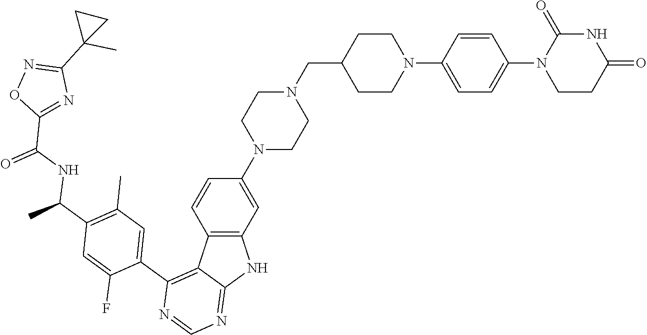 [SMILES: Cc1cc(-c2ncnc3[nH]c4cc(N5CCN(CC6CCN(c7ccc(N8CCC(=O)NC8=O)cc7)CC6)CC5)ccc4c23)c(F)cc1[C@@H](C)NC(=O)c1nc(C2(C)CC2)no1]

To a solution of (R)—N-(1-(5-fluoro-2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (200 mg, crude) in dichloromethane (6 mL) and MeOH (6 mL), 1-(4-(2,4-dioxotetrahydropyrimidin-1(2H)-yl)phenyl)piperidine-4-carbaldehyde (130 mg, 0.433 mmol) and HOAc (0.06 mL) were added. The mixture was stirred at room temperature overnight. Then NaBH(OAc)₃ (382 mg, 1.805 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the crude product, which was further purified by prep-TLC (DCM:MeOH=10:1) to afford the desired product ((26.49 mg, 8.9%). ¹H NMR (500 MHz, DMSO) δ 12.20 (s, 1H), 10.25 (s, 1H), 9.84 (d, J=10.0 Hz, 1H), 8.85 (s, 1H), 7.56-7.47 (m, 2H), 7.31-7.20 (m, 1H), 7.13 (d, J=10.0 Hz, 2H), 6.96-6.86 (m, 4H), 5.42-5.28 (m, 1H), 3.69 (t, J=6.5 Hz, 4H), 3.25 (s, 4H), 2.71-2.62 (m, 4H), 2.53 (s, 4H), 2.44 (s, 3H), 2.23 (d, J=10.0 Hz, 2H), 1.81 (d, J=15.0 Hz, 2H), 1.76-1.68 (m, 1H), 1.56 (d, J=5.0 Hz, 3H), 1.49 (s, 3H), 1.27-1.19 (m, 4H), 1.00 (t, J=3.8 Hz, 2H); [M+H]⁺=840.7.

### Example 90: N—((R)-1-(4-(7-(4-(3-(4-((S)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-3-(1-methylcyclopropyl)-1,2,4-oxadiazole-5-carboxamide

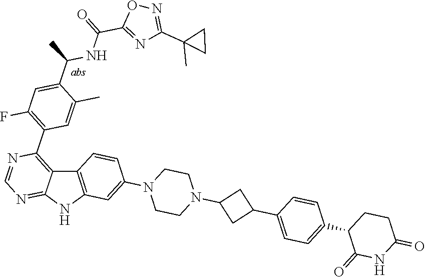 [SMILES: Cc1cc(-c2ncnc3[nH]c4cc(N5CCN(C6CC(c7ccc([C@@H]8CCC(=O)NC8=O)cc7)C6)CC5)ccc4c23)c(F)cc1[C@@H](C)NC(=O)c1nc(C2(C)CC2)no1]

The titled compound was synthesized in the procedures similar to Example 89. ¹H NMR (500 MHz, DMSO) δ 12.20 (s, 1H), 10.81 (s, 1H), 9.84 (d, J=10.0 Hz, 1H), 8.85 (s, 1H), 7.56-7.50 (m, 2H), 7.30-7.10 (m, 5H), 6.95-6.90 (m, 2H), 5.45-5.30 (m, 1H), 3.85-3.79 (m, 1H), 3.32 (s, 6H), 3.19-3.08 (m, 1H), 2.80-2.72 (m, 1H), 2.71-2.63 (m, 1H), 2.49-2.42 (m, 8H), 2.22-2.12 (m, 1H), 2.07-1.99 (m, 1H), 1.93-1.83 (m, 2H), 1.56 (d, J=5.0 Hz, 3H), 1.49 (s, 3H), 1.26-1.18 (m, 2H), 1.00 (d, J=5.0 Hz, 2H); [M+H]⁺=796.6.

### Example 91: 5-(tert-butyl)-N-((1R)-1-(4-(7-(4-((1′-(2,6-dioxopiperidin-3-yl)-2′-oxospiro[cyclopropane-1,3′-indolin]-6′-yl)methyl)piperazin-1-yl)-9H-pyrimido[4,5-b]indol-4-yl)-5-fluoro-2-methylphenyl)ethyl)-1,2,4-oxadiazole-3-carboxamide

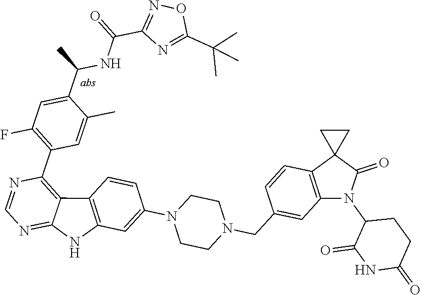 [SMILES: Cc1cc(-c2ncnc3[nH]c4cc(N5CCN(Cc6ccc7c(c6)N(C6CCC(=O)NC6=O)C(=O)C76CC6)CC5)ccc4c23)c(F)cc1[C@@H](C)NC(=O)c1noc(C(C)(C)C)n1]

The titled compound was synthesized in the procedures similar to Example 89. ¹H NMR (500 MHz, DMSO) δ 12.19 (s, 1H), 11.08 (s, 1H), 9.53 (d, J=10.0 Hz, 1H), 8.84 (d, J=5.0 Hz, 1H), 7.60-7.40 (m, 2H), 7.25 (d, J=10.0 Hz, 1H), 7.00 (s, 3H), 6.94-6.87 (m, 2H), 5.43-5.29 (m, 2H), 3.53 (s, 2H), 3.24 (s, 4H), 2.88 (t, J=15.0 Hz, 1H), 2.70-2.53 (m, 6H), 2.45 (s, 3H), 1.98 (s, 1H), 1.63 (s, 2H), 1.55 (d, J=5.0 Hz, 4H), 1.43 (s, 9H); [M+H]⁺=839.5.

### Example 92: 3-(tert-butyl)-N-((1R)-1-(4-(6-methoxy-7-(4-(4-(3-methyl-2,6-dioxopiperidin-3-yl)phenethyl)piperazin-1-yl)-9H-pyrimido[4,5-b]indol-4-yl)-2-methylphenyl)ethyl)-1,2,4-oxadiazole-5-carboxamide

### Step 1: (E)-3-(4-(2-ethoxyvinyl)phenyl)-3-methylpiperidine-2,6-dione

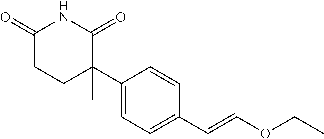 [SMILES: CCO/C=C/c1ccc(C2(C)CCC(=O)NC2=O)cc1]

To a stirred mixture of 3-(4-bromophenyl)-3-methylpiperidine-2,6-dione (670 mg, 2.38 mmol) and (E)-2-(2-ethoxyvinyl)-4,4,5,5-tetramethyl-1,3,2-dioxaborolane (566.5 mg, 2.86 mmol) in DMF/H₂O (12.0 mL/3.0 mL) were added Pd(dtbpf)Cl₂ (77.6 mg, 0.119 mmol) and CsF (723.5 mg, 4.76 mmol). After stirring for 3 h at 80° C. under a nitrogen atmosphere, the resulting mixture was concentrated under reduced pressure. The residue was purified by silica gel column chromatography, eluted with PE/EtOAc (6:1 to 1:1) to afford the product (560 mg, 86.1%). [M+H]⁺=274.2.

### Step 2: 2-(4-(3-methyl-2,6-dioxopiperidin-3-yl)phenyl)acetaldehyde

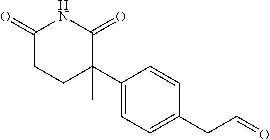 [SMILES: CC1(c2ccc(CC=O)cc2)CCC(=O)NC1=O]

A solution of (E)-3-(4-(2-ethoxyvinyl)phenyl)-3-methylpiperidine-2,6-dione (560 mg, 2.05 mmol) in FA (20.0 mL) was stirred for 16 h at room temperature, and the resulting mixture was evaporated under reduced pressure. The residue was purified by silica gel column chromatography, eluted with PE/EtOAc (4:1 to 1:3) to afford the product (200 mg, 40%). [M+H]⁺=246.1.

### Step 3: 3-(tert-butyl)-N-((1R)-1-(4-(6-methoxy-7-(4-(4-(3-methyl-2,6-dioxopiperidin-3-yl)phenethyl)piperazin-1-yl)-9H-pyrimido[4,5-b]indol-4-yl)-2-methylphenyl)ethyl)-1,2,4-oxadiazole-5-carboxamide

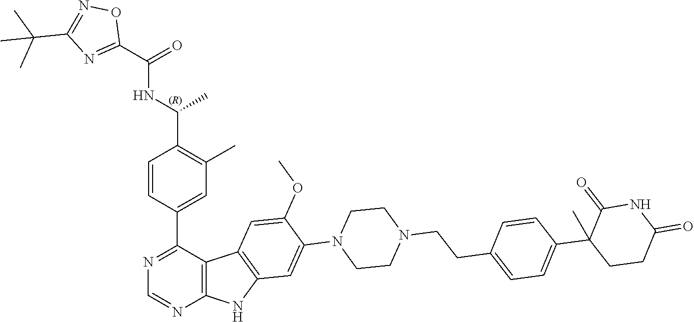

A solution of (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide formate (100 mg, 0.163 mmol), 2-(4-(3-methyl-2,6-dioxopiperidin-3-yl)phenyl)acetaldehyde (60 mg, 0.244 mmol) and Ti(i-PrO)₄ (10 drops) in THF (4.0 mL) and DMF (2.0 mL) was stirred for 16 h at 25° C., then NaBH(OAc)₃ (172.8 mg, 0.815 mmol) was added and stirred at room temperature for 2 h. The resulting mixture was diluted with water (10.0 mL) and filtered. The residue was purified by prep-HPLC to afford the product (20 mg, 15.4%). ¹H NMR (500 MHz, DMSO) δ 13.55-13.48 (m, 1H), 12.14 (s, 1H), 10.90 (s, 1H), 9.93 (d, J=7.7 Hz, 1H), 8.85 (s, 1H), 7.83-7.75 (m, 2H), 7.72 (d, J=8.0 Hz, 1H), 7.27 (d, J=9.5 Hz, 3H), 7.20 (d, J=8.3 Hz, 2H), 7.01 (s, 1H), 5.40 (q, J=6.9 Hz, 1H), 3.66 (s, 3H), 3.09 (s, 4H), 2.80-2.73 (m, 2H), 2.63 (s, 3H), 2.61-2.56 (m, 2H), 2.53 (s, 3H), 2.45 (d, J=13.9 Hz, 2H), 2.36 (dd, J=13.0, 4.7 Hz, 1H), 2.12-2.00 (m, 2H), 1.58 (d, J=7.0 Hz, 3H), 1.43 (s, 3H), 1.37 (s, 9H); [M+H]⁺=798.6.

### Example 93: (R)—N-(1-(4-(7-(4-((1-(4-(2,4-dioxotetrahydropyrimidin-1(2H)-yl)phenyl)piperidin-4-yl)methyl)piperazin-1-yl)-6-methoxy-9H-pyrimido[4,5-b]indol-4-yl)-5-fluoro-2-methylphenyl)ethyl)-3-(1-methylcyclopropyl)-1,2,4-oxadiazole-5-carboxamide

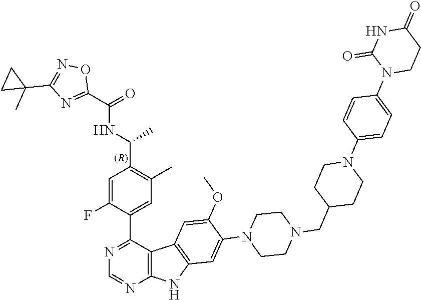

The titled compound was synthesized in the procedures similar to Example 47. ¹H NMR (500 MHz, DMSO) δ 12.21 (s, 1H), 10.25 (s, 1H), 9.87 (d, J=7.7 Hz, 1H), 8.89 (s, 1H), 7.58-7.49 (m, 2H), 7.13 (t, J=8.0 Hz, 2H), 7.02 (s, 1H), 6.94 (d, J=8.3 Hz, 2H), 6.78 (s, 1H), 5.39-5.28 (m, 1H), 3.69 (t, J=6.7 Hz, 4H), 3.55 (s, 3H), 3.15-3.02 (m, 3H), 2.69-2.64 (m, 4H), 2.60-2.52 (m, 2H), 2.46 (s, 3H), 2.28-2.19 (m, 1H), 1.87-1.78 (m, 2H), 1.77-1.67 (m, 1H), 1.56 (d, J=6.9 Hz, 3H), 1.29-1.18 (m, 9H), 1.03-0.98 (m, 2H), 0.88-0.79 (m, 2H); [M+H]⁺=870.3.

### Example 94: N—((R)-1-(4-(7-(4-((1S,3s)-3-(4-(((R)-2,6-dioxopiperidin-3-yl)amino)phenyl)cyclobutyl)piperazin-1-yl)-6-methoxy-9H-pyrimido[4,5-b]indol-4-yl)-5-fluoro-2-methylphenyl)ethyl)-3-(1-methylcyclopropyl)-1,2,4-oxadiazole-5-carboxamide

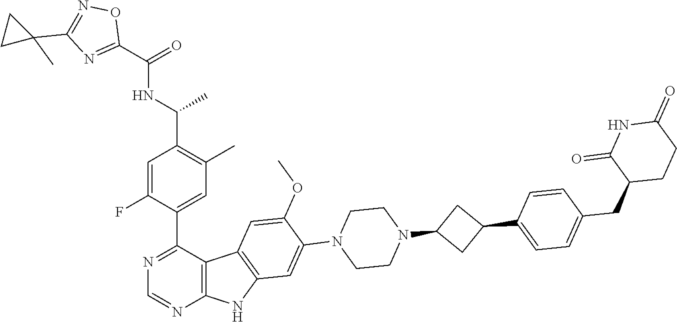 [SMILES: COc1cc2c(cc1N1CCN([C@H]3C[C@@H](c4ccc(C[C@@H]5CCC(=O)NC5=O)cc4)C3)CC1)[nH]c1ncnc(-c3cc(C)c([C@@H](C)NC(=O)c4nc(C5(C)CC5)no4)cc3F)c12]

The titled compound was synthesized in the procedures similar to Example 47. ¹H NMR (500 MHz, DMSO) δ 12.12 (s, 1H), 10.70 (s, 1H), 9.79 (d, J=7.7 Hz, 1H), 8.81 (s, 1H), 8.24 (s, 1H), 7.48-7.43 (m, 2H), 6.99-6.93 (m, 3H), 6.71 (s, 1H), 6.58 (d, J=8.6 Hz, 2H), 5.61 (d, J=7.5 Hz, 1H), 5.31-5.21 (m, 1H), 4.23-4.19 (m, 1H), 3.49-3.44 (m, 4H), 3.02 (s, 3H), 2.88-2.80 (m, 2H), 2.70-2.64 (m, 2H), 2.58-2.47 (m, 2H), 2.39 (s, 3H), 3.30-2.21 (m, 3H), 2.07-1.98 (m, 3H), 1.82-1.78 (m, 1H), 1.49 (d, J=6.9 Hz, 3H), 1.43 (s, 3H), 1.20-1.10 (m, 2H), 0.95-0.91 (m, 2H); [M+H]⁺=841.3.

### Example 95: N—((R)-1-(4-(7-(4-((1R,3s)-3-(4-(((S)-2,6-dioxopiperidin-3-yl)amino)phenyl)cyclobutyl)piperazin-1-yl)-6-methoxy-9H-pyrimido[4,5-b]indol-4-yl)-5-fluoro-2-methylphenyl)ethyl)-3-(1-methylcyclopropyl)-1,2,4-oxadiazole-5-carboxamide

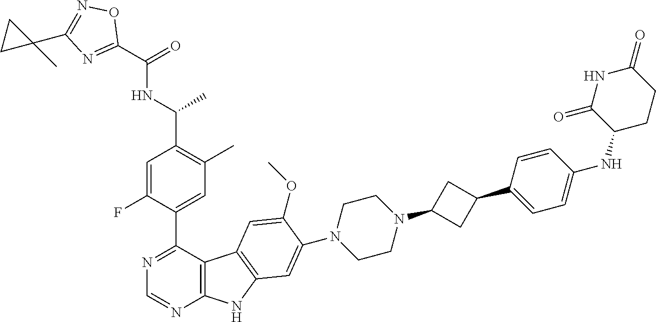 [SMILES: COc1cc2c(cc1N1CCN([C@H]3C[C@@H](c4ccc(N[C@H]5CCC(=O)NC5=O)cc4)C3)CC1)[nH]c1ncnc(-c3cc(C)c([C@@H](C)NC(=O)c4nc(C5(C)CC5)no4)cc3F)c12]

The titled compound was synthesized in the procedures similar to Example 47. ¹H NMR (500 MHz, DMSO) δ 12.11 (s, 1H), 10.70 (s, 1H), 9.78 (d, J=7.7 Hz, 1H), 8.81 (s, 1H), 8.18 (s, 1H), 7.51-7.37 (m, 2H), 6.94 (s, 1H), 6.90 (d, J=8.5 Hz, 2H), 6.70 (s, 1H), 6.55 (d, J=8.5 Hz, 2H), 5.60 (d, J=7.6 Hz, 1H), 5.28-5.24 (m, 1H), 4.25-4.17 (m, 1H), 3.48 (s, 3H), 3.07-2.86 (m, 6H), 2.72-2.46 (m, 4H), 2.39 (s, 3H), 2.36-2.26 (m, 3H), 2.07-1.99 (m, 1H), 1.84-1.68 (m, 3H), 1.49 (d, J=7.0 Hz, 3H), 1.43 (s, 3H), 1.18-1.11 (m, 2H), 0.96-0.90 (m, 2H); [M+H]⁺=841.3.

### Example 96: N—((R)-1-(4-(7-(4-(3-(4-((R)-2,6-dioxopiperidin-3-yl)phenyl)cyclobutyl)piperazin-1-yl)-9H-pyrimido[4,5-b]indol-4-yl)-5-fluoro-2-methylphenyl)ethyl)-3-(1-methylcyclopropyl)-1,2,4-oxadiazole-5-carboxamide

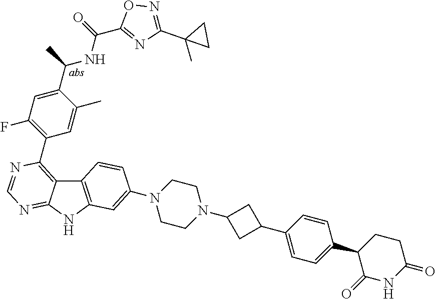 [SMILES: Cc1cc(-c2ncnc3[nH]c4cc(N5CCN(C6CC(c7ccc([C@H]8CCC(=O)NC8=O)cc7)C6)CC5)ccc4c23)c(F)cc1[C@@H](C)NC(=O)c1nc(C2(C)CC2)no1]

The titled compound was synthesized in the procedures similar to Example 89. ¹H NMR (500 MHz, DMSO) δ 12.20 (s, 1H), 10.81 (s, 1H), 9.84 (s, 1H), 8.92-8.76 (m, 1H), 7.53 (s, 2H), 7.28-7.12 (m, 5H), 6.92 (s, 2H), 5.37 (s, 1H), 3.82 (d, J=5.0 Hz, 1H), 3.32 (s, 6H), 3.14 (s, 2H), 2.75 (s, 1H), 2.69-2.62 (m, 1H), 2.48-2.41 (m, 8H), 2.18 (s, 1H), 2.03 (s, 1H), 1.88 (s, 2H), 1.56 (s, 3H), 1.49 (s, 3H), 1.21 (s, 2H), 0.99 (s, 2H); [M+H]⁺=796.6.

### Example 97: 5-(tert-butyl)-N-((1R)-1-(4-(7-(4-((1-(4-(2,6-dioxopiperidin-3-yl)phenyl)piperidin-4-yl)methyl)piperazin-1-yl)-9H-pyrimido[4,5-b]indol-4-yl)-5-fluoro-2-methylphenyl)ethyl)-1,2,4-oxadiazole-3-carboxamide

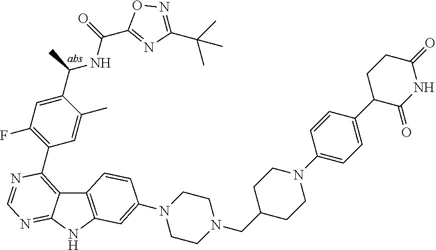 [SMILES: Cc1cc(-c2ncnc3[nH]c4cc(N5CCN(CC6CCN(c7ccc(C8CCC(=O)NC8=O)cc7)CC6)CC5)ccc4c23)c(F)cc1[C@@H](C)NC(=O)c1nc(C(C)(C)C)no1]

The titled compound was synthesized in the procedures similar to Example 89. ¹H NMR (500 MHz, DMSO) δ 12.20 (s, 1H), 10.25 (s, 1H), 9.53 (d, J=10.0 Hz, 1H), 8.85 (d, J=1.5 Hz, 1H), 7.59-7.46 (m, 2H), 7.26 (d, J=10.0 Hz, 1H), 7.13 (d, J=5.0 Hz, 2H), 6.92 (s, 4H), 5.44-5.34 (m, 1H), 3.73-3.63 (m, 4H), 3.29-3.20 (m, 5H), 2.67 (dd, J=14.1, 9.8 Hz, 4H), 2.70-2.62 (m, 4H), 2.53 (s, 3H), 2.45 (s, 4H), 2.24 (s, 2H), 1.81 (d, J=12.5 Hz, 2H), 1.72 (s, 1H), 1.55 (d, J=6.4 Hz, 3H), 1.44 (d, J=1.5 Hz, 9H), 1.26-1.19 (m, 2H); [M+H]⁺=842.6.

### Example 98: 5-(tert-butyl)-N-((1R)-1-(4-(7-(4-((1-((1-(2,6-dioxopiperidin-3-yl)-3,3-dimethyl-2-oxoindolin-5-yl)methyl)piperidin-4-yl)methyl)piperazin-1-yl)-9H-pyrimido[4,5-b]indol-4-yl)-3-fluoro-2-methylphenyl)ethyl)-1,2,4-oxadiazole-3-carboxamide

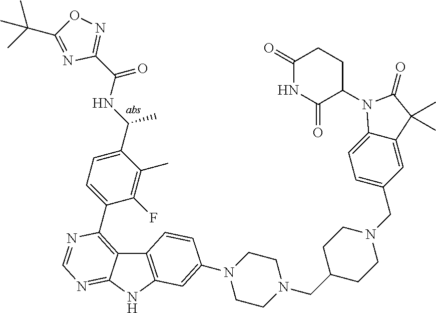 [SMILES: Cc1c([C@@H](C)NC(=O)c2noc(C(C)(C)C)n2)ccc(-c2ncnc3[nH]c4cc(N5CCN(CC6CCN(Cc7ccc8c(c7)C(C)(C)C(=O)N8C7CCC(=O)NC7=O)CC6)CC5)ccc4c23)c1F]

The titled compound was synthesized in the procedures similar to Example 87. ¹H NMR (500 MHz, DMSO) δ 12.18 (s, 1H), 11.06 (s, 1H), 9.59 (d, J=10.0 Hz, 1H), 8.84 (s, 1H), 7.64-7.49 (m, 2H), 7.32-7.07 (m, 3H), 6.95-6.86 (m, 3H), 5.50-5.38 (m, 1H), 5.22 (s, 1H), 3.45 (s, 2H), 3.22 (s, 4H), 2.93-2.80 (m, 3H), 2.68-2.55 (m, 3H), 2.49-2.47 (m, 2H), 2.40 (s, 3H), 2.18 (d, J=5.0 Hz, 2H), 1.96 (t, J=10.0 Hz, 3H), 1.69 (d, J=10.0 Hz, 2H), 1.56 (d, J=10.0 Hz, 3H), 1.54-1.49 (m, 1H), 1.43 (s, 9H), 1.29 (d, J=5.0 Hz, 5H), 1.18-1.08 (m, 2H); [M+H]⁺=938.1.

### Example 99: 5-(tert-butyl)-N-((1R)-1-(4-(7-(4-((1′-(2,6-dioxopiperidin-3-yl)-2′-oxospiro[cyclopropane-1,3′-indolin]-4′-yl)methyl)piperazin-1-yl)-9H-pyrimido[4,5-b]indol-4-yl)-5-fluoro-2-methylphenyl)ethyl)-1,2,4-oxadiazole-3-carboxamide

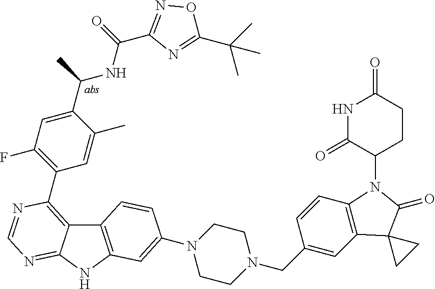 [SMILES: Cc1cc(-c2ncnc3[nH]c4cc(N5CCN(Cc6ccc7c(c6)C6(CC6)C(=O)N7C6CCC(=O)NC6=O)CC5)ccc4c23)c(F)cc1[C@@H](C)NC(=O)c1noc(C(C)(C)C)n1]

The titled compound was synthesized in the procedures similar to Example 89. ¹H NMR (500 MHz, DMSO) δ 12.21 (s, 1H), 11.09 (s, 1H), 9.53 (d, J=10.0 Hz, 1H), 8.85 (d, J=1.5 Hz, 1H), 7.60-7.45 (m, 2H), 7.30-7.13 (m, 2H), 7.08-6.85 (m, 5H), 5.47-5.22 (m, 2H), 3.39 (s, 2H), 3.23 (s, 4H), 2.87 (t, J=150.0 Hz, 1H), 2.71-2.52 (m, 6H), 2.46-2.37 (m, 5H), 1.98 (d, J=10.0 Hz, 1H), 1.56 (d, J=6.5 Hz, 3H), 1.48-1.36 (d, J=1.3 Hz, 11H); [M+H]⁺=839.5.

### Example 100: (R)-3-(tert-butyl)-N-(1-(4-(7-(2-((1-(4-(2,4-dioxotetrahydropyrimidin-1(2H)-yl)phenyl)piperidin-4-yl)methyl)-2,8-diazaspiro[4.5]decan-8-yl)-9H-pyrimido[4,5-b]indol-4-yl)-2-methylphenyl)ethyl)-1,2,4-oxadiazole-5-carboxamide

### Step 1: tert-butyl 8-(4-((2-(trimethylsilyl)ethoxy)methoxy)-9-((2-(trimethylsilyl)ethoxy)methyl)-9H-pyrimido[4,5-b]indol-7-yl)-2,8-diazaspiro[4.5]decane-2-carboxylate

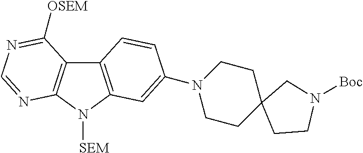

To a stirred mixture of 7-bromo-4-((2-(trimethylsilyl)ethoxy)methoxy)-9-((2-(trimethylsilyl)ethoxy)methyl)-9H-pyrimido[4,5-b]indole (6 g, 11.44 mmol) and tert-butyl 2,8-diazaspiro[4.5]decane-2-carboxylate (3.3 g, 13.72 mmol) in dioxane (120 mL) were added XPhos (818 mg, 1.72 mmol), XPhos Pd G3 (1.45 g, 1.72 mmol) and Cs₂CO₃ (4.47 g, 13.72 mmol) at room temperature under nitrogen atmosphere. The resulting mixture was stirred for 16 h at 100° C. under nitrogen atmosphere. The resulting mixture was filtered, and the filter cake was washed with EtOAc (3×50 mL). The filtrate was concentrated under reduced pressure. The residue was purified by silica gel column chromatography, eluted with 5%-50% EtOAc in petroleum ether to afford the product (4.5 g, 57%). [M+H]⁺=684.4.

### Step 2: 7-(2,8-diazaspiro[4.5]decan-8-yl)-3,9-dihydro-4H-pyrimido[4,5-b]indol-4-one

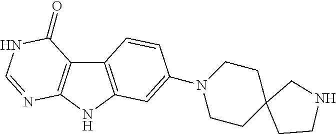 [SMILES: O=c1[nH]cnc2[nH]c3cc(N4CCC5(CCNC5)CC4)ccc3c12]

A solution of tert-butyl 8-(4-((2-(trimethylsilyl)ethoxy)methoxy)-9-((2-(trimethylsilyl)ethoxy)methyl)-9H-pyrimido[4,5-b]indol-7-yl)-2,8-diazaspiro[4.5]decane-2-carboxylate (6.3 g, 9.210 mmol) in TFA (120 mL) was stirred for 2 h at room temperature. The resulting mixture was concentrated under reduced pressure. To the residue were added DCM (70 mL), MeOH (10 mL) and basified to pH 8 with NH₃·H₂O. The resulting mixture was stirred for 24 h at room temperature and concentrated under reduced pressure. The residue was diluted with MTBE (80 mL) and filtered. The filter cake was washed with DCM (3×50 mL). The solid was dried under infrared light. This resulted in the product (8 g, crude). [M+H]⁺=324.2.

### Step 3: benzyl 8-(4-oxo-4,9-dihydro-3H-pyrimido[4,5-b]indol-7-yl)-2,8-diazaspiro[4.5]decane-2-carboxylate

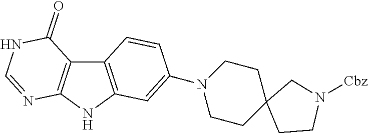 [SMILES: O=C(OCc1ccccc1)N1CCC2(CCN(c3ccc4c(c3)[nH]c3nc[nH]c(=O)c34)CC2)C1]

To a stirred solution of 7-(2,8-diazaspiro[4.5]decan-8-yl)-3,9-dihydro-4H-pyrimido[4,5-b]indol-4-one (4 g, 12.369 mmol) and TEA (3.44 mL, 24.738 mmol) in DCM (80 mL) was added CbzCl (1.48 mL, 8.658 mmol) dropwise at 0° C. under nitrogen atmosphere. The resulting mixture was stirred for 2 h at 0° C. under nitrogen atmosphere. To the above mixture was added CbzCl (1.05 mL, 6.184 mmol, 0.5 equiv) dropwise at 0° C. The resulting mixture was stirred for 16 h at room temperature and concentrated under reduced pressure. The residue was purified by silica gel column chromatography, eluted with 0-10% MeOH in DCM to afford the product (4 g, 70%). [M+H]⁺=458.3.

### Step 4: benzyl 8-(4-chloro-9H-pyrimido[4,5-b]indol-7-yl)-2,8-diazaspiro[4.5]decane-2-carboxylate

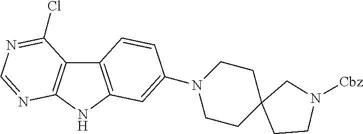 [SMILES: O=C(OCc1ccccc1)N1CCC2(CCN(c3ccc4c(c3)[nH]c3ncnc(Cl)c34)CC2)C1]

A solution of benzyl 8-(4-oxo-4,9-dihydro-3H-pyrimido[4,5-b]indol-7-yl)-2,8-diazaspiro[4.5]decane-2-carboxylate (4.2 g, 9.180 mmol) and POCl₃ (8.56 mL, 91.800 mmol) in dioxane (80 mL) was stirred for 1 h at 90° C. under nitrogen atmosphere. The resulting mixture was concentrated under reduced pressure. The mixture was basified to pH 8 with saturated NaHCO₃ (aq.). The resulting mixture was extracted with EtOAc (3×80 mL). The combined organic layers were washed with brine (2×30 mL) and dried over anhydrous Na₂SO₄. After filtration, the filtrate was concentrated under reduced pressure. This resulted in the product (1.1 g, 25%). [M+H]⁺=476.4.

### Step 5: benzyl (R)-8-(4-(4-(1-(3-(tert-butyl)-1,2,4-oxadiazole-5-carboxamido)ethyl)-3-methylphenyl)-9H-pyrimido[4,5-b]indol-7-yl)-2,8-diazaspiro[4.5]decane-2-carboxylate

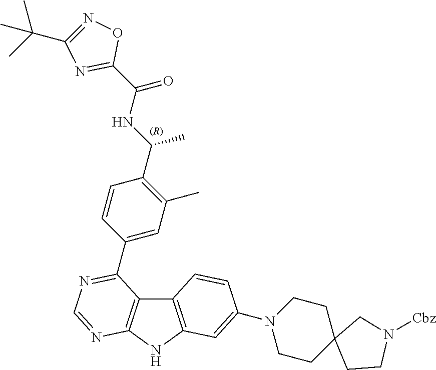

To a stirred mixture of 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (990 mg, 2.40 mmol) and benzyl 8-(4-chloro-9H-pyrimido[4,5-b]indol-7-yl)-2,8-diazaspiro[4.5]decane-2-carboxylate (950 mg, 2.0 mmol) in dioxane (20 mL) and H₂O (4 mL) were added Pd(dppf)Cl₂ (146 mg, 0.200 mmol) and K₃PO₄ (847 mg, 3.992 mmol) at room temperature under nitrogen atmosphere. The resulting mixture was stirred for 16 h at 80° C. under nitrogen atmosphere. The mixture was allowed to cool down to room temperature and diluted with water (100 mL). The resulting mixture was extracted with EtOAc (3×100 mL). The combined organic layers were washed with brine (2×80 mL) and dried over anhydrous Na₂SO₄. After filtration, the filtrate was concentrated under reduced pressure. The residue was purified by silica gel column chromatography, eluted with 0%-10% MeOH in DCM to afford the product (960 mg, 66%). [M+H]⁺=727.3.

### Step 6: (R)—N-(1-(4-(7-(2,8-diazaspiro[4.5]decan-8-yl)-9H-pyrimido[4,5-b]indol-4-yl)-2-methylphenyl)ethyl)-3-(tert-butyl)-1,2,4-oxadiazole-5-carboxamide formate

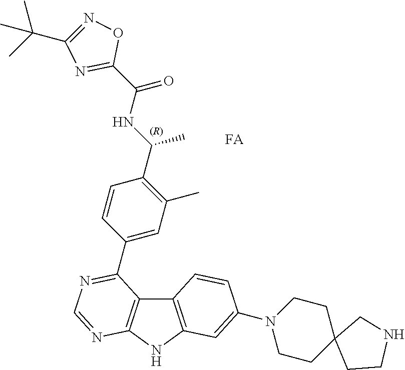

A solution of benzyl (R)-8-(4-(4-(1-(3-(tert-butyl)-1,2,4-oxadiazole-5-carboxamido)ethyl)-3-methylphenyl)-9H-pyrimido[4,5-b]indol-7-yl)-2,8-diazaspiro[4.5]decane-2-carboxylate (950 mg, 1.31 mmol) in TFA (40 mL) was stirred for 16 h at 70° C. under nitrogen atmosphere. The resulting mixture was concentrated under reduced pressure. The residue was purified by reverse phase flash chromatography with the following conditions: column, C18, MeCN in water (0.1% FA), 5% to 40% gradient in 20 min; Detector, UV 254 nm. This afforded the product (641 mg, 76%). ¹H NMR (400 MHz, DMSO) δ 12.19 (s, 1H), 9.97 (d, J=8.0 Hz, 1H), 8.82 (s, 1H), 8.24 (s, 1H), 7.82-7.65 (m, 4H), 6.97-6.92 (m, 2H), 5.46-5.38 (m, 1H), 3.30 (s, 4H), 3.28-3.21 (m, 2H), 3.02 (s, 2H), 2.50 (s, 3H), 1.83 (t, J=7.6 Hz, 2H), 1.69 (s, 4H), 1.58 (d, J=7.2 Hz, 3H), 1.38 (s, 9H); [M+H]⁺=593.6.

### Step 7: (R)-3-(tert-butyl)-N-(1-(4-(7-(2-((1-(4-(2,4-dioxotetrahydropyrimidin-1(2H)-yl)phenyl)piperidin-4-yl)methyl)-2,8-diazaspiro[4.5]decan-8-yl)-9H-pyrimido[4,5-b]indol-4-yl)-2-methylphenyl)ethyl)-1,2,4-oxadiazole-5-carboxamide

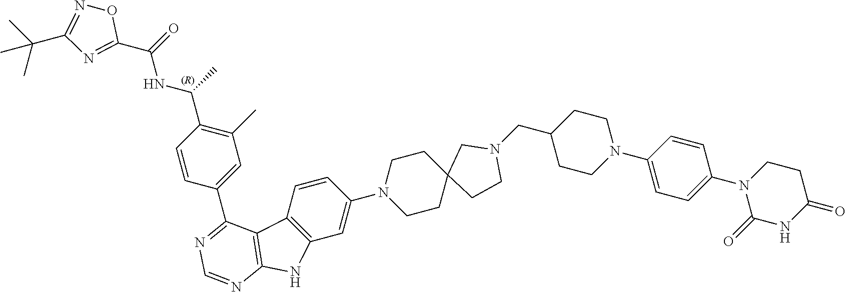

A mixture of (R)—N-(1-(4-(7-(2,8-diazaspiro[4.5]decan-8-yl)-9H-pyrimido[4,5-b]indol-4-yl)-2-methylphenyl)ethyl)-3-(tert-butyl)-1,2,4-oxadiazole-5-carboxamide formate (0.15 g, 0.253 mmol) and 1-(4-(2,4-dioxotetrahydropyrimidin-1(2H)-yl)phenyl)piperidine-4-carbaldehyde (0.095 g, 0.315 mmol) in dichloromethane (8 mL) and MeOH (1.60 mL) was stirred in a round bottom flask at room temperature for 0.5 hour. To the mixture was added NaBH(OAc)₃ (0.081 g, 0.382 mmol) and stirred at room temperature for 1 hour. Then the mixture was evaporated in vacuum to afford the crude product, which was purified by pre-HPLC to afford the product (0.11 g, 50%). ¹H NMR (400 MHz, DMSO) δ 12.12 (s, 1H), 10.25 (s, 1H), 9.93 (d, J=7.9 Hz, 1H), 8.81 (d, J=0.7 Hz, 1H), 7.80-7.65 (m, 4H), 7.12 (d, J=8.6 Hz, 2H), 6.92 (d, J=9.0 Hz, 4H), 5.42 (t, J=7.3 Hz, 1H), 3.61-3.73 (m, 4H), 3.17-3.35 (m, 4H), 2.70-2.61 (m, 4H), 2.50-2.58 (m, 5H), 2.37 (s, 2H), 2.27 (d, J=6.9 Hz, 2H), 1.80 (d, J=11.4 Hz, 2H), 1.67-1.56 (m, 6H), 1.37 (s, 9H), 1.10-1.25 (m, 6H); [M+H]⁺=878.7.

### Example 101: 3-(tert-butyl)-N—((R)-1-(4-(7-(2-(3-(4-((R)-2,6-dioxopiperidin-3-yl)phenyl)cyclobutyl)-2,8-diazaspiro[4.5]decan-8-yl)-9H-pyrimido[4,5-b]indol-4-yl)-2-methylphenyl)ethyl)-1,2,4-oxadiazole-5-carboxamide

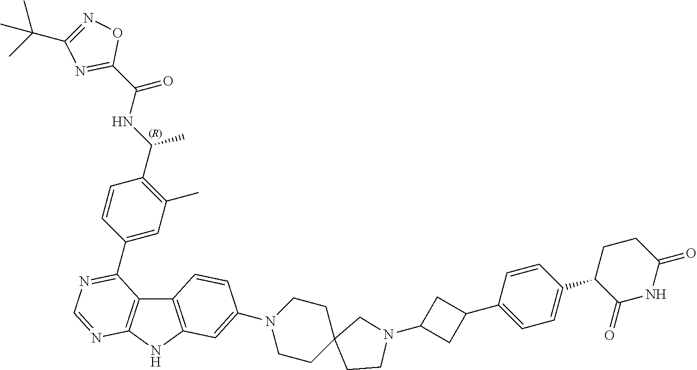

The titled compound was synthesized in the procedures similar to Example 100. ¹H NMR (400 MHz, DMSO) δ 12.14 (s, 1H), 10.82 (s, 1H), 9.93 (d, J=7.8 Hz, 1H), 8.81 (d, J=1.9 Hz, 1H), 8.16 (s, 1H), 7.79-7.64 (m, 4H), 7.27-7.12 (m, 4H), 6.92 (d, J=8.6 Hz, 2H), 5.47-5.38 (m, 1H), 3.82 (dd, J=11.4, 4.8 Hz, 1H), 3.21-3.65 (m, 4H), 2.52-2.85 (m, 5H), 2.54 (d, J=2.0 Hz, 2H), 2.47-2.39 (m, 3H), 2.10-2.25 (m, 2H), 1.95-2.05 (m, 3H), 1.76-1.53 (m, 10H), 1.33 (s, 9H); [M+H]⁺=834.7.

### Example 102: 3-(tert-butyl)-N—((R)-1-(4-(7-(2-(3-(4-((R)-2,6-dioxopiperidin-3-yl)phenyl)cyclobutyl)-2,8-diazaspiro[4.5]decan-8-yl)-9H-pyrimido[4,5-b]indol-4-yl)-2-methylphenyl)ethyl)-1,2,4-oxadiazole-5-carboxamide

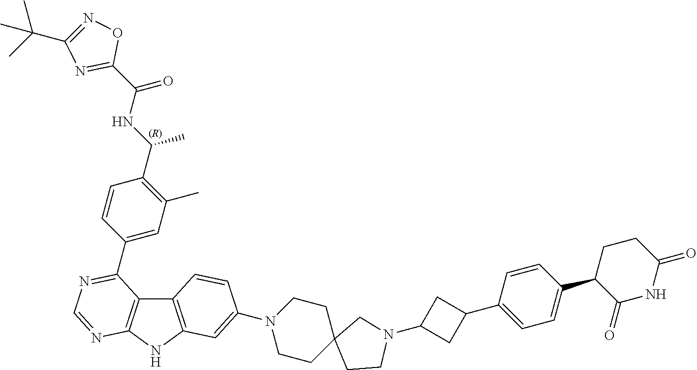

The titled compound was synthesized in the procedures similar to Example 100. ¹H NMR (400 MHz, DMSO) δ 12.13 (s, 1H), 10.82 (s, 1H), 9.93 (d, J=7.9 Hz, 1H), 8.81 (s, 1H), 7.79-7.65 (m, 4H), 7.18 (dd, J=27.7, 8.2 Hz, 4H), 6.92 (d, J=8.4 Hz, 2H), 5.46-5.37 (m, 1H), 3.82 (dd, J=11.5, 4.9 Hz, 1H), 3.05-3.30 (m, 6H), 2.53-2.79 (m, 5H), 2.50 (s, 3H), 2.48-2.36 (m, 3H), 2.10-2.25 (m, 1H), 1.95-2.05 (m, 3H), 1.61-1.74 (m, 6H), 1.58 (d, J=7.0 Hz, 3H), 1.37 (s, 9H); [M+H]⁺=834.6.

### Example 103: 3-(tert-butyl)-N-((1R)-1-(4-(7-(2-(3-(4-((2,6-dioxopiperidin-3-yl)amino)phenyl)cyclobutyl)-2,8-diazaspiro[4.5]decan-8-yl)-9H-pyrimido[4,5-b]indol-4-yl)-2-methylphenyl)ethyl)-1,2,4-oxadiazole-5-carboxamide

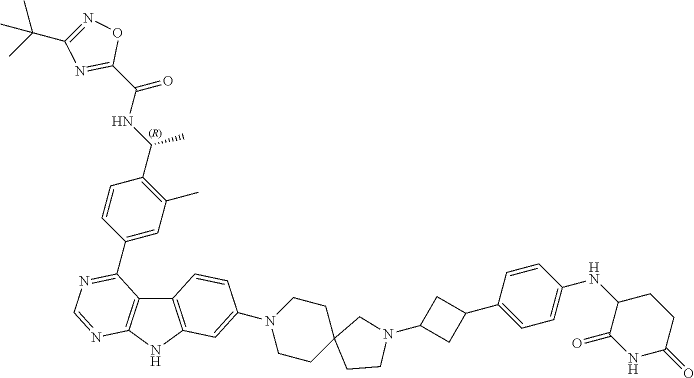

The titled compound was synthesized in the procedures similar to Example 100. ¹H NMR (400 MHz, DMSO) δ 12.12 (s, 1H), 10.76 (s, 1H), 9.93 (d, J=7.9 Hz, 1H), 8.81 (s, 1H), 7.81-7.63 (m, 4H), 6.96 (dt, J=20.0, 8.4 Hz, 4H), 6.64-6.57 (m, 2H), 5.66 (d, J=7.5 Hz, 1H), 5.44-5.38 (m, 1H), 4.27 (dd, J=15.4, 8.3 Hz, 1H), 3.28-3.19 (m, 4H), 3.00-2.81 (m, 2H), 2.78-2.69 (m, 1H), 2.65-2.53 (m, 3H), 2.41-2.25 (m, 7H), 2.15-2.08 (m, 2H), 1.91-1.80 (m, 2H), 1.71-1.55 (m, 10H), 1.37 (s, 9H); [M+H]⁺=849.6.

### Example 104: (R)-3-(tert-butyl)-N-(1-(4-(7-((4-((1-(4-(2,4-dioxotetrahydropyrimidin-1(2H)-yl)phenyl)piperidin-4-yl)methyl)piperazin-1-yl)methyl)-9H-pyrimido[4,5-b]indol-4-yl)-2-methylphenyl)ethyl)-1,2,4-oxadiazole-5-carboxamide

### Step 1: 7-bromo-4-chloro-9H-pyrimido[4,5-b]indole

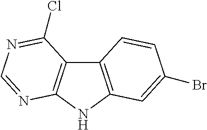 [SMILES: Clc1ncnc2[nH]c3cc(Br)ccc3c12]

To a stirred solution of 7-bromo-3,9-dihydro-4H-pyrimido[4,5-b]indol-4-one (10 g, 37.87 mmol) in dioxane (100 mL) was added POCl₃ (100 mL) dropwise at room temperature under nitrogen. The resulting mixture was stirred for 2 h at 80° C. under nitrogen atmosphere and concentrated under reduced pressure. The mixture was neutralized to PH=7 with sat. NaHCO₃ (aq.). The precipitated solid was collected by filtration and washed with EtOAc (3×100 mL). The resulting solid was dried under infrared lamp to give the product (4 g, 38%). [M+H]⁺=281.9.

### Step 2: 4-chloro-7-vinyl-9H-pyrimido[4,5-b]indole

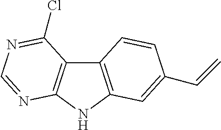 [SMILES: C=Cc1ccc2c(c1)[nH]c1ncnc(Cl)c12]

To a solution of 7-bromo-4-chloro-9H-pyrimido[4,5-b]indole (3 g, 10.618 mmol) and potassium vinyltrifluoroborate (1.71 g, 12.742 mmol) in dioxane (60 mL) and water (12 mL) were added Cs₂CO₃ (6.92 g, 21.236 mmol) and Pd(dppf)Cl₂ (0.78 g, 1.062 mmol). After stirring for 16 h at 80° C. under nitrogen atmosphere, the desired product was detected by LCMS. The resulting mixture was diluted with water (200 mL) and filtered. The filter cake was washed with DCM (3×100 mL). The filtrate was concentrated under reduced pressure to afford the product (2.2 g, crude), which was used in the next step directly without further purification. [M+H]⁺=230.0.

### Step 3: tert-butyl 4-chloro-7-vinyl-9H-pyrimido[4,5-b]indole-9-carboxylate

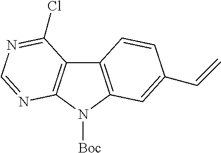 [SMILES: C=Cc1ccc2c3c(Cl)ncnc3n(C(=O)OC(C)(C)C)c2c1]

To a stirred solution of 4-chloro-7-vinyl-9H-pyrimido[4,5-b]indole (2.2 g, 9.579 mmol), DIEA (2.48 g, 19.158 mmol) and DMAP (0.12 g, 0.958 mmol) in DMF (30 mL) was added Boc₂O (2.51 g, 11.495 mmol) at 0° C. under nitrogen atmosphere. The resulting mixture was stirred overnight at room temperature under nitrogen atmosphere and diluted with water (200 mL). The resulting mixture was extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 1-30% EtOAc in PE to afford the product (3 g, 95%). [M+H]⁺=330.1.

### Step 4: tert-butyl 4-chloro-7-formvl-9H-pyrimido[4,5-b]indole-9-carboxylate

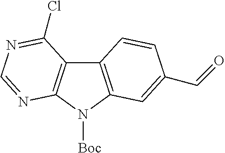 [SMILES: CC(C)(C)OC(=O)n1c2cc(C=O)ccc2c2c(Cl)ncnc21]

To a stirred solution of tert-butyl 4-chloro-7-vinyl-9H-pyrimido[4,5-b]indole-9-carboxylate (2.2 g, 6.671 mmol) and NaIO₄ (5.71 g, 26.684 mmol) in dioxane (40 mL) and water (10 mL) was added K₂OsO₄·2H₂O (0.25 g, 0.667 mmol) at room temperature under nitrogen atmosphere. After stirring for 2 h at room temperature, the resulting mixture was extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 1-30% EtOAc in PE to afford the product (1.6 g, 73%). [M+H]⁺=332.0.

### Step 5: (R)-3-(tert-butyl)-N-(1-(4-(7-formyl-9H-pyrimido[4,5-b]indol-4-yl)-2-methylphenyl)ethyl)-1,2,4-oxadiazole-5-carboxamide

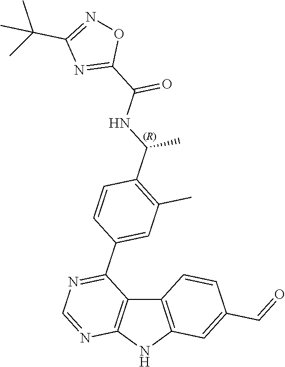

To a solution of tert-butyl 4-chloro-7-formyl-9H-pyrimido[4,5-b]indole-9-carboxylate (1.2 g, 3.62 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (1.79 g, 4.34 mmol) in DMF (60 mL) and water (12 mL) were added K₃PO₄ (1.54 g, 7.234 mmol) and Pd(dppf)Cl₂ (0.26 g, 0.362 mmol). After stirring overnight at 80° C. under nitrogen atmosphere, the resulting mixture was concentrated under reduced pressure. The residue was purified by reverse phase flash chromatography with the following conditions: column, C18; mobile phase, ACN in water (0.1% FA), 10% to 50% gradient in 10 min; detector, UV 254 nm. This afford the product (192 mg, 11%). [M+H]⁺=483.3.

### Step 6: tert-butyl (R)-4-((4-(4-(1-(3-(tert-butyl)-1,2,4-oxadiazole-5-carboxamido)ethyl)-3-methylphenyl)-9H-pyrimido[4,5-b]indol-7-yl)methyl)piperazine-1-carboxylate

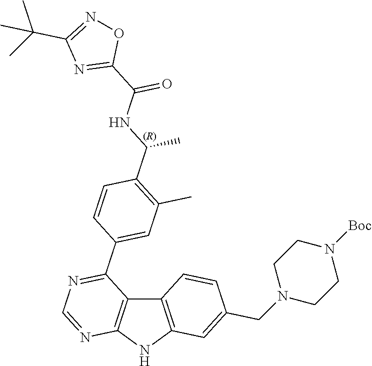

To a solution of tert-butyl piperazine-1-carboxylate (63 mg, 0.342 mmol) in dichloromethane (4 mL) and MeOH (4 mL), (R)-3-(tert-butyl)-N-(1-(4-(7-formyl-9H-pyrimido[4,5-b]indol-4-yl)-2-methylphenyl)ethyl)-1,2,4-oxadiazole-5-carboxamide (110 mg, 0.228 mmol) and HOAc (0.06 mL) were added. The mixture was stirred at room temperature overnight. Then NaBH(OAc)₃ (241 mg, 1.14 mmol) was added and stirred at room temperature for 2 hours. The reaction mixture was concentrated in vacuum, and the residue was purified by silica gel column chromatography (DCM:MeOH=100%: 0%˜90%: 10% gradient elution) to afford desired product (125 mg, 84%). [M+H]⁺=653.5.

### Step 7: (R)-3-(tert-butyl)-N-(1-(2-methyl-4-(7-(piperazin-1-ylmethyl)-9H-pyrimido[4,5-b]indol-4-yl)phenyl)ethyl)-1,2,4-oxadiazole-5-carboxamide hydrochloride

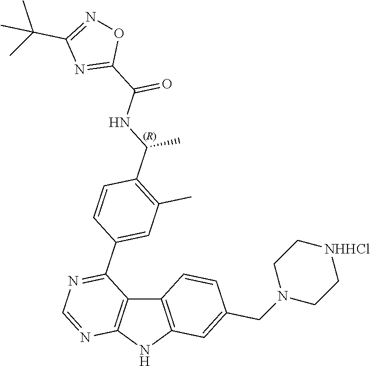

To a solution of tert-butyl (R)-4-((4-(4-(1-(3-(tert-butyl)-1,2,4-oxadiazole-5-carboxamido)ethyl)-3-methylphenyl)-9H-pyrimido[4,5-b]indol-7-yl)methyl)piperazine-1-carboxylate (125 mg, 0.192 mmol) in DCM (2 mL), HCl/dioxane (4M, 10 mL) was added. The mixture was stirred at room temperature overnight. Then the mixture was concentrated in vacuo to give the product (283 mg, crude), which was used in next step without further purification. [M+H]⁺=553.4.

### Step 8: (R)-3-(tert-butyl)-N-(1-(4-(7-((4-((1-(4-(2,4-dioxotetrahydropyrimidin-1(2H)-yl)phenyl)piperidin-4-yl)methyl)piperazin-1-yl)methyl)-9H-pyrimido[4,5-b]indol-4-yl)-2-methylphenyl)ethyl)-1,2,4-oxadiazole-5-carboxamide

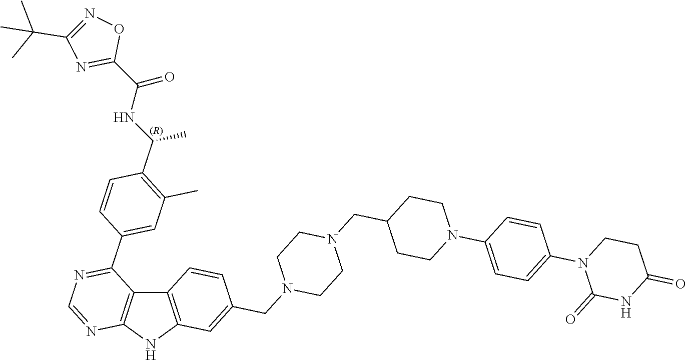

A mixture of (R)-3-(tert-butyl)-N-(1-(2-methyl-4-(7-(piperazin-1-ylmethyl)-9H-pyrimido[4,5-b]indol-4-yl)phenyl)ethyl)-1,2,4-oxadiazole-5-carboxamide hydrochloride (100 mg, crude), 1-(4-(2,4-dioxotetrahydropyrimidin-1(2H)-yl)phenyl)piperidine-4-carbaldehyde (65 mg, 0.217 mmol) and NaOAc (44 mg, 0.543 mmol) in dichloromethane (4 mL) and MeOH (4 mL) was stirred at room temperature for 5 mins. Then HOAc (0.06 mL) was added. The mixture was stirred at room temperature overnight. Then NaBH(OAc)₃ (191 mg, 0.905 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the product, which was further purified by prep-TLC (DCM:MeOH=10:1) to afford desired product (63.8 mg, 42%). ¹H NMR (500 MHz, DMSO) δ 12.42 (s, 1H), 10.25 (s, 1H), 9.94 (d, J=8.0 Hz, 1H), 8.94 (s, 1H), 7.92-7.66 (m, 4H), 7.53 (s, 1H), 7.20-7.07 (m, 3H), 6.91 (d, J=8.0 Hz, 2H), 5.58-5.31 (m, 1H), 3.84-3.55 (m, 6H), 2.71-2.58 (m, 5H), 2.55-2.49 (m, 3H), 2.48-2.28 (m, 6H), 2.15 (s, 2H), 1.91 (s, 2H), 1.76 (d, J=8.0 Hz, 2H), 1.59-1.56 (m, 4H), 1.37 (s, 9H), 1.25-1.12 (m, 2H); [M+H]⁺=838.7.

### Example 105: (R)-3-(tert-butyl)-N-(1-(4-(7-(2-(4-(1-(4-(2,4-dioxotetrahydropyrimidin-1(2H)-yl)phenyl)azetidin-3-yl)piperazin-1-yl)ethyl)-9H-pyrimido[4,5-b]indol-4-yl)-2-methylphenyl)ethyl)-1,2,4-oxadiazole-5-carboxamide

### Step 1: (E)-4-chloro-7-(2-ethoxyvinvl)-9H-pyrimido[4,5-b]indole

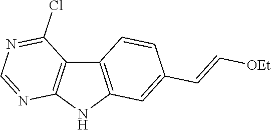 [SMILES: CCO/C=C/c1ccc2c(c1)[nH]c1ncnc(Cl)c12]

To a stirred solution of 7-bromo-4-chloro-9H-pyrimido[4,5-b]indole (7 g, 21.30 mmol) and 2-[(E)-2-ethoxyethenyl]-4,4,5,5-tetramethyl-1,3,2-dioxaborolane (5.06 g, 25.56 mmol) in dioxane (100 mL) and water (20 mL) were added Pd₂(dba)₃ (0.98 g, 1.07 mmol), PCy₃ (0.60 g, 2.13 mmol) and K₂CO₃ (5.88 g, 42.60 mmol) at room temperature under nitrogen. The resulting mixture was stirred for 2 h at 80° C. under nitrogen atmosphere and concentrated under reduced pressure. The residue was purified by silica gel column chromatography, eluted with 1-50% EtOAc in CH₂Cl₂ to afford the product (1.6 g, 28%). [M+H]⁺=274.0.

### Step 2: (R,E)-3-(tert-butyl)-N-(1-(4-(7-(2-ethoxyvinyl)-9H-pyrimido[4,5-b]indol-4-yl)-2-methylphenyl)ethyl)-1,2,4-oxadiazole-5-carboxamide

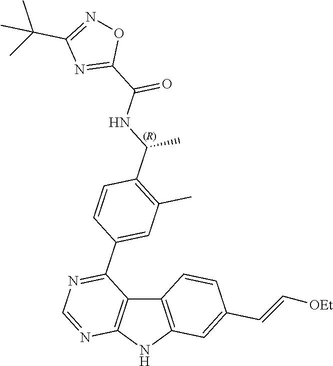

To a solution of (E)-4-chloro-7-(2-ethoxyvinyl)-9H-pyrimido[4,5-b]indole (1.5 g, 5.48 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (the compound was obtained through the same way in WO2021219070A) (2.72 g, 6.58 mmol) in DMF (75 mL) and water (15 mL) were added K₃PO₄ (2.33 g, 10.96 mmol) and Pd(dppf)Cl₂ (0.40 g, 0.55 mmol). After stirring for 2 h at 80° C. under nitrogen atmosphere, the resulting mixture was diluted with water (200 mL) and extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography, eluted with 1-50% EtOAc in CH₂Cl₂ to afford the product (934 mg, 33%). ¹H NMR (400 MHz, DMSO) δ 12.36 (s, 1H), 9.94 (d, J=8.0 Hz, 1H), 8.90 (s, 1H), 7.80-7.71 (m, 5H), 7.40-7.32 (m, 2H), 7.23-7.20 (d, J=12.8 Hz, 1H), 6.01 (d, J=12.8 Hz, 1H), 5.43 (t, J=6.8 Hz, 1H), 3.94 (q, J=7.2 Hz, 2H), 1.59 (d, J=6.8 Hz, 3H), 1.38 (s, 9H), 1.28 (t, J=7.2 Hz, 3H); [M+H]⁺=525.3.

### Step 3: (R)-3-(tert-butyl)-N-(1-(2-methyl-4-(7-(2-oxoethyl)-9H-pyrimido[4,5-b]indol-4-yl)phenyl)ethyl)-1,2,4-oxadiazole-5-carboxamide

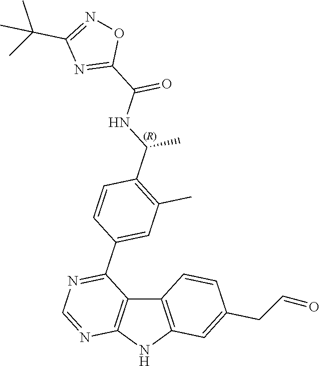

A mixture of (R,E)-3-(tert-butyl)-N-(1-(4-(7-(2-ethoxyvinyl)-9H-pyrimido[4,5-b]indol-4-yl)-2-methylphenyl)ethyl)-1,2,4-oxadiazole-5-carboxamide (934 mg, 1.87 mmol) and HCOOH (10 mL) was stirred at room temperature for 2 h. Then the mixture was concentrated in vacuo to give crude product, which was used in next step without further purification. [M+H]⁺=497.3.

### Step 4: tert-butyl (R)-4-(2-(4-(4-(1-(3-(tert-butyl)-1,2,4-oxadiazole-5-carboxamido)ethyl)-3-methylphenyl)-9H-pyrimido[4,5-b]indol-7-yl)ethyl)piperazine-1-carboxylate

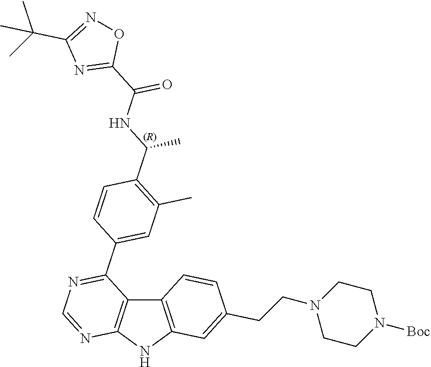

To a solution of tert-butyl piperazine-1-carboxylate (100 mg, 0.539 mmol) in dichloromethane (4 mL) and MeOH (4 mL), (R)-3-(tert-butyl)-N-(1-(2-methyl-4-(7-(2-oxoethyl)-9H-pyrimido[4,5-b]indol-4-yl)phenyl)ethyl)-1,2,4-oxadiazole-5-carboxamide (223 mg, crude), NaOAc (110 mg, 1.347 mmol) and HOAc (0.06 mL) were added. The mixture was stirred at room temperature overnight. Then NaBH(OAc)₃ (476 mg, 2.25 mmol) was added and stirred at room temperature for 2 hours. The reaction mixture was concentrated in vacuum, and the residue was purified by silica gel column chromatography (DCM:MeOH=100%: 0%˜90%: 10% gradient elution) to afford desired product (90 mg, 30%). [M+H]⁺=667.5.

### Step 5: (R)-3-(tert-butyl)-N-(1-(2-methyl-4-(7-(2-(piperazin-1-yl)ethyl)-9H-pyrimido[4,5-b]indol-4-yl)phenyl)ethyl)-1,2,4-oxadiazole-5-carboxamide hydrochloride

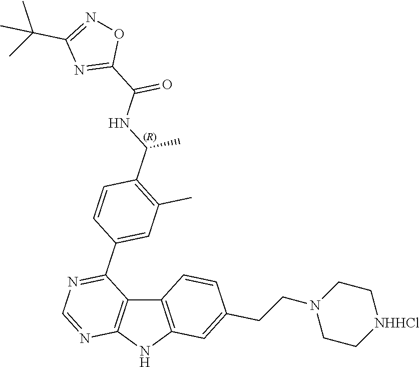

A mixture of tert-butyl (R)-4-(2-(4-(4-(1-(3-(tert-butyl)-1,2,4-oxadiazole-5-carboxamido)ethyl)-3-methylphenyl)-9H-pyrimido[4,5-b]indol-7-yl)ethyl)piperazine-1-carboxylate (90 mg, 0.135 mmol) in HCl/dioxane (4 M, 10 mL) was stirred at room temperature for 4 h. Then the mixture was concentrated in vacuo to give crude product (90 mg, crude), which was used in next step without further purification.

### Step 6: (R)-3-(tert-butyl)-N-(1-(4-(7-(2-(4-(1-(4-(2,4-dioxo-3-((2-(trimethylsilyl)ethoxy)methyl)tetrahydropyrimidin-1(2H)-yl)phenyl)azetidin-3-yl)piperazin-1-yl)ethyl)-9H-pyrimido[4,5-b]indol-4-yl)-2-methylphenyl)ethyl)-1,2,4-oxadiazole-5-carboxamide

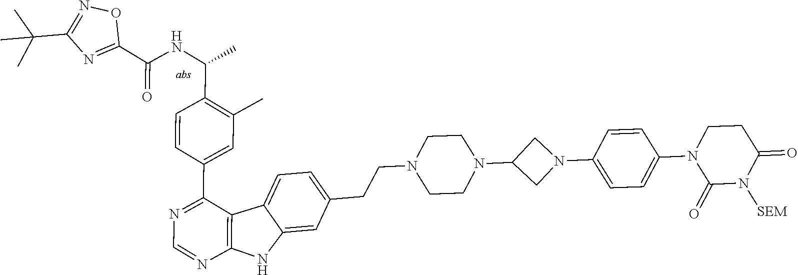 [SMILES: Cc1cc(-c2ncnc3[nH]c4cc(CCN5CCN(C6CN(c7ccc(N8CCC(=O)N(COCC[Si](C)(C)C)C8=O)cc7)C6)CC5)ccc4c23)ccc1[C@@H](C)NC(=O)c1nc(C(C)(C)C)no1]

A mixture of (R)-3-(tert-butyl)-N-(1-(2-methyl-4-(7-(2-(piperazin-1-yl)ethyl)-9H-pyrimido[4,5-b]indol-4-yl)phenyl)ethyl)-1,2,4-oxadiazole-5-carboxamide hydrochloride (90 mg, crude), 1-(4-(2,4-dioxotetrahydropyrimidin-1(2H)-yl)phenyl)piperidine-4-carbaldehyde (61 mg, 0.159 mmol) and NaOAc (39 mg, 0.477 mmol) in dichloromethane (4 mL) and MeOH (4 mL) was stirred at room temperature for 5 mins. Then HOAc (0.06 mL) was added. The mixture was stirred at room temperature overnight. Then NaBH(OAc)₃ (160 mg, 0.759 mmol) was added and stirred at room temperature for 2 hours. The reaction mixture was concentrated in vacuum, and the residue was purified by silica gel column chromatography (DCM:MeOH=100%: 0% 90%: 10% gradient elution) to give crude product, which was further purified by prep-TLC (DCM:MeOH=10:1) to afford desired product (10 mg, 15%). [M+H]⁺=940.7.

### Step 7: (R)-3-(tert-butyl)-N-(1-(4-(7-(2-(4-(1-(4-(2,4-dioxotetrahydropyrimidin-1(2H)-yl)phenyl)azetidin-3-yl)piperazin-1-yl)ethyl)-9H-pyrimido[4,5-b]indol-4-yl)-2-methylphenyl)ethyl)-1,2,4-oxadiazole-5-carboxamide

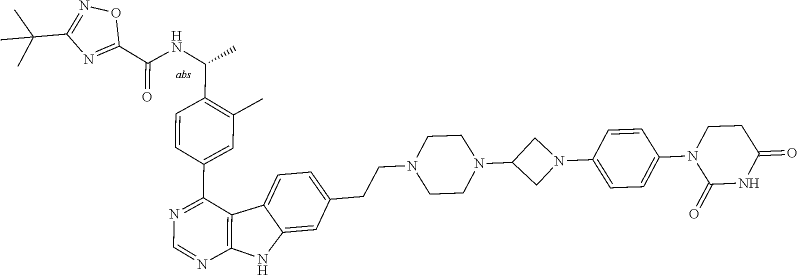 [SMILES: Cc1cc(-c2ncnc3[nH]c4cc(CCN5CCN(C6CN(c7ccc(N8CCC(=O)NC8=O)cc7)C6)CC5)ccc4c23)ccc1[C@@H](C)NC(=O)c1nc(C(C)(C)C)no1]

To a solution of (R)-3-(tert-butyl)-N-(1-(4-(7-(2-(4-(1-(4-(2,4-dioxo-3-((2-(trimethylsilyl)ethoxy)methyl)tetrahydropyrimidin-1(2H)-yl)phenyl)azetidin-3-yl)piperazin-1-yl)ethyl)-9H-pyrimido[4,5-b]indol-4-yl)-2-methylphenyl)ethyl)-1,2,4-oxadiazole-5-carboxamide (10 mg, 0.010 mmol) in dichloromethane (5 mL), TFA was added. The mixture was stirred at room temperature overnight. The reaction mixture was concentrated in vacuum, and the residue was diluted with MeOH (5 mL). NH₃ (7M in MeOH) was added dropwise, and the mixture was stirred at room temperature for 1.5 h. Then the mixture was concentrated in vacuo and the residue was purified by prep-TLC (DCM:MeOH=10:1) to afford desired product (3.6 mg, 41%). ¹H NMR (500 MHz, DMSO) δ 12.40 (s, 1H), 10.23 (s, 1H), 9.93 (d, J=8.0 Hz, 1H), 8.92 (s, 1H), 7.88-7.66 (m, 4H), 7.43 (s, 1H), 7.13-7.07 (m, 3H), 6.42 (d, J=8.0 Hz, 2H), 5.43 (s, 1H), 3.90 (s, 2H), 3.69-3.63 (m, 2H), 3.57 (s, 2H), 3.29 (s, 4H), 2.89 (s, 2H), 2.69-2.64 (m, 2H), 2.58 (s, 2H), 2.36 (s, 2H), 2.18 (s, 1H), 1.58 (d, J=8.0 Hz, 3H), 1.37 (s, 9H), 1.28-1.18 (m, 5H); [M+H]⁺=810.5.

### Example 106: (R)-3-(tert-butyl)-N-(1-(4-(7-(2-(4-(4-(2,4-dioxotetrahydropyrimidin-1(2H)-yl)phenyl)piperazin-1-yl)ethyl)-9H-pyrimido[4,5-b]indol-4-yl)-2-methylphenyl)ethyl)-1,2,4-oxadiazole-5-carboxamide

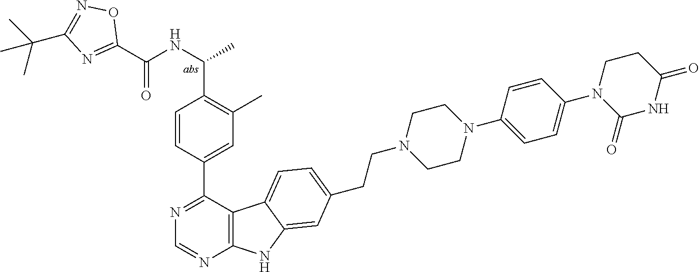 [SMILES: Cc1cc(-c2ncnc3[nH]c4cc(CCN5CCN(c6ccc(N7CCC(=O)NC7=O)cc6)CC5)ccc4c23)ccc1[C@@H](C)NC(=O)c1nc(C(C)(C)C)no1]

The titled compound was synthesized in the procedures similar to Example 105. ¹H NMR (500 MHz, DMSO) δ 12.40 (s, 1H), 10.26 (s, 1H), 9.93 (d, J=8.0 Hz, 1H), 8.93 (s, 1H), 7.85-7.67 (m, 4H), 7.46 (s, 1H), 7.17-7.10 (m, 3H), 6.94 (d, J=8.0 Hz, 2H), 5.58-5.35 (m, 1H), 3.70 (t, J=8.0 Hz, 2H), 3.15 (s, 4H), 2.99-2.91 (m, 2H), 2.71-2.59 (m, 8H), 2.59-2.52 (m, 2H), 1.59 (d, J=8.0 Hz, 3H), 1.37 (s, 9H); [M+H]⁺=755.6.

### Example 107, 108 and 109: 3-(tert-butyl)-N—((R)-1-(4-(7-(9-((1S,3r)-3-(4-(((S)-2,6-dioxopiperidin-3-yl)amino)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide, 3-(tert-butyl)-N-((1R)-1-(4-(7-(9-((1s,3s)-3-(4-((2,6-dioxopiperidin-3-yl)amino)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide and 3-(tert-butyl)-N—((R)-1-(4-(7-(9-((1R,3r)-3-(4-(((R)-2,6-dioxopiperidin-3-yl)amino)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide

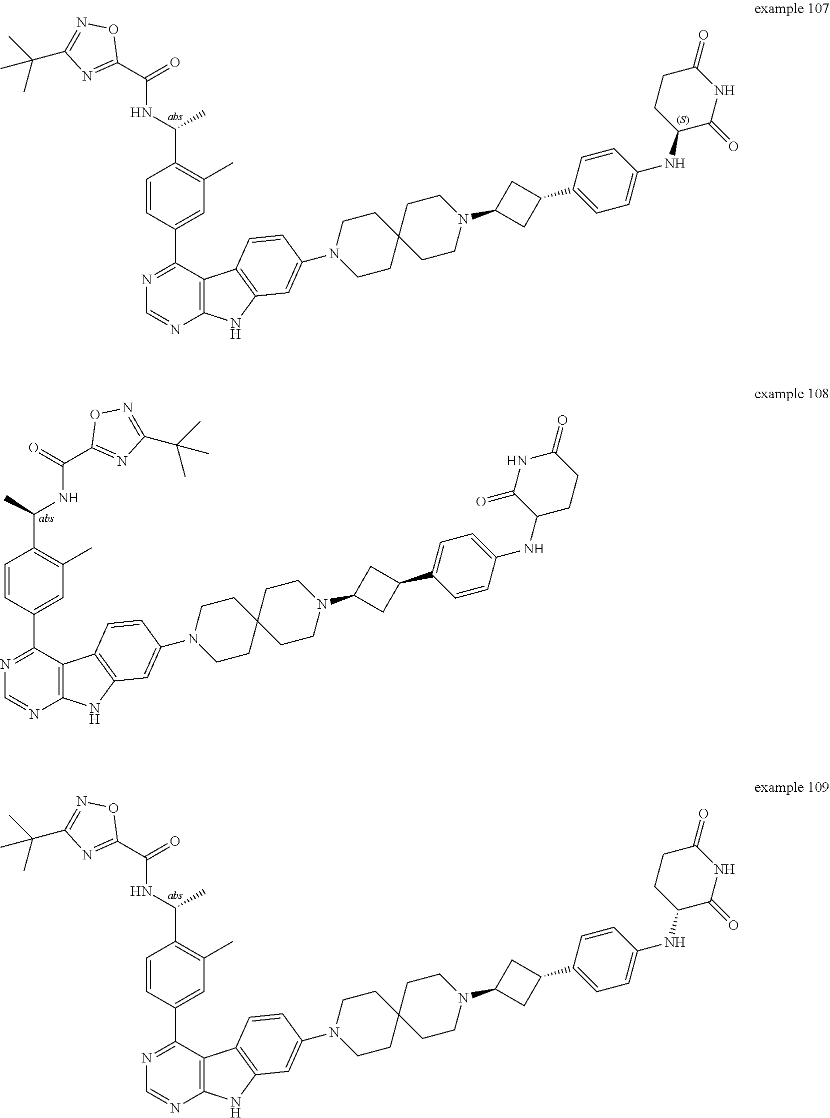

A mixture of (R)—N-(1-(4-(7-(3,9-diazaspiro[5.5]undecan-3-yl)-9H-pyrimido[4,5-b]indol-4-yl)-2-methylphenyl)ethyl)-3-(tert-butyl)-1,2,4-oxadiazole-5-carboxamide (0.08 g, 0.132 mmol) and 3-((4-(3-oxocyclobutyl)phenyl)amino)piperidine-2,6-dione (0.054 g, 0.198 mmol) in 1,2-dichloroethane (4 mL) and HOAc (8 mg) was stirred in a round bottom flask at room temperature for 0.5 hour. To the mixture was added NaBH(OAc)₃ (0.070 g, 0.33 mmol) and stirred at room temperature for 12 hours. Then the mixture was evaporated in vacuum to afford the crude product, which was purified by pre-HPLC to afford example 107 (6.94 mg, 6%), example 108 (38.92 mg, 35%) and example 109 (6.9 mg, 6%). Example 107: ¹H NMR (500 MHz, DMSO) δ 12.12 (s, 1H), 10.77 (s, 1H), 9.93 (d, J=7.8 Hz, 1H), 8.80 (s, 1H), 8.22 (s, 1H), 7.81-7.64 (m, 4H), 7.01 (d, J=8.5 Hz, 2H), 6.91 (d, J=8.5 Hz, 2H), 6.63 (d, J=8.5 Hz, 2H), 5.67 (d, J=7.4 Hz, 1H), 5.46-5.39 (m, 1H), 4.32-4.24 (m, 1H), 3.31-3.21 (m, 6H), 2.93-2.85 (m, 1H), 2.78-2.71 (m, 1H), 2.66-2.55 (m, 1H), 2.50 (s, 3H), 2.32-2.20 (m, 6H), 2.14-2.02 (m, 3H), 1.89-1.82 (m, 1H), 1.61-1.45 (m, 11H), 1.37 (s, 9H); [M+H]⁺=863.6. Example 108: ¹H NMR (500 MHz, DMSO) δ 12.12 (s, 1H), 10.76 (s, 1H), 9.93 (d, J=7.9 Hz, 1H), 8.80 (s, 1H), 8.21 (s, 1H), 7.81-7.62 (m, 4H), 6.93 (dd, J=21.6, 8.5 Hz, 4H), 6.61 (d, J=8.5 Hz, 2H), 5.67 (d, J=7.6 Hz, 1H), 5.46-5.38 (m, 1H), 4.31-4.25 (m, 1H), 3.31-3.20 (m, 5H), 3.01-2.91 (m, 1H), 2.78-2.62 (m, 2H), 2.60-2.52 (m, 1H), 2.50 (s, 3H), 2.41-2.27 (m, 6H), 2.13-2.05 (m, 1H), 1.89-1.71 (m, 3H), 1.60-1.52 (m, 7H), 1.50 (s, 4H), 1.37 (s, 9H); [M+H]⁺=863.6. Example 109: ¹H NMR (500 MHz, DMSO) δ 12.21 (s, 1H), 10.76 (d, J=12.0 Hz, 1H), 9.94 (d, J=7.8 Hz, 1H), 9.11 (s, 1H), 8.83 (s, 1H), 7.80-7.66 (m, 4H), 7.06-6.93 (m, 5H), 6.66 (t, J=7.8 Hz, 3H), 5.77 (s, 1H), 5.42 (t, J=7.2 Hz, 1H), 4.31 (s, 1H), 3.35-3.21 (m, 8H), 3.02-2.92 (m, 2H), 2.72-2.57 (m, 5H), 2.50 (s, 3H), 2.23-2.06 (m, 3H), 1.96-1.76 (m, 6H), 1.62-1.51 (m, 6H), 1.37 (s, 9H); [M+H]⁺=863.6.

### Example 110 and 111: 3-(tert-butyl)-N—((R)-1-(4-(7-(9-((1S,3r)-3-(4-((S)-2,6-dioxopiperidin-3-yl)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide and 3-(tert-butyl)-N—((R)-1-(4-(7-(9-((1R,3s)-3-(4-((S)-2,6-dioxopiperidin-3-yl)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide

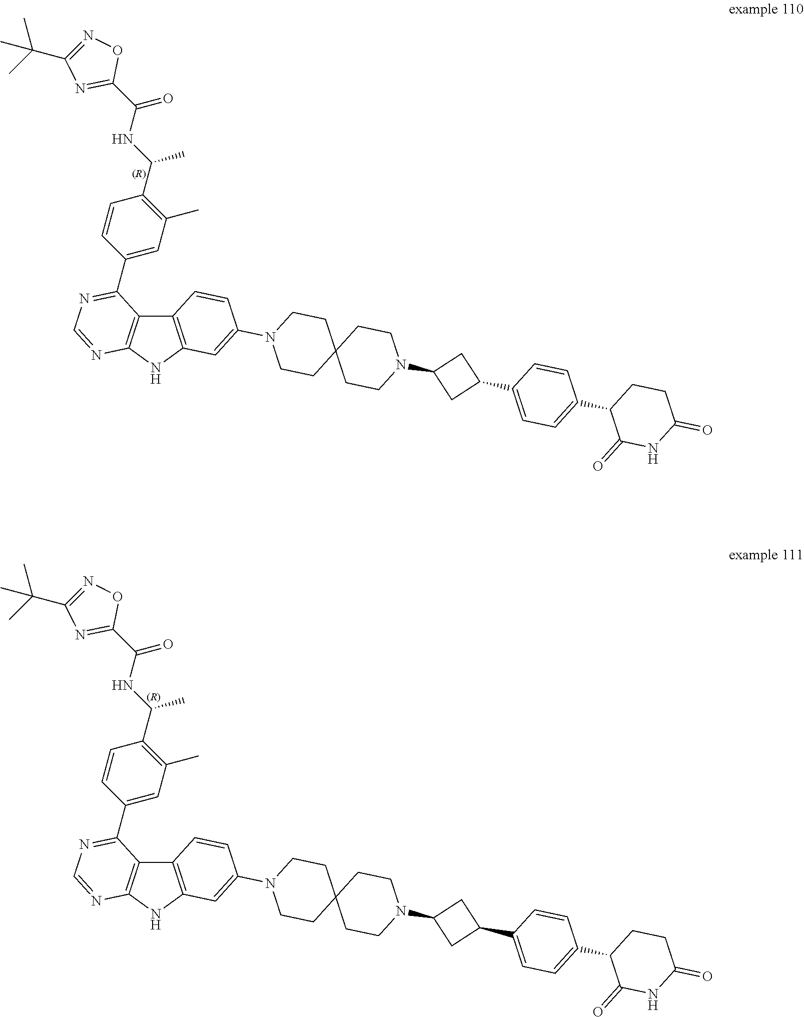

A mixture of (R)—N-(1-(4-(7-(3,9-diazaspiro[5.5]undecan-3-yl)-9H-pyrimido[4,5-b]indol-4-yl)-2-methylphenyl)ethyl)-3-(tert-butyl)-1,2,4-oxadiazole-5-carboxamide (0.08 g, 0.132 mmol) and (S)-3-(4-(3-oxocyclobutyl)phenyl)piperidine-2,6-dione (0.051 g, 0.198 mmol) in 1,2-dichloroethane (4 mL) and HOAc (8 mg) was stirred in a round bottom flask at room temperature for 0.5 hour. To the mixture was added NaBH(OAc)₃ (0.070 g, 0.33 mmol) and stirred at room temperature for 12 hours. Then the mixture was evaporated in vacuum to afford the crude product, which was purified by pre-HPLC to afford example 110 (4.51 mg, 4%) and example 111 (38.76 mg, 35%). Example 110: ¹H NMR (500 MHz, DMSO) δ 12.12 (s, 1H), 10.82 (s, 1H), 9.93 (d, J=7.9 Hz, 1H), 8.80 (s, 1H), 7.81-7.63 (m, 5H), 7.27 (d, J=8.1 Hz, 2H), 7.17 (d, J=8.1 Hz, 2H), 6.91 (d, J=7.6 Hz, 2H), 5.46-5.38 (m, 1H), 3.81 (dd, J=11.4, 5.0 Hz, 1H), 3.40-3.25 (m, 6H), 2.69-2.61 (m, 1H), 2.45-2.15 (m, 10H), 2.08-2.01 (m, 1H), 1.65-1.42 (m, 10H), 1.37 (s, 9H); [M+H]⁺=848.6. Example 111: ¹H NMR (500 MHz, DMSO) δ 12.12 (s, 1H), 10.81 (s, 1H), 9.93 (d, J=7.9 Hz, 1H), 8.80 (s, 1H), 7.81-7.63 (m, 5H), 7.17 (dd, J=25.0, 8.2 Hz, 5H), 6.91 (d, J=8.2 Hz, 2H), 5.46-5.38 (m, 1H), 3.81 (dd, J=11.4, 5.0 Hz, 1H), 3.30-3.15 (m, 5H), 3.16-3.07 (m, 1H), 2.28-2.60 (m, 2H), 2.45-2.25 (m, 7H), 2.21-1.80 (m, 4H), 1.65-1.42 (m, 13H), 1.37 (s, 9H); [M+H]⁺=848.6.

### Example 112 and 113: 3-(tert-butyl)-N—((R)-1-(4-(7-(9-((1R,3r)-3-(4-((R)-2,6-dioxopiperidin-3-yl)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide and 3-(tert-butyl)-N—((R)-1-(4-(7-(9-((1S,3s)-3-(4-((R)-2,6-dioxopiperidin-3-yl)phenyl)cyclobutyl)-3,9-diazaspiro[5.5]undecan-3-yl)-9H-pyrimido[4,5-b]indol-4-yl)-2-methylphenyl)ethyl)-1,2,4-oxadiazole-5-carboxamide

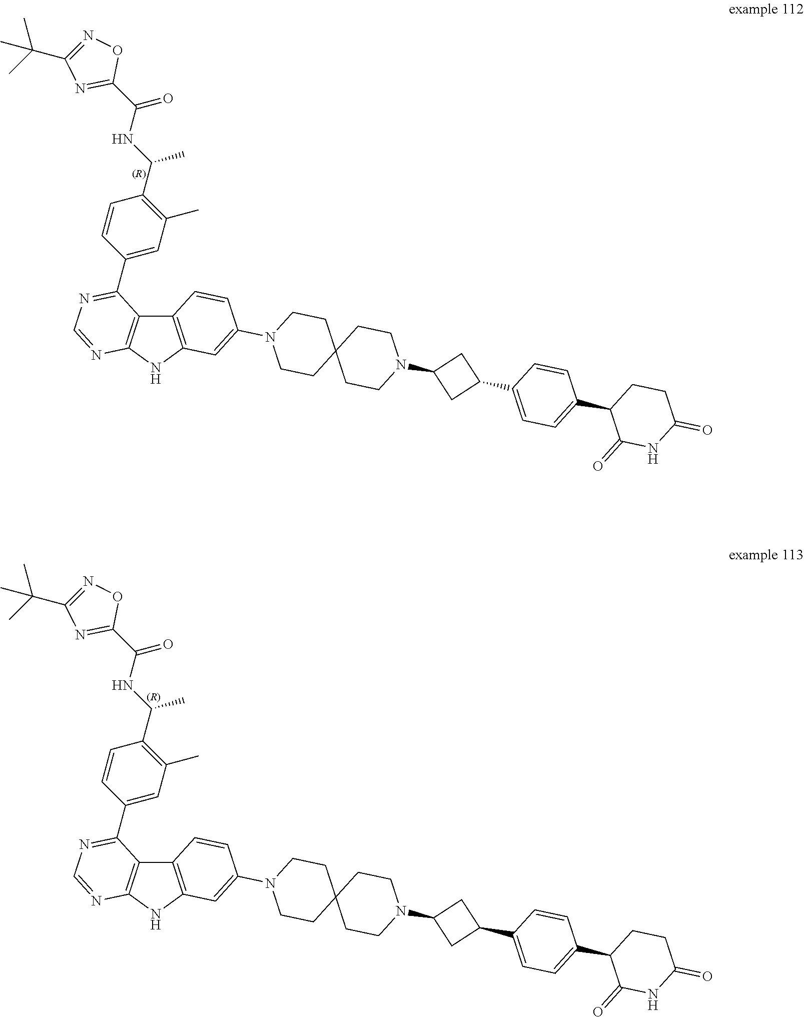

A mixture of (R)—N-(1-(4-(7-(3,9-diazaspiro[5.5]undecan-3-yl)-9H-pyrimido[4,5-b]indol-4-yl)-2-methylphenyl)ethyl)-3-(tert-butyl)-1,2,4-oxadiazole-5-carboxamide (0.08 g, 0.132 mmol) and (R)-3-(4-(3-oxocyclobutyl)phenyl)piperidine-2,6-dione (0.051 g, 0.198 mmol) in 1,2-dichloroethane (4 mL) and HOAc (8 mg) was stirred in a round bottom flask at room temperature for 0.5 hour. To the mixture was added NaBH(OAc)₃ (0.070 g, 0.33 mmol) and stirred in a round bottom flask at room temperature for 12 hours. Then the mixture was evaporated in vacuum to afford the crude product, which was purified by pre-HPLC to afford example 112 (9.28 mg, 8%) and example 113 (61.03 mg, 54%). Example 112: ¹H NMR (500 MHz, DMSO) δ 12.12 (s, 1H), 10.82 (s, 1H), 9.93 (d, J=7.8 Hz, 1H), 8.80 (s, 1H), 7.81-7.64 (m, 4H), 7.26 (d, J=8.1 Hz, 2H), 7.16 (d, J=8.1 Hz, 2H), 6.91 (d, J=8.5 Hz, 2H), 5.46-5.38 (m, 1H), 3.82 (dd, J=11.5, 5.0 Hz, 1H), 3.42-3.25 (m, 6H), 2.96-2.90 (m, 1H), 2.69-2.62 (m, 1H), 2.50 (s, 3H), 2.38-2.27 (m, 6H), 2.20-2.16 (m, 3H), 2.06-2.00 (m, 1H), 1.61-1.45 (m, 11H), 1.37 (s, 9H); [M+H]⁺=848.6. Example 113: ¹H NMR (500 MHz, DMSO) δ 12.12 (s, 1H), 10.81 (s, 1H), 9.93 (d, J=7.9 Hz, 1H), 8.81 (s, 1H), 7.80-7.65 (m, 4H), 7.17 (dd, J=25.5, 8.2 Hz, 4H), 6.91 (d, J=8.1 Hz, 2H), 5.46-5.38 (m, 1H), 3.82 (dd, J=11.4, 4.9 Hz, 1H), 3.29-3.26 (m, 4H), 3.15-3.09 (m, 1H), 2.85 (s, 1H), 2.70-2.61 (m, 1H), 2.50 (s, 3H), 2.45-2.35 (m, 7H), 2.20-2.11 (m, 1H), 2.05-1.99 (m, 1H), 1.92-1.82 (m, 2H), 1.61-1.45 (m, 11H), 1.37 (s, 9H); [M+H]⁺=848.6.

### Example 114: (R)-3-(tert-butyl)-N-(1-(4-(7-(9-((1-(4-(2,4-dioxotetrahydropyrimidin-1(2H)-yl)phenyl)piperidin-4-yl)methyl)-3,9-diazaspiro[5.5]undecan-3-yl)-9H-pyrimido[4,5-b]indol-4-yl)-2-methylphenyl)ethyl)-1,2,4-oxadiazole-5-carboxamide

### Step 1: 3-benzyl 9-(tert-butyl) 3,9-diazaspiro[5.5]undecane-3,9-dicarboxylate

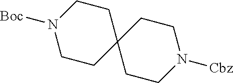 [SMILES: CC(C)(C)OC(=O)N1CCC2(CCN(C(=O)OCc3ccccc3)CC2)CC1]

To a stirred solution of tert-butyl 3,9-diazaspiro[5.5]undecane-3-carboxylate (5.0 g, 19.66 mmol) in DCM (50 mL) were added DIEA (10 mL, 58.97 mmol) and CbzCl (5 mL, 29.48 mmol) dropwise at 0° C. under nitrogen atmosphere. The resulting mixture was stirred for 16 h at room temperature under nitrogen atmosphere and concentrated under reduced pressure. The residue was purified by silica gel column chromatography, eluted with 10%-30% EtOAc in petroleum ether to afford the product (7.6 g, 99%). [M+H]⁺=389.2.

### Step 2: benzyl 3,9-diazaspiro[5.5]undecane-3-carboxylate

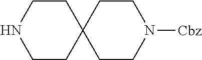 [SMILES: O=C(OCc1ccccc1)N1CCC2(CCNCC2)CC1]

To a stirred solution of 3-benzyl 9-(tert-butyl) 3,9-diazaspiro[5.5]undecane-3,9-dicarboxylate (7.6 g, 19.56 mmol) in DCM (70 mL) was added 4 M HCl in 1,4-dioxane (70 mL) dropwise at room temperature. The resulting mixture was concentrated under reduced pressure. The residue was basified to pH 8 with saturated Na₂CO₃ (aq.). The resulting mixture was extracted with EtOAc (5×150 mL). The combined organic layers were washed with brine (100 mL) and dried over anhydrous Na₂SO₄. After filtration, the filtrate was concentrated under reduced pressure. This resulted in the product (4.2 g, 74%). [M+H]⁺=289.2.

### Step 3: benzyl 9-(4-((2-(trimethylsilyl)ethoxy)methoxy)-9-((2-(trimethylsilyl)ethoxy)methyl)-9H-pyrimido[4,5-b]indol-7-yl)-3,9-diazaspiro[5.5]undecane-3-carboxylate

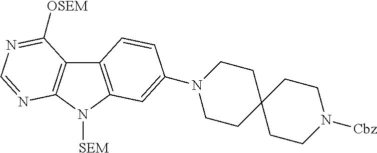

To a stirred mixture of 7-bromo-4-((2-(trimethylsilyl)ethoxy)methoxy)-9-((2-(trimethylsilyl)ethoxy)methyl)-9H-pyrimido[4,5-b]indole (6.5 g, 12.39 mmol) and benzyl 3,9-diazaspiro[5.5]undecane-3-carboxylate (4.2 g, 14.87 mmol) in dioxane (130 mL) were added Cs₂CO₃ (8.0 g, 24.78 mmol), XPhos (1.2 g, 2.48 mmol) and XPhos Pd G₃ (1.0 g, 1.24 mmol) at room temperature under nitrogen atmosphere. The resulting mixture was stirred for 16 h at 100° C. under nitrogen atmosphere. The mixture was allowed to cool down to room temperature and concentrated under reduced pressure. The residue was purified by silica gel column chromatography, eluted with 30%-50% EtOAc in petroleum ether to afford the product (3.2 g, 35%). [M+H]⁺=732.5.

### Step 4: benzyl 9-(4-oxo-4,9-dihydro-3H-pyrimido[4,5-b]indol-7-yl)-3,9-diazaspiro[5.5]undecane-3-carboxylate

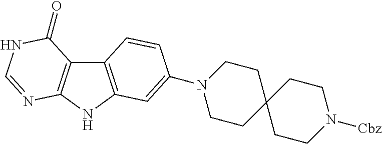 [SMILES: O=C(OCc1ccccc1)N1CCC2(CC1)CCN(c1ccc3c(c1)[nH]c1nc[nH]c(=O)c13)CC2]

A solution of benzyl 9-(4-((2-(trimethylsilyl)ethoxy)methoxy)-9-((2-(trimethylsilyl)ethoxy)methyl)-9H-pyrimido[4,5-b]indol-7-yl)-3,9-diazaspiro[5.5]undecane-3-carboxylate (3.2 g, 4.37 mmol) in TFA (60 mL) was stirred for 2 h at room temperature. The resulting mixture was concentrated under reduced pressure. To the residue were added DCM (70 mL), MeOH (10 mL) and basified to pH 8 with NH₃·H₂O. The resulting mixture was stirred for 24 h at room temperature and concentrated under reduced pressure. To the resulting mixture was added water (80 mL) and filtered. The filter cake was washed with water (3×10 mL) and Et₂O (3×10 mL). The solid was dried under infrared light. This resulted in the product (1.5 g, 72%). [M+H]⁺=472.4.

### Step 5: benzyl 9-(4-chloro-9H-pyrimido[4,5-b]indol-7-yl)-3,9-diazaspiro[5.5]undecane-3-carboxylate

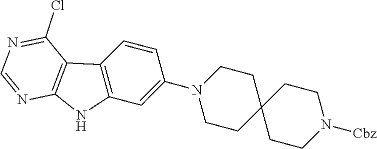 [SMILES: O=C(OCc1ccccc1)N1CCC2(CC1)CCN(c1ccc3c(c1)[nH]c1ncnc(Cl)c13)CC2]

A mixture of benzyl 9-(4-oxo-4,9-dihydro-3H-pyrimido[4,5-b]indol-7-yl)-3,9-diazaspiro[5.5]undecane-3-carboxylate (1.4 g, 2.97 mmol) and POCl₃ (2.8 mL, 30.05 mmol) in dioxane (30 mL) was stirred for 1 h at 100° C. The mixture was allowed to cool down to room temperature and concentrated under reduced pressure. The reaction was quenched with water/ice at 0° C. The mixture was basified to pH 8 with saturated NaHCO₃ (aq.). The resulting mixture was extracted with EtOAc (3×200 mL). The combined organic layers were washed with brine (200 mL) and dried over anhydrous Na₂SO₄. After filtration, the filtrate was concentrated under reduced pressure. This resulted in the product (740 mg, 51%). [M+H]⁺=490.2.

### Step 6: benzyl (R)-9-(4-(4-(1-(3-(tert-butyl)-1,2,4-oxadiazole-5-carboxamido)ethyl)-3-methylphenyl)-9H-pyrimido[4,5-b]indol-7-yl)-3,9-diazaspiro[5.5]undecane-3-carboxylate

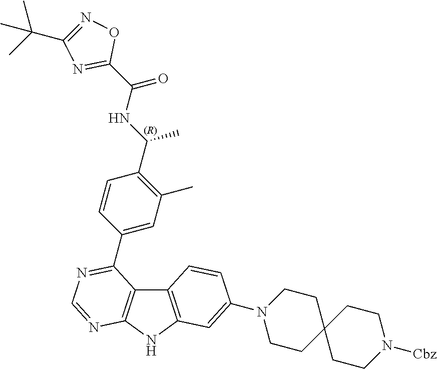

To a stirred mixture of benzyl 9-(4-chloro-9H-pyrimido[4,5-b]indol-7-yl)-3,9-diazaspiro[5.5]undecane-3-carboxylate (740 mg, 1.510 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (749 mg, 1.812 mmol) in dioxane (15 mL) and H₂O (3 mL) were added K₃PO₄ (641 mg, 3.020 mmol) and Pd(dppf)Cl₂ (110 mg, 0.151 mmol) at room temperature under nitrogen atmosphere. The resulting mixture was stirred for 16 h at 80° C. under nitrogen atmosphere. The mixture was allowed to cool down to room temperature and concentrated under reduced pressure. The residue was purified by silica gel column chromatography, eluted with 5%-10% MeOH in DCM to afford the product (500 mg, 44%). [M+H]⁺=741.3.

### Step 7: (R)—N-(1-(4-(7-(3,9-diazaspiro[5.5]undecan-3-yl)-9H-pyrimido[4,5-b]indol-4-yl)-2-methylphenyl)ethyl)-3-(tert-butyl)-1,2,4-oxadiazole-5-carboxamide

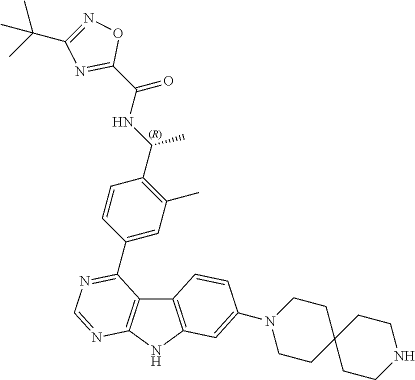

A solution of benzyl (R)-9-(4-(4-(1-(3-(tert-butyl)-1,2,4-oxadiazole-5-carboxamido)ethyl)-3-methylphenyl)-9H-pyrimido[4,5-b]indol-7-yl)-3,9-diazaspiro[5.5]undecane-3-carboxylate (500 mg, 0.675 mmol) in TFA (10 mL) was stirred for 16 h at 75° C. The mixture was allowed to cool down to room temperature and concentrated under reduced pressure. The residue was purified by reverse phase flash chromatography with the following conditions: column, C18; mobile phase A: FA in water (0.1%), mobile phase B: ACN, 10% to 40% gradient in 20 min; Detector, UV 254/220 nm. This resulted in the product (335 mg, 81%). ¹H NMR (400 MHz, DMSO) δ 12.16 (s, 1H), 9.94 (d, J=7.9 Hz, 1H), 8.81 (s, 1H), 8.35 (s, 1H), 7.91-7.43 (m, 4H), 6.94-6.90 (m, 2H), 5.47-5.38 (m, 1H), 3.30-3.23 (m, 5H), 2.54-2.51 (m, 3H), 2.97 (s, 4H), 1.64-1.56 (m, 10H), 1.38 (s, 9H); [M+H]⁺=607.5.

### Step 8: (R)-3-(tert-butyl)-N-(1-(4-(7-(9-((1-(4-(2,4-dioxotetrahydropyrimidin-1(2H)-yl)phenyl)piperidin-4-yl)methyl)-3,9-diazaspiro[5.5]undecan-3-yl)-9H-pyrimido[4,5-b]indol-4-yl)-2-methylphenyl)ethyl)-1,2,4-oxadiazole-5-carboxamide

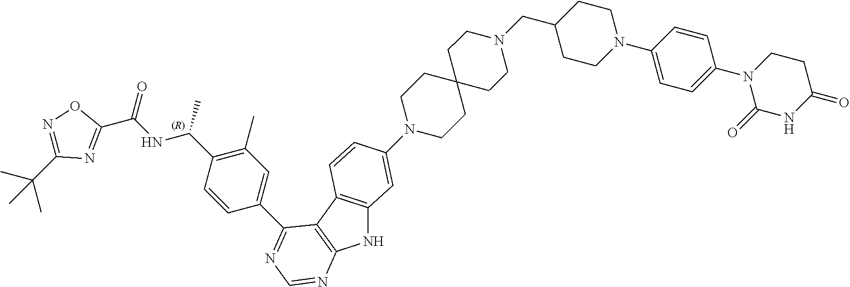

A mixture of (R)—N-(1-(4-(7-(3,9-diazaspiro[5.5]undecan-3-yl)-9H-pyrimido[4,5-b]indol-4-yl)-2-methylphenyl)ethyl)-3-(tert-butyl)-1,2,4-oxadiazole-5-carboxamide (0.08 g, 0.132 mmol) and 1-(4-(2,4-dioxotetrahydropyrimidin-1(2H)-yl)phenyl)piperidine-4-carbaldehyde (0.060 g, 0.199 mmol) in 1,2-dichloroethane (4 mL) and HOAc (8 mg) was stirred in a round bottom flask at room temperature for 0.5 hour. To the mixture was added NaBH(OAc)₃ (0.070 g, 0.33 mmol) and stirred at room temperature for 12 hours. Then the mixture was evaporated in vacuum to afford the crude product, which was purified by pre-HPLC to afford the product (0.036 g, 31%). ¹H NMR (400 MHz, DMSO) δ 12.12 (s, 1H), 10.25 (s, 1H), 9.93 (d, J=7.9 Hz, 1H), 8.80 (s, 1H), 7.80-7.63 (m, 4H), 7.12 (d, J=8.6 Hz, 2H), 6.91 (dd, J=8.1, 4.7 Hz, 4H), 5.41 (d, J=7.3 Hz, 1H), 3.71-3.61 (m, 4H), 3.25 (s, 4H), 2.72-2.60 (m, 4H), 2.36 (s, 4H), 2.19 (s, 2H), 1.77 (d, J=11.8 Hz, 2H), 1.58-1.45 (m, 10H), 1.37 (s, 9H), 1.25-1.14 (m, 4H); [M+H]⁺=892.7.

### Example 115 and 116: N-((1R)-1-(4-(7-(4-((1s,3s)-3-(4-((2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N-((1R)-1-(4-(7-(4-((1r,3r)-3-(4-((2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: 2-(4-bromophenyl)-5,8-dioxaspiro[3.4]octane

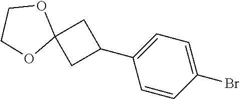 [SMILES: Brc1ccc(C2CC3(C2)OCCO3)cc1]

A solution of 3-(4-bromophenyl)cyclobutan-1-one (10.00 g, 44.428 mmol), 2-dihydroxyethane (10 mL, 177.706 mmol) and 4-methylbenzene-1-sulfonic acid hydrate (845 mg, 4.443 mmol) in toluene (200 mL) was stirred for 16 h at 110° C. through a Dean Stark to separate the water under nitrogen atmosphere. The mixture was allowed to cool down to room temperature and concentrated under reduced pressure. To the resulting mixture was added water (200 mL) and extracted with EtOAc (3×200 mL). The combined organic layers were washed with brine (200 mL) and dried over anhydrous Na₂SO₄. After filtration, the filtrate was concentrated under reduced pressure. The residue was purified by silica gel column chromatography, eluted with PE/EtOAc (20:1 to 4:1) to afford the product (11.5 g, 96%). [M+H]⁺=269.0.

### Step 2: tert-butyl (4-(5,8-dioxaspiro[3.4]octan-2-yl)phenyl)carbamate

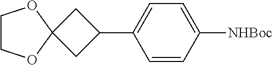 [SMILES: CC(C)(C)OC(=O)Nc1ccc(C2CC3(C2)OCCO3)cc1]

To a stirred mixture of 2-(4-bromophenyl)-5,8-dioxaspiro[3.4]octane (6 g, 22.293 mmol) and tert-butyl carbamate (3.9 g, 33.462 mmol) in dioxane (120 mL) were added Cs₂CO₃ (14.5 g, 44.595 mmol), XPhos (2.1 g, 4.468 mmol) and XPhos Pd G3 (1.9 g, 2.233 mmol) at room temperature under nitrogen atmosphere. The resulting mixture was stirred for 16 h at 100° C. under nitrogen atmosphere. The mixture was allowed to cool down to room temperature and concentrated under reduced pressure. The residue was purified by silica gel column chromatography, eluted with PE/EtOAc (25:1 to 5:1) to afford the product (6.5 g, 95%). [M+Na]⁺=328.2.

### Step 3: 4-(5,8-dioxaspiro[3.4]octan-2-yl)aniline

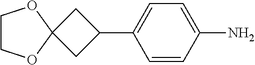 [SMILES: Nc1ccc(C2CC3(C2)OCCO3)cc1]

A solution of tert-butyl (4-(5,8-dioxaspiro[3.4]octan-2-yl)phenyl)carbamate (6.3 g, 20.565 mmol) and TBAF (102.82 mL, 393.250 mmol) in THF (70 mL) was stirred for 16 h at 70° C. The mixture was allowed to cool down to room temperature and concentrated under reduced pressure. The residue was purified by silica gel column chromatography, eluted with PE/EA (1:1) to afford the product (4 g, 95%). [M+H]⁺=206.1.

### Step 4: 3-((4-(5,8-dioxaspiro[3.4]octan-2-yl)phenyl)amino)piperidine-2,6-dione

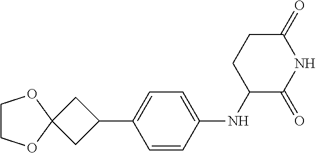 [SMILES: O=C1CCC(Nc2ccc(C3CC4(C3)OCCO4)cc2)C(=O)N1]

A mixture of 4-(5,8-dioxaspiro[3.4]octan-2-yl)aniline (4 g, 19.536 mmol), 3-bromopiperidine-2,6-dione (4.5 g, 23.436 mmol) and NaHCO₃ (3.3 g, 39.045 mmol) in DMF (80 mL) was stirred for 16 h at 80° C. under nitrogen atmosphere. The mixture was allowed to cool down to room temperature and diluted with water (120 mL). The resulting mixture was extracted with EtOAc (3×200 mL). The combined organic layers were washed with brine (3×150 mL) and dried over anhydrous Na₂SO₄. After filtration, the filtrate was concentrated under reduced pressure. The residue was purified by silica gel column chromatography, eluted with PE/EA (5:1 to 1:1) to afford the product (3.4 g, 55%). [M+H]⁺=317.1.

### Step 5: 3-((4-(3-oxocyclobutyl)phenyl)amino)piperidine-2,6-dione

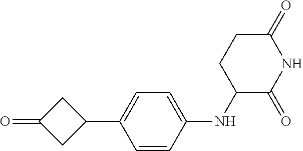 [SMILES: O=C1CC(c2ccc(NC3CCC(=O)NC3=O)cc2)C1]

A solution of 3-((4-(5,8-dioxaspiro[3.4]octan-2-yl)phenyl)amino)piperidine-2,6-dione (4.2 g, 13.276 mmol) and HCl (aq.) (40 mL, 1167.832 mmol, 2 M) in THF (40 mL) was stirred for 2 h at 55° C. The mixture was allowed to cool down to 0° C. The residue was basified to pH 6 with NaHCO₃ and diluted with water (100 mL). The resulting mixture was extracted with EtOAc (3×150 mL). The combined organic layers were washed with brine (2×100 mL) and dried over anhydrous Na₂SO₄. After filtration, the filtrate was concentrated under reduced pressure. The residue was purified by silica gel column chromatography, eluted with PE/EA (10:1 to 1:1) to afford the product (2.2 g, 62%). ¹H NMR (400 MHz, DMSO) δ 10.77 (s, 1H), 7.13-7.05 (m, 2H), 6.70-6.62 (m, 2H), 5.75 (d, J=7.6 Hz, 1H), 4.34-4.25 (m, 1H), 3.55-3.45 (m, 1H), 3.44-3.31 (m, 2H), 3.17-3.04 (m, 2H), 2.80-2.68 (m, 1H), 2.59 (dt, J=17.6, 4.3 Hz, 1H), 2.17-2.05 (m, 1H), 1.87 (qd, J=12.0, 4.7 Hz, 1H); [M+H]⁺=273.1.

### Step 6: N-((1R)-1-(4-(7-(4-((1s,3s)-3-(4-((2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N-((1R)-1-(4-(7-(4-((1r,3r)-3-(4-((2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

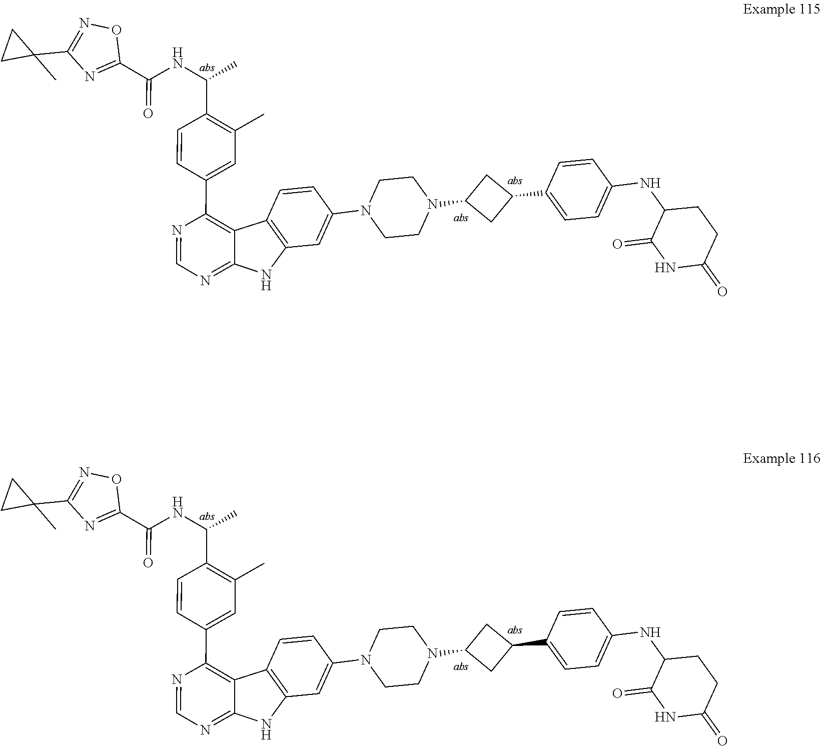 [SMILES: Cc1cc(-c2ncnc3[nH]c4cc(N5CCN([C@H]6C[C@@H](c7ccc(NC8CCC(=O)NC8=O)cc7)C6)CC5)ccc4c23)ccc1[C@@H](C)NC(=O)c1nc(C2(C)CC2)no1 ; Cc1cc(-c2ncnc3[nH]c4cc(N5CCN([C@H]6C[C@H](c7ccc(NC8CCC(=O)NC8=O)cc7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (100 mg, crude), 3-((4-(3-oxocyclobutyl)phenyl)amino)piperidine-2,6-dione (60 mg, 0.373 mmol) in tetrahydrofuran (3 mL) and N,N-dimethylformamide (3 mL) was stirred at room temperature for 5 mins. Then Ti(OiPr)₄ (0.2 mL) was added. The mixture was stirred at room temperature overnight. Then NaBH(OAc)₃ (197 mg, 0.93 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give crude product, which was further purified by prep-HPLC to afford example 115 (3.45 mg, 2.3%) and example 116 (0.56 mg, 0.3%). Example 115: ¹H NMR (500 MHz, DMSO) δ 12.15 (s, 1H), 10.76 (s, 1H), 9.85 (d, J=4.0 Hz, 1H), 8.81 (d, J=2.0 Hz, 1H), 7.80-7.65 (m, 4H), 7.05-6.83 (m, 4H), 6.62 (d, J=8.0 Hz, 2H), 5.67 (d, J=8.0 Hz, 1H), 5.39 (s, 1H), 4.28 (s, 1H), 3.26 (s, 4H), 2.98 (s, 1H), 2.78-2.53 (m, 6H), 2.46-2.44 (m, 4H), 2.43-2.35 (m, 2H), 2.08 (s, 1H), 1.90-1.76 (m, 3H), 1.56 (d, J=4.0 Hz, 3H), 1.48 (s, 3H), 1.20 (s, 2H), 0.98 (s, 2H); [M+H]⁺=793.7. Example 116: ¹H NMR (500 MHz, DMSO) δ 12.16 (s, 1H), 10.77 (s, 1H), 9.86 (d, J=8.0 Hz, 1H), 8.82 (s, 1H), 8.29 (s, 1H), 7.86-7.60 (m, 4H), 7.03 (d, J=8.0 Hz, 2H), 6.94 (s, 2H), 6.64 (d, J=8.0 Hz, 2H), 5.68 (d, J=8.0 Hz, 1H), 5.39 (t, J=8.0 Hz, 1H), 4.28 (s, 1H), 2.91-2.86 (m, 2H), 2.80-2.70 (m, 2H), 2.66-2.53 (m, 4H), 2.38-2.29 (m, 6H), 2.15-2.05 (m, 4H), 1.92-1.80 (m, 2H), 1.56 (d, J=8.0 Hz, 2H), 1.49 (s, 3H), 1.26-1.17 (m, 3H), 0.99 (d, J=2.0 Hz, 2H); [M+H]⁺=793.7.

### Example 117 and 118: N—((R)-1-(4-(7-(4-((1R,3r)-3-(4-(((R)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N—((R)-1-(4-(7-(4-((1S,3s)-3-(4-(((R)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: (R)-3-((4-(3-oxocyclobutyl)phenyl)amino)piperidine-2,6-dione

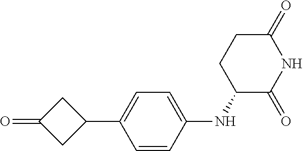 [SMILES: O=C1CC(c2ccc(N[C@@H]3CCC(=O)NC3=O)cc2)C1]

(R)-3-((4-(3-Oxocyclobutyl)phenyl)amino)piperidine-2,6-dione (9.5 g) was separated by Prep-SFC with the following conditions (Column: CHIRALPAK IH, 3*25 cm, 5 μm; Mobile Phase A: CO₂, Mobile Phase B: MeOH (0.1% 2 M NH₃-MeOH); Flow rate: 70 mL/min; Gradient: isocratic 50% B; Column Temperature (° C.): 35; Back Pressure (bar): 100; Wave Length: 220 nm; RT1 (min): 4.81; RT2 (min): 8.35. The fractions of second peak (RT2: 8.35 min) were collected and evaporated in vacuo and then lyophilized overnight to give the product (3.7 g, 39%). ¹H NMR (400 MHz, CDCl₃) δ 8.21 (s, 1H), 7.20-7.11 (m, 2H), 6.87-6.60 (m, 2H), 4.79-4.75 (m, 1H), 4.11 (dd, J=12.5, 4.8 Hz, 1H), 3.71-3.56 (m, 1H), 3.56-3.39 (m, 2H), 3.31-3.13 (m, 2H), 2.96-2.75 (m, 2H), 2.63-2.54 (m, 1H), 2.01-1.88 (m, 1H); [M+H]⁺=273.1.

### Step 2: N—((R)-1-(4-(7-(4-((1R,3r)-3-(4-(((R)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N—((R)-1-(4-(7-(4-((1S,3s)-3-(4-(((R)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

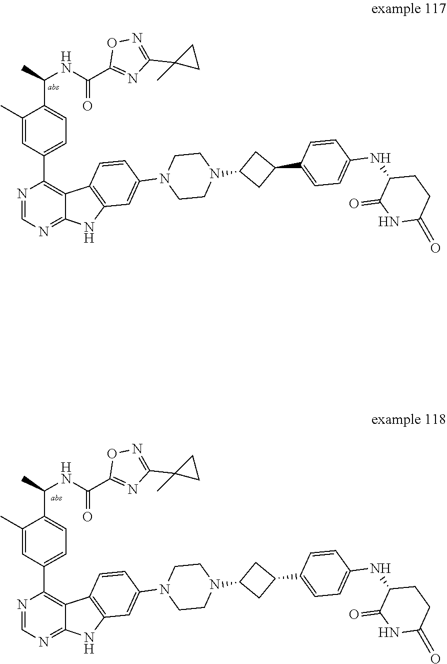 [SMILES: Cc1cc(-c2ncnc3[nH]c4cc(N5CCN([C@H]6C[C@H](c7ccc(N[C@@H]8CCC(=O)NC8=O)cc7)C6)CC5)ccc4c23)ccc1[C@@H](C)NC(=O)c1nc(C2(C)CC2)no1 ; Cc1cc(-c2ncnc3[nH]c4cc(N5CCN([C@H]6C[C@@H](c7ccc(N[C@@H]8CCC(=O)NC8=O)cc7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (0.24 g, 0.447 mmol) and (R)-3-((4-(3-oxocyclobutyl)phenyl)amino)piperidine-2,6-dione (0.183 g, 0.671 mmol) in 1,2-dichloroethane (10 mL) and HOAc (27 mg) was stirred in a round bottom flask at room temperature for 0.5 hour. To the mixture was added NaBH(OAc)₃ (0.143 g, 0.671 mmol) and stirred at room temperature for 12 hours. Then the mixture was evaporated in vacuum to afford the crude product, which was purified by pre-HPLC to afford example 117 (21.62 mg, 6%) and example 118 (100.03 mg, 28%). Example 117: ¹H NMR (500 MHz, DMSO) δ 12.16 (s, 1H), 10.77 (s, 1H), 9.86 (d, J=8.0 Hz, 1H), 8.82 (s, 1H), 7.81-7.72 (m, 2H), 7.68 (dd, J=8.7, 3.7 Hz, 2H), 7.03 (d, J=8.5 Hz, 2H), 6.93 (d, J=5.5 Hz, 2H), 6.64 (d, J=8.5 Hz, 2H), 5.68 (d, J=7.5 Hz, 1H), 5.45-5.34 (m, 1H), 4.33-4.24 (m, 1H), 3.27 (s, 5H), 2.95-2.84 (m, 1H), 2.78-2.71 (m, 1H), 2.65-2.53 (m, 2H), 2.50 (s, 3H), 2.47 (s, 3H), 2.38-2.26 (m, 2H), 2.15-2.07 (m, 3H), 1.92-1.82 (m, 1H), 1.56 (d, J=6.9 Hz, 3H), 1.49 (s, 3H), 1.21 (dd, J=9.3, 6.0 Hz, 2H), 0.99 (d, J=2.7 Hz, 2H); [M+H]⁺=793.4. Example 118: ¹H NMR (500 MHz, DMSO) δ 12.16 (s, 1H), 10.76 (s, 1H), 9.86 (d, J=7.9 Hz, 1H), 7.81-7.65 (m, 4H), 6.95 (dd, J=22.4, 7.3 Hz, 4H), 6.62 (d, J=8.5 Hz, 2H), 5.67 (d, J=7.6 Hz, 1H), 5.42-5.38 (m, 1H), 4.32-4.24 (m, 1H), 3.30-3.22 (m, 5H), 3.03-2.94 (m, 1H), 2.80-2.64 (m, 2H), 2.62-2.54 (m, 2H), 2.50 (s, 3H), 2.48-2.36 (m, 6H), 2.14-2.07 (m, 1H), 1.91-1.75 (m, 3H), 1.56 (d, J=6.9 Hz, 3H), 1.49 (s, 3H), 1.21 (dd, J=9.3, 6.0 Hz, 2H), 0.99 (d, J=2.7 Hz, 2H); [M+H]⁺=793.4.

### Example 119 and 120: N—((R)-1-(4-(7-(4-((1S,3r)-3-(4-(((S)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N—((R)-1-(4-(7-(4-((1R,3s)-3-(4-(((S)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: (S)-3-((4-(3-oxocyclobutyl)phenyl)amino)piperidine-2,6-dione

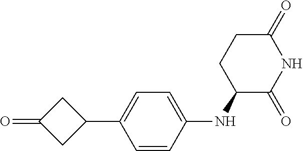 [SMILES: O=C1CC(c2ccc(N[C@H]3CCC(=O)NC3=O)cc2)C1]

(S)-3-((4-(3-oxocyclobutyl)phenyl)amino)piperidine-2,6-dione (9.5 g) was separated by Prep-SFC with the following conditions (Column: CHIRALPAK IH, 3*25 cm, 5 m; Mobile Phase A: CO₂, Mobile Phase B: MeOH (0.1% 2 M NH₃-MeOH); Flow rate: 70 mL/min; Gradient: isocratic 50% B; Column Temperature (° C.): 35; Back Pressure (bar): 100; Wave Length: 220 nm; RT1 (min): 4.81; RT2 (min): 8.35. The fractions of first peak (RT1: 4.81 min) were collected and evaporated in vacuo and then lyophilized overnight to give the product (3.9 g, 41%). ¹H NMR (400 MHz, CDCl₃) δ 8.21 (s, 1H), 7.20-7.11 (m, 2H), 6.87-6.60 (m, 2H), 4.79-4.75 (m, 1H), 4.11 (dd, J=12.5, 4.8 Hz, 1H), 3.71-3.56 (m, 1H), 3.56-3.39 (m, 2H), 3.31-3.13 (m, 2H), 2.96-2.75 (m, 2H), 2.63-2.54 (m, 1H), 2.01-1.88 (m, 1H); [M+H]⁺=273.1.

### Step 2: N—((R)-1-(4-(7-(4-((1S,3r)-3-(4-(((S)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide and N—((R)-1-(4-(7-(4-((1R,3s)-3-(4-(((S)-2,6-dioxopiperidin-3-yl)amino)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

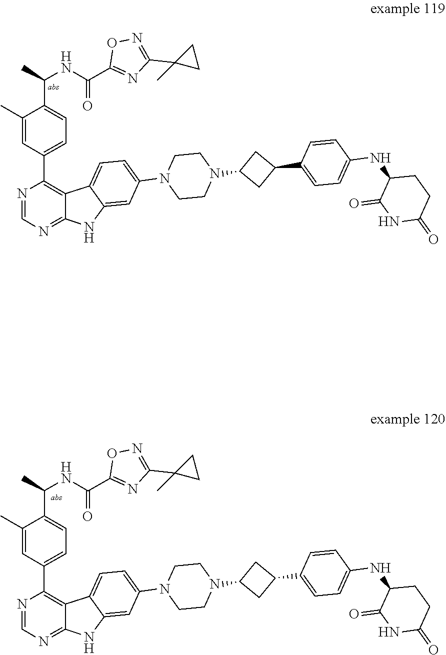 [SMILES: Cc1cc(-c2ncnc3[nH]c4cc(N5CCN([C@H]6C[C@H](c7ccc(N[C@H]8CCC(=O)NC8=O)cc7)C6)CC5)ccc4c23)ccc1[C@@H](C)NC(=O)c1nc(C2(C)CC2)no1 ; Cc1cc(-c2ncnc3[nH]c4cc(N5CCN([C@H]6C[C@@H](c7ccc(N[C@H]8CCC(=O)NC8=O)cc7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (0.24 g, 0.447 mmol) and (S)-3-((4-(3-oxocyclobutyl)phenyl)amino)piperidine-2,6-dione (0.183 g, 0.671 mmol) in 1,2-dichloroethane (10 mL) and HOAc (27 mg) was stirred in a round bottom flask at room temperature for 0.5 hour. To the mixture was added NaBH(OAc)₃ (0.143 g, 0.671 mmol) and stirred at room temperature for 12 hours. Then the mixture was evaporated in vacuum to afford the crude product, which was purified by pre-HPLC to afford example 119 (18.50 mg, 5%) and example 120 (94.75 mg, 27%). Example 119: ¹H NMR (500 MHz, DMSO) δ 12.17 (s, 1H), 10.77 (s, 1H), 9.86 (d, J=8.0 Hz, 1H), 8.82 (s, 1H), 7.81-7.64 (m, 4H), 7.03 (d, J=8.5 Hz, 2H), 6.93 (d, J=5.5 Hz, 2H), 6.64 (d, J=8.5 Hz, 2H), 5.68 (d, J=7.5 Hz, 1H), 5.43-5.37 (m, 1H), 4.34-4.25 (m, 1H), 3.30-3.21 (m, 6H), 2.94-2.84 (m, 1H), 2.78-2.71 (m, 1H), 2.65-2.54 (m, 2H), 2.50 (s, 3H), 2.48-2.41 (m, 4H), 2.38-2.28 (m, 2H), 2.15-2.05 (m, 3H), 1.92-1.82 (m, 1H), 1.56 (d, J=7.0 Hz, 3H), 1.49 (s, 3H), 1.21 (dd, J=9.5, 6.3 Hz, 2H), 0.99 (d, J=2.7 Hz, 2H); [M+H]⁺=793.4. Example 120: ¹H NMR (500 MHz, DMSO) δ 12.16 (s, 1H), 10.77 (s, 1H), 9.86 (d, J=7.9 Hz, 1H), 8.82 (s, 1H), 7.80-7.65 (m, 4H), 6.95 (dd, J=22.4, 7.5 Hz, 4H), 6.62 (d, J=8.5 Hz, 2H), 5.68 (d, J=7.6 Hz, 1H), 5.41 (dd, J=15.9, 8.7 Hz, 1H), 4.33-4.24 (m, 1H), 3.30-3.21 (m, 5H), 3.05-2.95 (m, 1H), 2.78-2.52 (m, 3H), 2.50 (s, 3H), 2.48-2.35 (m, 6H), 2.10 (dd, J=17.3, 4.4 Hz, 1H), 1.91-1.75 (m, 3H), 1.56 (d, J=6.9 Hz, 3H), 1.49 (s, 3H), 1.27-1.17 (m, 2H), 0.99 (d, J=2.7 Hz, 2H); [M+H]⁺=793.4.

### Example 121: N-((1R)-1-(4-(7-(4-(1′-(2-(2,6-dioxopiperidin-3-yl)-1,3-dioxoisoindolin-5-yl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: 2-(2,6-dioxo-1-((2-(trimethylsilyl)ethoxy)methyl)piperidin-3-yl)-5-fluoroisoindoline-1,3-dione

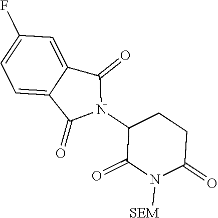 [SMILES: C[Si](C)(C)CCOCN1C(=O)CCC(N2C(=O)c3ccc(F)cc3C2=O)C1=O]

To a stirred solution of 2-(2,6-dioxopiperidin-3-yl)-5-fluoroisoindole-1,3-dione (15.0 g, 54.30 mmol) and DIEA (47.3 mL, 271.52 mmol) in DMF (250 mL) was added SEM-Cl (14.4 mL, 81.46 mmol) dropwise at 0° C. The resulting mixture was stirred for 16 h at room temperature. The resulting mixture was diluted with water (800 mL) and extracted with EtOAc (3×500 mL). The combined organic layers were washed with brine (3×300 mL) and dried over anhydrous Na₂SO₄. After filtration, the filtrate was concentrated under reduced pressure. The residue was purified by silica gel column chromatography, eluted with 20%-40% EtOAc in petroleum ether to afford the product (21 g, 95%).

### Step 2: 2-(2,6-dioxo-1-((2-(trimethylsilyl)ethoxy)methyl)piperidin-3-yl)-5-(3-hydroxyazetidin-1-yl)isoindoline-1,3-dione

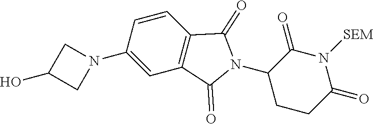 [SMILES: C[Si](C)(C)CCOCN1C(=O)CCC(N2C(=O)c3ccc(N4CC(O)C4)cc3C2=O)C1=O]

A solution of 2-(2,6-dioxo-1-((2-(trimethylsilyl)ethoxy)methyl)piperidin-3-yl)-5-fluoroisoindoline-1,3-dione (20.0 g, 49.202 mmol), azetidin-3-ol hydrochloride (8.1 g, 73.803 mmol) and DIEA (34.3 mL, 196.808 mmol) in DMSO (400 mL) was stirred for 16 h at 110° C. The mixture was allowed to cool down to room temperature. The resulting mixture was diluted with water (800 mL) and extracted with EtOAc (3×500 mL). The combined organic layers were washed with brine (3×400 mL) and dried over anhydrous Na₂SO₄. After filtration, the filtrate was concentrated under reduced pressure. The residue was purified by silica gel column chromatography, eluted with 40%-60% EtOAc in petroleum ether to afford the product (21 g, 92%). [M+Na]⁺=482.1.

### Step 3: 2-(2,6-dioxo-1-((2-(trimethylsilyl)ethoxy)methyl)piperidin-3-yl)-5-(3-oxoazetidin-1-yl)isoindoline-1,3-dione

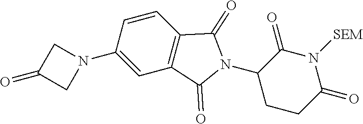 [SMILES: C[Si](C)(C)CCOCN1C(=O)CCC(N2C(=O)c3ccc(N4CC(=O)C4)cc3C2=O)C1=O]

To a stirred mixture of 2-(2,6-dioxo-1-((2-(trimethylsilyl)ethoxy)methyl)piperidin-3-yl)-5-(3-hydroxyazetidin-1-yl)isoindoline-1,3-dione (10 g, 21.759 mmol) in CH₂Cl₂ (200 mL) was added DMP (18.4 g, 43.518 mmol) in portions at room temperature. The resulting mixture was stirred for 16 h at room temperature. To the resulting mixture was added water (300 mL) and extracted with CH₂Cl₂ (3×300 mL). The combined organic layers were washed with brine (2×300 mL) and dried over anhydrous Na₂SO₄. After filtration, the filtrate was concentrated under reduced pressure. The residue was purified by silica gel column chromatography, eluted with 30%-45% EtOAc in petroleum ether to afford the product (7.1 g, 71%). [M+Na]⁺=480.3.

### Step 4: 2-(2,6-dioxo-1-((2-(trimethylsilyl)ethoxy)methyl)piperidin-3-yl)-5-(3-hydroxy-[1,3′-biazetidin]-1′-yl)isoindoline-1,3-dione

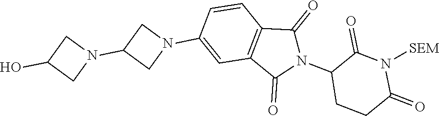 [SMILES: C[Si](C)(C)CCOCN1C(=O)CCC(N2C(=O)c3ccc(N4CC(N5CC(O)C5)C4)cc3C2=O)C1=O]

A mixture of 2-(2,6-dioxo-1-((2-(trimethylsilyl)ethoxy)methyl)piperidin-3-yl)-5-(3-oxoazetidin-1-yl)isoindoline-1,3-dione (6.5 g, 14.21 mmol), azetidin-3-ol hydrochloride (2.3 g, 21.31 mmol) and NaOAc (1.7 g, 21.31 mmol) in DCE (130 mL) was stirred for 1 h at room temperature. To the above mixture was added NaBH(OAc)₃ (4.5 g, 21.31 mmol) at room temperature. The resulting mixture was stirred for additional 16 h at 30° C. and concentrated under reduced pressure. The residue was purified by silica gel column chromatography, eluted with 5%-8% MeOH in CH₂Cl₂ to afford the product (2.6 g, 35%). [M+H]⁺=515.4.

### Step 5: 2-(2,6-dioxo-1-((2-(trimethylsilyl)ethoxy)methyl)piperidin-3-yl)-5-(3-oxo-[1,3′-biazetidin]-1′-yl)isoindoline-1,3-dione

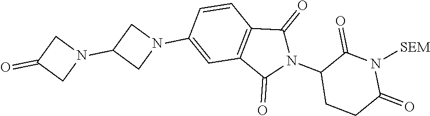 [SMILES: C[Si](C)(C)CCOCN1C(=O)CCC(N2C(=O)c3ccc(N4CC(N5CC(=O)C5)C4)cc3C2=O)C1=O]

To a stirred mixture of 2-(2,6-dioxo-1-((2-(trimethylsilyl)ethoxy)methyl)piperidin-3-yl)-5-(3-hydroxy-[1,3′-biazetidin]-1′-yl)isoindoline-1,3-dione (4.8 g, 9.33 mmol) in CH₂Cl₂ (100 mL) was added DMP (7.9 g, 18.65 mmol) in portions at room temperature. The resulting mixture was stirred for 16 h at room temperature. To the resulting mixture was added water (300 mL) and extracted with CH₂Cl₂ (3×300 mL). The combined organic layers were washed with brine (2×300 mL) and dried over anhydrous Na₂SO₄. After filtration, the filtrate was concentrated under reduced pressure. The residue was purified by silica gel column chromatography, eluted with 40%-70% EtOAc in CH₂Cl₂ to afford crude product. The crude product was further purified by Prep-TLC (CH₂Cl₂/EtOAc=1:3) to afford the product (1.2 g, 25%). ¹H NMR (400 MHz, CDCl₃) δ 7.68 (d, J=8.2 Hz, 1H), 6.82 (d, J=2.2 Hz, 1H), 6.58 (dd, J=8.3, 2.2 Hz, 1H), 5.30 (d, J=15.1 Hz, 2H), 5.02-4.93 (m, 1H), 4.29 (s, 4H), 4.21-4.11 (m, 2H), 4.01-3.90 (m, 3H), 3.70-3.58 (m, 2H), 3.09-2.95 (m, 1H), 2.90-2.73 (m, 2H), 2.18-2.04 (m, 1H), 1.03-0.90 (m, 2H), 0.02 (s, 9H); [M+H₂O+H]⁺=531.4.

### Step 6: N-((1R)-1-(4-(7-(4-(1′-(2-(2,6-dioxo-1-((2-(trimethylsilyl)ethoxy)methyl)piperidin-3-yl)-1,3-dioxoisoindolin-5-yl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

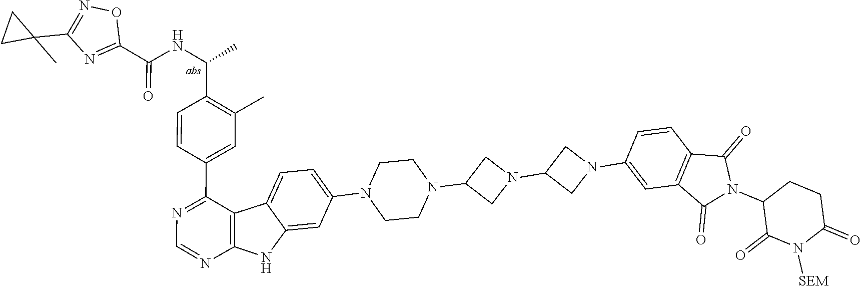 [SMILES: Cc1cc(-c2ncnc3[nH]c4cc(N5CCN(C6CN(C7CN(c8ccc9c(c8)C(=O)N(C8CCC(=O)N(COCC[Si](C)(C)C)C8=O)C9=O)C7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (150 mg, crude), 2-(2,6-dioxo-1-((2-(trimethylsilyl)ethoxy)methyl)piperidin-3-yl)-5-(3-oxo-[1,3′-biazetidin]-1′-yl)isoindoline-1,3-dione (100 mg, 0.195 mmol) and NaOAc (65 mg, 0.798 mmol) in tetrahydrofuran (4 mL) and N,N-dimethylformamide (4 mL) was stirred at room temperature for 5 mins. Then Ti(OiPr)₄ (0.2 mL) was added. The mixture was stirred at room temperature overnight. Then NaBH(OAc)₃ (282 mg, 1.33 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the product (100 mg, crude). [M+2H]²⁺=517.5.

### Step 7: N-((1R)-1-(4-(7-(4-(1′-(2-(2,6-dioxopiperidin-3-yl)-1,3-dioxoisoindolin-5-yl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

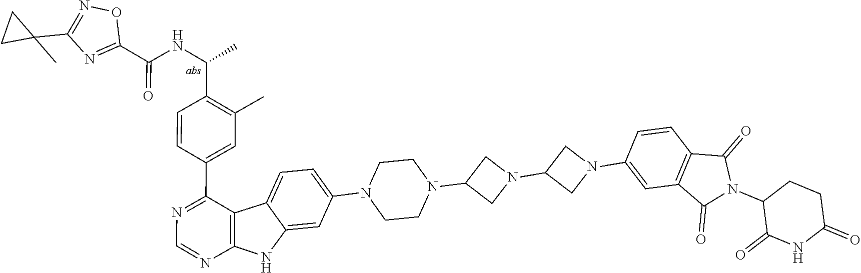 [SMILES: Cc1cc(-c2ncnc3[nH]c4cc(N5CCN(C6CN(C7CN(c8ccc9c(c8)C(=O)N(C8CCC(=O)NC8=O)C9=O)C7)C6)CC5)ccc4c23)ccc1[C@@H](C)NC(=O)c1nc(C2(C)CC2)no1]

To a solution of N-((1R)-1-(4-(7-(4-(1′-(2-(2,6-dioxo-1-((2-(trimethylsilyl)ethoxy)methyl)piperidin-3-yl)-1,3-dioxoisoindolin-5-yl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide (100 mg, 0.097 mmol) in dichloromethane (10 mL), TFA (5 mL) was added. The mixture was stirred at room temperature overnight. The reaction mixture was concentrated in vacuum, and the residue was diluted with MeOH (5 mL). NH₃ (7 M) in MeOH (0.1 mL) was added dropwise and the mixture was stirred at room temperature for 0.5 h. Then the mixture was concentrated in vacuo and the residue was purified by prep-TLC (DCM:MeOH=10:1) to afford desired product (23.54 mg, 28%). ¹H NMR (500 MHz, DMSO) δ 12.18 (s, 1H), 11.07 (s, 1H), 9.86 (d, J=8.0 Hz, 1H), 8.82 (s, 1H), 7.80-7.6 (m, 5H), 6.94-6.90 (m, 2H), 6.79 (s, 1H), 6.66 (d, J=8.0 Hz, 1H), 5.47-5.26 (m, 1H), 5.05 (dd, J=12.0, 4.0 Hz, 1H), 4.05 (t, J=8.0 Hz, 2H), 3.81 (dd, J=8.0, 4.0 Hz, 2H), 3.64 (s, 1H), 3.42 (s, 2H), 3.25 (s, 4H), 2.99 (s, 3H), 2.92-2.83 (m, 1H), 2.62-2.53 (m, 2H), 2.44 (s, 4H), 2.05-1.98 (m, 1H), 1.75 (s, 1H), 1.56 (d, J=8.0 Hz, 3H), 1.49 (s, 3H), 1.23 (s, 2H), 1.21-1.17 (m, 2H), 0.99 (d, J=4.0 Hz, 2H); [M+H]⁺=903.6.

### Example 122: (R)—N-(1-(4-(7-(4-(1′-(4-(2,4-dioxotetrahydropyrimidin-1(2H)-yl)phenyl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: (R)—N-(1-(4-(7-(4-(1′-(4-(2,4-dioxo-3-((2-(trimethylsilyl)ethoxy)methyl)tetrahydropyrimidin-1(2H)-yl)phenyl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

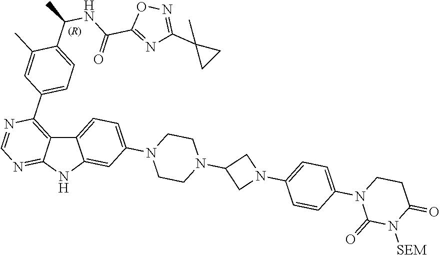

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (0.049 g, 0.09 mmol) and 1-(4-(3-oxo-[1,3′-biazetidin]-1′-yl)phenyl)-3-((2-(trimethylsilyl)ethoxy)methyl)dihydropyrimidine-2,4(1H,3H)-dione (0.06 g, 0.135 mmol) (the compound was synthesised through the similar way of example 121) in 1,2-dichloroethane (5 mL) and HOAc (6 mg) was stirred in a round bottom flask at room temperature for 0.5 hour. To the mixture was added NaBH(OAc)₃ (0.143 g, 0.671 mmol) and stirred in a round bottom flask at room temperature for 3 hours. Then the mixture was evaporated in vacuum to afford the crude product, which was purified by silica gel column chromatography, eluted with 1%-10% MeOH in DCM to afford the product (0.07 g, 80%). [M+H]⁺=965.5.

### Step 2: (R)—N-(1-(4-(7-(4-(1′-(4-(2,4-dioxotetrahydropyrimidin-1(2H)-yl)phenyl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide

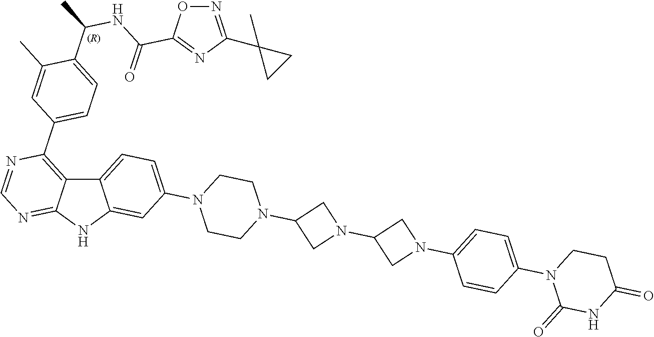

A mixture of (R)—N-(1-(4-(7-(4-(1′-(4-(2,4-dioxo-3-((2-(trimethylsilyl)ethoxy)methyl)tetrahydropyrimidin-1(2H)-yl)phenyl)-[1,3′-biazetidin]-3-yl)piperazin-1-yl)-9H-pyrimido[4,5-b]indol-4-yl)-2-methylphenyl)ethyl)-3-(1-methylcyclopropyl)-1,2,4-oxadiazole-5-carboxamide (0.07 g, 0.073 mmol) and trifluoroacetic acid (5 mL) in dichloromethane (5 mL) was stirred in a round bottom flask at room temperature overnight. The mixture was evaporated in vacuum to afford brown oil. Then MeOH (5 mL) and NH₃/H₂O (2 mL) were added to the residue and the mixture was allowed to stir at 0° C. for 30 min. LCMS showed the reaction was completed. The mixture was evaporated in vacuum to afford the crude product, which was purified by pre-HPLC to afford the product (23.26 mg, 30%). ¹H NMR (500 MHz, DMSO) δ 12.16 (s, 1H), 10.23 (s, 1H), 9.85 (d, J=7.9 Hz, 1H), 8.82 (s, 1H), 7.77 (d, J=8.0 Hz, 1H), 7.73 (s, 1H), 7.70-7.65 (m, 2H), 7.10 (d, J=8.7 Hz, 2H), 6.96-6.86 (m, 2H), 6.42 (d, J=8.8 Hz, 2H), 5.44-5.33 (m, 1H), 3.83 (s, 2H), 3.67 (t, J=6.7 Hz, 2H), 3.58 (d, J=4.3 Hz, 3H), 3.40 (s, 2H), 3.24 (s, 4H), 3.02-2.93 (m, 3H), 2.67 (t, J=6.7 Hz, 2H), 2.50 (s, 3H), 2.43 (s, 4H), 1.56 (d, J=7.0 Hz, 3H), 1.49 (s, 3H), 1.25-1.16 (m, 2H), 1.01-0.94 (m, 2H); [M+H]⁺=835.5.

### Example 123: N-((1R)-1-(4-(7-(4-(2-(1′-(2,6-dioxo-1-((2-(trimethylsilyl)ethoxy)methyl)piperidin-3-yl)-2′-oxospiro[cyclopropane-1,3′-indolin]-5′-yl)ethyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: 3-(5′-bromo-2′-oxospiro[cyclopropane-1,3′-indolin]-1′-yl)-1-((2-(trimethylsilyl)ethoxy)methyl)piperidine-2,6-dione

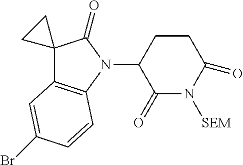 [SMILES: C[Si](C)(C)CCOCN1C(=O)CCC(N2C(=O)C3(CC3)c3cc(Br)ccc32)C1=O]

To a stirred mixture of 3-[5-bromo-2-oxospiro[cyclopropane-1,3-indol]-1-yl]piperidine-2,6-dione (950 mg, 2.721 mmol) and DIEA (8.99 mL, 54.420 mmol) in DMF (45 mL) was added SEMCl (1.93 mL, 10.884 mmol) dropwise at 0° C. under nitrogen atmosphere. The resulting mixture was stirred for 3 h at room temperature. The reaction was quenched with water (300 mL) at room temperature and extracted with EtOAc (2×200 mL). The combined organic layers were washed with brine (3×80 mL) and dried over anhydrous Na₂SO₄. After filtration, the filtrate was concentrated under reduced pressure. The residue was purified by silica gel column chromatography, eluted with EtOAc in petroleum ether (0-20%) to afford the product (848 mg, 65.01%). [M−H]⁻=477.0.

### Step 2: 3-(5′-allyl-2′-oxospiro[cyclopropane-1,3′-indolin]-1′-yl)-1-((2-(trimethylsilyl)ethoxy)methyl)piperidine-2,6-dione

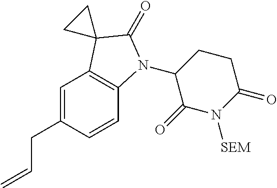 [SMILES: C=CCc1ccc2c(c1)C1(CC1)C(=O)N2C1CCC(=O)N(COCC[Si](C)(C)C)C1=O]

To a stirred solution of 3-(5′-bromo-2′-oxospiro[cyclopropane-1,3′-indolin]-1′-yl)-1-((2-(trimethylsilyl)ethoxy)methyl)piperidine-2,6-dione (850 mg, 1.773 mmol) and tributyl(prop-2-en-1-yl)stannane (0.88 g, 2.659 mmol) in DMF (16 mL) was added Pd(PPh₃)₂Cl₂ (0.19 mL, 0.266 mmol) at room temperature under nitrogen atmosphere. The resulting mixture was stirred for 2 h at 105° C. under nitrogen atmosphere and diluted with water (50 mL). The resulting mixture was extracted with EtOAc (3×100 mL). The combined organic layers were washed with brine (3×50 mL) and dried over anhydrous Na₂SO₄. After filtration, the filtrate was concentrated under reduced pressure. The residue was purified by silica gel column chromatography, eluted with 1˜40% EtOAc in petroleum ether to afford crude product. The crude product was purified by reverse phase flash chromatography with the following conditions: column, C18; mobile phase, MeCN in water (0.1% FA), 50% to 90% gradient in 10 min; detector, UV 254 nm. This afford the product (350 mg, 45%). ¹H NMR (400 MHz, CDCl₃) δ 7.02 (dd, J=8.0, 1.6 Hz, 1H), 6.72 (d, J=1.6 Hz, 1H), 6.62 (d, J=8.0 Hz, 1H), 6.01-5.86 (m, 1H), 5.36-5.21 (m, 3H), 5.14-5.02 (m, 2H), 3.75-3.60 (m, 2H), 3.36 (d, J=6.4 Hz, 2H), 3.10-2.99 (m, 1H), 2.92-2.80 (m, 1H), 2.75-2.62 (m, 1H), 2.23-2.12 (m, 1H), 1.91-1.72 (m, 2H), 1.61-1.57 (m, 2H), 1.04-0.92 (m, 2H), 0.02 (s, 9H); [M+Na]⁺=463.4.

### Step 3: 2-(1′-(2,6-dioxo-1-((2-(trimethylsilyl)ethoxy)methyl)piperidin-3-yl)-2′-oxospiro[cyclopropane-1,3′-indolin]-5′-yl)acetaldehyde

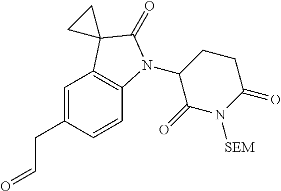 [SMILES: C[Si](C)(C)CCOCN1C(=O)CCC(N2C(=O)C3(CC3)c3cc(CC=O)ccc32)C1=O]

A mixture of 3-(5′-allyl-2′-oxospiro[cyclopropane-1,3′-indolin]-1′-yl)-1-((2-(trimethylsilyl)ethoxy)methyl)piperidine-2,6-dione (100 mg, 0.227 mmol), K₂OsO₄·H₂O (0.83 mg, 0.0023 mmol) and NaIO₄ (194 mg, 0.908 mmol) in dioxane/water (8 mL/2 mL) was stirred at room temperature overnight. Then the mixture was concentrated in vacuo and the residue was purified by silica gel column chromatography (PE:EA=75%:25%) to give the product (140 mg, crude). [M+H]⁺=443.5.

### Step 4: N-((1R)-1-(4-(7-(4-(2-(1′-(2,6-dioxo-1-((2-(trimethylsilyl)ethoxy)methyl)piperidin-3-yl)-2′-oxospiro[cyclopropane-1,3′-indolin]-5′-yl)ethyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

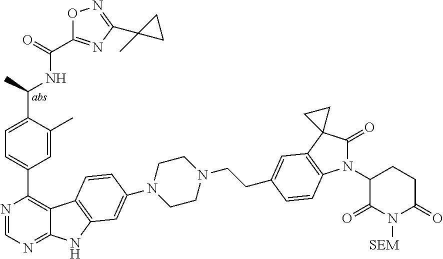 [SMILES: Cc1cc(-c2ncnc3[nH]c4cc(N5CCN(CCc6ccc7c(c6)C6(CC6)C(=O)N7C6CCC(=O)N(COCC[Si](C)(C)C)C6=O)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (169 mg, crude), 2-(1′-(2,6-dioxo-1-((2-(trimethylsilyl)ethoxy)methyl)piperidin-3-yl)-2′-oxospiro[cyclopropane-1,3′-indolin]-5′-yl)acetaldehyde (140 mg, crude) and NaOAc (78 mg, 0.948 mmol) in dichloromethane (4 mL) and MeOH (4 mL) was stirred at room temperature for 5 mins. Then HOAc (0.06 mL) was added. The mixture was stirred at room temperature overnight. Then NaBH(OAc)₃ (334 mg, 1.59 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give the product (42 mg, crude). [M+H]⁺=963.5.

### Step 5: N-((1R)-1-(4-(7-(4-(2-(1′-(2,6-dioxopiperidin-3-yl)-2′-oxospiro[cyclopropane-1,3′-indolin]-5′-yl)ethyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

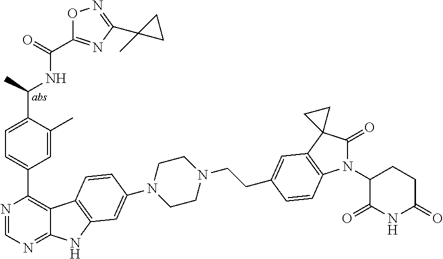 [SMILES: Cc1cc(-c2ncnc3[nH]c4cc(N5CCN(CCc6ccc7c(c6)C6(CC6)C(=O)N7C6CCC(=O)NC6=O)CC5)ccc4c23)ccc1[C@@H](C)NC(=O)c1nc(C2(C)CC2)no1]

To a solution of N-((1R)-1-(4-(7-(4-(2-(1′-(2,6-dioxo-1-((2-(trimethylsilyl)ethoxy)methyl)piperidin-3-yl)-2′-oxospiro[cyclopropane-1,3′-indolin]-5′-yl)ethyl)piperazin-1-yl)-9H-pyrimido[4,5-b]indol-4-yl)-2-methylphenyl)ethyl)-3-(1-methylcyclopropyl)-1,2,4-oxadiazole-5-carboxamide (42 mg, 0.045 mmol) in dichloromethane (3 mL), TFA (2.5 mL) was added. The mixture was stirred at room temperature overnight. The reaction mixture was concentrated in vacuum, and the residue was diluted with MeOH (3 mL). NH₃ (7 M) in MeOH (1.5 mL) was added dropwise, and the mixture was stirred at room temperature for 40 mins. Then the mixture was concentrated in vacuo, and the residue was purified by prep-TLC (DCM:MeOH=10:1) to afford desired product (23.54 mg, 84%). ¹H NMR (500 MHz, DMSO) δ 12.17 (s, 1H), 11.08 (s, 1H), 9.86 (d, J=8.0 Hz, 2H), 8.83 (s, 1H), 7.82-7.58 (m, 4H), 7.09 (s, 1H), 6.96 (s, 4H), 5.42-5.27 (m, 2H), 3.28-3.14 (m, 6H), 2.95-2.83 (m, 2H), 2.80-2.53 (m, 8H), 1.98-1.91 (m, 1H), 1.63 (s, 2H), 1.59-1.52 (m, 5H), 1.30-1.17 (m, 8H), 0.99 (d, J=2.0 Hz, 2H); [M+H]⁺=833.6.

### Example 124: N-((1R)-1-(4-(7-(4-(2-(1′-(2,6-dioxopiperidin-3-yl)-2′-oxospiro[cyclopropane-1,3′-indolin]-4′-yl)ethyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

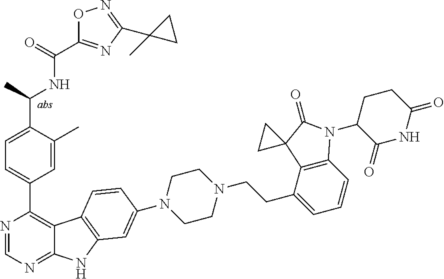 [SMILES: Cc1cc(-c2ncnc3[nH]c4cc(N5CCN(CCc6cccc7c6C6(CC6)C(=O)N7C6CCC(=O)NC6=O)CC5)ccc4c23)ccc1[C@@H](C)NC(=O)c1nc(C2(C)CC2)no1]

The titled compound was synthesized in the procedures similar to Example 123. ¹H NMR (500 MHz, DMSO) δ 12.17 (s, 1H), 11.08 (s, 1H), 9.86 (d, J=8.0 Hz, 1H), 8.82 (s, 1H), 7.87-7.60 (m, 4H), 7.17 (s, 1H), 7.02-6.75 (m, 4H), 5.45-5.27 (m, 2H), 3.28 (s, 5H), 2.87 (t, J=10.0 Hz, 1H), 2.67-2.60 (m, 4H), 2.59-2.54 (m, 3H), 2.01-1.93 (m, 3H), 1.56 (d, J=5.0 Hz, 4H), 1.49 (s, 5H), 1.27-1.18 (m, 7H), 0.99 (d, J=2.0 Hz, 2H); [M+H]⁺=833.7.

### Example 125: (R)—N-(1-(4-(7-(4-(3-(4-(2,4-dioxotetrahydropyrimidin-1(2H)-yl)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: 3-(4-(4,4,5,5-tetramethyl-1,3,2-dioxaborolan-2-yl)phenyl)cyclobutan-1-one

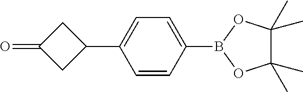 [SMILES: CC1(C)OB(c2ccc(C3CC(=O)C3)cc2)OC1(C)C]

A mixture of 3-(4-bromophenyl)cyclobutan-1-one (4.5 g, 0.02 mol), 4,4,4′,4′,5,5,5′,5′-octamethyl-2,2′-bi(1,3,2-dioxaborolane) (7.62 g, 0.03 mol), Pd(dppf)Cl₂ (0.731 g, 0.001 mol) and KOAc (3.92 g, 0.04 mol) in dioxane (200 mL) was stirred for 16 h at 100° C. under nitrogen atmosphere overnight. The mixture was allowed to cool down to room temperature and concentrated under reduced pressure. The residue was purified by silica gel column chromatography, eluted with PE/EtOAc (100%:0% to 80%:20%) to afford the product (3.9 g, 71%). [M+H]⁺=273.4.

### Step 2: (4-(3-oxocyclobutyl)phenyl)boronic acid

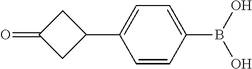 [SMILES: O=C1CC(c2ccc(B(O)O)cc2)C1]

A solution of 3-(4-(4,4,5,5-tetramethyl-1,3,2-dioxaborolan-2-yl)phenyl)cyclobutan-1-one (3.9 g, 0.0143 mol) and NaIO₄ (6.13 g, 0.0286 mol) in THF/water (40 mL/10 mL) was stirred at rt for 20 mins. Then HCl (2 M, 10 mL) was added. The mixture was stirred at room temperature overnight. The mixture was extracted with EA, washed with brine, dried over Na₂SO₄, and concentrated in vacuo. The residue was purified by silica gel column chromatography, eluted with DCM/MeOH (100%:0% to 90%:10%) to afford the product (1.6 g, 58%). [M+H]⁺=191.4.

### Step 3: 1-(4-(3-oxocyclobutyl)phenyl)-3-((2-(trimethylsilyl)ethoxy)methyl)pyrimidine-2,4(1H,3H)-dione

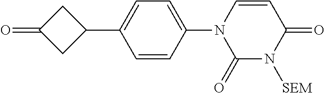 [SMILES: C[Si](C)(C)CCOCn1c(=O)ccn(-c2ccc(C3CC(=O)C3)cc2)c1=O]

A mixture of (4-(3-oxocyclobutyl)phenyl)boronic acid (1.6 g, 8.42 mmol), and 3-((2-(trimethylsilyl)ethoxy)methyl)pyrimidine-2,4(1H,3H)-dione (2 g, 8.42 mmol), Cu(OAc)₂ (4.57 g, 25.26 mmol), pyridine (3.32 g, 42.9 mmol) and 4 A MS in DMA (30 mL) was stirred at 80° C. for 2 h under oxygen. The mixture was allowed to cool down to room temperature. The resulting mixture was extracted with EA (100 mL*3), washed with brine (150 mL), dried over Na₂SO₄, and concentrated under reduced pressure. The residue was purified by silica gel column chromatography, eluted with PE/EA (100%:0%-0%:100%) to afford the product (700 mg, 21%). [M+H]⁺=387.5.

### Step 4: 1-(4-(3-oxocyclobutyl)phenyl)-3-((2-(trimethylsilyl)ethoxy)methyl)dihydropyrimidine-2,4(1H,3H)-dione

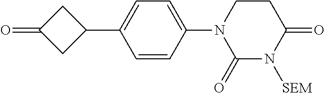 [SMILES: C[Si](C)(C)CCOCN1C(=O)CCN(c2ccc(C3CC(=O)C3)cc2)C1=O]

To a solution of 1-(4-(3-oxocyclobutyl)phenyl)-3-((2-(trimethylsilyl)ethoxy)methyl)pyrimidine-2,4(1H,3H)-dione (700 mg, 1.81 mmol) and Pd/C (350 mg) in THF (20 mL), HCl (2 M) was added. The mixture was stirred at room temperature overnight under 4 atm of hydrogen. The mixture was filtered, and the filtration was concentrated in vacuo to give crude product (400 mg, crude). [M+H]⁺=389.5.

### Step 5: 1-(4-(3-oxocyclobutyl)phenyl)dihydropyrimidine-2,4(1H,3H)-dione

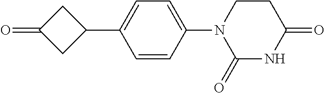 [SMILES: O=C1CC(c2ccc(N3CCC(=O)NC3=O)cc2)C1]

To a suspension of 1-(4-(3-oxocyclobutyl)phenyl)-3-((2-(trimethylsilyl)ethoxy)methyl)dihydropyrimidine-2,4(1H,3H)-dione (400 mg, 1.03 mmol) in DCM, TFA (10 mL) was added. The mixture was stirred at room temperature for 6 h. The mixture was concentrated in vacuo. The residue was diluted with DCM (1 mL), and NH₃·H₂O (3 mL) was added. The mixture was stirred at room temperature for 2 h. The mixture was concentrated in vacuo and the residue was purified by silica gel column chromatography, eluted with DCM/MeOH (100%:0% to 95%:5%) to afford the product (120 mg, 46%). [M+H]⁺=259.5.

### Step 6: (R)—N-(1-(4-(7-(4-(3-(4-(2,4-dioxotetrahydropyrimidin-1(2H)-yl)phenyl)cyclobutyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

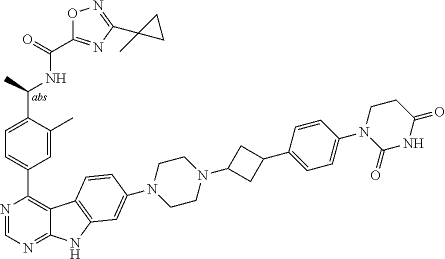 [SMILES: Cc1cc(-c2ncnc3[nH]c4cc(N5CCN(C6CC(c7ccc(N8CCC(=O)NC8=O)cc7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (150 mg, crude), 1-(4-(3-oxocyclobutyl)phenyl)dihydropyrimidine-2,4(1H,3H)-dione (72 mg, 0.280 mmol) and NaOAc (69 mg, 0.84 mmol) in dichloromethane (5 mL) and MeOH (5 mL) was stirred at room temperature for 5 mins. Then HOAc (0.06 mL) was added. The mixture was stirred at room temperature overnight. Then NaBH(OAc)₃ (297 mg, 1.4 mmol) was added and stirred at room temperature for 2 hours. The reaction mixture was concentrated in vacuum, and the residue was purified by silica gel column chromatography (DCM:MeOH=100%: 0%˜90%: 10% gradient elution) to give crude product, which was further purified by prep-TLC (DCM:MeOH=10:1) to afford desired product (7.58 mg, 3.4%). ¹H NMR (500 MHz, DMSO) δ 12.18 (s, 1H), 10.34 (s, 1H), 9.86 (d, J=10.0 Hz, 1H), 8.83 (s, 1H), 7.92-7.64 (m, 4H), 7.35-7.20 (m, 4H), 6.95 (s, 2H), 5.51-5.31 (m, 1H), 3.76 (t, J=5.0 Hz, 2H), 3.31-3.09 (m, 9H), 2.82-2.74 (m, 1H), 2.70 (t, J=5.0 Hz, 2H), 2.58-2.52 (m, 2H), 2.48-2.42 (m, 2H), 1.95-1.85 (m, 1H), 1.56 (d, J=10.0 Hz, 3H), 1.49 (s, 3H), 1.29-1.15 (m, 6H), 0.99 (s, 2H); [M+H]⁺=779.6.

### Example 126: (R)—N-(1-(4-(7-(4-((1-(4-(2,4-dioxotetrahydropyrimidin-1(2H)-yl)phenyl)azetidin-3-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

### Step 1: 4-(2,4-dioxo-3-((2-(trimethylsilyl)ethoxy)methyl)tetrahydropyrimidin-1(2H)-yl)phenyl trifluoromethanesulfonate

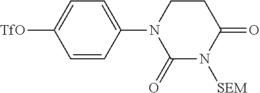 [SMILES: C[Si](C)(C)CCOCN1C(=O)CCN(c2ccc(OS(=O)(=O)C(F)(F)F)cc2)C1=O]

To a solution of 1-(4-hydroxyphenyl)-3-((2-(trimethylsilyl)ethoxy)methyl)dihydropyrimidine-2,4(1H,3H)-dione (500 mg, 1.49 mmol) in DCM, pyridine (235 mg, 2.98 mmol) was added. Then Tf₂O (630 mg, 2.24 mmol) was added dropwise between 0° C. and 5° C. The mixture was stirred from 0° C. to room temperature and stirred at room temperature for 3 h. The resulting mixture was concentrated under reduced pressure. The residue was purified by silica gel column chromatography, eluted with PE/EtOAc (100%:0% to 50%:50%) to afford the product (644 mg, 92%). [M+H]⁺=469.5.

### Step 2: 1-(4-(3-(hydroxymethyl)azetidin-1-yl)phenyl)-3-((2-(trimethylsilyl)ethoxy)methyl)dihydropyrimidine-2,4(1H,3H)-dione

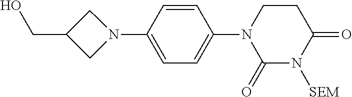 [SMILES: C[Si](C)(C)CCOCN1C(=O)CCN(c2ccc(N3CC(CO)C3)cc2)C1=O]

A mixture of 4-(2,4-dioxo-3-((2-(trimethylsilyl)ethoxy)methyl)tetrahydropyrimidin-1(2H)-yl)phenyl trifluoromethanesulfonate (100 mg, 0.213 mmol), azetidin-3-ylmethanol hydrochloride (39 mg, 0.320 mmol), Pd₂(dba)₃ (19.5 mg, 0.0213 mmol), Ruphos (20 mg, 0.0426 mmol) and Cs₂CO₃ (138 mg, 0.426 mmol) in dioxane (10 mL) was stirred at 100° C. overnight under nitrogen. After the reaction was completed determined by LCMS, the mixture was allowed to cooled down to room temperature, and concentrated in vacuo. The residue was purified by silica gel column chromatography, eluted with PE/EA (100%:0% to 50%:50%) to afford the product (67 mg, 78%). [M+H]⁺=407.4.

### Step 3: 1-(4-(2,4-dioxo-3-((2-(trimethylsilyl)ethoxy)methyl)tetrahydropyrimidin-1(2H)-yl)phenyl)azetidine-3-carbaldehyde

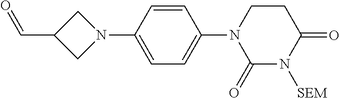 [SMILES: C[Si](C)(C)CCOCN1C(=O)CCN(c2ccc(N3CC(C=O)C3)cc2)C1=O]

To a solution of 1-(4-(3-(hydroxymethyl)azetidin-1-yl)phenyl)-3-((2-(trimethylsilyl)ethoxy)methyl)dihydropyrimidine-2,4(1H,3H)-dione (132 mg, 0.325 mmol) in DMSO (10 mL), IBX (114 mg, 0.406 mmol) was added. The mixture was stirred at room temperature overnight. The resulting mixture was extracted with EA (50 mL*3), washed with brine (100 mL), dried over Na₂SO₄, and concentrated under reduced pressure to afford the product (140 mg, crude). [M+H]⁺=405.5.

### Step 4: (R)—N-(1-(4-(7-(4-((1-(4-(2,4-dioxo-3-((2-(trimethylsilyl)ethoxy)methyl)tetrahydropyrimidin-1(2H)-yl)phenyl)azetidin-3-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

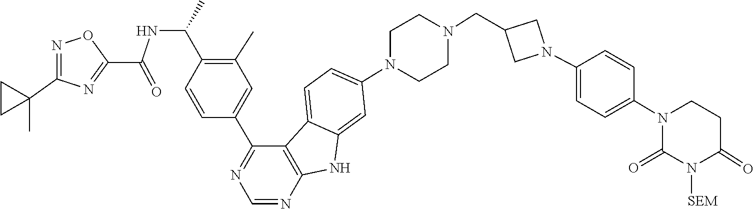 [SMILES: Cc1cc(-c2ncnc3[nH]c4cc(N5CCN(CC6CN(c7ccc(N8CCC(=O)N(COCC[Si](C)(C)C)C8=O)cc7)C6)CC5)ccc4c23)ccc1[C@@H](C)NC(=O)c1nc(C2(C)CC2)no1]

A mixture of (R)—N-(1-(2-methyl-4-(7-(piperazin-1-yl)-9H-pyrimido[4,5-b]indol-4-yl)phenyl)ethyl)-3-(1-methylcyclopropyl)-1,2,4-oxadiazole-5-carboxamide (200 mg, crude), 1-(4-(2,4-dioxo-3-((2-(trimethylsilyl)ethoxy)methyl)tetrahydropyrimidin-1(2H)-yl)phenyl)azetidine-3-carbaldehyde (140 mg, crude) and NaOAc (153 mg, 1.865 mmol) in dichloromethane (8 mL) and MeOH (8 mL) was stirred at room temperature for 5 mins. Then HOAc (0.06 mL) was added. The mixture was stirred at room temperature overnight. Then NaBH(OAc)₃ (395 mg, 1.865 mmol) was added and stirred at room temperature for 2 hours. The reaction mixture was concentrated in vacuum, and the residue was purified by silica gel column chromatography (DCM:MeOH=100%:0%˜90%:10% gradient elution) to give the product (60 mg, crude). [M+H]⁺=924.6.

### Step 5: (R)—N-(1-(4-(7-(4-((1-(4-(2,4-dioxotetrahydropyrimidin-1(2H)-yl)phenyl)azetidin-3-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide

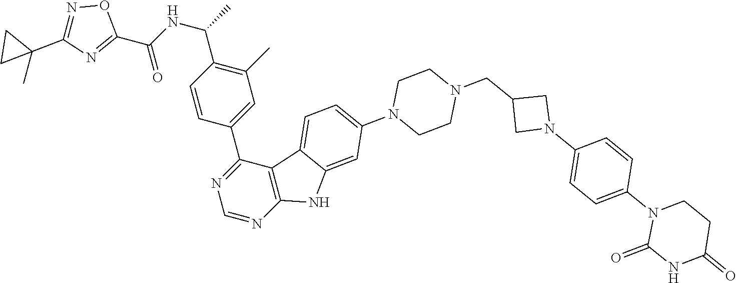 [SMILES: Cc1cc(-c2ncnc3[nH]c4cc(N5CCN(CC6CN(c7ccc(N8CCC(=O)NC8=O)cc7)C6)CC5)ccc4c23)ccc1[C@@H](C)NC(=O)c1nc(C2(C)CC2)no1]

To a solution of (R)—N-(1-(4-(7-(4-((1-(4-(2,4-dioxo-3-((2-(trimethylsilyl)ethoxy)methyl)tetrahydropyrimidin-1(2H)-yl)phenyl)azetidin-3-yl)methyl)piperazin-1-yl)-9H-pyrimido[4,5-b]indol-4-yl)-2-methylphenyl)ethyl)-3-(1-methylcyclopropyl)-1,2,4-oxadiazole-5-carboxamide (60 mg, 0.045 mmol) in dichloromethane (5 mL), TFA (5 mL) was added. The mixture was stirred at room temperature overnight. The reaction mixture was concentrated in vacuum, and the residue was diluted with MeOH (5 mL). NH₃ (7 M) in MeOH (0.1 mL) was added dropwise, and the mixture was stirred at room temperature for 0.5 h. Then the mixture was concentrated in vacuo, the residue was purified by prep-TLC (DCM:MeOH=10:1) to afford desired product (16.62 mg, 33%). ¹H NMR (500 MHz, DMSO) δ 12.17 (s, 1H), 10.23 (s, 1H), 9.86 (d, J=10.0 Hz, 1H), 8.82 (d, J=2.0 Hz, 1H), 7.95-7.57 (m, 4H), 7.10 (d, J=10.0 Hz, 2H), 6.93 (s, 2H), 6.54-6.31 (m, 2H), 5.53-5.24 (m, 1H), 3.95 (t, J=7.5 Hz, 2H), 3.67 (t, J=5.0 Hz, 2H), 3.48 (t, J=5.0 Hz, 2H), 3.25 (s, 4H), 3.04-2.92 (m, 1H), 2.72-2.62 (m, 4H), 2.57 (s, 3H), 2.48-2.32 (m, 3H), 1.57 (d, J=5.0 Hz, 2H), 1.49 (s, 3H), 1.25-1.19 (m, 3H), 0.99 (s, 2H); [M+H]⁺=794.6.

### Example 129: 3-(tert-butyl)-N-((3R,6S)-1-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)-6-methylpiperidin-3-yl)-1,2,4-oxadiazole-5-carboxamide

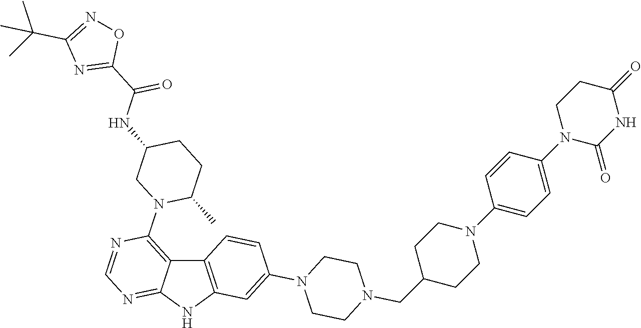 [SMILES: C[C@H]1CC[C@@H](NC(=O)c2nc(C(C)(C)C)no2)CN1c1ncnc2[nH]c3cc(N4CCN(CC5CCN(c6ccc(N7CCC(=O)NC7=O)cc6)CC5)CC4)ccc3c12]

The titled compound was synthesized in the procedures similar to Example 126. ¹H NMR (500 MHz, DMSO) δ 11.84 (s, 1H), 10.25 (s, 1H), 8.38 (s, 1H), 7.69 (d, J=10.0 Hz, 1H), 7.13 (d, J=10.0 Hz, 2H), 6.99 (d, J=10.0 Hz, 1H), 6.95-6.85 (m, 3H), 4.72 (s, 1H), 4.28 (d, J=5.0 Hz, 1H), 3.90 (d, J=10.0 Hz, 1H), 3.74-3.63 (m, 4H), 3.23 (s, 4H), 2.76-2.63 (m, 4H), 2.56 (s, 4H), 2.24 (d, J=5.0 Hz, 2H), 2.19-2.03 (m, 1H), 1.89-1.68 (m, 6H), 1.36 (s, 9H), 1.24 (d, J=5.0 Hz, 2H), 1.17 (d, J=5.0 Hz, 2H); [M+H]⁺=803.6.

### Example 130: (R)-3-(tert-butyl)-N-(1-(1-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)piperidin-4-yl)ethyl)-1,2,4-oxadiazole-5-carboxamide

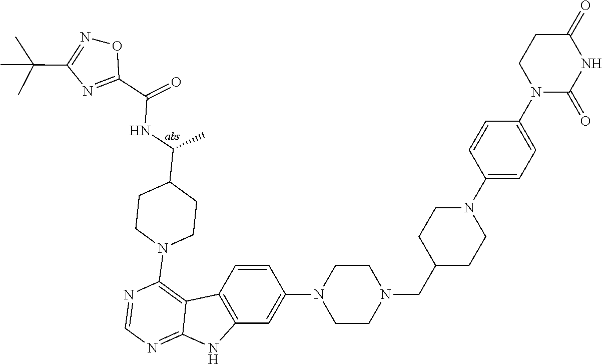 [SMILES: C[C@@H](NC(=O)c1nc(C(C)(C)C)no1)C1CCN(c2ncnc3[nH]c4cc(N5CCN(CC6CCN(c7ccc(N8CCC(=O)NC8=O)cc7)CC6)CC5)ccc4c23)CC1]

The titled compound was synthesized in the procedures similar to Example 126. ¹H NMR (500 MHz, DMSO) δ 11.95 (s, 1H), 11.82 (s, 1H), 10.26 (s, 1H), 9.25 (d, J=10.0 Hz, 1H), 8.37 (s, 1H), 7.53 (d, J=10.0 Hz, 1H), 7.13 (d, J=10.0 Hz, 2H), 7.01 (d, J=10.0 Hz, 1H), 6.93 (d, J=10.0 Hz, 3H), 4.25 (s, 2H), 3.96-3.87 (m, 1H), 3.69 (t, J=5.0 Hz, 4H), 3.22 (s, 4H), 3.02-2.83 (m, 2H), 2.74-2.62 (m, 4H), 2.54 (s, 3H), 2.24 (s, 2H), 1.91-1.87 (m, 1H), 1.86-1.77 (m, 3H), 1.73 (s, 1H), 1.50-1.39 (m, 2H), 1.36 (s, 9H), 1.23 (d, J=5.0 Hz, 4H); [M+H]⁺=817.7.

### Example 131: 3-(tert-butyl)-N-(2-(1-(7-(4-((1-(4-(2,4-dioxotetrahydropyrimidin-1(2H)-yl)phenyl)piperidin-4-yl)methyl)piperazin-1-yl)-9H-pyrimido[4,5-b]indol-4-yl)piperidin-4-yl)propan-2-yl)-1,2,4-oxadiazole-5-carboxamide

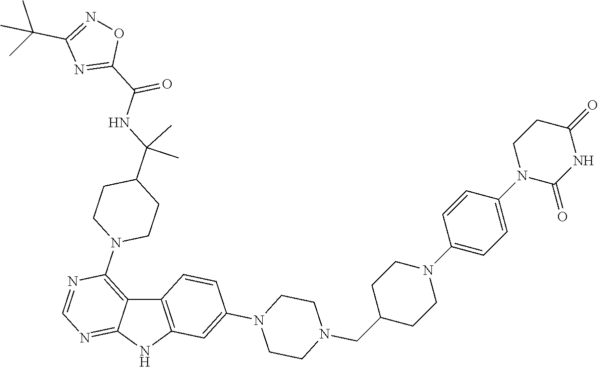 [SMILES: CC(C)(C)c1noc(C(=O)NC(C)(C)C2CCN(c3ncnc4[nH]c5cc(N6CCN(CC7CCN(c8ccc(N9CCC(=O)NC9=O)cc8)CC7)CC6)ccc5c34)CC2)n1]

The titled compound was synthesized in the procedures similar to Example 126. ¹H NMR (500 MHz, DMSO) δ 12.00-11.77 (m, 1H), 10.25 (s, 1H), 8.54 (s, 1H), 8.36 (s, 1H), 7.56 (s, 1H), 7.18-7.10 (m, 2H), 7.01 (s, 1H), 6.93 (s, 3H), 4.34 (d, J=10.0 Hz, 1H), 3.69 (d, J=5.0 Hz, 4H), 3.21 (s, 4H), 2.92 (t, J=10.0 Hz, 2H), 2.68 (s, 4H), 2.60-2.53 (m, 3H), 2.37 (d, J=10.0 Hz, 1H), 2.24 (s, 1H), 1.94-1.88 (m, 1H), 1.88-1.63 (m, 5H), 1.50 (d, J=10.0 Hz, 2H), 1.38 (s, 5H), 1.37-1.32 (m, 9H), 1.23 (s, 3H), 1.12-1.09 (m, 1H); [M+H]⁺=831.6.

### Example 127 and 128: 3-(tert-butyl)-N—((R)-1-(4-(7-(4-((1S,3s)-3-(4-(((R)-2,6-dioxopiperidin-3-yl)oxy)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide and 3-(tert-butyl)-N—((R)-1-(4-(7-(4-((1R,3s)-3-(4-(((S)-2,6-dioxopiperidin-3-yl)oxy)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide

### Step 1: 2-(4-bromophenyl)-5,8-dioxaspiro[3.4]octane

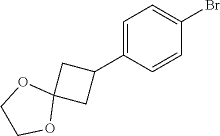 [SMILES: Brc1ccc(C2CC3(C2)OCCO3)cc1]

A solution of 3-(4-bromophenyl)cyclobutan-1-one (10.0 g, 44.43 mmol), 2-dihydroxyethane (9.9 mL, 177.71 mmol) and 4-methylbenzene-1-sulfonic acid hydrate (845 mg, 4.44 mmol) in toluene (200 mL) was stirred for 16 h at 110° C. The mixture was allowed to cool down to room temperature. The resulting mixture was concentrated under reduced pressure. To the resulting mixture was added water (200 mL) and extracted with EtOAc (3×200 mL). The combined organic layers were washed with brine (200 mL) and dried over anhydrous Na₂SO₄. After filtration, the filtrate was concentrated under reduced pressure. The residue was purified by silica gel column chromatography, eluted with 10%-20% EtOAc in petroleum ether to afford the product (11.5 g, 96%). [M+H]⁺=269.0.

### Step 2: 2-(4-(5,8-dioxaspiro[3.4]octan-2-yl)phenyl)-4,4,5,5-tetramethyl-1,3,2-dioxaborolane

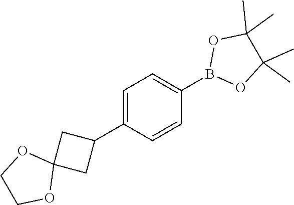 [SMILES: CC1(C)OB(c2ccc(C3CC4(C3)OCCO4)cc2)OC1(C)C]

To a stirred mixture of 2-(4-bromophenyl)-5,8-dioxaspiro[3.4]octane (9.0 g, 33.44 mmol) and 4,4,5,5-tetramethyl-2-(tetramethyl-1,3,2-dioxaborolan-2-yl)-1,3,2-dioxaborolane (12.7 g, 50.16 mmol) in dioxane (180 mL) were added KOAc (6.5 g, 66.88 mmol) and Pd(dppf)Cl₂ (2.4 g, 3.34 mmol) at room temperature under nitrogen atmosphere. The resulting mixture was stirred for 16 h at 80° C. under nitrogen atmosphere. The mixture was allowed to cool down to room temperature and concentrated under reduced pressure. The residue was purified by silica gel column chromatography, eluted with 5%-15% EtOAc in petroleum ether to afford the product (10.2 g, 96%). [M+H]⁺=317.2.

### Step 3: 4-(5,8-dioxaspiro[3.4]octan-2-yl)phenol

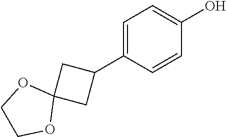 [SMILES: Oc1ccc(C2CC3(C2)OCCO3)cc1]

To a stirred mixture of 2-(4-(5,8-dioxaspiro[3.4]octan-2-yl)phenyl)-4,4,5,5-tetramethyl-1,3,2-dioxaborolane (10.2 g, 32.26 mmol) in THF (200 mL) were added NaOH (aq.) (3M, 20 mL) and H₂O₂ (aq.) (30%, 40 mL) dropwise at 0° C. The resulting mixture was stirred for 4 h at room temperature. The reaction was quenched by the addition of sat. Na₂S₂O₃ (aq.) (80 mL) at 0° C. The resulting mixture was extracted with EtOAc (3×200 mL). The combined organic layers were washed with brine (3×200 mL) and dried over anhydrous Na₂SO₄. After filtration, the filtrate was concentrated under reduced pressure. The residue was purified by silica gel column chromatography, eluted with 10%-30% EtOAc in petroleum ether to afford the product (5.9 g, 88%). [M+H]⁺=207.0.

### Step 4: 3-(4-(5,8-dioxaspiro[3.4]octan-2-yl)phenoxv)piperidine-2,6-dione

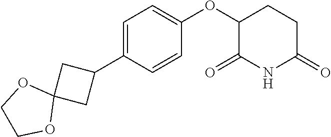 [SMILES: O=C1CCC(Oc2ccc(C3CC4(C3)OCCO4)cc2)C(=O)N1]

To a stirred mixture of 4-(5,8-dioxaspiro[3.4]octan-2-yl)phenol (4.0 g, 19.40 mmol) and 3-bromopiperidine bromopiperidine-2,6-dione (5.6 g, 29.09 mmol) in ACN (80 mL) was added Cs₂CO₃ (12.6 g, 38.79 mmol) at room temperature. The resulting mixture was stirred for 16 h at 60° C. The mixture was allowed to cool down to room temperature and concentrated under reduced pressure. The residue was purified by silica gel column chromatography, eluted with 30%-50% EtOAc in petroleum ether to afford the product (2.3 g, 37%). [M+Na]⁺=340.2.

### Step 5: 3-(4-(3-oxocyclobutyl)phenoxy)piperidine-2,6-dione

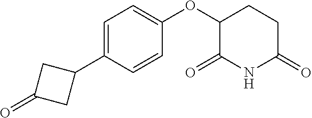 [SMILES: O=C1CC(c2ccc(OC3CCC(=O)NC3=O)cc2)C1]

A mixture of 3-(4-(5,8-dioxaspiro[3.4]octan-2-yl)phenoxy)piperidine-2,6-dione (3.1 g, 9.77 mmol) in THF (30 mL) and HCl (aq.) (2 M, 30 mL) was stirred for 1 h at 55° C. The mixture was allowed to cool down to room temperature. To the resulting mixture was add water (200 mL) and extracted with EtOAc (3×200 mL). The combined organic layers were washed with brine (3×100 mL) and dried over anhydrous Na₂SO₄. After filtration, the filtrate was concentrated under reduced pressure. The residue was purified by silica gel column chromatography, eluted with 30%-50% EtOAc in petroleum ether to afford the product (2.0 g, 75%). [M+H]⁺=274.1.

### Step 6: benzyl 4-(4-chloro-2-methoxyphenyl)piperazine-1-carboxylate

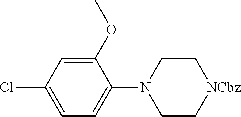 [SMILES: COc1cc(Cl)ccc1N1CCN(C(=O)OCc2ccccc2)CC1]

To a solution of 1-bromo-4-chloro-2-methoxybenzene (50.00 g, 225.754 mmol) and benzyl piperazine-1-carboxylate (59.67 g, 270.905 mmol) in toluene (1000.00 mL) were added BINAP (28.11 g, 45.151 mmol), Pd₂(dba)₃ (20.67 g, 22.575 mmol) and t-BuONa (43.39 g, 451.508 mmol). After stirring for 2 h at 95° C. under nitrogen atmosphere, the resulting mixture was filtered. The filter cake was washed with EA (3×200 mL). The filtrate was concentrated under reduced pressure. The residue was purified by silica gel column chromatography, eluted with PE/EtOAc (20:1 to 3:1) to afford the product (50 g, 61%). [M+H]⁺=361.1.

### Step 7: 1-(4-chloro-2-methoxy-5-nitrophenyl)piperazine

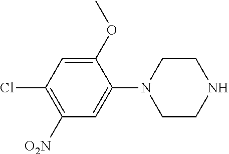 [SMILES: COc1cc(Cl)c([N+](=O)[O-])cc1N1CCNCC1]

To a stirred solution of benzyl 4-(4-chloro-2-methoxyphenyl)piperazine-1-carboxylate (40.00 g, 110.852 mmol) in conc. H₂SO₄ (200.00 mL, 3752.116 mmol) was added guanidine nitrate (16.24 g, 133.023 mmol) in portions at 0° C. under nitrogen atmosphere. The resulting mixture was stirred for 2 h at 0° C. under nitrogen atmosphere. The reaction was quenched by the addition of sat. Na₂CO₃ (aq.) at 0° C. The resulting mixture was extracted with EtOAc (6×500 mL) and 10% CH₃OH in DCM (3×500 mL). The combined organic layers were washed with brine (100 mL) and dried over anhydrous Na₂SO₄. After filtration, the filtrate was concentrated under reduced pressure to give the product (30 g, crude). [M+H]⁺=272.1.

### Step 8: benzyl 4-(4-chloro-2-methoxy-5-nitrophenyl)piperazine-1-carboxylate

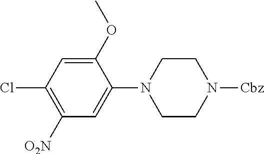 [SMILES: COc1cc(Cl)c([N+](=O)[O-])cc1N1CCN(C(=O)OCc2ccccc2)CC1]

To a stirred solution of 1-(4-chloro-2-methoxy-5-nitrophenyl)piperazine (37.00 g, 136.180 mmol) and DIEA (71.16 mL, 408.540 mmol) in DCM (400.00 mL) was added Cbz-Cl (46.46 g, 272.360 mmol) dropwise at 0° C. under nitrogen atmosphere. The resulting mixture was stirred for 16 h at room temperature and concentrated under reduced pressure. The residue was purified by silica gel column chromatography, eluted with PE/EtOAc (15:1 to 3:1) to afford the product (26.5 g, 48%). [M+H]⁺=406.1.

### Step 9: benzyl 4-(4-(1-cyano-2-ethoxy-2-oxoethyl)-2-methoxy-5-nitrophenyl)piperazine-1-carboxylate

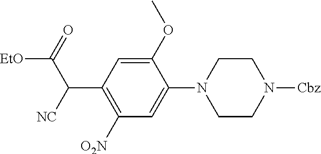 [SMILES: CCOC(=O)C(C#N)c1cc(OC)c(N2CCN(C(=O)OCc3ccccc3)CC2)cc1[N+](=O)[O-]]

To a stirred mixture of Cs₂CO₃ (63.82 g, 195.890 mmol) in DMF (500.00 mL) was added ethyl cyanoacetate (8.86 g, 78.356 mmol) dropwise at 0° C. under nitrogen atmosphere. The resulting mixture was stirred for 0.5 h at 0° C. To the above mixture was added a solution of benzyl 4-(4-chloro-2-methoxy-5-nitrophenyl)piperazine-1-carboxylate (26.50 g, 65.297 mmol) in DMF (30 mL) dropwise at 0° C. The resulting mixture was stirred for additional 16 h at 80° C. The resulting mixture was filtered and the filter cake was washed with EtOAc (3×500 mL). The solution was acidified to pH 5-6 with HCl (aq., 1 M). The resulting solution was extracted with EtOAc (3×500 mL). The combined organic layers were washed with brine (500 mL) and dried over anhydrous Na₂SO₄. After filtration, the filtrate was concentrated under reduced pressure. The residue was purified by silica gel column chromatography, eluted with PE/EA (10:1 to 1:1) to afford the product (28.2 g, 90%). [M+H]⁺=483.1.

### Step 10: ethyl 2-amino-6-(4-((benzyloxy)carbonyl)piperazin-1-yl)-5-methoxy-1H-indole-3-carboxylate

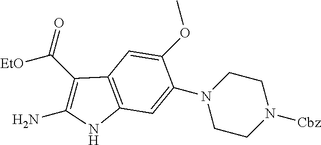 [SMILES: CCOC(=O)c1c(N)[nH]c2cc(N3CCN(C(=O)OCc4ccccc4)CC3)c(OC)cc12]

To a stirred solution of benzyl 4-(4-(1-cyano-2-ethoxy-2-oxoethyl)-2-methoxy-5-nitrophenyl)piperazine-1-carboxylate (28.20 g, 58.446 mmol) in AcOH (282.00 mL) was added Fe (22.85 g, 0.409 mmol) at room temperature. The resulting mixture was stirred for 16 h at room temperature. The resulting mixture was concentrated under reduced pressure. The residue was purified by silica gel column chromatography, eluted with CH₂Cl₂/MeOH (20:1) to afford the product (24.3 g, 92%). [M+H]⁺=453.1.

### Step 11: benzyl 4-(6-methoxy-4-oxo-4,9-dihydro-3H-pyrimido[4,5-b]indol-7-yl)piperazine-1-carboxylate

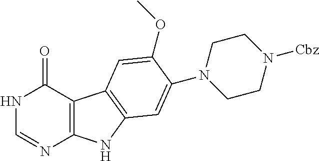 [SMILES: COc1cc2c(cc1N1CCN(C(=O)OCc3ccccc3)CC1)[nH]c1nc[nH]c(=O)c12]

To a stirred solution of ethyl 2-amino-6-(4-((benzyloxy)carbonyl)piperazin-1-yl)-5-methoxy-1H-indole-3-carboxylate (33.10 g, 73.147 mmol) in 2-methoxyethan-1-ol (330 mL) was added acetic acid methanimidamide (76.15 g, 731.470 mmol) in portions at room temperature. The resulting mixture was stirred for 16 h at 125° C. under nitrogen atmosphere. The mixture was allowed to cool down to room temperature and diluted with water (800 mL). The precipitated solid was collected by filtration and washed with water (2×50 mL). The resulting solid was dried under infrared light to afford the product (28 g, 88%). [M+H]⁺=434.1.

### Step 12: benzyl 4-(4-chloro-6-methoxy-9H-pyrimido[4,5-b]indol-7-yl)piperazine-1-carboxylate

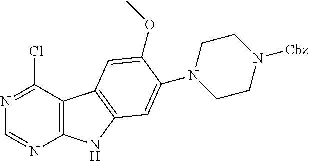 [SMILES: COc1cc2c(cc1N1CCN(C(=O)OCc3ccccc3)CC1)[nH]c1ncnc(Cl)c12]

To a stirred solution of benzyl 4-(6-methoxy-4-oxo-4,9-dihydro-3H-pyrimido[4,5-b]indol-7-yl)piperazine-1-carboxylate (28.00 g, 64.595 mmol) in dioxane (560.00 mL) was added POCl₃ (78.27 mL, 839.735 mmol) at room temperature. The resulting mixture was stirred for 6 h at 90° C. under nitrogen atmosphere. The mixture was allowed to cool down to room temperature and concentrated under reduced pressure. The residue was basified to pH 7 with saturated NaHCO₃ (aq.). The resulting mixture was extracted with EtOAc (2×1 L) and dried over anhydrous Na₂SO₄. After filtration, the filtrate was concentrated under reduced pressure. The precipitated solid was collected by filtration and washed with diethyl ether (3×100 mL) to give the product (30 g, 96%). [M+H]⁺=452.3.

### Step 13: benzyl (R)-4-(4-(4-(1-(3-(tert-butyl)-1,2,4-oxadiazole-5-carboxamido)ethyl)-3-methylphenyl)-6-methoxy-9H-pyrimido[4,5-b]indol-7-yl)piperazine-1-carboxylate

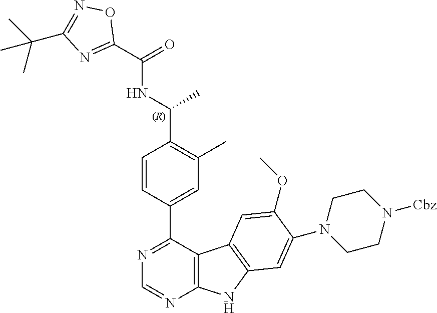

To a stirred solution of benzyl 4-(4-chloro-6-methoxy-9H-pyrimido[4,5-b]indol-7-yl)piperazine-1-carboxylate (1.10 g, 2.439 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (the compound was obtained through the same way in WO2021219070A) (1.21 g, 2.927 mmol) in DMF/H₂O (5/1, 20 mL) were added K₃PO₄ (1.04 g, 4.879 mmol) and Pd(dppf)Cl₂ (0.18 g, 0.244 mmol) at room temperature under nitrogen atmosphere. The resulting mixture was stirred overnight at 90° C. under nitrogen atmosphere. The mixture was allowed to cool down to room temperature and diluted with water (250 mL). The resulting mixture was extracted with EtOAc (3×300 mL). The combined organic layers were washed with water (1×300 mL) and dried over anhydrous Na₂SO₄. After filtration, the filtrate was concentrated under reduced pressure. The residue was purified by silica gel column chromatography, eluted with CH₂Cl₂/MeOH (20:1) to afford the product (850 mg, 50%). [M+H]⁺=703.4.

### Step 14: (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide

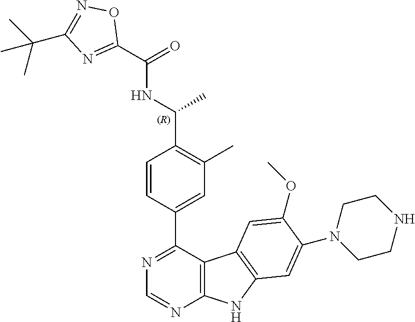

A solution of benzyl (R)-4-(4-(4-(1-(3-(tert-butyl)-1,2,4-oxadiazole-5-carboxamido)ethyl)-3-methylphenyl)-6-methoxy-9H-pyrimido[4,5-b]indol-7-yl)piperazine-1-carboxylate (700.00 mg, 0.996 mmol) in TFA (65.00 mL) was stirred overnight at 60° C. under nitrogen atmosphere. The resulting mixture was concentrated under reduced pressure. The residue was purified by reverse phase flash chromatography with the following conditions: column, C18; mobile phase, 0.1% acetic acid in water, 10% to 50% gradient in 30 min; detector, UV 254 nm. This afforded the product (530 mg, 87%). [M+H]⁺=569.1.

### Step 15: 3-(tert-butyl)-N—((R)-1-(4-(7-(4-((1S,3s)-3-(4-(((R)-2,6-dioxopiperidin-3-yl)oxy)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide and 3-(tert-butyl)-N—((R)-1-(4-(7-(4-((1R,3s)-3-(4-(((S)-2,6-dioxopiperidin-3-yl)oxy)phenyl)cyclobutyl)piperazin-1-yl)-6-methoxy-9H-pyrimido[4,5-b]indol-4-yl)-2-methylphenyl)ethyl)-1,2,4-oxadiazole-5-carboxamide

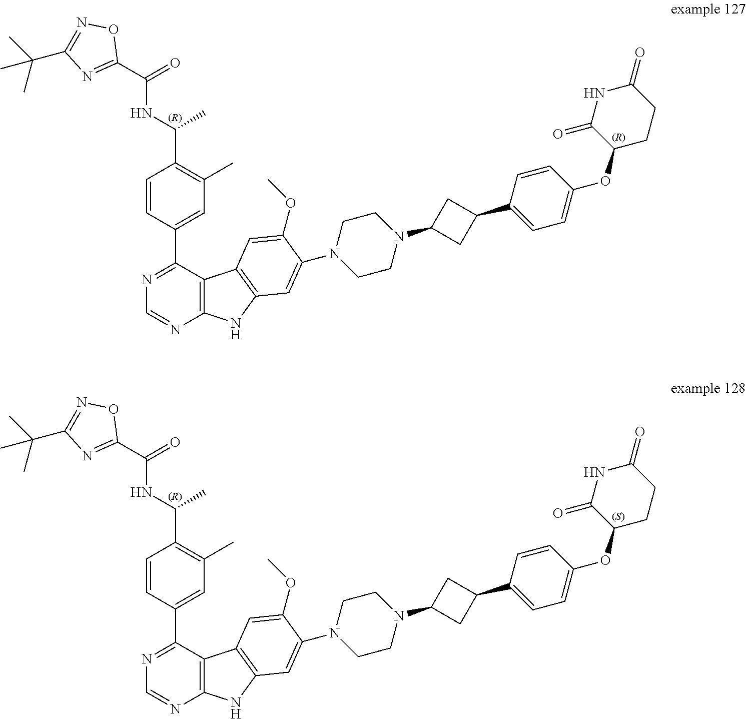

To a stirred solution of (R)-3-(tert-butyl)-N-(1-(4-(6-methoxy-7-(piperazin-1-yl)-9H-pyrimido[4,5-b]indol-4-yl)-2-methylphenyl)ethyl)-1,2,4-oxadiazole-5-carboxamide (150 mg, 0.264 mmol) and 3-(4-(3-oxocyclobutyl)phenoxy)piperidine-2,6-dione (100 mg, 0.366 mmol) in THF/DMF(2:1, 15 mL), titanium tetraisopropanolate (1.0 mL) was added in portions and the resulting mixture was stirred at room temperature for 16 h until LC-MS indicated all the starting material was consumed. The mixture was concentrated and purified with SiO₂-gel column (DCM:MeOH=10:1). Then the residue was purified by reverse phase flash chromatography with the following conditions: column, C18; mobile phase A, 0.1% FA in water, mobile phase B, ACN, 10% to 50% gradient in 25 min; detector, UV 214 nm. This gave example 127 (7 mg, 3%) and example 128 (1.2 mg, 0.6%). Example 127: ¹H NMR (500 MHz, DMSO) δ 12.08 (s, 1H), 10.84 (s, 1H), 9.87 (d, J=7.7 Hz, 1H), 8.78 (d, J=5.2 Hz, 1H), 8.26 (s, 1H), 7.75-7.68 (m, 2H), 7.65 (d, J=7.9 Hz, 1H), 7.20 (s, 1H), 7.09 (d, J=8.7 Hz, 2H), 6.95 (s, 1H), 6.88 (d, J=8.7 Hz, 2H), 5.35-5.31 (m, 1H), 5.09 (dd, J=10.8, 5.0 Hz, 1H), 3.59 (s, 3H), 3.08-2.96 (m, 6H), 2.72-2.60 (m, 2H), 2.58-2.53 (m, 1H), 2.51 (t, J=4.6 Hz, 1H), 2.49-2.45 (m, 3H), 2.39-2.34 (m, 4H), 2.17-1.99 (m, 2H), 1.77 (q, J=9.0 Hz, 2H), 1.51 (d, J=7.0 Hz, 3H), 1.30 (s, 9H); [M+H]⁺=826.6.

Example 128: ¹H NMR (500 MHz, DMSO) δ 12.08 (s, 1H), 10.85 (s, 1H), 9.87 (d, J=7.5 Hz, 1H), 8.78 (s, 1H), 7.74-7.69 (m, 2H), 7.65 (d, J=8.0 Hz, 1H), 7.21 (s, 1H), 7.16 (d, J=8.6 Hz, 2H), 6.96 (s, 1H), 6.90 (d, J=8.5 Hz, 2H), 5.36-5.30 (m, 1H), 5.09 (dd, J=10.8, 5.1 Hz, 1H), 3.58 (s, 3H), 3.13-2.95 (m, 4H), 2.93-2.82 (m, 2H), 2.70-2.60 (m, 2H), 2.59-2.54 (m, 2H), 2.53-2.45 (m, 3H), 2.34-2.26 (m, 4H), 2.18-1.98 (m, 4H), 1.51 (d, J=6.8 Hz, 3H), 1.30 (s, 9H); [M+H]⁺=826.6.

### Example 132: 3-(tert-butyl)-N-((1R)-1-(4-(7-(4-((1-(4-(2,4-dioxotetrahydropyrimidin-1(2H)-yl)phenyl)piperidin-4-yl)methyl)piperazin-1-yl)-6,7,8,9-tetrahydro-5H-pyrimido[4,5-b]indol-4-yl)-2-methylphenyl)ethyl)-1,2,4-oxadiazole-5-carboxamide

### Step 1: 6-(2-(1,4-dioxaspiro[4.5]decan-8-ylidene)hydrazineyl)pyrimidin-4(3H)-one

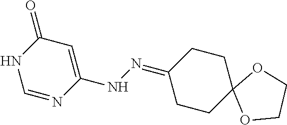 [SMILES: O=c1cc(NN=C2CCC3(CC2)OCCO3)nc[nH]1]

A mixture of 6-hydrazinyl-3H-pyrimidin-4-one (25 g, 198.23 mmol) and 1,4-dioxaspiro[4.5]decan-8-one (37.2 g, 237.87 mmol) in EtOH (250 mL) was refluxed for 2.5 h. The mixture was allowed to cool down to room temperature. The precipitated solid was collected by filtration and washed with diethyl ether (2×200 mL). This resulted in the product (42.8 g, 81%). [M+H]⁺=265.2.

### Step 2: 5,6,8,9-tetrahydrospiro[pyrimido[4,5-b]indole-7,2′-[1,3]dioxolan]-4(3H)-one

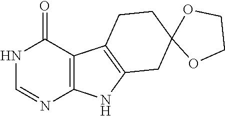 [SMILES: O=c1[nH]cnc2[nH]c3c(c12)CCC1(C3)OCCO1]

A mixture of 6-(2-(1,4-dioxaspiro[4.5]decan-8-ylidene)hydrazineyl)pyrimidin-4(3H)-one (8 g, 30.27 mmol) in phenoxybenzene (160 mL) was stirred for 4 h at 250° C. under nitrogen atmosphere. The mixture was allowed to cool down to room temperature. The resulting mixture was diluted with hexane (550 mL). The precipitated solid was collected by filtration and washed with hexane (3×100 mL) to afford the product (7.4 g, 99%). [M+H]⁺=248.1.

### Step 3: 4-chloro-5,6,8,9-tetrahydrospiro[pyrimido[4,5-b]indole-7,2′-[1,3]dioxolane]

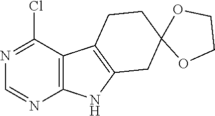 [SMILES: Clc1ncnc2[nH]c3c(c12)CCC1(C3)OCCO1]

A mixture of 5,6,8,9-tetrahydrospiro[pyrimido[4,5-b]indole-7,2′-[1,3]dioxolan]-4(3H)-one (4.4 g, 17.80 mmol) in POCl₃ (66 mL) was stirred for 1 h at 100° C. The resulting mixture was concentrated under reduced pressure. The residue was diluted with ice water (200 mL). The mixture was basified to pH=8 with saturated Na₂CO₃ (aq.). The resulting mixture was extracted with EtOAc (3×300 mL). The combined organic layers were washed with brine (2×200 mL) and dried over anhydrous Na₂SO₄. After filtration, the filtrate was concentrated under reduced pressure. The residue was purified by silica gel column chromatography, eluted with CH₂Cl₂/MeOH (80:1 to 40:1) to afford the product (4.6 g, 98%). [M+H]⁺=265.9.

### Step 4: 4-chloro-5,6,8,9-tetrahydro-7H-pyrimido[4,5-b]indol-7-one

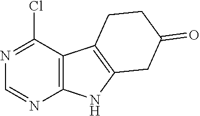 [SMILES: O=C1CCc2c([nH]c3ncnc(Cl)c23)C1]

A mixture of 4-chloro-5,6,8,9-tetrahydrospiro[pyrimido[4,5-b]indole-7,2′-[1,3]dioxolane] (3.7 g, 13.925 mmol) in 2 M HCl (aq.) (40 mL) and THF (40 mL) was stirred for 16 h at room temperature. The resulting mixture was concentrated under reduced pressure and washed with diethyl ether (300 mL) to afford the product (3.0 g, 97%). [M+H]⁺=221.9.

### Step 5: (R)-3-(tert-butyl)-N-(1-(2-methyl-4-(7-oxo-6,7,8,9-tetrahydro-5H-pyrimido[4,5-b]indol-4-yl)phenyl)ethyl)isoxazole-5-carboxamide

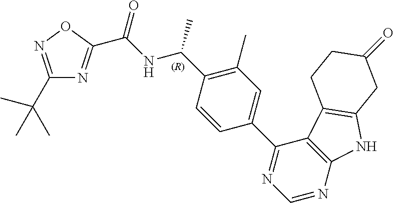

To a solution of 4-chloro-5,6,8,9-tetrahydro-7H-pyrimido[4,5-b]indol-7-one (4 g, 18.05 mmol) and 3-tert-butyl-N-[(1R)-1-[2-methyl-4-(4,4,5,5-tetramethyl-1,3,2-dioxaborolan-2-yl)phenyl]ethyl]-1,2,4-oxadiazole-5-carboxamide (8.95 g, 21.66 mmol) in DMF (80 mL) and H₂O (16 mL) were added K₃PO₄ (7.66 g, 36.09 mmol) and Pd(dppf)Cl₂ (1.32 g, 1.81 mmol). After stirring for 16 h at 80° C. under nitrogen atmosphere, the resulting mixture was cooled down to ambient temperature and diluted with water (600 mL). The resulting mixture was extracted with EtOAc (4×300 mL). The combined organic layers were washed with brine (2×400 mL) and dried over anhydrous Na₂SO₄. After filtration, the filtrate was concentrated under reduced pressure. The residue was purified by silica gel column chromatography, eluted with CH₂Cl₂/EtOAc (1:1 to 1:4) to afford the product (2.3 g, 27%). [M+H]⁺=473.4.

### Step 6: tert-butyl 4-(4-(4-((R)-1-(3-(tert-butyl)-1,2,4-oxadiazole-5-carboxamido)ethyl)-3-methylphenyl)-6,7,8,9-tetrahydro-5H-pyrimido[4,5-b]indol-7-yl)piperazine-1-carboxylate

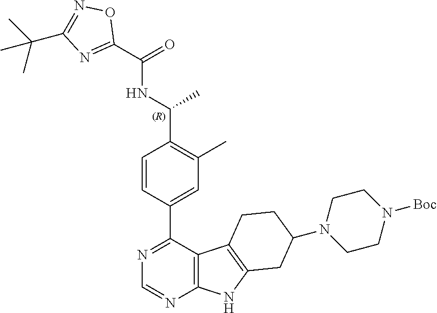

To a stirred mixture of (R)-3-(tert-butyl)-N-(1-(2-methyl-4-(7-oxo-6,7,8,9-tetrahydro-5H-pyrimido[4,5-b]indol-4-yl)phenyl)ethyl)isoxazole-5-carboxamide (2.2 g, 4.66 mmol) and tert-butyl piperazine-1-carboxylate (950 mg, 5.122 mmol) in MeOH (25 mL) was added NaBH₃CN (1.5 g, 23.280 mmol) in portions at room temperature. The resulting mixture was stirred for 16 h at 50° C. and concentrated under reduced pressure. The residue was purified by silica gel column chromatography, eluted with CH₂Cl₂/MeOH (40:1 to 10:1) to afford the product (1.7 g, 57%). [M+H]⁺=643.3.

### Step 7: 3-(tert-butyl)-N-((1R)-1-(2-methyl-4-(7-(piperazin-1-yl)-6,7,8,9-tetrahydro-5H-pyrimido[4,5-b]indol-4-yl)phenyl)ethyl)-1,2,4-oxadiazole-5-carboxamide trifluoroacetate

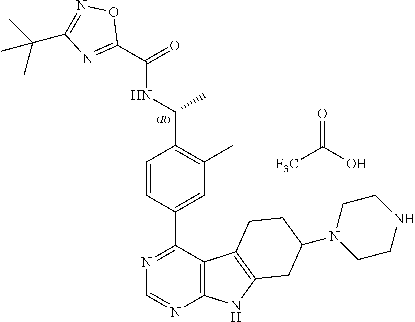

A mixture of tert-butyl 4-(4-(4-((R)-1-(3-(tert-butyl)-1,2,4-oxadiazole-5-carboxamido)ethyl)-3-methylphenyl)-6,7,8,9-tetrahydro-5H-pyrimido[4,5-b]indol-7-yl)piperazine-1-carboxylate (1.7 g, 2.645 mmol) in 4 M HCl in 1,4-dioxane (17 mL) and DCM (17 mL) was stirred for 1 h at room temperature. The resulting mixture was concentrated under reduced pressure. The residue was purified by reverse phase flash chromatography with the following conditions: Column: Spherical C18, 20-40 um, 330 g; Mobile Phase A: Water (plus 0.05% TFA); Mobile Phase B: ACN; Flow rate: 80 mL/min; Gradient: 5% B, 10 min, 15% B-40% B gradient in 30 min; Detector: 220 nm. The fractions containing the desired product were collected at 34% B and concentrated under reduced pressure to afford the product (656 mg, 38%). ¹H NMR (400 MHz, DMSO) δ 12.90 (s, 1H), 9.96 (t, J=7.7 Hz, 1H), 9.23 (s, 1H), 8.94 (d, J=1.7 Hz, 1H), 7.72-7.66 (m, 1H), 7.66-7.60 (m, 1H), 7.58 (d, J=1.9 Hz, 1H), 6.45-6.37 (m, 10H), 5.36 (td, J=7.2, 4.1 Hz, 1H), 3.18 (s, 1H), 3.06 (d, J=17.4 Hz, 1H), 2.95 (d, J=10.3 Hz, 1H), 2.80 (dd, J=23.3, 10.8 Hz, 1H), 2.54-2.50 (m, 3H), 2.34 (s, 1H), 1.57 (d, J=7.0 Hz, 3H), 1.37 (d, J=2.2 Hz, 9H); [M+H]⁺=543.4.

### Step 8: 3-(tert-butyl)-N-((1R)-1-(4-(7-(4-((1-(4-(2,4-dioxotetrahydropyrimidin-1(2H)-yl)phenyl)piperidin-4-yl)methyl)piperazin-1-yl)-6,7,8,9-tetrahydro-5H-pyrimido[4,5-b]indol-4-yl)-2-methylphenyl)ethyl)-1,2,4-oxadiazole-5-carboxamide

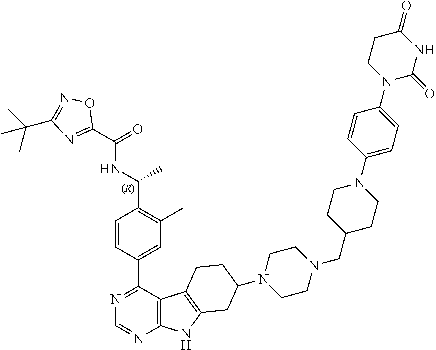

To a stirred solution of 3-(tert-butyl)-N-((1R)-1-(2-methyl-4-(7-(piperazin-1-yl)-6,7,8,9-tetrahydro-5H-pyrimido[4,5-b]indol-4-yl)phenyl)ethyl)-1,2,4-oxadiazole-5-carboxamide trifluoroacetate (80 mg, 0.122 mmol) and 1-(4-(2,4-dioxotetrahydropyrimidin-1(2H)-yl)phenyl)piperidine-4-carbaldehyde (55 mg, 0.183 mmol) in THF/DMF (2:1, 6 mL), titanium tetraisopropanolate (0.5 mL) was added in portions and the resulting mixture was stirred at room temperature for 16 h until LC-MS indicated all the starting material was consumed. The mixture was concentrated and purified with SiO₂-gel column (DCM:MeOH=10:1). Then the residue was purified by reverse phase flash chromatography with the following conditions: column, C18; mobile phase A, 0.1% FA in water, mobile phase B, ACN, 10% to 30% gradient in 25 min; detector, UV 214 nm. This gave the product (33 mg, 33%). ¹H NMR (500 MHz, DMSO) δ 12.33 (s, 1H), 10.27 (s, 1H), 9.95 (t, J=7.7 Hz, 1H), 8.78 (s, 1H), 7.64 (d, J=8.0 Hz, 1H), 7.60 (d, J=7.8 Hz, 1H), 7.55 (d, J=8.1 Hz, 1H), 7.17 (d, J=8.8 Hz, 2H), 6.98 (d, J=6.3 Hz, 2H), 5.36 (td, J=7.2, 2.2 Hz, 1H), 3.80-3.66 (m, 6H), 3.05-2.93 (m, 3H), 2.92-2.81 (m, 2H), 2.80-2.61 (m, 8H), 2.60-2.50 (m, 7H), 2.35-2.23 (m, 1H), 1.95-1.74 (m, 4H), 1.55 (d, J=6.6 Hz, 3H), 1.36 (d, J=7.5 Hz, 9H), 1.34-1.21 (m, 2H); [M+H]⁺=828.6.

### Example 133: 3-(tert-butyl)-N-((1R)-1-(4-(7-(4-(3-(4-((R)-2,6-dioxopiperidin-3-yl)phenyl)cyclobutyl)piperazin-1-yl)-6,7,8,9-tetrahydro-5H-pyrimido[4,5-b]indol-4-yl)-2-methylphenyl)ethyl)-1,2,4-oxadiazole-5-carboxamide

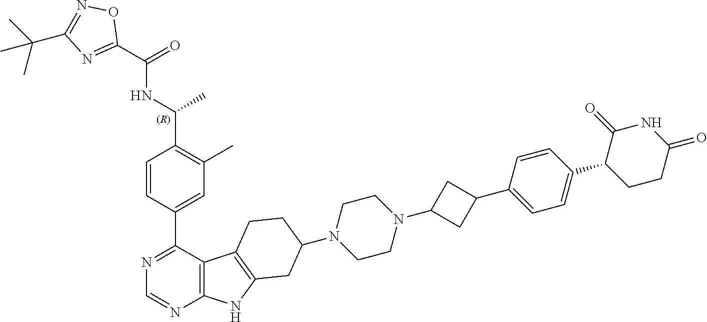

The titled compound was synthesized in the procedures similar to Example 132. ¹H NMR (500 MHz, DMSO) δ 12.21 (s, 1H), 10.82 (s, 1H), 9.93 (dd, J=16.4, 7.6 Hz, 1H), 8.75 (s, 1H), 7.62 (t, J=6.4 Hz, 2H), 7.54 (d, J=7.0 Hz, 1H), 7.25 (d, J=7.3 Hz, 2H), 7.19 (d, J=7.7 Hz, 2H), 5.43-5.29 (m, 1H), 3.83 (dd, J=11.6, 4.9 Hz, 2H), 3.35-3.15 (m, 5H), 3.02-2.89 (m, 3H), 2.87-2.79 (m, 3H), 2.70-2.57 (m, 5H), 2.56-2.53 (m, 2H), 2.45-2.36 (m, 2H), 2.28-2.11 (m, 4H), 2.06-1.96 (m, 2H), 1.83-1.73 (m, 1H), 1.60-1.50 (m, 3H), 1.36 (d, J=8.0 Hz, 9H); [M+H]⁺=784.5.

### Cell Degradation

### Cell Treatment

TMD-8 cells were seeded at 20000 cells/well at a volume of 15 μl/well in cell culture medium [RPMI1640 (Gibco, phenol red free, Cat #11835-030), 10% heat-inactive FBS, 1% PS (Gibco, Cat #10378)] in Corning 96 well plate (Cat #3799). TMD-8 cells were treated with compounds diluted in 0.2% DMSO, dilution was done according to the following protocol: (1) making 500× stock solution in DMSO from 1 mM by 6-fold dilution, total 8 doses were included; (2) making 2× solution in cell culture medium by transferring 0.5 μl 500× stock solution into 125 μl medium; (3) adding 15 μl of 2× solution to cells for incubation of 6 h.

### HTFR Assay

After 6 h treatment, 10 μl 4× lysis buffer was added to each well; the plate was sealed and incubated for 30 min at room temperature on a plate shaker; Once the cells was lysed, 16 μL of cell lysate were transferred to a PE 384-well HTRF detection plate; 4 μL of pre-mixed HTRF antibodies were added to each well; the plate was covered with a plate sealer, and then spinned at 1000 rpm for 1 min, then incubated overnight at room temperature; the results were read on BMG PheraStar with HTRF protocol (337 nm-665 nm-620 nm).

The inhibition (degradation) percentage of the compound was calculated by the following equation: Inhibition percentage of Compound=100−100× (Signal-low control)/(High control-low control), wherein signal=each test compound group
Low control=only lysis buffer without cells, indicating that BTK is completely degraded;
High control=Cell group with added DMSO and without compound, indicating microplate readings without BTK degradation;
Dmax is the maximum percentage of inhibition (degradation).

The IC₅₀ (DC₅₀) value of a compound can be obtained by fitting the following equation

$$Y = Bottom + ( TOP - Bottom ) / ( 1 + ( ( I {C}_{50} / X ) \hat{} hillslope ) )$$

wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1 *; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

### HEK-293 Cell Treatment

HEK-293 cells were seeded at 2000 cells/well at a volume of 50 ul/well in cell culture medium [DMEM (Gibco, Cat #11965-092), 10% heat-inactive FBS (Gibco, Cat #10099), 1% PS (Gibco, Cat #10378)] in Corning 96 well plate (Cat #3903), and then incubated overnight. HEK-293 cells were treated with compounds diluted in 0.2% DMSO, dilution was done according to the following protocol: (1) making 500× stock solution in DMSO from 5 mM by 4-fold dilution, total 8 doses were included; (2) making 2× solution in cell culture medium by transferring 0.5 ul 500× stock solution into 125 μl medium; (3) adding 50 μl of 2× solution to cells for incubation of 72 h.

### Cytotoxicity Detection

25 μl of the CellTiter-Glo®Reagent [(Promega) -Cat No. G7572] was added to each well in the 96-well plate. The contents were mixed for 2 minutes on an orbital shaker to induce cell lysis. The plate was then allowed to incubate at room temperature for 10 minutes to stabilize luminescent signal. Luminescence was recorded on BMG PheraStar with luminescence protocol.

### IC50 Calculation

The inhibition percentage of the compound was calculated by the following equation: Inhibition percentage of Compound=100−100×(Signal-low control)/(High control-low control), wherein signal=each test compound group
Low control=only medium group (without cells), indicating that cells proliferation is completely inhibited;
High control=Cell group with added DMSO and without compound, indicating cells proliferation with no inhibition;
Imax is the maximum percentage of inhibition.

The IC50 value of a compound can be obtained by fitting the following equation

$$Y = Bottom + ( TOP - Bottom ) / ( 1 + ( ( IC 50 / X ) \hat{} hillslope ) )$$

Wherein, X and Y are known values, and IC50, Hillslope, Top and Bottom are the parameters obtained by fitting with software. Y is the inhibition percentage (calculated from the equation), X is the concentration of the compound; IC50 is the concentration of the compound when the 50% inhibition is reached. The smaller the IC50 value is, the stronger the inhibitory ability of the compound is. Vice versa, the higher the IC50 value is, the weaker the ability the inhibitory ability of the compound is; Hillslope represents the slope of the fitted curve, generally around 1 *; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation and HEK293 result for Example 1 to Example 133

| Example | HEK293 (nM) | DC50 (nM) |
|---|---|---|
| 1 | >10000.0 | 4.94 |
| 2 | 3953.8 | >2000 |
| 3 | >10000.0 | 2.15 |
| 4 | 3655 | 0.294 |
| 5 | 3252.3 | 0.731 |
| 6 | >10000.0 | 0.394 |
| 7 | >10000.0 | 1.27 |
| 8 | >10000.0 | 0.481 |
| 9 | >10000.0 | 0.36 |
| 10 | >10000.0 | 1.51 |
| 11 | >10000.0 | 9.78 |
| 12 | 3638.4 | 0.613 |
| 13 | 3264.9 | 1.05 |
| 14 | 3336.7 | 0.495 |
| 15 | >10000.0 | 0.616 |
| 16 | >10000.0 | 0.347 |
| 17 | >10000.0 | 0.341 |
| 18 | >10000.0 | 0.607 |
| 19 | 5649.1 | 0.564 |
| 20 | 3521.3 | 1.3 |
| 21 | 3595.1 | 0.609 |
| 22 | >10000.0 | 0.211 |
| 23 | 2895.9 | 0.608 |
| 24 | >10000.0 | 0.47 |
| 25 | >10000.0 | 0.872 |
| 26 | >10000.0 | 2.2 |
| 27 | >10000.0 | 0.299 |
| 28 | >10000.0 | 0.309 |
| 29 | >10000.0 | 3.16 |
| 30 | >10000.0 | 4.55 |
| 31 | >10000.0 | 1.43 |
| 32 | >10000.0 | 9.03 |
| 33 | >10000.0 | 3.67 |
| 34 | 1440.5 | 1.23 |
| 35 | >10000.0 | 0.15 |
| 36 | 3411.9 | 0.804 |
| 37 | >10000.0 | 57.65 |
| 38 | 3463.8 | 0.395 |
| 39 | 4021.4 | 0.725 |
| 40 | >10000.0 | 2.12 |
| 41 | 3350.5 | 1.85 |
| 42 | 6726.8 | 1.29 |
| 43 | >10000.0 | 1.22 |
| 44 | >10000.0 | 1.38 |
| 45 | >10000.0 | 1.05 |
| 46 | >10000.0 | 0.366 |
| 47 | >10000.0 | 1.7 |
| 48 | >10000.0 | 0.874 |
| 49 | >10000.0 | 1.08 |
| 50 | >10000.0 | 0.171 |
| 51 | >10000.0 | 0.987 |
| 52 | >10000.0 | 0.361 |
| 53 | >10000.0 | 2.26 |
| 54 | >10000.0 | 3.05 |
| 55 | >10000.0 | 6.97 |
| 56 | >10000.0 | 10.27 |
| 57 | >10000.0 | 9.6 |
| 58 | >10000.0 | 1.87 |
| 59 | >10000.0 | 4.55 |
| 60 | >10000.0 | 4.97 |
| 61 | >10000.0 | 5.52 |
| 62 | >10000.0 | 0.414 |
| 63 | >10000.0 | 6.82 |
| 64 | >10000.0 | 3.02 |
| 65 | >10000.0 | 4.24 |
| 66 | >10000.0 | 4.1 |
| 67 | >10000.0 | 1.27 |
| 68 | >10000.0 | 1.6 |
| 69 | >10000.0 | 0.266 |
| 70 | 1990.9 | 140.5 |
| 71 | >10000.0 | 9.24 |
| 72 | >10000.0 | 15.94 |
| 73 | >10000.0 | 1.27 |
| 74 | >10000.0 | 2.04 |
| 75 | >10000.0 | >2000 |
| 76 | >10000.0 | 1.6 |
| 77 | >10000.0 | 8.48 |
| 78 | >10000.0 | >2000 |
| 79 | >10000.0 | 15.9 |
| 80 | >10000.0 | 16.61 |
| 81 | >10000.0 | >2000 |
| 82 | >10000.0 | 3.17 |
| 83 | >10000.0 | 2.42 |
| 84 | >10000.0 | 8.15 |
| 85 | >10000.0 | >2000 |
| 86 | >10000.0 | >2000 |
| 87 | >10000.0 | >2000 |
| 88 | >10000.0 | >2000 |
| 89 | >10000.0 | 0.55 |
| 90 | >10000.0 | 0.86 |
| 91 | >10000.0 | >2000 |
| 92 | >10000.0 | 3.08 |
| 93 | >10000.0 | 2.51 |
| 94 | 419.8 | 0.768 |
| 95 | >10000.0 | 0.49 |
| 96 | >10000.0 | 2.98 |
| 97 | >10000.0 | 0.407 |
| 98 | 7285 | 1.97 |
| 99 | >10000.0 | 3.25 |
| 100 | 3424.8 | 1.02 |
| 101 | 956.3 | 1.52 |
| 102 | 1024.1 | 4.88 |
| 103 | 1063.8 | 3.05 |
| 104 | >10000.0 | 1.16 |
| 105 | >10000.0 | 0.512 |
| 106 | >10000.0 | 0.324 |
| 107 | 1025.5 | 2.99 |
| 108 | 948.9 | 2.64 |
| 109 | 1013.2 | 6.07 |
| 110 | 2002.5 | 1.25 |
| 111 | 1365.2 | 2.89 |
| 112 | 1893.9 | 2.47 |
| 113 | 1117.3 | 3.46 |
| 114 | 922.9 | 1.89 |
| 115 | >10000.0 | 0.759 |
| 116 | >10000.0 | 0.509 |
| 117 | >10000.0 | 0.981 |
| 118 | >10000.0 | 1.73 |
| 119 | >10000.0 | 2.51 |
| 120 | >10000.0 | 1.25 |
| 121 | >10000.0 | 1.58 |
| 122 | >10000.0 | 0.395 |
| 123 | >10000.0 | 0.192 |
| 124 | >10000.0 | 0.838 |
| 125 | >10000.0 | 0.197 |
| 126 | >10000.0 | 0.449 |
| 127 | >10000.0 | 2.49 |
| 128 | >10000.0 | 2.12 |
| 129 | 2042 | >2000 |
| 130 | >10000.0 | 63.25 |
| 131 | >10000.0 | 27.47 |
| 132 | >10000.0 | >2000 |
| 133 | >10000.0 | >2000 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

1. A compound of Formula (I): 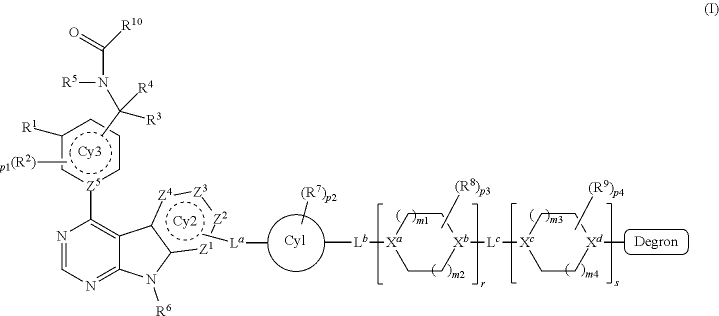
or a pharmaceutically acceptable salt thereof, or a stereoisomer thereof,
wherein:
Cy1 is a 4-, 5-, 6-, 7-, 8-, 9-, 10-, 11- or 12-membered ring comprising 0-3 heteroatoms selected from nitrogen, oxygen and sulfur;
Cy2 is a ring fused to the structure 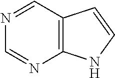
in Formula (I), said ring is an aromatic ring or a non-aromatic ring (preferably aromatic ring);
Cy3 is a saturated ring or unsaturated ring (preferably aromatic ring);
L^{a}, L^{b} and L^{c} are each independently a single bond, —O—, —S—, —C(O)—, —(CR^{c}R^{d})ₙ— or —NR^{c}—;
the [symbol] moiety is an E3 Ubiquitin ligase moiety selected from 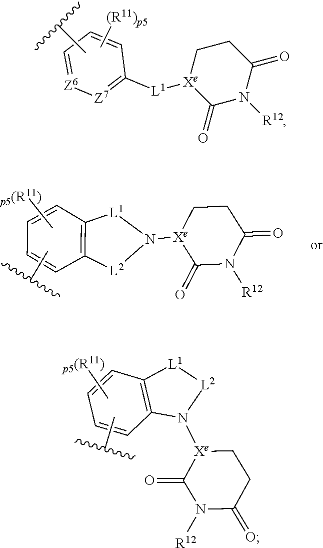
L¹ and L² are each independently a single bond, —O—, —S—, —C(O)—, —(CR^{c}R^{d})ₙ— or —NR^{c}—;
Z¹ is N or CR^{za}a if Z¹ is a part of a double bond in the ring; or Z¹ is NR^{za} or C(R^{za})₂ if Z¹ is not a part of a double bond in the ring;
Z² is N or CR^{za} if Z² is a part of a double bond in the ring; or Z² is NR^{za} or C(R^{za})₂ if Z² is not a part of a double bond in the ring;
Z³ is N or CR^{za} if Z³ is a part of a double bond in the ring; or Z³ is NR^{za} or C(R^{za})₂ if Z³ is not a part of a double bond in the ring;
Z⁴ is N or CR^{za} if Z⁴ is a part of a double bond in the ring; or Z⁴ is NR^{za} or C(R^{za})₂ if Z⁴ is not a part of a double bond in the ring; provided that not all Z¹, Z², Z³ and Z⁴ are ═N—;
Z⁵ is C if Z⁵ is a part of a double bond in the ring; or Z⁵ is N or CR^{za} if Z⁵ is not a part of a double bond in the ring;
Z⁶ and Z⁷ are each independently CR^{za} or N;
X^{a}, X^{b}, X^{c}, X^{d} and X^{e} are each independently CR¹³ or N;
each of occurrence, R¹, R², R³, R⁴, R⁷, R⁸, R⁹, R¹¹, R¹³ and R^{za} are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈ alkyl, —C₁₋₈alkyoxy, cycloalkyl, heterocyclyl, aryl, or heteroaryl;
or R¹ and R³, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈ alkyl, —C₁₋₈alkyoxy, cycloalkyl, heterocyclyl, aryl, or heteroaryl;
R⁵, R⁶ and R¹² are each independently hydrogen, —C₁₋₈alkyl, —C₂₋₈alkenyl, —C₂₋₈alkynyl, cycloalkyl, heterocyclyl, aryl or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R¹⁰ is 5- or 6-membered aromatic ring comprising 0-3 heteroatoms selected from nitrogen, oxygen and sulfur; said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈ alkenyl, —C₂₋₈alkynyl, cycloalkyl, heterocyclyl, aryl, or heteroaryl is optionally substituted with —C₁₋₈alkyl, halogen, hydroxy, -haloC₁₋₈alkyl, —C₁₋₈alkyoxy, cycloalkyl, heterocyclyl, aryl, or heteroaryl;
or R⁵ and R¹⁰, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈ alkenyl, —C₂₋₈alkynyl, cycloalkyl, heterocyclyl, aryl, heteroaryl, oxo, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈ alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
m1, m2, m3, m4, p1, p2, p3, p4 and p5 are each independently 0, 1, 2, 3 or 4;
n is 0, 1, 2, 3, 4, 5 or 6;
r is 0 or 1;
s is 0 or 1;
R^{a} and R^{b} are each independently hydrogen, —C₁₋₈alkyl, —C₂₋₈alkenyl, —C₂₋₈alkynyl, cycloalkyl, heterocyclyl, aryl, or heteroaryl; or
R^{a} and R^{b} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂;
R^{c} and R^{d} are each independently hydrogen, —C₁₋₈alkyl, —C₂₋₈alkenyl, —C₂₋₈alkynyl, cycloalkyl, heterocyclyl, aryl, or heteroaryl; or
R^{c} and R^{d} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

2. The compound according to claim 1, wherein the compound is selected from formula (II) and formula (III), 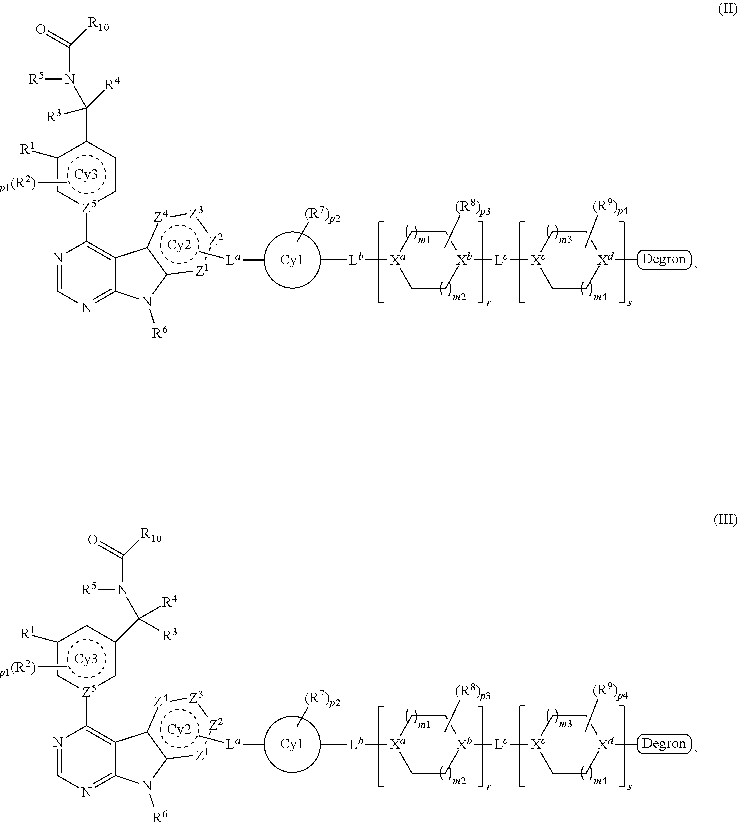
Cy1, Cy2, Cy3, L^{a}, L^{b}, L^{c}, [symbol] , Z¹, Z², Z³, Z⁴, Z⁵, X^{a}, X^{b}, X^{c}, X^{d}, R¹, R², R³, R⁴, R⁵, R⁶, R⁷, R⁸, R⁹, R¹⁰, m1, m2, m3, m4, p1, p2, p3, p4, r and s are defined as claim 1.

3. The compound according to claim 1, wherein the compound is selected from formula (IV) and formula (V), 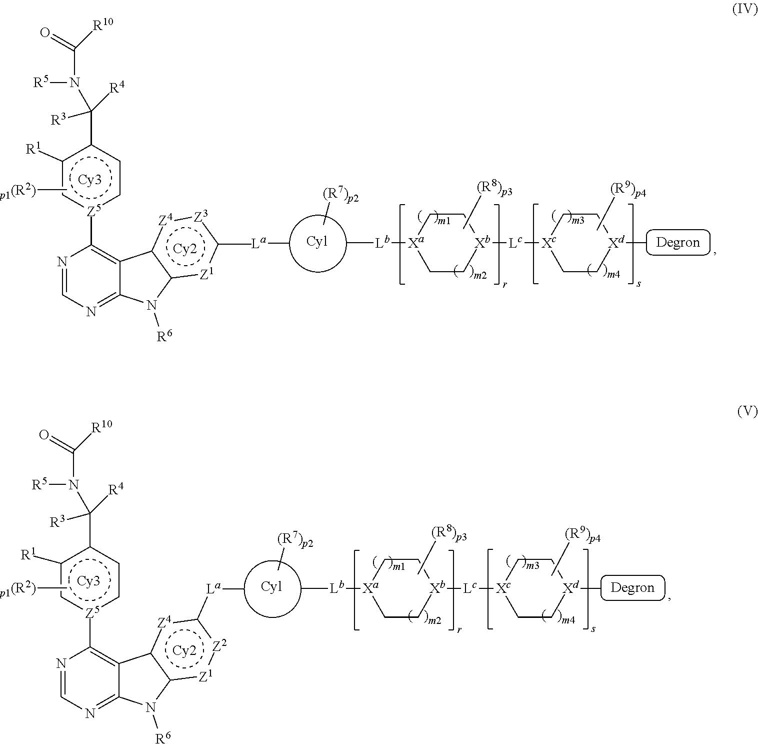
Cy1, Cy2, Cy3, L^{a}, L^{b}, L^{c}, [symbol] , Z¹, Z², Z³, Z⁴, Z⁵, X^{a}, X^{b}, X^{c}, X^{d}, R¹, R², R³, R⁴, R⁵, R⁶, R⁷, R⁸, R⁹, R¹⁰, m1, m2, m3, m4, p1, p2, p3, p4, r and s are defined as claim 1.

4. The compound according to claim 1, wherein Cy1 is 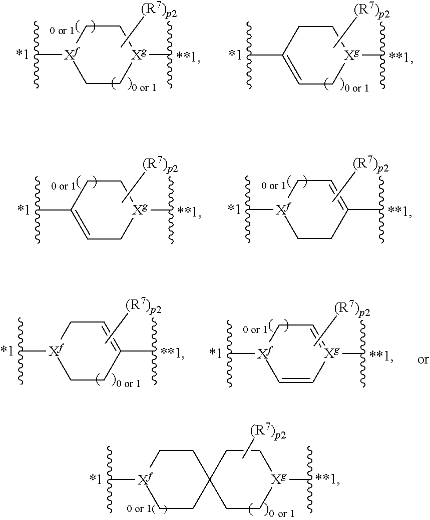
wherein X^{f} and X^{g} are each independently CR¹¹ or N;
*1 refers to the position attached to the 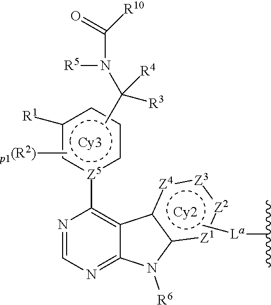
moiety, and **1 refers to the position attached to 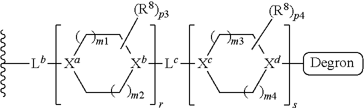
moiety.

5. The compound according to claim 1, wherein Cy1 is

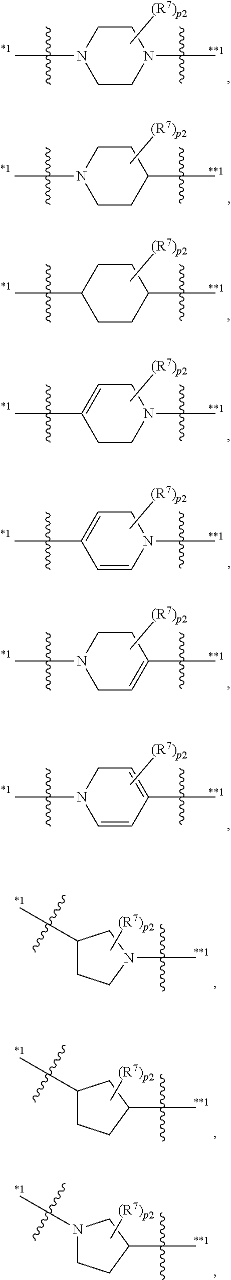

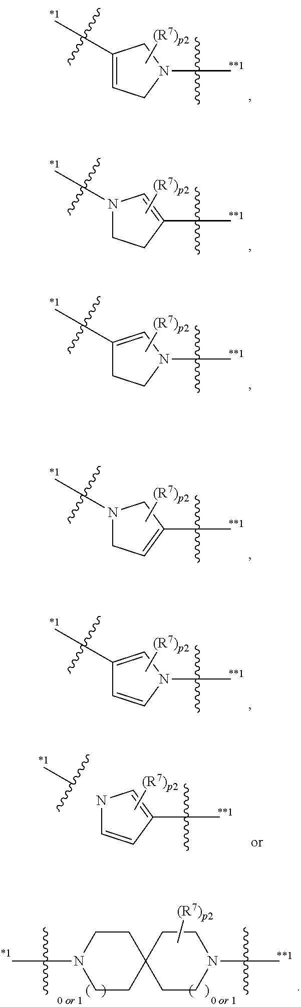

6. The compound according to claim 1, wherein Cy1 is selected from

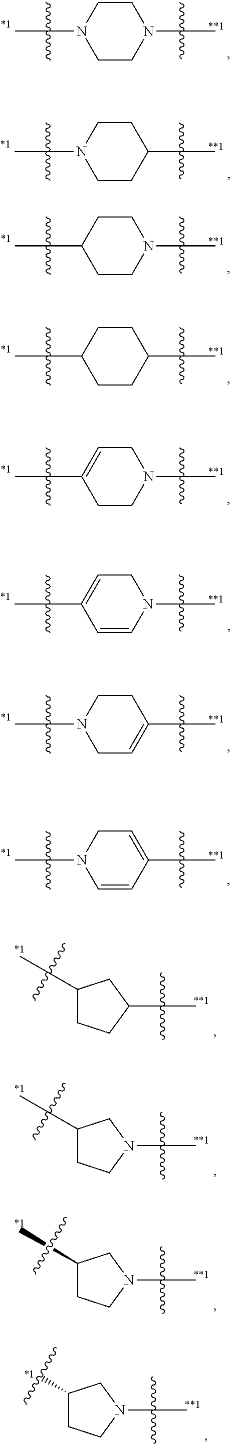

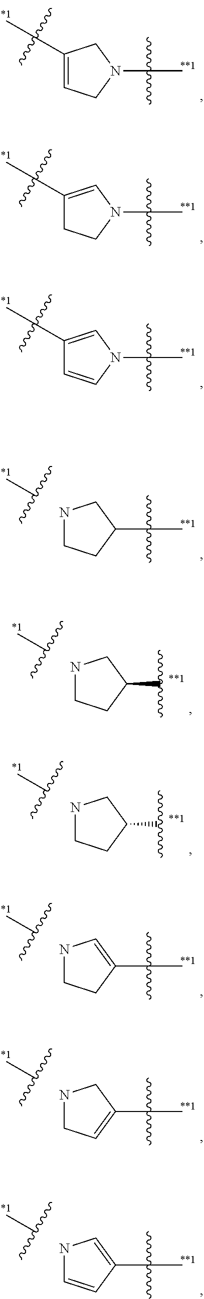

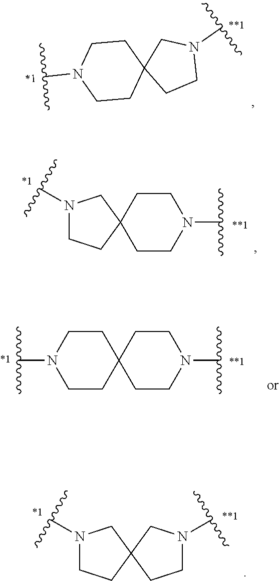 [SMILES: *N1CCC2(CC1)CCN(*)C2 ; *N1CCC2(CC1)CCN(*)C2 ; *N1CCC2(CC1)CCN(*)CC2 ; *N1CCC2(CCN(*)C2)C1]

7. The compound according to claim 1, wherein p2 is 0, 1, or 2, and each R⁷ is independently selected hydrogen, —F, —Br, —Cl, —I, —CH₃, —C₂H₅, —C₃H₇, —C₄H₉, —C₅H₁₁, —C₆H₁₃, —C₇H₁₅, —C₈H₁₇, —OCH₃, —OC₂H₅, —OC₃H₇, —OC₄H₉, —OC₅H₁₁, —OC₆H₁₃, —OC₇H₁₅, —OC₈H₁₇, —C₂₋₈ alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —CH₃, —C₂H₅, —C₃H₇, —C₄H₉, —C₅H₁₁, —C₆H₁₃, —C₇H₁₅, —C₈H₁₇, —OCH₃, —OC₂H₅, —OC₃H₇, —OC₄H₉, —OC₅H₁₁, —OC₆H₁₃, —OC₇H₁₅, —OC₈H₁₇, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, is optionally substituted with —F, —Br, —Cl, —I, hydroxy, -haloC₁₋₈alkyl, —C₁₋₈alkyoxy, cycloalkyl, heterocyclyl, aryl, or heteroaryl;
R^{a}, R^{b} and R^{c} are defined as claim 1.

8. The compound according to claim 1, wherein p2 is 0, 1, or 2, and each R⁷ is independently selected from hydrogen, —F, —Br, —Cl, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl or cyclohexyl.

9. The compound according to claim 1, wherein the [symbol] moiety is selected from 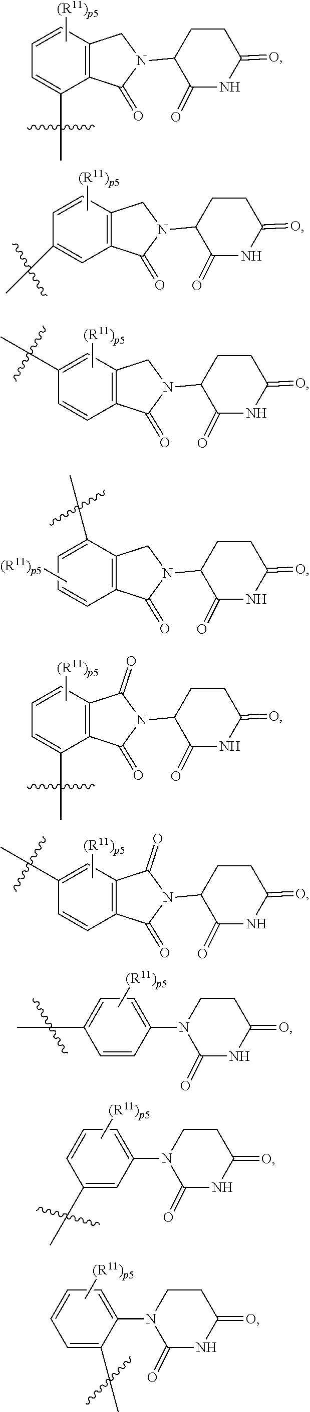 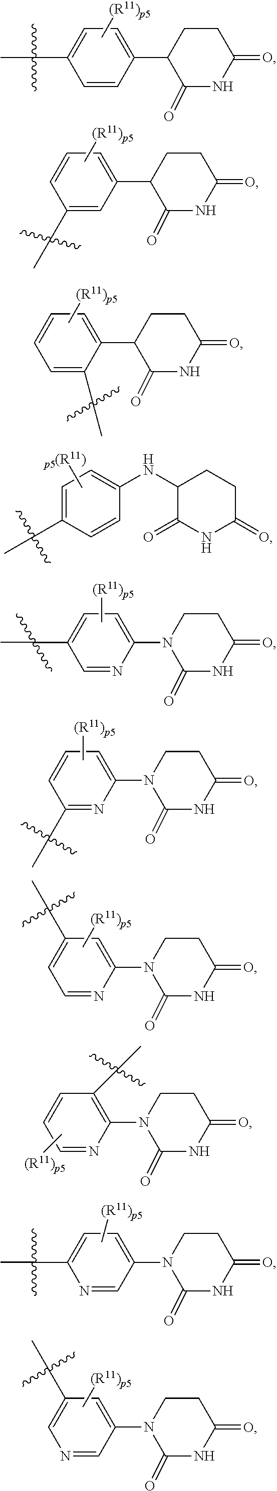 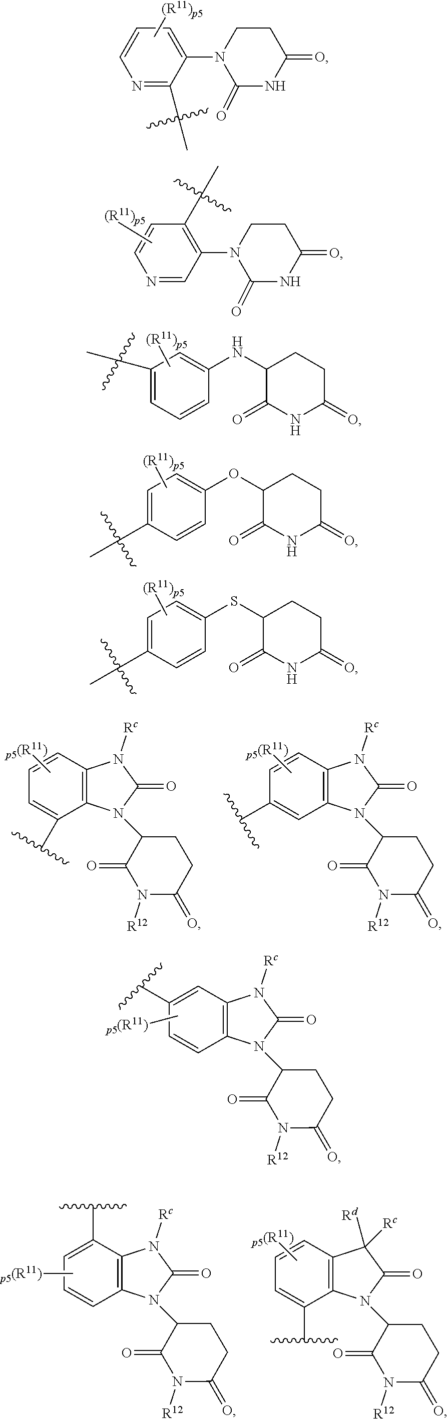 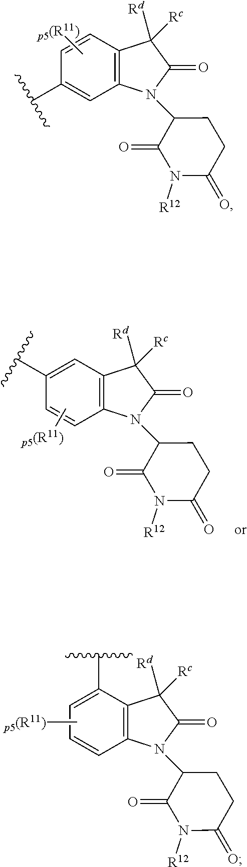
R¹¹ is selected from hydrogen, —F, —Br, —Cl, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl or cyclohexyl;
R^{c} and R^{d} are each independently hydrogen, —CH₃, —C₂H₅, —C₃H₇, —C₄H₉, —CH, —C₆H₁₃, —C₂₋₈ alkenyl, —C₂₋₈alkynyl, cyclopropyl, cyclobutyl, cyclopentyl, cyclohexyl, heterocyclyl, aryl, or heteroaryl; or
R^{c} and R^{d} together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂,
and p5 is 0 or 1.

10. The compound according to claim 6, wherein the [symbol] moiety is selected from

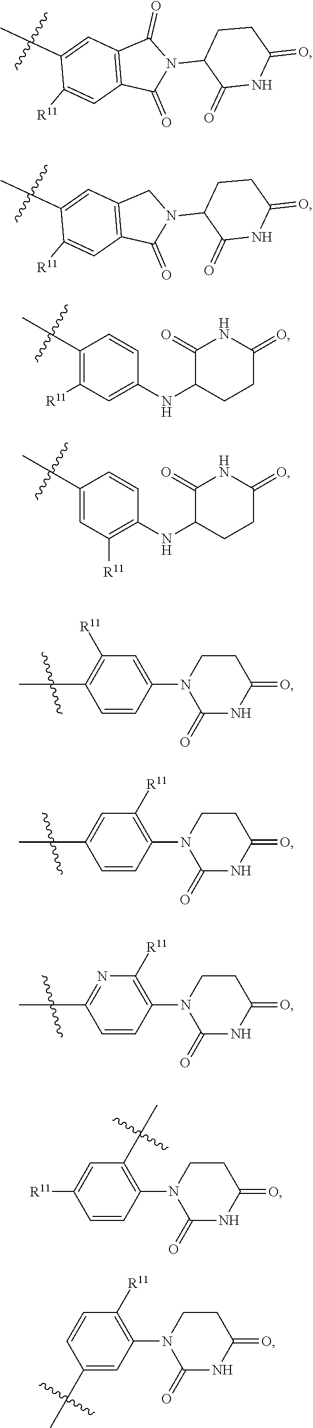

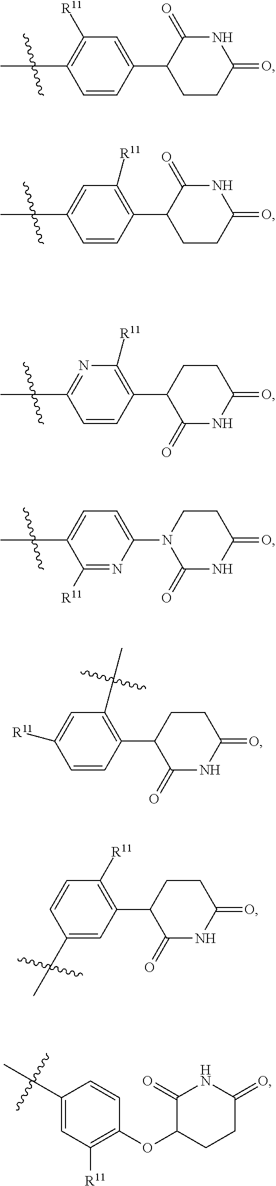

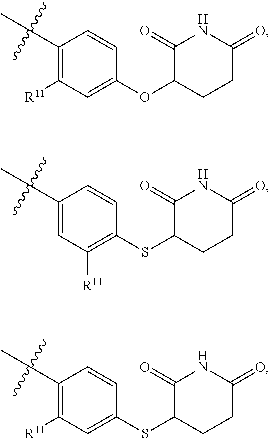

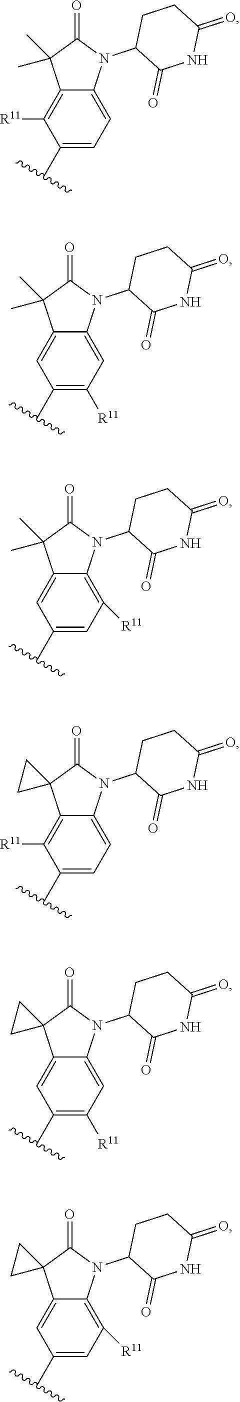

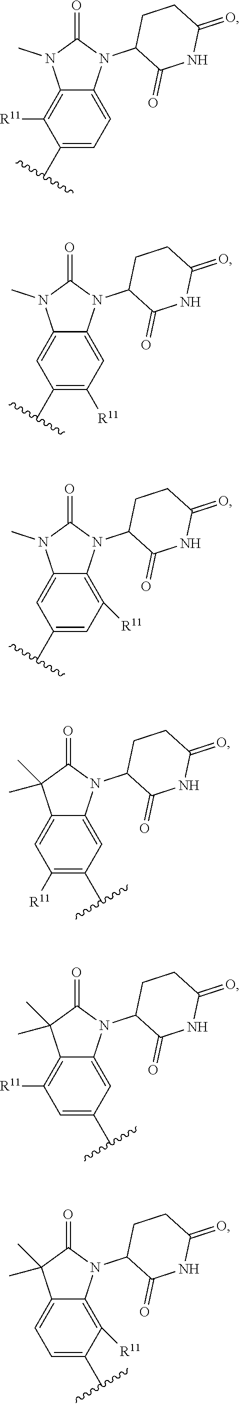

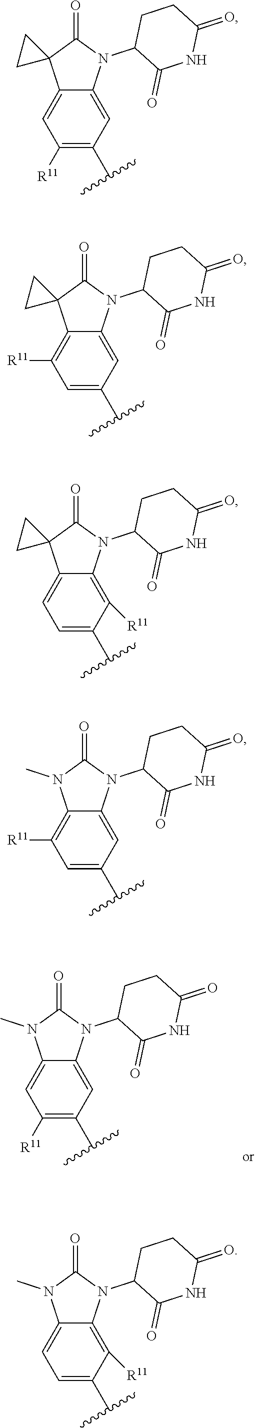

11. The compound according to claim 6, wherein the [symbol] moiety is selected from

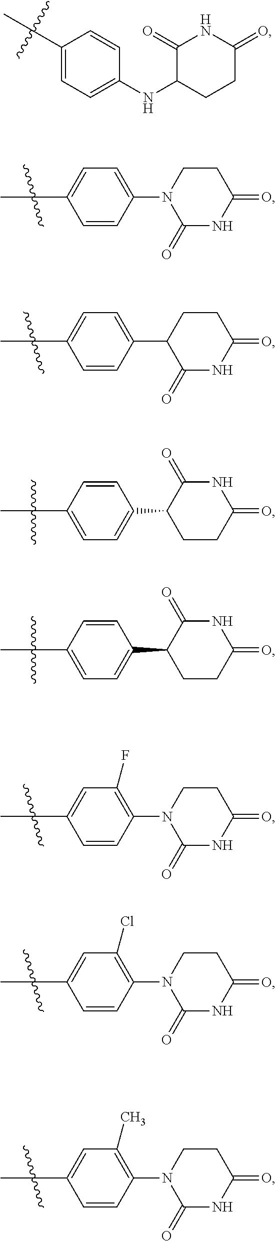

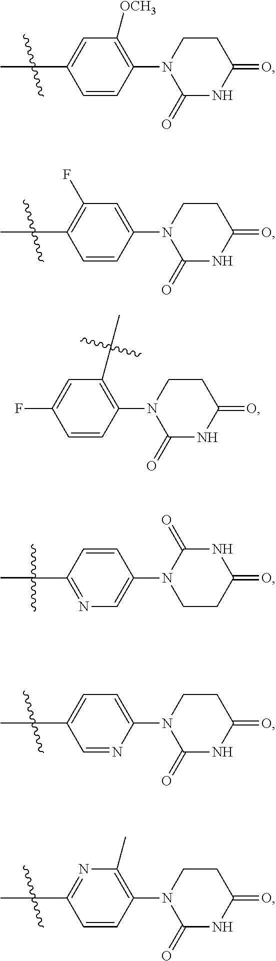 [SMILES: *c1ccc(N2CCC(=O)NC2=O)c(OC)c1 ; *c1ccc(N2CCC(=O)NC2=O)cc1F ; *c1cc(F)ccc1N1CCC(=O)NC1=O ; *c1ccc(N2CCC(=O)NC2=O)cn1 ; *c1ccc(N2CCC(=O)NC2=O)nc1 ; *c1ccc(N2CCC(=O)NC2=O)c(C)n1]

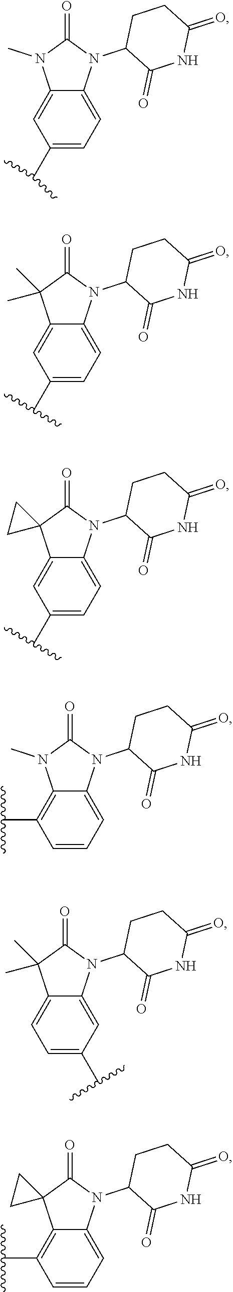 [SMILES: *c1ccc2c(c1)n(C)c(=O)n2C1CCC(=O)NC1=O ; *c1ccc2c(c1)C(C)(C)C(=O)N2C1CCC(=O)NC1=O ; *c1ccc2c(c1)C1(CC1)C(=O)N2C1CCC(=O)NC1=O ; *c1cccc2c1n(C)c(=O)n2C1CCC(=O)NC1=O ; *c1ccc2c(c1)N(C1CCC(=O)NC1=O)C(=O)C2(C)C ; *c1cccc2c1C1(CC1)C(=O)N2C1CCC(=O)NC1=O]

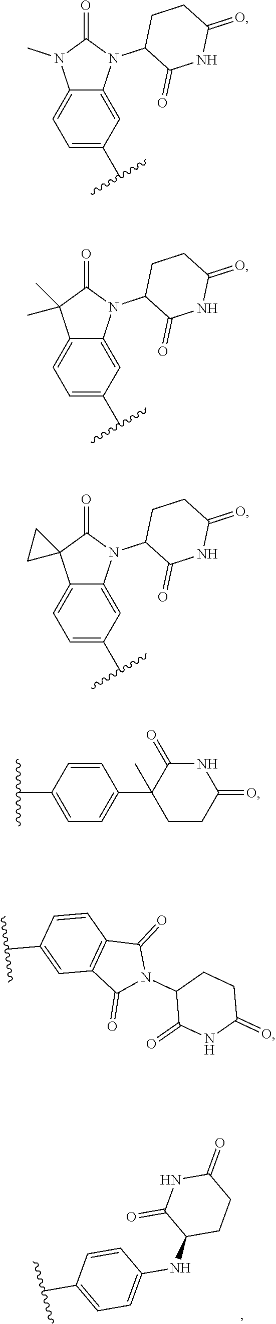 [SMILES: *c1ccc2c(c1)n(C1CCC(=O)NC1=O)c(=O)n2C ; *c1ccc2c(c1)N(C1CCC(=O)NC1=O)C(=O)C2(C)C ; *c1ccc2c(c1)N(C1CCC(=O)NC1=O)C(=O)C21CC1 ; *c1ccc(C2(C)CCC(=O)NC2=O)cc1 ; *c1ccc2c(c1)C(=O)N(C1CCC(=O)NC1=O)C2=O ; *c1ccc(N[C@@H]2CCC(=O)NC2=O)cc1]

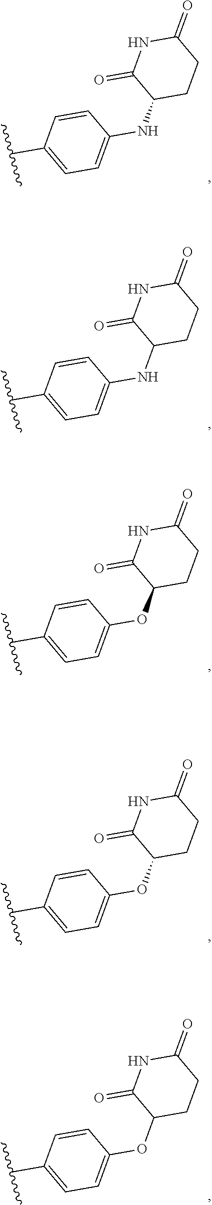 [SMILES: *c1ccc(N[C@H]2CCC(=O)NC2=O)cc1 ; *c1ccc(NC2CCC(=O)NC2=O)cc1 ; *c1ccc(O[C@@H]2CCC(=O)NC2=O)cc1 ; *c1ccc(O[C@H]2CCC(=O)NC2=O)cc1 ; *c1ccc(OC2CCC(=O)NC2=O)cc1]

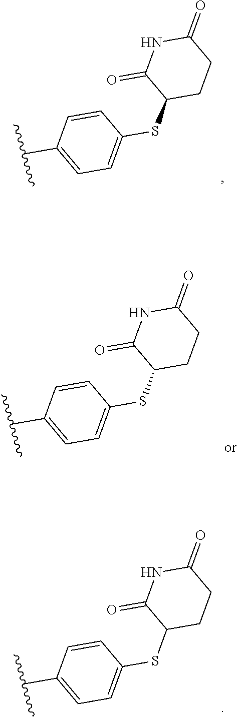 [SMILES: *c1ccc(S[C@@H]2CCC(=O)NC2=O)cc1 ; *c1ccc(S[C@H]2CCC(=O)NC2=O)cc1 ; *c1ccc(SC2CCC(=O)NC2=O)cc1]

12. The compound according to claim 1, wherein L^{a}, L^{b} and L^{c} are each independently selected from a single bond, —O—, —S—, —C(O)—, —(CR^{c}R^{d})ₙ— or —NR^{c}—;
n is 1, 2, 3, 4, 5 or 6;
R^{c} and R^{d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl or cyclohexyl, cycloheptyl, cyclooctyl, heterocyclyl, aryl, or heteroaryl; or
R^{c} and R^{d} together with the atom(s) to which they are attached, form a 3-, 4-, 5-, 6-, 7-, 8-, 9-, 10-, 11- or 12-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, oxo, —CN or —NO₂.

13. The compound according to claim 1, wherein L^{a}, L^{b} and L^{c} are each independently selected from a single bond, —O—, —CO—, —(CH₂)ₙ—, —[CH(CH₃)]ₙ—, —[C(CH₃)₂]ₙ— or —NH—; n is 1, 2, 3, 4 or 5.

14. The compound according to claim 1, R¹³ is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl.

15. The compound according to claim 1, wherein m1, m2, m3 and m4 are each independently selected from 0 or 1.

16. The compound according to claim 1, wherein p3 and p4 are each independently 0 or 1, R⁸ and R⁹ are each independently selected from hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl.

17. The compound according to claim 1, wherein the 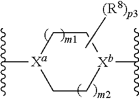
moiety is selected from 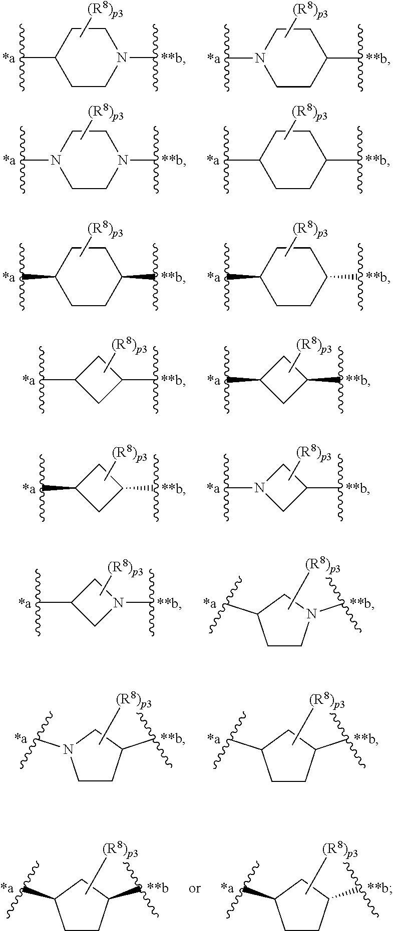
wherein *a refers to the position attached to the 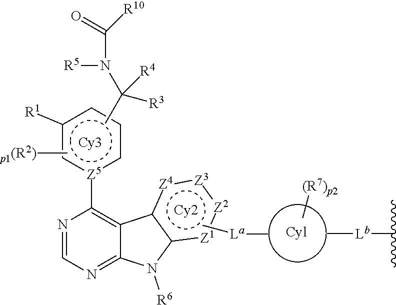
moiety, and **b refers to the position attached to 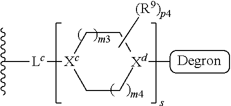
moiety.

18. The compound according to claim 1, wherein the 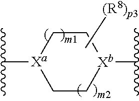
moiety is selected from 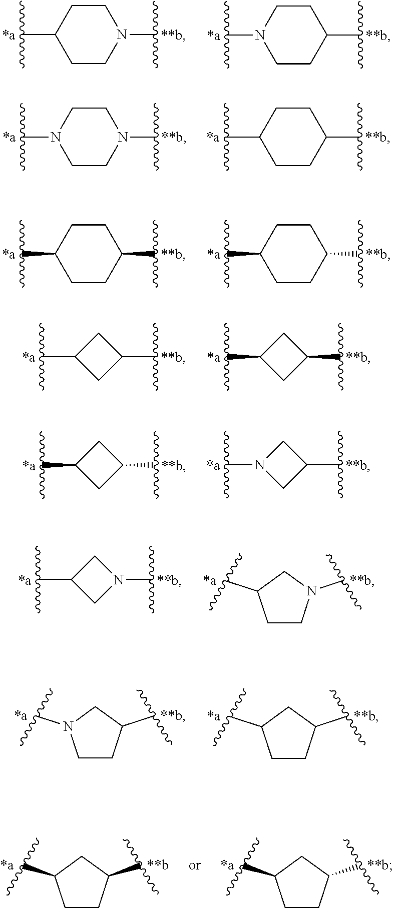
wherein *a refers to the position attached to the 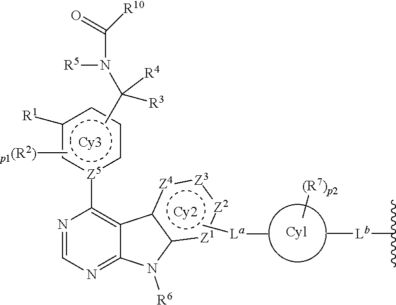
moiety, and **b refers to the position attached to 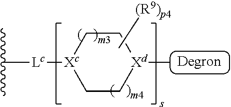
moiety.

19. The compound according to claim 1, wherein the 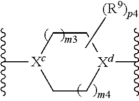
moiety is selected from 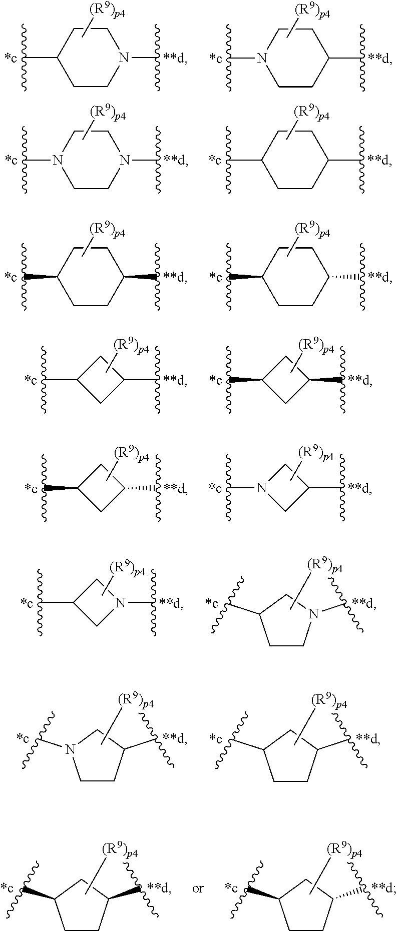
wherein *a refers to the position attached to the 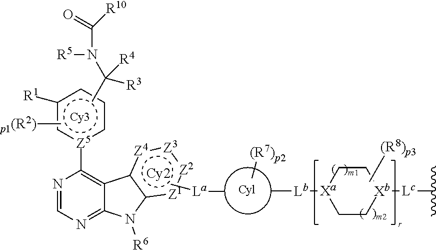
moiety, and **b refers to the position attached to 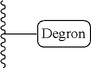
moiety.

20. The compound according to claim 1, wherein the 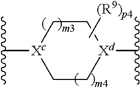
moiety is selected from 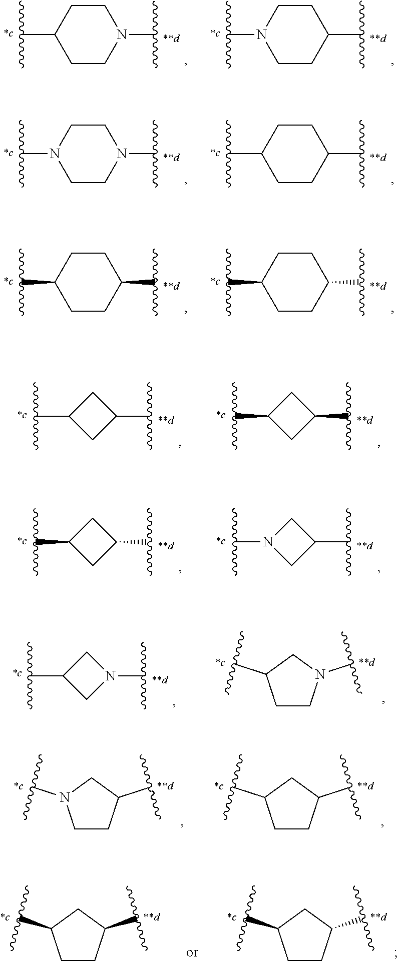
wherein *a refers to the position attached to the 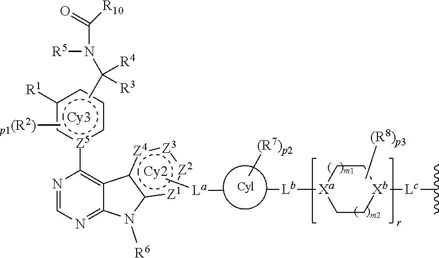
moiety, and **b refers to the position attached to 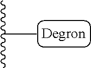
moiety.

21. The compound according to claim 1, wherein the 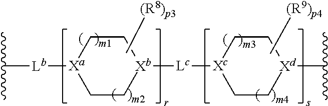
moiety is selected from —CH₂—, —(CH₂)₂—, —(CH₂)₃—, —(C₂)₄—, 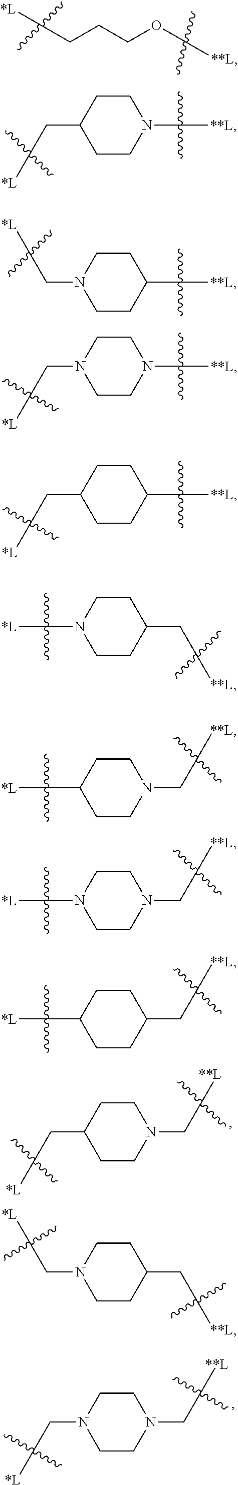 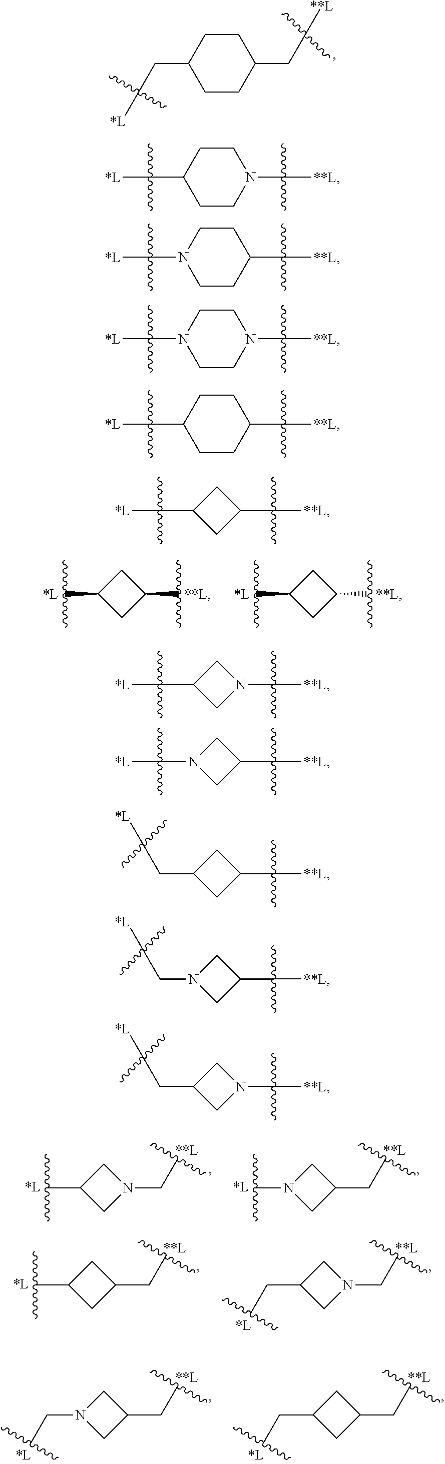 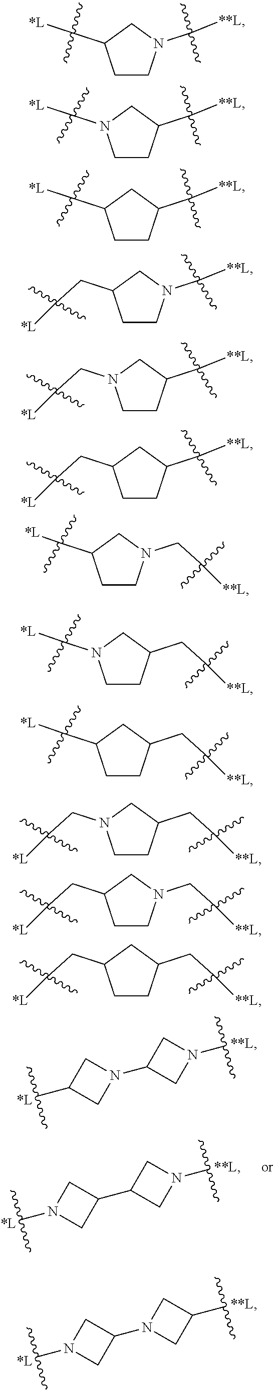
*L refers to the position attached to the 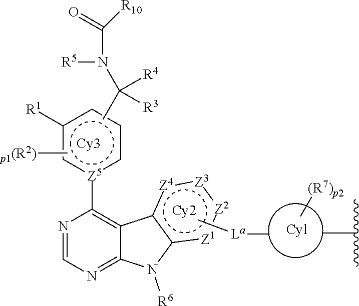
moiety, and **L refers to the position attached to 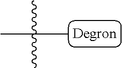
moiety.

22. The compound according to claim 1, wherein Z¹ and Z⁴ are each independently CR^{za} or N; R^{za} is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl, —C₂₋₈alkenyl, —C₂₋₈alkynyl, heterocyclyl, aryl, heteroaryl, —CN.

23. The compound according to claim 1, wherein one of Z² and Z³ is C, and connects to Cy1; and the other one of Z² and Z³ is CR^{za} or N; R^{za} is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl, —C₂₋₈alkenyl, —C₂₋₈alkynyl, heterocyclyl, aryl, heteroaryl, —CN.

24. The compound according to claim 1, wherein at most two of Z¹, Z², Z³ and Z⁴ are N.

25. The compound according to claim 1, wherein at most one of Z¹, Z², Z³ and Z⁴ is N.

26. The compound according to claim 1, wherein R¹ is hydrogen, —F, —Cl, —Br, —I, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃ or —CH₂CH₂CF₃.

27. The compound according to claim 1, wherein R¹ and R³, together with the atom(s) to which they are attached, form a 3-, 4-, 5-, 6-, 7- or 8-membered ring, said ring comprising 0, 1 or 2 additional N atom as ring member(s), said ring is optionally substituted with at least one substituent independently selected from —F, —Cl, —Br, —I, —OH, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or —CH₂OH.

28. The compound according to claim 1, wherein R² is H, —F, —Cl, —Br, —I, methyl, ethyl or propyl, and p1 is 0, 1, or 2.

29. The compound according to claim 1, wherein R³ and R⁴ are each independently —H, methyl, ethyl, propyl, butyl, pentyl, hexyl, cyclopropyl, cyclobutyl, cyclopentyl or cyclohexyl.

30. The compound according to claim 1, wherein the 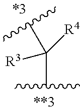
moiety is 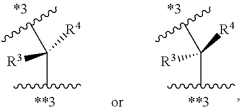
wherein *3 refers to the position attached to the 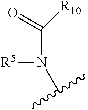
moiety, and **3 refers to the position attached to 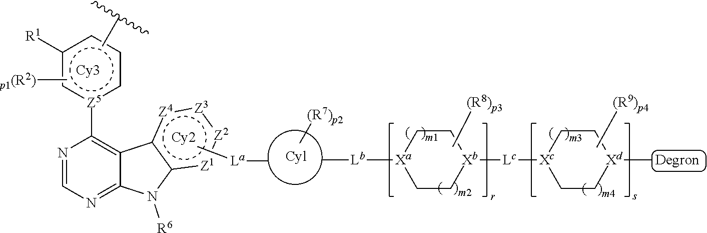
moiety.

31. The compound according to claim 1, wherein R⁵ and R⁶ are each independently —H, methyl, ethyl, propyl.

32. The compound according to claim 1, wherein R¹⁰ is 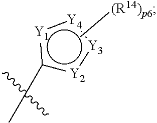
Y¹, Y², Y³ and Y⁴ are selected from CH, O, S or N; R¹⁴ is selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₁₋₈alkoxy, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{a}, R^{b}, and R^{c} are each independently hydrogen, —C₁₋₈alkyl, —C₂₋₈ alkenyl, —C₂₋₈alkynyl, cycloalkyl, heterocyclyl, aryl, or heteroaryl; and p6 is 0, 1, 2, 3 or 4.

33. The compound according to claim 25, wherein Y¹ is CH, S, N or O; Y² is CH, S, O or N; Y³ is CH, O, S or N; and Y⁴ is CH, O, S or N.

34. The compound according to claim 25, wherein 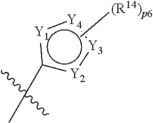
is selected from

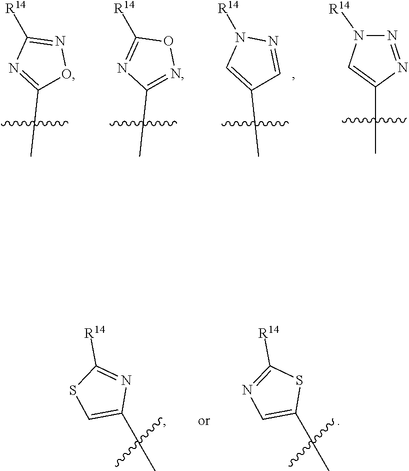

35. The compound according to claim 25, wherein, R¹⁴ is selected from —H, —F, —Cl, —Br, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃, —CH₂CH₂CF₃, each of said —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl is optionally substituted with at least one halogen, hydroxy, -haloC₁₋₈alkyl, —C₁₋₈alkyoxy, cycloalkyl, heterocyclyl, aryl, or heteroaryl.

36. The compound according to claim 25, wherein, R¹⁴ is selected from

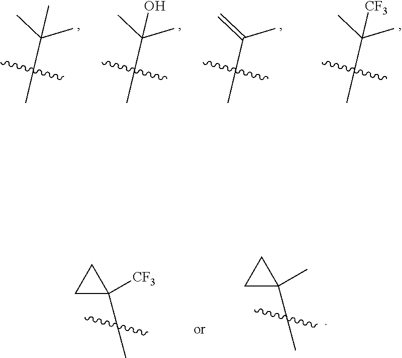

37. The compound according to claim 1, wherein the compound is selected from

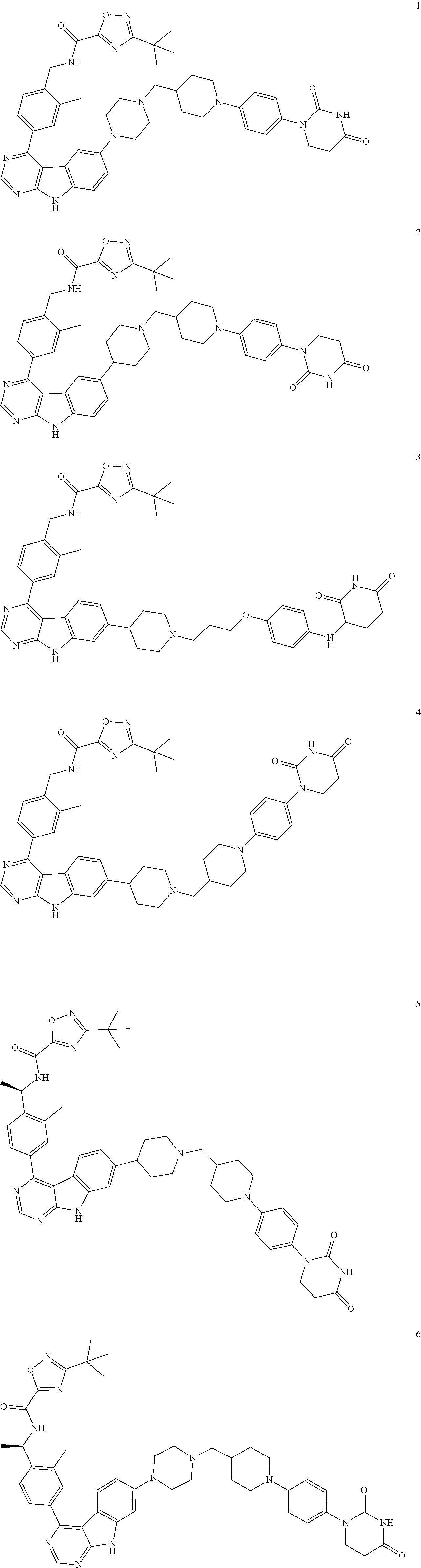

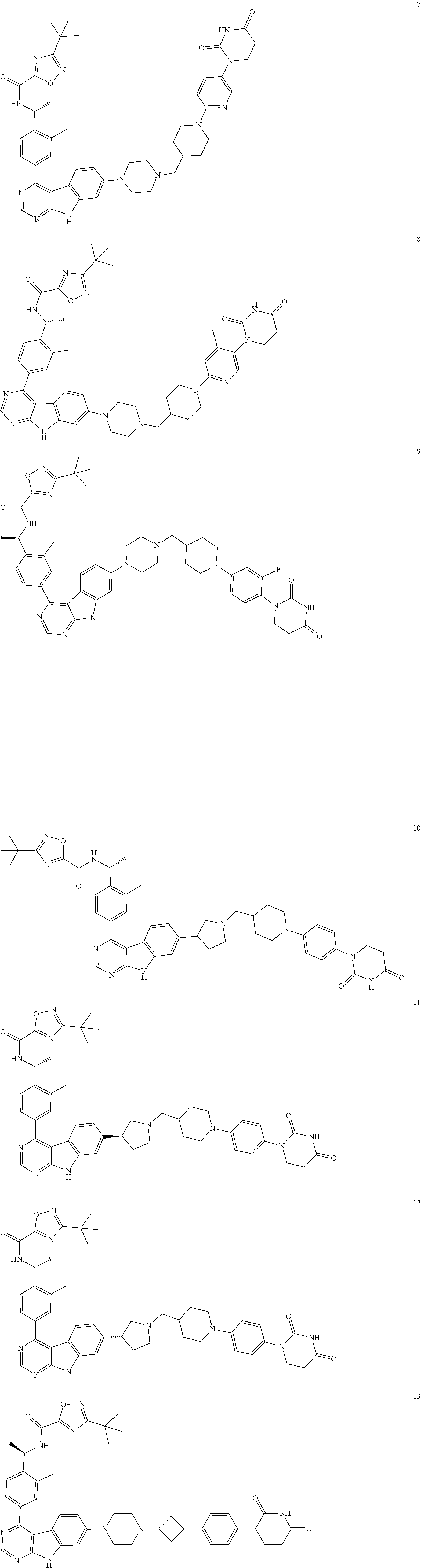

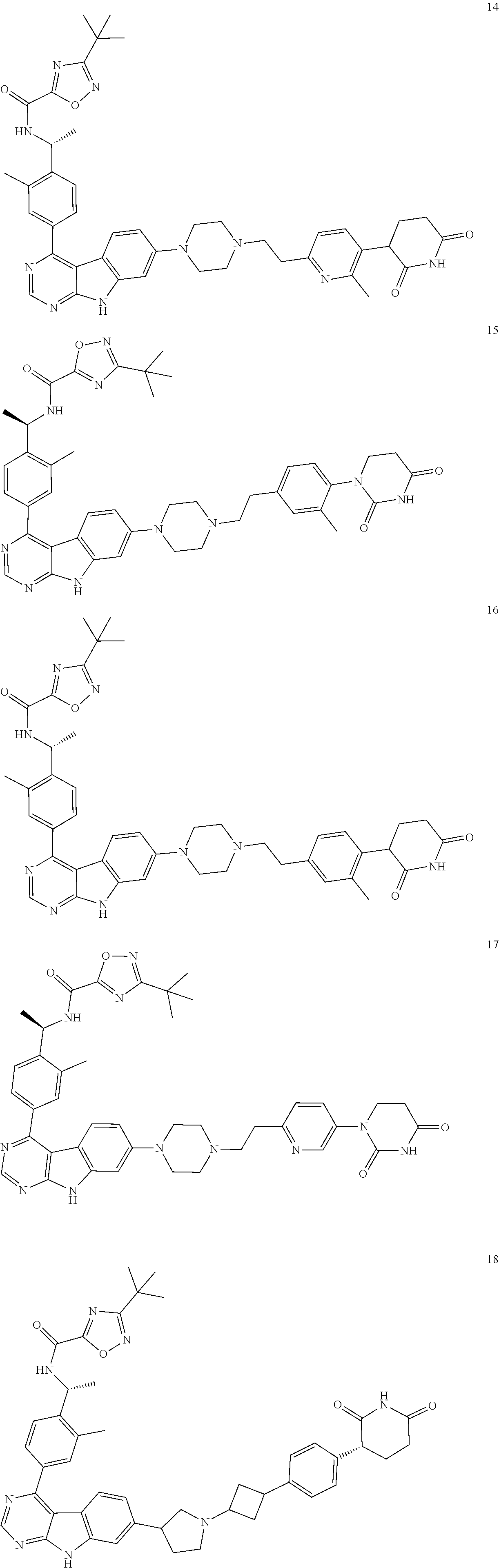

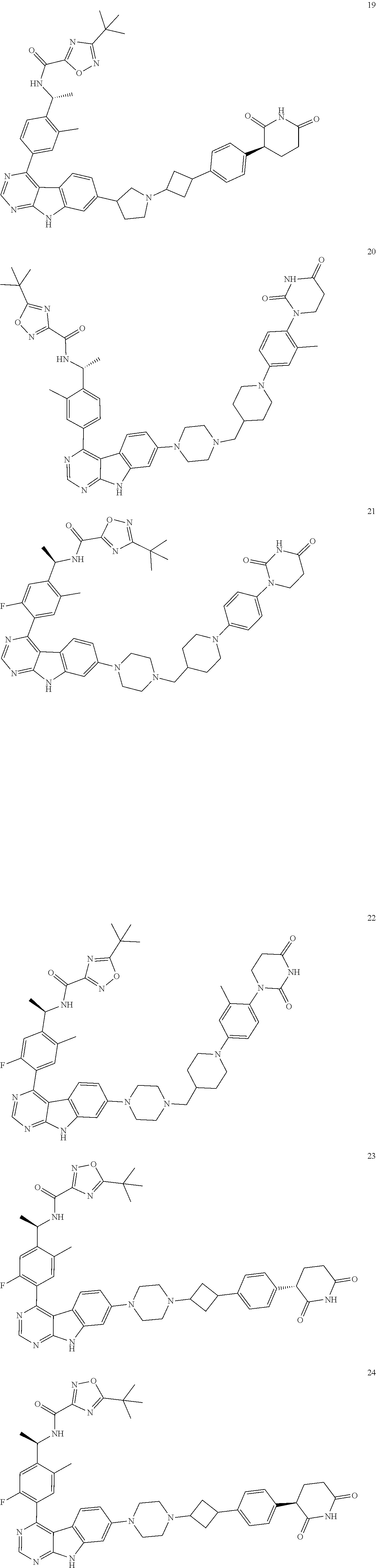

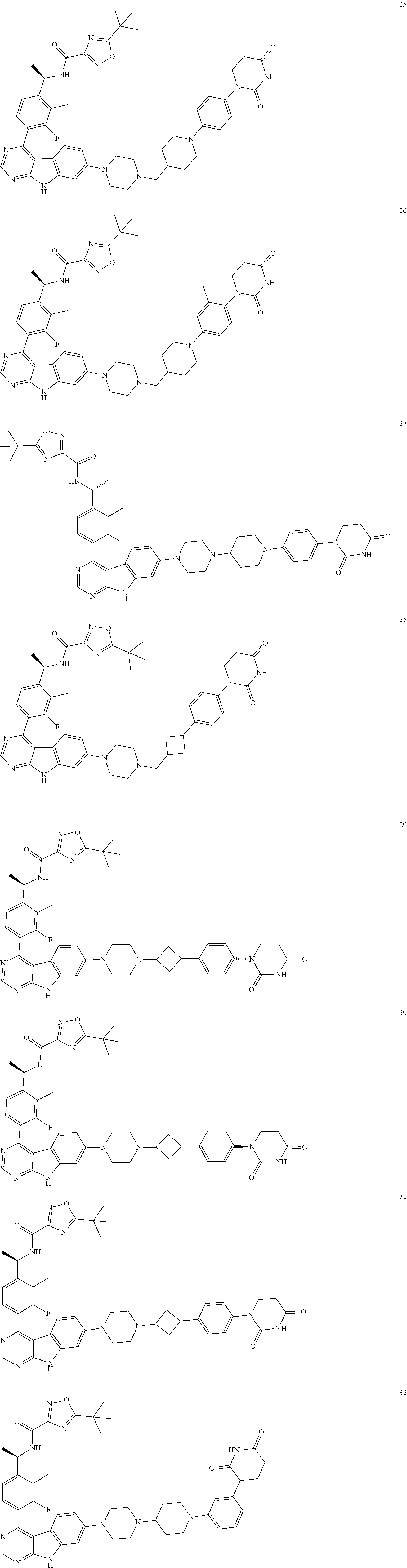

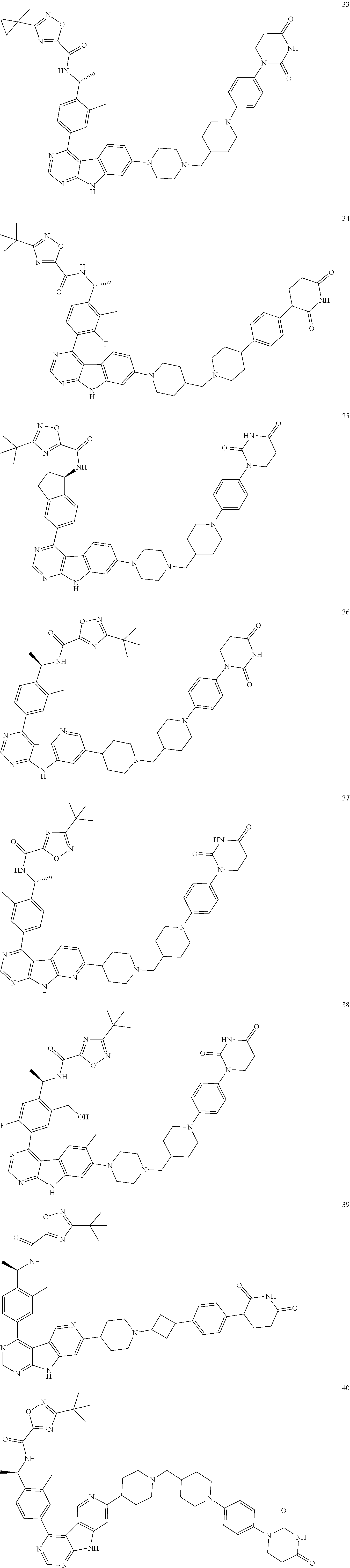

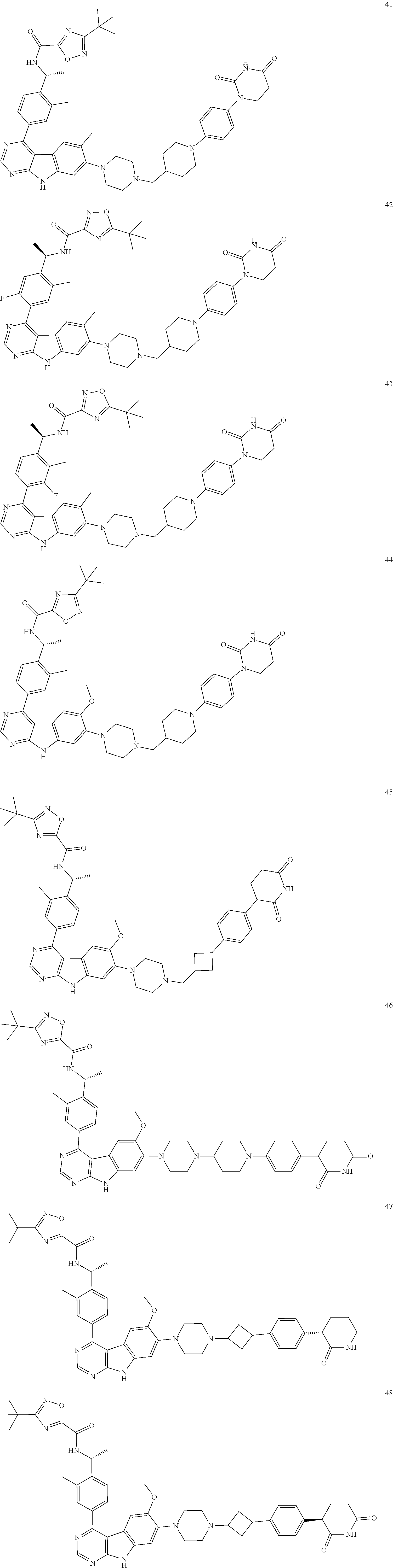

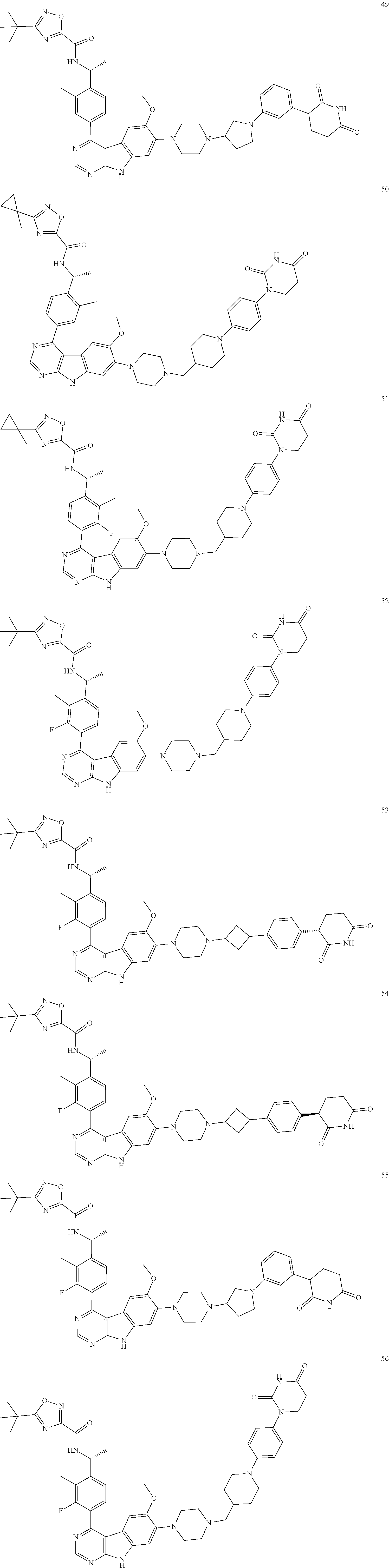

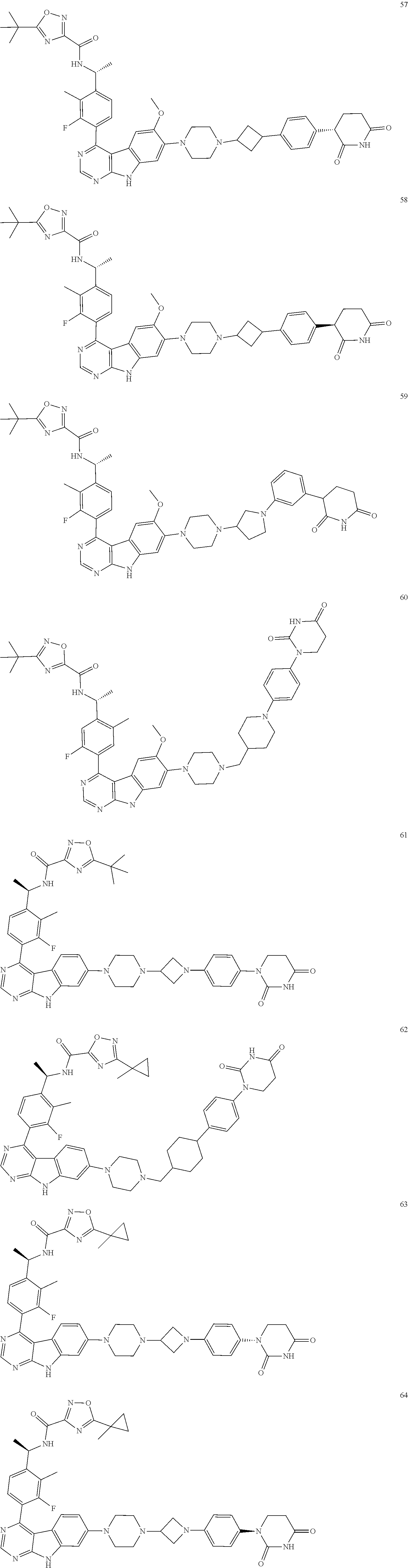

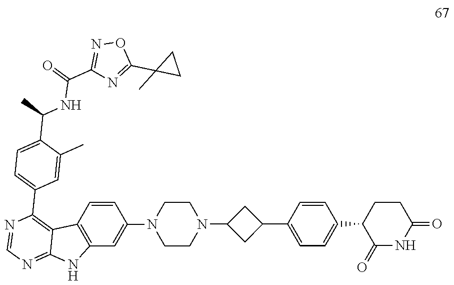 [SMILES: Cc1cc(-c2ncnc3[nH]c4cc(N5CCN(C6CC(c7ccc([C@@H]8CCC(=O)NC8=O)cc7)C6)CC5)ccc4c23)ccc1[C@@H](C)NC(=O)c1noc(C2(C)CC2)n1]

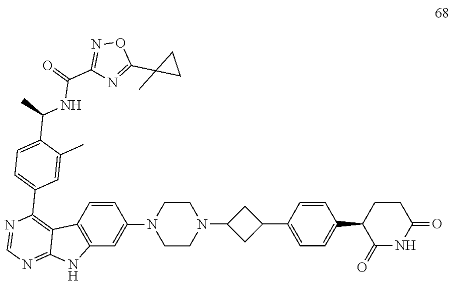 [SMILES: Cc1cc(-c2ncnc3[nH]c4cc(N5CCN(C6CC(c7ccc([C@H]8CCC(=O)NC8=O)cc7)C6)CC5)ccc4c23)ccc1[C@@H](C)NC(=O)c1noc(C2(C)CC2)n1]

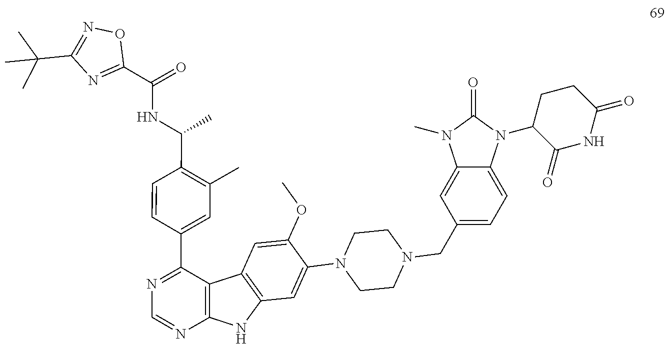 [SMILES: COc1cc2c(cc1N1CCN(Cc3ccc4c(c3)n(C)c(=O)n4C3CCC(=O)NC3=O)CC1)[nH]c1ncnc(-c3ccc([C@@H](C)NC(=O)c4nc(C(C)(C)C)no4)c(C)c3)c12]

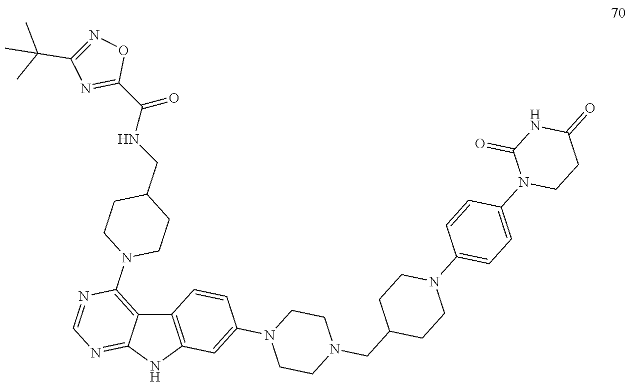 [SMILES: CC(C)(C)c1noc(C(=O)NCC2CCN(c3ncnc4[nH]c5cc(N6CCN(CC7CCN(c8ccc(N9CCC(=O)NC9=O)cc8)CC7)CC6)ccc5c34)CC2)n1]

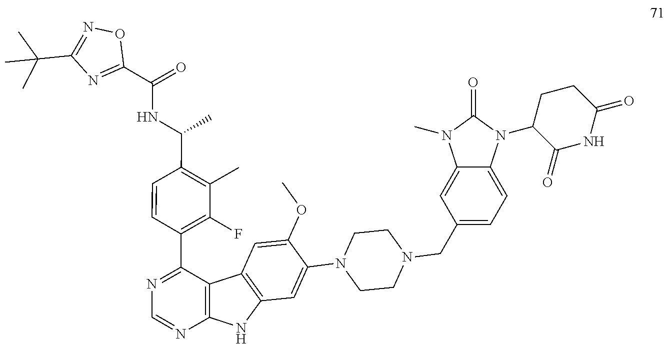 [SMILES: COc1cc2c(cc1N1CCN(Cc3ccc4c(c3)n(C)c(=O)n4C3CCC(=O)NC3=O)CC1)[nH]c1ncnc(-c3ccc([C@@H](C)NC(=O)c4nc(C(C)(C)C)no4)c(C)c3F)c12]

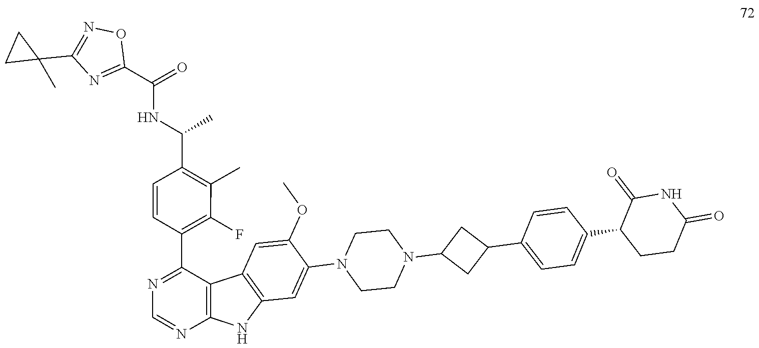 [SMILES: COc1cc2c(cc1N1CCN(C3CC(c4ccc([C@H]5CCC(=O)NC5=O)cc4)C3)CC1)[nH]c1ncnc(-c3ccc([C@@H](C)NC(=O)c4nc(C5(C)CC5)no4)c(C)c3F)c12]

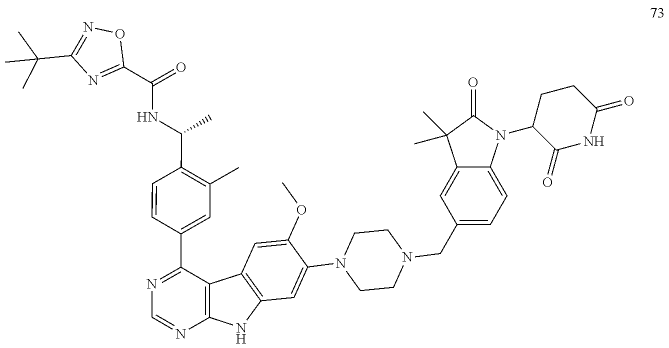 [SMILES: COc1cc2c(cc1N1CCN(Cc3ccc4c(c3)C(C)(C)C(=O)N4C3CCC(=O)NC3=O)CC1)[nH]c1ncnc(-c3ccc([C@@H](C)NC(=O)c4nc(C(C)(C)C)no4)c(C)c3)c12]

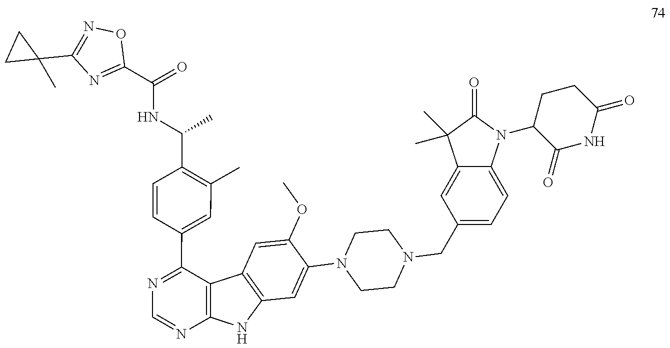 [SMILES: COc1cc2c(cc1N1CCN(Cc3ccc4c(c3)C(C)(C)C(=O)N4C3CCC(=O)NC3=O)CC1)[nH]c1ncnc(-c3ccc([C@@H](C)NC(=O)c4nc(C5(C)CC5)no4)c(C)c3)c12]

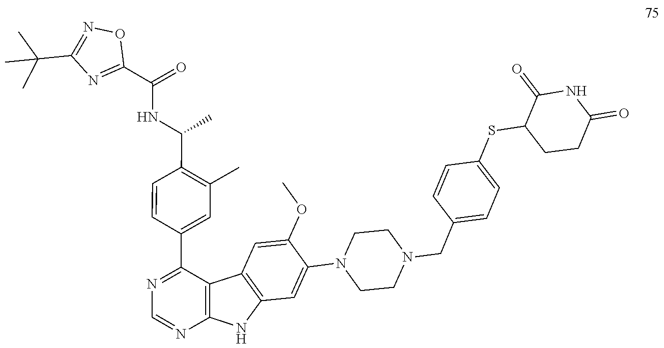 [SMILES: COc1cc2c(cc1N1CCN(Cc3ccc(SC4CCC(=O)NC4=O)cc3)CC1)[nH]c1ncnc(-c3ccc([C@@H](C)NC(=O)c4nc(C(C)(C)C)no4)c(C)c3)c12]

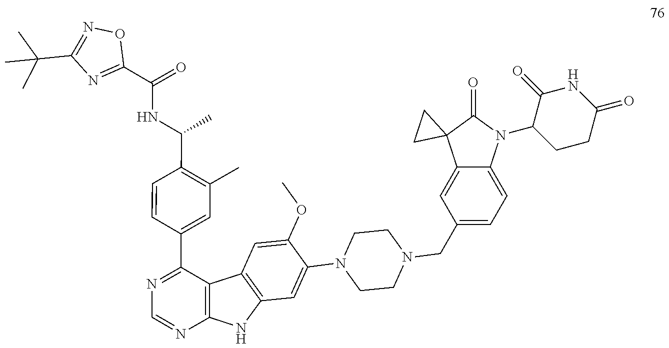 [SMILES: COc1cc2c(cc1N1CCN(Cc3ccc4c(c3)C3(CC3)C(=O)N4C3CCC(=O)NC3=O)CC1)[nH]c1ncnc(-c3ccc([C@@H](C)NC(=O)c4nc(C(C)(C)C)no4)c(C)c3)c12]

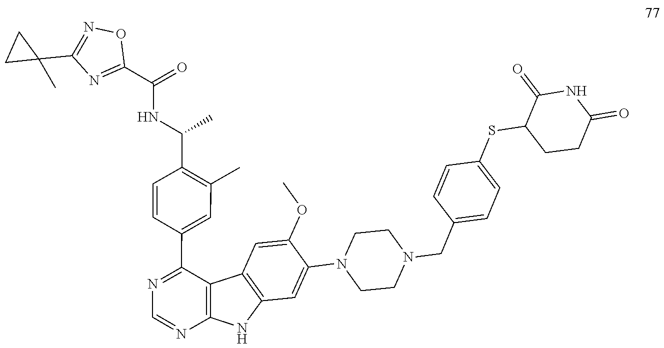 [SMILES: COc1cc2c(cc1N1CCN(Cc3ccc(SC4CCC(=O)NC4=O)cc3)CC1)[nH]c1ncnc(-c3ccc([C@@H](C)NC(=O)c4nc(C5(C)CC5)no4)c(C)c3)c12]

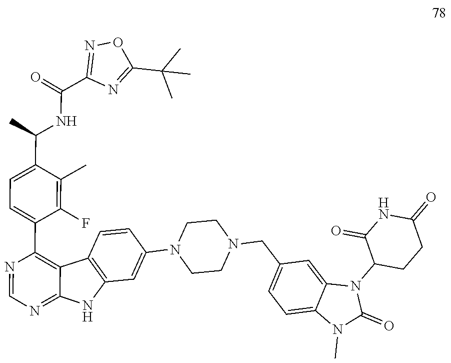 [SMILES: Cc1c([C@@H](C)NC(=O)c2noc(C(C)(C)C)n2)ccc(-c2ncnc3[nH]c4cc(N5CCN(Cc6ccc7c(c6)n(C6CCC(=O)NC6=O)c(=O)n7C)CC5)ccc4c23)c1F]

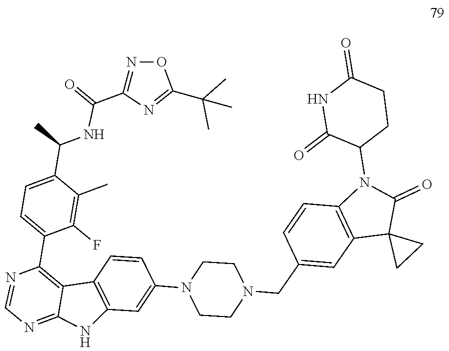 [SMILES: Cc1c([C@@H](C)NC(=O)c2noc(C(C)(C)C)n2)ccc(-c2ncnc3[nH]c4cc(N5CCN(Cc6ccc7c(c6)C6(CC6)C(=O)N7C6CCC(=O)NC6=O)CC5)ccc4c23)c1F]

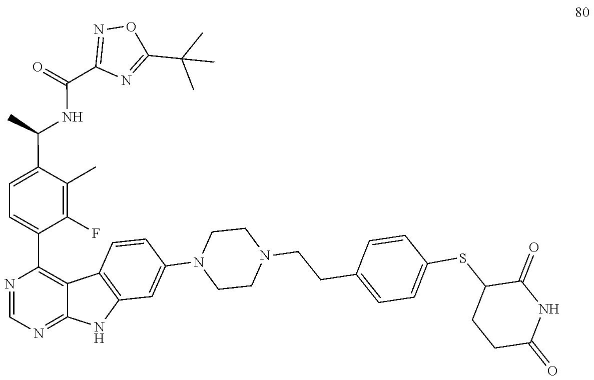 [SMILES: Cc1c([C@@H](C)NC(=O)c2noc(C(C)(C)C)n2)ccc(-c2ncnc3[nH]c4cc(N5CCN(CCc6ccc(SC7CCC(=O)NC7=O)cc6)CC5)ccc4c23)c1F]

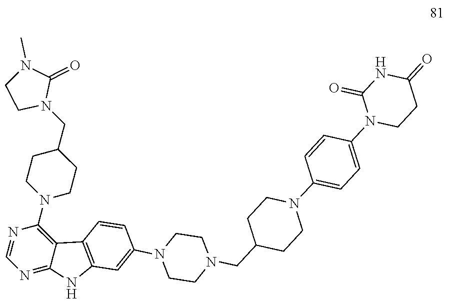 [SMILES: CN1CCN(CC2CCN(c3ncnc4[nH]c5cc(N6CCN(CC7CCN(c8ccc(N9CCC(=O)NC9=O)cc8)CC7)CC6)ccc5c34)CC2)C1=O]

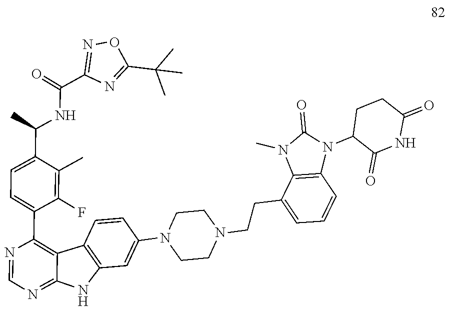 [SMILES: Cc1c([C@@H](C)NC(=O)c2noc(C(C)(C)C)n2)ccc(-c2ncnc3[nH]c4cc(N5CCN(CCc6cccc7c6n(C)c(=O)n7C6CCC(=O)NC6=O)CC5)ccc4c23)c1F]

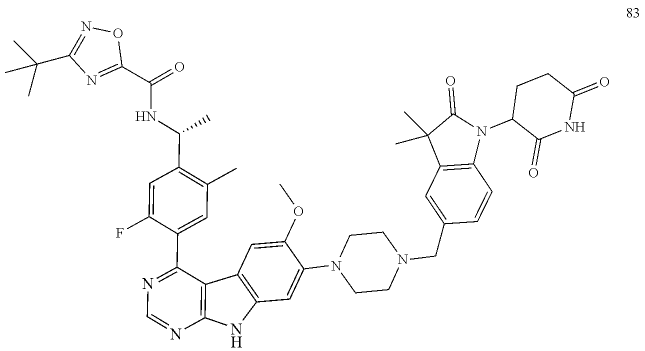 [SMILES: COc1cc2c(cc1N1CCN(Cc3ccc4c(c3)C(C)(C)C(=O)N4C3CCC(=O)NC3=O)CC1)[nH]c1ncnc(-c3cc(C)c([C@@H](C)NC(=O)c4nc(C(C)(C)C)no4)cc3F)c12]

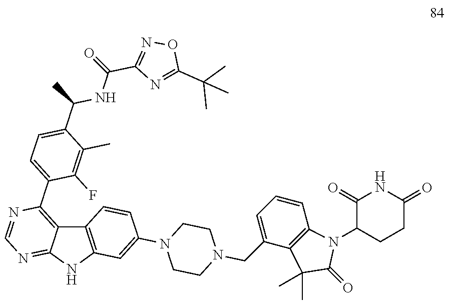 [SMILES: Cc1c([C@@H](C)NC(=O)c2noc(C(C)(C)C)n2)ccc(-c2ncnc3[nH]c4cc(N5CCN(Cc6cccc7c6C(C)(C)C(=O)N7C6CCC(=O)NC6=O)CC5)ccc4c23)c1F]

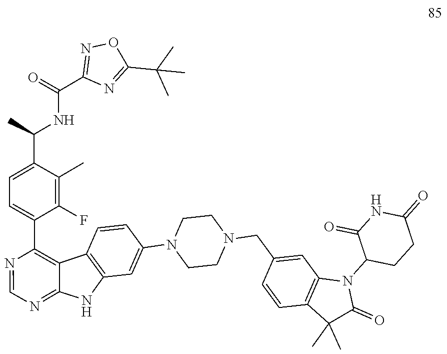 [SMILES: Cc1c([C@@H](C)NC(=O)c2noc(C(C)(C)C)n2)ccc(-c2ncnc3[nH]c4cc(N5CCN(Cc6ccc7c(c6)N(C6CCC(=O)NC6=O)C(=O)C7(C)C)CC5)ccc4c23)c1F]

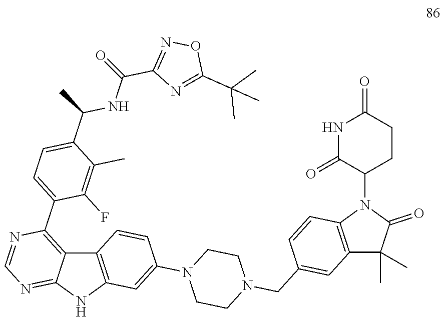 [SMILES: Cc1c([C@@H](C)NC(=O)c2noc(C(C)(C)C)n2)ccc(-c2ncnc3[nH]c4cc(N5CCN(Cc6ccc7c(c6)C(C)(C)C(=O)N7C6CCC(=O)NC6=O)CC5)ccc4c23)c1F]

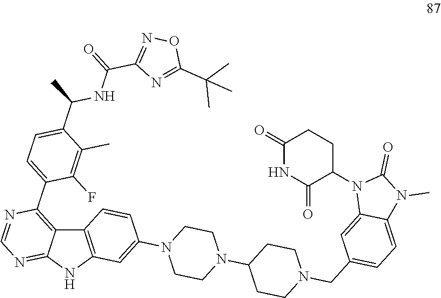 [SMILES: Cc1c([C@@H](C)NC(=O)c2noc(C(C)(C)C)n2)ccc(-c2ncnc3[nH]c4cc(N5CCN(C6CCN(Cc7ccc8c(c7)n(C7CCC(=O)NC7=O)c(=O)n8C)CC6)CC5)ccc4c23)c1F]

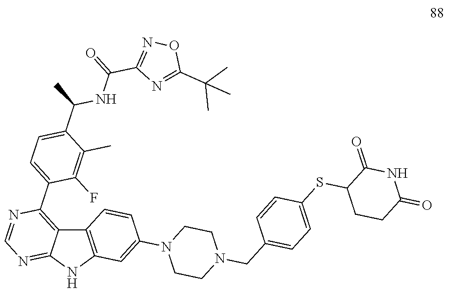 [SMILES: Cc1c([C@@H](C)NC(=O)c2noc(C(C)(C)C)n2)ccc(-c2ncnc3[nH]c4cc(N5CCN(Cc6ccc(SC7CCC(=O)NC7=O)cc6)CC5)ccc4c23)c1F]

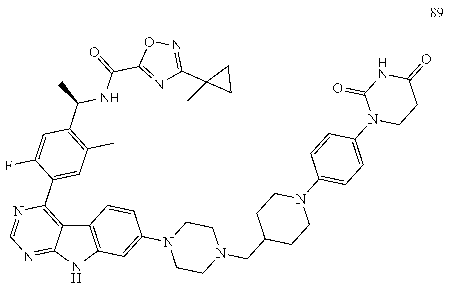 [SMILES: Cc1cc(-c2ncnc3[nH]c4cc(N5CCN(CC6CCN(c7ccc(N8CCC(=O)NC8=O)cc7)CC6)CC5)ccc4c23)c(F)cc1[C@@H](C)NC(=O)c1nc(C2(C)CC2)no1]

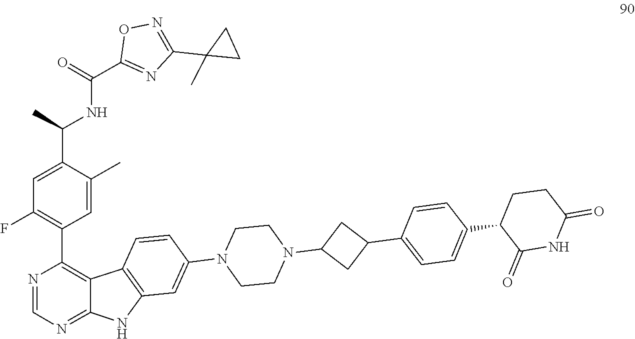 [SMILES: Cc1cc(-c2ncnc3[nH]c4cc(N5CCN(C6CC(c7ccc([C@@H]8CCC(=O)NC8=O)cc7)C6)CC5)ccc4c23)c(F)cc1[C@@H](C)NC(=O)c1nc(C2(C)CC2)no1]

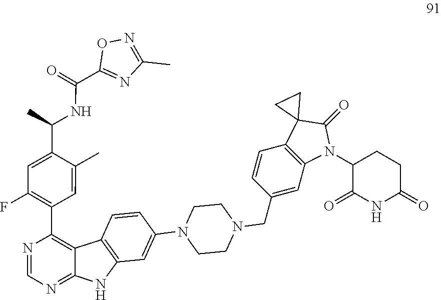 [SMILES: Cc1noc(C(=O)N[C@H](C)c2cc(F)c(-c3ncnc4[nH]c5cc(N6CCN(Cc7ccc8c(c7)N(C7CCC(=O)NC7=O)C(=O)C87CC7)CC6)ccc5c34)cc2C)n1]

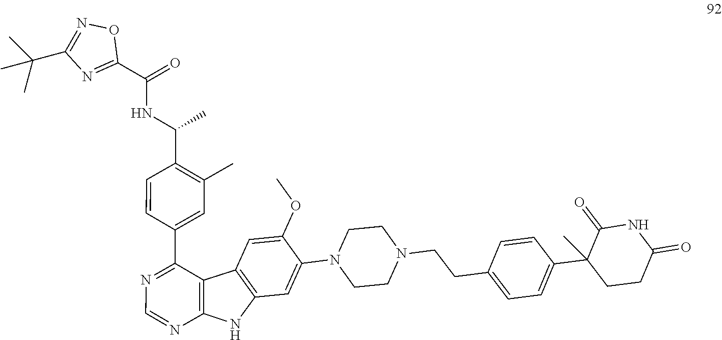 [SMILES: COc1cc2c(cc1N1CCN(CCc3ccc(C4(C)CCC(=O)NC4=O)cc3)CC1)[nH]c1ncnc(-c3ccc([C@@H](C)NC(=O)c4nc(C(C)(C)C)no4)c(C)c3)c12]

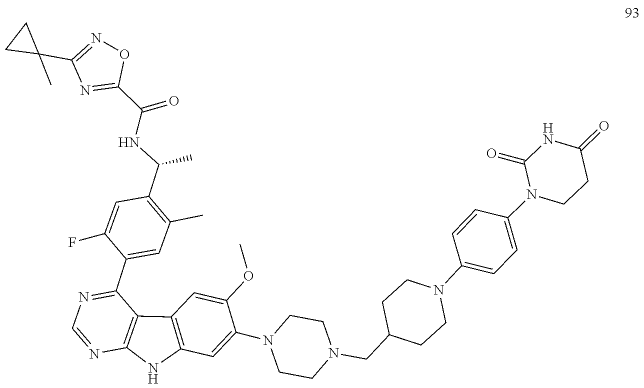 [SMILES: COc1cc2c(cc1N1CCN(CC3CCN(c4ccc(N5CCC(=O)NC5=O)cc4)CC3)CC1)[nH]c1ncnc(-c3cc(C)c([C@@H](C)NC(=O)c4nc(C5(C)CC5)no4)cc3F)c12]

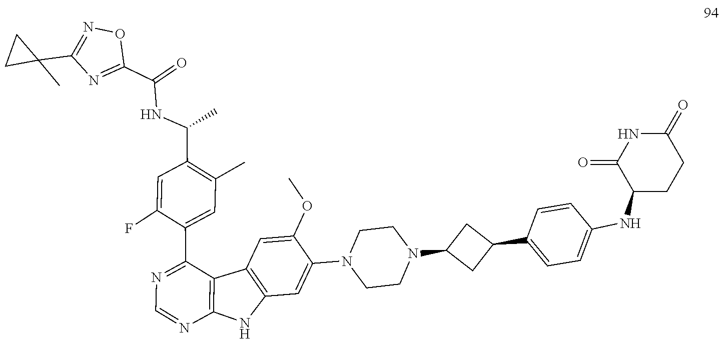 [SMILES: COc1cc2c(cc1N1CCN([C@H]3C[C@@H](c4ccc(N[C@@H]5CCC(=O)NC5=O)cc4)C3)CC1)[nH]c1ncnc(-c3cc(C)c([C@@H](C)NC(=O)c4nc(C5(C)CC5)no4)cc3F)c12]

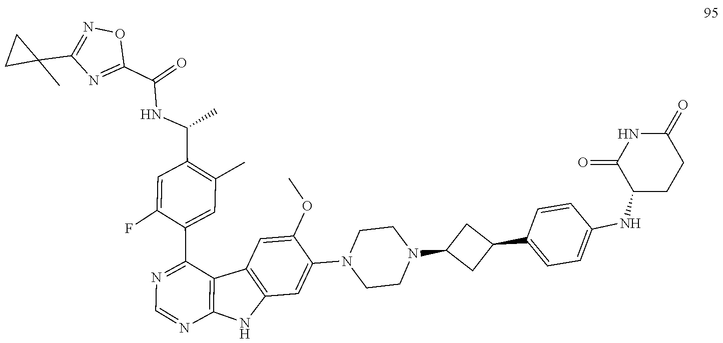 [SMILES: COc1cc2c(cc1N1CCN([C@H]3C[C@@H](c4ccc(N[C@H]5CCC(=O)NC5=O)cc4)C3)CC1)[nH]c1ncnc(-c3cc(C)c([C@@H](C)NC(=O)c4nc(C5(C)CC5)no4)cc3F)c12]

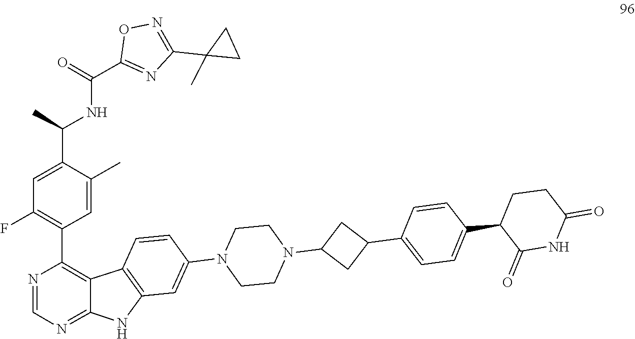 [SMILES: Cc1cc(-c2ncnc3[nH]c4cc(N5CCN(C6CC(c7ccc([C@H]8CCC(=O)NC8=O)cc7)C6)CC5)ccc4c23)c(F)cc1[C@@H](C)NC(=O)c1nc(C2(C)CC2)no1]

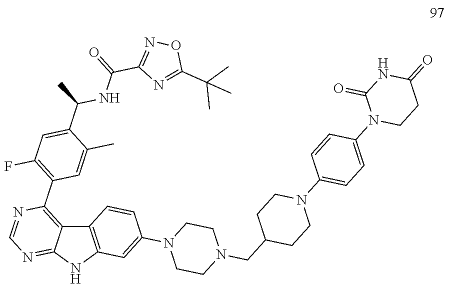 [SMILES: Cc1cc(-c2ncnc3[nH]c4cc(N5CCN(CC6CCN(c7ccc(N8CCC(=O)NC8=O)cc7)CC6)CC5)ccc4c23)c(F)cc1[C@@H](C)NC(=O)c1noc(C(C)(C)C)n1]

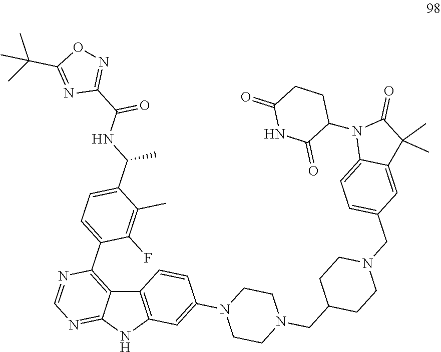 [SMILES: Cc1c([C@@H](C)NC(=O)c2noc(C(C)(C)C)n2)ccc(-c2ncnc3[nH]c4cc(N5CCN(CC6CCN(Cc7ccc8c(c7)C(C)(C)C(=O)N8C7CCC(=O)NC7=O)CC6)CC5)ccc4c23)c1F]

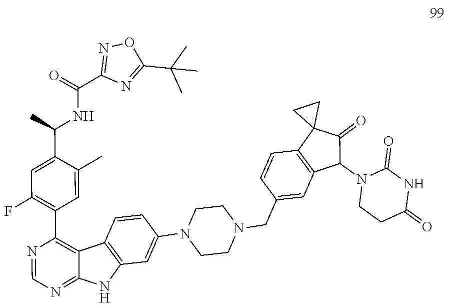 [SMILES: Cc1cc(-c2ncnc3[nH]c4cc(N5CCN(Cc6ccc7c(c6)C(N6CCC(=O)NC6=O)C(=O)C76CC6)CC5)ccc4c23)c(F)cc1[C@@H](C)NC(=O)c1noc(C(C)(C)C)n1]

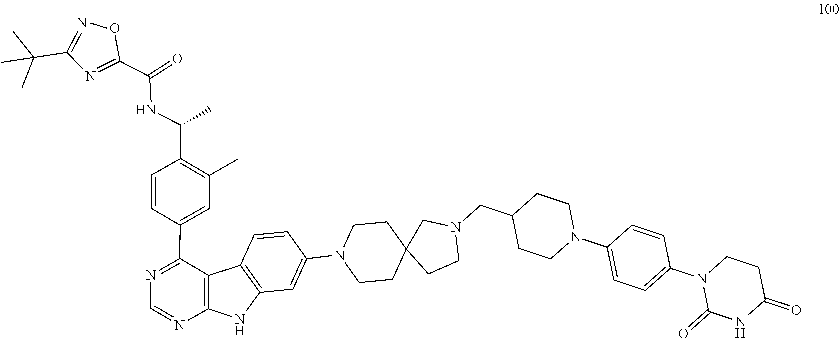 [SMILES: Cc1cc(-c2ncnc3[nH]c4cc(N5CCC6(CCN(CC7CCN(c8ccc(N9CCC(=O)NC9=O)cc8)CC7)C6)CC5)ccc4c23)ccc1[C@@H](C)NC(=O)c1nc(C(C)(C)C)no1]

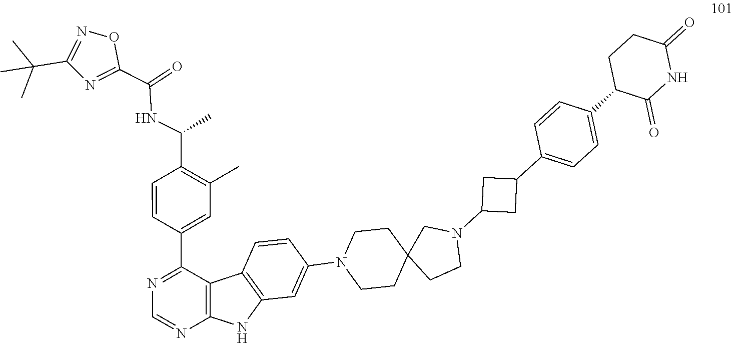 [SMILES: Cc1cc(-c2ncnc3[nH]c4cc(N5CCC6(CC5)CCN(C5CC(c7ccc([C@@H]8CCC(=O)NC8=O)cc7)C5)C6)ccc4c23)ccc1[C@@H](C)NC(=O)c1nc(C(C)(C)C)no1]

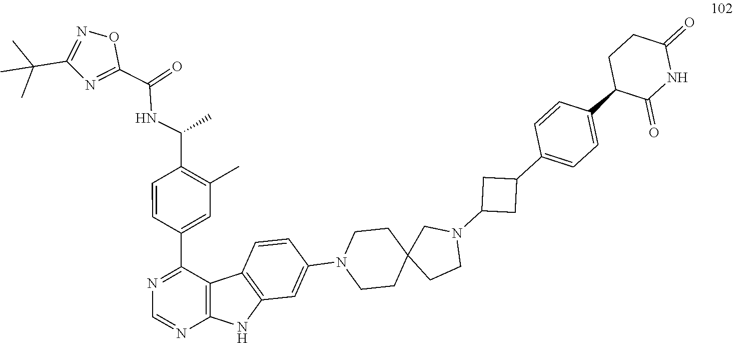 [SMILES: Cc1cc(-c2ncnc3[nH]c4cc(N5CCC6(CC5)CCN(C5CC(c7ccc([C@H]8CCC(=O)NC8=O)cc7)C5)C6)ccc4c23)ccc1[C@@H](C)NC(=O)c1nc(C(C)(C)C)no1]

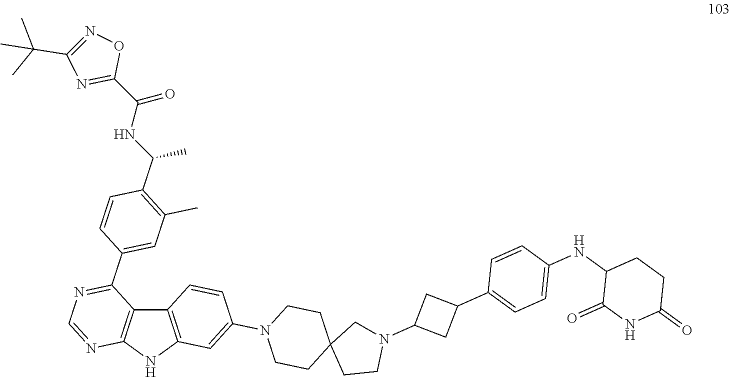 [SMILES: Cc1cc(-c2ncnc3[nH]c4cc(N5CCC6(CC5)CCN(C5CC(c7ccc(NC8CCC(=O)NC8=O)cc7)C5)C6)ccc4c23)ccc1[C@@H](C)NC(=O)c1nc(C(C)(C)C)no1]

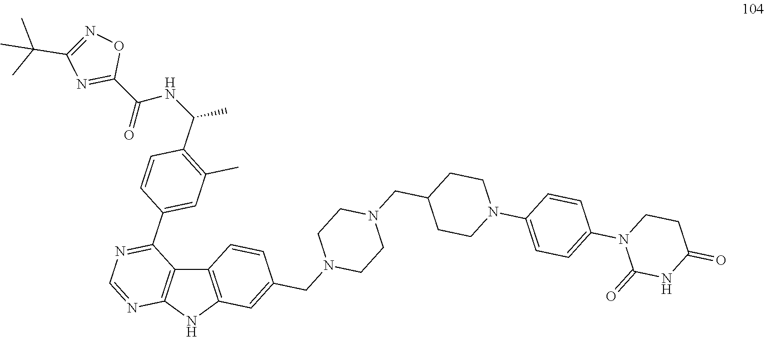 [SMILES: Cc1cc(-c2ncnc3[nH]c4cc(CN5CCN(CC6CCN(c7ccc(N8CCC(=O)NC8=O)cc7)CC6)CC5)ccc4c23)ccc1[C@@H](C)NC(=O)c1nc(C(C)(C)C)no1]

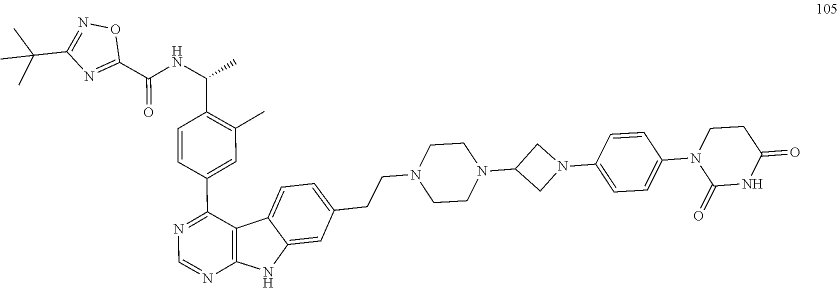 [SMILES: Cc1cc(-c2ncnc3[nH]c4cc(CCN5CCN(C6CN(c7ccc(N8CCC(=O)NC8=O)cc7)C6)CC5)ccc4c23)ccc1[C@@H](C)NC(=O)c1nc(C(C)(C)C)no1]

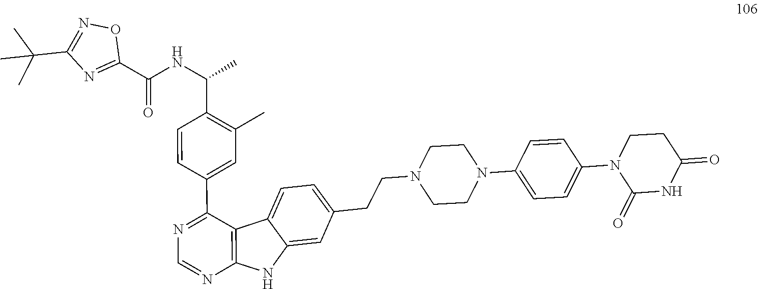 [SMILES: Cc1cc(-c2ncnc3[nH]c4cc(CCN5CCN(c6ccc(N7CCC(=O)NC7=O)cc6)CC5)ccc4c23)ccc1[C@@H](C)NC(=O)c1nc(C(C)(C)C)no1]

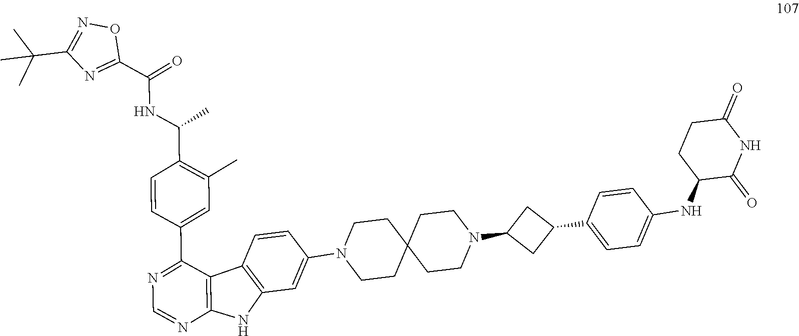 [SMILES: Cc1cc(-c2ncnc3[nH]c4cc(N5CCC6(CC5)CCN([C@H]5C[C@H](c7ccc(N[C@H]8CCC(=O)NC8=O)cc7)C5)CC6)ccc4c23)ccc1[C@@H](C)NC(=O)c1nc(C(C)(C)C)no1]

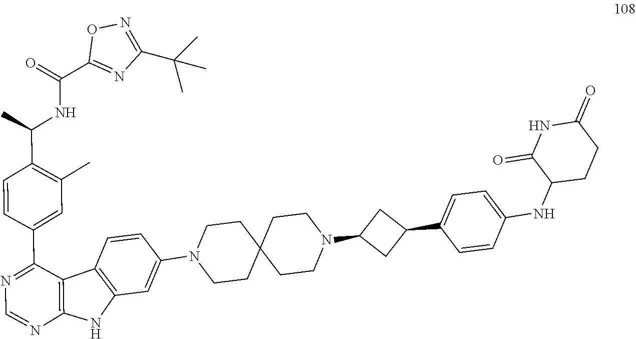 [SMILES: Cc1cc(-c2ncnc3[nH]c4cc(N5CCC6(CC5)CCN([C@H]5C[C@@H](c7ccc(NC8CCC(=O)NC8=O)cc7)C5)CC6)ccc4c23)ccc1[C@@H](C)NC(=O)c1nc(C(C)(C)C)no1]

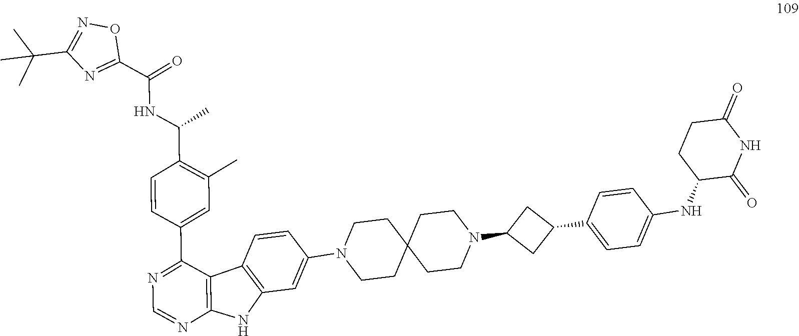 [SMILES: Cc1cc(-c2ncnc3[nH]c4cc(N5CCC6(CC5)CCN([C@H]5C[C@H](c7ccc(N[C@@H]8CCC(=O)NC8=O)cc7)C5)CC6)ccc4c23)ccc1[C@@H](C)NC(=O)c1nc(C(C)(C)C)no1]

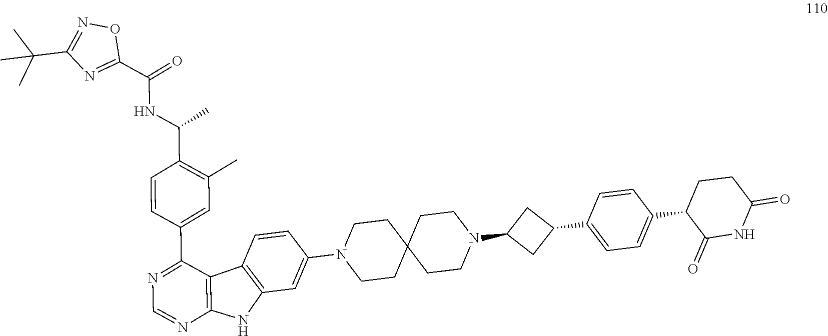 [SMILES: Cc1cc(-c2ncnc3[nH]c4cc(N5CCC6(CC5)CCN([C@H]5C[C@H](c7ccc([C@@H]8CCC(=O)NC8=O)cc7)C5)CC6)ccc4c23)ccc1[C@@H](C)NC(=O)c1nc(C(C)(C)C)no1]

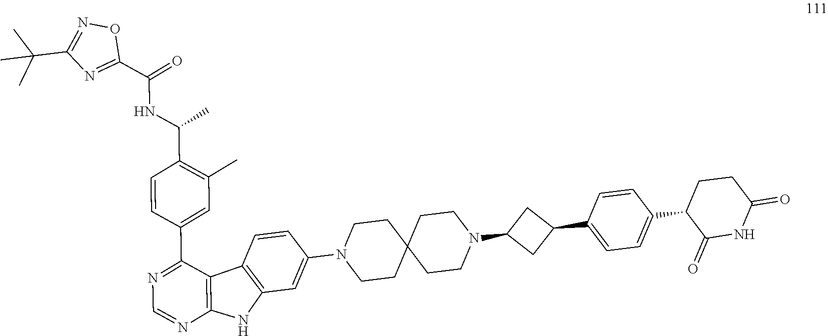 [SMILES: Cc1cc(-c2ncnc3[nH]c4cc(N5CCC6(CC5)CCN([C@H]5C[C@@H](c7ccc([C@@H]8CCC(=O)NC8=O)cc7)C5)CC6)ccc4c23)ccc1[C@@H](C)NC(=O)c1nc(C(C)(C)C)no1]

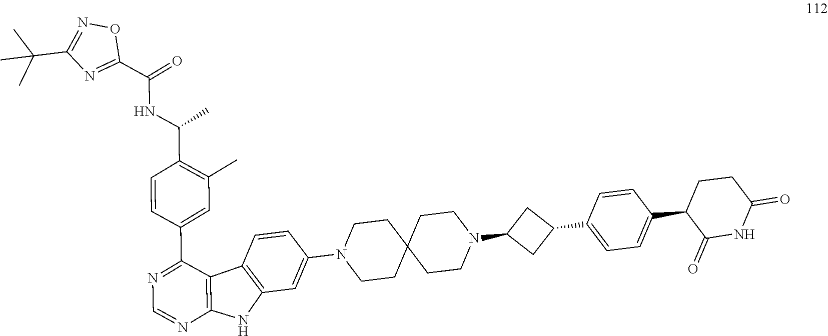 [SMILES: Cc1cc(-c2ncnc3[nH]c4cc(N5CCC6(CC5)CCN([C@H]5C[C@H](c7ccc([C@H]8CCC(=O)NC8=O)cc7)C5)CC6)ccc4c23)ccc1[C@@H](C)NC(=O)c1nc(C(C)(C)C)no1]

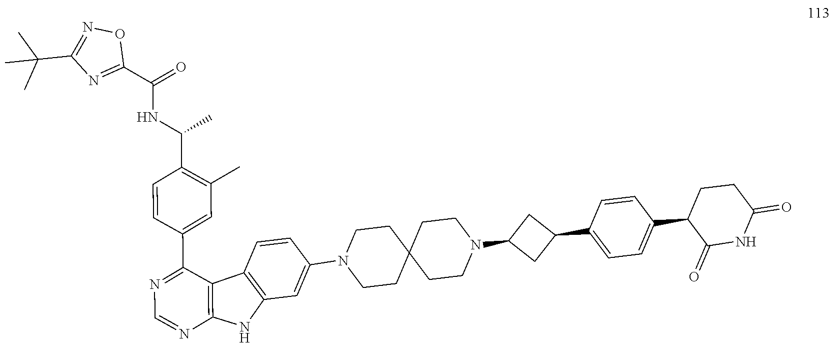 [SMILES: Cc1cc(-c2ncnc3[nH]c4cc(N5CCC6(CC5)CCN([C@H]5C[C@@H](c7ccc([C@H]8CCC(=O)NC8=O)cc7)C5)CC6)ccc4c23)ccc1[C@@H](C)NC(=O)c1nc(C(C)(C)C)no1]

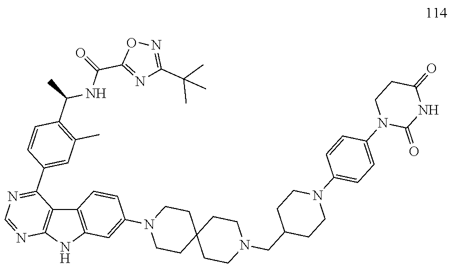 [SMILES: Cc1cc(-c2ncnc3[nH]c4cc(N5CCC6(CCN(CC7CCN(c8ccc(N9CCC(=O)NC9=O)cc8)CC7)CC6)CC5)ccc4c23)ccc1[C@@H](C)NC(=O)c1nc(C(C)(C)C)no1]

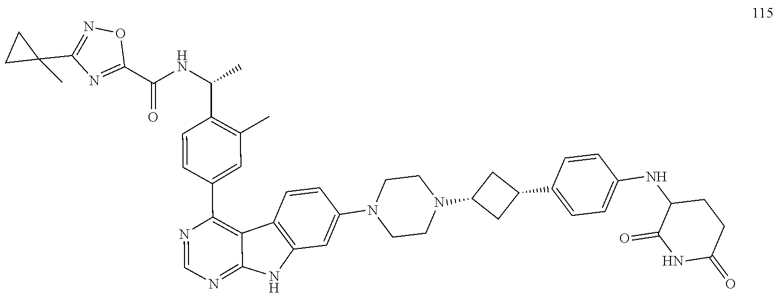 [SMILES: Cc1cc(-c2ncnc3[nH]c4cc(N5CCN([C@H]6C[C@@H](c7ccc(NC8CCC(=O)NC8=O)cc7)C6)CC5)ccc4c23)ccc1[C@@H](C)NC(=O)c1nc(C2(C)CC2)no1]

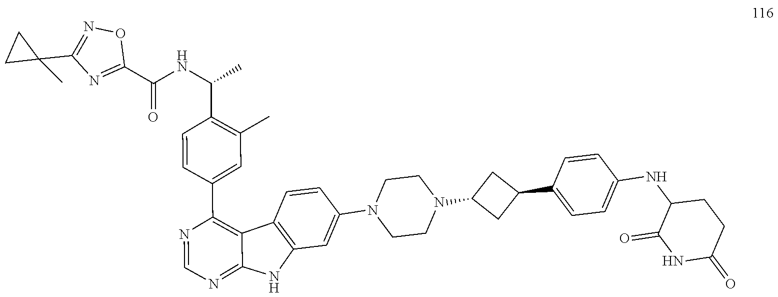 [SMILES: Cc1cc(-c2ncnc3[nH]c4cc(N5CCN([C@H]6C[C@H](c7ccc(NC8CCC(=O)NC8=O)cc7)C6)CC5)ccc4c23)ccc1[C@@H](C)NC(=O)c1nc(C2(C)CC2)no1]

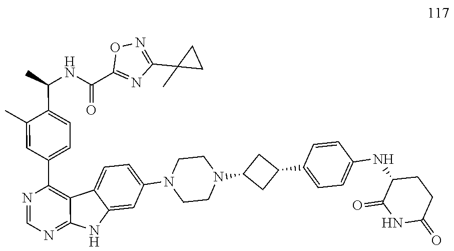 [SMILES: Cc1cc(-c2ncnc3[nH]c4cc(N5CCN([C@H]6C[C@@H](c7ccc(N[C@@H]8CCC(=O)NC8=O)cc7)C6)CC5)ccc4c23)ccc1[C@@H](C)NC(=O)c1nc(C2(C)CC2)no1]

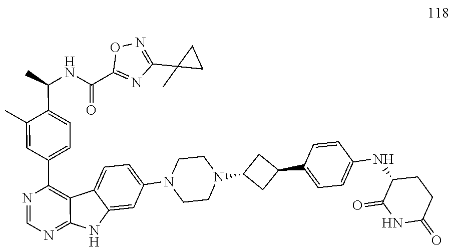 [SMILES: Cc1cc(-c2ncnc3[nH]c4cc(N5CCN([C@H]6C[C@H](c7ccc(N[C@@H]8CCC(=O)NC8=O)cc7)C6)CC5)ccc4c23)ccc1[C@@H](C)NC(=O)c1nc(C2(C)CC2)no1]

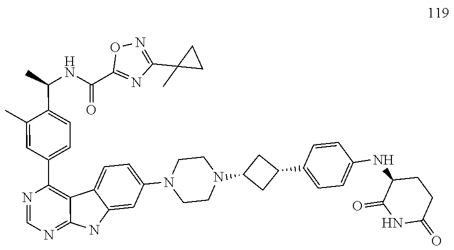

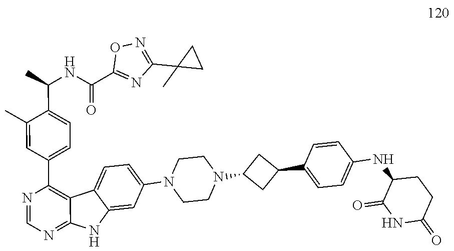 [SMILES: Cc1cc(-c2ncnc3[nH]c4cc(N5CCN([C@H]6C[C@H](c7ccc(N[C@H]8CCC(=O)NC8=O)cc7)C6)CC5)ccc4c23)ccc1[C@@H](C)NC(=O)c1nc(C2(C)CC2)no1]

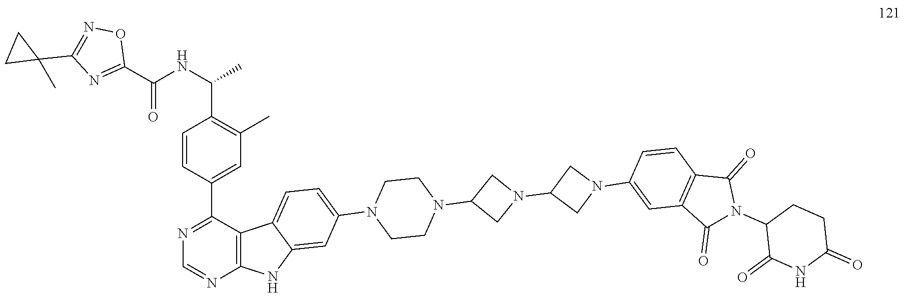 [SMILES: Cc1cc(-c2ncnc3[nH]c4cc(N5CCN(C6CN(C7CN(c8ccc9c(c8)C(=O)N(C8CCC(=O)NC8=O)C9=O)C7)C6)CC5)ccc4c23)ccc1[C@@H](C)NC(=O)c1nc(C2(C)CC2)no1]

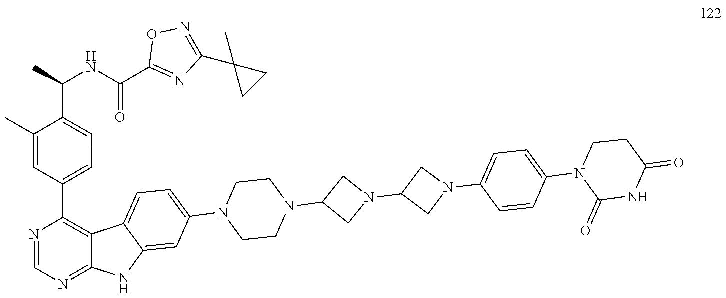 [SMILES: Cc1cc(-c2ncnc3[nH]c4cc(N5CCN(C6CN(C7CN(c8ccc(N9CCC(=O)NC9=O)cc8)C7)C6)CC5)ccc4c23)ccc1[C@@H](C)NC(=O)c1nc(C2(C)CC2)no1]

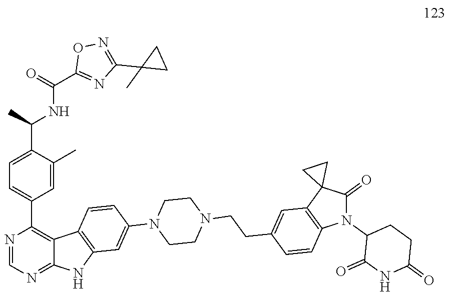 [SMILES: Cc1cc(-c2ncnc3[nH]c4cc(N5CCN(CCc6ccc7c(c6)C6(CC6)C(=O)N7C6CCC(=O)NC6=O)CC5)ccc4c23)ccc1[C@@H](C)NC(=O)c1nc(C2(C)CC2)no1]

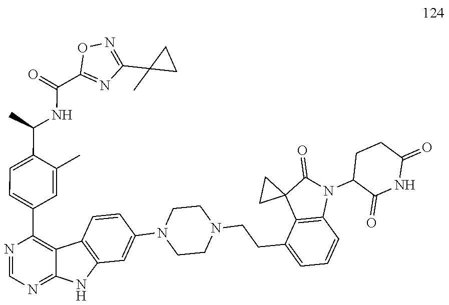 [SMILES: Cc1cc(-c2ncnc3[nH]c4cc(N5CCN(CCc6cccc7c6C6(CC6)C(=O)N7C6CCC(=O)NC6=O)CC5)ccc4c23)ccc1[C@@H](C)NC(=O)c1nc(C2(C)CC2)no1]

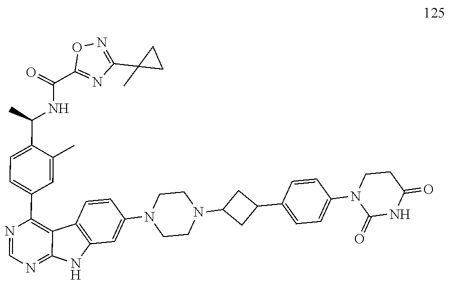 [SMILES: Cc1cc(-c2ncnc3[nH]c4cc(N5CCN(C6CC(c7ccc(N8CCC(=O)NC8=O)cc7)C6)CC5)ccc4c23)ccc1[C@@H](C)NC(=O)c1nc(C2(C)CC2)no1]

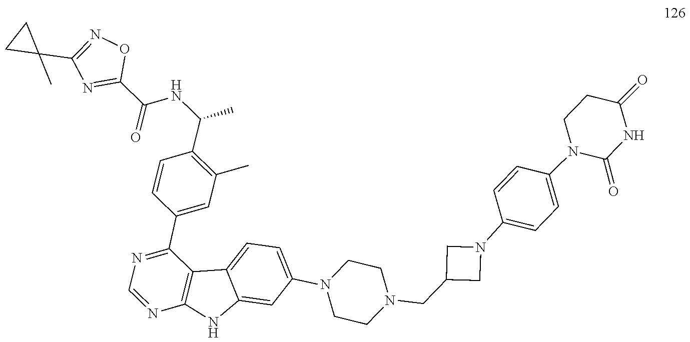 [SMILES: Cc1cc(-c2ncnc3[nH]c4cc(N5CCN(CC6CN(c7ccc(N8CCC(=O)NC8=O)cc7)C6)CC5)ccc4c23)ccc1[C@@H](C)NC(=O)c1nc(C2(C)CC2)no1]

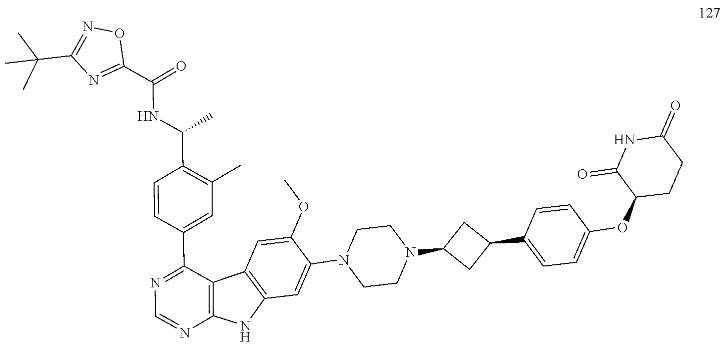 [SMILES: COc1cc2c(cc1N1CCN([C@H]3C[C@@H](c4ccc(O[C@@H]5CCC(=O)NC5=O)cc4)C3)CC1)[nH]c1ncnc(-c3ccc([C@@H](C)NC(=O)c4nc(C(C)(C)C)no4)c(C)c3)c12]

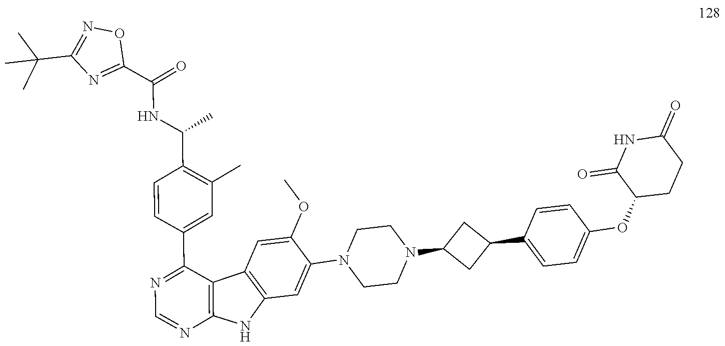 [SMILES: COc1cc2c(cc1N1CCN([C@H]3C[C@@H](c4ccc(O[C@H]5CCC(=O)NC5=O)cc4)C3)CC1)[nH]c1ncnc(-c3ccc([C@@H](C)NC(=O)c4nc(C(C)(C)C)no4)c(C)c3)c12]

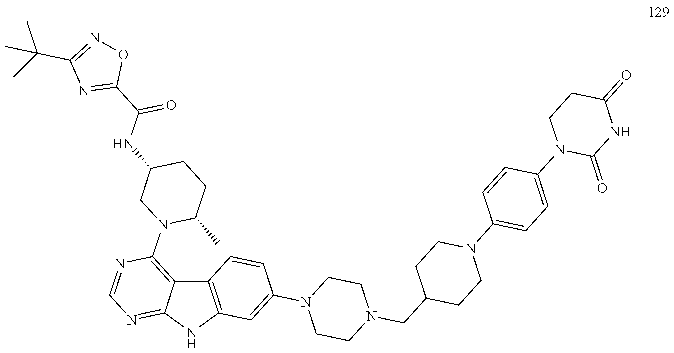 [SMILES: C[C@H]1CC[C@@H](NC(=O)c2nc(C(C)(C)C)no2)CN1c1ncnc2[nH]c3cc(N4CCN(CC5CCN(c6ccc(N7CCC(=O)NC7=O)cc6)CC5)CC4)ccc3c12]

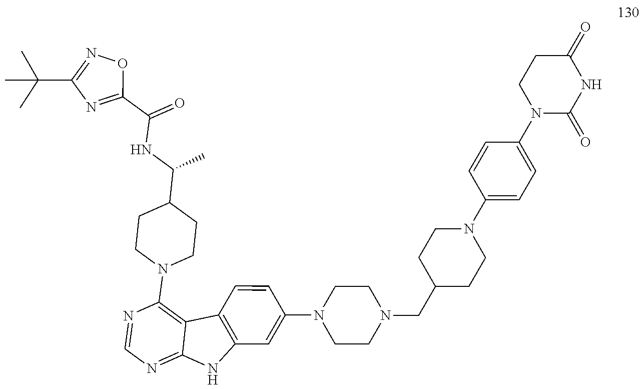 [SMILES: C[C@@H](NC(=O)c1nc(C(C)(C)C)no1)C1CCN(c2ncnc3[nH]c4cc(N5CCN(CC6CCN(c7ccc(N8CCC(=O)NC8=O)cc7)CC6)CC5)ccc4c23)CC1]

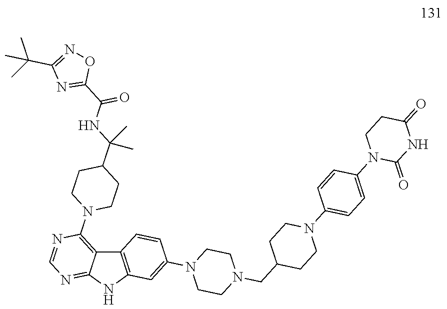 [SMILES: CC(C)(C)c1noc(C(=O)NC(C)(C)C2CCN(c3ncnc4[nH]c5cc(N6CCN(CC7CCN(c8ccc(N9CCC(=O)NC9=O)cc8)CC7)CC6)ccc5c34)CC2)n1]

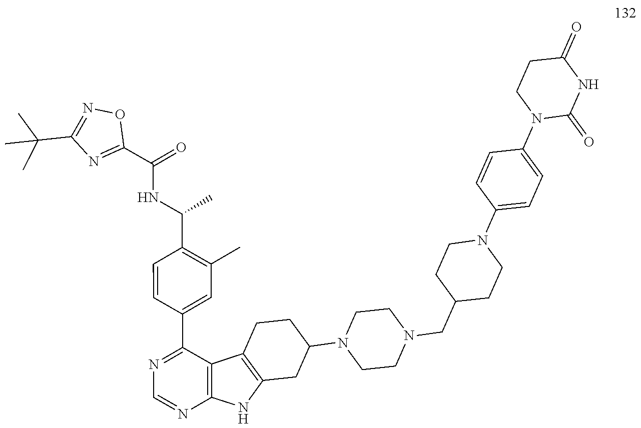 [SMILES: Cc1cc(-c2ncnc3[nH]c4c(c23)CCC(N2CCN(CC3CCN(c5ccc(N6CCC(=O)NC6=O)cc5)CC3)CC2)C4)ccc1[C@@H](C)NC(=O)c1nc(C(C)(C)C)no1]

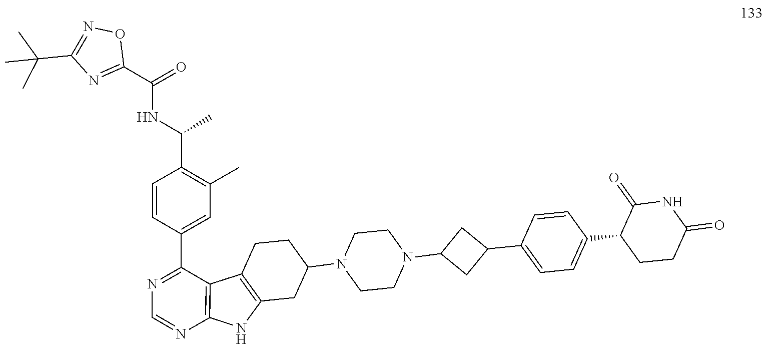 [SMILES: Cc1cc(-c2ncnc3[nH]c4c(c23)CCC(N2CCN(C3CC(c5ccc([C@H]6CCC(=O)NC6=O)cc5)C3)CC2)C4)ccc1[C@@H](C)NC(=O)c1nc(C(C)(C)C)no1]

38. A pharmaceutical composition comprising the compound according to claim 1, or a pharmaceutically acceptable salt thereof, and at least one pharmaceutically acceptable carrier or excipient.

39. A method of inhibiting BTK activity, which comprises administering to an individual the compound according to claim 1, or a pharmaceutically acceptable salt thereof, including the compound of formula (I) or the specific compounds exemplified herein.

40. A method of treating a disease or disorder in a patient comprising administering to the patient a therapeutically effective amount of the compound of claim 1, or a pharmaceutically acceptable salt thereof as an BTK kinase inhibitor, wherein the disease or disorder is associated with inhibition of BTK, preferably, the disease or disorder is cancer.
